(19) United States
(12) Patent Application Publication
(10) Pub. No.: US 2024/0018125 A1
(43) Pub. Date: Jan. 18, 2024

(54) CYCLIC ISOTHIOUREA DERIVATIVES AS CXCR4 MODULATORS

(71) Applicants: ERMIUM THERAPEUTICS, Paris (FR); UNIVERSITÉ DE PARIS, Paris (FR); CENTRE NATIONAL DE LA RECHERCHE SCIENTIFIQUE, Paris Cedex 16 (FR)

(72) Inventors: Jean-Philippe HERBEUVAL, Paris (FR); Anaïs BARRÉ, Illkirch Graffenstaden (FR); Julie CASSOU, Eschau (FR); Stanislas MAYER, Eschau (FR); Patrick BAZZINI, Strasbourg (FR)

(21) Appl. No.: 18/028,637

(22) PCT Filed: Sep. 28, 2021
(86) PCT No.: PCT/EP2021/076715

§ 371 Date: Mar. 27, 2023

(30) Foreign Application Priority Data
Sep. 28, 2020 (EP) 20306111.4

(51) Int. Cl. C07D 403/12; C07D 513/04; C07D 401/12; C07D 487/04; A61P 37/02

(52) U.S. Cl. CPC C07D 403/12; C07D 513/04; A61P 37/02; C07D 487/04; C07D 401/12

(57) ABSTRACT

The present invention provides novel compounds of formula (I) and pharmaceutical compositions containing these compounds. The compounds of formula (I) can act as CXCR4 modulators that specifically target the CXCR4 minor pocket, and they have further been found to inhibit the production of inflammatory cytokines in immune cells, which renders these compounds highly advantageous for use in therapy, particularly in the treatment or prevention of an inflammatory disorder, an autoimmune disorder, an autoinflammatory disorder, or an interferonopathy, such as, e.g., systemic lupus erythematosus, dermatomyositis or rheumatoid arthritis.

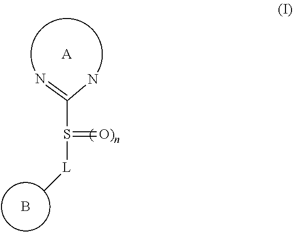

The present invention provides novel compounds of formula (I) and pharmaceutical compositions containing these compounds. The compounds of formula (I) can act as CXCR4 modulators that specifically target the CXCR4 minor pocket, and they have further been found to inhibit the production of inflammatory cytokines in immune cells, which renders these compounds highly advantageous for use in therapy, particularly in the treatment or prevention of an inflammatory disorder, an autoimmune disorder, an autoinflammatory disorder, or an interferonopathy, such as, e.g., systemic lupus erythematosus, dermatomyositis or rheumatoid arthritis.

Disorders of the immune system are at the basis of numerous diseases that can be divided into two categories: autoinflammatory diseases that affect the innate immune system and autoimmune diseases that involve the adaptive immune system. In both cases, the immune system attacks the normal constituents of the organism considering them as foreign. It becomes pathogenic and induces lesions on a specific organ (e.g., type 1 diabetes in the pancreas or multiple sclerosis in the brain) or systemically (e.g., rheumatoid arthritis or systemic lupus erythematosus, SLE). These diseases evolve chronically, with phases of relapse and remission. At the origin of these dysfunctions, the failure of self-tolerance mechanisms is induced by multiple genetic, hormonal and environmental factors and is still largely misunderstood.

Some autoimmune diseases are rare, affecting less than one in five thousand individuals. But taken as a whole, they are common, affect mainly women and their overall prevalence is about 5 to 10%. As an example, rheumatoid arthritis is one of the most frequent with an estimated prevalence in France at 1000 to 4000 per 100 000 women (4 times less for men).

Cytokines are small proteins involved in cell signaling that orchestrate the immune response. Targeting them has therefore become a real therapeutic option for autoimmune and autoinflammatory diseases but also for chronic viral infections and inflammatory diseases such as sepsis.

Interferonopathies

Type I interferons (IFN-I) are key immune response mediators and in humans are composed of 13 IFN-alpha (IFN-α) subtypes as well as IFN-μ, IFN-ε, IFN-κ and IFN-ω. Type I IFNs signal through a common receptor (IFNAR) ubiquitously expressed and formed by two transmembrane proteins, IFNAR1 and IFNAR2. IFNAR engagement results in activation of the cytoplasmic kinases JAK1 and TYK2 leading to the formation of the transcription factor complex ISGF3. This complex translocates to the nucleus to promote transcription of IFN-stimulated genes (ISG). Type-I IFNs have antiproliferative and immunomodulatory effects and are essential to control viral infection and spread. However, sustained overproduction of IFN-I can be deleterious for the host. The negative impact of IFN-I is well illustrated by a class of disorders collectively termed type 1 interferonopathies (Gitiaux C et al., Arthritis Rheumatol, 2018, 70, 134-145; Melki I et al., J Allergy Clin Immunol, 2017, 140, 543-552 e545; Rice G I et al., J Clin Immunol, 2017, 37, 123-132; Rodero M P et al., J Exp Med, 2017, 214, 1547-1555; Rodero M P et al., Nat Commun, 2017, 8, 2176), which include rare monogenic diseases and complex autoinflammatory/autoimmune diseases such as systemic lupus erythematous (SLE).

Autoinflammation and Autoimmunity Triggered by Type I Interferon

The type I interferonopathies comprise a growing number of genetically determined disorders that are primarily caused by perturbations of the innate immune system. The term type I interferonopathy was coined in recognition of an abnormal upregulation of type I IFN as a unifying phenotype of this novel group of diseases (Crow Y J, Curr Opin Immunol, 2015, 32, 7-12). Despite a remarkable phenotypic heterogeneity, type I interferonopathies are commonly characterized by systemic autoinflammation and varying degrees of autoimmunity or immunodeficiency. Based on the currently identified molecular defects, a pathogenic type I IFN response can result from (a) abnormal accumulation of or abnormal chemical modification of endogenous nucleic acids, (b) enhanced sensitivity or ligand-independent activation of nucleic acid sensors or of downstream components of type I IFN signaling pathways, (c) impaired negative regulation of nucleic acid-induced type I IFN signaling, or (d) defects in pathways that modulate type I IFN responses independent of nucleic acid sensing (Lee-Kirsch M A, Annu Rev Med, 2017, 68, 297-315).

Type I interferonopathies include, for example, Aicardi-Goutières syndrome (AGS), retinal vasculopathy with cerebral leukodystrophy (RVCL), familial chilblain lupus (CHBL), systemic lupus erythematosus (SLE), STING-associated vasculopathy with onset in infancy (SAVI), Singleton-Merten syndrome (SGMRT), spondyloenchondrodysplasia (SPENCD), ISG15 deficiency, proteasome-associated autoinflammatory syndrome, and deficiency of adenosine deaminase 2.

The development of therapies aiming to inhibit type I IFN production in autoimmune diseases has been stimulated by the observation that type I IFNAR knock-out murine lupus models have a reduced disease activity. Although upregulation of IFNs in SLE has been detected for a long time, however, the anti-IFN therapy developed very slowly (Felten R et al., Autoimmunity reviews, 2018, 17, 781-790). It is not only due to no effective approach to block IFN but also the high risk to induce infection or tumor induced by anti-IFN therapy (Crow M K, Rheumatic diseases clinics of North America, 2010, 36, 173-186). There are possible methods to downregulate the IFN pathway in SLE, but a more personalized approach to modulate the type I IFN system in order to reduce the risk for increased frequency and severity of infectious diseases would be a major therapeutic leap forward for this vulnerable group of patients. At this moment, a number of clinical trials are in progress. Given promising results, the efficacy of sifalimumab, an IFN-α monoclonal antibody, is now being investigated in phase II clinical trials. Based on preliminary results, experimental group has an obvious improvement compared with placebo group and a single injection of an anti-IFN-α antibody could give a sustained neutralization of the IFN signature. In this study, researchers found that sifalimumab suppresses IFN-α level not only in whole blood but also in skin tissue of SLE. So far, no increase in serious viral infections has been reported among anti-IFN-α-treated patients, which could be due to the fact that, besides IFN-α, several other type I IFNs exist with strong antiviral activity.

Autoinflammatory Diseases

Autoinflammatory diseases are conditions where inflammatory cytokines are involved in the pathogenesis. They are characterized by immune activation, infiltration and abnormal cytokine production. They include conditions such as: rheumatologic inflammatory diseases, skin inflammatory diseases, lung inflammatory diseases, muscle inflammatory diseases, bowel inflammatory diseases, brain inflammatory diseases and autoimmune diseases.

Among this large panel of diseases, rheumatoid arthritis (RA) is along-term autoimmune disorder that primarily affects joints (Smolen J S et al., The Lancet, 2016, 388, 2023-2038). It typically results in warm, swollen, and painful joints. Pain and stiffness often worsen following rest. Most commonly, the wrist and hands are involved, with the same joints typically involved on both sides of the body (https://www.niams.nih.gov/health-topics/rheumatoid-arthritis). The disease may also affect other parts of the body (Smolen J S et al., The Lancet, 2016, 388, 2023-2038). This may result in a low red blood cell count, and inflammation around the heart. Fever and low energy may also be present. Often, symptoms come on gradually over weeks to months. While the cause of rheumatoid arthritis is not clear, it is believed to involve a combination of genetic and environmental factors (https://www.niams.nih.gov/health-topics/rheumatoid-arthritis). The underlying mechanism involves the body's immune system attacking the joints (Smolen J S et al., The Lancet, 2016, 388, 2023-2038). This results in inflammation and thickening of the joint capsule (Smolen J S et al., The Lancet, 2016, 388, 2023-2038). It also affects the underlying bone and cartilage (Smolen J S et al., The Lancet, 2016, 388, 2023-2038). The diagnosis is made mostly on the basis of a person's signs and symptoms. X-rays and laboratory testing may support a diagnosis or exclude other diseases with similar symptoms (Smolen J S et al., The Lancet, 2016, 388, 2023-2038). Other diseases that may present similarly include systemic lupus erythematosus, psoriatic arthritis, and fibromyalgia among others.

The goals of treatment are to reduce pain, decrease inflammation, and improve a person's overall functioning. This may be helped by balancing rest and exercise, the use of splints and braces, or the use of assistive devices. Pain medications, steroids, and NSAIDs are frequently used to help with symptoms. Disease-modifying antirheumatic drugs (DMARDs), such as hydroxychloroquine and methotrexate, may be used to try to slow the progression of disease. Biological DMARDs may be used when disease does not respond to other treatments. However, they may have a greater rate of adverse effects. Surgery to repair, replace, or fuse joints may help in certain situations.

Inflammatory Diseases

While autoimmune and autoinflammatory diseases evolve chronically, some conditions can lead to an acute immune disorder. Indeed, a sudden excessive and uncontrolled release of pro-inflammatory cytokines, also called cytokine storm, has been observed in graft-versus-host disease, multiple sclerosis, pancreatitis, multiple organ dysfunction syndrome, viral diseases, bacterial infections, hemophagocytic lymphohistiocytosis, and sepsis (Gerlach H, F1000Res, 2016, 5, 2909; Tisoncik J R et al., Microbiol Mol Biol Rev, 2012, 76(1), 16-32). In these conditions, a dysregulated immune response and subsequent hyperinflammation may lead to multiple organ failure that can be fatal.

Sepsis is a systemic inflammatory response to infection with highly variable clinical manifestations (Angus D C et al., N Engl J Med, 2013, 369(9), 840-851). Acute organ dysfunction commonly affects the respiratory and cardiovascular system with acute respiratory distress syndrome (ARDS) and hypotension or elevated serum lactate level. The brain and kidneys are also often affected leading to obtundation, delirium, polyneuropathy, myopathy or acute kidney injuries (Angus D C et al., N Engl J Med, 2013, 369(9), 840-851).

Treatment of sepsis consists in 2 phases. The initial management within the first 6 hours after the patient's presentation consists in providing cardiorespiratory resuscitation (fluids, vasopressors, oxygen therapy and mechanical ventilation) and controlling the infection (antibiotics). After these first 6 hours, attention focuses on supporting organ functions and avoiding complications. In this second part, immunomodulatory therapy such as hydrocortisone can be administered (Angus D C et al., N Engl J Med, 2013, 369(9), 840-851).

Despite substantial advances in modern intensive care, mortality in sepsis patients is still close to 20 to 30%. Patients who survive sepsis remain at risk for death in the following months and years and often have impaired physical or neurocognitive functioning, mood disorders and low quality of life (Angus D C et al., N Engl J Med, 2013, 369(9), 840-851). Therefore, New Therapeutic Strategies are Urgently Needed.

CXCR4 as therapeutic target

CXCR4 is a well-known chemokine receptor described for its role in cell migration (chemotaxis). CXCR4 expression has been reported in most hematopoietic cell types, including neutrophils, monocytes, B and T lymphocytes, CD34+ progenitor cells, immature and mature dendritic cells, and platelets. It is also highly expressed in vascular endothelial cells, neurons, microglia, astrocytes and several types of cancer cells. Upon injury, the blockade of the interaction between CXCR4 and its ligand CXCL12 or SDF1-α enhances progenitor cell mobilization from the bone marrow to the periphery. Similarly, CXCR4 influences trafficking of other immune cells, but also CXCR4-positive cancer cells. Furthermore, CXCR4 and CCR5 are coreceptors involved in Human Immunodeficiency Virus (HIV) entry into CD4+ T cells in humans. Based on these functions, CXCR4 has been widely studied by the pharmaceutical industries. For example, the CXCR4 antagonist AMD3100 or plerixafor is clinically approved for the mobilization of hematopoietic progenitor cells for autologous transplantations in patients with lymphoma and multiple myeloma. Antagonists of CXCR4 are also actively developed to prevent the migration of CXCR4-expressing cancer cells either to prevent metastasis of solid tumors or the homing of leukemic cells in the bone marrow which is associated with drug resistance.

Numerous CXCR4 ligands have been described including pyridines, quinolones, peptides or polyazamacrocycles with a large range of affinities (Debnath B et al., Theranostics, 2013, 3, 47-75). CXCR4 is overexpressed by activated immune cells in autoimmune and autoinflammatory disease patients (Wang A et al., Arthritis and Rheumatism, 2010, 62, 3436-3446). It has further been demonstrated that engagement of CXCR4 by natural amine and the synthetic mimic of histamine (clobenpropit) strongly inhibits viral-induced production of inflammatory cytokines on primary human peripheral Dendritic Cells (pDC) (Smith N et al., Nat Commun, 2017, 8, 14253; WO 2017/216368). In order to identify synthetic compounds with similar properties, known CXCR4 ligands with similar structures were searched. Excitingly, the first co-crystallized structure of CXCR4 was achieved with a small compound called IT1t showing a strong structural homology with clobenpropit (Wu B et al., Science, 2010, 330, 1066-1071). IT1t binds to an allosteric deep pocket that appeared to be distinct from the FDA approved CXCR4 ligand AMD3100 (plerixafor) binding site (Rosenkilde M M et al., J Biol Chem, 2007, 282, 27354-27365; Rosenkilde M M et al., J Biol Chem, 2004, 279, 3033-3041). These pockets were called major for the AMD3100 binding site and minor for IT1t (Wu B et al., Science, 2010, 330, 1066-1071) opening the possibility for distinct biological activity. A combination of structure- and ligand-based virtual screening of the “IT1t pocket” (Mishra R K et al., Scientific reports, 2016, 6, 30155) identified a set of small molecules with agonist or antagonist properties towards CXCL12 demonstrating for the first time the functionality of this pocket. The structural similarity between IT1t and clobenpropit (CB) and molecular modeling prediction support the idea of a common binding site. It has been demonstrated that, as observed with CB, IT1t also controls inflammation in vitro (production of interferons by dendritic cells, NK cells and monocytes) as well as in vivo in models of rheumatoid arthritis and systemic lupus erythematosus (Smith N et al., Sci Adv, 2019, 5, eaav9019; WO 2017/216368). This validates the CXCR4 minor pocket (IT1t binding pocket) as a tool to prevent the production of inflammatory cytokines in contrast to the CXCR4 major pocket of AMD3100 involved in cell migration (Rosenkilde M M et al., J Biol Chem, 2007, 282, 27354-27365; Wu B et al., Science, 2010, 330, 1066-1071). This work therefore clearly demonstrates that CXCR4 minor pocket-targeting molecules constitute a promising therapeutic strategy for inflammatory, autoimmune and autoinflammatory diseases as well as type I interferonopathies.

While various CXCR4 ligands have been reported in the literature (Debnath B et al., Theranostics, 2013, 3, 47-75; Thoma G et al., J Med Chem, 2008, 51(24), 7915-20; Wu B et al., Science, 2010, 330, 1066-1071; Rosenkilde M M et al., J Biol Chem, 2004, 279, 3033-3041; Rosenkilde M M et al., J Biol Chem, 2007, 282, 27354-27365; Mishra R K et al., Scientific reports, 2016, 6, 30155; Mona C E et al., Org Biomol Chem, 2016, 14(43), 10298-10311; Bai R et al., Eur J Med Chem, 2017, 126, 464-475; Mosley C A et al., Expert Opin Ther Pat, 2009, 19(1), 23-38; Smith N et al., Nat Commun, 2017, 8, 14253; Smith N et al., Sci Adv, 2019, 5, eaav9019; WO 2017/216368; EP-A-1 431 290), none of them has been shown to inhibit the production of inflammatory cytokines. Furthermore, long term inhibition of the CXCR4-CXCL12 signaling pathway can be highly toxic in vivo. Indeed, experiments with genetically modified animals have indicated that this pathway is essential for B lymphocyte development, maintenance of the hematopoietic stem cell pool in the bone marrow stromal cell niche, cardiac vascular formation, vascularization of the gastrointestinal tract, branching morphology in the pancreas, and cerebellar formation (Tsuchiya A et al., Dig Dis Sci, 2012, 57(11), 2892-2900). Chronic inhibition of the CXCR4-CXCL12 pathway has therefore a high risk of cardio-toxicity, muscle regeneration, neuro-protection or embryonic development disorders as well as increased risk of liver damages (Tsuchiya A et al., Dig Dis Sci, 2012, 57(11), 2892-2900; Li M et al, Trends Neurosci, 2012, 35(10), 619-628; Odemis V et al., Mol Cell Neurosci, 2005, 30(4), 494-505; Cash-Padgett T et al., Neurosci Res, 2016, 105, 75-79). This explains why, although current CXCR4 antagonists can be used via an acute or a chronic low dose administration, long-term high dose administrations have been avoided so far. It could also be extremely detrimental to use a treatment inducing the migration of immune cells, such as monocytes, from the bone marrow to the blood in a context of inflammatory, autoimmune or autoinflammatory disorder where these cells are responsible for the pathology. There is therefore still an unmet need for novel and improved CXCR4 modulators, particularly CXCR4 minor pocket-targeting molecules that block the pathogenic production of inflammatory cytokines while having minimal impact on the CXCR4-CXCL12 signaling, for the therapeutic intervention in inflammatory disorders, autoimmune disorders, autoinflammatory disorders, and interferonopathies.

In the context of the present invention, it has surprisingly been found that the compounds of formula (I) as provided herein are particularly potent inhibitors of the production of interferons and inflammatory cytokines by specifically targeting the CXCR4 minor pocket (IT1t binding pocket) while showing minimal to undetectable impact on the CXCR4-CXCL12 signaling pathway, which renders these compounds highly advantageous for use in therapy, particularly in the treatment or prevention of an inflammatory disorder, an autoimmune disorder, an autoinflammatory disorder, or an interferonopathy, such as, e.g., systemic lupus erythematosus, dermatomyositis or rheumatoid arthritis.

Accordingly, the present invention relates to a compound of the following formula (I)

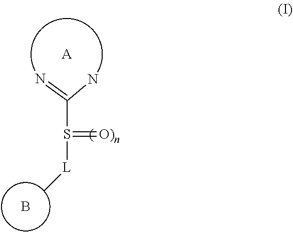

or a pharmaceutically acceptable salt or solvate thereof.

In formula (I), ring A is any one of the following groups A1 to A11:

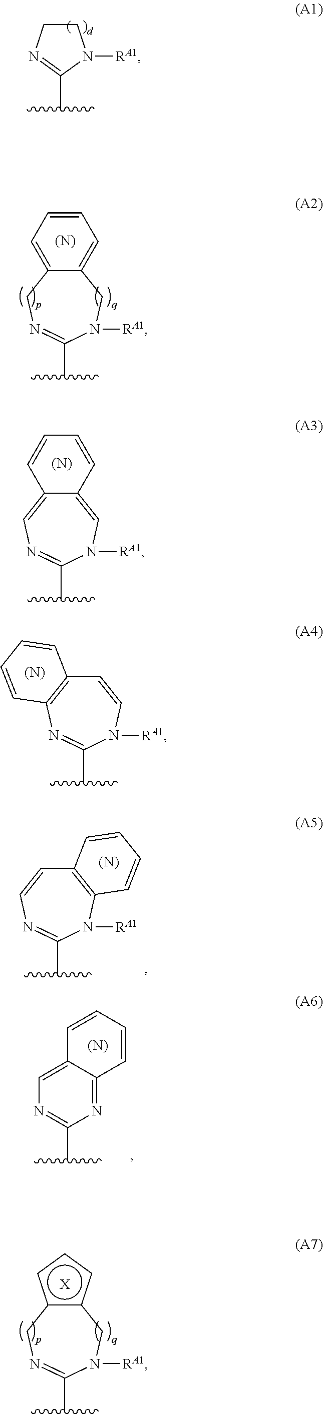

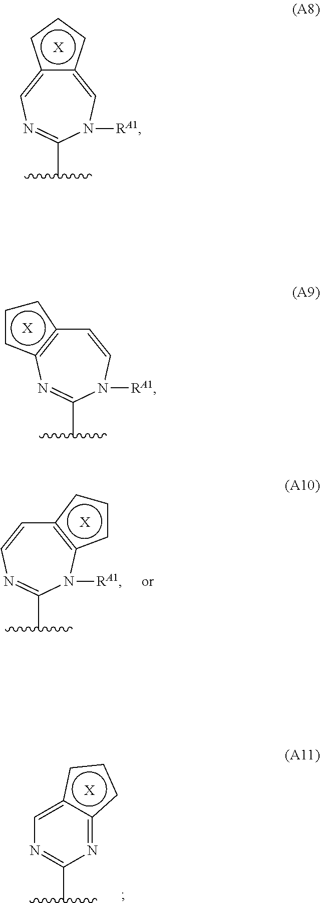

wherein d is 1, 2 or 3;
wherein p is 0, 1, 2 or 3, and q is 0, 1 or 2, with the proviso that p and q are not both 0;
wherein the symbol “(N)” depicted inside a ring indicates that 0, 1, 2 or 3 ring atom(s) of the respective ring is/are nitrogen ring atom(s);
wherein the symbol “X” depicted inside a 5-membered ring indicates that the corresponding ring is aromatic and that 1, 2 or 3 ring atom(s) of said ring is/are each independently selected from nitrogen, oxygen and sulfur, while the remaining ring atoms are carbon atoms; and
wherein ring A is optionally substituted with one or more groups R^{A2}.
n is 0, 1 or 2.
L is a covalent bond or C₁₋₅ alkylene, wherein said alkylene is optionally substituted with one or more groups R^{L}, wherein one or more —CH₂— units comprised in said alkylene are each optionally replaced by a group independently selected from —O—, —NH—, —N(C₁₋₅ alkyl)-, —CO—, —S—, —SO—, —SO₂—, carbocyclylene, and heterocyclylene, and wherein each R^{L} is independently selected from —OH, —O(C₁₋₅ alkyl), —SH, —S(C₁₋₅ alkyl), —NH₂, —NH(C₁₋₅ alkyl), —N(C₁₋₅ alkyl)(C₁₋₅ alkyl), halogen, —CF₃, —CN, C₁₋₅ alkyl, cycloalkyl, and heterocycloalkyl.

If ring A is a group A1, then ring B is selected from any one of the following groups:

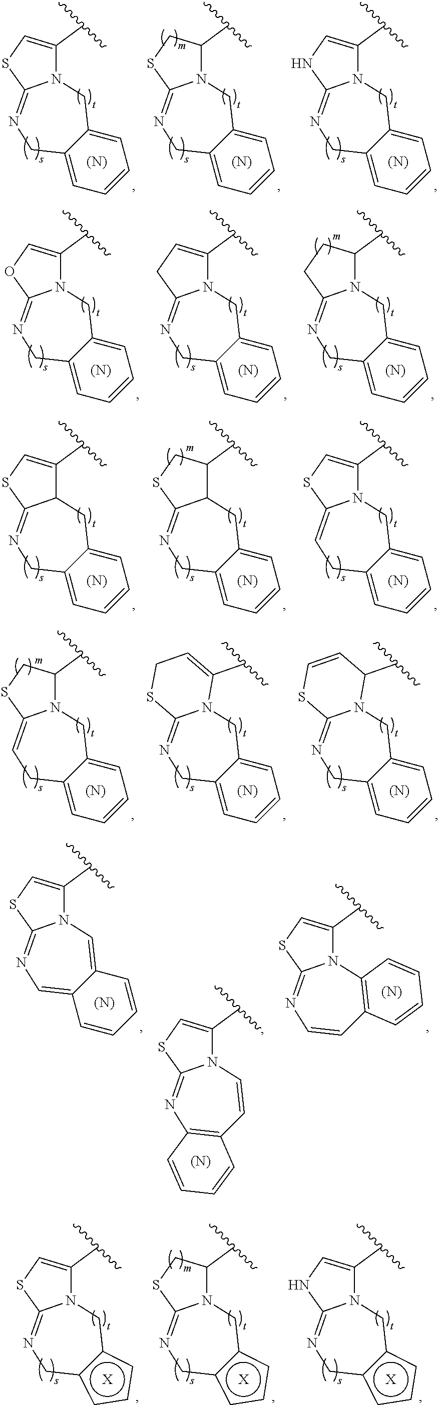

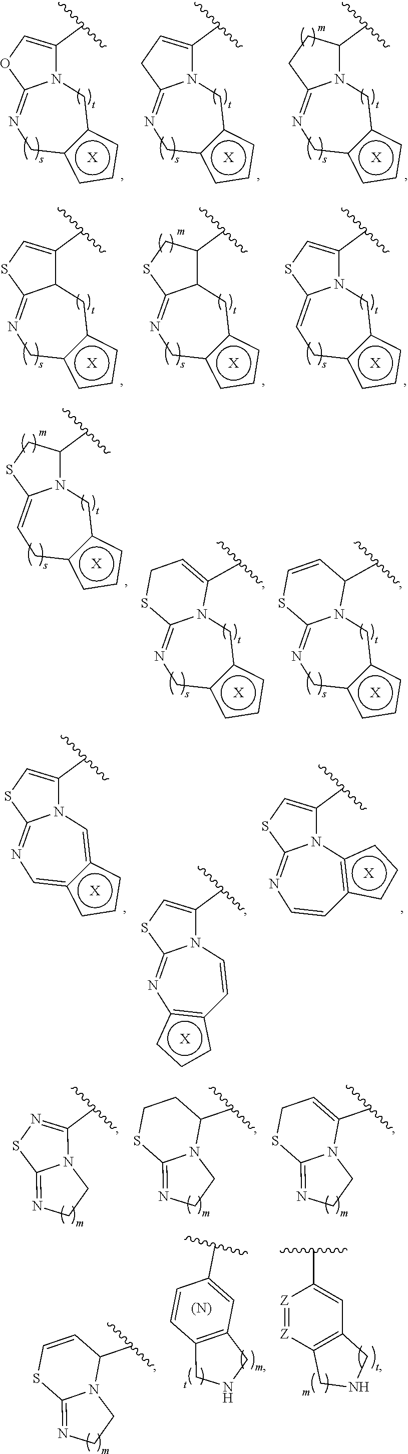

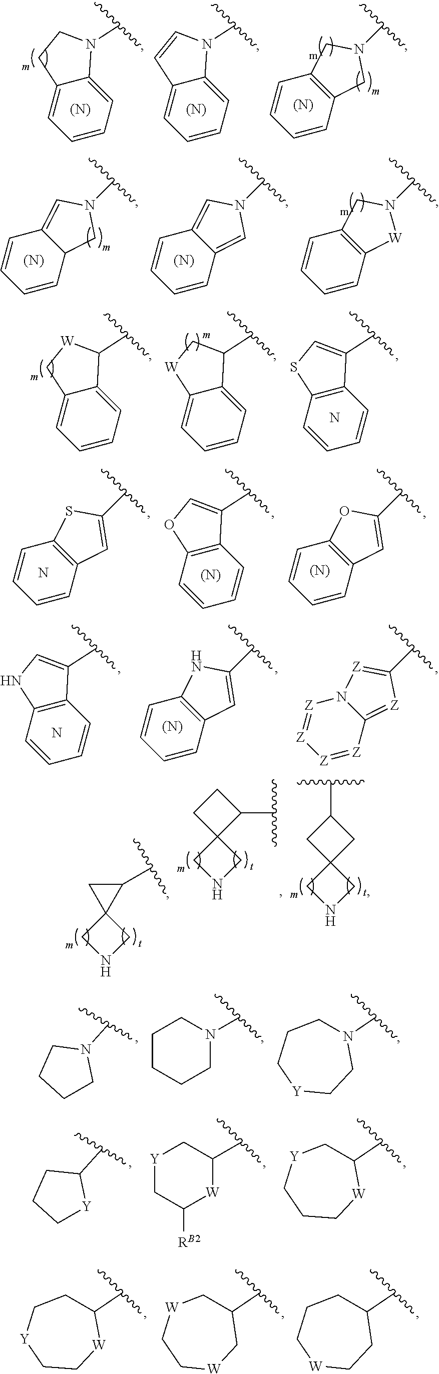

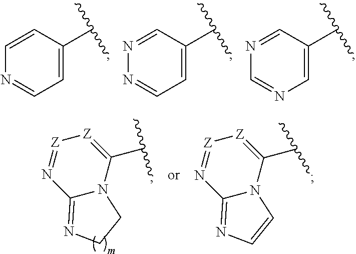

wherein each of the above-depicted groups is optionally substituted with one or more groups R^{B1};
wherein each s is independently 0, 1 or 2;
wherein each t is independently 0, 1, 2 or 3;
wherein each m is independently 1, 2 or 3;
wherein each ring atom W is independently selected from S, O, SO₂ and NH;
wherein each ring atom Y is independently selected from S, O, SO₂, NH and CH₂;
wherein each ring atom Z is independently C or N;
wherein the symbol “(N)” depicted inside a ring indicates that 0, 1, 2 or 3 ring atom(s) of the respective ring is/are nitrogen ring atom(s);
wherein the symbol “N” depicted inside a ring indicates that 1, 2 or 3 ring atom(s) of the respective ring is/are nitrogen ring atom(s); and
wherein the symbol “X” depicted inside a 5-membered ring indicates that the corresponding ring is aromatic and that 1, 2 or 3 ring atom(s) of said ring is/are each independently selected from nitrogen, oxygen and sulfur, while the remaining ring atoms are carbon atoms.

If ring A is a group A2, A3, A4, A5, A7, A8, A9 or A10, then ring B is selected from any one of the following groups:

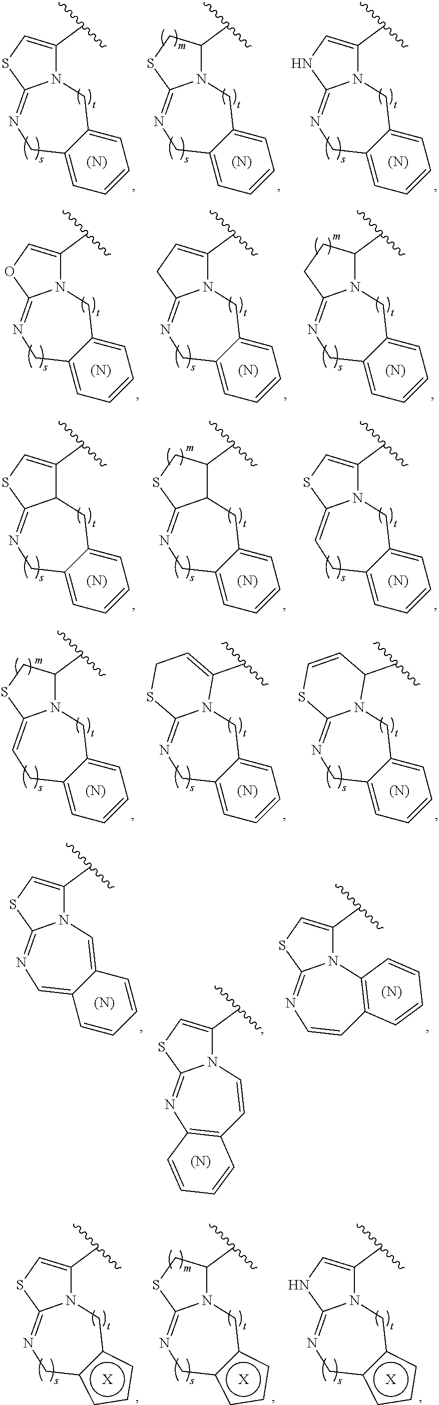

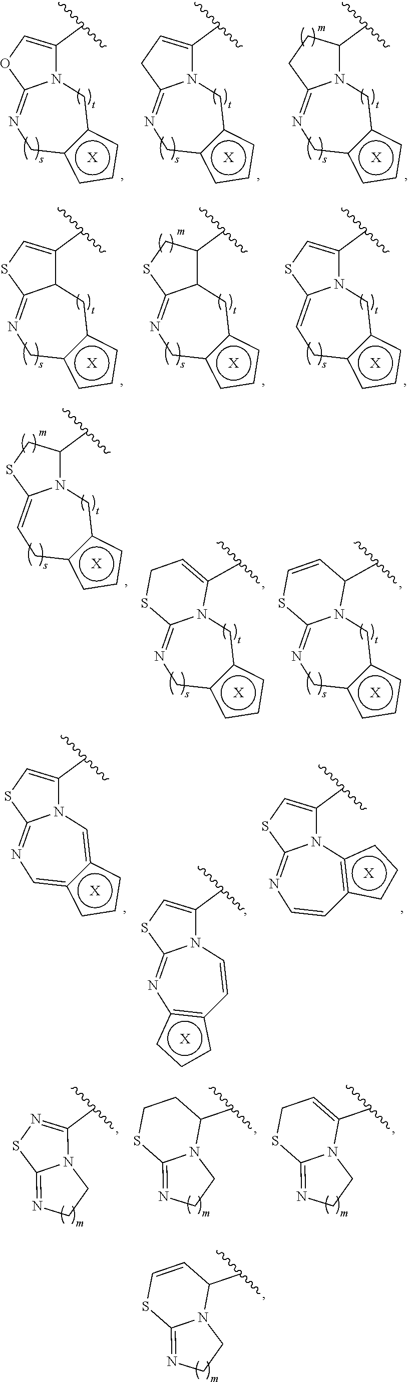

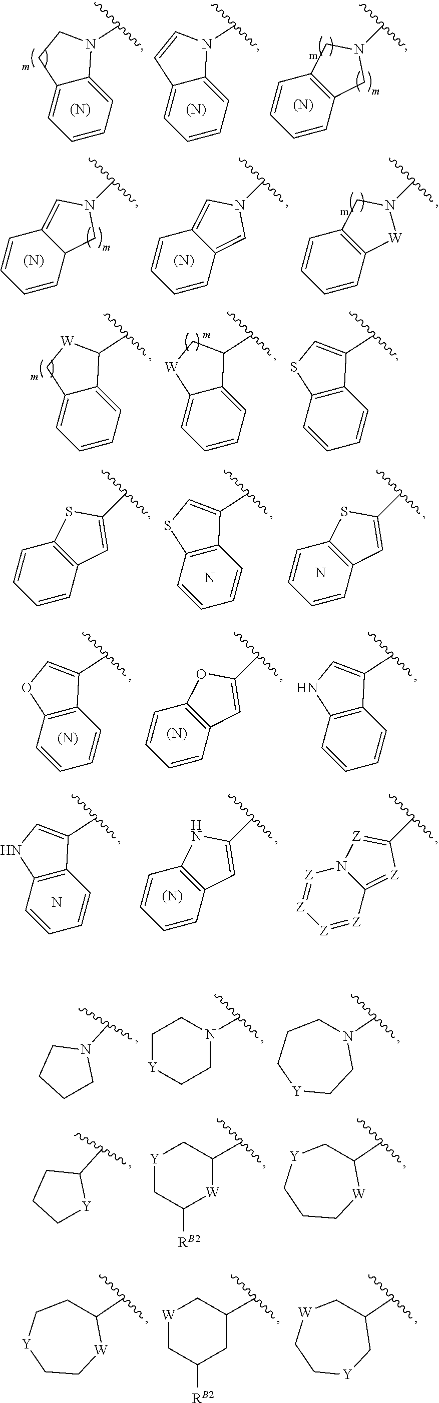

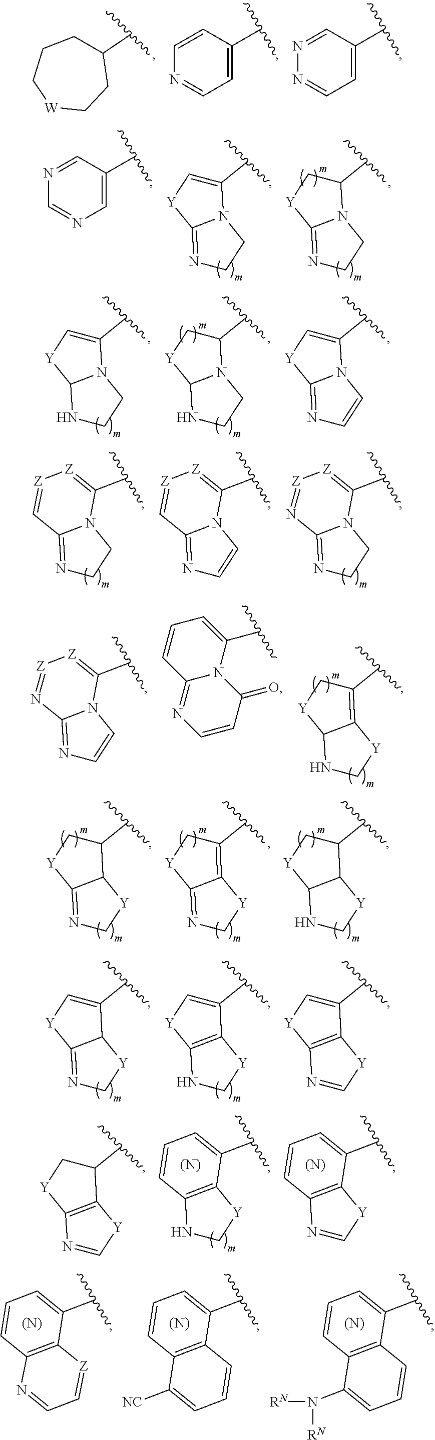

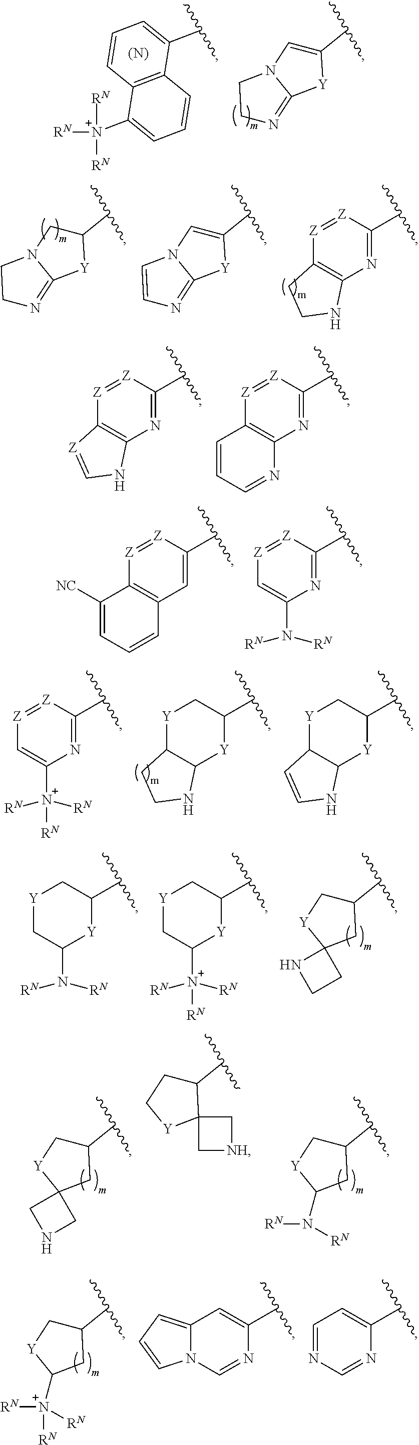

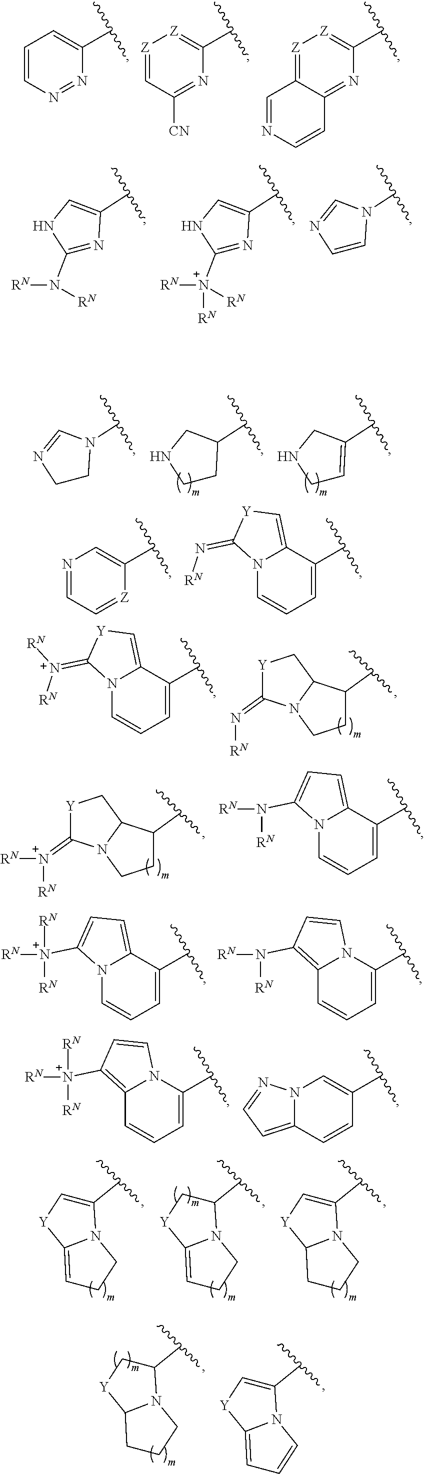

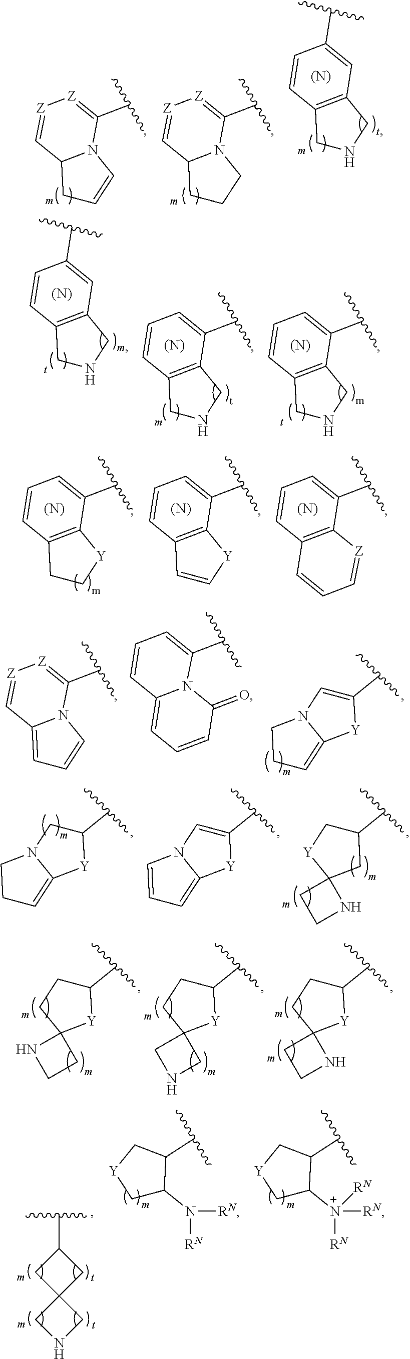

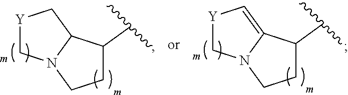

wherein each of the above-depicted groups is optionally substituted with one or more groups R^{B1};
wherein each s is independently 0, 1 or 2;
wherein each t is independently 0, 1, 2 or 3;
wherein each m is independently 1, 2 or 3;
wherein each ring atom W is independently selected from S, O, SO₂ and NH;
wherein each ring atom Y is independently selected from S, O, SO₂, NH and CH₂;
wherein each ring atom Z is independently C or N;
wherein the symbol “(N)” depicted inside a ring indicates that 0, 1, 2 or 3 ring atom(s) of the respective ring is/are nitrogen ring atom(s);
wherein the symbol “N” depicted inside a ring indicates that 1, 2 or 3 ring atom(s) of the respective ring is/are nitrogen ring atom(s); and
wherein the symbol “X” depicted inside a 5-membered ring indicates that the corresponding ring is aromatic and that 1, 2 or 3 ring atom(s) of said ring is/are each independently selected from nitrogen, oxygen and sulfur, while the remaining ring atoms are carbon atoms.

If ring A is a group A6 or A11, then ring B is selected from any one of the following groups:

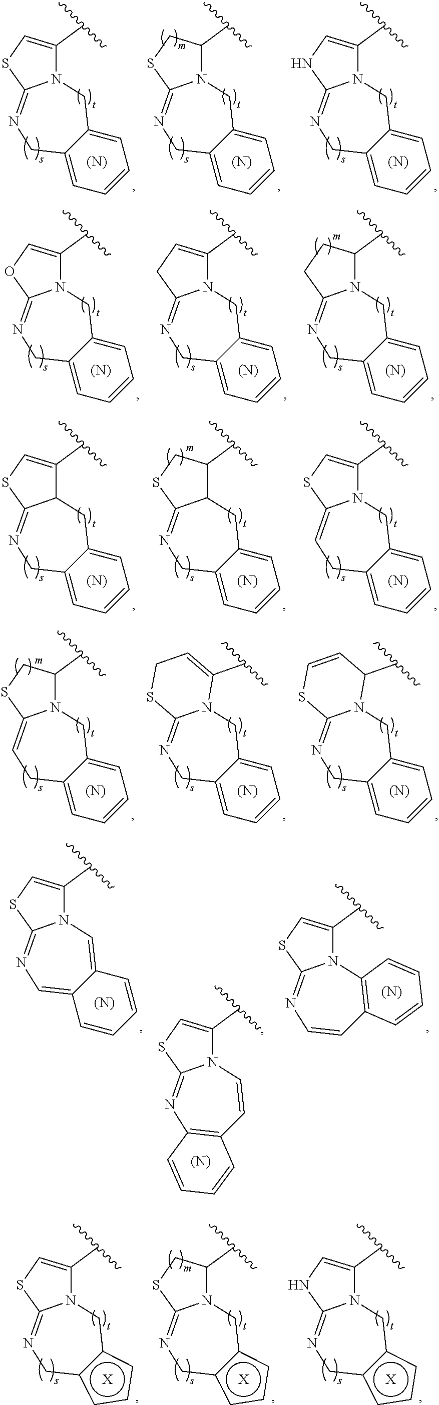

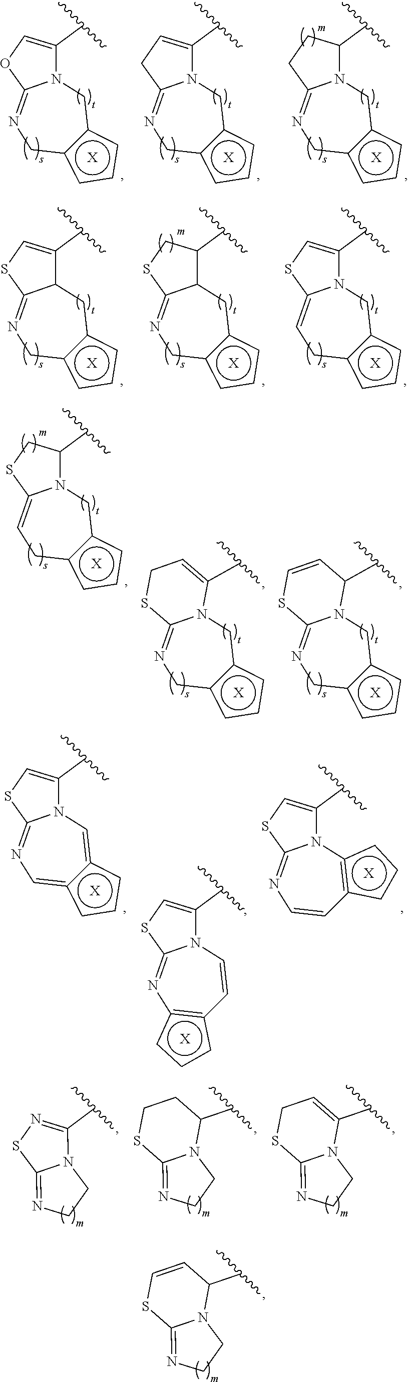

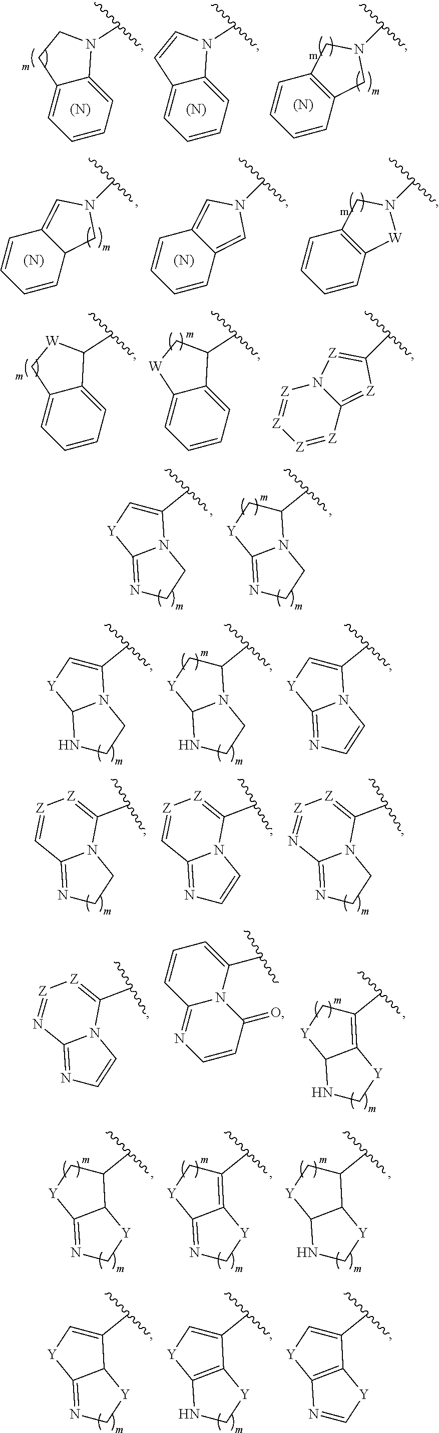

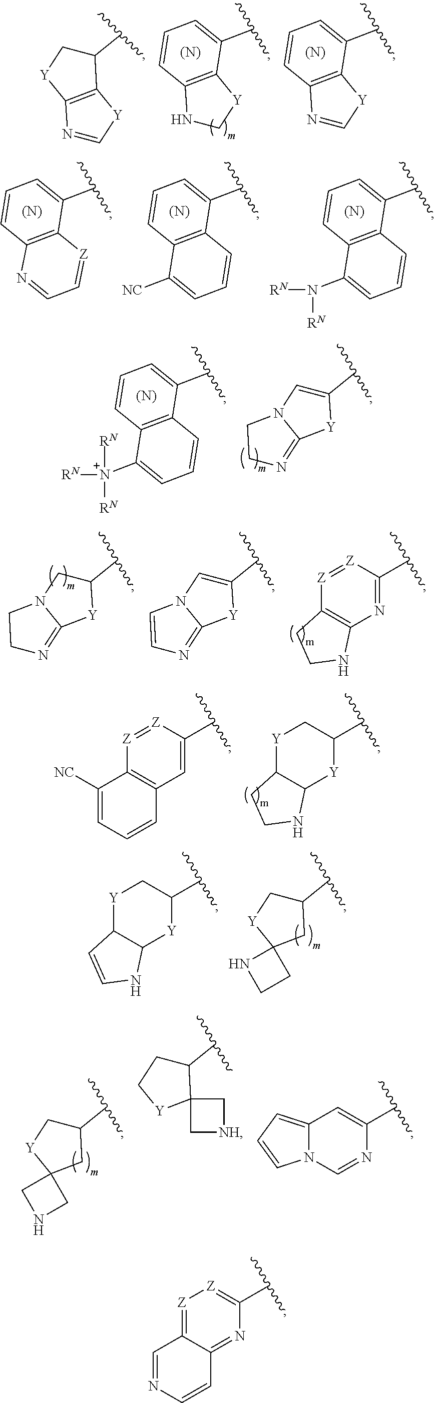

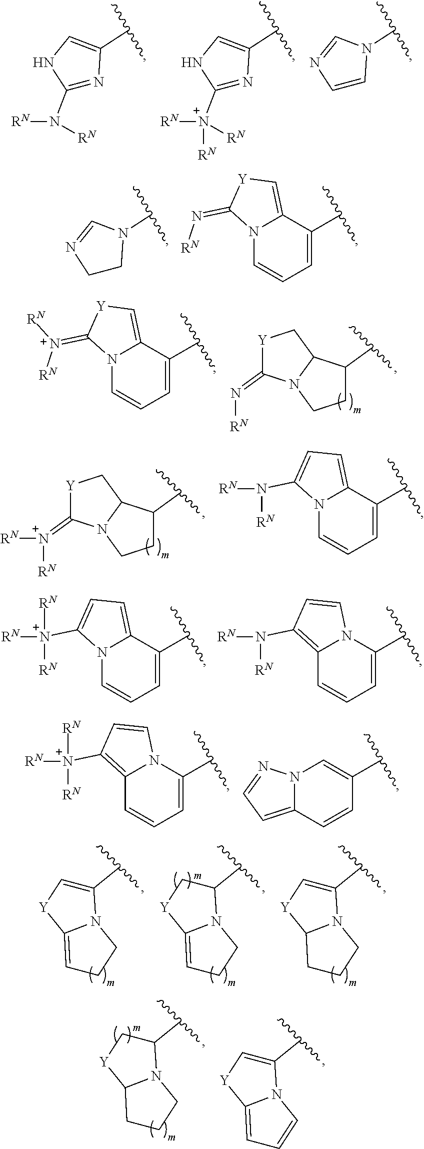

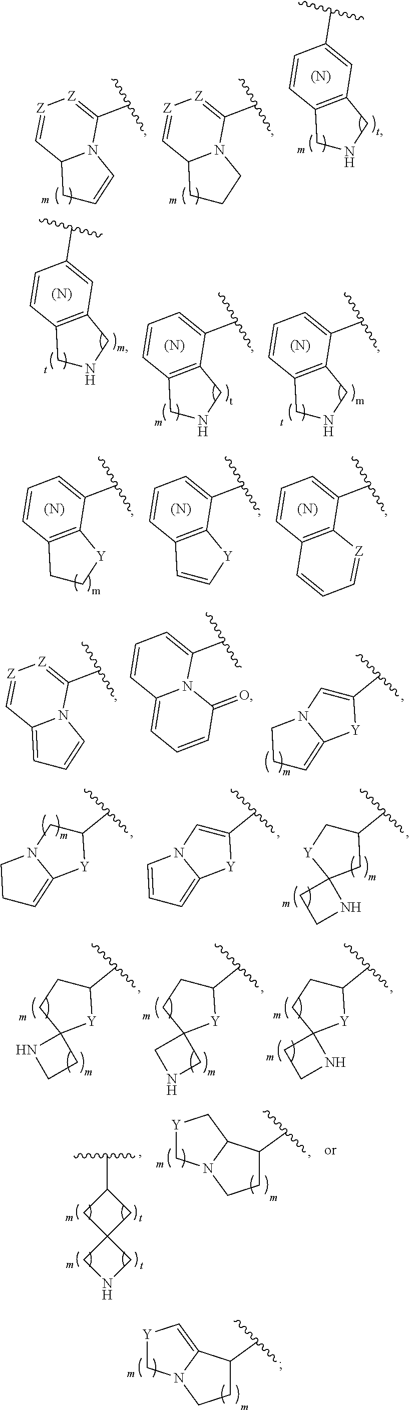

wherein each of the above-depicted groups is optionally substituted with one or more groups R^{B1};
wherein each s is independently 0, 1 or 2;
wherein each t is independently 0, 1, 2 or 3;
wherein each m is independently 1, 2 or 3;
wherein each ring atom W is independently selected from S, O, SO₂ and NH;
wherein each ring atom Y is independently selected from S, O, SO₂, NH and CH₂;
wherein each ring atom Z is independently C or N;
wherein the symbol “(N)” depicted inside a ring indicates that 0, 1, 2 or 3 ring atom(s) of the respective ring is/are nitrogen ring atom(s); and
wherein the symbol “X” depicted inside a 5-membered ring indicates that the corresponding ring is aromatic and that 1, 2 or 3 ring atom(s) of said ring is/are each independently selected from nitrogen, oxygen and sulfur, while the
remaining ring atoms are carbon atoms.

R^{A1} is selected from hydrogen, C₁₋₅ alkyl, C₂₋₅ alkenyl, C₂₋₅ alkynyl, —CO(C₁₋₅ alkyl), —COO(C₁₋₅ alkyl), carbocyclyl, and heterocyclyl, wherein said alkyl, said alkenyl, said alkynyl, the alkyl moiety in said —CO(C₁₋₅ alkyl), and the alkyl moiety in said —COO(C₁₋₅ alkyl) are each optionally substituted with one or more groups R^{Alk}, and further wherein said carbocyclyl and said heterocyclyl are each optionally substituted with one or more groups R^{Cyc}.

Each R^{A2} is independently selected from C₁₋₅ alkyl, C₂₋₅ alkenyl, C₂₋₅ alkynyl, —(C₀₋₅ alkylene)-R^{A21}, —(C₂₋₅ alkenylene)-R^{A21}, and —(C₂₋₅ alkynylene)-R^{A21}, wherein said alkyl, said alkenyl, said alkynyl, said alkylene, said alkenylene, and said alkynylene are each optionally substituted with one or more groups R^{Alk}, and further wherein one or more —CH₂— units comprised in said alkylene, said alkenylene, or said alkynylene are each optionally replaced by a group independently selected from —O—, —NH—, —N(C₁₋₅ alkyl)-, —CO—, —S—, —SO—, and —SO₂—;
wherein any two groups R^{A2}, which are attached to the same ring atom of ring A, may also be mutually joined to form, together with the ring atom that they are attached to, a cycloalkyl or a heterocycloalkyl, wherein said cycloalkyl or said heterocycloalkyl is optionally substituted with one or more groups R^{Cyc};
wherein any two groups R^{A2}, which are attached to distinct ring atoms of ring A, may also be mutually joined to form a C₁₋₅ alkylene which is optionally substituted with one or more groups R^{Cyc}, and wherein one or more —CH₂— units comprised in said alkylene are each optionally replaced by a group independently selected from —O—, —NH—, —N(C₁₋₅ alkyl)-, —CO—, —S—, —SO—, —SO₂—, and phen-1,2-diyl, wherein said phen-1,2-diyl is optionally substituted with one or more groups R^{Cyc}; and
wherein any one group R^{A2} may also be mutually joined with R^{A1} to form a C₁₋₅ alkylene which is optionally substituted with one or more groups R^{Cyc}, and wherein one or more —CH₂— units comprised in said alkylene are each optionally replaced by a group independently selected from —O—, —NH—, —N(C₁₋₅ alkyl)-, —CO—, —S—, —SO—, —SO₂—, and phen-1,2-diyl, wherein said phen-1,2-diyl is optionally substituted with one or more groups R^{Cyc}.

Each R^{A21} is independently selected from halogen, C₁₋₅ haloalkyl, —O(C₁₋₅ haloalkyl), —CN, —OR^{A22}, —NR^{A22}R^{A22}, —NR^{A22}OR^{A22}, COR^{A22}, —COOR^{A22}, —OCOR^{A22}, —CONR^{A22}R^{A22}, —NR^{A22}COR^{A22}, —NR^{A22}COOR^{A22}, —OCONR^{A22}R^{A22}, —SR^{A22}, —SOR^{A22}, —SO₂R^{A22}, —SO₂NR^{A22}R^{A22}, —NR^{A22}SO₂R^{A22}, —SO₃R^{A22}, —NO₂, carbocyclyl, and heterocyclyl, wherein said carbocyclyl and said heterocyclyl are each optionally substituted with one or more groups R^{Cyc}.

Each R^{A22} is independently selected from hydrogen, C₁₋₅ alkyl, C₂₋₅ alkenyl, C₂₋₅ alkynyl, carbocyclyl, and heterocyclyl, wherein said alkyl, said alkenyl and said alkynyl are each optionally substituted with one or more groups R^{Alk}, and further wherein said carbocyclyl and said heterocyclyl are each optionally substituted with one or more groups R^{Cyc}.

Each R^{N} is independently selected from hydrogen, C₁₋₅ alkyl, C₂₋₅ alkenyl, C₂₋₅ alkynyl, —O(C₁₋₅ alkyl), —CO(C₁₋₅ alkyl), —COO(C₁₋₅ alkyl), carbocyclyl, and heterocyclyl, wherein said alkyl, said alkenyl, said alkynyl, the alkyl moiety in said —O(C₁₋₅ alkyl), the alkyl moiety in said —CO(C₁₋₅ alkyl), and the alkyl moiety in said —COO(C₁₋₅ alkyl) are each optionally substituted with one or more groups R^{Alk}, wherein said carbocyclyl and said heterocyclyl are each optionally substituted with one or more groups R^{Cyc}, and further wherein any two groups R^{N} which are attached to the same nitrogen atom may also be mutually joined to form, together with the nitrogen atom that they are attached to, a heterocyclyl which is optionally substituted with one or more groups R^{Cyc}.

Each R^{B1} is independently selected from C₁₋₅ alkyl, C₂₋₅ alkenyl, C₂₋₅ alkynyl, —(C₀₋₅ alkylene)-R^{B11}, —(C₂₋₅ alkenylene)-R^{B11}, —(C₂₋₅ alkynylene)-R^{B11}, and ═R^{B13}, wherein said alkyl, said alkenyl, said alkynyl, said alkylene, said alkenylene, and said alkynylene are each optionally substituted with one or more groups R^{Alk}, and further wherein one or more —CH₂— units comprised in said alkylene, said alkenylene, or said alkynylene are each optionally replaced by a group independently selected from —O—, —NH—, —N(C₁₋₅ alkyl)-, —CO—, —S—, —SO—, and —SO₂—;
wherein any two groups R^{B1}, which are attached to the same ring atom of ring B, may also be mutually joined to form, together with the ring atom that they are attached to, a cycloalkyl or a heterocycloalkyl, wherein said cycloalkyl or said heterocycloalkyl is optionally substituted with one or more groups R^{Cyc}; and
wherein any two groups R^{B1}, which are attached to distinct ring atoms of ring B, may also be mutually joined to form a C₁₋₅ alkylene which is optionally substituted with one or more groups R^{Cyc}, and wherein one or more —CH₂— units comprised in said alkylene are each optionally replaced by a group independently selected from —O—, —NH—, —N(C₁₋₅ alkyl)-, —CO—, —S—, —SO—, and —SO₂—.

Each R^{B11} is independently selected from halogen, C₁₋₅ haloalkyl, —O(C₁₋₅ haloalkyl), —CN, —OR^{B12}, —NR^{B12}R^{B12}, —N⁺R^{B12}R^{B12}R^{B12}, —NR^{B12}OR^{B12}, —COR^{B12}, —COOR^{B12}, —OCOR^{B12}, —CONR^{B12}R^{B12}, —NR^{B12}COR^{B12}, —NR^{B12}COOR^{B12}, —OCONR^{B12}R^{B12}, —SR^{B12}, —SOR^{B12}, —SO₂R^{B12}, —SO₂NR^{B12}R^{B12}, —NR^{B12}SO₂R^{B12}, —SO₃R^{B12}, —NO₂, carbocyclyl, and heterocyclyl, wherein said carbocyclyl and said heterocyclyl are each optionally substituted with one or more groups R^{Cyc}.

Each R^{B12} is independently selected from hydrogen, C₁₋₅ alkyl, C₂₋₅ alkenyl, C₂₋₅ alkynyl, carbocyclyl, and heterocyclyl, wherein said alkyl, said alkenyl and said alkynyl are each optionally substituted with one or more groups R^{Alk}, and further wherein said carbocyclyl and said heterocyclyl are each optionally substituted with one or more groups R^{Cyc}.

Each R^{B13} is independently selected from ═O, ═S, and ═N—R^{B12}.

Each R^{B2} is independently selected from C₁₋₅ alkyl, C₂₋₅ alkenyl, C₂₋₅ alkynyl, —(C₀₋₅ alkylene)-R^{B21}, —(C₂₋₅ alkenylene)-R^{B21}, and —(C₂₋₅ alkynylene)-R^{B21}, wherein said alkyl, said alkenyl, said alkynyl, said alkylene, said alkenylene, and said alkynylene are each optionally substituted with one or more groups R^{Alk}, and further wherein one or more —CH₂— units comprised in said alkylene, said alkenylene, or said alkynylene are each optionally replaced by a group independently selected from —O—, —NH—, —N(C₁₋₅ alkyl)-, —CO—, —S—, —SO—, and —SO₂—.

Each R^{B21} is independently selected from halogen, C₁₋₅ haloalkyl, —O(C₁₋₅ haloalkyl), —CN, —OR^{B12}, —COR^{B12}, —COOR^{B12}, —OCOR^{B12}, —CONR^{B12}R^{B12}, —OCONR^{B12}R^{B12}, —SR^{B12}, —SOR^{B12}, —SO₂R^{B12}, —SO₂NR^{B12}R^{B12}, —NR^{B12}SO₂R^{B12}, —SO₃R^{B12}, —NO₂, carbocyclyl, and heterocyclyl, wherein said carbocyclyl and said heterocyclyl are each optionally substituted with one or more groups R^{Cyc}.

Each R^{Alk} is independently selected from —OH, —O(C₁₋₅ alkyl), —O(C₁₋₅ alkylene)-OH, —O(C₁₋₅ alkylene)-O(C₁₋₅ alkyl), —SH, —S(C₁₋₅ alkyl), —S(C₁₋₅ alkylene)-SH, —S(C₁₋₅ alkylene)-S(C₁₋₅ alkyl), —NH₂, —NH(C₁₋₅ alkyl), —N(C₁₋₅ alkyl)(C₁₋₅ alkyl), —NH—OH, —N(C₁₋₅ alkyl)-OH, —NH—O(C₁₋₅ alkyl), —N(C₁₋₅ alkyl)-O(C₁₋₅ alkyl), halogen, C₁₋₅ haloalkyl, —O(C₁₋₅ haloalkyl), —CN, —NO₂, —CHO, —CO(C₁₋₅ alkyl), —COOH, —COO(C₁₋₅ alkyl), —O—CO(C₁₋₅ alkyl), —CO—NH₂, —CO—NH(C₁₋₅ alkyl), —CO—N(C₁₋₅ alkyl)(C₁₋₅ alkyl), —NH—CO(C₁₋₅ alkyl), —N(C₁₋₅ alkyl)-CO(C₁₋₅ alkyl), —NH—COO(C₁₋₅ alkyl), —N(C₁₋₅ alkyl)-COO(C₁₋₅ alkyl), —O—CO—NH(C₁₋₅ alkyl), —O—CO—N(C₁₋₅ alkyl)(C₁₋₅ alkyl), —SO₂—NH₂, —SO₂—NH(C₁₋₅ alkyl), —SO₂—N(C₁₋₅ alkyl)(C₁₋₅ alkyl), —NH—SO₂—(C₁₋₅ alkyl), —N(C₁₋₅ alkyl)-SO₂—(C₁₋₅ alkyl), —SO₂—(C₁₋₅ alkyl), —SO—(C_{1- 5} alkyl), aryl, heteroaryl, cycloalkyl, heterocycloalkyl, and -L^{X}-R^{X}, wherein said aryl, said heteroaryl, said cycloalkyl, and said heterocycloalkyl are each optionally substituted with one or more groups independently selected from C₁₋₅ alkyl, C₂₋₅ alkenyl, C₂₋₅ alkynyl, halogen, C₁₋₅ haloalkyl, —O(C₁₋₅ haloalkyl), —CN, —OH, —O(C₁₋₅ alkyl), —SH, —S(C₁₋₅ alkyl), —NH₂, —NH(C₁₋₅ alkyl), and —N(C₁₋₅ alkyl)(C₁₋₅ alkyl).

Each R^{Cyc} is independently selected from C₁₋₅ alkyl, C₂₋₅ alkenyl, C₂₋₅ alkynyl, —OH, —O(C₁₋₅ alkyl), —O(C₁₋₅ alkylene)-OH, —O(C₁₋₅ alkylene)-O(C₁₋₅ alkyl), —SH, —S(C₁₋₅ alkyl), —S(C₁₋₅ alkylene)-SH, —S(C₁₋₅ alkylene)-S(C₁₋₅ alkyl), —NH₂, —NH(C₁₋₅ alkyl), —N(C₁₋₅ alkyl)(C₁₋₅ alkyl), —NH—OH, —N(C₁₋₅ alkyl)-OH, —NH—O(C₁₋₅ alkyl), —N(C₁₋₅ alkyl)-O(C₁₋₅ alkyl), halogen, C₁₋₅ haloalkyl, —O(C₁₋₅ haloalkyl), —CN, —NO₂, —CHO, —CO(C₁₋₅ alkyl), —COOH, —COO(C₁₋₅ alkyl), —O—CO(C₁₋₅ alkyl), —CO—NH₂, —CO—NH(C₁₋₅ alkyl), —CO—N(C₁₋₅ alkyl)(C₁₋₅ alkyl), —NH—CO(C₁₋₅ alkyl), —N(C₁₋₅ alkyl)-CO(C₁₋₅ alkyl), —NH—COO(C₁₋₅ alkyl), —N(C₁₋₅ alkyl)-COO(C₁₋₅ alkyl), —O—CO—NH(C₁₋₅ alkyl), —O—CO—N(C₁₋₅ alkyl)(C₁₋₅ alkyl), —SO₂—NH₂, —SO₂—NH(C₁₋₅ alkyl), —SO₂—N(C₁₋₅ alkyl)(C₁₋₅ alkyl), —NH—SO₂—(C₁₋₅ alkyl), —N(C₁₋₅ alkyl)-SO₂—(C₁₋₅ alkyl), —SO₂—(C₁₋₅ alkyl), —SO—(C₁₋₅ alkyl), aryl, heteroaryl, cycloalkyl, heterocycloalkyl, and -L^{X}-R^{X}, wherein said aryl, said heteroaryl, said cycloalkyl, and said heterocycloalkyl are each optionally substituted with one or more groups independently selected from C₁₋₅ alkyl, C₂₋₅ alkenyl, C₂₋₅ alkynyl, halogen, C₁₋₅ haloalkyl, —O(C₁₋₅ haloalkyl), —CN, —OH, —O(C₁₋₅ alkyl), —SH, —S(C₁₋₅ alkyl), —NH₂, —NH(C₁₋₅ alkyl), and —N(C₁₋₅ alkyl)(C₁₋₅ alkyl).

Each L^{X} is independently selected from a bond, C₁₋₅ alkylene, C₂₋₅ alkenylene, and C₂₋₅ alkynylene, wherein said alkylene, said alkenylene and said alkynylene are each optionally substituted with one or more groups independently selected from halogen, C₁₋₅ haloalkyl, —CN, —OH, —O(C₁₋₅ alkyl), —SH, —S(C₁₋₅ alkyl), —NH₂, —NH(C₁₋₅ alkyl), and —N(C₁₋₅ alkyl)(C₁₋₅ alkyl), and further wherein one or more —CH₂— units comprised in said alkylene, said alkenylene or said alkynylene are each optionally replaced by a group independently selected from —O—, —NH—, —N(C₁₋₅ alkyl)-, —CO—, —S—, —SO—, and —SO₂—.

Each R^{X} is independently selected from —OH, —O(C₁₋₅ alkyl), —O(C₁₋₅ alkylene)-OH, —O(C₁₋₅ alkylene)-O(C₁₋₅ alkyl), —SH, —S(C₁₋₅ alkyl), —S(C₁₋₅ alkylene)-SH, —S(C₁₋₅ alkylene)-S(C₁₋₅ alkyl), —NH₂, —NH(C₁₋₅ alkyl), —N(C₁₋₅ alkyl)(C₁₋₅ alkyl), —NH—OH, —N(C₁₋₅ alkyl)-OH, —NH—O(C₁₋₅ alkyl), —N(C₁₋₅ alkyl)-O(C₁₋₅ alkyl), halogen, C₁₋₅ haloalkyl, —O(C₁₋₅ haloalkyl), —CN, —NO₂, —CHO, —CO(C₁₋₅ alkyl), —COOH, —COO(C₁₋₅ alkyl), —O—CO(C₁₋₅ alkyl), —CO—NH₂, —CO—NH(C₁₋₅ alkyl), —CO—N(C₁₋₅ alkyl)(C₁₋₅ alkyl), —NH—CO(C₁₋₅ alkyl), —N(C₁₋₅ alkyl)-CO(C₁₋₅ alkyl), —NH—COO(C₁₋₅ alkyl), —N(C₁₋₅ alkyl)-COO(C₁₋₅ alkyl), —O—CO—NH(C₁₋₅ alkyl), —O—CO—N(C₁₋₅ alkyl)(C₁₋₅ alkyl), —SO₂—NH₂, —SO₂—NH(C₁₋₅ alkyl), —SO₂—N(C₁₋₅ alkyl)(C₁₋₅ alkyl), —NH—SO₂—(C₁₋₅ alkyl), —N(C₁₋₅ alkyl)-SO₂—(C₁₋₅ alkyl), —SO₂—(C₁₋₅ alkyl), —SO—(C_{1- 5} alkyl), aryl, heteroaryl, cycloalkyl, and heterocycloalkyl, wherein said aryl, said heteroaryl, said cycloalkyl, and said heterocycloalkyl are each optionally substituted with one or more groups independently selected from C₁₋₅ alkyl, C₂₋₅ alkenyl, C₂₋₅ alkynyl, halogen, C₁₋₅ haloalkyl, —O(C₁₋₅ haloalkyl), —CN, —OH, —O(C₁₋₅ alkyl), —SH, —S(C₁₋₅ alkyl), —NH₂, —NH(C₁₋₅ alkyl), and —N(C₁₋₅ alkyl)(C₁₋₅ alkyl).

In accordance with the present invention, the following compounds are excluded from formula (I):
1-(((5-phenyl-4,5-dihydro-1H-imidazol-2-yl)thio)methyl)pyrrolidine;
1-(2-((5-phenyl-4,5-dihydro-1H-imidazol-2-yl)thio)ethyl)pyrrolidine;
1-(3-((5-phenyl-4,5-dihydro-1H-imidazol-2-yl)thio)propyl)pyrrolidine;
1-(((5-phenyl-4,5-dihydro-1H-imidazol-2-yl)thio)methyl)piperidine;
1-(2-((5-phenyl-4,5-dihydro-1H-imidazol-2-yl)thio)ethyl)piperidine;
1-(3-((5-phenyl-4,5-dihydro-1H-imidazol-2-yl)thio)propyl)piperidine;
1-(2-((4,5-dihydro-1H-imidazol-2-yl)thio)ethyl)pyrrolidine;
1-(3-((4,5-dihydro-1H-imidazol-2-yl)thio)propyl)pyrrolidine;
1-(4-((4,5-dihydro-1H-imidazol-2-yl)thio)butyl)pyrrolidine;
1-(2-((4,5-dihydro-1H-imidazol-2-yl)thio)ethyl)piperidine;
1-(3-((4,5-dihydro-1H-imidazol-2-yl)thio)propyl)piperidine;
1-(4-((4,5-dihydro-1H-imidazol-2-yl)thio)butyl)piperidine;
1-(2-((4,5-dihydro-1H-imidazol-2-yl)thio)ethyl)azepane;
1-(3-((4,5-dihydro-1H-imidazol-2-yl)thio)propyl)azepane;
1-(4-((4,5-dihydro-1H-imidazol-2-yl)thio)butyl)azepane;
1-(2-((1,4,5,6-tetrahydropyrimidin-2-yl)thio)ethyl)pyrrolidine;
1-(3-((1,4,5,6-tetrahydropyrimidin-2-yl)thio)propyl)pyrrolidine;
1-(4-((1,4,5,6-tetrahydropyrimidin-2-yl)thio)butyl)pyrrolidine;
1-(2-((1,4,5,6-tetrahydropyrimidin-2-yl)thio)ethyl)piperidine;
1-(3-((1,4,5,6-tetrahydropyrimidin-2-yl)thio)propyl)piperidine;
1-(4-((1,4,5,6-tetrahydropyrimidin-2-yl)thio)butyl)piperidine;
1-(2-((1,4,5,6-tetrahydropyrimidin-2-yl)thio)ethyl)azepane;
1-(3-((1,4,5,6-tetrahydropyrimidin-2-yl)thio)propyl)azepane;
1-(4-((1,4,5,6-tetrahydropyrimidin-2-yl)thio)butyl)azepane;
1-(2-((4,5,6,7-tetrahydro-1H-1,3-diazepin-2-yl)thio)ethyl)pyrrolidine;
1-(3-((4,5,6,7-tetrahydro-1H-1,3-diazepin-2-yl)thio)propyl)pyrrolidine;
1-(4-((4,5,6,7-tetrahydro-1H-1,3-diazepin-2-yl)thio)butyl)pyrrolidine;
1-(2-((4,5,6,7-tetrahydro-1H-1,3-diazepin-2-yl)thio)ethyl)piperidine;
1-(3-((4,5,6,7-tetrahydro-1H-1,3-diazepin-2-yl)thio)propyl)piperidine;
1-(4-((4,5,6,7-tetrahydro-1H-1,3-diazepin-2-yl)thio)butyl)piperidine;
1-(2-((4,5,6,7-tetrahydro-1H-1,3-diazepin-2-yl)thio)ethyl)azepane;
1-(3-((4,5,6,7-tetrahydro-1H-1,3-diazepin-2-yl)thio)propyl)azepane;
1-(4-((4,5,6,7-tetrahydro-1H-1,3-diazepin-2-yl)thio)butyl)azepane;
2-((4,5-dihydro-1H-imidazol-2-yl)thio)-1-(pyridin-4-yl)ethan-1-one;
3-(1-(3-((4,5-dihydro-1H-imidazol-2-yl)thio)propyl)-1H-indol-3-yl)-4-(1-methyl-1H-indol-3-yl)-1H-pyrrole-2,5-dione;
2-((4,5-dihydro-1H-imidazol-2-yl)thio)-1-(pyridin-4-yl)ethanone;
3-((1,4,5,6-tetrahydropyrimidin-2-yl)thio)-1H-pyrrolo[2,3-b]pyridine; and
3-((3,4-dihydroquinazolin-2-yl)thio)-1H-indole-2-carboxylic acid.

The following compounds are preferably also excluded from formula (I):
2-(cyclopentylthio)-4,5-dihydro-1H-imidazole;
N-(piperidinomethyl)-2-[(piperidinomethyl)thio]-2-imidazoline;
N-((2-methylpiperidino)methyl)-2-[((2-methylpiperidino)methyl)thio]-2-imidazoline;
N-((3-methylpiperidino)methyl)-2-[((3-methylpiperidino)methyl)thio]-2-imidazoline;
N-((4-methylpiperidino)methyl)-2-[((4-methylpiperidino)methyl)thio]-2-imidazoline; and
N-((2-methyl-5-ethylpiperidino)methyl)-2-[((2-methyl-5-ethylpiperidino)methyl)thio]-2-imidazoline.

The present invention also relates to a pharmaceutical composition comprising a compound of formula (I), or a pharmaceutically acceptable salt or solvate thereof, in combination with a pharmaceutically acceptable excipient. Accordingly, the invention relates to a compound of formula (I) or a pharmaceutically acceptable salt or solvate thereof, or a pharmaceutical composition comprising any of the aforementioned entities and a pharmaceutically acceptable excipient, for use as a medicament.

The invention further relates to a compound of formula (I) or a pharmaceutically acceptable salt or solvate thereof, or a pharmaceutical composition comprising any of the aforementioned entities and a pharmaceutically acceptable excipient, for use in the treatment or prevention of an inflammatory disorder, an autoimmune disorder, an autoinflammatory disorder, or an interferonopathy.

Moreover, the present invention relates to the use of a compound of formula (I) or a pharmaceutically acceptable salt or solvate thereof in the preparation of a medicament for the treatment or prevention of an inflammatory disorder, an autoimmune disorder, an autoinflammatory disorder, or an interferonopathy.

The invention likewise relates to a method of treating or preventing an inflammatory disorder, an autoimmune disorder, an autoinflammatory disorder, or an interferonopathy, the method comprising administering a compound of formula (I) or a pharmaceutically acceptable salt or solvate thereof, or a pharmaceutical composition comprising any of the aforementioned entities in combination with a pharmaceutically acceptable excipient, to a subject (preferably a human) in need thereof. It will be understood that a therapeutically effective amount of the compound of formula (I) or the pharmaceutically acceptable salt or solvate thereof (or of the pharmaceutical composition) is to be administered in accordance with this method.

The diseases/disorders to be treated or prevented in accordance with the present invention, i.e. the inflammatory disorders, autoimmune disorders, autoinflammatory disorders and interferonopathies, include in particular a rheumatologic inflammatory disorder, a skin inflammatory disorder, a lung inflammatory disorder, a muscle inflammatory disorder, a bowel inflammatory disorder, a brain inflammatory disorder, an autoimmune disorder, an autoinflammatory disorder, or a type I interferonopathy.

The interferonopathy (or type I interferonopathy) to be treated or prevented in accordance with the invention may be, e.g., a monogenic interferonopathy (particularly a monogenic type I interferonopathy). Preferably, the interferonopathy (or type I interferonopathy) to be treated or prevented is selected from Aicardi-Goutieres syndrome, familial chilblain lupus, Singleton-Merten syndrome, proteasome-associated autoinflammatory syndrome, deficiency of adenosine deaminase 2, retinal vasculopathy with cerebral leukodystrophy, STING-associated vasculopathy with onset in infancy, spondyloenchondrodysplasia (e.g., spondyloenchondrodysplasia with immune dysregulation), systemic lupus erythematosus (SLE), ISG15 deficiency, or an interferonopathy associated with genetic dysfunction (e.g., an interferonopathy associated with DNASEII deficiency, proteasome deficiency (CANDLE/PRAAS), TREX1 deficiency, IFIH1 gain of function (GOF), STING GOF, DDX58 GOF, CECR1 deficiency, ADAR1 deficiency, RNASEH2 deficiency, RNASET2 deficiency, DNASE1L3 deficiency, complement deficiency (C1Q, C3 and/or C4), ACP5 deficiency, or SAMHD1 deficiency).

The inflammatory disorder, autoimmune disorder or autoinflammatory disorder is preferably selected from familial Mediterranean fever, TNF receptor associated periodic fever syndrome, periodic fever, aphthous stomatitis, pharyngitis, cervical adenitis, pyogenic arthritis, pyoderma gangrenosum, acne, Blau syndrome, neonatal onset multisystem inflammatory disease, familial cold autoinflammatory syndrome, hyperimmunoglobulinemia D with periodic fever syndrome, Muckle-Wells syndrome, chronic infantile neurological cutaneous and articular syndrome, deficiency of interleukin-1 receptor antagonist, haploinsufficiency of A20, deficiency of IL-36 receptor antagonist, CARD14-mediated psoriasis, inflammatory bowel disease (e.g., early-onset inflammatory bowel disease), PLCG2-associated autoinflammation, antibody deficiency and immune dysregulation, an inflammatory disorder associated with genetic dysfunction (e.g., an inflammatory disorder associated with MEFV deficiency, MEFV gain of function (GOF), MFV deficiency, TNFRSF1A GOF, NOD2 GOF, NLRP3 GOF, PSTPIP1 GOF, A20 LOF, IL36RN deficiency, CARD14 GOF, NLRC4 GOF, IL10 RA/RB deficiency, IL-10 deficiency, NOD2 GOF, or PLCG2 GOF), rheumatoid arthritis, spondyloarthritis, osteoarthritis, gout, idiopathic juvenile arthritis, psoriatic arthritis, eczema, psoriasis, scleroderma, systemic lupus erythematosus, Sjögren's syndrome, dermatomyositis, overlapping myositis, mixed connective tissue disease, undifferentiated connective tissue disease, chronic obstructive pulmonary disease, bowel inflammation, Crohn disease, Behcçet's disease, ulcerative colitis, sepsis, macrophages activation syndrome, acute respiratory distress syndrome, type II diabetes, asthma, chronic wounds, autism, multiple sclerosis, Alzheimer's disease, Parkinson's disease, chronic inflammatory demyelinating polyneuropathy, juvenile dermatomyositis, or an inflammatory complication associated with a viral infection (e.g., an inflammatory complication associated with Ebola, dengue fever, measles, or meningitis).

Accordingly, the present invention relates, in particular, to the compound of formula (I) or a pharmaceutically acceptable salt or solvate thereof for use in treating or preventing any of the following diseases/disorders: a rheumatologic inflammatory disorder, a skin inflammatory disorder, a lung inflammatory disorder, a muscle inflammatory disorder, a bowel inflammatory disorder, a brain inflammatory disorder, an autoinflammatory disorder, an autoimmune disorder, a type I interferonopathy, Aicardi-Goutières syndrome, familial chilblain lupus, Singleton-Merten syndrome, proteasome-associated autoinflammatory syndrome, deficiency of adenosine deaminase 2, retinal vasculopathy with cerebral leukodystrophy, STING-associated vasculopathy with onset in infancy, spondyloenchondrodysplasia (e.g., spondyloenchondrodysplasia with immune dysregulation), ISG15 deficiency, an interferonopathy associated with genetic dysfunction (e.g., an interferonopathy associated with DNASEII deficiency, proteasome deficiency (CANDLE/PRAAS), TREX1 deficiency, IFIH1 gain of function (GOF), STING GOF, DDX58 GOF, CECR1 deficiency, ADAR1 deficiency, RNASEH2 deficiency, RNASET2 deficiency, DNASE1L3 deficiency, complement deficiency (C1Q, C3 and/or C4), ACP5 deficiency, or SAMHD1 deficiency), familial Mediterranean fever, TNF receptor associated periodic fever syndrome, periodic fever, aphthous stomatitis, pharyngitis, cervical adenitis, pyogenic arthritis, pyoderma gangrenosum, acne, Blau syndrome, neonatal onset multisystem inflammatory disease, familial cold autoinflammatory syndrome, hyperimmunoglobulinemia D with periodic fever syndrome, Muckle-Wells syndrome, chronic infantile neurological cutaneous and articular syndrome, deficiency of interleukin-1 receptor antagonist, haploinsufficiency of A20, deficiency of IL-36 receptor antagonist, CARD14-mediated psoriasis, inflammatory bowel disease (e.g., early-onset inflammatory bowel disease), PLCG2-associated autoinflammation, antibody deficiency and immune dysregulation, an inflammatory disorder associated with genetic dysfunction (e.g., an inflammatory disorder associated with MEFV deficiency, MEFV gain of function (GOF), MFV deficiency, TNFRSF1A GOF, NOD2 GOF, NLRP3 GOF, PSTPIP1 GOF, A20 LOF, IL36RN deficiency, CARD14 GOF, NLRC4 GOF, IL10 RA/RB deficiency, IL-10 deficiency, NOD2 GOF, or PLCG2 GOF), rheumatoid arthritis, spondyloarthritis, osteoarthritis, gout, idiopathic juvenile arthritis, psoriatic arthritis, eczema, psoriasis, scleroderma, systemic lupus erythematosus, Sjögren's syndrome, dermatomyositis, overlapping myositis, mixed connective tissue disease, undifferentiated connective tissue disease, chronic obstructive pulmonary disease, bowel inflammation, Crohn disease, Behcçet's disease, ulcerative colitis, sepsis, macrophages activation syndrome, acute respiratory distress syndrome, type II diabetes, asthma, chronic wounds, autism, multiple sclerosis, Alzheimer's disease, Parkinson's disease, chronic inflammatory demyelinating polyneuropathy, juvenile dermatomyositis, or an inflammatory complication associated with a viral infection (e.g., an inflammatory complication associated with Ebola, dengue fever, measles, or meningitis).

Preferably, the invention relates to the compound of formula (I) or a pharmaceutically acceptable salt or solvate thereof for use in treating or preventing any of the following diseases/disorders: Aicardi-Goutières syndrome, familial chilblain lupus, Singleton-Merten syndrome, proteasome-associated autoinflammatory syndrome, deficiency of adenosine deaminase 2, retinal vasculopathy with cerebral leukodystrophy, STING-associated vasculopathy with onset in infancy, spondyloenchondrodysplasia (e.g., spondyloenchondrodysplasia with immune dysregulation), ISG15 deficiency, an interferonopathy associated with genetic dysfunction (e.g., an interferonopathy associated with DNASEII deficiency, proteasome deficiency (CANDLE/PRAAS), TREX1 deficiency, IFIH1 gain of function (GOF), STING GOF, DDX58 GOF, CECR1 deficiency, ADAR1 deficiency, RNASEH2 deficiency, RNASET2 deficiency, DNASE1L3 deficiency, complement deficiency (C1Q, C3 and/or C4), ACP5 deficiency, or SAMHD1 deficiency), familial Mediterranean fever, TNF receptor associated periodic fever syndrome, periodic fever, aphthous stomatitis, pharyngitis, cervical adenitis, pyogenic arthritis, pyoderma gangrenosum, acne, Blau syndrome, neonatal onset multisystem inflammatory disease, familial cold autoinflammatory syndrome, hyperimmunoglobulinemia D with periodic fever syndrome, Muckle-Wells syndrome, chronic infantile neurological cutaneous and articular syndrome, deficiency of interleukin-1 receptor antagonist, haploinsufficiency of A20, deficiency of IL-36 receptor antagonist, CARD14-mediated psoriasis, inflammatory bowel disease (e.g., early-onset inflammatory bowel disease), PLCG2-associated autoinflammation, antibody deficiency and immune dysregulation, an inflammatory disorder associated with genetic dysfunction (e.g., an inflammatory disorder associated with MEFV deficiency, MEFV gain of function (GOF), MFV deficiency, TNFRSF1A GOF, NOD2 GOF, NLRP3 GOF, PSTPIP1 GOF, A20 LOF, IL36RN deficiency, CARD14 GOF, NLRC4 GOF, IL10 RA/RB deficiency, IL-10 deficiency, NOD2 GOF, or PLCG2 GOF), rheumatoid arthritis, spondyloarthritis, osteoarthritis, gout, idiopathic juvenile arthritis, psoriatic arthritis, eczema, psoriasis, scleroderma, systemic lupus erythematosus, Sjögren's syndrome, dermatomyositis, overlapping myositis, mixed connective tissue disease, undifferentiated connective tissue disease, chronic obstructive pulmonary disease, bowel inflammation, Crohn disease, Behçet's disease, ulcerative colitis, sepsis, macrophages activation syndrome, acute respiratory distress syndrome, type II diabetes, asthma, chronic wounds, autism, multiple sclerosis, Alzheimer's disease, Parkinson's disease, chronic inflammatory demyelinating polyneuropathy, juvenile dermatomyositis, or an inflammatory complication associated with a viral infection (e.g., an inflammatory complication associated with Ebola, dengue fever, measles, or meningitis).

More preferably, the present invention relates to the compound of formula (I) or a pharmaceutically acceptable salt or solvate thereof for use in treating or preventing rheumatoid arthritis, dermatomyositis (e.g., juvenile dermatomyositis), or systemic lupus erythematosus.

The present invention furthermore relates to the use of a compound of formula (I) or a pharmaceutically acceptable salt or solvate thereof as a C—X—C chemokine receptor type 4 (CXCR4) modulator in research, particularly as a research tool compound for modulating CXCR4. Accordingly, the invention refers to the in vitro use of a compound of formula (I) or a pharmaceutically acceptable salt or solvate thereof as a CXCR4 modulator and, in particular, to the in vitro use of a compound of formula (I) or a pharmaceutically acceptable salt or solvate thereof as a research tool compound acting as a CXCR4 modulator. The invention likewise relates to a method, particularly an in vitro method, of modulating CXCR4, the method comprising the application of a compound of formula (I) or a pharmaceutically acceptable salt or solvate thereof. The invention further relates to a method of modulating CXCR4, the method comprising applying a compound of formula (I) or a pharmaceutically acceptable salt or solvate thereof to a test sample (e.g., a biological sample) or a test animal (i.e., a non-human test animal). The invention also refers to a method, particularly an in vitro method, of modulating CXCR4 in a sample (e.g., a biological sample), the method comprising applying a compound of formula (I) or a pharmaceutically acceptable salt or solvate thereof to said sample. The present invention further provides a method of modulating CXCR4, the method comprising contacting a test sample (e.g., a biological sample) or a test animal (i.e., a non-human test animal) with a compound of formula (I) or a pharmaceutically acceptable salt or solvate thereof. The terms “sample”, “test sample” and “biological sample” include, without being limited thereto: a cell, a cell culture or a cellular or subcellular extract; biopsied material obtained from an animal (e.g., a human), or an extract thereof; or blood, serum, plasma, saliva, urine, feces, or any other body fluid, or an extract thereof. It is to be understood that the term “in vitro” is used in this specific context in the sense of “outside a living human or animal body”, which includes, in particular, experiments performed with cells, cellular or subcellular extracts, and/or biological molecules in an artificial environment such as an aqueous solution or a culture medium which may be provided, e.g., in a flask, a test tube, a Petri dish, a microtiter plate, etc.

The compound of formula (I) as well as the pharmaceutically acceptable salts and solvates thereof will be described in more detail in the following.

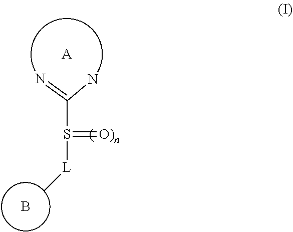

In formula (I), ring A is any one of the following groups A1 to A11:

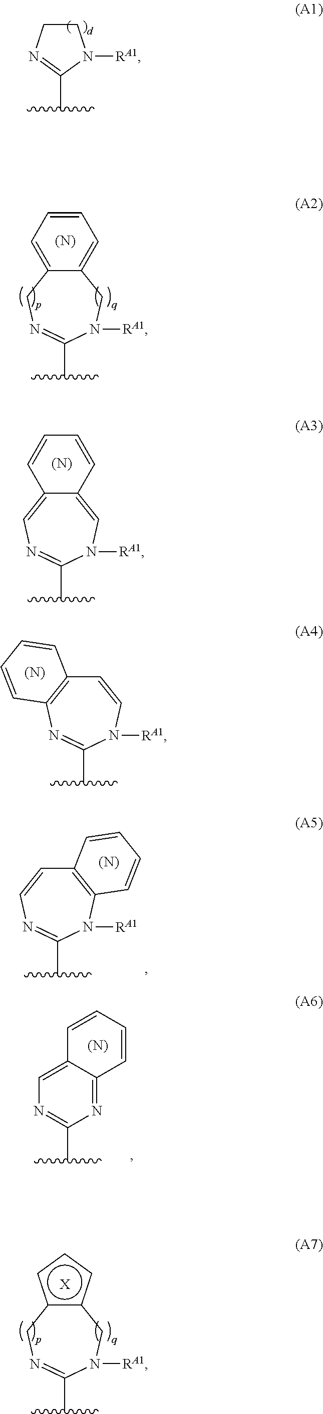

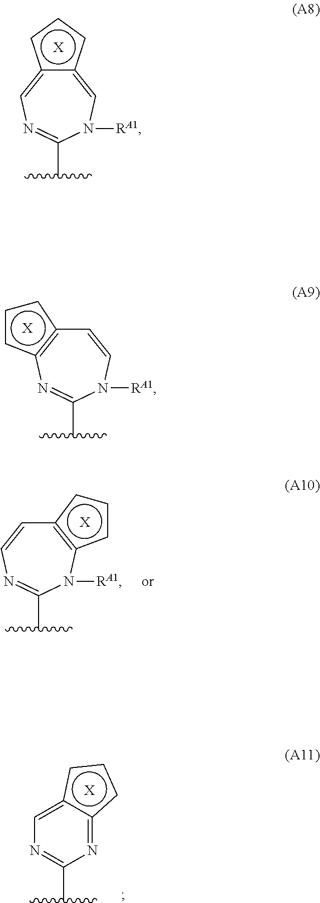

wherein d is 1, 2 or 3;
wherein p is 0, 1, 2 or 3, and q is 0, 1 or 2, with the proviso that p and q are not both 0;
wherein the symbol “(N)” depicted inside a ring (e.g., as in

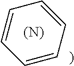

indicates that 0, 1, 2 or 3 ring atom(s) of the respective ring is/are nitrogen ring atom(s);
wherein the symbol “X” depicted inside a 5-membered ring indicates that the corresponding ring is aromatic and that 1, 2 or 3 ring atom(s) of said ring is/are each independently selected from nitrogen, oxygen and sulfur, while the remaining ring atoms are carbon atoms; and
wherein ring A is optionally substituted with one or more (e.g., one, two or three) groups R^{A2}.

In the case of the above-depicted group A1, the variable d may be 1, 2 or 3. The corresponding group A1 will thus have the following structure A1a (if d=1), A1b (if d=2), or A1c (if d=3):

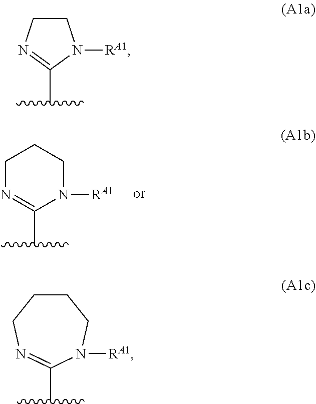

wherein each one of the above-depicted groups A1a, A1b and A1c is optionally substituted with one or more (e.g., one, two or three) groups R^{A2}. It is preferred that d is 1 or 2, i.e. that the group A1 is a group A1a or a group A1b, wherein said group A1a or said group A1b is optionally substituted with one or more groups R^{A2}. More preferably, d is 1, i.e. the group A1 is a group A1a (which is optionally substituted with one or more groups R^{A2}).

For each one of the above-depicted groups A2 to A6, it is preferred that the ring which is marked with the symbol “(N)” contains 0, 1 or 2 nitrogen ring atom(s), more preferably 0 or 1 nitrogen ring atom(s), and even more preferably 0 nitrogen ring atoms, while all remaining ring atoms in the respective ring are carbon atoms. Accordingly, it is particularly preferred that the 6-membered ring marked with the symbol “(N)” in any one of A2 to A6, which forms part of the corresponding fused ring system, is a phenyl ring.

For each of the above-depicted groups A2 and A7, the variable p may be 0, 1, 2 or 3, and the variable q may be 0, 1 or 2, whereby p and q cannot both be 0, i.e. the sum of p and q is equal to or greater than 1 (p+q≥1). Corresponding examples of the group A2 include a group A2 wherein p is 1 and q is 0, a group A2 wherein p is 0 and q is 1, a group A2 wherein p is 1 and q is 1, a group A2 wherein p is 2 and q is 0, a group A2 wherein p is 2 and q is 1, a group A2 wherein p is 0 and q is 2, a group A2 wherein p is 1 and q is 2, or a group A2 wherein p is 2 and q is 2. Corresponding examples of the group A7 include a group A7 wherein p is 1 and q is 0, a group A7 wherein p is 0 and q is 1, a group A7 wherein p is 1 and q is 1, a group A7 wherein p is 2 and q is 0, a group A7 wherein p is 2 and q is 1, a group A7 wherein p is 0 and q is 2, a group A7 wherein p is 1 and q is 2, or a group A7 wherein p is 2 and q is 2. For each of A2 and A7, it is preferred that p is 0, 1 or 2, and that q is 0, 1 or 2, with the proviso that p and q are not both 0 (i.e., p+q≥1). More preferably, p is 0, 1 or 2, and q is 0 or 1, with the proviso that p and q are not both 0. Moreover, the sum of p and q is preferably 1, 2, 3 or 4, more preferably 1, 2 or 3, even more preferably 1 or 2, yet even more preferably 1.

Accordingly, if ring A is a group A2, it is preferred that the group A2 is selected from the following groups A2a, A2b, A2c, A2d and A2e:

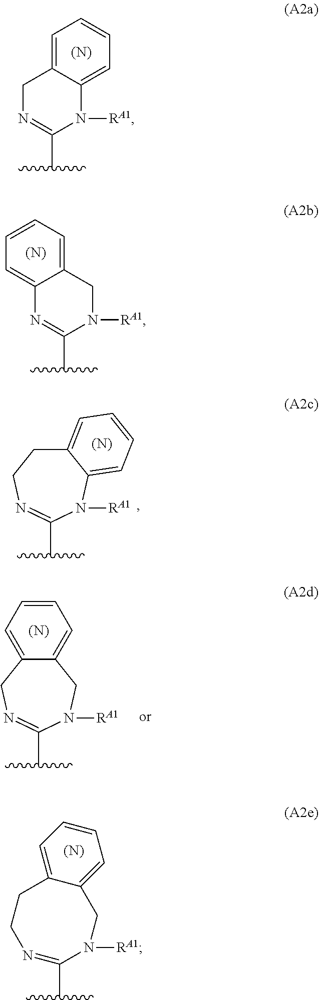

wherein ring A (i.e., each one of the above-depicted groups A2a, A2b, A2c, A2d and A2e) is optionally substituted with one or more groups R^{A2}.

More preferably, the group A2 is selected from the following groups A2a1, A2a2, A2b1, A2c1, A2d1 and A2e1:

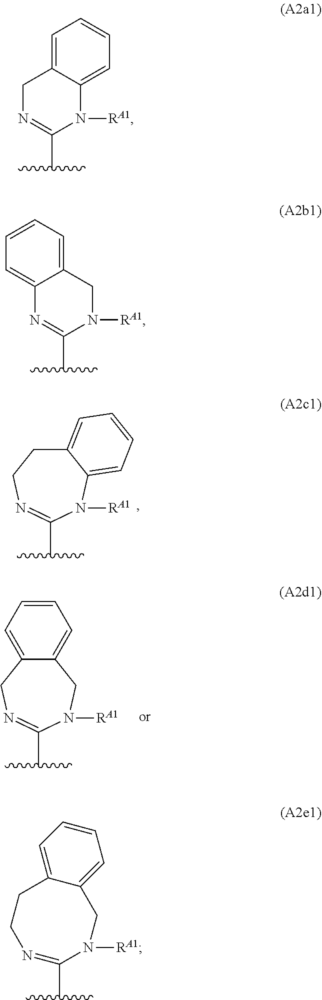

wherein ring A (i.e., each one of the above-depicted groups A2a1, A2a2, A2b1, A2c1, A2d1 and A2e1) is optionally substituted with one or more groups R^{A2}.

Even more preferably, the group A2 is a group A2a1, A2c1 or A2d1:

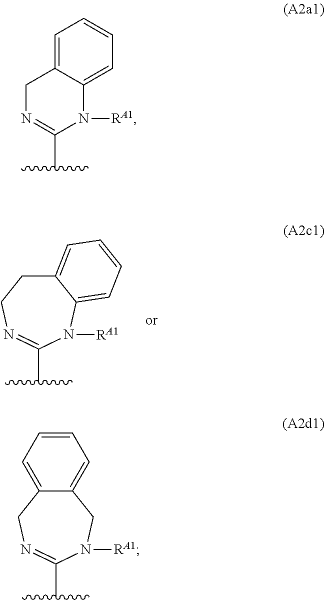

which is optionally substituted with one or more groups R^{A2}.

Yet even more preferably, the group A2 is a group A2a1 or A2c1:

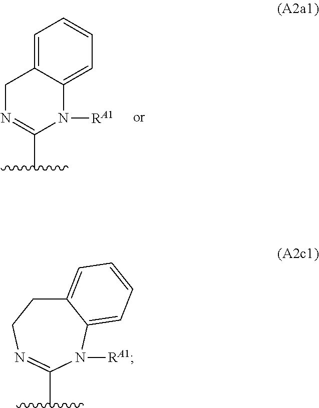

which is optionally substituted with one or more groups R^{A2}.

Still more preferably, the group A2 is a group A2a1 which is optionally substituted with one or more groups R^{A2}.

For each one of the above-depicted groups A7 to A11, the 5-membered ring which is marked with the symbol “X” (and which forms part of the corresponding fused ring system) is aromatic and contains 1, 2 or 3 ring atom(s) which is/are each independently selected from nitrogen, oxygen and sulfur, while the remaining ring atoms are carbon atoms. Preferably, said 5-membered ring marked with the symbol “X” is aromatic and contains 1 or 2 ring atom(s) selected independently from nitrogen, oxygen and sulfur, while the remaining ring atoms are carbon atoms. More preferably, said 5-membered ring marked with the symbol “X” is aromatic, contains 1 nitrogen ring atom, and additionally contains 0 or 1 further ring heteroatom selected from nitrogen, oxygen and sulfur, while the remaining ring atoms are carbon atoms (i.e., said 5-membered ring contains two nitrogen ring atoms, or one nitrogen and one oxygen ring atom, or one nitrogen and one sulfur ring atom, while all remaining ring atoms are carbon atoms).

Corresponding examples of the 5-membered ring marked with the symbol “X” (i.e.,

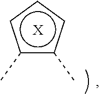

which is comprised in the ring groups A7 to A11, include, in particular, any one of the following groups:

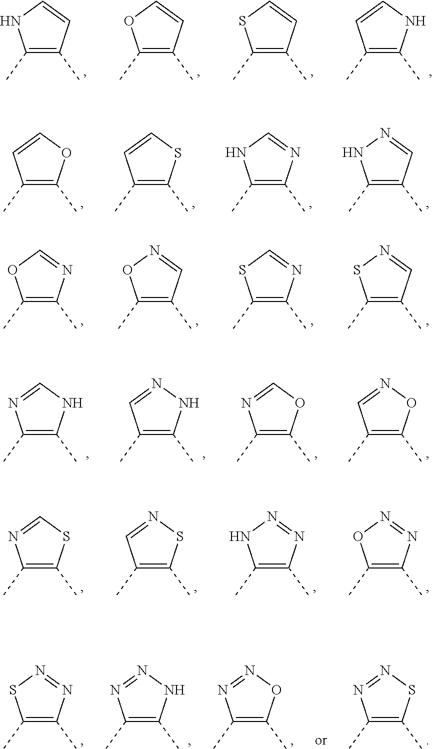

In accordance with the definition of ring A, each one of the groups A7 to A11 is optionally substituted with one or more groups R^{A2}. Thus, each one of the above-depicted exemplary 5-membered ring groups may also be substituted with one or more groups R^{A2}.

It will be understood that the 5-membered ring marked with the symbol “X” is aromatic and that, in the case of the ring groups A8, A9, A10 and A11, the double bonds in this 5-membered ring are conjugated with the double bonds in the other (fused) ring comprised in the same fused ring system. Thus, for example, if the 5-membered ring marked with the symbol “X” comprised in the ring group A8, A9, A10 or A11 is a group

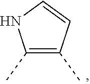

then the resulting ring group A8, A9, A10 or A11 will have the following structure:

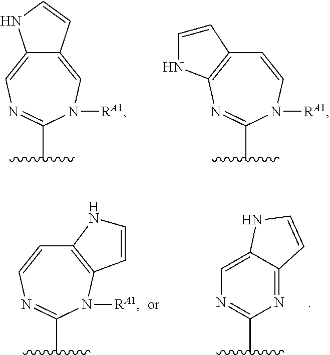

Moreover, in the case of the ring group A7, further examples of the 5-membered ring marked with the symbol “X” (i.e.,

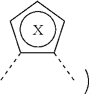

include any one of the following groups:

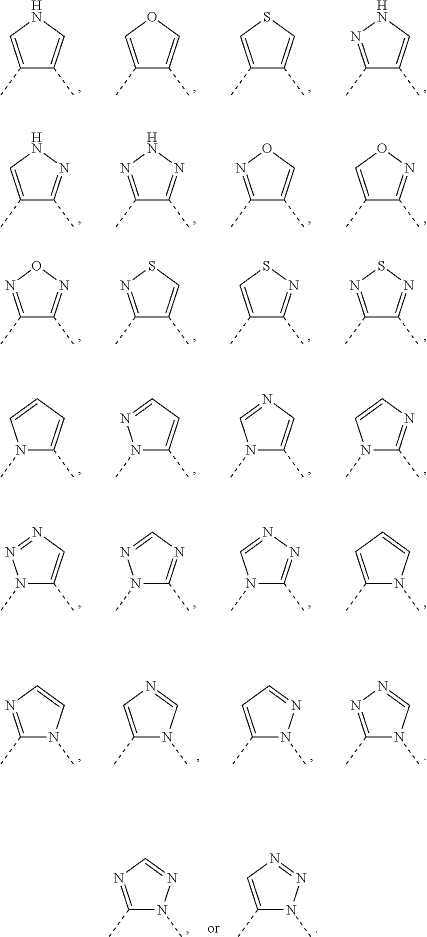

As explained above, the ring group A7 is optionally substituted with one or more groups R^{A2}. Accordingly, each one of the above-depicted exemplary 5-membered ring groups may also be substituted with one or more groups R^{A2}.

In accordance with the above, it is preferred that ring A is a group selected from A1, A2, A3, A4, A5, A7, A8, A9 and A10. More preferably, ring A is a group selected from A1, A2, A3, A4 and A5. Even more preferably, ring A is a group A1 or A2. The group A1 is preferably a group A1a or A1b, more preferably a group A1a. The group A2 is preferably a group selected from A2a, A2b, A2c, A2d and A2e, more preferably a group selected from A2a1, A2a2, A2b1, A2c1, A2d1 and A2e1, even more preferably a group A2a1, A2c1 or A2d1, yet even more preferably a group A2a1 or A2c1, still more preferably a group A2a1. It will be understood that each of the groups mentioned in this paragraph is optionally substituted with one or more groups R^{A2}.

Ring A in formula (I) may also be any one of the specific ring A groups comprised in any one of the compounds described in the examples section, particularly any one of Examples 1 to 200.

n is 0, 1 or 2. Preferably, n is 0.

It will be understood that the variable n indicates the number of ═O groups attached to the sulfur atom in the corresponding group —S(═O)ₙ— in the compound of formula (I). Thus, if n is 0, then the group —S(═O)ₙ— is a group —S—. If n is 1, then the group —S(═O)ₙ— is a group —SO—. If n is 2, then the group —S(═O)ₙ— is a group —SO₂—. It is preferred that n is 0, i.e. that the group —S(═O)ₙ— is a group —S—.

Specific preferred examples of the moiety —S(═O)ₙ-(ring A) include, in particular,

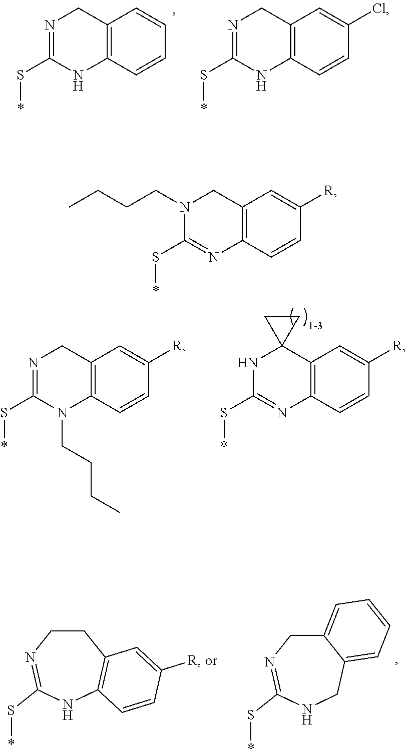

wherein each R is independently hydrogen or halogen (preferably —Cl). Further preferred examples of the moiety —S(═O)ₙ-(ring A) include, e.g.,

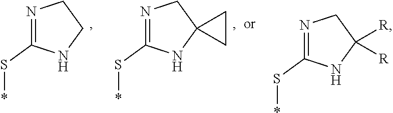

wherein each R is independently C₁₋₅ alkyl, —(C₀₋₅ alkylene)-aryl, or —(C₀₋₅ alkylene)-heteroaryl).

L is a covalent bond or C₁₋₅ alkylene, wherein said alkylene is optionally substituted with one or more (e.g., one, two or three) groups R^{L}, wherein one or more (e.g., one, two or three) —CH₂— units comprised in said alkylene are each optionally replaced by a group independently selected from —O—, —NH—, —N(C₁₋₅ alkyl)-, —CO—, —S—, —SO—, —SO₂—, carbocyclylene, and heterocyclylene, and wherein each R^{L} is independently selected from —OH, —O(C₁₋₅ alkyl), —SH, —S(C₁₋₅ alkyl), —NH₂, —NH(C₁₋₅ alkyl), —N(C₁₋₅ alkyl)(C₁₋₅ alkyl), halogen, —CF₃, —CN, C₁₋₅ alkyl, cycloalkyl, and heterocycloalkyl.

If one or more —CH₂— units comprised in said C₁₋₅ alkylene (as group L) are each optionally replaced by a group as defined above, it is preferred that said group is independently selected from —O—, —NH—, —N(C₁₋₅ alkyl)-, —CO—, carbocyclylene (e.g., cycloalkylene or arylene), and heterocyclylene (e.g., heterocycloalkylene or heteroarylene), more preferably from —CO—, cycloalkylene, arylene, heterocycloalkylene, and heteroarylene. In particular, the terminal —CH₂— unit comprised in said C₁₋₅ alkylene, which is attached to ring B, may be replaced by a group —CO—.

It is preferred that said cycloalkylene (which may replace a —CH₂— unit in the C₁₋₅ alkylene as group L) is a C₃₋₅ cycloalkylene, more preferably a cyclopropylene. Furthermore, in a preferred embodiment, said cycloalkylene (including said C₃₋₅ cycloalkylene or said cyclopropylene) is attached via the same ring carbon atom to the remainder of the compound (i.e., that said cycloalkylene is a cycloalkan-1,1-diyl group). In a further preferred embodiment, said cycloalkylene (including said C₃₋₅ cycloalkylene or said cyclopropylene) is attached via distinct ring carbon atoms to the remainder of the compound (e.g., via directly adjacent ring carbon atoms, or via those ring carbon atoms that have the greatest distance in terms of connecting ring atoms); thus, said cycloalkylene may be, e.g., cyclopropan-1,2-diyl, cyclobutan-1,2-diyl, cyclobutan-1,3-diyl, cyclopentan-1,2-diyl, cyclopentan-1,3-diyl, cyclohexan-1,2-diyl, cyclohexan-1,3-diyl, or cyclohexan-1,4-diyl. Moreover, it is preferred that said heterocycloalkylene (which may replace a —CH₂— unit in the C₁₋₅ alkylene as group L) is a heterocycloalkylene having 3 to 5 ring members, more preferably a heterocycloalkylene having 3 to 5 ring members wherein 1 ring member is a heteroatom selected from O, S and N (and the remaining ring members are carbon atoms), such as, e.g., oxetanylene. In a preferred embodiment, said heterocycloalkylene is attached via the same ring carbon atom to the remainder of the compound (as in, e.g., oxetan-3,3-diyl). In a further preferred embodiment, said heterocycloalkylene is attached via distinct ring atoms to the remainder of the compound (e.g., via directly adjacent ring atoms, or via those ring atoms that have the greatest distance in terms of connecting ring atoms). It is preferred that said arylene (which may replace a —CH₂— unit in the C₁₋₅ alkylene as group L) is phenylene, e.g., phen-1,2-diyl, phen-1,3-diyl, or phen-1,4-diyl; the group L may thus be, e.g., phen-1,3-diyl, phen-1,4-diyl, —CH₂-phen-1,3-diyl, —CH₂-phen-1,4-diyl, -phen-1,3-diyl-CH₂—, or -phen-1,4-diyl-CH₂—. Furthermore, it is preferred that said heteroarylene (which may replace a —CH₂— unit in the C₁₋₅ alkylene as group L) is a monocyclic heteroarylene, e.g., pyridinylene (e.g., pyridin-2,4-diyl, pyridin-2,5-diyl, pyridin-2,6-diyl, or pyridin-3,5-diyl) or imidazolylene (e.g., imidazol-2,4-diyl); the group L may thus be, e.g., pyridin-2,4-diyl, pyridin-2,5-diyl, pyridin-2,6-diyl, pyridin-3,5-diyl, —CH₂-pyridin-2,4-diyl, —CH₂-pyridin-2,5-diyl, —CH₂-pyridin-2,6-diyl, —CH₂-pyridin-3,5-diyl, pyridin-2,4-diyl-CH₂—, pyridin-2,5-diyl-CH₂—, pyridin-2,6-diyl-CH₂—, pyridin-3,5-diyl-CH₂—, imidazol-2,4-diyl, —CH₂-imidazol-2,4-diyl, or imidazol-2,4-diyl-CH₂—. It will be understood that if L is methylene in which one —CH₂— unit is replaced, e.g., by cyclopropan-1,1-diyl, then the resulting group L is cyclopropan-1,1-diyl.

It is furthermore preferred that each R^{L} is independently selected from —OH, —O(C₁₋₅ alkyl) and C₁₋₅ alkyl. In particular, each R^{L} may be independently selected from —OH and —O(C₁₋₅ alkyl).

In accordance with the above, it is preferred that L is a covalent bond or C₁₋₅ alkylene (e.g., C₁₋₃ alkylene, such as —CH₂—, —CH₂CH₂— or —CH₂CH₂CH₂—), wherein said C₁₋₅ alkylene is optionally substituted with one or more (e.g., one or two) groups R^{L}, and further wherein one —CH₂— unit comprised in said C₁₋₅ alkylene is optionally replaced by —CO—, carbocyclylene (e.g., cycloalkylene) or heterocyclylene (e.g., heterocycloalkylene). Preferred examples of L include, in particular, a linear C₃₋₅ alkylene (e.g., —CH₂CH₂CH₂—, —CH₂CH₂CH₂CH₂—, or —CH₂CH₂CH₂CH₂CH₂—) which is optionally substituted with one or more groups R^{L}. More preferably, L is a covalent bond, —CH₂—, —CH₂CH₂—, —CH₂CH₂CH₂—, —CH₂CH₂CH₂CH₂—, —CH₂—C(—CH₃)(—CH₃)—, —C(—CH₃)(—CH₃)—CH₂—, —(CH₂)₃—C(—CH₃)(—CH₃)—, —(CH₂)₃—CH(—CH₂CH₃)—, —C(—CH₃)(—CH₃)—C(—CH₃)(—CH₃)—, —(CH₂)₃—CH(—CH(—CH₃)—CH₃)—, —CH₂C(═O)—, cycloalkylene (e.g., cyclopropan-1,1-diyl), arylene, heterocycloalkylene (e.g., oxetan-3,3-diyl), or heteroarylene, wherein said —CH₂C(═O)— is attached via its C(═O) carbon atom to ring B and via its CH₂ carbon atom to the group —S(═O)ₙ-in formula (I). Even more preferably, L is a covalent bond, —CH₂—, —CH₂CH₂—, —CH₂CH₂CH₂—, —CH₂CH₂CH₂CH₂—, —CH₂—C(—CH₃)(—CH₃)—, or —C(—CH₃)(—CH₃)—CH₂—.

If ring A is a group A1, then ring B is selected from any one of the following groups:

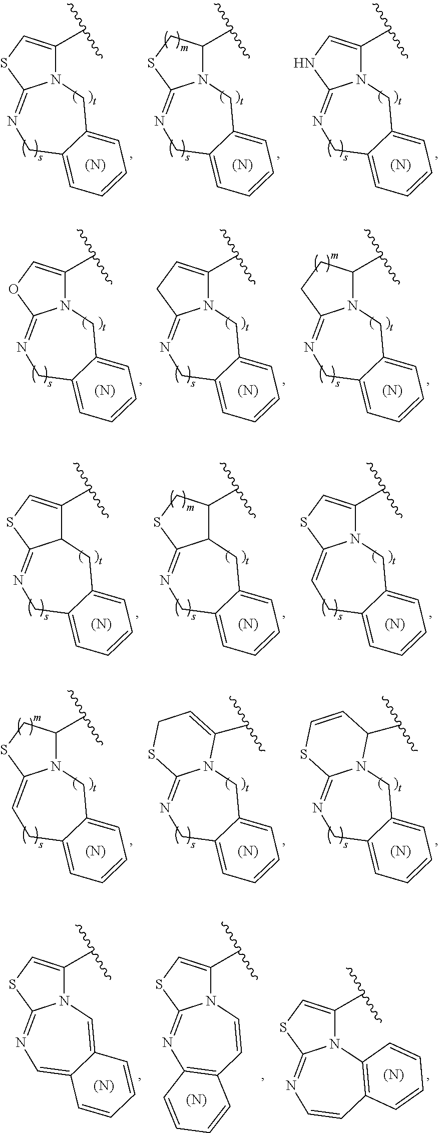

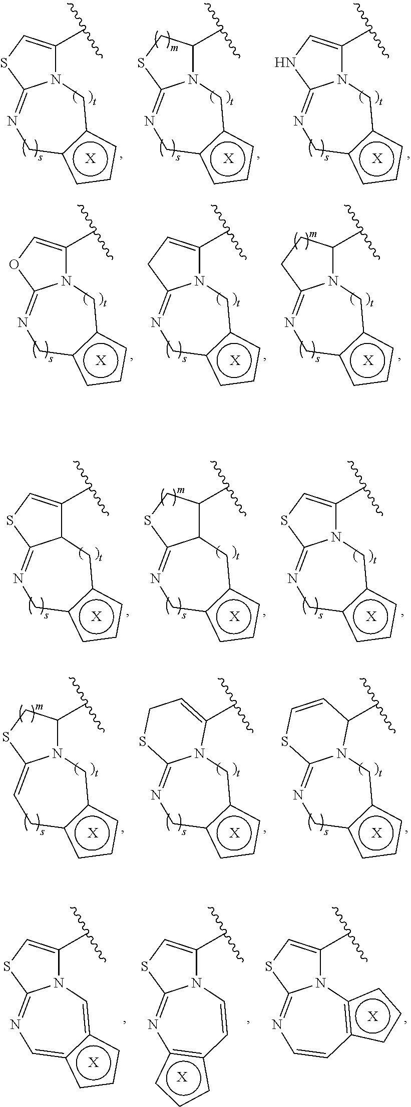

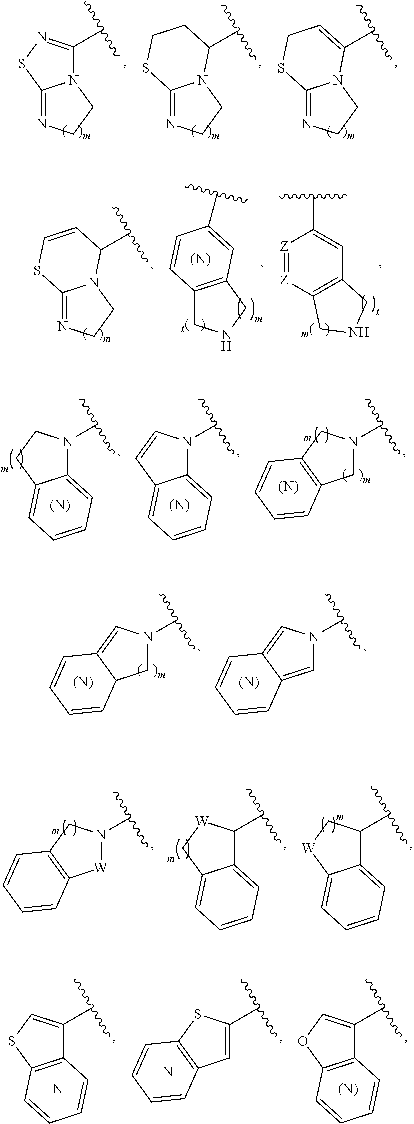

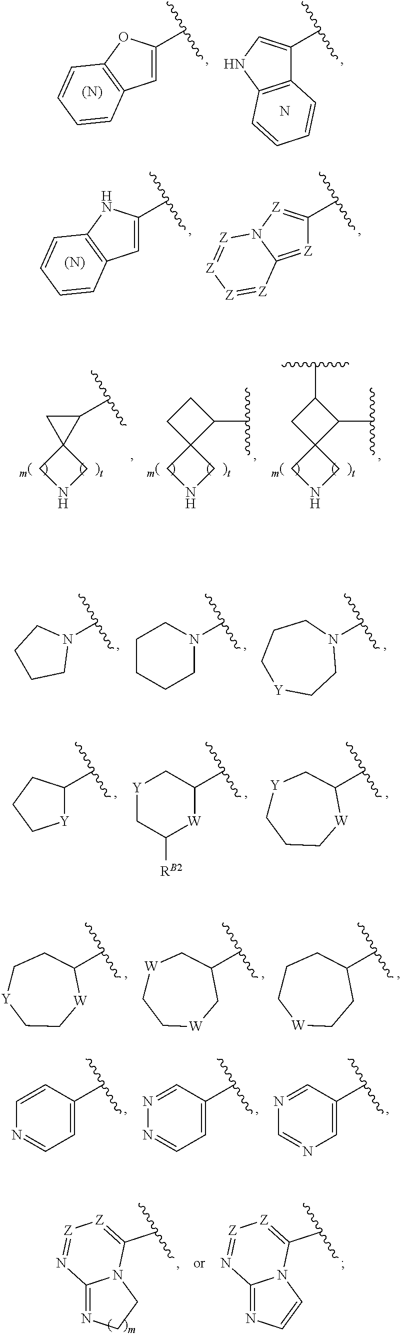

wherein each of the above-depicted groups is optionally substituted with one or more (e.g., one, two or three) groups R^{B1};
wherein each s is independently 0, 1 or 2 (preferably, each s is independently 0 or 1);
wherein each t is independently 0, 1, 2 or 3 (preferably, each t is independently 0, 1 or 2, more preferably 0 or 1);
wherein each m is independently 1, 2 or 3 (preferably, each m is independently 1 or 2);
wherein each ring atom W is independently selected from S, O, SO₂ and NH;
wherein each ring atom Y is independently selected from S, O, SO₂, NH and CH₂;
wherein each ring atom Z is independently C or N;
wherein the symbol “(N)” depicted inside a ring indicates that 0, 1, 2 or 3 ring atom(s) of the respective ring is/are nitrogen ring atom(s) (preferably, the symbol “(N)” depicted inside a ring indicates that 0, 1 or 2 ring atom(s), more preferably 0 or 1 ring atom(s), of the respective ring is/are nitrogen ring atom(s));
wherein the symbol “N” depicted inside a ring indicates that 1, 2 or 3 ring atom(s) of the respective ring is/are nitrogen ring atom(s) (preferably, the symbol “N” depicted inside a ring indicates that 1 or 2 ring atom(s), more preferably 1 ring atom, of the respective ring is/are nitrogen ring atom(s)); and
wherein the symbol “X” depicted inside a 5-membered ring indicates that the corresponding ring is aromatic and that 1, 2 or 3 ring atom(s) of said ring is/are each independently selected from nitrogen, oxygen and sulfur, while the remaining ring atoms are carbon atoms (e.g., said 5-membered ring may be any one of the exemplary or preferred rings described herein above in connection with the 5-membered rings marked with the symbol “X” comprised in the ring groups A7 or A8 to A11).

It will be understood that the variables s, t and m indicate the number of the respective ring atoms. If s is 0, the corresponding ring atom is absent, i.e. is replaced by a covalent bond. Likewise, if t is 0, the corresponding ring atom is absent, i.e. is replaced by a covalent bond. If, for example, m in the group

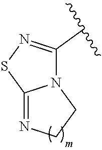

is 1, 2 or 3, then the corresponding group will have the following structure:

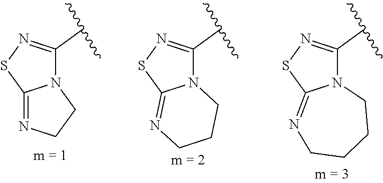 [SMILES: *C1=NSC2=NCCN12 ; *C1=NSC2=NCCCN12 ; *C1=NSC2=NCCCCN12]

As explained above, the symbol “(N)” inside a ring, as in

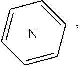

indicates that 0, 1, 2 or 3 ring atom(s) of the respective ring is/are nitrogen ring atom(s). The remaining ring atoms are carbon ring atoms. Thus, a ring

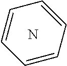

(which may form part of a ring system) may be a phenyl ring, a pyridine ring, a diazine ring, or a triazine ring. Where the symbol “(N)” is depicted inside a ring, it is preferred that 0, 1 or 2 ring atom(s) of the respective ring is/are nitrogen ring atom(s); more preferably, 0 or 1 ring atom(s) of the respective ring is/are nitrogen ring atom(s).

Moreover, as also explained above, the symbol “N” inside a ring, as in

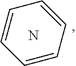

indicates that 1, 2 or 3 ring atom(s) of the respective ring is/are nitrogen ring atom(s). The remaining ring atoms are carbon ring atoms. Thus, a ring

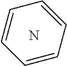

(which may form part of a ring system) may be a pyridine ring, a diazine ring, or a triazine ring. Where the symbol “N” is depicted inside a ring, it is preferred that 1 or 2 ring atom(s) of the respective ring is/are nitrogen ring atom(s); more preferably, 1 ring atom of the respective ring is a nitrogen ring atom.

Preferably, if ring A is a group A1, then ring B is selected from any one of the following groups:

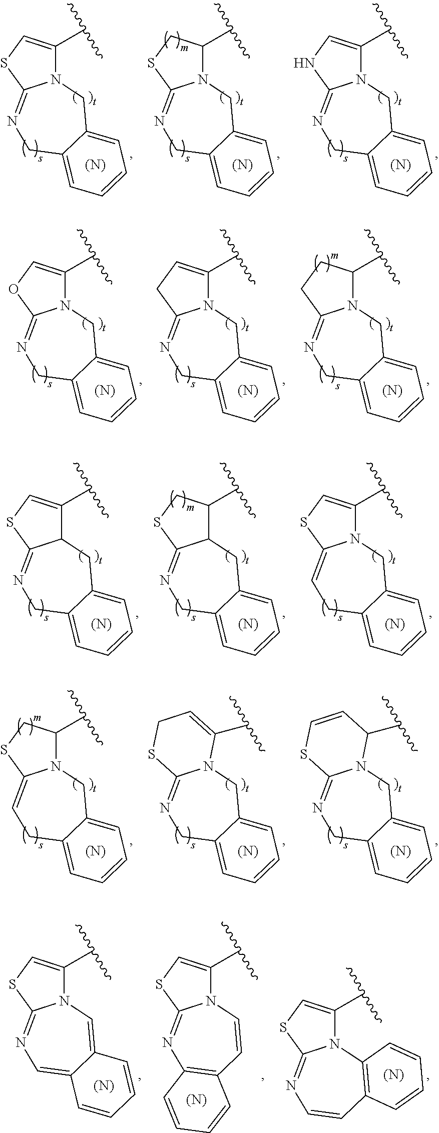

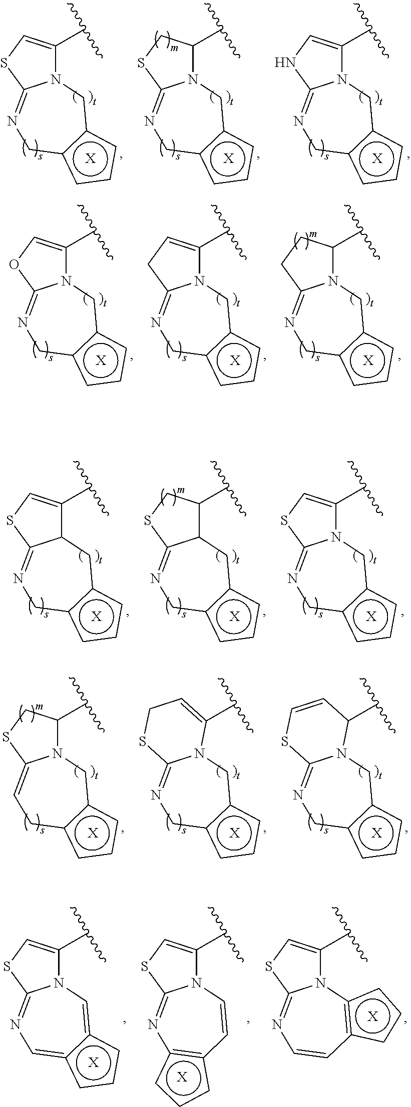

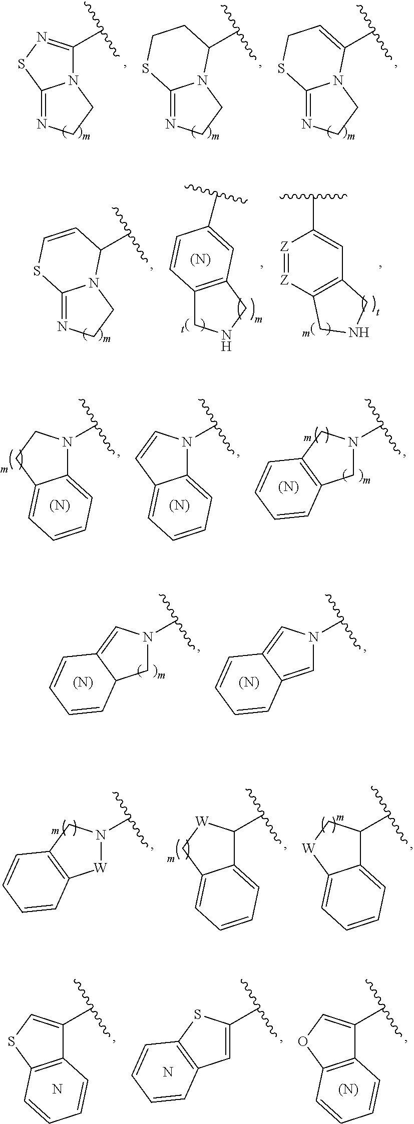

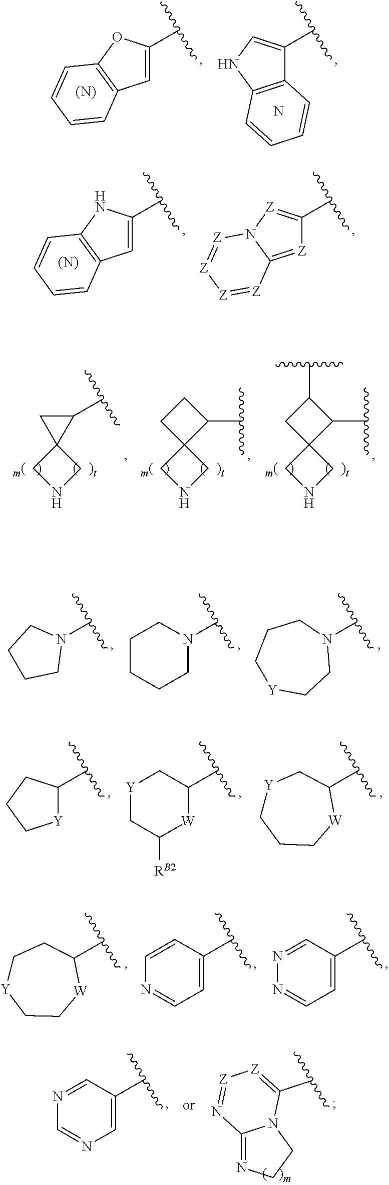

wherein each of the above-depicted groups is optionally substituted with one or more (e.g., one, two or three) groups R^{B1};
wherein each s is independently 0, 1 or 2 (preferably, each s is independently 0 or 1);
wherein each t is independently 0, 1, 2 or 3 (preferably, each t is independently 0, 1 or 2, more preferably 0 or 1);
wherein each m is independently 1, 2 or 3 (preferably, each m is independently 1 or 2);
wherein each ring atom W is independently selected from S, O, SO₂ and NH;
wherein each ring atom Y is independently selected from S, O, SO₂, NH and CH₂;
wherein each ring atom Z is independently C or N;
wherein the symbol “(N)” depicted inside a ring indicates that 0, 1, 2 or 3 ring atom(s) of the respective ring is/are nitrogen ring atom(s) (preferably, the symbol “(N)” depicted inside a ring indicates that 0, 1 or 2 ring atom(s), more preferably 0 or 1 ring atom(s), of the respective ring is/are nitrogen ring atom(s));
wherein the symbol “N” depicted inside a ring indicates that 1, 2 or 3 ring atom(s) of the respective ring is/are nitrogen ring atom(s) (preferably, the symbol “N” depicted inside a ring indicates that 1 or 2 ring atom(s), more preferably 1 ring atom, of the respective ring is/are nitrogen ring atom(s)); and
wherein the symbol “X” depicted inside a 5-membered ring indicates that the corresponding ring is aromatic and that 1, 2 or 3 ring atom(s) of said ring is/are each independently selected from nitrogen, oxygen and sulfur, while the remaining ring atoms are carbon atoms (e.g., said 5-membered ring may be any one of the exemplary or preferred rings described herein above in connection with the 5-membered rings marked with the symbol “X” comprised in the ring groups A7 or A8 to A11).

More preferably, if ring A is a group A1, then ring B is selected from any one of the following groups:

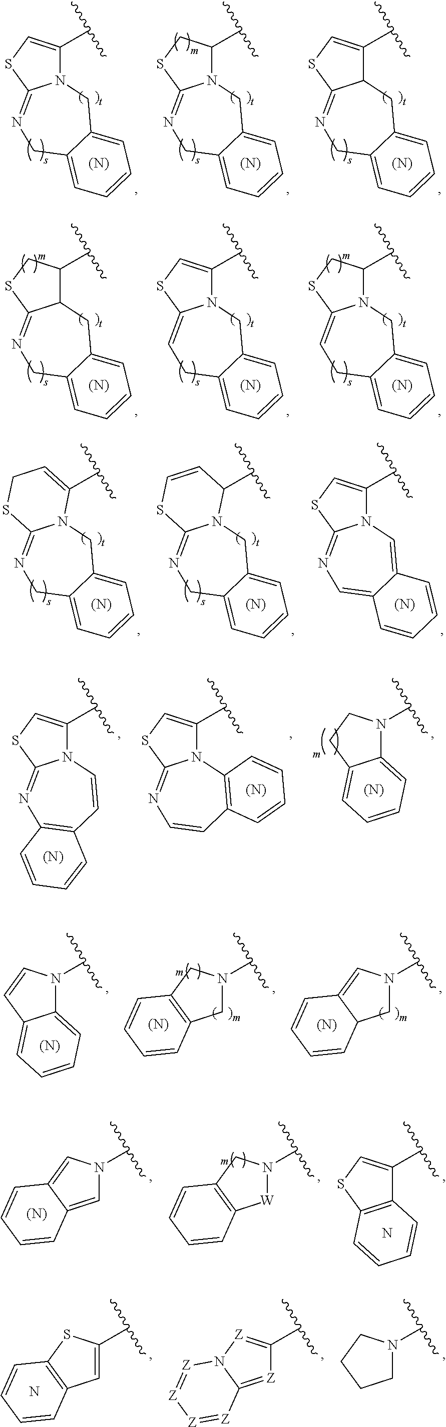

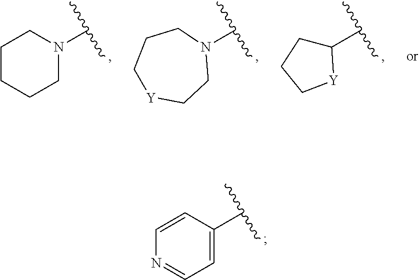

wherein each of the above-depicted groups is optionally substituted with one or more (e.g., one, two or three) groups R^{B1};
wherein each s is independently 0, 1 or 2 (preferably, each s is independently 0 or 1);
wherein each t is independently 0, 1, 2 or 3 (preferably, each t is independently 0, 1 or 2, more preferably 0 or 1);
wherein each m is independently 1, 2 or 3 (preferably, each m is independently 1 or 2); and
wherein each ring atom W is independently selected from S, O, SO₂ and NH;
wherein each ring atom Y is independently selected from S, O, SO₂, NH and CH₂;
wherein each ring atom Z is independently C or N;
wherein the symbol “(N)” depicted inside a ring indicates that 0, 1, 2 or 3 ring atom(s) of the respective ring is/are nitrogen ring atom(s) (preferably, the symbol “(N)” depicted inside a ring indicates that 0, 1 or 2 ring atom(s), more preferably 0 or 1 ring atom(s), of the respective ring is/are nitrogen ring atom(s)); and
wherein the symbol “N” depicted inside a ring indicates that 1, 2 or 3 ring atom(s) of the respective ring is/are nitrogen ring atom(s) (preferably, the symbol “N” depicted inside a ring indicates that 1 or 2 ring atom(s), more preferably 1 ring atom, of the respective ring is/are nitrogen ring atom(s)).

Even more preferably, if ring A is a group A1, then ring B is selected from any one of the following groups:

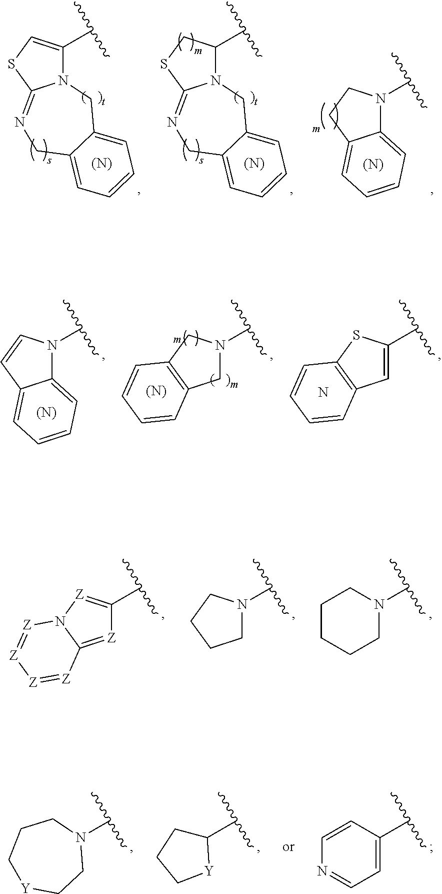

wherein each of the above-depicted groups is optionally substituted with one or more (e.g., one, two or three) groups R^{B1};
wherein each s is independently 0, 1 or 2 (preferably, each s is independently 0 or 1);
wherein each t is independently 0, 1, 2 or 3 (preferably, each t is independently 0, 1 or 2, more preferably 0 or 1);
wherein each m is independently 1, 2 or 3 (preferably, each m is independently 1 or 2); and
wherein each ring atom Y is independently selected from S, O, SO₂, NH and CH₂;
wherein each ring atom Z is independently C or N;
wherein the symbol “(N)” depicted inside a ring indicates that 0, 1, 2 or 3 ring atom(s) of the respective ring is/are nitrogen ring atom(s) (preferably, the symbol “(N)” depicted inside a ring indicates that 0, 1 or 2 ring atom(s), more preferably 0 or 1 ring atom(s), of the respective ring is/are nitrogen ring atom(s)); and
wherein the symbol “N” depicted inside a ring indicates that 1, 2 or 3 ring atom(s) of the respective ring is/are nitrogen ring atom(s) (preferably, the symbol “N” depicted inside a ring indicates that 1 or 2 ring atom(s), more preferably 1 ring atom, of the respective ring is/are nitrogen ring atom(s)).

Even more preferably, if ring A is a group A1 then ring B is selected from any one of the following groups:

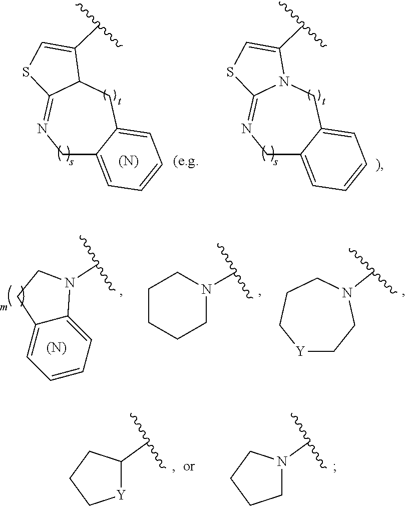

wherein each of the above-depicted groups is optionally substituted with one or more (e.g., one, two or three) groups R^{B1};
wherein each s is independently 0, 1 or 2 (preferably, each s is independently 0 or 1);
wherein each t is independently 0, 1, 2 or 3 (preferably, each t is independently 0, 1 or 2, more preferably 0 or 1); and
wherein each m is independently 1, 2 or 3 (preferably, each m is independently 1 or 2, more preferably 1); and
wherein each ring atom Y is independently selected from NH and CH₂;
wherein the symbol “(N)” depicted inside a ring indicates that 0, 1, 2 or 3 ring atom(s) of the respective ring is/are nitrogen ring atom(s) (preferably, the symbol “(N)” depicted inside a ring indicates that 0, 1 or 2 ring atom(s), more preferably 0 or 1 ring atom(s), even more preferably 0 ring atoms, of the respective ring is/are nitrogen ring atom(s)).

Thus, for example, if ring A is a group A1, then ring B may be selected from any one of the following groups:

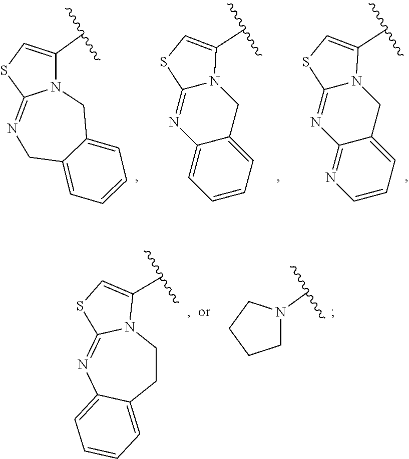 [SMILES: *C1=CSC2=NCc3ccccc3CN12 ; *C1=CSC2=Nc3ccccc3CN12 ; *C1=CSC2=Nc3ncccc3CN12 ; *C1=CSC2=Nc3ccccc3CCN12 ; *N1CCCC1]

wherein each one of the above-depicted groups is optionally substituted with one or more (e.g., one, two or three) groups R^{B1}.

Moreover, if ring A is a group A1, then ring B in formula (I) may also be any one of the specific ring B groups comprised in any one of those compounds of Examples 1 to 200 that have a group A1 as ring A.

If ring A is a group A2, A3, A4, A5, A7, A8, A9 or A10, then ring B is selected from any one of the following groups:

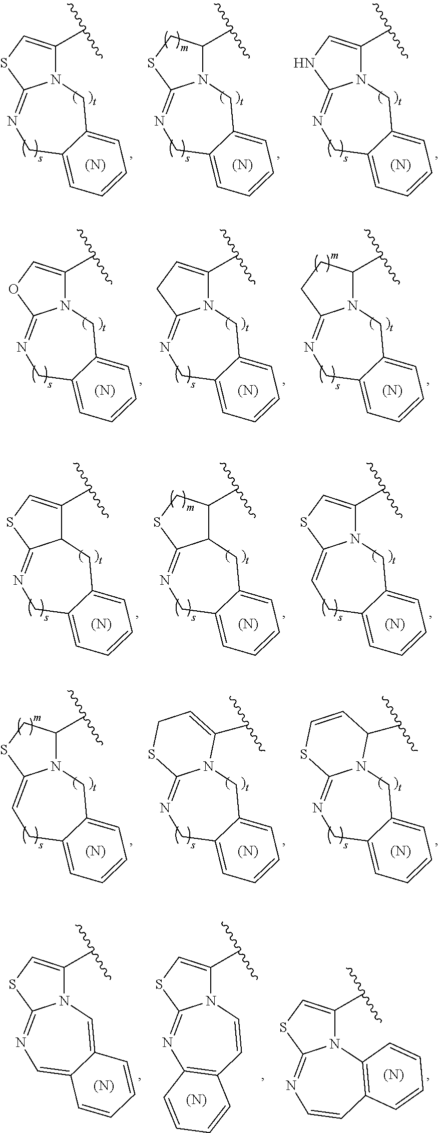

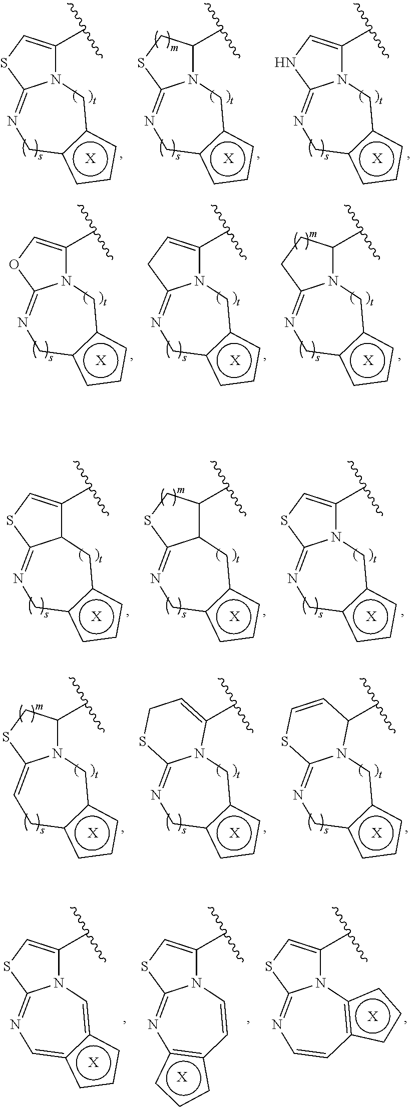

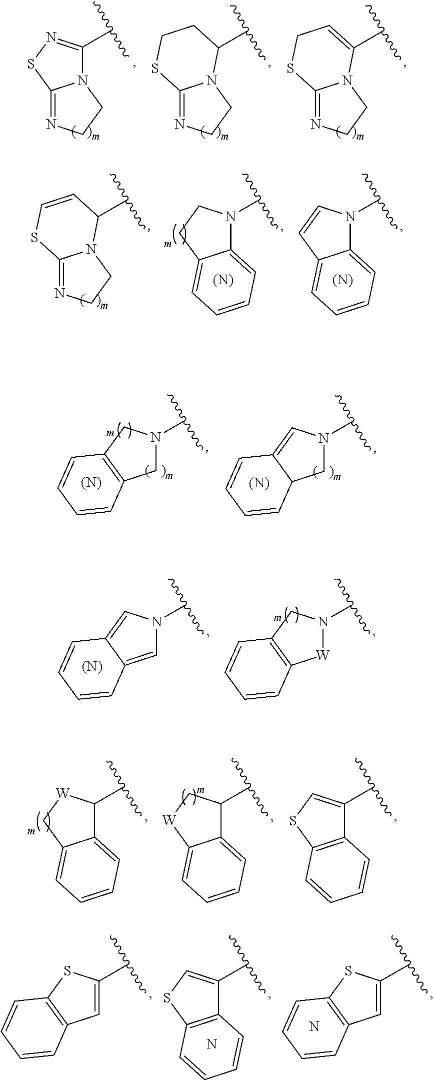

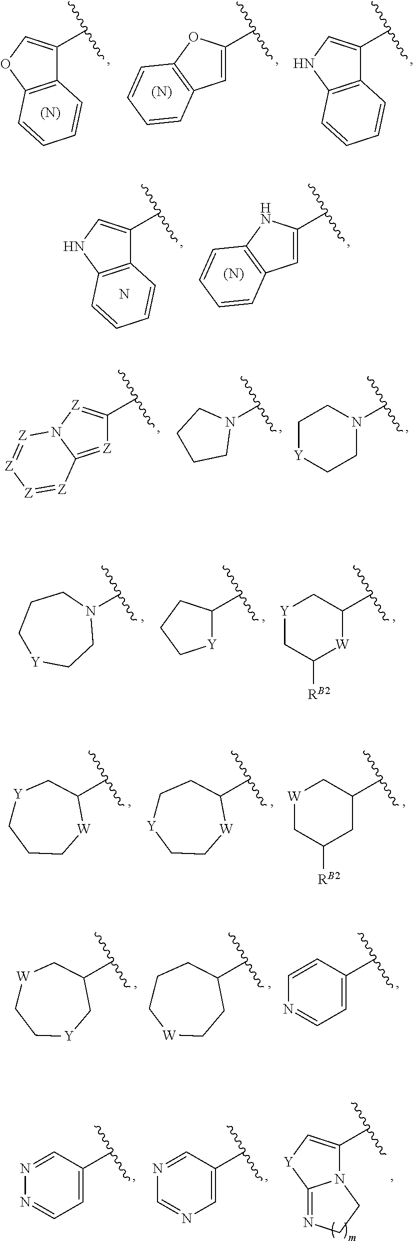

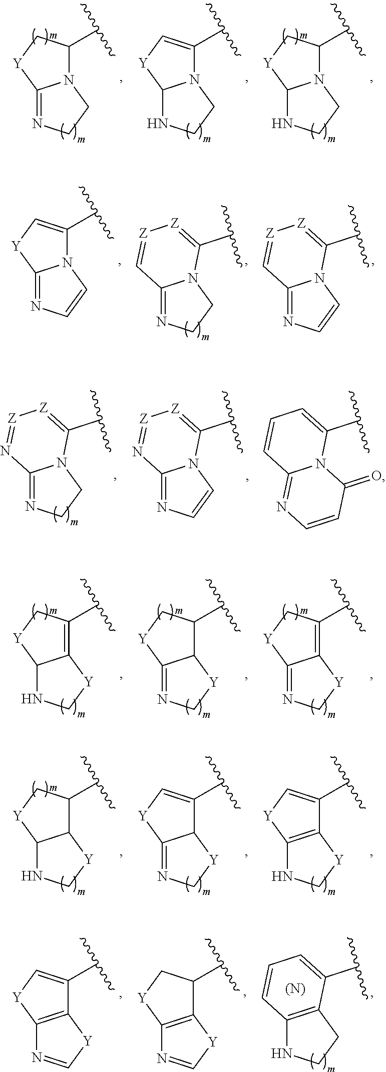

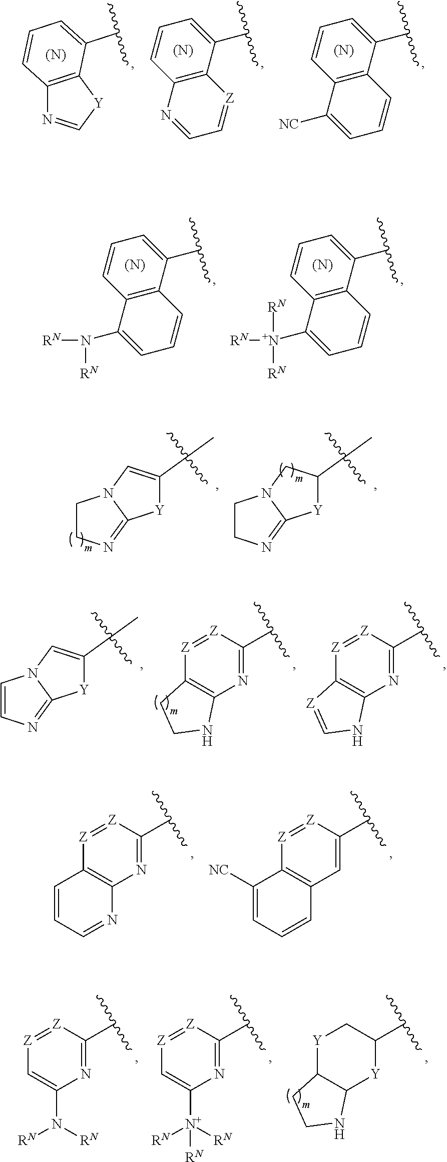

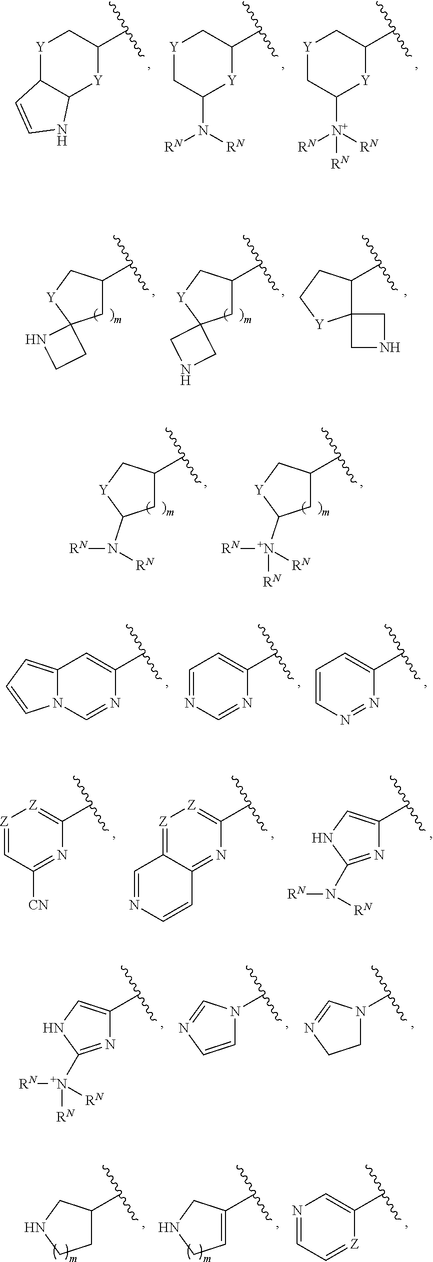

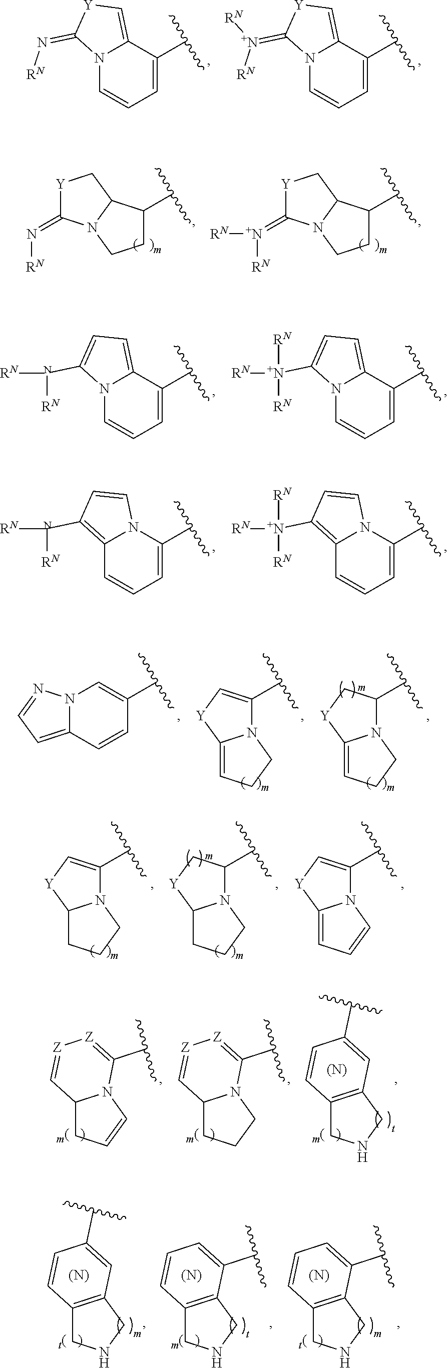

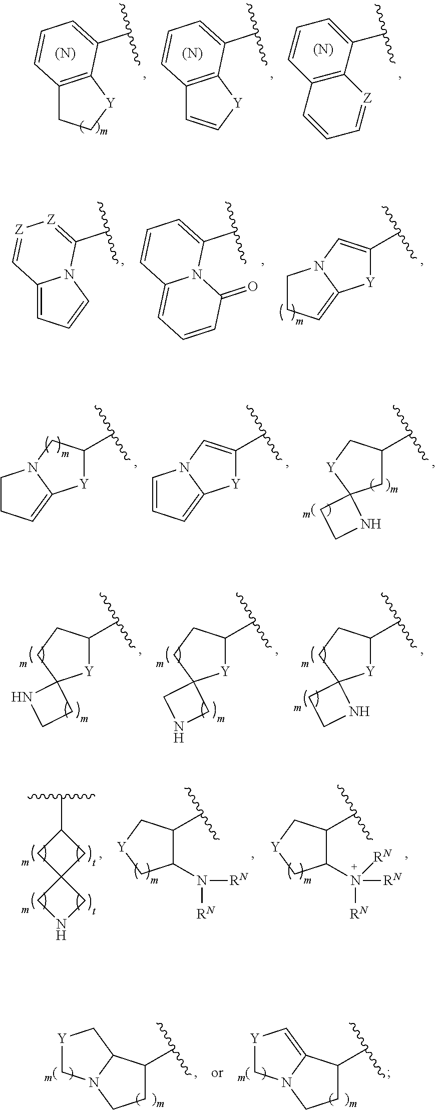

wherein each of the above-depicted groups is optionally substituted with one or more (e.g., one, two or three) groups R^{B1};
wherein each s is independently 0, 1 or 2 (preferably, each s is independently 0 or 1);
wherein each t is independently 0, 1, 2 or 3 (preferably, each t is independently 0, 1 or 2, more preferably 0 or 1);
wherein each m is independently 1, 2 or 3 (preferably, each m is independently 1 or 2);
wherein each ring atom W is independently selected from S, O, SO₂ and NH;
wherein each ring atom Y is independently selected from S, O, SO₂, NH and CH₂;
wherein each ring atom Z is independently C or N;
wherein the symbol “(N)” depicted inside a ring indicates that 0, 1, 2 or 3 ring atom(s) of the respective ring is/are nitrogen ring atom(s) (preferably, the symbol “(N)” depicted inside a ring indicates that 0, 1 or 2 ring atom(s), more preferably 0 or 1 ring atom(s), of the respective ring is/are nitrogen ring atom(s));
wherein the symbol “N” depicted inside a ring indicates that 1, 2 or 3 ring atom(s) of the respective ring is/are nitrogen ring atom(s) (preferably, the symbol “N” depicted inside a ring indicates that 1 or 2 ring atom(s), more preferably 1 ring atom, of the respective ring is/are nitrogen ring atom(s)); and
wherein the symbol “X” depicted inside a 5-membered ring indicates that the corresponding ring is aromatic and that 1, 2 or 3 ring atom(s) of said ring is/are each independently selected from nitrogen, oxygen and sulfur, while the remaining ring atoms are carbon atoms (e.g., said 5-membered ring may be any one of the exemplary or preferred rings described herein above in connection with the 5-membered rings marked with the symbol “X” comprised in the ring groups A7 or A8 to A11).

Preferably, if ring A is a group A2, A3, A4, A5, A7, A8, A9 or A10, then ring B is selected from any one of the following groups:

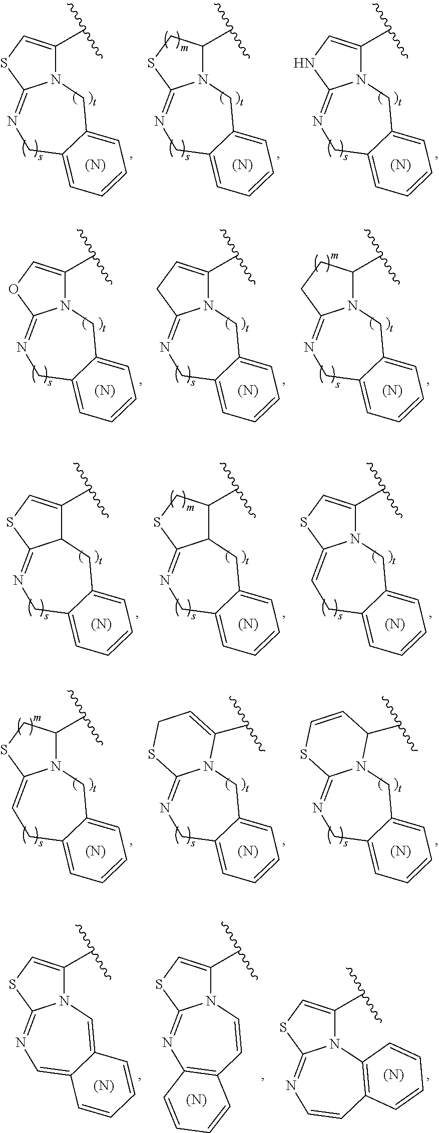

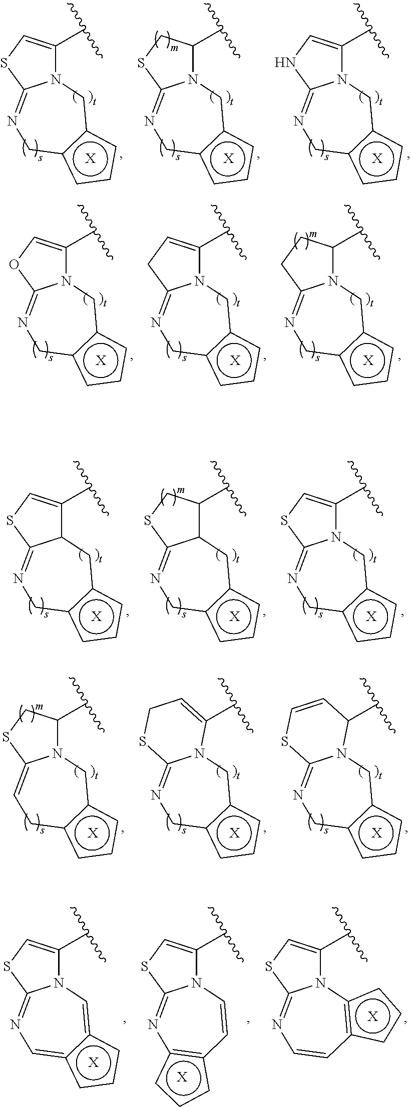

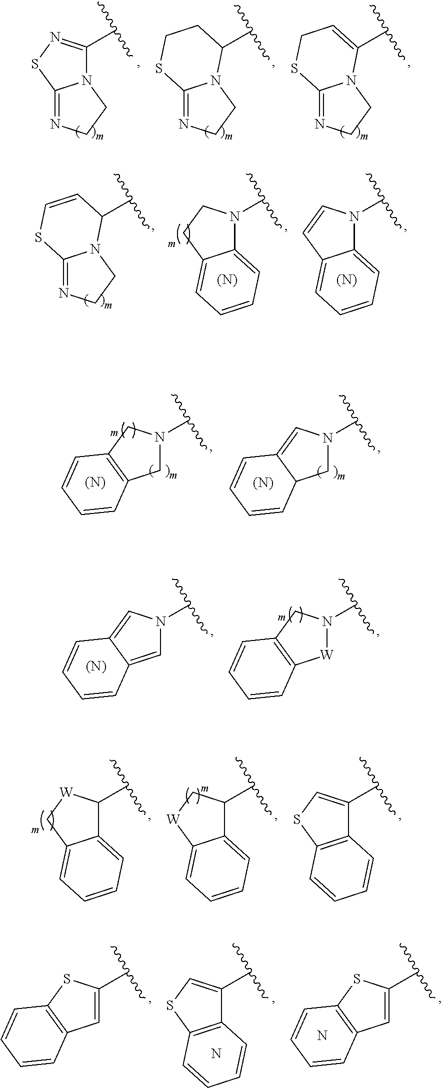

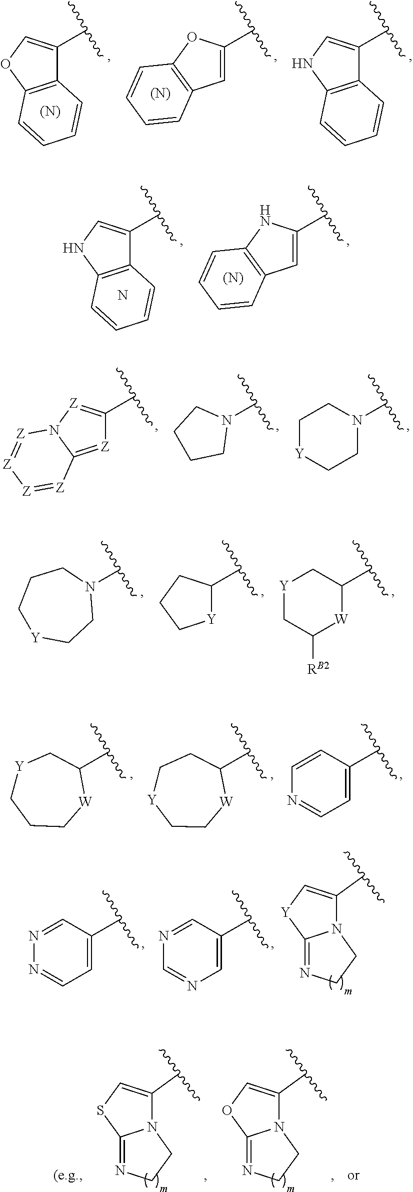

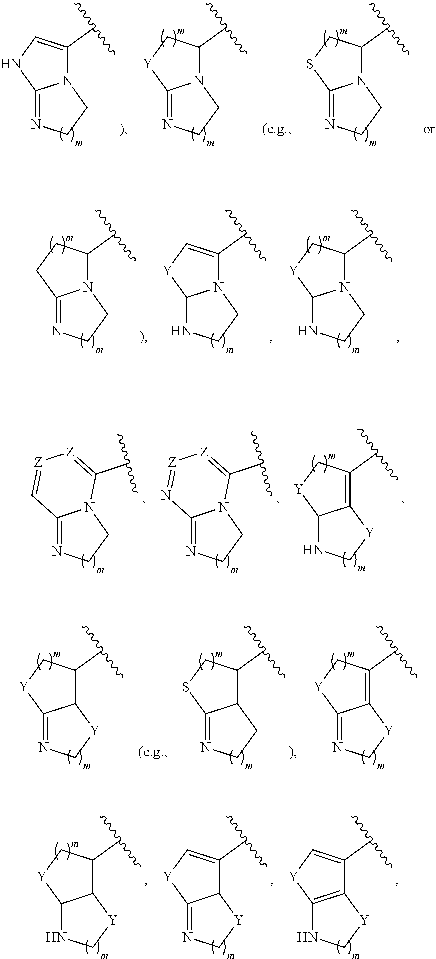

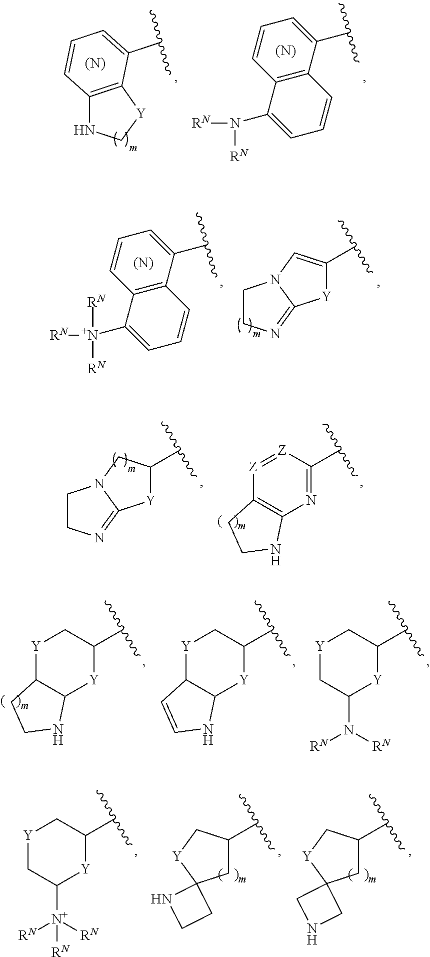

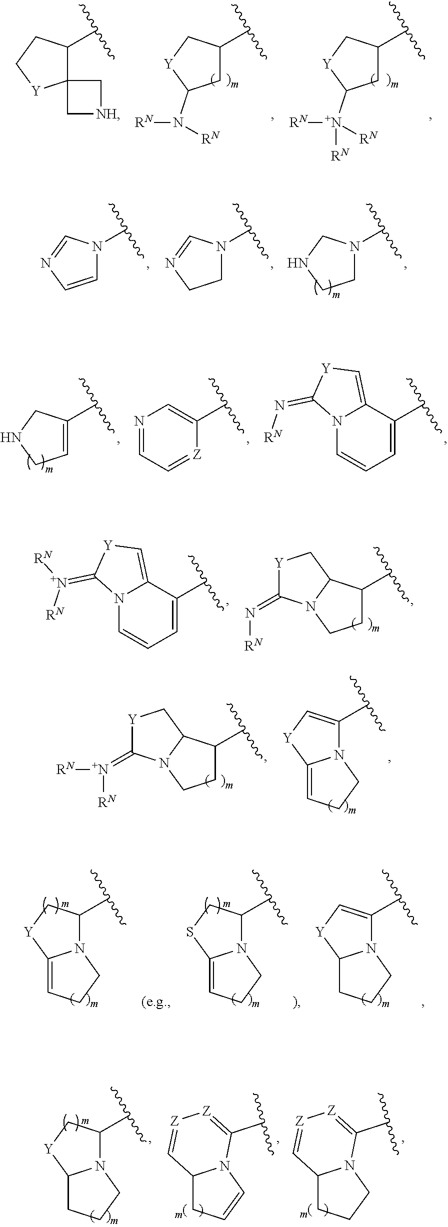

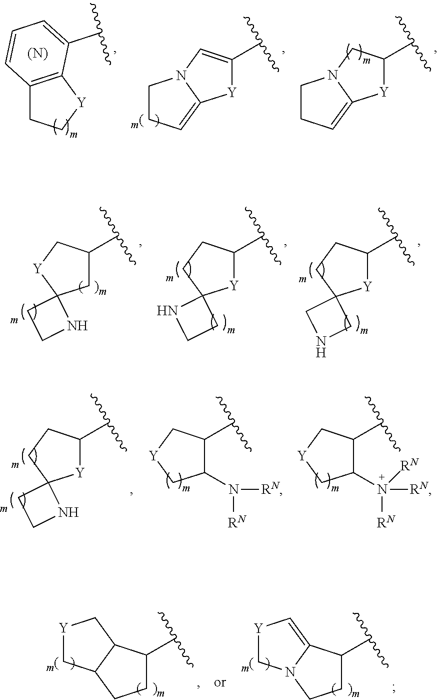

wherein each of the above-depicted groups is optionally substituted with one or more (e.g., one, two or three) groups R^{B1};
wherein each s is independently 0, 1 or 2 (preferably, each s is independently 0 or 1);
wherein each t is independently 0, 1, 2 or 3 (preferably, each t is independently 0, 1 or 2, more preferably 0 or 1);
wherein each m is independently 1, 2 or 3 (preferably, each m is independently 1 or 2);
wherein each ring atom W is independently selected from S, O, SO₂ and NH;
wherein each ring atom Y is independently selected from S, O, SO₂, NH and CH₂;
wherein each ring atom Z is independently C or N;
wherein the symbol “(N)” depicted inside a ring indicates that 0, 1, 2 or 3 ring atom(s) of the respective ring is/are nitrogen ring atom(s) (preferably, the symbol “(N)” depicted inside a ring indicates that 0, 1 or 2 ring atom(s), more preferably 0 or 1 ring atom(s), of the respective ring is/are nitrogen ring atom(s));
wherein the symbol “N” depicted inside a ring indicates that 1, 2 or 3 ring atom(s) of the respective ring is/are nitrogen ring atom(s) (preferably, the symbol “N” depicted inside a ring indicates that 1 or 2 ring atom(s), more preferably 1 ring atom, of the respective ring is/are nitrogen ring atom(s)); and
wherein the symbol “X” depicted inside a 5-membered ring indicates that the corresponding ring is aromatic and that 1, 2 or 3 ring atom(s) of said ring is/are each independently selected from nitrogen, oxygen and sulfur, while the remaining ring atoms are carbon atoms (e.g., said 5-membered ring may be any one of the exemplary or preferred rings described herein above in connection with the 5-membered rings marked with the symbol “X” comprised in the ring groups A7 or A8 to A11).

More preferably, if ring A is a group A2, A3, A4, A5, A7, A8, A9 or A10, then ring B is selected from any one of the following groups:

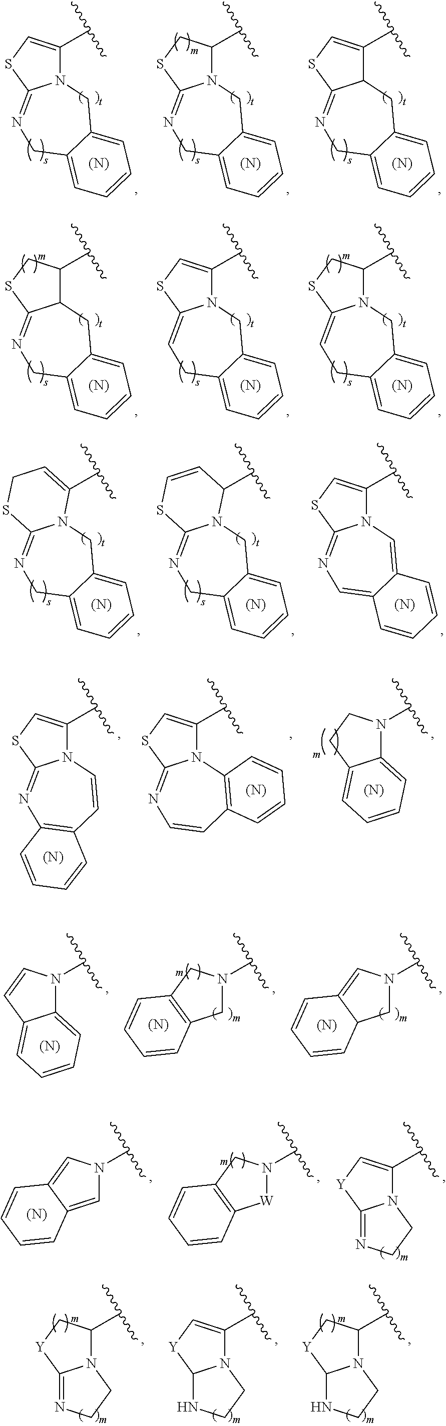

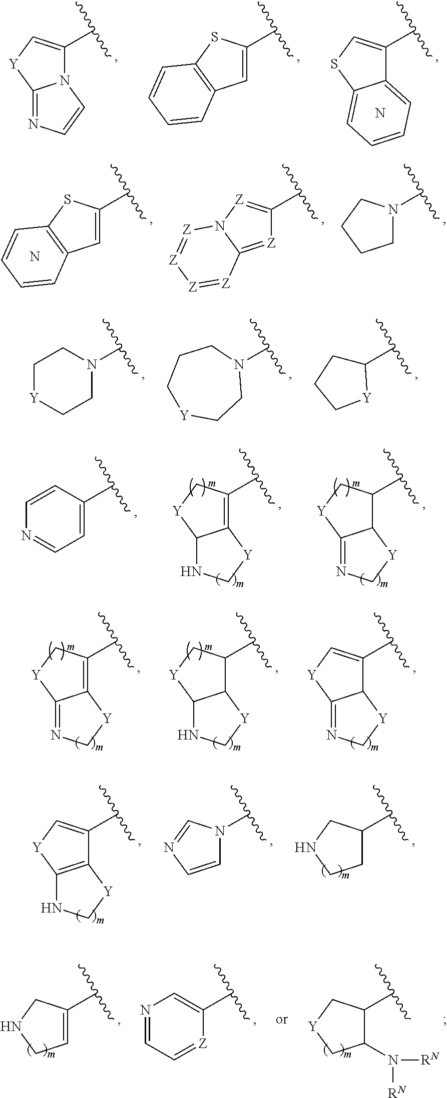

wherein each of the above-depicted groups is optionally substituted with one or more (e.g., one, two or three) groups R^{B1};
wherein each s is independently 0, 1 or 2 (preferably, each s is independently 0 or 1);
wherein each t is independently 0, 1, 2 or 3 (preferably, each t is independently 0, 1 or 2, more preferably 0 or 1);
wherein each m is independently 1, 2 or 3 (preferably, each m is independently 1 or 2);
wherein each ring atom Y is independently selected from S, O, SO₂, NH and CH₂;
wherein each ring atom Z is independently C or N;
wherein the symbol “(N)” depicted inside a ring indicates that 0, 1, 2 or 3 ring atom(s) of the respective ring is/are nitrogen ring atom(s) (preferably, the symbol “(N)” depicted inside a ring indicates that 0, 1 or 2 ring atom(s), more preferably 0 or 1 ring atom(s), of the respective ring is/are nitrogen ring atom(s)); and
wherein the symbol “N” depicted inside a ring indicates that 1, 2 or 3 ring atom(s) of the respective ring is/are nitrogen ring atom(s) (preferably, the symbol “N” depicted inside a ring indicates that 1 or 2 ring atom(s), more preferably 1 ring atom, of the respective ring is/are nitrogen ring atom(s)).

Even more preferably, if ring A is a group A2, A3, A4, A5, A7, A8, A9 or A10 (particularly a group A2), then ring B is selected from any one of the following groups:

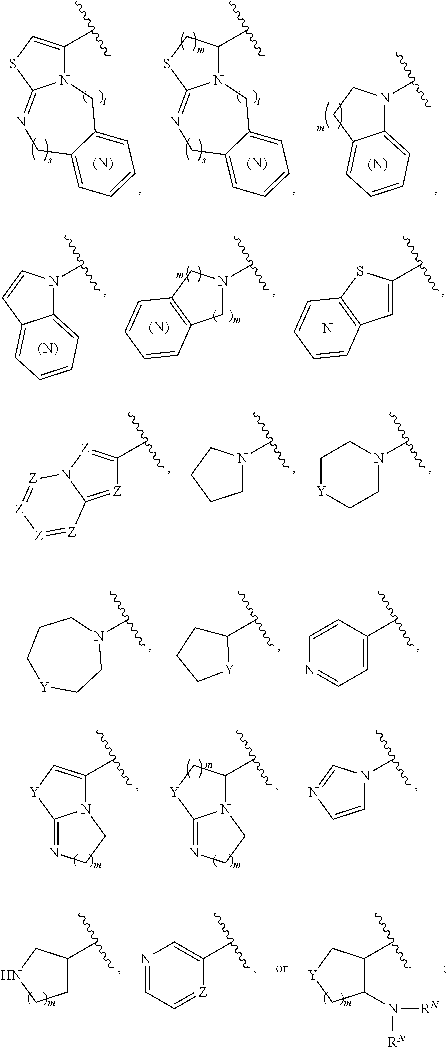

wherein each of the above-depicted groups is optionally substituted with one or more (e.g., one, two or three) groups R^{B1};
wherein each s is independently 0, 1 or 2 (preferably, each s is independently 0 or 1);
wherein each t is independently 0, 1, 2 or 3 (preferably, each t is independently 0, 1 or 2, more preferably 0 or 1);
wherein each m is independently 1, 2 or 3 (preferably, each m is independently 1 or 2);
wherein each ring atom Y is independently selected from S, O, SO₂, NH and CH₂;
wherein each ring atom Z is independently C or N;
wherein the symbol “(N)” depicted inside a ring indicates that 0, 1, 2 or 3 ring atom(s) of the respective ring is/are nitrogen ring atom(s) (preferably, the symbol “(N)” depicted inside a ring indicates that 0, 1 or 2 ring atom(s), more preferably 0 or 1 ring atom(s), of the respective ring is/are nitrogen ring atom(s)); and
wherein the symbol “N” depicted inside a ring indicates that 1, 2 or 3 ring atom(s) of the respective ring is/are nitrogen ring atom(s) (preferably, the symbol “N” depicted inside a ring indicates that 1 or 2 ring atom(s), more preferably 1 ring atom, of the respective ring is/are nitrogen ring atom(s)).

Even more preferably, if ring A is a group A2, A3, A4, A5, A7, A8, A9 or A10, then ring B is selected from any one of the following groups:

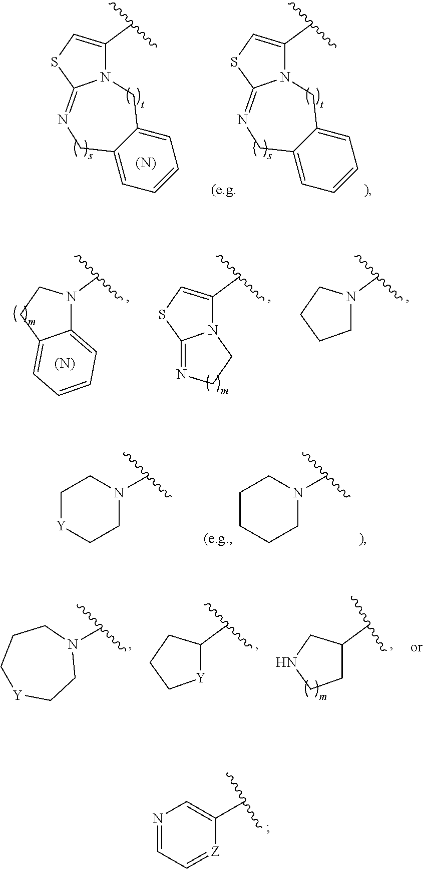

wherein each of the above-depicted groups is optionally substituted with one or more (e.g., one, two or three) groups R^{B1};
wherein each s is independently 0, 1 or 2 (preferably, each s is independently 0 or 1);
wherein each t is independently 0, 1, 2 or 3 (preferably, each t is independently 0, 1 or 2, more preferably 0 or 1);
wherein m is 1, 2 or 3 (preferably 1 or 2; more preferably 1); and
wherein each ring atom Y is independently selected from NH and CH₂;
wherein each ring atom Z is independently C or N;
wherein the symbol “(N)” depicted inside a ring indicates that 0, 1, 2 or 3 ring atom(s) of the respective ring is/are nitrogen ring atom(s) (preferably, the symbol “(N)” depicted inside a ring indicates that 0, 1 or 2 ring atom(s), more preferably 0 or 1 ring atom(s), even more preferably 0 ring atoms, of the respective ring is/are nitrogen ring atom(s)).

Thus, for example, if ring A is a group A2, A3, A4, A5, A7, A8, A9 or A10, then ring B may be selected from any one of the following groups:

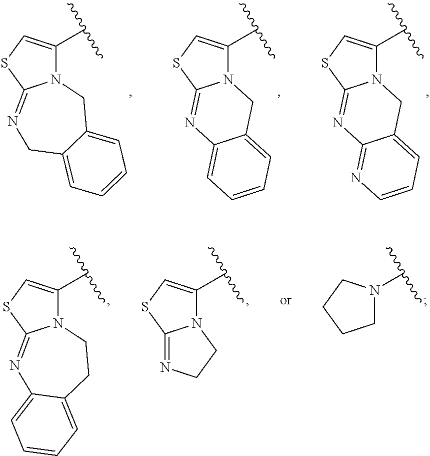 [SMILES: *C1=CSC2=NCc3ccccc3CN12 ; *C1=CSC2=Nc3ccccc3CN12 ; *C1=CSC2=Nc3ncccc3CN12 ; *C1=CSC2=Nc3ccccc3CCN12 ; *C1=CSC2=NCCN12 ; *N1CCCC1]

wherein each one of the above-depicted groups is optionally substituted with one or more (e.g., one, two or three) groups R^{B1}.

Moreover, if ring A is a group A2, A3, A4, A5, A7, A8, A9 or A10, then ring B in formula (I) may also be any one of the specific ring B groups comprised in any one of those compounds of Examples 1 to 200 that have a group A2, A3, A4, A5, A7, A8, A9 or A10 as ring A.

If ring A is a group A6 or A11, then ring B is selected from any one of the following groups:

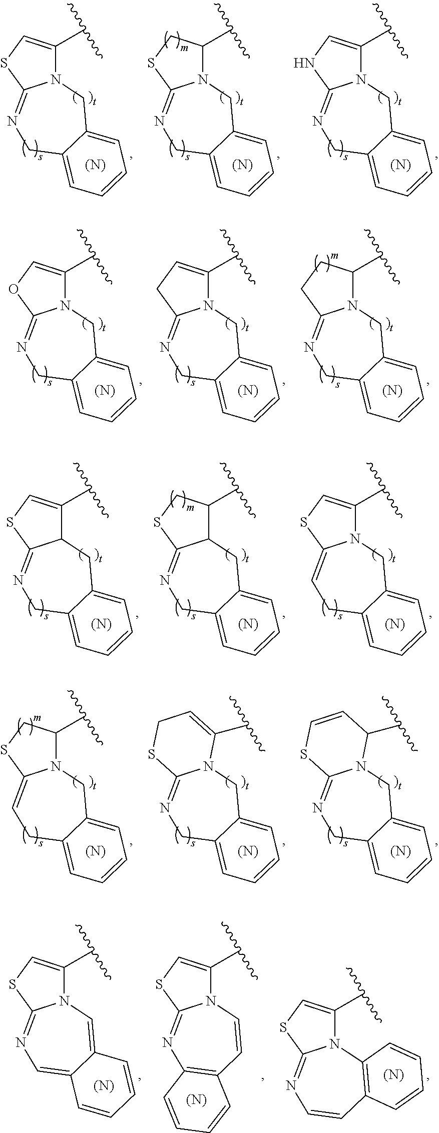

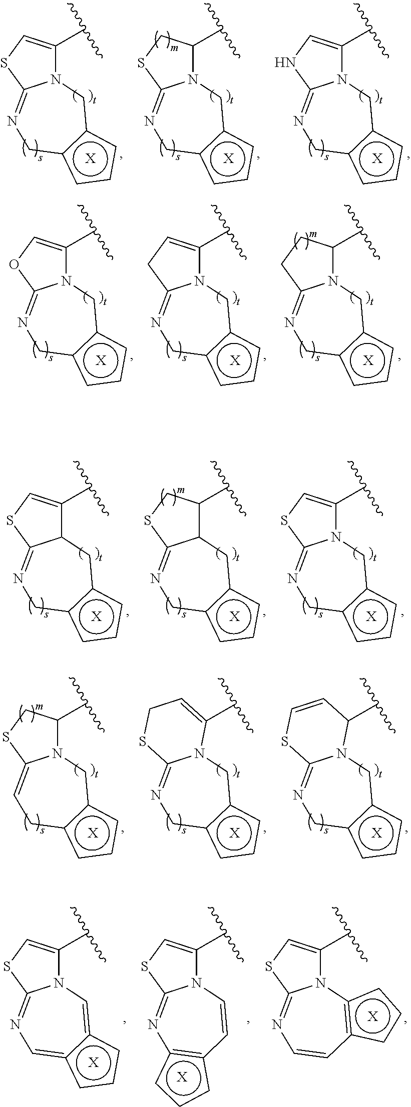

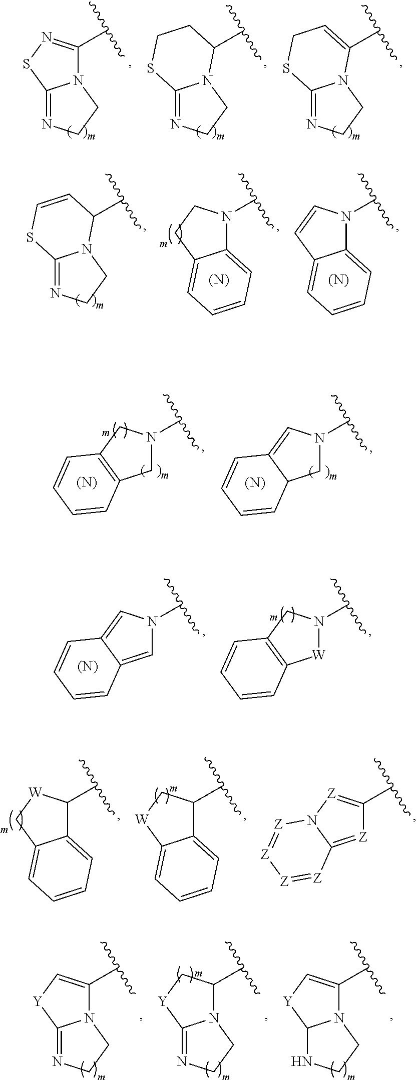

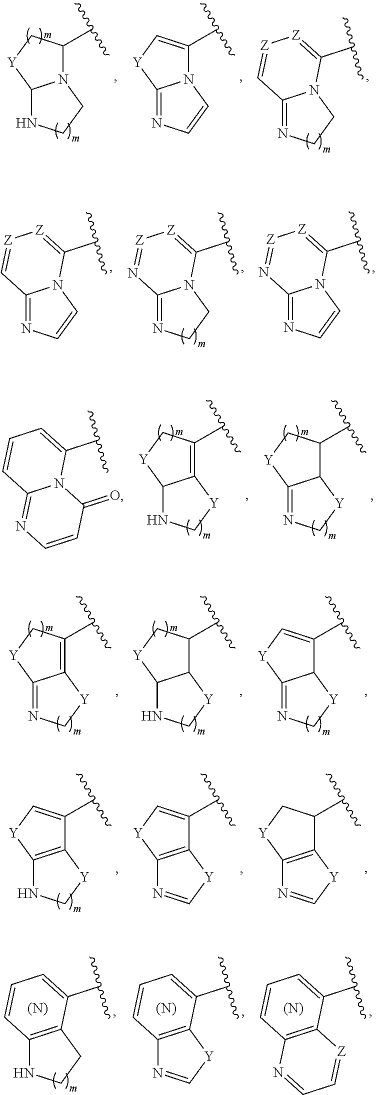

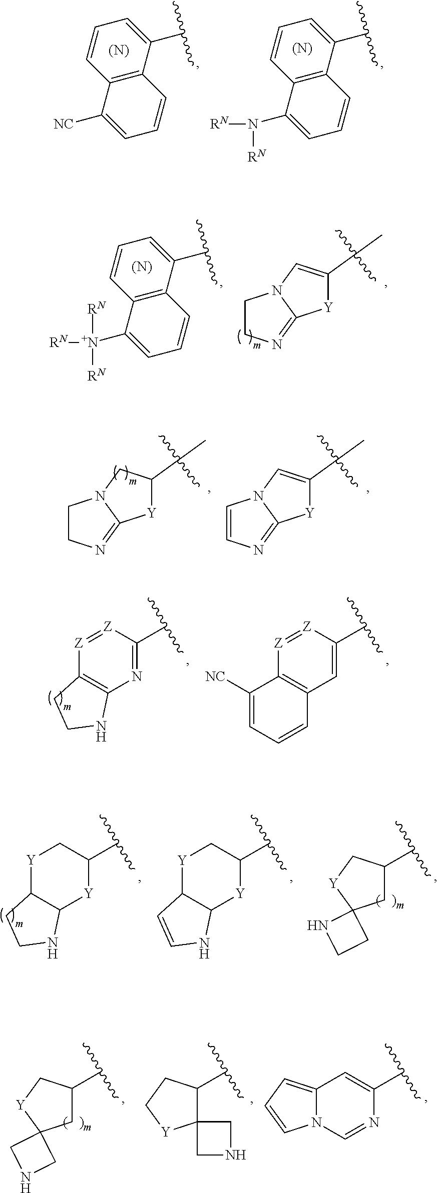

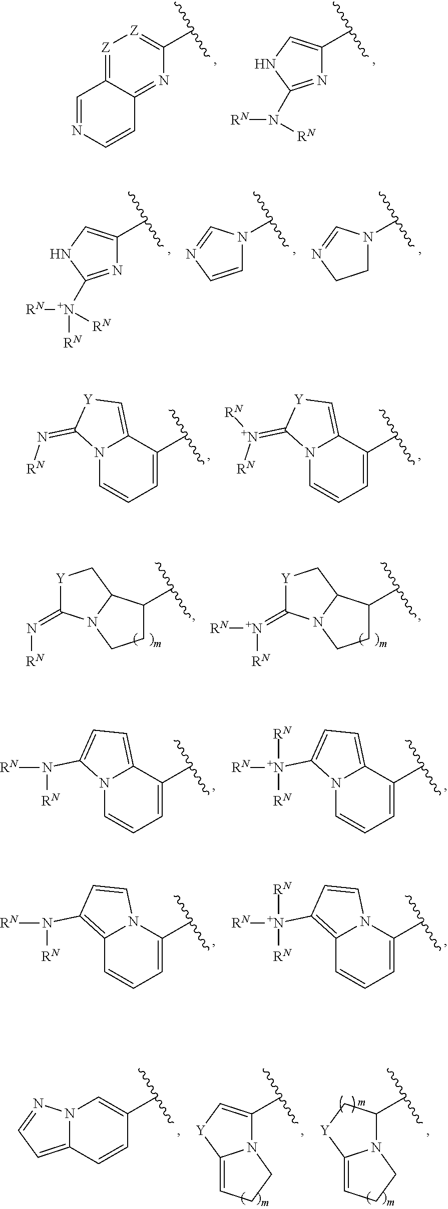

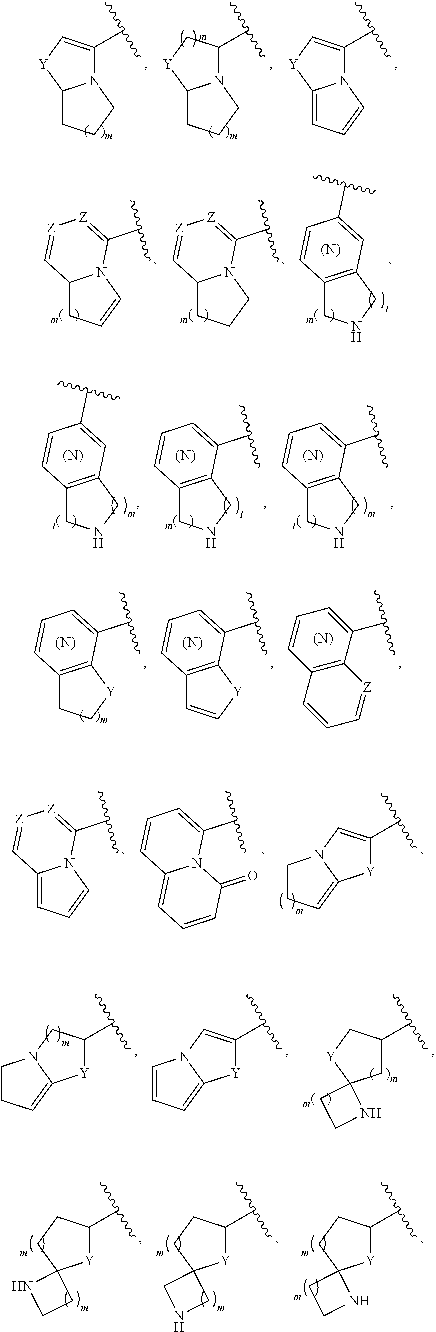

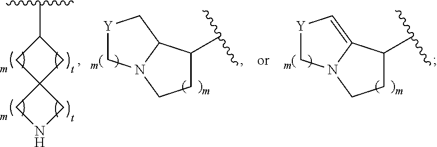

wherein each of the above-depicted groups is optionally substituted with one or more (e.g., one, two or three) groups R^{B1};
wherein each s is independently 0, 1 or 2 (preferably, each s is independently 0 or 1);
wherein each t is independently 0, 1, 2 or 3 (preferably, each t is independently 0, 1 or 2, more preferably 0 or 1);
wherein each m is independently 1, 2 or 3 (preferably, each m is independently 1 or 2);
wherein each ring atom W is independently selected from S, O, SO₂ and NH;
wherein each ring atom Y is independently selected from S, O, SO₂, NH and CH₂;
wherein each ring atom Z is independently C or N;
wherein the symbol “(N)” depicted inside a ring indicates that 0, 1, 2 or 3 ring atom(s) of the respective ring is/are nitrogen ring atom(s) (preferably, the symbol “(N)” depicted inside a ring indicates that 0, 1 or 2 ring atom(s), more preferably 0 or 1 ring atom(s), of the respective ring is/are nitrogen ring atom(s)); and
wherein the symbol “X” depicted inside a 5-membered ring indicates that the corresponding ring is aromatic and that 1, 2 or 3 ring atom(s) of said ring is/are each independently selected from nitrogen, oxygen and sulfur, while the remaining ring atoms are carbon atoms (e.g., said 5-membered ring may be any one of the exemplary or preferred rings described herein above in connection with the 5-membered rings marked with the symbol “X” comprised in the ring groups A7 or A8 to A11).

Preferably, if ring A is a group A6 or A11, then ring B is selected from any one of the following groups:

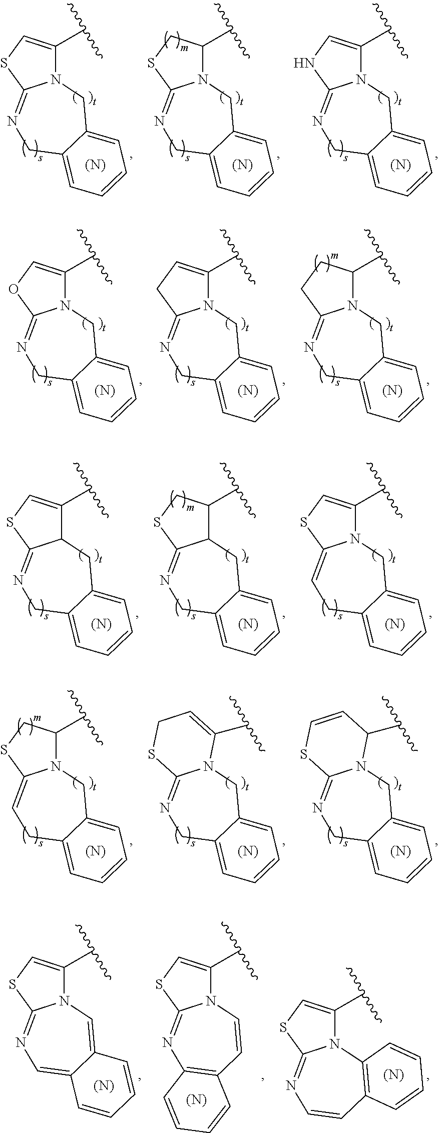

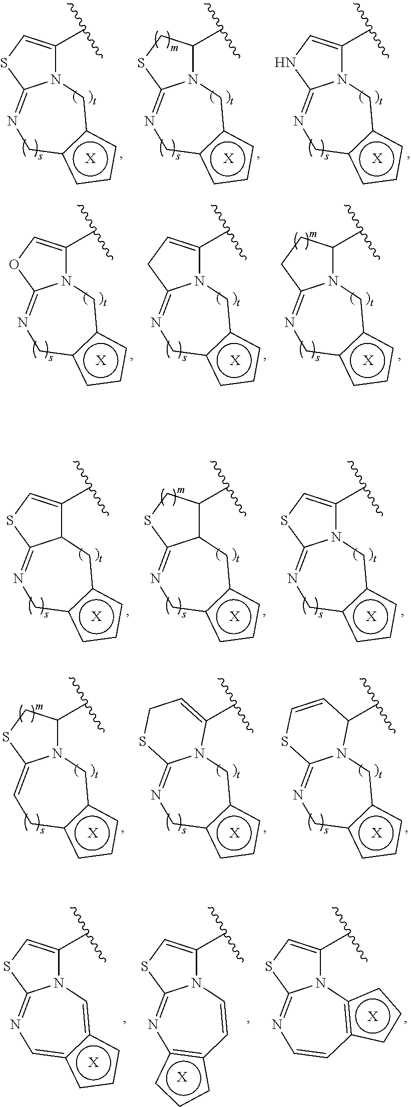

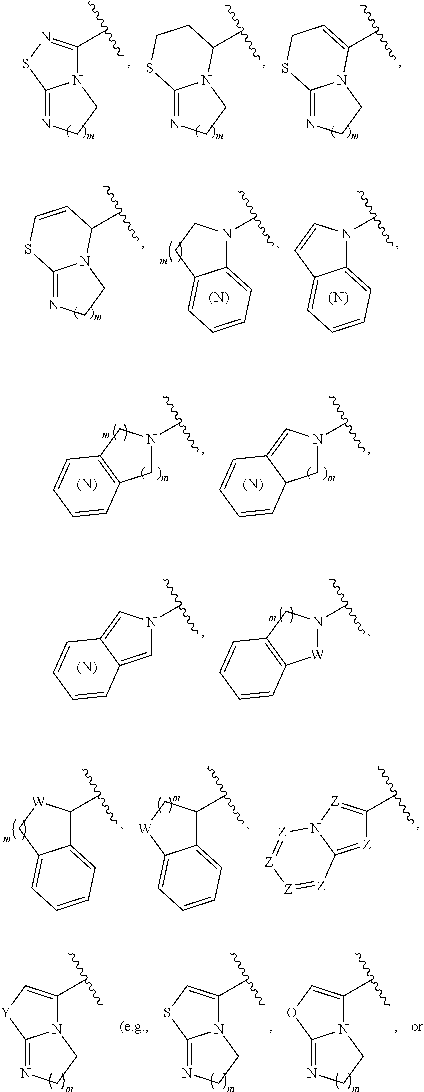

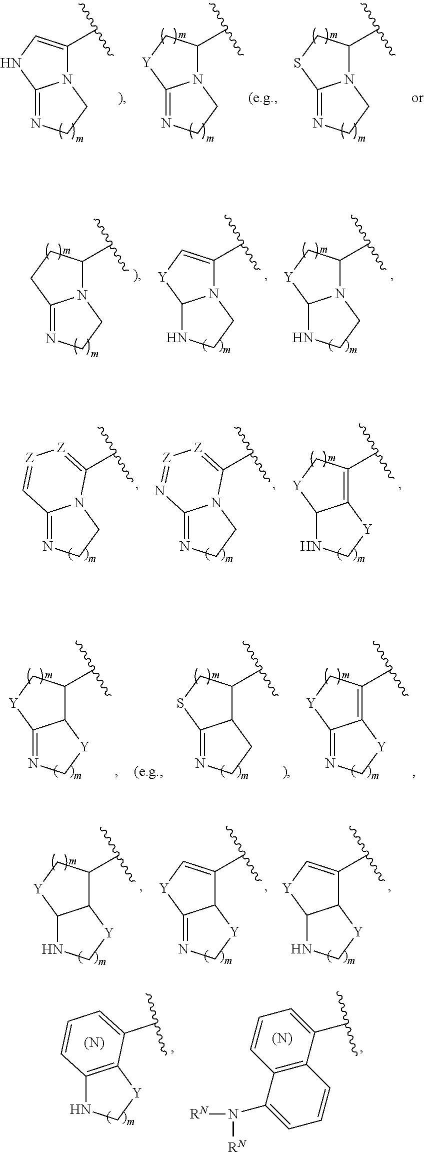

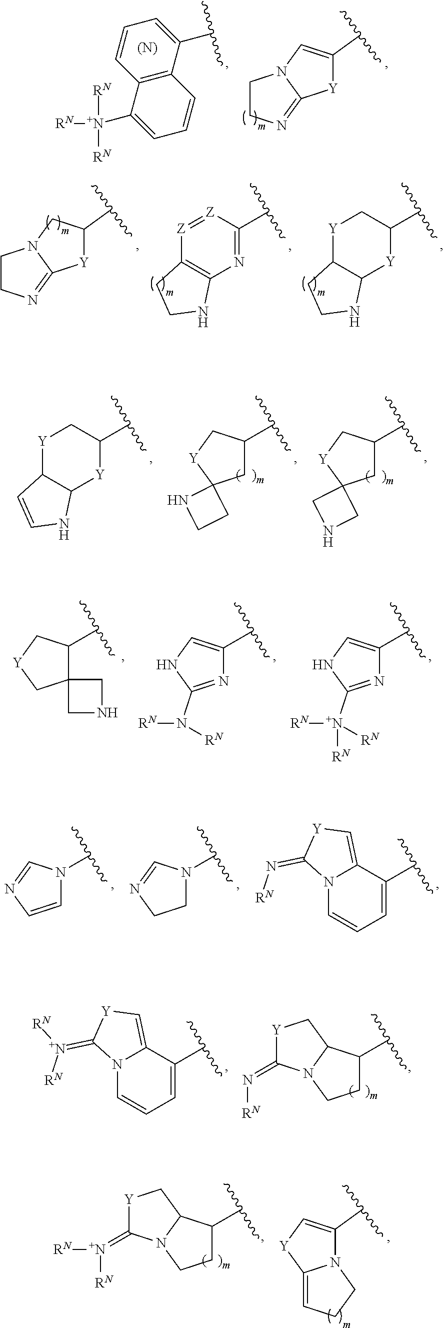

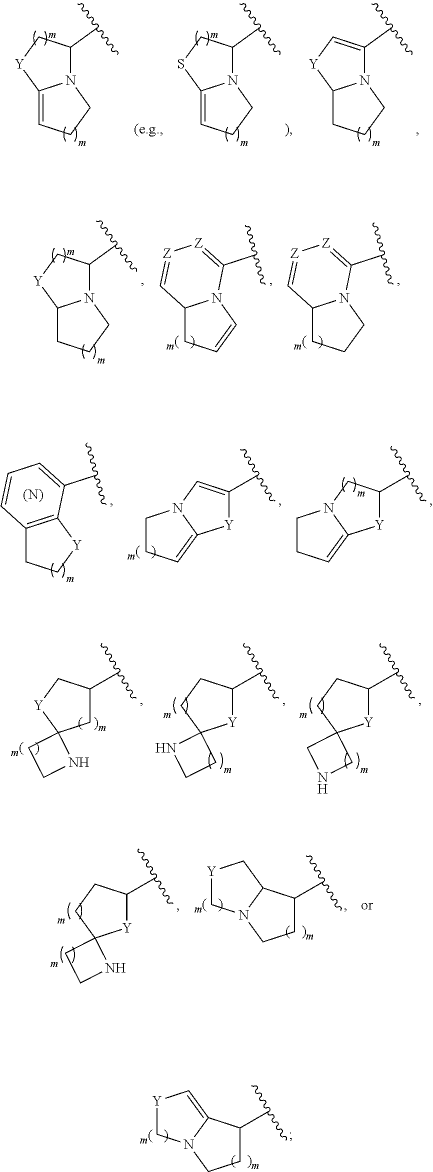

wherein each of the above-depicted groups is optionally substituted with one or more (e.g., one, two or three) groups R^{B1};
wherein each s is independently 0, 1 or 2 (preferably, each s is independently 0 or 1);
wherein each t is independently 0, 1, 2 or 3 (preferably, each t is independently 0, 1 or 2, more preferably 0 or 1);
wherein each m is independently 1, 2 or 3 (preferably, each m is independently 1 or 2);
wherein each ring atom W is independently selected from S, O, SO₂ and NH;
wherein each ring atom Y is independently selected from S, O, SO₂, NH and CH₂;
wherein each ring atom Z is independently C or N;
wherein the symbol “(N)” depicted inside a ring indicates that 0, 1, 2 or 3 ring atom(s) of the respective ring is/are nitrogen ring atom(s) (preferably, the symbol “(N)” depicted inside a ring indicates that 0, 1 or 2 ring atom(s), more preferably 0 or 1 ring atom(s), of the respective ring is/are nitrogen ring atom(s)); and
wherein the symbol “X” depicted inside a 5-membered ring indicates that the corresponding ring is aromatic and that 1, 2 or 3 ring atom(s) of said ring is/are each independently selected from nitrogen, oxygen and sulfur, while the remaining ring atoms are carbon atoms (e.g., said 5-membered ring may be any one of the exemplary or preferred rings described herein above in connection with the 5-membered rings marked with the symbol “X” comprised in the ring groups A7 or A8 to A11).

More preferably, if ring A is a group A6 or A11, then ring B is selected from any one of the following groups:

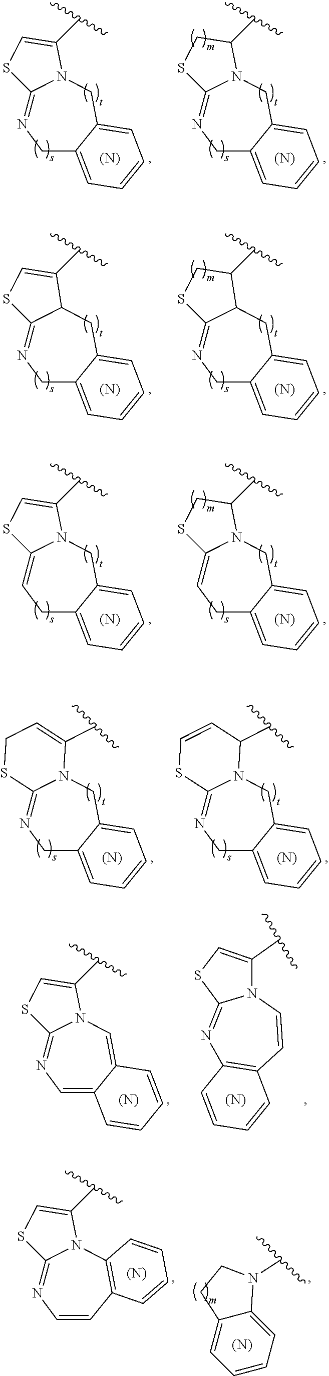

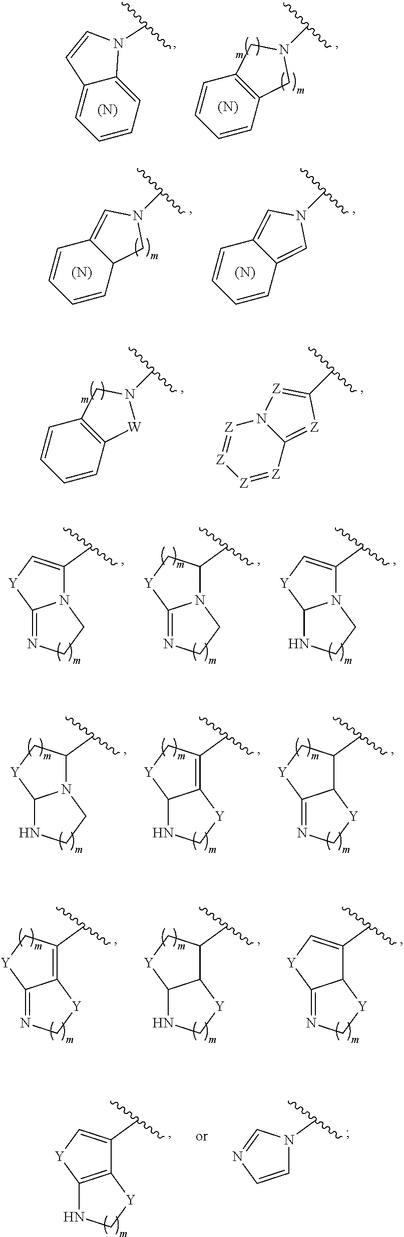

wherein each of the above-depicted groups is optionally substituted with one or more (e.g., one, two or three) groups R^{B1};
wherein each s is independently 0, 1 or 2 (preferably, each s is independently 0 or 1);
wherein each t is independently 0, 1, 2 or 3 (preferably, each t is independently 0, 1 or 2, more preferably 0 or 1);
wherein each m is independently 1, 2 or 3 (preferably, each m is independently 1 or 2);
wherein each ring atom W is independently selected from S, O, SO₂ and NH;
wherein each ring atom Y is independently selected from S, O, SO₂, NH and CH₂;
wherein each ring atom Z is independently C or N; and
wherein the symbol “(N)” depicted inside a ring indicates that 0, 1, 2 or 3 ring atom(s) of the respective ring is/are nitrogen ring atom(s) (preferably, the symbol “(N)” depicted inside a ring indicates that 0, 1 or 2 ring atom(s), more preferably 0 or 1 ring atom(s), of the respective ring is/are nitrogen ring atom(s)).

Even more preferably, if ring A is a group A6 or A11, then ring B is selected from any one of the following groups:

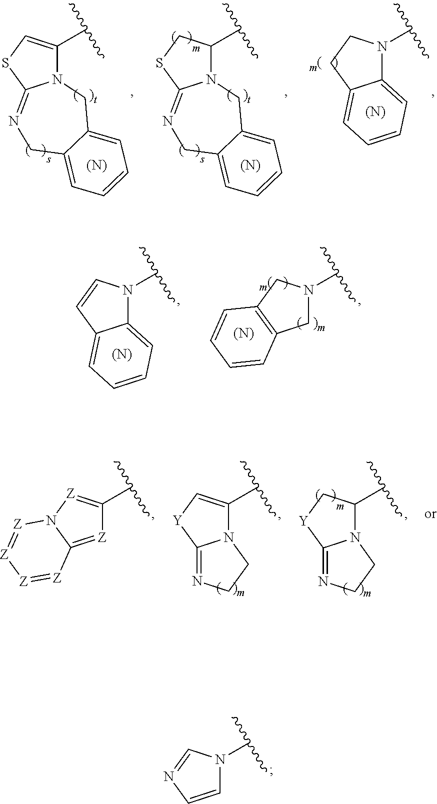

wherein each of the above-depicted groups is optionally substituted with one or more (e.g., one, two or three) groups R^{B1};
wherein each s is independently 0, 1 or 2 (preferably, each s is independently 0 or 1);
wherein each t is independently 0, 1, 2 or 3 (preferably, each t is independently 0, 1 or 2, more preferably 0 or 1);
wherein each m is independently 1, 2 or 3 (preferably, each m is independently 1 or 2);
wherein each ring atom Y is independently selected from S, O, SO₂, NH and CH₂;
wherein each ring atom Z is independently C or N; and
wherein the symbol “(N)” depicted inside a ring indicates that 0, 1, 2 or 3 ring atom(s) of the respective ring is/are nitrogen ring atom(s) (preferably, the symbol “(N)” depicted inside a ring indicates that 0, 1 or 2 ring atom(s), more preferably 0 or 1 ring atom(s), of the respective ring is/are nitrogen ring atom(s)).

Even more preferably, if ring A is a group A6 or A11, then ring B is selected from any one of the following groups:

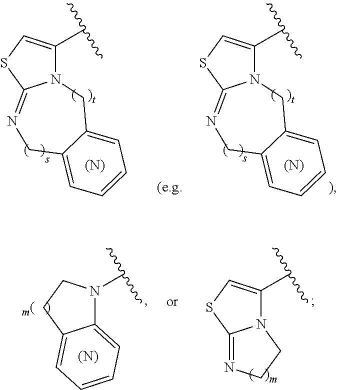

wherein each of the above-depicted groups is optionally substituted with one or more (e.g., one, two or three) groups R^{B1};
wherein each s is independently 0, 1 or 2 (preferably, each s is independently 0 or 1);
wherein each t is independently 0, 1, 2 or 3 (preferably, each t is independently 0, 1 or 2, more preferably 0 or 1);
wherein m is 1, 2 or 3 (preferably 1 or 2); and
wherein the symbol “(N)” depicted inside a ring indicates that 0, 1, 2 or 3 ring atom(s) of the respective ring is/are nitrogen ring atom(s) (preferably, the symbol “(N)” depicted inside a ring indicates that 0, 1 or 2 ring atom(s), more preferably 0 or 1 ring atom(s), even more preferably 0 ring atoms, of the respective ring is/are nitrogen ring atom(s)).

Thus, for example, if ring A is a group A6 or A11, then ring B may be selected from any one of the following groups:

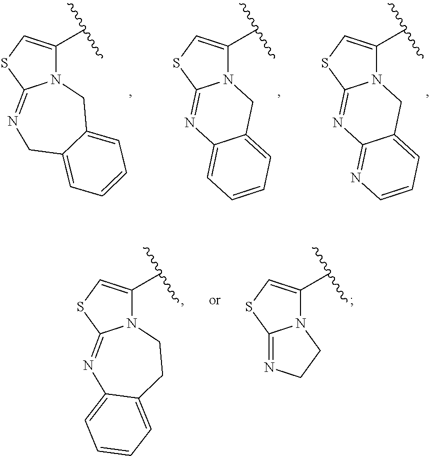 [SMILES: *C1=CSC2=NCc3ccccc3CN12 ; *C1=CSC2=Nc3ccccc3CN12 ; *C1=CSC2=Nc3ncccc3CN12 ; *C1=CSC2=Nc3ccccc3CCN12 ; *C1=CSC2=NCCN12]

wherein each one of the above-depicted groups is optionally substituted with one or more (e.g., one, two or three) groups R^{B1}.

Moreover, if ring A is a group A6 or A11, then ring B in formula (I) may also be any one of the specific ring B groups comprised in any one of those compounds of Examples 1 to 200 that have a group A6 or A11 as ring A.

R^{A1} is selected from hydrogen, C₁₋₅ alkyl, C₂₋₅ alkenyl, C₂₋₅ alkynyl, —CO(C₁₋₅ alkyl), —COO(C₁₋₅ alkyl), carbocyclyl, and heterocyclyl, wherein said alkyl, said alkenyl, said alkynyl, the alkyl moiety in said —CO(C₁₋₅ alkyl), and the alkyl moiety in said —COO(C₁₋₅ alkyl) are each optionally substituted with one or more (e.g., one, two or three) groups R^{Alk}, and further wherein said carbocyclyl and said heterocyclyl are each optionally substituted with one or more (e.g., one, two or three) groups R^{Cyc}.

Preferably, R^{A1} is selected from hydrogen, C₁₋₅ alkyl, —CO(C₁₋₅ alkyl), carbocyclyl, and heterocyclyl, wherein said alkyl and the alkyl moiety in said —CO(C₁₋₅ alkyl) are each optionally substituted with one or more groups R^{Alk}, and further wherein said carbocyclyl and said heterocyclyl are each optionally substituted with one or more groups R^{Cyc}.

More preferably, R^{A1} is selected from hydrogen, C₁₋₅ alkyl, cycloalkyl, heterocycloalkyl, —(C₀₋₅ alkylene)-aryl, and —(C₀₋₅ alkylene)-heteroaryl, wherein said cycloalkyl and said heterocycloalkyl are each optionally substituted with one or more groups R^{Cyc}, and further wherein the aryl moiety in said —(CO₅ alkylene)-aryl and the heteroaryl moiety in said —(C₀₋₅ alkylene)-heteroaryl are each optionally substituted with one or more groups selected independently from C₁₋₅ alkyl, C₂₋₅ alkenyl, C₂₋₅ alkynyl, halogen, C₁₋₅ haloalkyl, —O(C₁₋₅ haloalkyl), —CN, —OH, —O(C₁₋₅ alkyl), —SH, —S(C₁₋₅ alkyl), —NH₂, —NH(C₁₋₅ alkyl), and —N(C₁₋₅ alkyl)(C₁₋₅ alkyl). Even more preferably, R^{A1} is selected from hydrogen, C₁₋₅ alkyl (e.g., methyl or ethyl), and cycloalkyl (e.g., cyclopropyl, cyclopentyl, or cyclohexyl), wherein said cycloalkyl is optionally substituted with one or more groups R^{Cyc}. Yet even more preferably, R^{A1} is hydrogen or C₁₋₅ alkyl (e.g., methyl). Still more preferably, R^{A1} is hydrogen.

Each R^{A2} is independently selected from C₁₋₅ alkyl, C₂₋₅ alkenyl, C₂₋₅ alkynyl, —(C₀₋₅ alkylene)-R^{A21}, —(C₂₋₅ alkenylene)-R^{A21}, and —(C₂₋₅ alkynylene)-R^{A21}, wherein said alkyl, said alkenyl, said alkynyl, said alkylene, said alkenylene, and said alkynylene are each optionally substituted with one or more (e.g., one, two or three) groups R^{Alk}, and further wherein one or more (e.g., one, two or three) —CH₂— units comprised in said alkylene, said alkenylene, or said alkynylene are each optionally replaced by a group independently selected from —O—, —NH—, —N(C₁₋₅ alkyl)-, —CO—, —S—, —SO—, and —SO₂—;
any two groups R^{A2}, which are attached to the same ring atom of ring A, may also be mutually joined to form, together with the ring atom that they are attached to, a cycloalkyl or a heterocycloalkyl, wherein said cycloalkyl or said heterocycloalkyl is optionally substituted with one or more (e.g., one, two or three) groups R^{Cyc};
any two groups R^{A2}, which are attached to distinct ring atoms of ring A, may also be mutually joined to form a C₁₋₅ alkylene which is optionally substituted with one or more (e.g., one, two or three) groups R^{Cyc}, and wherein one or more (e.g., one, two or three) —CH₂— units comprised in said alkylene are each optionally replaced by a group independently selected from —O—, —NH—, —N(C₁₋₅ alkyl)-, —CO—, —S—, —SO—, —SO₂—, and phen-1,2-diyl, wherein said phen-1,2-diyl is optionally substituted with one or more (e.g., one, two or three) groups R^{Cyc}; and
any one group R^{A2} may also be mutually joined with R^{A1} to form a C₁₋₅ alkylene which is optionally substituted with one or more (e.g., one, two or three) groups R^{Cyc}, and wherein one or more (e.g., one, two or three) —CH₂— units comprised in said alkylene are each optionally replaced by a group independently selected from —O—, —NH—, —N(C₁₋₅ alkyl)-, —CO—, —S—, —SO—, —SO₂—, and phen-1,2-diyl, wherein said phen-1,2-diyl is optionally substituted with one or more (e.g., one, two or three) groups R^{Cyc}.

Each R^{A21} is independently selected from halogen, C₁₋₅ haloalkyl, —O(C₁₋₅ haloalkyl), —CN, —OR^{A22}, —NR^{A22}R^{A22}, —NR^{A22}OR^{A22}, —COR^{A22}, —COOR^{A22}, —OCOR^{A22}, —CONR^{A22}R^{A22}, —NR^{A22}COR^{A22}, —NR^{A22}COOR^{A22}, —OCONR^{A22}R^{A22}, —SR^{A22}, —SOR^{A22}, —SO₂R^{A22}, —SO₂NR^{A22}R^{A22}, —NR^{A22}SO₂R^{A22}, —SO₃R^{A22}, —NO₂, carbocyclyl, and heterocyclyl, wherein said carbocyclyl and said heterocyclyl are each optionally substituted with one or more (e.g., one, two or three) groups R^{Cyc}.

Preferably, each R^{A21} is independently selected from halogen, C₁₋₅ haloalkyl, —O(C₁₋₅ haloalkyl), —CN, —OR^{A22}, —NR^{A22}R^{A22}, —COR^{A22}, —COOR^{A22}, —OCOR^{A22}, —CONR^{A22}R^{A22}, —NR^{A22}COR^{A22}, —SR^{A22}, —SOR^{A22}, —SO₂R^{A22}, —SO₂NR^{A22}R^{A22}, —NR^{A22}SO₂R^{A22}, cycloalkyl, aryl, heterocycloalkyl, and heteroaryl, wherein said cycloalkyl, said aryl, said heterocycloalkyl and said heteroaryl are each optionally substituted with one or more (e.g., one, two or three) groups R^{Cyc}. More preferably, each R^{A21} is independently selected from halogen, C₁₋₅ haloalkyl, —O(C₁₋₅ haloalkyl), —CN, —OH, —O(C₁₋₅ alkyl), —O(C₁₋₅ alkylene)-OH, —O(C₁₋₅ alkylene)-O(C₁₋₅ alkyl), —NH₂, —NH(C₁₋₅ alkyl), —N(C₁₋₅ alkyl)(C₁₋₅ alkyl), —CHO, —CO(C₁₋₅ alkyl), —COOH, —COO(C₁₋₅ alkyl), —O—CO(C₁₋₅ alkyl), —CO—NH₂, —CO—NH(C₁₋₅ alkyl), —CO—N(C₁₋₅ alkyl)(C₁₋₅ alkyl), —NH—CO(C₁₋₅ alkyl), —N(C₁₋₅ alkyl)-CO(C₁₋₅ alkyl), —SH, —S(C₁₋₅ alkyl), —SO—(C₁₋₅ alkyl), —SO₂—(C₁₋₅ alkyl), —SO₂—NH₂, —SO₂—NH(C₁₋₅ alkyl), —SO₂—N(C₁₋₅ alkyl)(C_{1- 5} alkyl), —NH—SO₂—(C₁₋₅ alkyl), —N(C₁₋₅ alkyl)-SO₂—(C₁₋₅ alkyl), cycloalkyl, aryl, heterocycloalkyl, and heteroaryl, wherein said cycloalkyl, said aryl, said heterocycloalkyl and said heteroaryl are each optionally substituted with one or more groups R^{Cyc}. Even more preferably, each R^{A21} is independently selected from halogen, C₁₋₅ haloalkyl (e.g., —CF₃), —O(C₁₋₅ haloalkyl), —CN, —OH, —O(C₁₋₅ alkyl), —NH₂, —NH(C₁₋₅ alkyl), —N(C₁₋₅ alkyl)(C₁₋₅ alkyl), cycloalkyl, aryl, heterocycloalkyl, and heteroaryl, wherein said cycloalkyl, said aryl, said heterocycloalkyl and said heteroaryl are each optionally substituted with one or more groups R^{Cyc}. Yet even more preferably, each R^{A21} is independently selected from cycloalkyl, aryl, heterocycloalkyl, and heteroaryl, wherein said cycloalkyl, said aryl, said heterocycloalkyl and said heteroaryl are each optionally substituted with one or more groups R^{Cyc}. Still more preferably, each R^{A21} is independently aryl or heteroaryl, wherein said aryl and said heteroaryl are each optionally substituted with one or more groups R^{Cyc}.

Each R^{A22} is independently selected from hydrogen, C₁₋₅ alkyl, C₂₋₅ alkenyl, C₂₋₅ alkynyl, carbocyclyl, and heterocyclyl, wherein said alkyl, said alkenyl and said alkynyl are each optionally substituted with one or more (e.g., one, two or three) groups R^{Alk}, and further wherein said carbocyclyl and said heterocyclyl are each optionally substituted with one or more (e.g., one, two or three) groups R^{Cyc}.

Preferably, each R^{A22} is independently selected from hydrogen and C₁₋₅ alkyl, wherein said alkyl is optionally substituted with one or more groups R^{Alk}. More preferably, each R^{A22} is independently selected from hydrogen and C₁₋₅ alkyl (e.g., methyl or ethyl).

As explained above, each R^{A2} may be independently selected from C₁₋₅ alkyl, C₂₋₅ alkenyl, C₂₋₅ alkynyl, —(C₀₋₅ alkylene)-R^{A21}, —(C₂₋₅ alkenylene)-R^{A21}, and —(C₂₋₅ alkynylene)-R^{A21}, wherein said alkyl, said alkenyl, said alkynyl, said alkylene, said alkenylene, and said alkynylene are each optionally substituted with one or more groups R^{Alk}, and further wherein one or more —CH₂— units comprised in said alkylene, said alkenylene, or said alkynylene are each optionally replaced by a group independently selected from —O—, —NH—, —N(C₁₋₅ alkyl)-, —CO—, —S—, —SO—, and —SO₂—. In this case, and in accordance with the above definition of R^{A21}, it is preferred that each R^{A2} is independently selected from C₁₋₅ alkyl, C₂₋₅ alkenyl, C₂₋₅ alkynyl, halogen, C₁₋₅ haloalkyl, —(C₀₋₅ alkylene)-O(C₁₋₅ haloalkyl), —(C₀₋₅ alkylene)-CN, —(C₀₋₅ alkylene)-OH, —(C₀₋₅ alkylene)-O(C₁₋₅ alkyl), —(C₀₋₅ alkylene)-O(C₁₋₅ alkylene)-OH, —(C₀₋₅ alkylene)-O(C₁₋₅ alkylene)-O(C₁₋₅ alkyl), —(C₀₋₅ alkylene)-NH₂, —(C₀₋₅ alkylene)-NH(C₁₋₅ alkyl), —(C₀₋₅ alkylene)-N(C₁₋₅ alkyl)(C₁₋₅ alkyl), —(C₀₋₅ alkylene)-CHO, —(C₀₋₅ alkylene)-CO(C₁₋₅ alkyl), —(C₀₋₅ alkylene)-COOH, —(C₀₋₅ alkylene)-COO(C₁₋₅ alkyl), —(C₀₋₅ alkylene)-O—CO(C₁₋₅ alkyl), —(C₀₋₅ alkylene)-CO—NH₂, —(C₀₋₅ alkylene)-CO—NH(C₁₋₅ alkyl), —(C₀₋₅ alkylene)-CO—N(C₁₋₅ alkyl)(C₁₋₅ alkyl), —(C₀₋₅ alkylene)-NH—CO(C₁₋₅ alkyl), —(C₀₋₅ alkylene)-N(C₁₋₅ alkyl)-CO(C₁₋₅ alkyl), —(C₀₋₅ alkylene)-SH, —(C₀₋₅ alkylene)-S(C₁₋₅ alkyl), —(C₀₋₅ alkylene)-SO—(C₁₋₅ alkyl), —(C₀₋₅ alkylene)-SO₂—(C₁₋₅ alkyl), —(C₀₋₅ alkylene)-SO₂—NH₂, —(C₀₋₅ alkylene)-SO₂—NH(C₁₋₅ alkyl), —(C₀₋₅ alkylene)-SO₂—N(C₁₋₅ alkyl)(C₁₋₅ alkyl), —(C₀₋₅ alkylene)-NH—SO₂—(C₁₋₅ alkyl), —(C₀₋₅ alkylene)-N(C₁₋₅ alkyl)-SO₂—(C₁₋₅ alkyl), —(C₀₋₅ alkylene)-cycloalkyl, —(C₀₋₅ alkylene)-aryl, —(C₀₋₅ alkylene)-heterocycloalkyl, and —(C₀₋₅ alkylene)-heteroaryl, wherein the cycloalkyl moiety in said —(C₀₋₅ alkylene)-cycloalkyl, the aryl moiety in said —(C₀₋₅ alkylene)-aryl, the heterocycloalkyl moiety in said —(C₀₋₅ alkylene)-heterocycloalkyl, and the heteroaryl moiety in said —(CO₅ alkylene)-heteroaryl are each optionally substituted with one or more (e.g., one, two or three) groups R^{Cyc}. More preferably, each R^{A2} is independently selected from C₁₋₅ alkyl, halogen, C₁₋₅ haloalkyl (e.g., —CF₃), —(C₀₋₃ alkylene)-O(C₁₋₅ haloalkyl) (e.g., —OCF₃), —(C₀₋₃ alkylene)-CN, —(C₀₋₃ alkylene)-OH, —(C₀₋₃ alkylene)-O(C₁₋₅ alkyl), —(C₀₋₃ alkylene)-NH₂, —(C₀₋₃ alkylene)-NH(C₁₋₅ alkyl), —(C₀₋₃ alkylene)-N(C₁₋₅ alkyl)(C₁₋₅ alkyl), —(C₀₋₃ alkylene)-cycloalkyl, —(C₀₋₃ alkylene)-aryl, —(C₀₋₃ alkylene)-heterocycloalkyl, and —(C₀₋₃ alkylene)-heteroaryl, wherein the cycloalkyl moiety in said —(C₀₋₃ alkylene)-cycloalkyl, the aryl moiety in said —(C₀₋₃ alkylene)-aryl, the heterocycloalkyl moiety in said —(C₀₋₃ alkylene)-heterocycloalkyl, and the heteroaryl moiety in said —(C₀₋₃ alkylene)-heteroaryl are each optionally substituted with one or more groups R^{Cyc}. Even more preferably, each R^{A2} is independently selected from C₁₋₅ alkyl (e.g., methyl, butyl or pentyl), —(C₀₋₃ alkylene)-cycloalkyl, —(C₀₋₃ alkylene)-aryl, —(C₀₋₃ alkylene)-heterocycloalkyl, and —(C₀₋₃ alkylene)-heteroaryl, wherein the cycloalkyl moiety in said —(C₀₋₃ alkylene)-cycloalkyl, the aryl moiety in said —(C₀₋₃ alkylene)-aryl, the heterocycloalkyl moiety in said —(C₀₋₃ alkylene)-heterocycloalkyl, and the heteroaryl moiety in said —(C₀₋₃ alkylene)-heteroaryl are each optionally substituted with one or more groups R^{Cyc}. Yet even more preferably, each R^{A2} is independently selected from C₁₋₅ alkyl, —(C₀₋₃ alkylene)-aryl and —(C₀₋₃ alkylene)-heteroaryl, wherein the aryl moiety in said —(C₀₋₃ alkylene)-aryl and the heteroaryl moiety in said —(C₀₋₃ alkylene)-heteroaryl are each optionally substituted with one or more groups R^{Cyc}. Specific preferred examples of R^{A2} include, in particular, methyl, n-butyl, cyclohexyl, —(C₀₋₃ alkylene)-phenyl (e.g., phenyl or benzyl), —(C₀₋₃ alkylene)-phenyl-halogen (e.g., 4-chlorophenyl or 4-chlorobenzyl), or —(C₀₋₃ alkylene)-imidazolyl (e.g., 3-(imidazol-5-yl)propyl).

As also explained above, any two groups R^{A2}, which are attached to the same ring atom of ring A, may be mutually joined to form, together with the ring atom that they are attached to, a cycloalkyl or a heterocycloalkyl, wherein said cycloalkyl or said heterocycloalkyl is optionally substituted with one or more groups R^{Cyc}. It is preferred that said cycloalkyl or said heterocycloalkyl (which is optionally substituted with one or more R^{Cyc}) has 3 to 8 ring members, more preferably 3, 4, 5 or 6 ring members. Moreover, it is preferred that said cycloalkyl or said heterocycloalkyl is monocyclic. Accordingly, it is particularly preferred that said cycloalkyl (which is formed from any two groups R^{A2} that are attached to the same ring atom of ring A, and which is optionally substituted with one or more groups R^{Cyc}) is a monocyclic C₃₋₃ cycloalkyl, more preferably a monocyclic C₃₋₅ cycloalkyl (e.g., cyclopropyl). It is furthermore particularly preferred that said heterocycloalkyl (which is formed from any two groups R^{A2} that are attached to the same ring atom of ring A, and which is optionally substituted with one or more groups R^{Cyc}) is a monocyclic 3 to 8-membered heterocycloalkyl, more preferably a monocyclic 4 to 6-membered heterocycloalkyl (e.g., tetrahydrofuranyl).

As also explained above, any two groups R^{A2}, which are attached to distinct ring atoms of ring A, may be mutually joined to form a C₁₋₅ alkylene which is optionally substituted with one or more groups R^{Cyc}, and wherein one or more —CH₂— units comprised in said alkylene are each optionally replaced by a group independently selected from —O—, —NH—, —N(C₁₋₅ alkyl)-, —CO—, —S—, —SO—, —SO₂—, and phen-1,2-diyl, wherein said phen-1,2-diyl is optionally substituted with one or more groups R^{Cyc}. It is preferred that one or more (e.g., one or two) —CH₂— units comprised in said alkylene are each optionally replaced by a group independently selected from —O—, —NH—, —N(C₁₋₅ alkyl)-, and —CO—. The C₁₋₅ alkylene is preferably a linear C₁₋₅ alkylene (e.g., a linear C₃₋₅ alkylene), more preferably a group —(CH₂)₁₋₅— (e.g., —(CH₂)₃₋₅—).

Moreover, as also explained above, any one group R^{A2} may be mutually joined with R^{A1} to form a C₁₋₅ alkylene which is optionally substituted with one or more groups R^{Cyc}, and wherein one or more —CH₂— units comprised in said alkylene are each optionally replaced by a group independently selected from —O—, —NH—, —N(C₁₋₅ alkyl)-, —CO—, —S—, —SO—, —SO₂—, and phen-1,2-diyl, wherein said phen-1,2-diyl is optionally substituted with one or more groups R^{Cyc}. It is preferred that one or more (e.g., one or two) —CH₂— units comprised in said alkylene are each optionally replaced by a group independently selected from —O—, —NH—, —N(C₁₋₅ alkyl)-, and —CO—. The C₁₋₅ alkylene is preferably a linear C₁₋₅ alkylene (e.g., a linear C₃₋₅ alkylene), more preferably a group —(CH₂)₁₋₅— (e.g., —(CH₂)₃₋₅—).

It is particularly preferred that each R^{A2} is independently selected from C₁₋₅ alkyl, C₂₋₅ alkenyl, C₂₋₅ alkynyl, halogen, C₁₋₅ haloalkyl, —(C₀₋₅ alkylene)-O(C₁₋₅ haloalkyl), —(C₀₋₅ alkylene)-CN, —(C₀₋₅ alkylene)-OH, —(C₀₋₅ alkylene)-O(C₁₋₅ alkyl), —(C₀₋₅ alkylene)-O(C₁₋₅ alkylene)-OH, —(C₀₋₅ alkylene)-O(C₁₋₅ alkylene)-O(C₁₋₅ alkyl), —(C₀₋₅ alkylene)-NH₂, —(C₀₋₅ alkylene)-NH(C₁₋₅ alkyl), —(C₀₋₅ alkylene)-N(C₁₋₅ alkyl)(C₁₋₅ alkyl), —(C₀₋₅ alkylene)-CHO, —(C₀₋₅ alkylene)-CO(C₁₋₅ alkyl), —(C₀₋₅ alkylene)-COOH, —(C₀₋₅ alkylene)-COO(C₁₋₅ alkyl), —(C₀₋₅ alkylene)-O—CO(C₁₋₅ alkyl), —(C₀₋₅ alkylene)-CO—NH₂, —(C₀₋₅ alkylene)-CO—NH(C₁₋₅ alkyl), —(C₀₋₅ alkylene)-CO—N(C₁₋₅ alkyl)(C₁₋₅ alkyl), —(C₀₋₅ alkylene)-NH—CO(C₁₋₅ alkyl), —(C₀₋₅ alkylene)-N(C₁₋₅ alkyl)-CO(C₁₋₅ alkyl), —(C₀₋₅ alkylene)-SH, —(C₀₋₅ alkylene)-S(C₁₋₅ alkyl), —(C₀₋₅ alkylene)-SO—(C₁₋₅ alkyl), —(C₀₋₅ alkylene)-SO₂—(C₁₋₅ alkyl), —(C₀₋₅ alkylene)-SO₂—NH₂, —(C₀₋₅ alkylene)-SO₂—NH(C₁₋₅ alkyl), —(C₀₋₅ alkylene)-SO₂—N(C₁₋₅ alkyl)(C₁₋₅ alkyl), —(C₀₋₅ alkylene)-NH—SO₂—(C₁₋₅ alkyl), —(C₀₋₅ alkylene)-N(C₁₋₅ alkyl)-SO₂—(C₁₋₅ alkyl), —(C₀₋₅ alkylene)-cycloalkyl, —(C₀₋₅ alkylene)-aryl, —(C₀₋₅ alkylene)-heterocycloalkyl, and —(C₀₋₅ alkylene)-heteroaryl, wherein the cycloalkyl moiety in said —(C₀₋₅ alkylene)-cycloalkyl, the aryl moiety in said —(C₀₋₅ alkylene)-aryl, the heterocycloalkyl moiety in said —(C₀₋₅ alkylene)-heterocycloalkyl, and the heteroaryl moiety in said —(C₀₋₅ alkylene)-heteroaryl are each optionally substituted with one or more (e.g., one, two or three) groups R^{Cyc}. Even more preferably, each R^{A2} is independently selected from C₁₋₅ alkyl, halogen, C₁₋₅ haloalkyl (e.g., —CF₃), —(C₀₋₃ alkylene)-O(C₁₋₅ haloalkyl) (e.g., —OCF₃), —(C₀₋₃ alkylene)-CN, —(C₀₋₃ alkylene)-OH, —(C₀₋₃ alkylene)-O(C₁₋₅ alkyl), —(C₀₋₃ alkylene)-NH₂, —(C₀₋₃ alkylene)-NH(C₁₋₅ alkyl), —(C₀₋₃ alkylene)-N(C₁₋₅ alkyl)(C₁₋₅ alkyl), —(C₀₋₃ alkylene)-cycloalkyl, —(C₀₋₃ alkylene)-aryl, —(C₀₋₃ alkylene)-heterocycloalkyl, and —(C₀₋₃ alkylene)-heteroaryl, wherein the cycloalkyl moiety in said —(C₀₋₃ alkylene)-cycloalkyl, the aryl moiety in said —(C₀₋₃ alkylene)-aryl, the heterocycloalkyl moiety in said —(C₀₋₃ alkylene)-heterocycloalkyl, and the heteroaryl moiety in said —(C₀₋₃ alkylene)-heteroaryl are each optionally substituted with one or more groups R^{Cyc}. Yet even more preferably, each R^{A2} is independently selected from C₁₋₅ alkyl (e.g., methyl, butyl or pentyl), —(C₀₋₃ alkylene)-cycloalkyl, —(C₀₋₃ alkylene)-aryl, —(C₀₋₃ alkylene)-heterocycloalkyl, and —(C₀₋₃ alkylene)-heteroaryl, wherein the cycloalkyl moiety in said —(C₀₋₃ alkylene)-cycloalkyl, the aryl moiety in said —(C₀₋₃ alkylene)-aryl, the heterocycloalkyl moiety in said —(C₀₋₃ alkylene)-heterocycloalkyl, and the heteroaryl moiety in said —(C₀₋₃ alkylene)-heteroaryl are each optionally substituted with one or more groups R^{Cyc}. Still more preferably, each R^{A2} is independently selected from C₁₋₅ alkyl, —(C₀₋₃ alkylene)-aryl and —(C₀₋₃ alkylene)-heteroaryl, wherein the aryl moiety in said —(C₀₋₃ alkylene)-aryl and the heteroaryl moiety in said —(C₀₋₃ alkylene)-heteroaryl are each optionally substituted with one or more groups R^{Cyc}. Specific preferred examples of R^{A2} include, in particular, methyl, n-butyl, cyclohexyl, —(C₀₋₃ alkylene)-phenyl (e.g., phenyl or benzyl), —(C₀₋₃ alkylene)-phenyl-halogen (e.g., 4-chlorophenyl or 4-chlorobenzyl), or —(C₀₋₃ alkylene)-imidazolyl (e.g., 3-(imidazol-5-yl)propyl).

It is preferred that ring A is substituted with one or more (e.g., one, two, three or four) groups R^{A2}. More preferably ring A is substituted with two or more groups R^{A2}. It is particularly preferred that ring A carries two substituents R^{A2} which are attached to the same ring carbon atom of ring A; in this case, the corresponding ring A may optionally be substituted with one or more further groups R^{A2}, i.e., it may either carry no further substituents R^{A2} or it may carry one or more (e.g., one or two) further groups R^{A2}, whereby it is preferred that the corresponding ring A carries no further substituents R^{A2}.

Thus, for example, if ring A is a group A1a, preferred examples of a corresponding group A1a which is substituted with one or more groups R^{A2} include, in particular, the following:

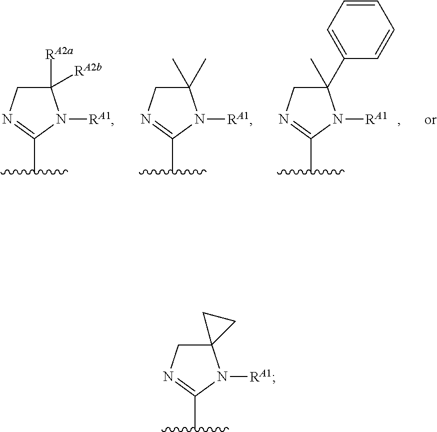

wherein each of the above-depicted groups is optionally further substituted with one or more groups R^{A2}; and wherein the groups R^{A2a} and R^{A2b} are each independently selected from C₁₋₅ alkyl, —(C₀₋₃ alkylene)-aryl and —(C₀₋₃ alkylene)-heteroaryl, wherein the aryl moiety in said —(C₀₋₃ alkylene)-aryl and the heteroaryl moiety in said —(C₀₋₃ alkylene)-heteroaryl are each optionally substituted with one or more groups R^{Cyc}, or wherein R^{A2a} and R^{A2b} are mutually joined to form, together with the ring carbon atom that they are attached to, a C₃₋₈ cycloalkyl which is optionally substituted with one or more groups R^{Cyc}.

Likewise, in the case that ring A is, for example, a group A2a1, preferred examples of a corresponding group A2a1 which is substituted with one or more groups R^{A2} include, in particular, the following:

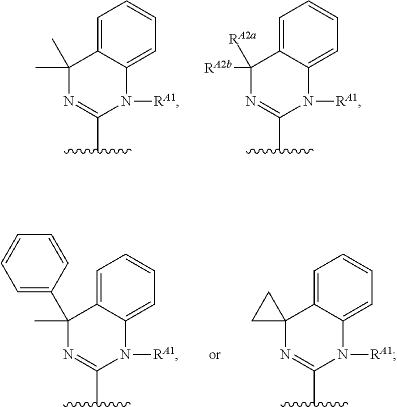

wherein each of the above-depicted groups is optionally further substituted with one or more groups R^{A2}; and wherein the groups R^{A2a} and R^{A2b} are each independently selected from C₁₋₅ alkyl, —(C₀₋₃ alkylene)-aryl and —(C₀₋₃ alkylene)-heteroaryl, wherein the aryl moiety in said —(C₀₋₃ alkylene)-aryl and the heteroaryl moiety in said —(C₀₋₃ alkylene)-heteroaryl are each optionally substituted with one or more groups R^{Cyc}, or wherein R^{A2a} and R^{A2b} are mutually joined to form, together with the ring carbon atom that they are attached to, a C₃₋₈ cycloalkyl which is optionally substituted with one or more groups R^{Cyc}.

Each R^{N} is independently selected from hydrogen, C₁₋₅ alkyl, C₂₋₅ alkenyl, C₂₋₅ alkynyl, —O(C₁₋₅ alkyl), —CO(C₁₋₅ alkyl), —COO(C₁₋₅ alkyl), carbocyclyl, and heterocyclyl, wherein said alkyl, said alkenyl, said alkynyl, the alkyl moiety in said —O(C₁₋₅ alkyl), the alkyl moiety in said —CO(C₁₋₅ alkyl), and the alkyl moiety in said —COO(C₁₋₅ alkyl) are each optionally substituted with one or more (e.g., one, two or three) groups R^{Alk}, wherein said carbocyclyl and said heterocyclyl are each optionally substituted with one or more (e.g., one, two or three) groups R^{Cyc}, and further wherein any two groups R^{N} which are attached to the same nitrogen atom may also be mutually joined to form, together with the nitrogen atom that they are attached to, a heterocyclyl which is optionally substituted with one or more (e.g., one, two or three) groups R^{Cyc}.

Preferably, each R^{N} is independently selected from hydrogen, C₁₋₅ alkyl, —O(C₁₋₅ alkyl), and —CO(C₁₋₅ alkyl), wherein said alkyl, the alkyl moiety in said —O(C₁₋₅ alkyl), and the alkyl moiety in said —CO(C₁₋₅ alkyl) are each optionally substituted with one or more groups R^{Alk}, and further wherein any two groups R^{N} which are attached to the same nitrogen atom may also be mutually joined to form, together with the nitrogen atom that they are attached to, a heterocyclyl which is optionally substituted with one or more groups R^{Cyc}. More preferably, each R^{N} is independently selected from hydrogen, C₁₋₅ alkyl, —O(C₁₋₅ alkyl), and —CO(C₁₋₅ alkyl), wherein said alkyl, the alkyl moiety in said —O(C₁₋₅ alkyl), and the alkyl moiety in said —CO(C₁₋₅ alkyl) are each optionally substituted with one or more groups R^{Alk}. Even more preferably, each R^{N} is independently selected from hydrogen, C₁₋₅ alkyl, —O(C₁₋₅ alkyl), and —CO(C₁₋₅ alkyl). Yet even more preferably, each R^{N} is independently selected from hydrogen and C₁₋₅ alkyl (e.g., methyl or ethyl).

Each R^{B1} is independently selected from C₁₋₅ alkyl, C₂₋₅ alkenyl, C₂₋₅ alkynyl, —(C₀₋₅ alkylene)-R^{B11}, —(C₂₋₅ alkenylene)-R^{B11}, —(C₂₋₅ alkynylene)-R^{B11}, and ═R^{B13}, wherein said alkyl, said alkenyl, said alkynyl, said alkylene, said alkenylene, and said alkynylene are each optionally substituted with one or more (e.g., one, two or three) groups R^{Alk}, and further wherein one or more (e.g., one, two or three) —CH₂— units comprised in said alkylene, said alkenylene, or said alkynylene are each optionally replaced by a group independently selected from —O—, —NH—, —N(C₁₋₅ alkyl)-, —CO—, —S—, —SO—, and —SO₂—;
wherein any two groups R^{B1}, which are attached to the same ring atom of ring B, may also be mutually joined to form, together with the ring atom that they are attached to, a cycloalkyl or a heterocycloalkyl, wherein said cycloalkyl or said heterocycloalkyl is optionally substituted with one or more (e.g., one, two or three) groups R^{Cyc}; and
wherein any two groups R^{B1}, which are attached to distinct ring atoms of ring B, may also be mutually joined to form a C₁₋₅ alkylene which is optionally substituted with one or more (e.g., one, two or three) groups R^{Cyc}, and wherein one or more (e.g., one, two or three) —CH₂— units comprised in said alkylene are each optionally replaced by a group independently selected from —O—, —NH—, —N(C₁₋₅ alkyl)-, —CO—, —S—, —SO—, and —SO₂—.

It will be understood that an optional substituent R^{B1}, if present, may be attached to any carbon ring atom or any nitrogen ring atom of the corresponding ring B, which carbon or nitrogen ring atom would otherwise (i.e., without R^{B1}) carry a hydrogen atom. Likewise, if two groups R^{B1} (which are attached to the same ring atom of ring B) are mutually joined to form a cycloalkyl or heterocycloalkyl (as described above), these groups R^{B1} may be attached to any carbon ring atom of ring B which would otherwise (i.e., without the two groups R^{B1}) carry two hydrogen atoms. Moreover, if two groups R^{B1} (which are attached to different ring atoms of ring B) are mutually joined to form a C₁₋₅ alkylene (as described above), these groups R^{B1} may be attached to any carbon ring atom or any nitrogen ring atom of the corresponding ring B, which carbon or nitrogen ring atom would otherwise (i.e., without R^{B1}) carry a hydrogen atom.

Each R^{B11} is independently selected from halogen, C₁₋₅ haloalkyl, —O(C₁₋₅ haloalkyl), —CN, —OR^{B12}, —NR^{B12}R^{B12}, —N⁺R^{B12}R^{B12}R^{B12}, —NR^{B12}OR^{B12}, —COR^{B12}, —COOR^{B12}, —OCOR^{B12}, —CONR^{B12}R^{B12}, —NR^{B12}COR^{B12}, —NR^{B12}COOR^{B12}, —OCONR^{B12}R^{B12}, —SR^{B12}, —SOR^{B12}, —SO₂R^{B12}, —SO₂NR^{B12}R^{B12}, —NR^{B12} SO₂R^{B12}, —SO₃R^{B12}, —NO₂, carbocyclyl, and heterocyclyl, wherein said carbocyclyl and said heterocyclyl are each optionally substituted with one or more (e.g., one, two or three) groups R^{Cyc}.

Preferably, each R^{B11} is independently selected from halogen, C₁₋₅ haloalkyl, —O(C₁₋₅ haloalkyl), —CN, —OR^{B12}, —NR^{B12}R^{B12}, —N⁺R^{B12}R^{B12}R^{B12}, —COR^{B12}, —COOR^{B12}, —OCOR^{B12}, —CONR^{B12}R^{B12}, —NR^{B12}COR^{B12}, —SR^{B12}, —SOR^{B12}, —SO₂R^{B12}, —SO₂NR^{B12}R^{B12}, —NR^{B12}SO₂R^{B12}, cycloalkyl, aryl, heterocycloalkyl, and heteroaryl, wherein said cycloalkyl, said aryl, said heterocycloalkyl and said heteroaryl are each optionally substituted with one or more (e.g., one, two or three) groups R^{Cyc}. More preferably, each R^{B11} is independently selected from halogen, C₁₋₅ haloalkyl, —O(C₁₋₅ haloalkyl), —CN, —OH, —O(C₁₋₅ alkyl), —O(C₁₋₅ alkylene)-OH, —O(C₁₋₅ alkylene)-O(C₁₋₅ alkyl), —NH₂, —NH(C₁₋₅ alkyl), —N(C₁₋₅ alkyl)(C₁₋₅ alkyl), —CHO, —CO(C₁₋₅ alkyl), —COOH, —COO(C₁₋₅ alkyl), —O—CO(C₁₋₅ alkyl), —CO—NH₂, —CO—NH(C₁₋₅ alkyl), —CO—N(C₁₋₅ alkyl)(C₁₋₅ alkyl), —NH—CO(C₁₋₅ alkyl), —N(C₁₋₅ alkyl)-CO(C₁₋₅ alkyl), —SH, —S(C₁₋₅ alkyl), —SO—(C₁₋₅ alkyl), —SO₂—(C₁₋₅ alkyl), —SO₂—NH₂, —SO₂—NH(C₁₋₅ alkyl), —SO₂—N(C₁₋₅ alkyl)(C_{1- 5} alkyl), —NH—SO₂—(C₁₋₅ alkyl), —N(C₁₋₅ alkyl)-SO₂—(C₁₋₅ alkyl), cycloalkyl, aryl, heterocycloalkyl, and heteroaryl, wherein said cycloalkyl, said aryl, said heterocycloalkyl and said heteroaryl are each optionally substituted with one or more groups R^{Cyc}. Even more preferably, each R^{B11} is independently selected from halogen, C₁₋₅ haloalkyl (e.g., —CF₃), —O(C₁₋₅ haloalkyl), —CN, —OH, —O(C₁₋₅ alkyl), —NH₂, —NH(C₁₋₅ alkyl), —N(C₁₋₅ alkyl)(C₁₋₅ alkyl), cycloalkyl, aryl, heterocycloalkyl, and heteroaryl, wherein said cycloalkyl, said aryl, said heterocycloalkyl and said heteroaryl are each optionally substituted with one or more groups R^{Cyc}.

Each R^{B12} is independently selected from hydrogen, C₁₋₅ alkyl, C₂₋₅ alkenyl, C₂₋₅ alkynyl, carbocyclyl, and heterocyclyl, wherein said alkyl, said alkenyl and said alkynyl are each optionally substituted with one or more (e.g., one, two or three) groups R^{Alk}, and further wherein said carbocyclyl and said heterocyclyl are each optionally substituted with one or more (e.g., one, two or three) groups R^{Cyc}.

Preferably, each R^{B12} is independently selected from hydrogen and C₁₋₅ alkyl, wherein said alkyl is optionally substituted with one or more groups R^{Alk}. More preferably, each R^{B12} is independently selected from hydrogen and C₁₋₅ alkyl (e.g., methyl or ethyl).

Each R^{B13} is independently selected from ═O, ═S, and ═N—R^{B12}. Preferably, R^{B13} is ═O.

As explained above, each R^{B1} may be independently selected from C₁₋₅ alkyl, C₂₋₅ alkenyl, C₂₋₅ alkynyl, —(C₀₋₅ alkylene)-R^{B11}, —(C₂₋₅ alkenylene)-R^{B11}, —(C₂₋₅ alkynylene)-R^{B11}, and ═R^{B13}, wherein said alkyl, said alkenyl, said alkynyl, said alkylene, said alkenylene, and said alkynylene are each optionally substituted with one or more groups R^{Alk}, and further wherein one or more —CH₂— units comprised in said alkylene, said alkenylene, or said alkynylene are each optionally replaced by a group independently selected from —O—, —NH—, —N(C₁₋₅ alkyl)-, —CO—, —S—, —SO—, and —SO₂—. In this case, and in accordance with the above definition of R^{B11}, it is preferred that each R^{B1} is independently selected from C₁₋₅ alkyl, C₂₋₅ alkenyl, C₂₋₅ alkynyl, halogen, C₁₋₅ haloalkyl, —(C₀₋₅ alkylene)-O(C₁₋₅ haloalkyl), —(C₀₋₅ alkylene)-CN, —(C₀₋₅ alkylene)-OH, —(C₀₋₅ alkylene)-O(C₁₋₅ alkyl), —(C₀₋₅ alkylene)-O(C₁₋₅ alkylene)-OH, —(C₀₋₅ alkylene)-O(C₁₋₅ alkylene)-O(C₁₋₅ alkyl), —(C₀₋₅ alkylene)-NH₂, —(C₀₋₅ alkylene)-NH(C₁₋₅ alkyl), —(C₀₋₅ alkylene)-N(C₁₋₅ alkyl)(C₁₋₅ alkyl), —(C₀₋₅ alkylene)-CHO, —(C₀₋₅ alkylene)-CO(C₁₋₅ alkyl), —(C₀₋₅ alkylene)-COOH, —(C₀₋₅ alkylene)-COO(C₁₋₅ alkyl), —(C₀₋₅ alkylene)-O—CO(C₁₋₅ alkyl), —(C₀₋₅ alkylene)-CO—NH₂, —(C₀₋₅ alkylene)-CO—NH(C₁₋₅ alkyl), —(C₀₋₅ alkylene)-CO—N(C₁₋₅ alkyl)(C₁₋₅ alkyl), —(C₀₋₅ alkylene)-NH—CO(C₁₋₅ alkyl), —(C₀₋₅ alkylene)-N(C₁₋₅ alkyl)-CO(C₁₋₅ alkyl), —(C₀₋₅ alkylene)-SH, —(C₀₋₅ alkylene)-S(C₁₋₅ alkyl), —(C₀₋₅ alkylene)-SO—(C₁₋₅ alkyl), —(C₀₋₅ alkylene)-SO₂—(C₁₋₅ alkyl), —(C₀₋₅ alkylene)-SO₂—NH₂, —(C₀₋₅ alkylene)-SO₂—NH(C₁₋₅ alkyl), —(C₀₋₅ alkylene)-SO₂—N(C₁₋₅ alkyl)(C₁₋₅ alkyl), —(C₀₋₅ alkylene)-NH—SO₂—(C₁₋₅ alkyl), —(C₀₋₅ alkylene)-N(C₁₋₅ alkyl)-SO₂—(C₁₋₅ alkyl), —(C₀₋₅ alkylene)-cycloalkyl, —(C₀₋₅ alkylene)-aryl, —(C₀₋₅ alkylene)-heterocycloalkyl, and —(C₀₋₅ alkylene)-heteroaryl, wherein the cycloalkyl moiety in said —(C₀₋₅ alkylene)-cycloalkyl, the aryl moiety in said —(C₀₋₅ alkylene)-aryl, the heterocycloalkyl moiety in said —(C₀₋₅ alkylene)-heterocycloalkyl, and the heteroaryl moiety in said —(C₀₋₅ alkylene)-heteroaryl are each optionally substituted with one or more (e.g., one, two or three) groups R^{Cyc}. Moreover, R^{B1} may also be ═O. More preferably, each R^{B1} is independently selected from C₁₋₅ alkyl, halogen, C₁₋₅ haloalkyl (e.g., —CF₃), —(C₀₋₃ alkylene)-O(C₁₋₅ haloalkyl) (e.g., —OCF₃), —(C₀₋₃ alkylene)-CN, —(C₀₋₃ alkylene)-OH, —(C₀₋₃ alkylene)-O(C₁₋₅ alkyl), —(C₀₋₃ alkylene)-NH₂, —(C₀₋₃ alkylene)-NH(C₁₋₅ alkyl), —(C₀₋₃ alkylene)-N(C₁₋₅ alkyl)(C₁₋₅ alkyl), —(C₀₋₃ alkylene)-cycloalkyl, —(C₀₋₃ alkylene)-aryl, —(C₀₋₃ alkylene)-heterocycloalkyl, and —(C₀₋₃ alkylene)-heteroaryl, wherein the cycloalkyl moiety in said —(C₀₋₃ alkylene)-cycloalkyl, the aryl moiety in said —(C₀₋₃ alkylene)-aryl, the heterocycloalkyl moiety in said —(C₀₋₃ alkylene)-heterocycloalkyl, and the heteroaryl moiety in said —(C₀₋₃ alkylene)-heteroaryl are each optionally substituted with one or more groups R^{Cyc}.

As also explained above, any two groups R^{B1}, which are attached to the same ring atom (particularly the same carbon ring atom) of ring B, may be mutually joined to form, together with the ring atom that they are attached to, a cycloalkyl or a heterocycloalkyl (which is optionally substituted with one or more groups R^{Cyc}). It is preferred that the cycloalkyl or heterocycloalkyl which is formed from such two groups R^{B1}, and which is optionally substituted with one or more groups R^{Cyc}, has 3 to 14 ring members, more preferably 3 to 10 (i.e., 3, 4, 5, 6, 7, 8, 9 or 10) ring members. Moreover, it is preferred that said cycloalkyl or said heterocycloalkyl is monocyclic, bridged polycyclic (e.g., bridged bicyclic), or fused polycyclic (e.g., fused bicyclic); more preferably, said cycloalkyl or said heterocycloalkyl is monocyclic or bridged bicyclic. It is particularly preferred that the cycloalkyl which is formed from two groups R^{B1}, and which is optionally substituted with one or more groups R^{Cyc}, is a monocyclic C₃₋₇ cycloalkyl (e.g., cyclopropyl, cyclobutyl, cyclopentyl, cyclohexyl, or cycloheptyl) or a bicyclic bridged C₇₋₁₀ cycloalkyl (e.g., norbornanyl or adamantyl). It is furthermore particularly preferred that the heterocycloalkyl which is formed from two groups R^{B1}, and which is optionally substituted with one or more groups R^{Cyc}, is a monocyclic 3 to 7-membered heterocycloalkyl (e.g., azetidinyl, oxetanyl, thietanyl, pyrrolidinyl, tetrahydrofuranyl, tetrahydrothiophenyl, piperidinyl, tetrahydropyranyl, or thianyl) or a bicyclic bridged 7 to 10-membered heterocycloalkyl (e.g., quinuclidinyl or nortropanyl). Unless defined otherwise, it is preferred that no groups R^{B1}, which are attached to the same ring atom of ring B, are mutually joined.

As also explained above, any two groups R^{B1}, which are attached to distinct (i.e., different) ring atoms of ring B, may be mutually joined to form a C₁₋₅ alkylene, wherein said alkylene is optionally substituted with one or more groups R^{Cyc}, and wherein one or more —CH₂— units comprised in said alkylene are each optionally replaced by a group independently selected from —O—, —NH—, —N(C₁₋₅ alkyl)-, —CO—, —S—, —SO—, and —SO₂—. It is preferred that said alkylene is optionally substituted with one or two groups R^{Cyc}, and it is furthermore preferred that one or two —CH₂— units comprised in said alkylene are each optionally replaced by a group independently selected from —O—, —NH—, —N(C₁₋₅ alkyl)-, and —CO—. Moreover, said C₁₋₅ alkylene is preferably selected from —CH₂—, —CH₂CH₂—, —CH₂CH₂CH₂—, —CH₂CH₂CH₂CH₂— and —CH₂CH₂CH₂CH₂CH₂—. If two groups R^{B1}, which are attached to non-adjacent ring atoms of ring B (e.g., there may be at least one other ring atom (preferably one, two or three other ring atoms) in between these two ring atoms of ring B), are mutually joined to form an alkylene (which is optionally substituted with one or more R^{Cyc}, and wherein one or more —CH₂— units comprised in the alkylene are each optionally replaced, as defined above), it is preferred that said alkylene is a C₁₋₃ alkylene, more preferably —CH₂—, —CH₂CH₂— or —CH₂CH₂CH₂—. If two groups R^{B1}, which are attached to adjacent ring atoms of ring B, are mutually joined to form an alkylene (which is optionally substituted with one or more R^{Cyc}, and wherein one or more —CH₂— units comprised in the alkylene are each optionally replaced, as defined above), it is preferred that said alkylene is a C₃₋₅ alkylene, more preferably —CH₂CH₂CH₂—, —CH₂CH₂CH₂CH₂— or —CH₂CH₂CH₂CH₂CH₂—. Unless defined otherwise, it is preferred that no groups R^{B1}, which are attached to distinct ring atoms of ring B, are mutually joined.

It is particularly preferred that each R^{B1} is independently selected from C₁₋₅ alkyl, C₂₋₅ alkenyl, C₂₋₅ alkynyl, halogen, C₁₋₅ haloalkyl, —(C₀₋₅ alkylene)-O(C₁₋₅ haloalkyl), —(C₀₋₅ alkylene)-CN, —(C₀₋₅ alkylene)-OH, —(C₀₋₅ alkylene)-O(C₁₋₅ alkyl), —(C₀₋₅ alkylene)-O(C₁₋₅ alkylene)-OH, —(C₀₋₅ alkylene)-O(C₁₋₅ alkylene)-O(C₁₋₅ alkyl), —(C₀₋₅ alkylene)-NH₂, —(C₀₋₅ alkylene)-NH(C₁₋₅ alkyl), —(C₀₋₅ alkylene)-N(C₁₋₅ alkyl)(C₁₋₅ alkyl), —(C₀₋₅ alkylene)-CHO, —(C₀₋₅ alkylene)-CO(C₁₋₅ alkyl), —(C₀₋₅ alkylene)-COOH, —(C₀₋₅ alkylene)-COO(C₁₋₅ alkyl), —(C₀₋₅ alkylene)-O—CO(C₁₋₅ alkyl), —(C₀₋₅ alkylene)-CO—NH₂, —(C₀₋₅ alkylene)-CO—NH(C₁₋₅ alkyl), —(C₀₋₅ alkylene)-CO—N(C₁₋₅ alkyl)(C₁₋₅ alkyl), —(C₀₋₅ alkylene)-NH—CO(C₁₋₅ alkyl), —(C₀₋₅ alkylene)-N(C₁₋₅ alkyl)-CO(C₁₋₅ alkyl), —(C₀₋₅ alkylene)-SH, —(C₀₋₅ alkylene)-S(C₁₋₅ alkyl), —(C₀₋₅ alkylene)-SO—(C₁₋₅ alkyl), —(C₀₋₅ alkylene)-SO₂—(C₁₋₅ alkyl), —(C₀₋₅ alkylene)-SO₂—NH₂, —(C₀₋₅ alkylene)-SO₂—NH(C₁₋₅ alkyl), —(C₀₋₅ alkylene)-SO₂—N(C₁₋₅ alkyl)(C₁₋₅ alkyl), —(C₀₋₅ alkylene)-NH—SO₂—(C₁₋₅ alkyl), —(C₀₋₅ alkylene)-N(C₁₋₅ alkyl)-SO₂—(C₁₋₅ alkyl), —(C₀₋₅ alkylene)-cycloalkyl, —(C₀₋₅ alkylene)-aryl (e.g., phenyl or benzyl), —(C₀₋₅ alkylene)-heterocycloalkyl, and —(C₀₋₅ alkylene)-heteroaryl, wherein the cycloalkyl moiety in said —(C₀₋₅ alkylene)-cycloalkyl, the aryl moiety in said —(C₀₋₅ alkylene)-aryl, the heterocycloalkyl moiety in said —(C₀₋₅ alkylene)-heterocycloalkyl, and the heteroaryl moiety in said —(C₀₋₅ alkylene)-heteroaryl are each optionally substituted with one or more (e.g., one, two or three) groups R^{Cyc}. Even more preferably, each R^{B1} is independently selected from C₁₋₅ alkyl, halogen, C₁₋₅ haloalkyl (e.g., —CF₃), —(C₀₋₃ alkylene)-O(C₁₋₅ haloalkyl) (e.g., —OCF₃), —(C₀₋₃ alkylene)-CN, —(C₀₋₃ alkylene)-OH, —(C₀₋₃ alkylene)-O(C₁₋₅ alkyl), —(C₀₋₃ alkylene)-NH₂, —(C₀₋₃ alkylene)-NH(C₁₋₅ alkyl), —(C₀₋₃ alkylene)-N(C₁₋₅ alkyl)(C₁₋₅ alkyl), —(C₀₋₃ alkylene)-cycloalkyl, —(C₀₋₃ alkylene)-aryl, —(C₀₋₃ alkylene)-heterocycloalkyl, and —(C₀₋₃ alkylene)-heteroaryl, wherein the cycloalkyl moiety in said —(C₀₋₃ alkylene)-cycloalkyl, the aryl moiety in said —(C₀₋₃ alkylene)-aryl, the heterocycloalkyl moiety in said —(C₀₋₃ alkylene)-heterocycloalkyl, and the heteroaryl moiety in said —(C₀₋₃ alkylene)-heteroaryl are each optionally substituted with one or more groups R^{Cyc}.

For example, if ring B is a tricyclic ring group substituted with one or more groups R^{B1}, it is preferred that at least one substituent R^{B1} is a halogen (e.g., —F or —Cl), a C₁₋₅ haloalkyl (e.g., —CF₃), a —(C₀₋₃ alkylene)-O(C₁₋₅ haloalkyl) (e.g., —OCF₃), or a —(C₀₋₃ alkylene)-CN (e.g., —CN), which is attached to the most distant ring comprised in the tricyclic ring group (as viewed from the attachment point of ring B to the group L), e.g., as illustrated by the following exemplary ring B groups:

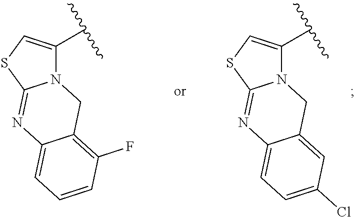 [SMILES: *C1=CSC2=Nc3cccc(F)c3CN12 ; *C1=CSC2=Nc3ccc(Cl)cc3CN12]

wherein each of the above-depicted groups is optionally further substituted with one or more groups R^{B1}.

Moreover, if a group R^{B1} is attached to a carbon ring atom of ring B, which carbon ring atom is adjacent to the ring atom through which ring B is attached to the group L, then this group R^{B1} may be, in particular, C₁₋₅ alkyl (e.g., methyl, ethyl, or isopropyl), cycloalkyl (e.g., cyclopropyl), or halogen (e.g., —I).

Each R^{B2} is independently selected from C₁₋₅ alkyl, C₂₋₅ alkenyl, C₂₋₅ alkynyl, —(C₀₋₅ alkylene)-R^{B21}, —(C₂₋₅ alkenylene)-R^{B21}, and —(C₂₋₅ alkynylene)-R^{B21}, wherein said alkyl, said alkenyl, said alkynyl, said alkylene, said alkenylene, and said alkynylene are each optionally substituted with one or more (e.g., one, two or three) groups R^{Alk}, and further wherein one or more (e.g., one, two or three) —CH₂— units comprised in said alkylene, said alkenylene, or said alkynylene are each optionally replaced by a group independently selected from —O—, —NH—, —N(C₁₋₅ alkyl)-, —CO—, —S—, —SO—, and —SO₂—.

Each R^{B21} is independently selected from halogen, C₁₋₅ haloalkyl, —O(C₁₋₅ haloalkyl), —CN, —OR^{B12}, —COR^{B12}, —COOR^{B12}, —OCOR^{B12}, —CONR^{B12}R^{B12}, —OCONR^{B12}R^{B12}, —SR^{B12}, —SOR^{B12}, —SO₂R^{B12}, —SO₂NR^{B12}R^{B12}, —NR^{B12}SO₂R^{B12}, —SO₃R^{B12}, —NO₂, carbocyclyl, and heterocyclyl, wherein said carbocyclyl and said heterocyclyl are each optionally substituted with one or more (e.g., one, two or three) groups R^{Cyc}.

Preferably, each R^{B21} is independently selected from halogen, C₁₋₅ haloalkyl, —O(C₁₋₅ haloalkyl), —CN, —OR^{B12}, —COR^{B12}, —COOR^{B12}, —OCOR^{B12}, —CONR^{B12}R^{B12}, —SR^{B12}, —SOR^{B12}, —SO₂R^{B12}, —SO₂NR^{B12}R^{B12}, —NR^{B12}SO₂R^{B12}, cycloalkyl, aryl, heterocycloalkyl, and heteroaryl, wherein said cycloalkyl, said aryl, said heterocycloalkyl and said heteroaryl are each optionally substituted with one or more (e.g., one, two or three) groups R^{Cyc}. More preferably, each R^{B21} is independently selected from halogen, C₁₋₅ haloalkyl, —O(C₁₋₅ haloalkyl), —CN, —OH, —O(C₁₋₅ alkyl), —O(C₁₋₅ alkylene)-OH, —O(C₁₋₅ alkylene)-O(C₁₋₅ alkyl), —CHO, —CO(C₁₋₅ alkyl), —COOH, —COO(C₁₋₅ alkyl), —O—CO(C₁₋₅ alkyl), —CO—NH₂, —CO—NH(C₁₋₅ alkyl), —CO—N(C₁₋₅ alkyl)(C₁₋₅ alkyl), —SH, —S(C₁₋₅ alkyl), —SO—(C₁₋₅ alkyl), —SO₂—(C₁₋₅ alkyl), —SO₂—NH₂, —SO₂—NH(C₁₋₅ alkyl), —SO₂—N(C₁₋₅ alkyl)(C_{1- 5} alkyl), —NH—SO₂—(C₁₋₅ alkyl), —N(C₁₋₅ alkyl)-SO₂—(C₁₋₅ alkyl), cycloalkyl, aryl, heterocycloalkyl, and heteroaryl, wherein said cycloalkyl, said aryl, said heterocycloalkyl and said heteroaryl are each optionally substituted with one or more groups R^{Cyc}. Even more preferably, each R^{B21} is independently selected from halogen, C₁₋₅ haloalkyl (e.g., —CF₃), —O(C₁₋₅ haloalkyl), —CN, —OH, —O(C₁₋₅ alkyl), cycloalkyl, aryl, heterocycloalkyl, and heteroaryl, wherein said cycloalkyl, said aryl, said heterocycloalkyl and said heteroaryl are each optionally substituted with one or more groups R^{Cyc}.

Thus, in accordance with the above definition of R^{B21}, it is particularly preferred that each R^{B2} is independently selected from C₁₋₅ alkyl, C₂₋₅ alkenyl, C₂₋₅ alkynyl, halogen, C₁₋₅ haloalkyl, —(C₀₋₅ alkylene)-O(C₁₋₅ haloalkyl), —(C₀₋₅ alkylene)-CN, —(C₀₋₅ alkylene)-OH, —(C₀₋₅ alkylene)-O(C₁₋₅ alkyl), —(C₀₋₅ alkylene)-O(C₁₋₅ alkylene)-OH, —(C₀₋₅ alkylene)-O(C₁₋₅ alkylene)-O(C₁₋₅ alkyl), —(C₀₋₅ alkylene)-CHO, —(C₀₋₅ alkylene)-CO(C₁₋₅ alkyl), —(C₀₋₅ alkylene)-COOH, —(C₀₋₅ alkylene)-COO(C₁₋₅ alkyl), —(C₀₋₅ alkylene)-O—CO(C₁₋₅ alkyl), —(C₀₋₅ alkylene)-CO—NH₂, —(C₀₋₅ alkylene)-CO—NH(C₁₋₅ alkyl), —(C₀₋₅ alkylene)-CO—N(C₁₋₅ alkyl)(C₁₋₅ alkyl), —(C₀₋₅ alkylene)-SH, —(C₀₋₅ alkylene)-S(C₁₋₅ alkyl), —(C₀₋₅ alkylene)-SO—(C₁₋₅ alkyl), —(C₀₋₅ alkylene)-SO₂—(C₁₋₅ alkyl), —(C₀₋₅ alkylene)-SO₂—NH₂, —(C₀₋₅ alkylene)-SO₂—NH(C₁₋₅ alkyl), —(C₀₋₅ alkylene)-SO₂—N(C₁₋₅ alkyl)(C₁₋₅ alkyl), —(C₀₋₅ alkylene)-NH—SO₂—(C₁₋₅ alkyl), —(C₀₋₅ alkylene)-N(C₁₋₅ alkyl)-SO₂—(C₁₋₅ alkyl), —(C₀₋₅ alkylene)-cycloalkyl, —(C₀₋₅ alkylene)-aryl, —(C₀₋₅ alkylene)-heterocycloalkyl, and —(C₀₋₅ alkylene)-heteroaryl, wherein the cycloalkyl moiety in said —(C₀₋₅ alkylene)-cycloalkyl, the aryl moiety in said —(C₀₋₅ alkylene)-aryl, the heterocycloalkyl moiety in said —(C₀₋₅ alkylene)-heterocycloalkyl, and the heteroaryl moiety in said —(C₀₋₅ alkylene)-heteroaryl are each optionally substituted with one or more (e.g., one, two or three) groups R^{Cyc}. More preferably, each R^{B2} is independently selected from C₁₋₅ alkyl, halogen, C₁₋₅ haloalkyl (e.g., —CF₃), —(C₀₋₃ alkylene)-O(C₁₋₅ haloalkyl) (e.g., —OCF₃), —(C₀₋₃ alkylene)-CN, —(C₀₋₃ alkylene)-OH, —(C₀₋₃ alkylene)-O(C₁₋₅ alkyl), —(C₀₋₃ alkylene)-cycloalkyl, —(C₀₋₃ alkylene)-aryl, —(C₀₋₃ alkylene)-heterocycloalkyl, and —(C₀₋₃ alkylene)-heteroaryl, wherein the cycloalkyl moiety in said —(C₀₋₃ alkylene)-cycloalkyl, the aryl moiety in said —(C₀₋₃ alkylene)-aryl, the heterocycloalkyl moiety in said —(C₀₋₃ alkylene)-heterocycloalkyl, and the heteroaryl moiety in said —(C₀₋₃ alkylene)-heteroaryl are each optionally substituted with one or more groups R^{Cyc}.

Each R^{Alk} is independently selected from —OH, —O(C₁₋₅ alkyl), —O(C₁₋₅ alkylene)-OH, —O(C₁₋₅ alkylene)-O(C₁₋₅ alkyl), —SH, —S(C₁₋₅ alkyl), —S(C₁₋₅ alkylene)-SH, —S(C₁₋₅ alkylene)-S(C₁₋₅ alkyl), —NH₂, —NH(C₁₋₅ alkyl), —N(C₁₋₅ alkyl)(C₁₋₅ alkyl), —NH—OH, —N(C₁₋₅ alkyl)-OH, —NH—O(C₁₋₅ alkyl), —N(C₁₋₅ alkyl)-O(C₁₋₅ alkyl), halogen, C₁₋₅ haloalkyl, —O(C₁₋₅ haloalkyl), —CN, —NO₂, —CHO, —CO(C₁₋₅ alkyl), —COOH, —COO(C₁₋₅ alkyl), —O—CO(C₁₋₅ alkyl), —CO—NH₂, —CO—NH(C₁₋₅ alkyl), —CO—N(C₁₋₅ alkyl)(C₁₋₅ alkyl), —NH—CO(C₁₋₅ alkyl), —N(C₁₋₅ alkyl)-CO(C₁₋₅ alkyl), —NH—COO(C₁₋₅ alkyl), —N(C₁₋₅ alkyl)-COO(C₁₋₅ alkyl), —O—CO—NH(C₁₋₅ alkyl), —O—CO—N(C₁₋₅ alkyl)(C₁₋₅ alkyl), —SO₂—NH₂, —SO₂—NH(C₁₋₅ alkyl), —SO₂—N(C₁₋₅ alkyl)(C₁₋₅ alkyl), —NH—SO₂—(C₁₋₅ alkyl), —N(C₁₋₅ alkyl)-SO₂—(C₁₋₅ alkyl), —SO₂—(C₁₋₅ alkyl), —SO—(C_{1- 5} alkyl), aryl, heteroaryl, cycloalkyl, heterocycloalkyl, and -L^{X}-R^{X}, wherein said aryl, said heteroaryl, said cycloalkyl, and said heterocycloalkyl are each optionally substituted with one or more (e.g., one, two or three) groups independently selected from C₁₋₅ alkyl, C₂₋₅ alkenyl, C₂₋₅ alkynyl, halogen, C₁₋₅ haloalkyl, —O(C₁₋₅ haloalkyl), —CN, —OH, —O(C₁₋₅ alkyl), —SH, —S(C₁₋₅ alkyl), —NH₂, —NH(C₁₋₅ alkyl), and —N(C₁₋₅ alkyl)(C₁₋₅ alkyl).

Preferably, each R^{Alk} is independently selected from —OH, —O(C₁₋₅ alkyl), —O(C₁₋₅ alkylene)-OH, —O(C₁₋₅ alkylene)-O(C₁₋₅ alkyl), —SH, —S(C₁₋₅ alkyl), —NH₂, —NH(C₁₋₅ alkyl), —N(C₁₋₅ alkyl)(C₁₋₅ alkyl), halogen, C₁₋₅ haloalkyl, —O(C₁₋₅ haloalkyl), —CN, —CHO, —CO(C₁₋₅ alkyl), —COOH, —COO(C₁₋₅ alkyl), —O—CO(C₁₋₅ alkyl), —CO—NH₂, —CO—NH(C₁₋₅ alkyl), —CO—N(C₁₋₅ alkyl)(C₁₋₅ alkyl), —NH—CO(C₁₋₅ alkyl), —N(C₁₋₅ alkyl)-CO(C₁₋₅ alkyl), —NH—COO(C₁₋₅ alkyl), —N(C₁₋₅ alkyl)-COO(C₁₋₅ alkyl), —O—CO—NH(C₁₋₅ alkyl), —O—CO—N(C₁₋₅ alkyl)(C₁₋₅ alkyl), —SO₂—NH₂, —SO₂—NH(C₁₋₅ alkyl), —SO₂—N(C₁₋₅ alkyl)(C₁₋₅ alkyl), —NH—SO₂—(C₁₋₅ alkyl), —N(C₁₋₅ alkyl)-SO₂—(C₁₋₅ alkyl), —SO₂—(C₁₋₅ alkyl), —SO—(C_{1- 5} alkyl), aryl, heteroaryl, cycloalkyl, and heterocycloalkyl, wherein said aryl, said heteroaryl, said cycloalkyl, and said heterocycloalkyl are each optionally substituted with one or more groups independently selected from C₁₋₅ alkyl, C₂₋₅ alkenyl, C₂₋₅ alkynyl, halogen, C₁₋₅ haloalkyl, —O(C₁₋₅ haloalkyl), —CN, —OH, —O(C₁₋₅ alkyl), —SH, —S(C₁₋₅ alkyl), —NH₂, —NH(C₁₋₅ alkyl), and —N(C₁₋₅ alkyl)(C₁₋₅ alkyl). More preferably, each R^{Alk} is independently selected from —OH, —O(C₁₋₅ alkyl), —NH₂, —NH(C₁₋₅ alkyl), —N(C₁₋₅ alkyl)(C₁₋₅ alkyl), halogen, C₁₋₅ haloalkyl, —O(C₁₋₅ haloalkyl), and —CN.

Each R^{Cyc} is independently selected from C₁₋₅ alkyl, C₂₋₅ alkenyl, C₂₋₅ alkynyl, —OH, —O(C₁₋₅ alkyl), —O(C₁₋₅ alkylene)-OH, —O(C₁₋₅ alkylene)-O(C₁₋₅ alkyl), —SH, —S(C₁₋₅ alkyl), —S(C₁₋₅ alkylene)-SH, —S(C₁₋₅ alkylene)-S(C₁₋₅ alkyl), —NH₂, —NH(C₁₋₅ alkyl), —N(C₁₋₅ alkyl)(C₁₋₅ alkyl), —NH—OH, —N(C₁₋₅ alkyl)-OH, —NH—O(C₁₋₅ alkyl), —N(C₁₋₅ alkyl)-O(C₁₋₅ alkyl), halogen, C₁₋₅ haloalkyl, —O(C₁₋₅ haloalkyl), —CN, —NO₂, —CHO, —CO(C₁₋₅ alkyl), —COOH, —COO(C₁₋₅ alkyl), —O—CO(C₁₋₅ alkyl), —CO—NH₂, —CO—NH(C₁₋₅ alkyl), —CO—N(C₁₋₅ alkyl)(C₁₋₅ alkyl), —NH—CO(C₁₋₅ alkyl), —N(C₁₋₅ alkyl)-CO(C₁₋₅ alkyl), —NH—COO(C₁₋₅ alkyl), —N(C₁₋₅ alkyl)-COO(C₁₋₅ alkyl), —O—CO—NH(C₁₋₅ alkyl), —O—CO—N(C₁₋₅ alkyl)(C₁₋₅ alkyl), —SO₂—NH₂, —SO₂—NH(C₁₋₅ alkyl), —SO₂—N(C₁₋₅ alkyl)(C₁₋₅ alkyl), —NH—SO₂—(C₁₋₅ alkyl), —N(C₁₋₅ alkyl)-SO₂—(C₁₋₅ alkyl), —SO₂—(C₁₋₅ alkyl), —SO—(C₁₋₅ alkyl), aryl, heteroaryl, cycloalkyl, heterocycloalkyl, and -L^{X}-R^{X}, wherein said aryl, said heteroaryl, said cycloalkyl, and said heterocycloalkyl are each optionally substituted with one or more (e.g., one, two or three) groups independently selected from C₁₋₅ alkyl, C₂₋₅ alkenyl, C₂₋₅ alkynyl, halogen, C₁₋₅ haloalkyl, —O(C₁₋₅ haloalkyl), —CN, —OH, —O(C₁₋₅ alkyl), —SH, —S(C₁₋₅ alkyl), —NH₂, —NH(C₁₋₅ alkyl), and —N(C₁₋₅ alkyl)(C₁₋₅ alkyl).

Preferably, each R^{Cyc} is independently selected from C₁₋₅ alkyl, C₂₋₅ alkenyl, C₂₋₅ alkynyl, —OH, —O(C₁₋₅ alkyl), —O(C₁₋₅ alkylene)-OH, —O(C₁₋₅ alkylene)-O(C₁₋₅ alkyl), —SH, —S(C₁₋₅ alkyl), —NH₂, —NH(C₁₋₅ alkyl), —N(C₁₋₅ alkyl)(C₁₋₅ alkyl), halogen, C₁₋₅ haloalkyl, —O(C₁₋₅ haloalkyl), —CN, —CHO, —CO(C₁₋₅ alkyl), —COOH, —COO(C₁₋₅ alkyl), —O—CO(C₁₋₅ alkyl), —CO—NH₂, —CO—NH(C₁₋₅ alkyl), —CO—N(C₁₋₅ alkyl)(C₁₋₅ alkyl), —NH—CO(C₁₋₅ alkyl), —N(C₁₋₅ alkyl)-CO(C₁₋₅ alkyl), —NH—COO(C₁₋₅ alkyl), —N(C₁₋₅ alkyl)-COO(C₁₋₅ alkyl), —O—CO—NH(C₁₋₅ alkyl), —O—CO—N(C₁₋₅ alkyl)(C₁₋₅ alkyl), —SO₂—NH₂, —SO₂—NH(C₁₋₅ alkyl), —SO₂—N(C₁₋₅ alkyl)(C₁₋₅ alkyl), —NH—SO₂—(C₁₋₅ alkyl), —N(C₁₋₅ alkyl)-SO₂—(C₁₋₅ alkyl), —SO₂—(C₁₋₅ alkyl), —SO—(C₁₋₅ alkyl), aryl, heteroaryl, cycloalkyl, and heterocycloalkyl, wherein said aryl, said heteroaryl, said cycloalkyl, and said heterocycloalkyl are each optionally substituted with one or more groups independently selected from C₁₋₅ alkyl, C₂₋₅ alkenyl, C₂₋₅ alkynyl, halogen, C₁₋₅ haloalkyl, —O(C₁₋₅ haloalkyl), —CN, —OH, —O(C₁₋₅ alkyl), —SH, —S(C₁₋₅ alkyl), —NH₂, —NH(C₁₋₅ alkyl), and —N(C₁₋₅ alkyl)(C₁₋₅ alkyl). More preferably, each R^{Cyc} is independently selected from C₁₋₅ alkyl, —OH, —O(C₁₋₅ alkyl), —NH₂, —NH(C₁₋₅ alkyl), —N(C₁₋₅ alkyl)(C₁₋₅ alkyl), halogen, C₁₋₅ haloalkyl, —O(C₁₋₅ haloalkyl), and —CN.

Each L^{X} is independently selected from a bond, C₁₋₅ alkylene, C₂₋₅ alkenylene, and C₂₋₅ alkynylene, wherein said alkylene, said alkenylene and said alkynylene are each optionally substituted with one or more (e.g., one, two or three) groups independently selected from halogen, C₁₋₅ haloalkyl, —CN, —OH, —O(C₁₋₅ alkyl), —SH, —S(C₁₋₅ alkyl), —NH₂, —NH(C₁₋₅ alkyl), and —N(C₁₋₅ alkyl)(C₁₋₅ alkyl), and further wherein one or more (e.g., one, two or three) —CH₂— units comprised in said alkylene, said alkenylene or said alkynylene are each optionally replaced by a group independently selected from —O—, —NH—, —N(C₁₋₅ alkyl)-, —CO—, —S—, —SO—, and —SO₂—.

Each R^{X} is independently selected from —OH, —O(C₁₋₅ alkyl), —O(C₁₋₅ alkylene)-OH, —O(C₁₋₅ alkylene)-O(C₁₋₅ alkyl), —SH, —S(C₁₋₅ alkyl), —S(C₁₋₅ alkylene)-SH, —S(C₁₋₅ alkylene)-S(C₁₋₅ alkyl), —NH₂, —NH(C₁₋₅ alkyl), —N(C₁₋₅ alkyl)(C₁₋₅ alkyl), —NH—OH, —N(C₁₋₅ alkyl)-OH, —NH—O(C₁₋₅ alkyl), —N(C₁₋₅ alkyl)-O(C₁₋₅ alkyl), halogen, C₁₋₅ haloalkyl, —O(C₁₋₅ haloalkyl), —CN, —NO₂, —CHO, —CO(C₁₋₅ alkyl), —COOH, —COO(C₁₋₅ alkyl), —O—CO(C₁₋₅ alkyl), —CO—NH₂, —CO—NH(C₁₋₅ alkyl), —CO—N(C₁₋₅ alkyl)(C₁₋₅ alkyl), —NH—CO(C₁₋₅ alkyl), —N(C₁₋₅ alkyl)-CO(C₁₋₅ alkyl), —NH—COO(C₁₋₅ alkyl), —N(C₁₋₅ alkyl)-COO(C₁₋₅ alkyl), —O—CO—NH(C₁₋₅ alkyl), —O—CO—N(C₁₋₅ alkyl)(C₁₋₅ alkyl), —SO₂—NH₂, —SO₂—NH(C₁₋₅ alkyl), —SO₂—N(C₁₋₅ alkyl)(C₁₋₅ alkyl), —NH—SO₂—(C₁₋₅ alkyl), —N(C₁₋₅ alkyl)-SO₂—(C₁₋₅ alkyl), —SO₂—(C₁₋₅ alkyl), —SO—(C_{1- 5} alkyl), aryl, heteroaryl, cycloalkyl, and heterocycloalkyl, wherein said aryl, said heteroaryl, said cycloalkyl, and said heterocycloalkyl are each optionally substituted with one or more (e.g., one, two or three) groups independently selected from C₁₋₅ alkyl, C₂₋₅ alkenyl, C₂₋₅ alkynyl, halogen, C₁₋₅ haloalkyl, —O(C₁₋₅ haloalkyl), —CN, —OH, —O(C₁₋₅ alkyl), —SH, —S(C₁₋₅ alkyl), —NH₂, —NH(C₁₋₅ alkyl), and —N(C₁₋₅ alkyl)(C₁₋₅ alkyl).

Furthermore, in accordance with the present invention, the following compounds are excluded from formula (I):
1-(((5-phenyl-4,5-dihydro-1H-imidazol-2-yl)thio)methyl)pyrrolidine;
1-(2-((5-phenyl-4,5-dihydro-1H-imidazol-2-yl)thio)ethyl)pyrrolidine;
1-(3-((5-phenyl-4,5-dihydro-1H-imidazol-2-yl)thio)propyl)pyrrolidine;
1-(((5-phenyl-4,5-dihydro-1H-imidazol-2-yl)thio)methyl)piperidine;
1-(2-((5-phenyl-4,5-dihydro-1H-imidazol-2-yl)thio)ethyl)piperidine;
1-(3-((5-phenyl-4,5-dihydro-1H-imidazol-2-yl)thio)propyl)piperidine;
1-(2-((4,5-dihydro-1H-imidazol-2-yl)thio)ethyl)pyrrolidine;
1-(3-((4,5-dihydro-1H-imidazol-2-yl)thio)propyl)pyrrolidine;
1-(4-((4,5-dihydro-1H-imidazol-2-yl)thio)butyl)pyrrolidine;
1-(2-((4,5-dihydro-1H-imidazol-2-yl)thio)ethyl)piperidine;
1-(3-((4,5-dihydro-1H-imidazol-2-yl)thio)propyl)piperidine;
1-(4-((4,5-dihydro-1H-imidazol-2-yl)thio)butyl)piperidine;
1-(2-((4,5-dihydro-1H-imidazol-2-yl)thio)ethyl)azepane;
1-(3-((4,5-dihydro-1H-imidazol-2-yl)thio)propyl)azepane;
1-(4-((4,5-dihydro-1H-imidazol-2-yl)thio)butyl)azepane;
1-(2-((1,4,5,6-tetrahydropyrimidin-2-yl)thio)ethyl)pyrrolidine;
1-(3-((1,4,5,6-tetrahydropyrimidin-2-yl)thio)propyl)pyrrolidine;
1-(4-((1,4,5,6-tetrahydropyrimidin-2-yl)thio)butyl)pyrrolidine;
1-(2-((1,4,5,6-tetrahydropyrimidin-2-yl)thio)ethyl)piperidine;
1-(3-((1,4,5,6-tetrahydropyrimidin-2-yl)thio)propyl)piperidine;
1-(4-((1,4,5,6-tetrahydropyrimidin-2-yl)thio)butyl)piperidine;
1-(2-((1,4,5,6-tetrahydropyrimidin-2-yl)thio)ethyl)azepane;
1-(3-((1,4,5,6-tetrahydropyrimidin-2-yl)thio)propyl)azepane;
1-(4-((1,4,5,6-tetrahydropyrimidin-2-yl)thio)butyl)azepane;
1-(2-((4,5,6,7-tetrahydro-1H-1,3-diazepin-2-yl)thio)ethyl)pyrrolidine;
1-(3-((4,5,6,7-tetrahydro-1H-1,3-diazepin-2-yl)thio)propyl)pyrrolidine;
1-(4-((4,5,6,7-tetrahydro-1H-1,3-diazepin-2-yl)thio)butyl)pyrrolidine;
1-(2-((4,5,6,7-tetrahydro-1H-1,3-diazepin-2-yl)thio)ethyl)piperidine;
1-(3-((4,5,6,7-tetrahydro-1H-1,3-diazepin-2-yl)thio)propyl)piperidine;
1-(4-((4,5,6,7-tetrahydro-1H-1,3-diazepin-2-yl)thio)butyl)piperidine;
1-(2-((4,5,6,7-tetrahydro-1H-1,3-diazepin-2-yl)thio)ethyl)azepane;
1-(3-((4,5,6,7-tetrahydro-1H-1,3-diazepin-2-yl)thio)propyl)azepane;
1-(4-((4,5,6,7-tetrahydro-1H-1,3-diazepin-2-yl)thio)butyl)azepane;
2-((4,5-dihydro-1H-imidazol-2-yl)thio)-1-(pyridin-4-yl)ethan-1-one;
3-(1-(3-((4,5-dihydro-1H-imidazol-2-yl)thio)propyl)-1H-indol-3-yl)-4-(1-methyl-1H-indol-3-yl)-1H-pyrrole-2,5-dione;
2-((4,5-dihydro-1H-imidazol-2-yl)thio)-1-(pyridin-4-yl)ethanone;
3-((1,4,5,6-tetrahydropyrimidin-2-yl)thio)-1H-pyrrolo[2,3-b]pyridine; and
3-((3,4-dihydroquinazolin-2-yl)thio)-1H-indole-2-carboxylic acid.

Accordingly, the present invention does not relate to the compounds listed in the preceding paragraph or pharmaceutically acceptable salts or solvates thereof.

The following compounds are preferably also excluded from formula (I):
2-(cyclopentylthio)-4,5-dihydro-1H-imidazole;
N-(piperidinomethyl)-2-[(piperidinomethyl)thio]-2-imidazoline;
N-((2-methylpiperidino)methyl)-2-[((2-methylpiperidino)methyl)thio]-2-imidazoline;
N-((3-methylpiperidino)methyl)-2-[((3-methylpiperidino)methyl)thio]-2-imidazoline;
N-((4-methylpiperidino)methyl)-2-[((4-methylpiperidino)methyl)thio]-2-imidazoline; and
N-((2-methyl-5-ethylpiperidino)methyl)-2-[((2-methyl-5-ethylpiperidino)methyl)thio]-2-imidazoline.

Accordingly, it is preferred that the invention does not relate to the aforementioned compounds or pharmaceutically acceptable salts or solvates thereof.

It is furthermore preferred that if ring A is 2-imidazolin-2-yl (which is optionally substituted with one or more groups R^{A2}), if n is 0, if L is —CH₂—, and if ring B is piperidin-1-yl (which is optionally substituted with one or more groups R^{B1}), then R^{A2} is not piperidin-1-ylmethyl, wherein the piperidine group in said piperidin-1-ylmethyl is optionally substituted with one or more groups selected independently from methyl and ethyl.

Moreover, it is preferred that if ring A is a group A1 (which is optionally substituted with one or more groups R^{A2}), if n is 0, if L is —(CH₂)₂₋₄—, and if ring B is a group

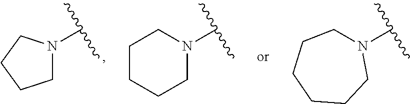 [SMILES: *N1CCCC1 ; *N1CCCCC1 ; *N1CCCCCC1]

(each of which is optionally substituted with one or more groups R^{B1}), then (i) the group R^{A1} is not hydrogen and/or (ii) the group A1 is substituted with at least one group (e.g., one, two or three groups) R^{A2} and/or (iii) ring B is substituted with at least one group (e.g., one, two or three groups) R^{B1}.

Accordingly, if ring A is a group A1 (which is optionally substituted with one or more R^{A2}), n is 0, L is —(CH₂)₂₋₄—, and ring B is a group

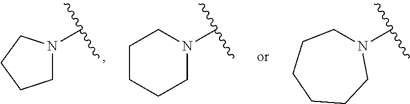 [SMILES: *N1CCCC1 ; *N1CCCCC1 ; *N1CCCCCC1]

(each of which is optionally substituted with one or more R^{B1}), then it is preferred that at least one of the following conditions applies: (i) the group R^{A1} is not hydrogen; (ii) the group A1 is substituted with one or more (e.g., one, two or three) groups R^{A2}; and/or (iii) ring B is substituted with one or more (e.g., one, two or three) groups R^{B1}.

Additionally or alternatively, it is preferred that if ring A is a group A1 which is substituted on a carbon ring atom with a phenyl group (wherein said group A1 is optionally further substituted with one or more groups R^{A2}), if n is 0, if L is —(CH₂)₁₋₃—, and if ring B is a group

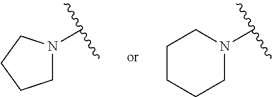 [SMILES: *N1CCCC1 ; *N1CCCCC1]

(each of which is optionally substituted with one or more groups R^{B1}), then (i) the group R^{A1} is not hydrogen and/or (ii) the group A1 is substituted with one or more further groups R^{A2} (besides the phenyl substituent) and/or (iii) ring B is substituted with one or more (e.g., one, two or three) groups R^{B1}.

More preferably, if ring A is a group A1 (which is optionally substituted with one or more groups R^{A2}) and ring B is a group

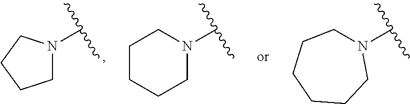 [SMILES: *N1CCCC1 ; *N1CCCCC1 ; *N1CCCCCC1]

(each of which is optionally substituted with one or more groups R^{B1}), then (i) the group R^{A1} is not hydrogen and/or (ii) the group A1 is substituted with at least one group R^{A2} which is different from phenyl (i.e., the group A1 is substituted with one group R^{A2} which is not phenyl, and is optionally further substituted with one or more additional groups R^{A2} which may also include phenyl) and/or (iii) ring B is substituted with one or more (e.g., one, two or three) groups R^{B1}.

It is particularly preferred that the compound of formula (I) is one of the specific compounds of formula (I) described in the examples section of this specification, including any one of Examples 1 to 200 described further below, either in non-salt form or as a pharmaceutically acceptable salt (e.g., a hydrochloride salt) or solvate of the respective compound.

Accordingly, it is particularly preferred that the compound of formula (I) is selected from:
3-(((6-chloro-1,4-dihydroquinazolin-2-yl)thio)methyl)-6,6-dimethyl-5,6-dihydroimidazo[2,1-b]thiazole;
7-chloro-3-(((5,5-dimethyl-4,5-dihydro-1H-imidazol-2-yl)thio)methyl)-5H-thiazolo[2,3-b]quinazoline;
3-(((3,4-dihydroquinazolin-2-yl)thio)methyl)-6,6-dimethyl-5,6-dihydroimidazo[2,1-b]thiazole;
7-chloro-3-(((4,5-dihydro-1H-imidazol-2-yl)thio)methyl)-5H-thiazolo[2,3-b]quinazoline;
3-(((5,5-dimethyl-4,5-dihydro-1H-imidazol-2-yl)thio)methyl)-5H-thiazolo[2,3-b]quinazoline;
3-(((7-chloro-1,4-dihydroquinazolin-2-yl)thio)methyl)-6,6-dimethyl-5,6-dihydroimidazo[2,1-b]thiazole;
3-(((2,5-dihydro-1H-benzo[e][1,3]diazepin-3-yl)thio)methyl)-6,6-dimethyl-5,6-dihydroimidazo[2,1-b]thiazole;
3-(((5,5-dimethyl-4,5-dihydro-1H-imidazol-2-yl)thio)methyl)-5,10-dihydrobenzo[e]thiazolo[3,2-a][1,3]diazepine;
8-chloro-3-(((5,5-dimethyl-4,5-dihydro-1H-imidazol-2-yl)thio)methyl)-5H-thiazolo[2,3-b]quinazoline;
3-(((6-chloro-1,4-dihydroquinazolin-2-yl)thio)methyl)-5,6-dihydroimidazo[2,1-b]thiazole;
3-(((3,4-dihydroquinazolin-2-yl)thio)methyl)-5,10-dihydrobenzo[e]thiazolo[3,2-a][1,3]diazepine;
3-(((1,4-dihydroquinazolin-2-yl)thio)methyl)-5H-thiazolo[2,3-b]quinazoline;
trans-3-(((3,4-dihydroquinazolin-2-yl)thio)methyl)-4a,5,6,7,8,8a-hexahydrobenzo[4,5]imidazo[2,1-b]thiazole;
6-(4-chlorophenyl)-3-(((3,4-dihydroquinazolin-2-yl)thio)methyl)-5,6-dihydroimidazo[2,1-b]thiazole;
6-cyclohexyl-3-(((3,4-dihydroquinazolin-2-yl)thio)methyl)-5,6-dihydroimidazo[2,1-b]thiazole;
trans-3-(((3,4-dihydroquinazolin-2-yl)thio)methyl)-5,6-diphenyl-5,6-dihydroimidazo[2,1-b]thiazole;
trans-3-(((3,4-dihydroquinazolin-2-yl)thio)methyl)-5,6-diphenyl-2,3,5,6-tetrahydroimidazo[2,1-b]thiazol-3-ol;
3-(((3,4-dihydroquinazolin-2-yl)thio)methyl)-6-fluoro-5H-thiazolo[2,3-b]quinazoline;
7-chloro-3-(((1,4-dihydroquinazolin-2-yl)thio)methyl)-5H-thiazolo[2,3-b]quinazoline;
3-(((5-benzyl-4,5-dihydro-1H-imidazol-2-yl)thio)methyl)-7-chloro-5H-thiazolo[2,3-b]quinazoline;
3-(((4,4-dimethyl-1,4-dihydroquinazolin-2-yl)thio)methyl)-5,10-dihydrobenzo[e]thiazolo[3,2-a][1,3]diazepine;
3-(((4-(4-chlorophenyl)-4,5-dihydro-1H-imidazol-2-yl)thio)methyl)-5,10-dihydrobenzo[e]thiazolo[3,2-a][1,3]diazepine;
3-(((5-fluoro-1,4-dihydroquinazolin-2-yl)thio)methyl)-5,10-dihydrobenzo[e]thiazolo[3,2-a][1,3]diazepine;
3-((((4S,5S)-4,5-diphenyl-4,5-dihydro-1H-imidazol-2-yl)thio)methyl)-5,10-dihydrobenzo[e]thiazolo[3,2-a][1,3]diazepine;
3-(((4,5-dihydro-1H-benzo[d][1,3]diazepin-2-yl)thio)methyl)-5,10-dihydrobenzo[e]thiazolo[3,2-a][1,3]diazepine;
3-(((4-cyclohexyl-4,5-dihydro-1H-imidazol-2-yl)thio)methyl)-5,10-dihydrobenzo[e]thiazolo[3,2-a][1,3]diazepine;
3-(((4-phenyl-3,4-dihydroquinazolin-2-yl)thio)methyl)-5,10-dihydrobenzo[e]thiazolo[3,2-a][1,3]diazepine;
3-(((3,4-dihydroquinazolin-2-yl)thio)methyl)-5H-pyrido[2,3-d]thiazolo[3,2-a]pyrimidine;
3-(((5-butyl-4,5-dihydro-1H-imidazol-2-yl)thio)methyl)-5H-thiazolo[2,3-b]quinazoline;
3-(((5-methyl-5-phenyl-4,5-dihydro-1H-imidazol-2-yl)thio)methyl)-5,10-dihydrobenzo[e]thiazolo[3,2-a][1,3]diazepine;
3-(((1,4-dihydropyrido[2,3-d]pyrimidin-2-yl)thio)methyl)-5,10-dihydrobenzo[e]thiazolo[3,2-a][1,3]diazepine;
3-((((3aR,7aR)-3a,4,5,6,7,7a-hexahydro-1H-benzo[d]imidazol-2-yl)thio)methyl)-5H-thiazolo[2,3-b]quinazoline;
3-(((5-benzyl-4,5-dihydro-1H-imidazol-2-yl)thio)methyl)-5H-thiazolo[2,3-b]quinazoline;
3-(((5-(4-methoxybenzyl)-5-methyl-4,5-dihydro-1H-imidazol-2-yl)thio)methyl)-5,10-dihydrobenzo[e]thiazolo[3,2-a][1,3]diazepine;
3-(((3,4-dihydroquinazolin-2-yl)thio)methyl)-5,6-dihydrobenzo[d]thiazolo[3,2-a][1,3]diazepine;
3-(((1-methyl-4,5-dihydro-1H-imidazol-2-yl)thio)methyl)-5H-thiazolo[2,3-b]quinazoline;
3-(((1-butyl-4,5-dihydro-1H-imidazol-2-yl)thio)methyl)-5,10-dihydrobenzo[e]thiazolo[3,2-a][1,3]diazepine;
3-(((3,4-dihydroquinazolin-2-yl)thio)methyl)-6-methyl-6-phenyl-5,6-dihydroimidazo[2,1-b]thiazole;
3-(((6-chloro-1,4-dihydroquinazolin-2-yl)thio)methyl)-5H-thiazolo[2,3-b]quinazoline;
7-chloro-3-((((3aR,7aR)-3a,4,5,6,7,7a-hexahydro-1H-benzo[d]imidazol-2-yl)thio)methyl)-5H-thiazolo[2,3-b]quinazoline;
3-(((5-butyl-4,5-dihydro-1H-imidazol-2-yl)thio)methyl)-7-chloro-5H-thiazolo[2,3-b]quinazoline;
8-chloro-3-(((1,4-dihydroquinazolin-2-yl)thio)methyl)-5H-thiazolo[2,3-b]quinazoline;
3-(((3,4-dihydroquinazolin-2-yl)thio)methyl)-5-phenyl-5H-thiazolo[2,3-b]quinazoline;
7-chloro-3-(((6-chloro-1,4-dihydroquinazolin-2-yl)thio)methyl)-5H-thiazolo[2,3-b]quinazoline;
7-chloro-3-(((1-methyl-4,5-dihydro-1H-imidazol-2-yl)thio)methyl)-5H-thiazolo[2,3-b]quinazoline;
3-(((3,4-dihydroquinazolin-2-yl)thio)methyl)-6-(4-methoxybenzyl)-6-methyl-5,6-dihydroimidazo[2,1-b]thiazole;
3-(((1-benzyl-4,5-dihydro-1H-imidazol-2-yl)thio)methyl)-7-chloro-5H-thiazolo[2,3-b]quinazoline;
7-chloro-3-(((1-isopropyl-4,5-dihydro-1H-imidazol-2-yl)thio)methyl)-5H-thiazolo[2,3-b]quinazoline;
7-chloro-3-(((1,5,6,7,8,8a-hexahydroimidazo[1,5-a]pyridin-3-yl)thio)methyl)-5H-thiazolo[2,3-b]quinazoline;
1-(2-((5-benzyl-4,5-dihydro-1H-imidazol-2-yl)thio)ethyl)piperidine;
2-(((5-benzyl-4,5-dihydro-1H-imidazol-2-yl)thio)methyl)imidazo[1,2-a]pyrimidine;
5-benzyl-2-((3-(pyrrolidin-1-yl)propyl)thio)-4,5-dihydro-1H-imidazole;
5-benzyl-2-(((1-methylpyrrolidin-2-yl)methyl)thio)-4,5-dihydro-1H-imidazole;
5-benzyl-2-((2-(pyrrolidin-1-yl)ethyl)thio)-4,5-dihydro-1H-imidazole;
4-(3-((5-benzyl-4,5-dihydro-1H-imidazol-2-yl)thio)propyl)pyridine;
4-(((5-benzyl-4,5-dihydro-1H-imidazol-2-yl)thio)methyl)pyridine;
5-benzyl-2-((2-(1-methylpyrrolidin-2-yl)ethyl)thio)-4,5-dihydro-1H-imidazole;
1-(2-((5-benzyl-4,5-dihydro-1H-imidazol-2-yl)thio)ethyl)azepane;
6-chloro-2-((2-(pyrrolidin-1-yl)ethyl)thio)-1,4-dihydroquinazoline;
6-chloro-2-((4-(pyrrolidin-1-yl)butyl)thio)-1,4-dihydroquinazoline;
2-(((5-benzyl-4,5-dihydro-1H-imidazol-2-yl)thio)methyl)-4-chlorothieno[3,2-c]pyridine;
3-(((3,4-dihydroquinazolin-2-yl)thio)methyl)-6,7-dimethoxy-2,3-dihydrobenzo[4,5]imidazo[2,1-b]thiazol-3-ol;
3-(((3,4-dihydroquinazolin-2-yl)thio)methyl)-6-(thiophen-2-ylmethyl)-5,6-dihydroimidazo[2,1-b]thiazole;
7-chloro-3-(((5-(thiophen-2-ylmethyl)-4,5-dihydro-1H-imidazol-2-yl)thio)methyl)-5H-thiazolo[2,3-b]quinazoline;
6-benzyl-3-(((3,4-dihydroquinazolin-2-yl)thio)methyl)-5,6-dihydroimidazo[2,1-b]thiazole;
3-(((7-bromo-1,4-dihydroquinazolin-2-yl)thio)methyl)-7-chloro-5H-thiazolo[2,3-b]quinazoline;
3-(((6-bromo-1,4-dihydroquinazolin-2-yl)thio)methyl)-7-chloro-5H-thiazolo[2,3-b]quinazoline;
3-(((4,6-diazaspiro[2.4]hept-5-en-5-yl)thio)methyl)-7-chloro-5H-thiazolo[2,3-b]quinazoline;
7-bromo-3-(((1,4-dihydroquinazolin-2-yl)thio)methyl)-5H-thiazolo[2,3-b]quinazoline;
8-bromo-3-(((1,4-dihydroquinazolin-2-yl)thio)methyl)-5H-thiazolo[2,3-b]quinazoline;
2-((2-(isoindolin-2-yl)ethyl)thio)-3,4-dihydroquinazoline;
7-chloro-3-(((5-methyl-5-phenyl-4,5-dihydro-1H-imidazol-2-yl)thio)methyl)-5H-thiazolo[2,3-b]quinazoline;
3-(((4,4-dimethyl-1,4-dihydroquinazolin-2-yl)thio)methyl)-6-fluoro-5H-thiazolo[2,3-b]quinazoline;
2-((2-(5-chloro-1H-indol-1-yl)ethyl)thio)-3,4-dihydroquinazoline;
7-chloro-3-(((4,4-dimethyl-1,4-dihydroquinazolin-2-yl)thio)methyl)-5H-thiazolo[2,3-b]quinazoline;
7-chloro-3-(((4,5-dihydro-1H-benzo[d][1,3]diazepin-2-yl)thio)methyl)-5H-thiazolo[2,3-b]quinazoline;
2-((2-(pyrrolidin-1-yl)ethyl)thio)-1,4-dihydroquinazoline;
4,4-dimethyl-2-((2-(pyrrolidin-1-yl)ethyl)thio)-1,4-dihydroquinazoline;
2-bromo-7-chloro-3-(((1,4-dihydroquinazolin-2-yl)thio)methyl)-5H-thiazolo[2,3-b]quinazoline;
7-chloro-3-(((5-fluoro-1,4-dihydroquinazolin-2-yl)thio)methyl)-5H-thiazolo[2,3-b]quinazoline;
6-chloro-3-(((1,4-dihydroquinazolin-2-yl)thio)methyl)-5H-thiazolo[2,3-b]quinazoline;
3-(((1,4-dihydroquinazolin-2-yl)thio)methyl)-8-fluoro-5H-thiazolo[2,3-b]quinazoline;
7-chloro-3-(((6-fluoro-1,4-dihydroquinazolin-2-yl)thio)methyl)-5H-thiazolo[2,3-b]quinazoline;
3-(((1,4-dihydroquinazolin-2-yl)thio)methyl)-7-fluoro-5H-thiazolo[2,3-b]quinazoline;
9-bromo-3-(((1,4-dihydroquinazolin-2-yl)thio)methyl)-5H-thiazolo[2,3-b]quinazoline;
7-chloro-3-(((1,4-dihydroquinazolin-2-yl)thio)methyl)-9-fluoro-5H-thiazolo[2,3-b]quinazoline;
6-benzyl-3-(((4,4-dimethyl-1,4-dihydroquinazolin-2-yl)thio)methyl)-5,6-dihydroimidazo[2,1-b]thiazole;
6-benzyl-3-(((4,5-dihydro-1H-benzo[d][1,3]diazepin-2-yl)thio)methyl)-5,6-dihydroimidazo[2,1-b]thiazole;
6-benzyl-3-(((7-fluoro-1,4-dihydroquinazolin-2-yl)thio)methyl)-5,6-dihydroimidazo[2,1-b]thiazole;
2-((2-(azepan-1-yl)ethyl)thio)-1,4-dihydroquinazoline;
2-((2-(piperidin-1-yl)ethyl)thio)-1,4-dihydroquinazoline;
3-(((8-bromo-1,4-dihydroquinazolin-2-yl)thio)methyl)-7-chloro-5H-thiazolo[2,3-b]quinazoline;
6-benzyl-3-(((3-butyl-3,4-dihydroquinazolin-2-yl)thio)methyl)-5,6-dihydroimidazo[2,1-b]thiazole;
6-(4-chlorobenzyl)-3-(((1,4-dihydroquinazolin-2-yl)thio)methyl)-5,6-dihydroimidazo[2,1-b]thiazole;
3-(((1,4-dihydroquinazolin-2-yl)thio)methyl)-5,5-dimethyl-5H-thiazolo[2,3-b]quinazoline;
3-(((3,4-dihydroquinazolin-2-yl)thio)methyl)benzo[4,5]imidazo[2,1-b]thiazole;
3-(((3,4-dihydroquinazolin-2-yl)thio)methyl)-6,7-dimethoxybenzo[4,5]imidazo[2,1-b]thiazole;
4,4-dimethyl-2-((1-methylpyrrolidin-3-yl)thio)-1,4-dihydroquinazoline;
6-benzyl-3-(((1-butyl-1,4-dihydroquinazolin-2-yl)thio)methyl)-5,6-dihydroimidazo[2,1-b]thiazole;
2-((1-methylpyrrolidin-3-yl)thio)-1,4-dihydroquinazoline;
2-((1-phenylpyrrolidin-3-yl)thio)-1,4-dihydroquinazoline;
2-((1-(2,2-difluoroethyl)pyrrolidin-3-yl)thio)-1,4-dihydroquinazoline;
6-chloro-2-((1-methylpyrrolidin-3-yl)thio)-1,4-dihydroquinazoline;
2-((1-ethylpyrrolidin-3-yl)thio)-1,4-dihydroquinazoline;
2-((1-methylpyrrolidin-3-yl)thio)-4,5-dihydro-1H-benzo[d][1,3]diazepine;
3-((1-phenylpyrrolidin-3-yl)thio)-2,5-dihydro-1H-benzo[e][1,3]diazepine;
2-((1-phenylpyrrolidin-3-yl)thio)-4,5-dihydro-1H-benzo[d][1,3]diazepine;
2-(((1-methylpyrrolidin-2-yl)methyl)thio)-1,4-dihydroquinazoline;
(S)-6-((1H-indol-3-yl)methyl)-3-(((1,4-dihydroquinazolin-2-yl)thio)methyl)-5,6-dihydroimidazo[2,1-b]thiazole;
6-benzyl-3-(((1,4-dihydroquinazolin-2-yl)thio)methyl)-2-iodo-5,6-dihydroimidazo[2,1-b]thiazole;
(S)-6-(3-chlorobenzyl)-3-(((1,4-dihydroquinazolin-2-yl)thio)methyl)-5,6-dihydroimidazo[2,1-b]thiazole;
3-(((1,4-dihydroquinazolin-2-yl)thio)methyl)-6-(3-methylbenzyl)-5,6-dihydroimidazo[2,1-b]thiazole;
6-benzyl-3-(((4-methyl-1,4-dihydroquinazolin-2-yl)thio)methyl)-5,6-dihydroimidazo[2,1-b]thiazole;
6-benzyl-3-(((6-chloro-1,4-dihydroquinazolin-2-yl)thio)methyl)-5,6-dihydroimidazo[2,1-b]thiazole;
2-((2-(indolin-1-yl)ethyl)thio)-1,4-dihydroquinazoline;
4-chloro-2-(((1,4-dihydroquinazolin-2-yl)thio)methyl)thieno[3,2-c]pyridine;
6-benzyl-3-(((5-fluoro-1,4-dihydroquinazolin-2-yl)thio)methyl)-5,6-dihydroimidazo[2,1-b]thiazole;
6-benzyl-3-(((5-chloro-1,4-dihydroquinazolin-2-yl)thio)methyl)-5,6-dihydroimidazo[2,1-b]thiazole;
6-benzyl-3-(((7-bromo-1,4-dihydroquinazolin-2-yl)thio)methyl)-5,6-dihydroimidazo[2,1-b]thiazole;
3-(((1,4-dihydroquinazolin-2-yl)thio)methyl)-6-phenyl-5,6-dihydroimidazo[2,1-b]thiazole;
3-(((1,4-dihydroquinazolin-2-yl)thio)methyl)-6-(3-fluorobenzyl)-5,6-dihydroimidazo[2,1-b]thiazole;
3-(((1,4-dihydroquinazolin-2-yl)thio)methyl)-6-(4-methylbenzyl)-5,6-dihydroimidazo[2,1-b]thiazole;
6-(2-chlorobenzyl)-3-(((1,4-dihydroquinazolin-2-yl)thio)methyl)-5,6-dihydroimidazo[2,1-b]thiazole;
(R)-3-(((1,4-dihydroquinazolin-2-yl)thio)methyl)-6-(4-methoxybenzyl)-5,6-dihydroimidazo[2,1-b]thiazole;
2-((2-(3,3-difluoropyrrolidin-1-yl)ethyl)thio)-1,4-dihydroquinazoline;
3-(((1,4-dihydroquinazolin-2-yl)thio)methyl)-6-phenethyl-5,6-dihydroimidazo[2,1-b]thiazole;
2-((2-(3-methoxypyrrolidin-1-yl)ethyl)thio)-1,4-dihydroquinazoline;
2-((2-(2-phenylpyrrolidin-1-yl)ethyl)thio)-1,4-dihydroquinazoline;
2-((2-(pyrrolidin-1-yl)propyl)thio)-1,4-dihydroquinazoline;
2-((2-(2-methylpyrrolidin-1-yl)ethyl)thio)-1,4-dihydroquinazoline;
5-methyl-5-phenyl-2-((2-(pyrrolidin-1-yl)ethyl)thio)-4,5-dihydro-1H-imidazole;
2-((2-(1,1-difluoro-5-azaspiro[2.4]heptan-5-yl)ethyl)thio)-3,4-dihydroquinazoline;
2-((2-((1R,5S)-8-azabicyclo[3.2.1]octan-8-yl)ethyl)thio)-3,4-dihydroquinazoline;
6,7,8-triiodo-2-((2-(pyrrolidin-1-yl)ethyl)thio)-1,4-dihydroquinazoline;
1-(2-((1,4-dihydroquinazolin-2-yl)thio)ethyl)pyrrolidin-2-one;
2-((3-(pyrrolidin-1-yl)propyl)thio)-1,4-dihydroquinazoline;
2-((4-(pyrrolidin-1-yl)butyl)thio)-1,4-dihydroquinazoline;
2-((2-(3-methylpyrrolidin-1-yl)ethyl)thio)-1,4-dihydroquinazoline;
(1S,4S)-5-(2-((1,4-dihydroquinazolin-2-yl)thio)ethyl)-2-oxa-5-azabicyclo[2.2.1]heptane;
2-((2-(3-phenylpyrrolidin-1-yl)ethyl)thio)-1,4-dihydroquinazoline;
2-(((2R)-2-(pyrrolidin-1-yl)cyclopentyl)thio)-1,4-dihydroquinazoline;
2-((2-(2-azaspiro[4.4]nonan-2-yl)ethyl)thio)-1,4-dihydroquinazoline;
2-((2-(3-(benzyloxy)pyrrolidin-1-yl)ethyl)thio)-1,4-dihydroquinazoline;
1-(2-((1,4-dihydroquinazolin-2-yl)thio)ethyl)pyrrolidine-3-carboxylic acid;
2-((2-(1-methylpyrrolidin-3-yl)ethyl)thio)-1,4-dihydroquinazoline;
(1R,4R)-5-(2-((1,4-dihydroquinazolin-2-yl)thio)ethyl)-2-oxa-5-azabicyclo[2.2.1]heptane;
4-((1,4-dihydroquinazolin-2-yl)thio)-1-(pyrrolidin-1-yl)butan-1-one;
2-(((2R)-2-(pyrrolidin-1-yl)cyclohexyl)thio)-1,4-dihydroquinazoline;
5-fluoro-2-((4-(pyrrolidin-1-yl)butyl)thio)-1,4-dihydroquinazoline;
7-chloro-2-((4-(pyrrolidin-1-yl)butyl)thio)-1,4-dihydroquinazoline;
7-fluoro-2-((4-(pyrrolidin-1-yl)butyl)thio)-1,4-dihydroquinazoline;
6-fluoro-2-((4-(pyrrolidin-1-yl)butyl)thio)-1,4-dihydroquinazoline;
8-chloro-2-((4-(pyrrolidin-1-yl)butyl)thio)-1,4-dihydroquinazoline;
2-((2-(3-benzylpyrrolidin-1-yl)ethyl)thio)-1,4-dihydroquinazoline;
4-(2-((1,4-dihydroquinazolin-2-yl)thio)ethyl)morpholine;
(S)-2-((2-(3-fluoropyrrolidin-1-yl)ethyl)thio)-1,4-dihydroquinazoline;
(R)-2-((2-(3-fluoropyrrolidin-1-yl)ethyl)thio)-1,4-dihydroquinazoline;
6-chloro-2-((2-(1-methylpyrrolidin-2-yl)ethyl)thio)-1,4-dihydroquinazoline;
2-((4-(pyrrolidin-1-yl)butyl)thio)-4,5-dihydro-1H-benzo[d][1,3]diazepine;
4,4-dimethyl-2-((4-(pyrrolidin-1-yl)butyl)thio)-1,4-dihydroquinazoline;
6-chloro-2-((3-(pyrrolidin-1-yl)propyl)thio)-1,4-dihydroquinazoline;
6-chloro-2-((4-(pyrrolidin-1-yl)pentyl)thio)-1,4-dihydroquinazoline;
6-bromo-2-((4-(pyrrolidin-1-yl)butyl)thio)-1,4-dihydroquinazoline;
6-chloro-2-((4-(piperidin-1-yl)butyl)thio)-1,4-dihydroquinazoline;
2-((4-(pyrrolidin-1-yl)pentyl)thio)-1,4-dihydroquinazoline;
(S)-6-chloro-2-((2-(pyrrolidin-1-yl)propyl)thio)-1,4-dihydroquinazoline;
(R)-6-chloro-2-((2-(pyrrolidin-1-yl)propyl)thio)-1,4-dihydroquinazoline;
(S)-6-chloro-2-((1-(pyrrolidin-1-yl)propan-2-yl)thio)-1,4-dihydroquinazoline;
5-(4-methoxybenzyl)-5-methyl-2-((4-(pyrrolidin-1-yl)butyl)thio)-4,5-dihydro-1H-imidazole;
5-methyl-5-phenyl-2-((4-(pyrrolidin-1-yl)butyl)thio)-4,5-dihydro-1H-imidazole;
3-((4-(pyrrolidin-1-yl)butyl)thio)-2,5-dihydro-1H-benzo[e][1,3]diazepine;
4,4-dimethyl-2-((4-(pyrrolidin-1-yl)butyl)thio)-4,5-dihydro-1H-imidazole;
2-((4-(pyrrolidin-1-yl)butyl)thio)-1,4,5,6-tetrahydropyrimidine;
6-chloro-2-((3-(1-methylpyrrolidin-2-yl)propyl)thio)-1,4-dihydroquinazoline;
2-((4-(pyrrolidin-1-yl)butyl)thio)-4,5-dihydro-1H-imidazole;
2-((4-(1H-imidazol-1-yl)butyl)thio)-6-chloro-1,4-dihydroquinazoline;
6-chloro-2-((2-(1-methylpyrrolidin-3-yl)ethyl)thio)-1,4-dihydroquinazoline;
2-((4-(pyrrolidin-1-yl)butyl)thio)-4,5,6,7-tetrahydro-1H-1,3-diazepine;
5,5-dimethyl-2-((4-(pyrrolidin-1-yl)butyl)thio)-1,4,5,6-tetrahydropyrimidine;
2′-((4-(pyrrolidin-1-yl)butyl)thio)-1′H-spiro[cyclopropane-1,4′-quinazoline];
5-benzyl-2-((4-(pyrrolidin-1-yl)butyl)thio)-4,5-dihydro-1H-imidazole;
2-((2-(pyrrolidin-1-yl)ethyl)thio)-4,5-dihydro-1H-benzo[d][1,3]diazepine;
5-(4-methoxybenzyl)-5-methyl-2-((2-(pyrrolidin-1-yl)ethyl)thio)-4,5-dihydro-1H-imidazole;
2-((2-(pyrrolidin-1-yl)ethyl)thio)-1,4,4a,5,6,7,8,8a-octahydroquinazoline;
5-((4-(pyrrolidin-1-yl)butyl)thio)-4,6-diazaspiro[2.4]hept-5-ene;
3-((2-(pyrrolidin-1-yl)ethyl)thio)-2,5-dihydro-1H-benzo[e][1,3]diazepine;
5-((2-(pyrrolidin-1-yl)ethyl)thio)-4,6-diazaspiro[2.4]hept-5-ene;
2-((pyridin-4-ylmethyl)thio)-4,5-dihydro-1H-benzo[d][1,3]diazepine;
3-((pyridin-4-ylmethyl)thio)-2,5-dihydro-1H-benzo[e][1,3]diazepine;
2-((3-(pyrrolidin-1-yl)propyl)thio)-4,5-dihydro-3H-benzo[d][1,3]diazepine;
2-((2-(3,4-dihydroquinolin-1(2H)-yl)ethyl)thio)-4,5-dihydro-1H-benzo[d][1,3]diazepine;
2-((2-(indolin-1-yl)ethyl)thio)-4,5-dihydro-1H-benzo[d][1,3]diazepine;
3-((pyridin-3-ylmethyl)thio)-2,5-dihydro-1H-benzo[e][1,3]diazepine;
3-((3-(pyrrolidin-1-yl)propyl)thio)-2,5-dihydro-1H-benzo[e][1,3]diazepine;
3-((2-(indolin-1-yl)ethyl)thio)-2,5-dihydro-1H-benzo[e][1,3]diazepine;
3-(((1,4-dihydroquinazolin-2-yl)thio)methyl)-6-(4-fluorobenzyl)-5,6-dihydroimidazo[2,1-b]thiazole;
2-((2-cyclopentylethyl)thio)-1,4-dihydroquinazoline;
tert-butyl (S)-3-((4,5-dihydro-1H-benzo[d][1,3]diazepin-2-yl)thio)pyrrolidine-1-carboxylate;
(S)-2-(pyrrolidin-3-ylthio)-4,5-dihydro-3H-benzo[d][1,3]diazepine;
(S)-2-((1-methylpyrrolidin-3-yl)thio)-4,5-dihydro-3H-benzo[d][1,3]diazepine;
   and pharmaceutically acceptable salts and solvates of any one of the aforementioned compounds.

The present invention also relates to each of the intermediates described further below in the examples section of this specification, including any one of these intermediates in non-salt form or in the form of a salt (e.g., a pharmaceutically acceptable salt) of the respective compound. Such intermediates can be used, in particular, in the synthesis of the compounds of formula (I).

For a person skilled in the field of synthetic chemistry, various ways for the preparation of the compounds of formula (I) will be readily apparent. For example, the compounds of formula (I) can be prepared in accordance with or in analogy to the synthetic routes described in the examples section.

The following definitions apply throughout the present specification and the claims, unless specifically indicated otherwise.

The term “hydrocarbon group” refers to a group consisting of carbon atoms and hydrogen atoms.

The term “alicyclic” is used in connection with cyclic groups and denotes that the corresponding cyclic group is non-aromatic.

As used herein, the term “alkyl” refers to a monovalent saturated acyclic (i.e., non-cyclic) hydrocarbon group which may be linear or branched. Accordingly, an “alkyl” group does not comprise any carbon-to-carbon double bond or any carbon-to-carbon triple bond. A “C₁₋₅ alkyl” denotes an alkyl group having 1 to 5 carbon atoms. Preferred exemplary alkyl groups are methyl, ethyl, propyl (e.g., n-propyl or isopropyl), or butyl (e.g., n-butyl, isobutyl, sec-butyl, or tert-butyl). Unless defined otherwise, the term “alkyl” preferably refers to C₁₋₄ alkyl, more preferably to methyl or ethyl, and even more preferably to methyl.

As used herein, the term “alkenyl” refers to a monovalent unsaturated acyclic hydrocarbon group which may be linear or branched and comprises one or more (e.g., one or two) carbon-to-carbon double bonds while it does not comprise any carbon-to-carbon triple bond. The term “C₂₋₅ alkenyl” denotes an alkenyl group having 2 to 5 carbon atoms.

Preferred exemplary alkenyl groups are ethenyl, propenyl (e.g., prop-1-en-1-yl, prop-1-en-2-yl, or prop-2-en-1-yl), butenyl, butadienyl (e.g., buta-1,3-dien-1-yl or buta-1,3-dien-2-yl), pentenyl, or pentadienyl (e.g., isoprenyl). Unless defined otherwise, the term “alkenyl” preferably refers to C₂₋₄ alkenyl.

As used herein, the term “alkynyl” refers to a monovalent unsaturated acyclic hydrocarbon group which may be linear or branched and comprises one or more (e.g., one or two) carbon-to-carbon triple bonds and optionally one or more (e.g., one or two) carbon-to-carbon double bonds. The term “C₂₋₅ alkynyl” denotes an alkynyl group having 2 to 5 carbon atoms. Preferred exemplary alkynyl groups are ethynyl, propynyl (e.g., propargyl), or butynyl. Unless defined otherwise, the term “alkynyl” preferably refers to C₂₋₄ alkynyl.

As used herein, the term “alkylene” refers to an alkanediyl group, i.e. a divalent saturated acyclic hydrocarbon group which may be linear or branched. A “C₁₋₅ alkylene” denotes an alkylene group having 1 to 5 carbon atoms; the term “C₀₋₅ alkylene” indicates that a covalent bond (corresponding to the option “C₀ alkylene”) or a C₁₋₅ alkylene is present. Preferred exemplary alkylene groups are methylene (—CH₂—), ethylene (e.g., —CH₂—CH₂— or —CH(—CH₃)—), propylene (e.g., —CH₂—CH₂—CH₂—, —CH(—CH₂—CH₃)—, —CH₂—CH(—CH₃)—, or —CH(—CH₃)—CH₂—), or butylene (e.g., —CH₂—CH₂—CH₂—CH₂—). Unless defined otherwise, the term “alkylene” preferably refers to C₁₋₄ alkylene (including, in particular, linear C₁₋₄ alkylene), more preferably to methylene or ethylene, and even more preferably to methylene.

As used herein, the term “alkenylene” refers to an alkenediyl group, i.e. a divalent unsaturated acyclic hydrocarbon group which may be linear or branched and comprises one or more (e.g., one or two) carbon-to-carbon double bonds while it does not comprise any carbon-to-carbon triple bond. A “C₂₋₅ alkenylene” denotes an alkenylene group having 2 to 5 carbon atoms. Unless defined otherwise, the term “alkenylene” preferably refers to C₂₋₄ alkenylene (including, in particular, linear C₂₋₄ alkenylene).

As used herein, the term “alkynylene” refers to an alkynediyl group, i.e. a divalent unsaturated acyclic hydrocarbon group which may be linear or branched and comprises one or more (e.g., one or two) carbon-to-carbon triple bonds and optionally one or more (e.g., one or two) carbon-to-carbon double bonds. A “C₂₋₅ alkynylene” denotes an alkynylene group having 2 to 5 carbon atoms. Unless defined otherwise, the term “alkynylene” preferably refers to C₂₋₄ alkynylene (including, in particular, linear C₂₋₄ alkynylene).

As used herein, the term “carbocyclyl” refers to a hydrocarbon ring group, including monocyclic rings as well as bridged ring, spiro ring and/or fused ring systems (which may be composed, e.g., of two or three rings), wherein said ring group may be saturated, partially unsaturated (i.e., unsaturated but not aromatic) or aromatic. Unless defined otherwise, “carbocyclyl” preferably refers to aryl, cycloalkyl or cycloalkenyl.

As used herein, the term “carbocyclylene” refers to a carbocyclyl group, as defined herein above, but having two points of attachment, i.e. a divalent hydrocarbon ring group, including monocyclic rings as well as bridged ring, spiro ring and/or fused ring systems (which may be composed, e.g., of two or three rings), wherein said ring group may be saturated, partially unsaturated (i.e., unsaturated but not aromatic) or aromatic. Unless defined otherwise, “carbocyclylene” preferably refers to arylene, cycloalkylene or cycloalkenylene.

As used herein, the term “heterocyclyl” refers to a ring group, including monocyclic rings as well as bridged ring, spiro ring and/or fused ring systems (which may be composed, e.g., of two or three rings), wherein said ring group comprises one or more (such as, e.g., one, two, three, or four) ring heteroatoms independently selected from O, S and N, and the remaining ring atoms are carbon atoms, wherein one or more S ring atoms (if present) and/or one or more N ring atoms (if present) may optionally be oxidized, wherein one or more carbon ring atoms may optionally be oxidized (i.e., to form an oxo group), and further wherein said ring group may be saturated, partially unsaturated (i.e., unsaturated but not aromatic) or aromatic. For example, each heteroatom-containing ring comprised in said ring group may contain one or two O atoms and/or one or two S atoms (which may optionally be oxidized) and/or one, two, three or four N atoms (which may optionally be oxidized), provided that the total number of heteroatoms in the corresponding heteroatom-containing ring is 1 to 4 and that there is at least one carbon ring atom (which may optionally be oxidized) in the corresponding heteroatom-containing ring. Unless defined otherwise, “heterocyclyl” preferably refers to heteroaryl, heterocycloalkyl or heterocycloalkenyl.

As used herein, the term “heterocyclylene” refers to a heterocyclyl group, as defined herein above, but having two points of attachment, i.e. a divalent ring group, including monocyclic rings as well as bridged ring, spiro ring and/or fused ring systems (which may be composed, e.g., of two or three rings), wherein said ring group comprises one or more (such as, e.g., one, two, three, or four) ring heteroatoms independently selected from O, S and N, and the remaining ring atoms are carbon atoms, wherein one or more S ring atoms (if present) and/or one or more N ring atoms (if present) may optionally be oxidized, wherein one or more carbon ring atoms may optionally be oxidized (i.e., to form an oxo group), and further wherein said ring group may be saturated, partially unsaturated (i.e., unsaturated but not aromatic) or aromatic. For example, each heteroatom-containing ring comprised in said ring group may contain one or two O atoms and/or one or two S atoms (which may optionally be oxidized) and/or one, two, three or four N atoms (which may optionally be oxidized), provided that the total number of heteroatoms in the corresponding heteroatom-containing ring is 1 to 4 and that there is at least one carbon ring atom (which may optionally be oxidized) in the corresponding heteroatom-containing ring. Unless defined otherwise, “heterocyclylene” preferably refers to heteroarylene, heterocycloalkylene or heterocycloalkenylene.

As used herein, the term “aryl” refers to an aromatic hydrocarbon ring group, including monocyclic aromatic rings as well as bridged ring and/or fused ring systems containing at least one aromatic ring (e.g., ring systems composed of two or three fused rings, wherein at least one of these fused rings is aromatic; or bridged ring systems composed of two or three rings, wherein at least one of these bridged rings is aromatic). If the aryl is a bridged and/or fused ring system which contains, besides one or more aromatic rings, at least one non-aromatic ring (e.g., a saturated ring or an unsaturated alicyclic ring), then one or more carbon ring atoms in each non-aromatic ring may optionally be oxidized (i.e., to form an oxo group). “Aryl” may, e.g., refer to phenyl, naphthyl, dialinyl (i.e., 1,2-dihydronaphthyl), tetralinyl (i.e., 1,2,3,4-tetrahydronaphthyl), indanyl, indenyl (e.g., 1H-indenyl), anthracenyl, phenanthrenyl, 9H-fluorenyl, or azulenyl. Unless defined otherwise, an “aryl” preferably has 6 to 14 ring atoms, more preferably 6 to 10 ring atoms, even more preferably refers to phenyl or naphthyl, and most preferably refers to phenyl.

As used herein, the term “arylene” refers to an aryl group, as defined herein above, but having two points of attachment, i.e. a divalent aromatic hydrocarbon ring group, including monocyclic aromatic rings as well as bridged ring and/or fused ring systems containing at least one aromatic ring (e.g., ring systems composed of two or three fused rings, wherein at least one of these fused rings is aromatic; or bridged ring systems composed of two or three rings, wherein at least one of these bridged rings is aromatic). If the arylene is a bridged and/or fused ring system which contains, besides one or more aromatic rings, at least one non-aromatic ring (e.g., a saturated ring or an unsaturated alicyclic ring), then one or more carbon ring atoms in each non-aromatic ring may optionally be oxidized (i.e., to form an oxo group). “Arylene” may, e.g., refer to phenylene (e.g., phen-1,2-diyl, phen-1,3-diyl, or phen-1,4-diyl), naphthylene (e.g., naphthalen-1,2-diyl, naphthalen-1,3-diyl, naphthalen-1,4-diyl, naphthalen-1,5-diyl, naphthalen-1,6-diyl, naphthalen-1,7-diyl, naphthalen-2,3-diyl, naphthalen-2,5-diyl, naphthalen-2,6-diyl, naphthalen-2,7-diyl, or naphthalen-2,8-diyl), 1,2-dihydronaphthylene, 1,2,3,4-tetrahydronaphthylene, indanylene, indenylene, anthracenylene, phenanthrenylene, 9H-fluorenylene, or azulenylene. Unless defined otherwise, an “arylene” preferably has 6 to 14 ring atoms, more preferably 6 to 10 ring atoms, even more preferably refers to phenylene or naphthylene, and most preferably refers to phenylene (particularly phen-1,4-diyl).

As used herein, the term “heteroaryl” refers to an aromatic ring group, including monocyclic aromatic rings as well as bridged ring and/or fused ring systems containing at least one aromatic ring (e.g., ring systems composed of two or three fused rings, wherein at least one of these fused rings is aromatic; or bridged ring systems composed of two or three rings, wherein at least one of these bridged rings is aromatic), wherein said aromatic ring group comprises one or more (such as, e.g., one, two, three, or four) ring heteroatoms independently selected from O, S and N, and the remaining ring atoms are carbon atoms, wherein one or more S ring atoms (if present) and/or one or more N ring atoms (if present) may optionally be oxidized, and further wherein one or more carbon ring atoms may optionally be oxidized (i.e., to form an oxo group). For example, each heteroatom-containing ring comprised in said aromatic ring group may contain one or two 0 atoms and/or one or two S atoms (which may optionally be oxidized) and/or one, two, three or four N atoms (which may optionally be oxidized), provided that the total number of heteroatoms in the corresponding heteroatom-containing ring is 1 to 4 and that there is at least one carbon ring atom (which may optionally be oxidized) in the corresponding heteroatom-containing ring. “Heteroaryl” may, e.g., refer to thienyl (i.e., thiophenyl), benzo[b]thienyl, naphtho[2,3-b]thienyl, thianthrenyl, furyl (i.e., furanyl), benzofuranyl, isobenzofuranyl, chromanyl, chromenyl (e.g., 2H-1-benzopyranyl or 4H-1-benzopyranyl), isochromenyl (e.g., 1H-2-benzopyranyl), chromonyl, xanthenyl, phenoxathiinyl, pyrrolyl (e.g., 1H-pyrrolyl), imidazolyl, pyrazolyl, pyridyl (i.e., pyridinyl; e.g., 2-pyridyl, 3-pyridyl, or 4-pyridyl), pyrazinyl, pyrimidinyl, pyridazinyl, indolyl (e.g., 3H-indolyl), isoindolyl, indazolyl, indolizinyl, purinyl, quinolyl, isoquinolyl, phthalazinyl, naphthyridinyl, quinoxalinyl, cinnolinyl, pteridinyl, carbazolyl, β-carbolinyl, phenanthridinyl, acridinyl, perimidinyl, phenanthrolinyl (e.g., [1,10]phenanthrolinyl, [1,7]phenanthrolinyl, or [4,7]phenanthrolinyl), phenazinyl, thiazolyl, isothiazolyl, phenothiazinyl, oxazolyl, isoxazolyl, oxadiazolyl (e.g., 1,2,4-oxadiazolyl, 1,2,5-oxadiazolyl (i.e., furazanyl), or 1,3,4-oxadiazolyl), thiadiazolyl (e.g., 1,2,4-thiadiazolyl, 1,2,5-thiadiazolyl, or 1,3,4-thiadiazolyl), phenoxazinyl, pyrazolo[1,5-a]pyrimidinyl (e.g., pyrazolo[1,5-a]pyrimidin-3-yl), 1,2-benzoisoxazol-3-yl, benzothiazolyl, benzothiadiazolyl, benzoxazolyl, benzisoxazolyl, benzimidazolyl, benzo[b]thiophenyl (i.e., benzothienyl), triazolyl (e.g., 1H-1,2,3-triazolyl, 2H-1,2,3-triazolyl, 1H-1,2,4-triazolyl, or 4H-1,2,4-triazolyl), benzotriazolyl, 1H-tetrazolyl, 2H-tetrazolyl, triazinyl (e.g., 1,2,3-triazinyl, 1,2,4-triazinyl, or 1,3,5-triazinyl), furo[2,3-c]pyridinyl, dihydrofuropyridinyl (e.g., 2,3-dihydrofuro[2,3-c]pyridinyl or 1,3-dihydrofuro[3,4-c]pyridinyl), imidazopyridinyl (e.g., imidazo[1,2-a]pyridinyl or imidazo[3,2-a]pyridinyl), quinazolinyl, thienopyridinyl, tetrahydrothienopyridinyl (e.g., 4,5,6,7-tetrahydrothieno[3,2-c]pyridinyl), dibenzofuranyl, 1,3-benzodioxolyl, benzodioxanyl (e.g., 1,3-benzodioxanyl or 1,4-benzodioxanyl), or coumarinyl. Unless defined otherwise, the term “heteroaryl” preferably refers to a 5 to 14 membered (more preferably 5 to 10 membered) monocyclic ring or fused ring system comprising one or more (e.g., one, two, three or four) ring heteroatoms independently selected from O, S and N, wherein one or more S ring atoms (if present) and/or one or more N ring atoms (if present) are optionally oxidized, and wherein one or more carbon ring atoms are optionally oxidized; even more preferably, a “heteroaryl” refers to a 5 or 6 membered monocyclic ring comprising one or more (e.g., one, two or three) ring heteroatoms independently selected from O, S and N, wherein one or more S ring atoms (if present) and/or one or more N ring atoms (if present) are optionally oxidized, and wherein one or more carbon ring atoms are optionally oxidized. Moreover, unless defined otherwise, particularly preferred examples of a “heteroaryl” include pyridinyl (e.g., 2-pyridyl, 3-pyridyl, or 4-pyridyl), imidazolyl, thiazolyl, 1H-tetrazolyl, 2H-tetrazolyl, thienyl (i.e., thiophenyl), or pyrimidinyl.

As used herein, the term “heteroarylene” refers to a heteroaryl group, as defined herein above, but having two points of attachment, i.e. a divalent aromatic ring group, including monocyclic aromatic rings as well as bridged ring and/or fused ring systems containing at least one aromatic ring (e.g., ring systems composed of two or three fused rings, wherein at least one of these fused rings is aromatic; or bridged ring systems composed of two or three rings, wherein at least one of these bridged rings is aromatic), wherein said aromatic ring group comprises one or more (such as, e.g., one, two, three, or four) ring heteroatoms independently selected from O, S and N, and the remaining ring atoms are carbon atoms, wherein one or more S ring atoms (if present) and/or one or more N ring atoms (if present) may optionally be oxidized, and further wherein one or more carbon ring atoms may optionally be oxidized (i.e., to form an oxo group). For example, each heteroatom-containing ring comprised in said aromatic ring group may contain one or two O atoms and/or one or two S atoms (which may optionally be oxidized) and/or one, two, three, or four N atoms (which may optionally be oxidized), provided that the total number of heteroatoms in the corresponding heteroatom-containing ring is 1 to 4 and that there is at least one carbon ring atom (which may optionally be oxidized) in the corresponding heteroatom-containing ring. “Heteroarylene” may, e.g., refer to thienylene (i.e., thiophenylene; e.g., thien-2,3-diyl, thien-2,4-diyl, or thien-2,5-diyl), benzo[b]thienylene, naphtho[2,3-b]thienylene, thianthrenylene, furylene (i.e., furanylene; e.g., furan-2,3-diyl, furan-2,4-diyl, or furan-2,5-diyl), benzofuranylene, isobenzofuranylene, chromanylene, chromenylene, isochromenylene, chromonylene, xanthenylene, phenoxathiinylene, pyrrolylene, imidazolylene, pyrazolylene, pyridylene (i.e., pyridinylene), pyrazinylene, pyrimidinylene, pyridazinylene, indolylene, isoindolylene, indazolylene, indolizinylene, purinylene, quinolylene, isoquinolylene, phthalazinylene, naphthyridinylene, quinoxalinylene, cinnolinylene, pteridinylene, carbazolylene, β-carbolinylene, phenanthridinylene, acridinylene, perimidinylene, phenanthrolinylene, phenazinylene, thiazolylene (e.g., thiazol-2,4-diyl, thiazol-2,5-diyl, or thiazol-4,5-diyl), isothiazolylene (e.g., isothiazol-3,4-diyl, isothiazol-3,5-diyl, or isothiazol-4,5-diyl), phenothiazinylene, oxazolylene (e.g., oxazol-2,4-diyl, oxazol-2,5-diyl, or oxazol-4,5-diyl), isoxazolylene (e.g., isoxazol-3,4-diyl, isoxazol-3,5-diyl, or isoxazol-4,5-diyl), oxadiazolylene (e.g., 1,2,4-oxadiazol-3,5-diyl, 1,2,5-oxadiazol-3,4-diyl, or 1,3,4-oxadiazol-2,5-diyl), thiadiazolylene (e.g., 1,2,4-thiadiazol-3,5-diyl, 1,2,5-thiadiazol-3,4-diyl, or 1,3,4-thiadiazol-2,5-diyl), phenoxazinylene, pyrazolo[1,5-a]pyrimidinylene, 1,2-benzoisoxazolylene, benzothiazolylene, benzothiadiazolylene, benzoxazolylene, benzisoxazolylene, benzimidazolylene, benzo[b]thiophenylene (i.e., benzothienylene), triazolylene (e.g., 1H-1,2,3-triazolylene, 2H-1,2,3-triazolylene, 1H-1,2,4-triazolylene, or 4H-1,2,4-triazolylene), benzotriazolylene, 1H-tetrazolylene, 2H-tetrazolylene, triazinylene (e.g., 1,2,3-triazinylene, 1,2,4-triazinylene, or 1,3,5-triazinylene), furo[2,3-c]pyridinylene, dihydrofuropyridinylene (e.g., 2,3-dihydrofuro[2,3-c]pyridinylene or 1,3-dihydrofuro[3,4-c]pyridinylene), imidazopyridinylene (e.g., imidazo[1,2-a]pyridinylene or imidazo[3,2-a]pyridinylene), quinazolinylene, thienopyridinylene, tetrahydrothienopyridinylene (e.g., 4,5,6,7-tetrahydrothieno[3,2-c]pyridinylene), dibenzofuranylene, 1,3-benzodioxolylene, benzodioxanylene (e.g., 1,3-benzodioxanylene or 1,4-benzodioxanylene), or coumarinylene. Unless defined otherwise, the term “heteroarylene” preferably refers to a divalent 5 to 14 membered (more preferably 5 to 10 membered) monocyclic ring or fused ring system comprising one or more (e.g., one, two, three or four) ring heteroatoms independently selected from O, S and N, wherein one or more S ring atoms (if present) and/or one or more N ring atoms (if present) are optionally oxidized, and wherein one or more carbon ring atoms are optionally oxidized; even more preferably, a “heteroarylene” refers to a divalent 5 or 6 membered monocyclic ring comprising one or more (e.g., one, two or three) ring heteroatoms independently selected from O, S, and N, wherein one or more S ring atoms (if present) and/or one or more N ring atoms (if present) are optionally oxidized, and wherein one or more carbon ring atoms are optionally oxidized. A “heteroarylene”, including any of the specific heteroarylene groups described herein, may be attached through two carbon ring atoms, particularly through those two carbon ring atoms that have the greatest distance from one another (in terms of the number of ring atoms separating them by the shortest possible connection) within one single ring or within the entire ring system of the corresponding heteroarylene. Moreover, unless defined otherwise, particularly preferred examples of a “heteroarylene” include pyridinylene, imidazolylene, thiazolylene, 1H-tetrazolylene, 2H-tetrazolylene, thienylene (i.e., thiophenylene), or pyrimidinylene.

As used herein, the term “cycloalkyl” refers to a saturated hydrocarbon ring group, including monocyclic rings as well as bridged ring, spiro ring and/or fused ring systems (which may be composed, e.g., of two or three rings; such as, e.g., a fused ring system composed of two or three fused rings). “Cycloalkyl” may, e.g., refer to cyclopropyl, cyclobutyl, cyclopentyl, cyclohexyl, cycloheptyl, decalinyl (i.e., decahydronaphthyl), or adamantyl. Unless defined otherwise, “cycloalkyl” preferably refers to a C₃₋₁₁ cycloalkyl, and more preferably refers to a C₃₋₇ cycloalkyl. A particularly preferred “cycloalkyl” is a monocyclic saturated hydrocarbon ring having 3 to 7 ring members. Moreover, unless defined otherwise, particularly preferred examples of a “cycloalkyl” include cyclohexyl or cyclopropyl, particularly cyclohexyl.

As used herein, the term “cycloalkylene” refers to a cycloalkyl group, as defined herein above, but having two points of attachment, i.e. a divalent saturated hydrocarbon ring group. “Cycloalkylene” may, e.g., refer to cyclopropylene (e.g., cyclopropan-1,1-diyl or cyclopropan-1,2-diyl), cyclobutylene (e.g., cyclobutan-1,1-diyl, cyclobutan-1,2-diyl, or cyclobutan-1,3-diyl), cyclopentylene (e.g., cyclopentan-1,1-diyl, cyclopentan-1,2-diyl, or cyclopentan-1,3-diyl), or cyclohexylene (e.g., cyclohexan-1,1-diyl, cyclohexan-1,2-diyl, cyclohexan-1,3-diyl, or cyclohexan-1,4-diyl). Unless defined otherwise, “cycloalkylene” preferably refers to a C₃₋₇ cycloalkylene, and more preferably refers to a C₃₋₅ cycloalkylene. Moreover, unless defined otherwise, a particularly preferred example of a “cycloalkylene” is cyclopropylene.

As used herein, the term “heterocycloalkyl” refers to a saturated ring group, including monocyclic rings as well as bridged ring, spiro ring and/or fused ring systems (which may be composed, e.g., of two or three rings; such as, e.g., a fused ring system composed of two or three fused rings), wherein said ring group contains one or more (such as, e.g., one, two, three, or four) ring heteroatoms independently selected from O, S and N, and the remaining ring atoms are carbon atoms, wherein one or more S ring atoms (if present) and/or one or more N ring atoms (if present) may optionally be oxidized, and further wherein one or more carbon ring atoms may optionally be oxidized (i.e., to form an oxo group). For example, each heteroatom-containing ring comprised in said saturated ring group may contain one or two O atoms and/or one or two S atoms (which may optionally be oxidized) and/or one, two, three or four N atoms (which may optionally be oxidized), provided that the total number of heteroatoms in the corresponding heteroatom-containing ring is 1 to 4 and that there is at least one carbon ring atom (which may optionally be oxidized) in the corresponding heteroatom-containing ring. “Heterocycloalkyl” may, e.g., refer to aziridinyl, azetidinyl, pyrrolidinyl, imidazolidinyl, pyrazolidinyl, piperidinyl, piperazinyl, azepanyl, diazepanyl (e.g., 1,4-diazepanyl), oxazolidinyl, isoxazolidinyl, thiazolidinyl, isothiazolidinyl, morpholinyl (e.g., morpholin-4-yl), thiomorpholinyl (e.g., thiomorpholin-4-yl), oxazepanyl, oxiranyl, oxetanyl, tetrahydrofuranyl, 1,3-dioxolanyl, tetrahydropyranyl, 1,4-dioxanyl, oxepanyl, thiiranyl, thietanyl, tetrahydrothiophenyl (i.e., thiolanyl), 1,3-dithiolanyl, thianyl, thiepanyl, decahydroquinolinyl, decahydroisoquinolinyl, or 2-oxa-5-aza-bicyclo[2.2.1]hept-5-yl. Unless defined otherwise, “heterocycloalkyl” preferably refers to a 3 to 11 membered saturated ring group, which is a monocyclic ring or a fused ring system (e.g., a fused ring system composed of two fused rings), wherein said ring group contains one or more (e.g., one, two, three, or four) ring heteroatoms independently selected from O, S and N, wherein one or more S ring atoms (if present) and/or one or more N ring atoms (if present) are optionally oxidized, and wherein one or more carbon ring atoms are optionally oxidized; more preferably, “heterocycloalkyl” refers to a 5 to 7 membered saturated monocyclic ring group containing one or more (e.g., one, two, or three) ring heteroatoms independently selected from O, S and N, wherein one or more S ring atoms (if present) and/or one or more N ring atoms (if present) are optionally oxidized, and wherein one or more carbon ring atoms are optionally oxidized. Moreover, unless defined otherwise, particularly preferred examples of a “heterocycloalkyl” include tetrahydropyranyl, piperidinyl, piperazinyl, morpholinyl, pyrrolidinyl, or tetrahydrofuranyl.

As used herein, the term “heterocycloalkylene” refers to a heterocycloalkyl group, as defined herein above, but having two points of attachment. “Heterocycloalkylene” may, e.g., refer to aziridinylene, azetidinylene, pyrrolidinylene, imidazolidinylene, pyrazolidinylene, piperidinylene, piperazinylene, azepanylene, diazepanylene (e.g., 1,4-diazepanylene), oxazolidinylene, isoxazolidinylene, thiazolidinylene, isothiazolidinylene, morpholinylene, thiomorpholinylene, oxazepanylene, oxiranylene, oxetanylene, tetrahydrofuranylene, 1,3-dioxolanylene, tetrahydropyranylene, 1,4-dioxanylene, oxepanylene, thiiranylene, thietanylene, tetrahydrothiophenylene (i.e., thiolanylene), 1,3-dithiolanylene, thianylene, or thiepanylene. Unless defined otherwise, “heterocycloalkylene” preferably refers to a divalent 3 to 7 membered saturated monocyclic ring group, wherein said ring group contains one or more (e.g., one, two, three, or four) ring heteroatoms independently selected from O, S and N, wherein the remaining ring atoms are carbon atoms, wherein one or more S ring atoms (if present) and/or one or more N ring atoms (if present) are optionally oxidized, and wherein one or more carbon ring atoms are optionally oxidized; more preferably, “heterocycloalkylene” refers to a divalent 3 to 5 membered saturated monocyclic ring group containing one or two (preferably one) ring heteroatoms independently selected from O, S and N, wherein the remaining ring atoms are carbon atoms. Moreover, unless defined otherwise, particularly preferred examples of a “heterocycloalkylene” include aziridinylene, oxiranylene, thiiranylene, azetidinylene (e.g., azetidin-3,3-diyl), oxetanylene (e.g., oxetan-3,3-diyl), thietanylene (e.g., thietan-3,3-diyl), pyrrolidinylene, tetrahydrofuranylene, or tetrahydrothiophenylene.

As used herein, the term “cycloalkenyl” refers to an unsaturated alicyclic (non-aromatic) hydrocarbon ring group, including monocyclic rings as well as bridged ring, spiro ring and/or fused ring systems (which may be composed, e.g., of two or three rings; such as, e.g., a fused ring system composed of two or three fused rings), wherein said hydrocarbon ring group comprises one or more (e.g., one or two) carbon-to-carbon double bonds and does not comprise any carbon-to-carbon triple bond. “Cycloalkenyl” may, e.g., refer to cyclopropenyl, cyclobutenyl, cyclopentenyl, cyclohexenyl, cyclohexadienyl, cycloheptenyl, or cycloheptadienyl. Unless defined otherwise, “cycloalkenyl” preferably refers to a C₃₋₁₁ cycloalkenyl, and more preferably refers to a C₃₋₇ cycloalkenyl. A particularly preferred “cycloalkenyl” is a monocyclic unsaturated alicyclic hydrocarbon ring having 3 to 7 ring members and containing one or more (e.g., one or two; preferably one) carbon-to-carbon double bonds.

As used herein, the term “cycloalkenylene” refers to a cycloalkenyl group, as defined herein above, but having two points of attachment, i.e. a divalent unsaturated alicyclic (i.e., non-aromatic) hydrocarbon ring group, including monocyclic rings as well as bridged ring, spiro ring and/or fused ring systems (which may be composed, e.g., of two or three rings; such as, e.g., a fused ring system composed of two or three fused rings), wherein said hydrocarbon ring group comprises one or more (e.g., one or two) carbon-to-carbon double bonds and does not comprise any carbon-to-carbon triple bond. “Cycloalkenylene” may, e.g., refer to cyclopropenylene, cyclobutenylene, cyclopentenylene, cyclohexenylene, cyclohexadienylene, cycloheptenylene, or cycloheptadienylene. Unless defined otherwise, “cycloalkenylene” preferably refers to a C₃₋₁₁ cycloalkenylene, and more preferably refers to a C₃₋₇ cycloalkenylene. A particularly preferred “cycloalkenylene” is a divalent monocyclic unsaturated alicyclic hydrocarbon ring having 3 to 7 ring members and containing one or more (e.g., one or two; preferably one) carbon-to-carbon double bonds.

As used herein, the term “heterocycloalkenyl” refers to an unsaturated alicyclic (non-aromatic) ring group, including monocyclic rings as well as bridged ring, spiro ring and/or fused ring systems (which may be composed, e.g., of two or three rings; such as, e.g., a fused ring system composed of two or three fused rings), wherein said ring group contains one or more (such as, e.g., one, two, three, or four) ring heteroatoms independently selected from O, S and N, and the remaining ring atoms are carbon atoms, wherein one or more S ring atoms (if present) and/or one or more N ring atoms (if present) may optionally be oxidized, wherein one or more carbon ring atoms may optionally be oxidized (i.e., to form an oxo group), and further wherein said ring group comprises at least one double bond between adjacent ring atoms and does not comprise any triple bond between adjacent ring atoms. For example, each heteroatom-containing ring comprised in said unsaturated alicyclic ring group may contain one or two O atoms and/or one or two S atoms (which may optionally be oxidized) and/or one, two, three or four N atoms (which may optionally be oxidized), provided that the total number of heteroatoms in the corresponding heteroatom-containing ring is 1 to 4 and that there is at least one carbon ring atom (which may optionally be oxidized) in the corresponding heteroatom-containing ring. “Heterocycloalkenyl” may, e.g., refer to imidazolinyl (e.g., 2-imidazolinyl (i.e., 4,5-dihydro-1H-imidazolyl), 3-imidazolinyl, or 4-imidazolinyl), tetrahydropyridinyl (e.g., 1,2,3,6-tetrahydropyridinyl), dihydropyridinyl (e.g., 1,2-dihydropyridinyl or 2,3-dihydropyridinyl), pyranyl (e.g., 2H-pyranyl or 4H-pyranyl), thiopyranyl (e.g., 2H-thiopyranyl or 4H-thiopyranyl), dihydropyranyl, dihydrofuranyl, dihydropyrazolyl, dihydropyrazinyl, dihydroisoindolyl, octahydroquinolinyl (e.g., 1,2,3,4,4a,5,6,7-octahydroquinolinyl), or octahydroisoquinolinyl (e.g., 1,2,3,4,5,6,7,8-octahydroisoquinolinyl). Unless defined otherwise, “heterocycloalkenyl” preferably refers to a 3 to 11 membered unsaturated alicyclic ring group, which is a monocyclic ring or a fused ring system (e.g., a fused ring system composed of two fused rings), wherein said ring group contains one or more (e.g., one, two, three, or four) ring heteroatoms independently selected from O, S and N, wherein one or more S ring atoms (if present) and/or one or more N ring atoms (if present) are optionally oxidized, wherein one or more carbon ring atoms are optionally oxidized, and wherein said ring group comprises at least one double bond between adjacent ring atoms and does not comprise any triple bond between adjacent ring atoms; more preferably, “heterocycloalkenyl” refers to a 5 to 7 membered monocyclic unsaturated non-aromatic ring group containing one or more (e.g., one, two, or three) ring heteroatoms independently selected from O, S and N, wherein one or more S ring atoms (if present) and/or one or more N ring atoms (if present) are optionally oxidized, wherein one or more carbon ring atoms are optionally oxidized, and wherein said ring group comprises at least one double bond between adjacent ring atoms and does not comprise any triple bond between adjacent ring atoms.

As used herein, the term “heterocycloalkenylene” refers to a heterocycloalkenyl group, as defined herein above, but having two points of attachment, i.e. a divalent unsaturated alicyclic (i.e., non-aromatic) ring group, including monocyclic rings as well as bridged ring, spiro ring and/or fused ring systems (which may be composed, e.g., of two or three rings; such as, e.g., a fused ring system composed of two or three fused rings), wherein said ring group contains one or more (such as, e.g., one, two, three, or four) ring heteroatoms independently selected from O, S and N, and the remaining ring atoms are carbon atoms, wherein one or more S ring atoms (if present) and/or one or more N ring atoms (if present) may optionally be oxidized, wherein one or more carbon ring atoms may optionally be oxidized (i.e., to form an oxo group), and further wherein said ring group comprises at least one double bond between adjacent ring atoms and does not comprise any triple bond between adjacent ring atoms. For example, each heteroatom-containing ring comprised in said unsaturated alicyclic ring group may contain one or two O atoms and/or one or two S atoms (which may optionally be oxidized) and/or one, two, three or four N atoms (which may optionally be oxidized), provided that the total number of heteroatoms in the corresponding heteroatom-containing ring is 1 to 4 and that there is at least one carbon ring atom (which may optionally be oxidized) in the corresponding heteroatom-containing ring. “Heterocycloalkenylene” may, e.g., refer to imidazolinylene, tetrahydropyridinylene, dihydropyridinylene, pyranylene, thiopyranylene, dihydropyranylene, dihydrofuranylene, dihydropyrazolylene, dihydropyrazinylene, dihydroisoindolylene, octahydroquinolinylene, or octahydroisoquinolinylene. Unless defined otherwise, “heterocycloalkenylene” preferably refers to a divalent 3 to 11 membered unsaturated alicyclic ring group, which is a monocyclic ring or a fused ring system (e.g., a fused ring system composed of two fused rings), wherein said ring group contains one or more (e.g., one, two, three, or four) ring heteroatoms independently selected from O, S and N, wherein one or more S ring atoms (if present) and/or one or more N ring atoms (if present) are optionally oxidized, wherein one or more carbon ring atoms are optionally oxidized, and wherein said ring group comprises at least one double bond between adjacent ring atoms and does not comprise any triple bond between adjacent ring atoms; more preferably, “heterocycloalkenylene” refers to a divalent 5 to 7 membered monocyclic unsaturated non-aromatic ring group containing one or more (e.g., one, two, or three) ring heteroatoms independently selected from O, S and N, wherein one or more S ring atoms (if present) and/or one or more N ring atoms (if present) are optionally oxidized, wherein one or more carbon ring atoms are optionally oxidized, and wherein said ring group comprises at least one double bond between adjacent ring atoms and does not comprise any triple bond between adjacent ring atoms.

As used herein, the term “phen-1,2-diyl” refers to a divalent phenyl group which is attached via its 1-position and its 2-position, i.e., to a group having the following formula:

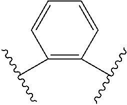 [SMILES: *c1ccccc1*]

As used herein, the term “halogen” refers to fluoro (—F), chloro (—Cl), bromo (—Br), or iodo (—I).

As used herein, the term “haloalkyl” refers to an alkyl group substituted with one or more (preferably 1 to 6, more preferably 1 to 3) halogen atoms which are selected independently from fluoro, chloro, bromo and iodo, and are preferably all fluoro atoms. It will be understood that the maximum number of halogen atoms is limited by the number of available attachment sites and, thus, depends on the number of carbon atoms comprised in the alkyl moiety of the haloalkyl group. “Haloalkyl” may, e.g., refer to —CF₃, —CHF₂, —CH₂F, —CF₂—CH₃, —CH₂—CF₃, —CH₂—CHF₂, —CH₂—CF₂—CH₃, —CH₂—CF₂—CF₃, or —CH(CF₃)₂. A particularly preferred “haloalkyl” group is —CF₃.

The terms “bond” and “covalent bond” are used herein synonymously, unless explicitly indicated otherwise or contradicted by context.

As used herein, the terms “optional”, “optionally” and “may” denote that the indicated feature may be present but can also be absent. Whenever the term “optional”, “optionally” or “may” is used, the present invention specifically relates to both possibilities, i.e., that the corresponding feature is present or, alternatively, that the corresponding feature is absent. For example, the expression “X is optionally substituted with Y” (or “X may be substituted with Y”) means that X is either substituted with Y or is unsubstituted. Likewise, if a component of a composition is indicated to be “optional”, the invention specifically relates to both possibilities, i.e., that the corresponding component is present (contained in the composition) or that the corresponding component is absent from the composition.

Various groups are referred to as being “optionally substituted” in this specification. Generally, these groups may carry one or more substituents, such as, e.g., one, two, three or four substituents. It will be understood that the maximum number of substituents is limited by the number of attachment sites available on the substituted moiety.

Unless defined otherwise, the “optionally substituted” groups referred to in this specification carry preferably not more than two substituents and may, in particular, carry only one substituent. Moreover, unless defined otherwise, it is preferred that the optional substituents are absent, i.e. that the corresponding groups are unsubstituted.

A skilled person will appreciate that the substituent groups comprised in the compounds of the present invention may be attached to the remainder of the respective compound via a number of different positions of the corresponding specific substituent group. Unless defined otherwise, preferred attachment positions for the various specific substituent groups are as illustrated in the examples.

As used herein, unless explicitly indicated otherwise or contradicted by context, the terms “a”, “an” and “the” are used interchangeably with “one or more” and “at least one”. Thus, for example, a composition comprising “a” compound of formula (I) can be interpreted as referring to a composition comprising “one or more” compounds of formula (I).

It is to be understood that wherever numerical ranges are provided/disclosed herein, all values and subranges encompassed by the respective numerical range are meant to be encompassed within the scope of the invention. Accordingly, the present invention specifically and individually relates to each value that falls within a numerical range disclosed herein, as well as each subrange encompassed by a numerical range disclosed herein.

As used herein, the term “comprising” (or “comprise”, “comprises”, “contain”, “contains”, or “containing”), unless explicitly indicated otherwise or contradicted by context, has the meaning of “containing, inter alia”, i.e., “containing, among further optional elements, . . . ”. In addition thereto, this term also includes the narrower meanings of “consisting essentially of” and “consisting of”. For example, the term “A comprising B and C” has the meaning of “A containing, inter alia, B and C”, wherein A may contain further optional elements (e.g., “A containing B, C and D” would also be encompassed), but this term also includes the meaning of “A consisting essentially of B and C” and the meaning of “A consisting of B and C” (i.e., no other components than B and C are comprised in A).

The scope of the present invention embraces all pharmaceutically acceptable salt forms of the compounds of formula (I) which may be formed, e.g., by protonation of an atom carrying an electron lone pair which is susceptible to protonation, such as an amino group, with an inorganic or organic acid, or as a salt of an acid group (such as a carboxylic acid group) with a physiologically acceptable cation. Exemplary base addition salts comprise, for example: alkali metal salts such as sodium or potassium salts; alkaline earth metal salts such as calcium or magnesium salts; zinc salts; ammonium salts; aliphatic amine salts such as trimethylamine, triethylamine, dicyclohexylamine, ethanolamine, diethanolamine, triethanolamine, procaine salts, meglumine salts, ethylenediamine salts, or choline salts; aralkyl amine salts such as N,N-dibenzylethylenediamine salts, benzathine salts, benethamine salts; heterocyclic aromatic amine salts such as pyridine salts, picoline salts, quinoline salts or isoquinoline salts; quaternary ammonium salts such as tetramethylammonium salts, tetraethylammonium salts, benzyltrimethylammonium salts, benzyltriethylammonium salts, benzyltributylammonium salts, methyltrioctylammonium salts or tetrabutylammonium salts; and basic amino acid salts such as arginine salts, lysine salts, or histidine salts. Exemplary acid addition salts comprise, for example: mineral acid salts such as hydrochloride, hydrobromide, hydroiodide, sulfate salts (such as, e.g., sulfate or hydrogensulfate salts), nitrate salts, phosphate salts (such as, e.g., phosphate, hydrogenphosphate, or dihydrogenphosphate salts), carbonate salts, hydrogencarbonate salts, perchlorate salts, borate salts, or thiocyanate salts; organic acid salts such as acetate, propionate, butyrate, pentanoate, hexanoate, heptanoate, octanoate, cyclopentanepropionate, decanoate, undecanoate, oleate, stearate, lactate, maleate, oxalate, fumarate, tartrate, malate, citrate, succinate, adipate, gluconate, glycolate, nicotinate, benzoate, salicylate, ascorbate, pamoate (embonate), camphorate, glucoheptanoate, or pivalate salts; sulfonate salts such as methanesulfonate (mesylate), ethanesulfonate (esylate), 2-hydroxyethanesulfonate (isethionate), benzenesulfonate (besylate), p-toluenesulfonate (tosylate), 2-naphthalenesulfonate (napsylate), 3-phenylsulfonate, or camphorsulfonate salts; glycerophosphate salts; and acidic amino acid salts such as aspartate or glutamate salts. A pharmaceutically acceptable salt of the compound of formula (I) is preferably not a hydroiodide salt. Preferred pharmaceutically acceptable salts of the compounds of formula (I) include a hydrochloride salt, a hydrobromide salt, a mesylate salt, a sulfate salt, a tartrate salt, a fumarate salt, an acetate salt, an oxalate salt, a citrate salt, and a phosphate salt. A particularly preferred pharmaceutically acceptable salt of the compound of formula (I) is a hydrochloride salt. Accordingly, if a compound of formula (I), including any one of the specific compounds of formula (I) described herein, is provided in the form of a pharmaceutically acceptable salt, it is preferred that the respective compound is in the form of a hydrochloride salt, a hydrobromide salt, a mesylate salt, a sulfate salt, a tartrate salt, a fumarate salt, an acetate salt, an oxalate salt, a citrate salt, or a phosphate salt, and it is particularly preferred that it is in the form of a hydrochloride salt.

The present invention also specifically relates to the compound of formula (I), including any one of the specific compounds of formula (I) described herein, in non-salt form.

Moreover, the scope of the invention embraces the compounds of formula (I) in any solvated form, including, e.g., solvates with water (i.e., as a hydrate) or solvates with organic solvents such as, e.g., methanol, ethanol, isopropanol, acetic acid, ethyl acetate, ethanolamine, DMSO, or acetonitrile. All physical forms, including any amorphous or crystalline forms (i.e., polymorphs), of the compounds of formula (I) are also encompassed within the scope of the invention. It is to be understood that such solvates and physical forms of pharmaceutically acceptable salts of the compounds of the formula (I) are likewise embraced by the invention.

Furthermore, the compounds of formula (I) may exist in the form of different isomers, in particular stereoisomers (including, e.g., geometric isomers (or cis/trans isomers), enantiomers and diastereomers) or tautomers (including, in particular, prototropic tautomers, such as keto/enol tautomers or thione/thiol tautomers). All such isomers of the compounds of formula (I) are contemplated as being part of the present invention, either in admixture or in pure or substantially pure form. As for stereoisomers, the invention embraces the isolated optical isomers of the compounds according to the invention as well as any mixtures thereof (including, in particular, racemic mixtures/racemates). The racemates can be resolved by physical methods, such as, e.g., fractional crystallization, separation or crystallization of diastereomeric derivatives, or separation by chiral column chromatography. The individual optical isomers can also be obtained from the racemates via salt formation with an optically active acid followed by crystallization. The present invention further encompasses any tautomers of the compounds of formula (I). It will be understood that some compounds may exhibit tautomerism. In such cases, the formulae provided herein expressly depict only one of the possible tautomeric forms. The formulae and chemical names as provided herein are intended to encompass any tautomeric form of the corresponding compound and not to be limited merely to the specific tautomeric form depicted by the drawing or identified by the name of the compound.

The scope of the invention also embraces compounds of formula (I), in which one or more atoms are replaced by a specific isotope of the corresponding atom. For example, the invention encompasses compounds of formula (I), in which one or more hydrogen atoms (or, e.g., all hydrogen atoms) are replaced by deuterium atoms (i.e., ²H; also referred to as “D”). Accordingly, the invention also embraces compounds of formula (I) which are enriched in deuterium. Naturally occurring hydrogen is an isotopic mixture comprising about 99.98 mol-% hydrogen-1 (¹H) and about 0.0156 mol-% deuterium (²H or D). The content of deuterium in one or more hydrogen positions in the compounds of formula (I) can be increased using deuteration techniques known in the art. For example, a compound of formula (I) or a reactant or precursor to be used in the synthesis of the compound of formula (I) can be subjected to an H/D exchange reaction using, e.g., heavy water (D₂O). Further suitable deuteration techniques are described in: Atzrodt J et a1., Bioorg Med Chem, 20(18), 5658-5667, 2012; William J S et a1., Journal of Labelled Compounds and Radiopharmaceuticals, 53(11-12), 635-644, 2010; Modvig A et a1., J Org Chem, 79, 5861-5868, 2014. The content of deuterium can be determined, e.g., using mass spectrometry or NMR spectroscopy. Unless specifically indicated otherwise, it is preferred that the compound of formula (I) is not enriched in deuterium. Accordingly, the presence of naturally occurring hydrogen atoms or ¹H hydrogen atoms in the compounds of formula (I) is preferred.

The present invention also embraces compounds of formula (I), in which one or more atoms are replaced by a positron-emitting isotope of the corresponding atom, such as, e.g., ¹⁸F, ¹¹C, ¹³N, ¹⁵O, ⁷⁶Br, ⁷⁷Br, ¹²⁰I and/or ¹²⁴I. Such compounds can be used as tracers, trackers or imaging probes in positron emission tomography (PET). The invention thus includes (i) compounds of formula (I), in which one or more fluorine atoms (or, e.g., all fluorine atoms) are replaced by ¹⁸F atoms, (ii) compounds of formula (I), in which one or more carbon atoms (or, e.g., all carbon atoms) are replaced by ¹¹C atoms, (iii) compounds of formula (I), in which one or more nitrogen atoms (or, e.g., all nitrogen atoms) are replaced by ¹³N atoms, (iv) compounds of formula (I), in which one or more oxygen atoms (or, e.g., all oxygen atoms) are replaced by ¹⁵O atoms, (v) compounds of formula (I), in which one or more bromine atoms (or, e.g., all bromine atoms) are replaced by ⁷⁶Br atoms, (vi) compounds of formula (I), in which one or more bromine atoms (or, e.g., all bromine atoms) are replaced by ⁷⁷Br atoms, (vii) compounds of formula (I), in which one or more iodine atoms (or, e.g., all iodine atoms) are replaced by ¹²⁰I atoms, and (viii) compounds of formula (I), in which one or more iodine atoms (or, e.g., all iodine atoms) are replaced by ¹²⁴I atoms. In general, it is preferred that none of the atoms in the compounds of formula (I) are replaced by specific isotopes.

The compounds of formula (I) may be administered as compounds per se or may be formulated as medicaments. The medicaments/pharmaceutical compositions may optionally comprise one or more pharmaceutically acceptable excipients, such as carriers, diluents, fillers, disintegrants, lubricating agents, binders, colorants, pigments, stabilizers, preservatives, antioxidants, and/or solubility enhancers.

The pharmaceutical compositions may comprise one or more solubility enhancers, such as, e.g., poly(ethylene glycol), including poly(ethylene glycol) having a molecular weight in the range of about 200 to about 5,000 Da (e.g., PEG 200, PEG 300, PEG 400, or PEG 600), ethylene glycol, propylene glycol, glycerol, a non-ionic surfactant, tyloxapol, polysorbate 80, macrogol-15-hydroxystearate (e.g., Kolliphor® HS 15, CAS 70142-34-6), a phospholipid, lecithin, dimyristoyl phosphatidylcholine, dipalmitoyl phosphatidylcholine, distearoyl phosphatidylcholine, a cyclodextrin, α-cyclodextrin, β-cyclodextrin, γ-cyclodextrin, hydroxyethyl-β-cyclodextrin, hydroxypropyl-β-cyclodextrin, hydroxyethyl-γ-cyclodextrin, hydroxypropyl-γ-cyclodextrin, dihydroxypropyl-β-cyclodextrin, sulfobutylether-β-cyclodextrin, sulfobutylether-γ-cyclodextrin, glucosyl-α-cyclodextrin, glucosyl-β-cyclodextrin, diglucosyl-β-cyclodextrin, maltosyl-α-cyclodextrin, maltosyl-β-cyclodextrin, maltosyl-γ-cyclodextrin, maltotriosyl-β-cyclodextrin, maltotriosyl-γ-cyclodextrin, dimaltosyl-R-cyclodextrin, methyl-β-cyclodextrin, a carboxyalkyl thioether, hydroxypropyl methylcellulose, hydroxypropylcellulose, polyvinylpyrrolidone, a vinyl acetate copolymer, vinyl pyrrolidone, sodium lauryl sulfate, dioctyl sodium sulfosuccinate, or any combination thereof.

The pharmaceutical compositions may also comprise one or more preservatives, particularly one or more antimicrobial preservatives, such as, e.g., benzyl alcohol, chlorobutanol, 2-ethoxyethanol, m-cresol, chlorocresol (e.g., 2-chloro-3-methyl-phenol or 4-chloro-3-methyl-phenol), benzalkonium chloride, benzethonium chloride, benzoic acid (or a pharmaceutically acceptable salt thereof), sorbic acid (or a pharmaceutically acceptable salt thereof), chlorhexidine, thimerosal, or any combination thereof.

The pharmaceutical compositions can be formulated by techniques known to the person skilled in the art, such as the techniques published in “Remington: The Science and Practice of Pharmacy”, Pharmaceutical Press, 22^{nd} edition. The pharmaceutical compositions can be formulated as dosage forms for oral, parenteral, such as intramuscular, intravenous, subcutaneous, intradermal, intraarterial, intracardial, rectal, nasal, topical, aerosol or vaginal administration. Dosage forms for oral administration include coated and uncoated tablets, soft gelatin capsules, hard gelatin capsules, lozenges, troches, solutions, emulsions, suspensions, syrups, elixirs, powders and granules for reconstitution, dispersible powders and granules, medicated gums, chewing tablets and effervescent tablets. Dosage forms for parenteral administration include solutions, emulsions, suspensions, dispersions and powders and granules for reconstitution. Emulsions are a preferred dosage form for parenteral administration. Dosage forms for rectal and vaginal administration include suppositories and ovula. Dosage forms for nasal administration can be administered via inhalation and insufflation, for example by a metered inhaler. Dosage forms for topical administration include creams, gels, ointments, salves, patches and transdermal delivery systems.

The compounds of formula (I) or the above described pharmaceutical compositions comprising a compound of formula (I) may be administered to a subject by any convenient route of administration, whether systemically/peripherally or at the site of desired action, including but not limited to one or more of: oral (e.g., as a tablet, capsule, or as an ingestible solution), topical (e.g., transdermal, intranasal, ocular, buccal, and sublingual), parenteral (e.g., using injection techniques or infusion techniques, and including, for example, by injection, e.g., subcutaneous, intradermal, intramuscular, intravenous, intraarterial, intracardiac, intrathecal, intraspinal, intracapsular, subcapsular, intraorbital, intraperitoneal, intratracheal, subcuticular, intraarticular, subarachnoid, or intrasternal by, e.g., implant of a depot, for example, subcutaneously or intramuscularly), pulmonary (e.g., by inhalation or insufflation therapy using, e.g., an aerosol, e.g., through mouth or nose), gastrointestinal, intrauterine, intraocular, subcutaneous, ophthalmic (including intravitreal or intracameral), rectal, or vaginal administration.

If said compounds or pharmaceutical compositions are administered parenterally, then examples of such administration include one or more of: intravenously, intraarterially, intraperitoneally, intrathecally, intraventricularly, intraurethrally, intrasternally, intracardially, intracranially, intramuscularly or subcutaneously administering the compounds or pharmaceutical compositions, and/or by using infusion techniques. For parenteral administration, the compounds are best used in the form of a sterile aqueous solution which may contain other substances, for example, enough salts or glucose to make the solution isotonic with blood. The aqueous solutions should be suitably buffered (preferably to a pH of from 3 to 9), if necessary. The preparation of suitable parenteral formulations under sterile conditions is readily accomplished by standard pharmaceutical techniques well known to those skilled in the art.

Said compounds or pharmaceutical compositions can also be administered orally in the form of tablets, capsules, ovules, elixirs, solutions or suspensions, which may contain flavoring or coloring agents, for immediate-, delayed-, modified-, sustained-, pulsed- or controlled-release applications.

The tablets may contain excipients such as microcrystalline cellulose, lactose, sodium citrate, calcium carbonate, dibasic calcium phosphate and glycine, disintegrants such as starch (preferably corn, potato or tapioca starch), sodium starch glycolate, croscarmellose sodium and certain complex silicates, and granulation binders such as polyvinylpyrrolidone, hydroxypropylmethylcellulose (HPMC), hydroxypropylcellulose (HPC), sucrose, gelatin and acacia. Additionally, lubricating agents such as magnesium stearate, stearic acid, glyceryl behenate and talc may be included. Solid compositions of a similar type may also be employed as fillers in gelatin capsules. Preferred excipients in this regard include lactose, starch, a cellulose, or high molecular weight polyethylene glycols. For aqueous suspensions and/or elixirs, the agent may be combined with various sweetening or flavoring agents, coloring matter or dyes, with emulsifying and/or suspending agents and with diluents such as water, ethanol, propylene glycol and glycerin, and combinations thereof.

For oral administration, the compounds or pharmaceutical compositions are preferably administered by oral ingestion, particularly by swallowing. The compounds or pharmaceutical compositions can thus be administered to pass through the mouth into the gastrointestinal tract, which can also be referred to as “oral-gastrointestinal” administration.

Alternatively, said compounds or pharmaceutical compositions can be administered in the form of a suppository or pessary, or may be applied topically in the form of a gel, hydrogel, lotion, solution, cream, ointment or dusting powder. The compounds of the present invention may also be dermally or transdermally administered, for example, by the use of a skin patch.

Said compounds or pharmaceutical compositions may also be administered by sustained release systems. Suitable examples of sustained-release compositions include semi-permeable polymer matrices in the form of shaped articles, e.g., films, or microcapsules. Sustained-release matrices include, e.g., polylactides, copolymers of L-glutamic acid and gamma-ethyl-L-glutamate, poly(2-hydroxyethyl methacrylate), ethylene vinyl acetate, or poly-D-(−)-3-hydroxybutyric acid. Sustained-release pharmaceutical compositions also include liposomally entrapped compounds. The present invention thus also relates to liposomes containing a compound of the invention.

Said compounds or pharmaceutical compositions may also be administered by the pulmonary route, rectal routes, or the ocular route. For ophthalmic use, they can be formulated as micronized suspensions in isotonic, pH adjusted, sterile saline, or, preferably, as solutions in isotonic, pH adjusted, sterile saline, optionally in combination with a preservative such as a benzalkonium chloride. Alternatively, they may be formulated in an ointment such as petrolatum.

It is also envisaged to prepare dry powder formulations of the compounds of formula (I) for pulmonary administration, particularly inhalation. Such dry powders may be prepared by spray drying under conditions which result in a substantially amorphous glassy or a substantially crystalline bioactive powder. Accordingly, dry powders of the compounds of the present invention can be made according to an emulsification/spray drying process.

For topical application to the skin, said compounds or pharmaceutical compositions can be formulated as a suitable ointment containing the active compound suspended or dissolved in, for example, a mixture with one or more of the following: mineral oil, liquid petrolatum, white petrolatum, propylene glycol, emulsifying wax and water. Alternatively, they can be formulated as a suitable lotion or cream, suspended or dissolved in, for example, a mixture of one or more of the following: mineral oil, sorbitan monostearate, a polyethylene glycol, liquid paraffin, polysorbate 60, cetyl esters wax, 2-octyldodecanol, benzyl alcohol and water.

The present invention thus relates to the compounds or the pharmaceutical compositions provided herein, wherein the corresponding compound or pharmaceutical composition is to be administered by any one of: an oral route; topical route, including by transdermal, intranasal, ocular, buccal, or sublingual route; parenteral route using injection techniques or infusion techniques, including by subcutaneous, intradermal, intramuscular, intravenous, intraarterial, intracardiac, intrathecal, intraspinal, intracapsular, subcapsular, intraorbital, intraperitoneal, intratracheal, subcuticular, intraarticular, subarachnoid, intrasternal, intraventricular, intraurethral, or intracranial route; pulmonary route, including by inhalation or insufflation therapy; gastrointestinal route; intrauterine route; intraocular route; subcutaneous route; ophthalmic route, including by intravitreal, or intracameral route; rectal route; or vaginal route. Particularly preferred routes of administration are oral administration or parenteral administration.

Typically, a physician will determine the actual dosage which will be most suitable for an individual subject. The specific dose level and frequency of dosage for any particular individual subject may be varied and will depend upon a variety of factors including the activity of the specific compound employed, the metabolic stability and length of action of that compound, the age, body weight, general health, sex, diet, mode and time of administration, rate of excretion, drug combination, the severity of the particular condition, and the individual subject undergoing therapy.

A proposed, yet non-limiting dose of the compounds according to the invention for oral administration to a human (of approximately 70 kg body weight) may be 0.05 to 2000 mg, preferably 0.1 mg to 1000 mg, of the active ingredient per unit dose. The unit dose may be administered, e.g., 1 to 3 times per day. The unit dose may also be administered 1 to 7 times per week, e.g., with not more than one administration per day. It will be appreciated that it may be necessary to make routine variations to the dosage depending on the age and weight of the patient/subject as well as the severity of the condition to be treated. The precise dose and also the route of administration will ultimately be at the discretion of the attendant physician or veterinarian.

The compound of formula (I) or a pharmaceutical composition comprising the compound of formula (I) can be administered in monotherapy (e.g., without concomitantly administering any further therapeutic agents, or without concomitantly administering any further therapeutic agents against the same disease that is to be treated or prevented with the compound of formula (I)). However, the compound of formula (I) or a pharmaceutical composition comprising the compound of formula (I) can also be administered in combination with one or more further therapeutic agents, preferably in combination with one or more further therapeutic agents selected from antimalarial agents, steroids, methotrexate, Janus kinase inhibitors, Toll-like receptors inhibitors and interferon inhibitors. If the compound of formula (I) is used in combination with a second therapeutic agent active against the same disease or condition, the dose of each compound may differ from that when the corresponding compound is used alone, in particular, a lower dose of each compound may be used. The combination of the compound of formula (I) with one or more further therapeutic agents may comprise the simultaneous/concomitant administration of the compound of formula (I) and the further therapeutic agent(s) (either in a single pharmaceutical formulation or in separate pharmaceutical formulations), or the sequential/separate administration of the compound of formula (I) and the further therapeutic agent(s). If administration is sequential, either the compound of formula (I) according to the invention or the one or more further therapeutic agents may be administered first. If administration is simultaneous, the one or more further therapeutic agents may be included in the same pharmaceutical formulation as the compound of formula (I), or they may be administered in two or more different (separate) pharmaceutical formulations.

The subject or patient to be treated in accordance with the present invention may be an animal (e.g., a non-human animal). Preferably, the subject/patient is a mammal. More preferably, the subject/patient is a human (e.g., a male human or a female human) or a non-human mammal (such as, e.g., a guinea pig, a hamster, a rat, a mouse, a rabbit, a dog, a cat, a horse, a monkey, an ape, a marmoset, a baboon, a gorilla, a chimpanzee, an orangutan, a gibbon, a sheep, cattle, or a pig). Most preferably, the subject/patient to be treated in accordance with the invention is a human.

The term “treatment” of a disorder or disease, as used herein, is well-known in the art. “Treatment” of a disorder or disease implies that a disorder or disease is suspected or has been diagnosed in a patient/subject. A patient/subject suspected of suffering from a disorder or disease typically shows specific clinical and/or pathological symptoms which a skilled person can easily attribute to a specific pathological condition (i.e., diagnose a disorder or disease).

The “treatment” of a disorder or disease may, for example, lead to a halt in the progression of the disorder or disease (e.g., no deterioration of symptoms) or a delay in the progression of the disorder or disease (in case the halt in progression is of a transient nature only). The “treatment” of a disorder or disease may also lead to a partial response (e.g., amelioration of symptoms) or complete response (e.g., disappearance of symptoms) of the subject/patient suffering from the disorder or disease. Accordingly, the “treatment” of a disorder or disease may also refer to an amelioration of the disorder or disease, which may, e.g., lead to a halt in the progression of the disorder or disease or a delay in the progression of the disorder or disease. Such a partial or complete response may be followed by a relapse. It is to be understood that a subject/patient may experience a broad range of responses to a treatment (such as the exemplary responses as described herein above). The treatment of a disorder or disease may, inter alia, comprise curative treatment (preferably leading to a complete response and eventually to healing of the disorder or disease) and palliative treatment (including symptomatic relief).

The term “prevention” of a disorder or disease, as used herein, is also well-known in the art. For example, a patient/subject suspected of being prone to suffer from a disorder or disease may particularly benefit from a prevention of the disorder or disease. The subject/patient may have a susceptibility or predisposition for a disorder or disease, including but not limited to hereditary predisposition. Such a predisposition can be determined by standard methods or assays, using, e.g., genetic markers or phenotypic indicators. It is to be understood that a disorder or disease to be prevented in accordance with the present invention has not been diagnosed or cannot be diagnosed in the patient/subject (for example, the patient/subject does not show any clinical or pathological symptoms). Thus, the term “prevention” comprises the use of a compound of the present invention before any clinical and/or pathological symptoms are diagnosed or determined or can be diagnosed or determined by the attending physician.

It is to be understood that the present invention specifically relates to each and every combination of features described herein, including any combination of general and/or preferred features. In particular, the invention specifically relates to each combination of meanings (including general and/or preferred meanings) for the various groups and variables comprised in formula (I).

In this specification, a number of documents including patent applications, scientific literature and manufacturers' manuals are cited. The disclosure of these documents, while not considered relevant for the patentability of this invention, is herewith incorporated by reference in its entirety. More specifically, all referenced documents are incorporated by reference to the same extent as if each individual document was specifically and individually indicated to be incorporated by reference.

The reference in this specification to any prior publication (or information derived therefrom) is not and should not be taken as an acknowledgment or admission or any form of suggestion that the corresponding prior publication (or the information derived therefrom) forms part of the common general knowledge in the technical field to which the present specification relates.

The present invention is further illustrated by the appended figures which show:

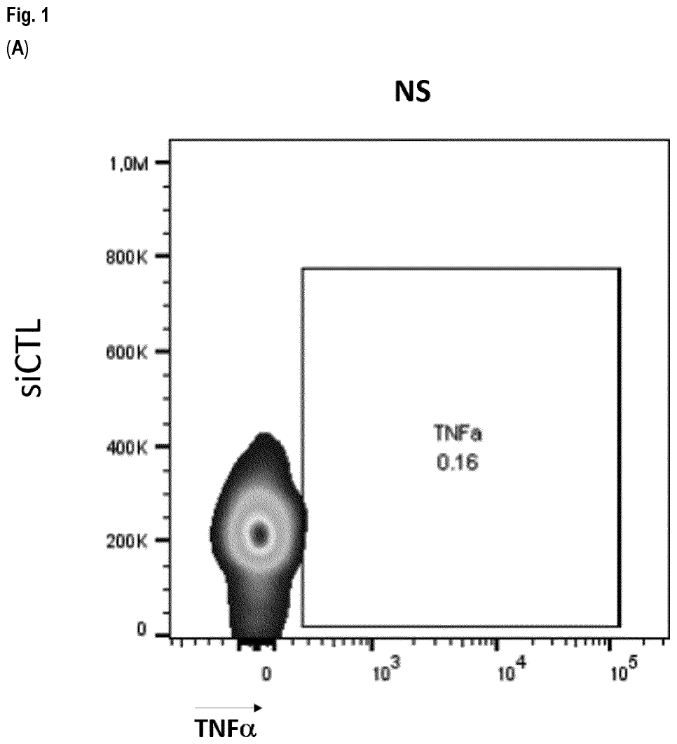

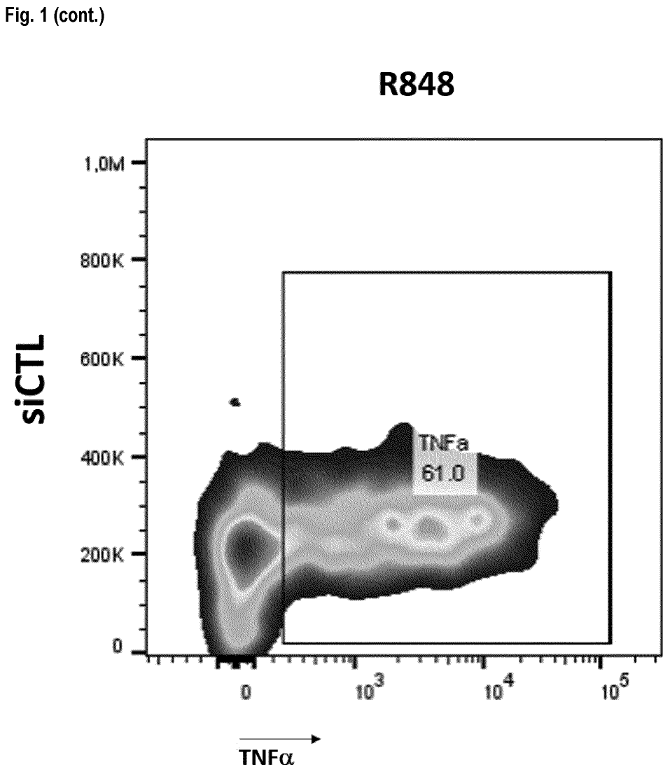

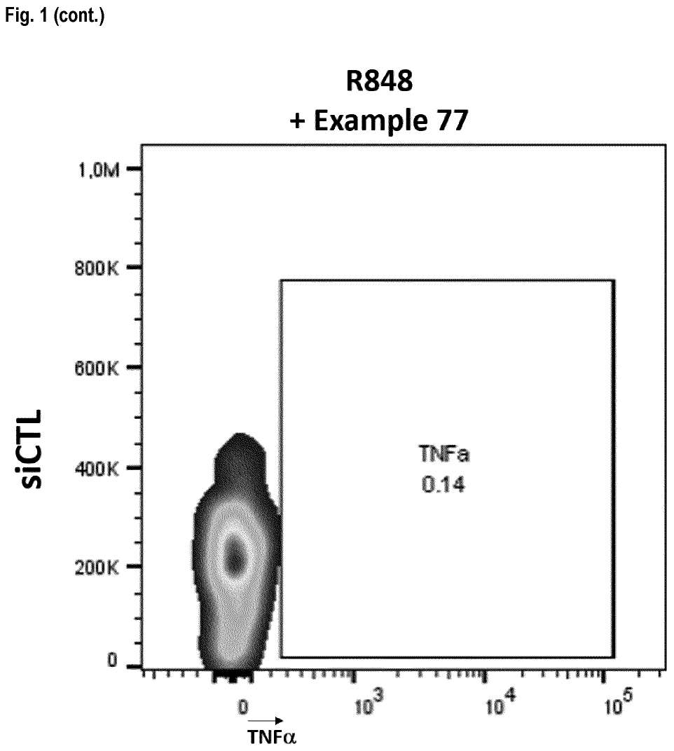

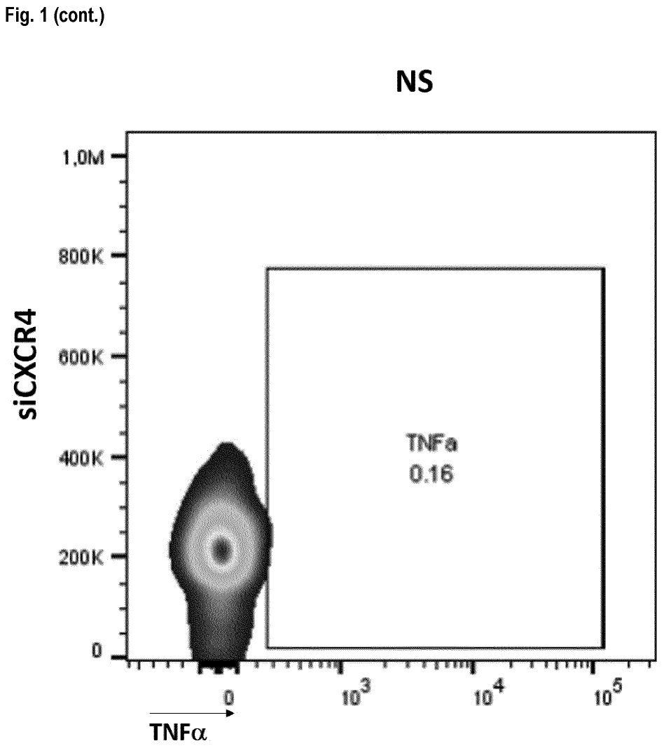

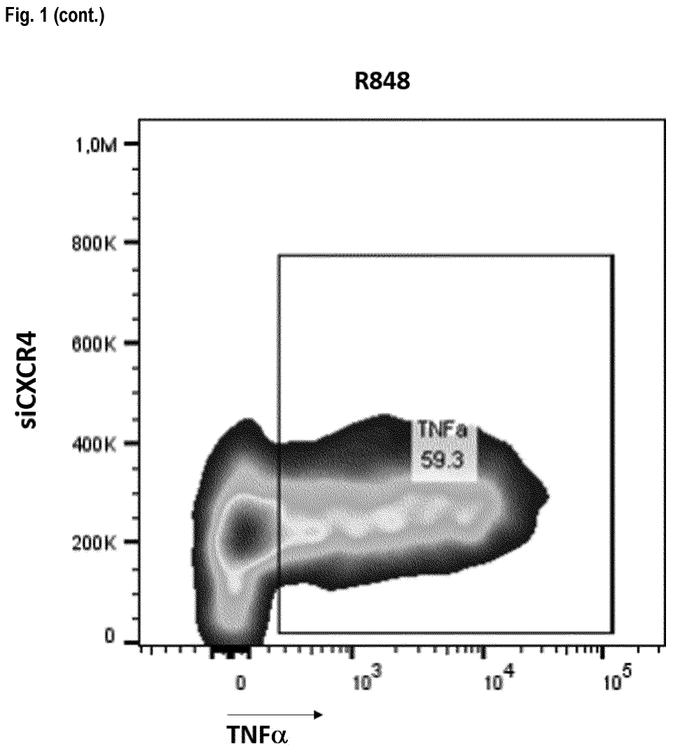

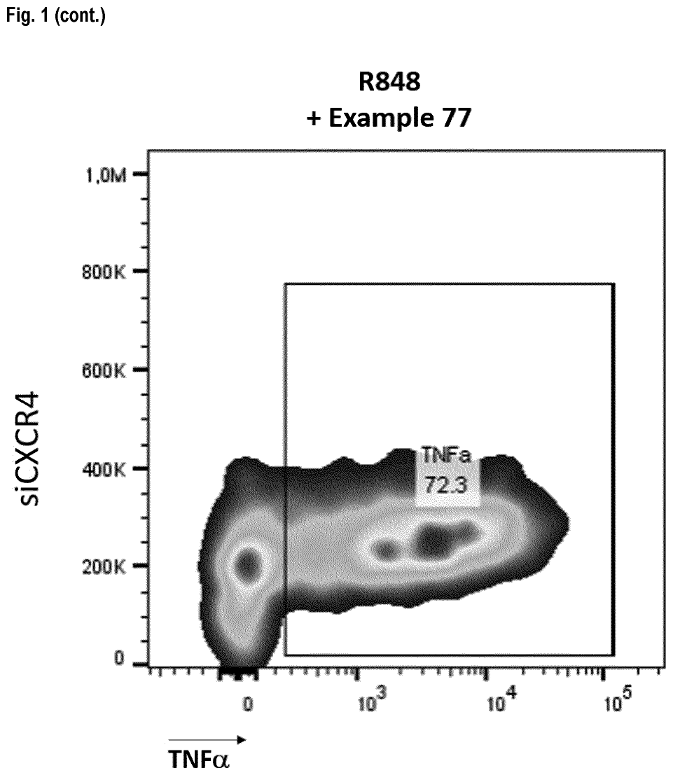

FIG. 1: The anti-inflammatory properties of an exemplary compound of formula (I), i.e. Example 77, are dependent on the chemokine receptor CXCR4. The expression of the CXCR4 receptor was repressed by a specific siRNA (siCXCR4) in healthy donor monocytes. A control siRNA (siCTL) was used as a negative control for the experiment. The monocytes were then treated with Example 77 at 50 nM and activated with R848. Intracellular TNFα production was measured by flow cytometry. (A) Dot plot representation. (B) Histogram representation of the % of TNFα positive cells. See Example 202.

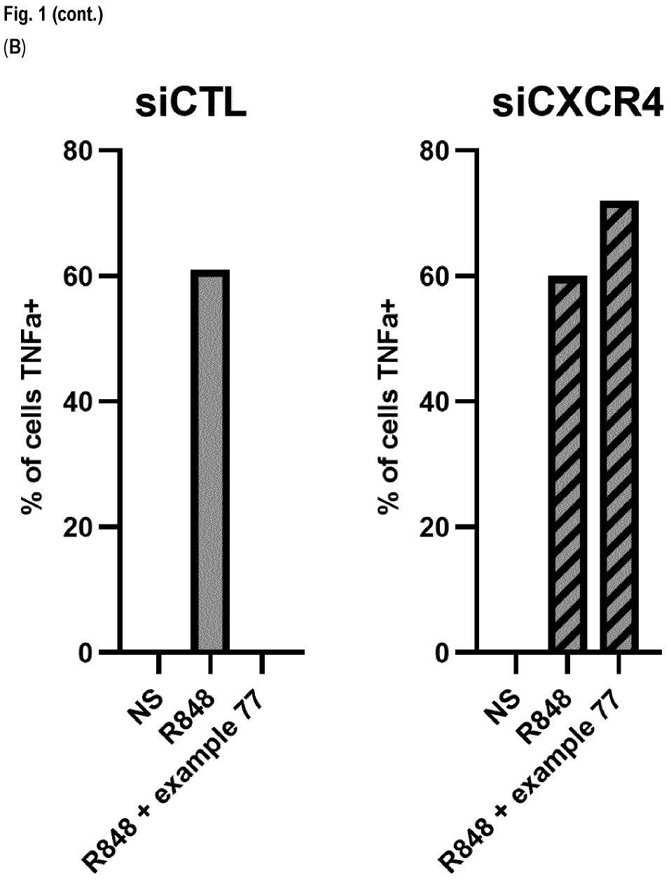

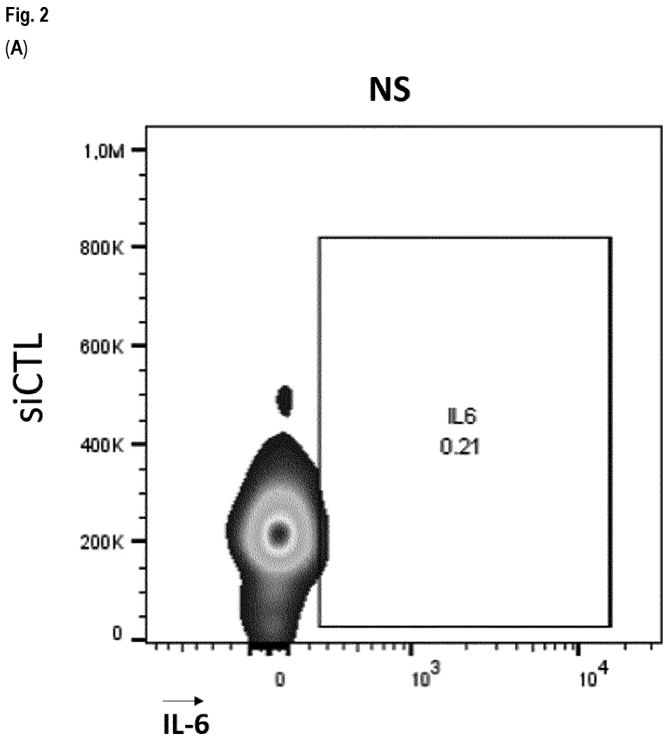

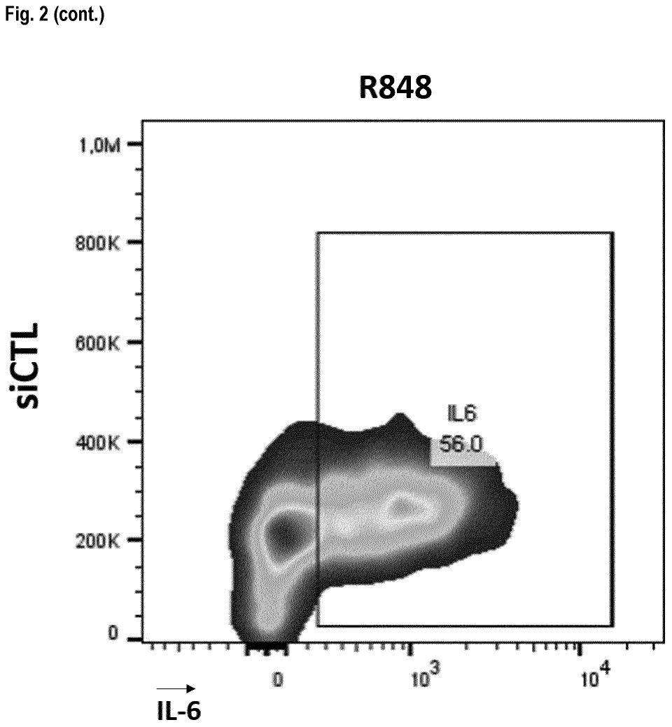

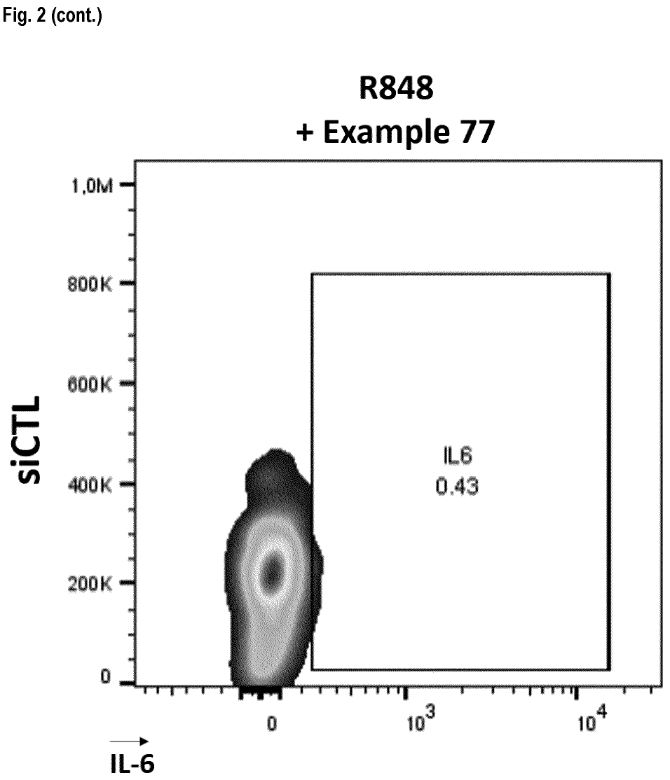

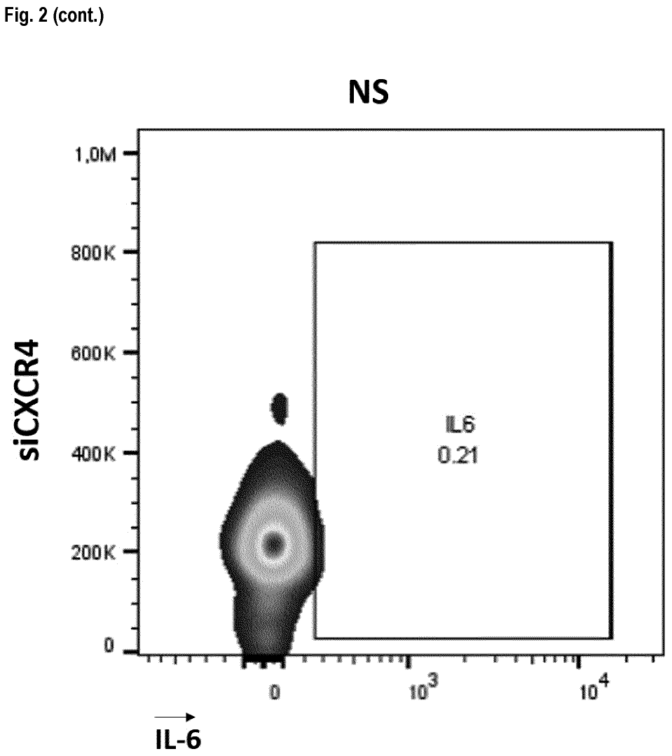

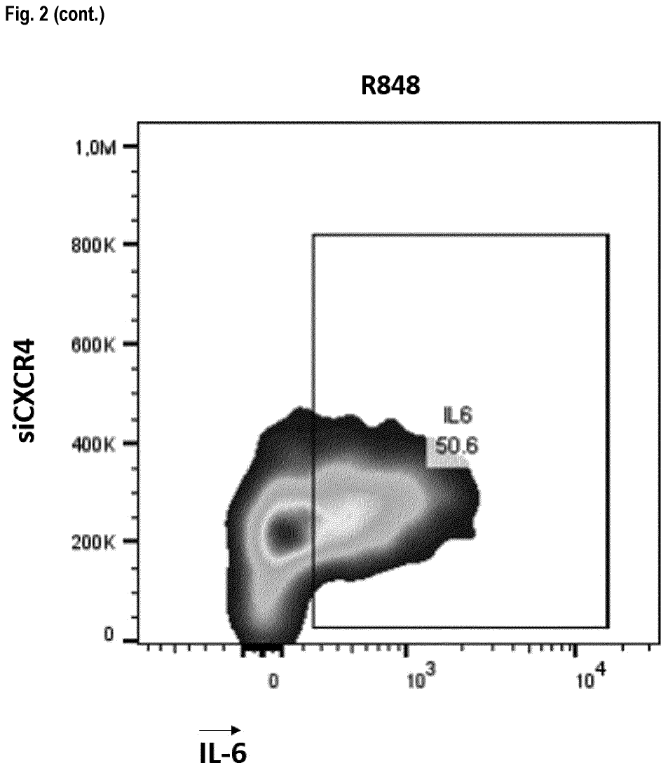

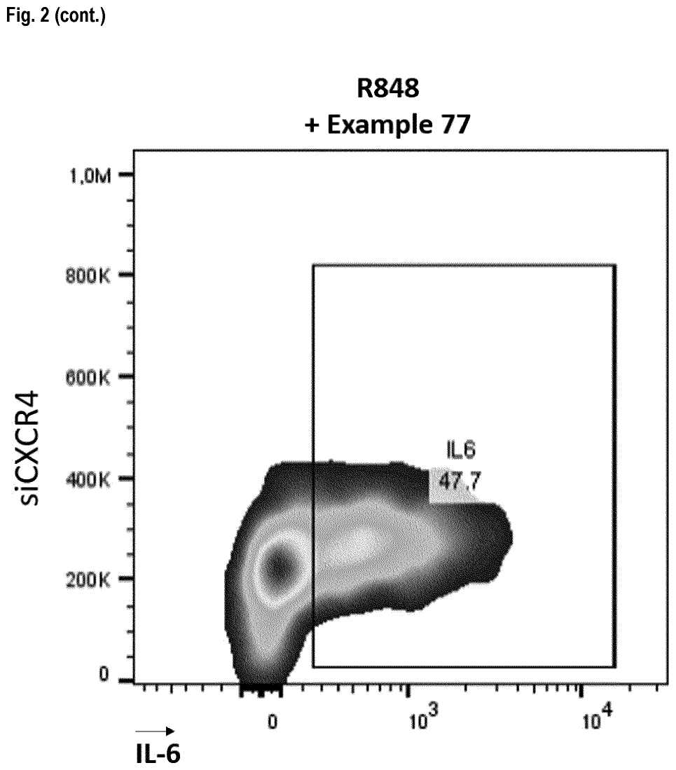

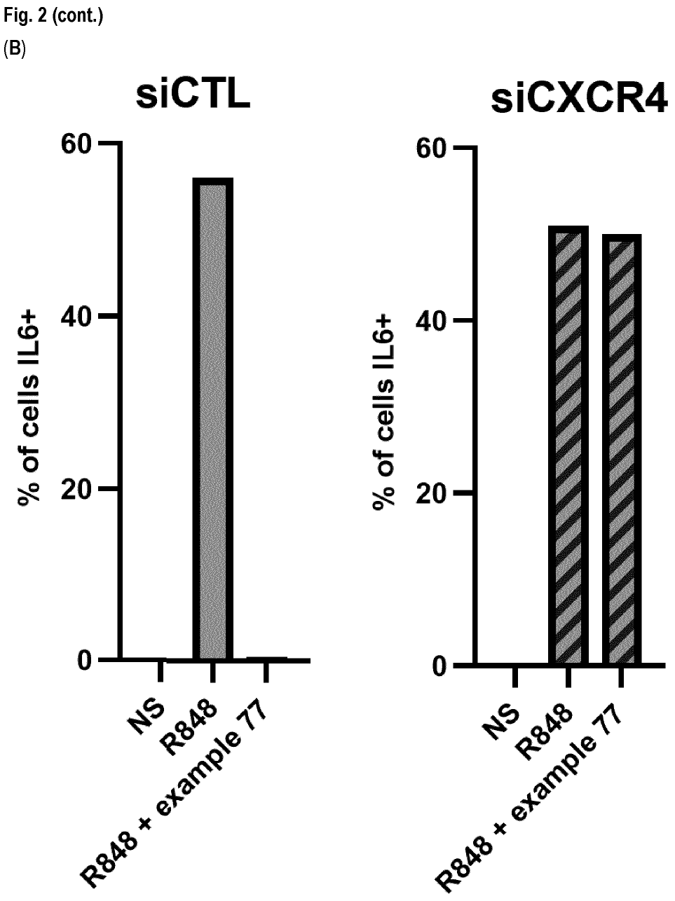

FIG. 2: The anti-inflammatory properties of an exemplary compound of formula (I), i.e. Example 77, are dependent on the chemokine receptor CXCR4. The expression of the CXCR4 receptor was repressed by a specific siRNA (siCXCR4) in healthy donor monocytes. A control siRNA (siCTL) was used as a negative control for the experiment. The monocytes were then treated with Example 77 at 50 nM and activated with R848. Intracellular IL-6 production was measured by flow cytometry. (A) Dot plot representation. (B) Histogram representation of the % of IL-6 positive cells. See Example 202.

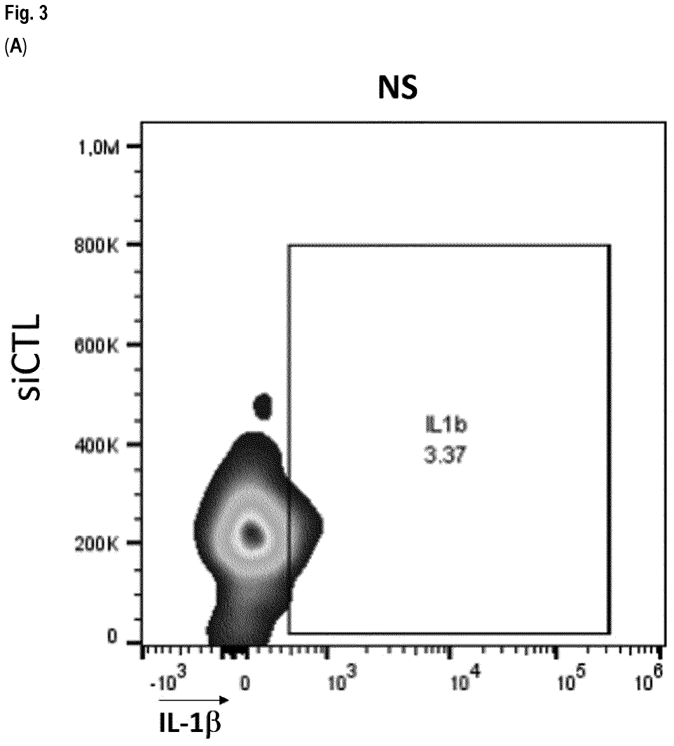

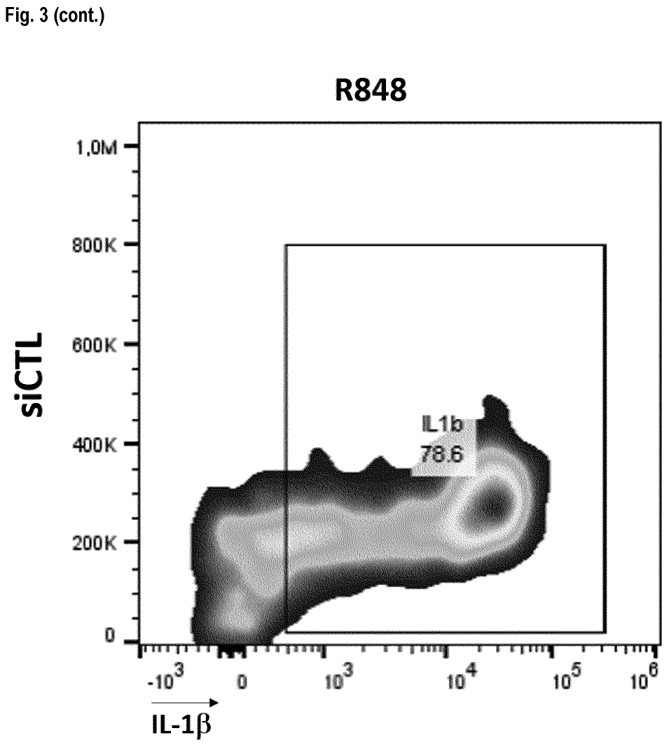

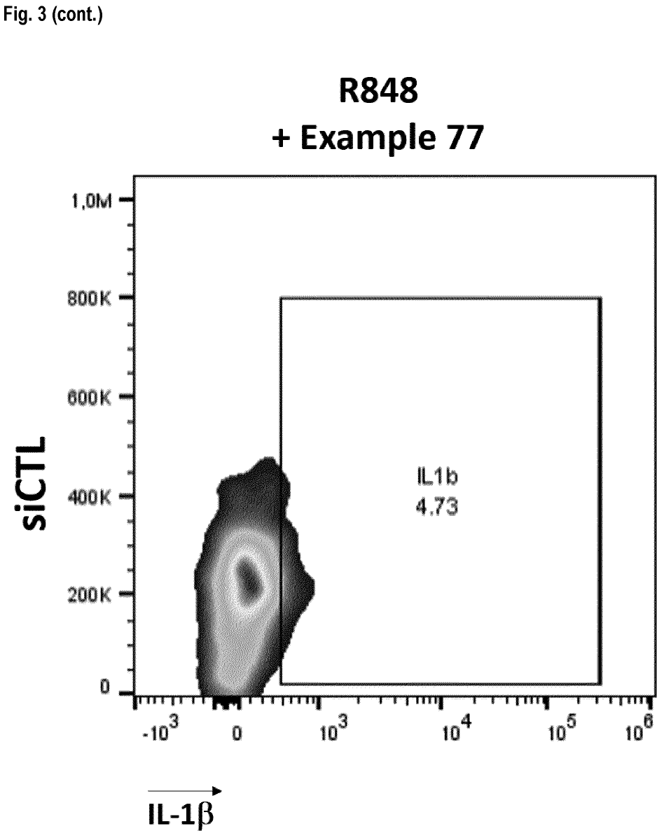

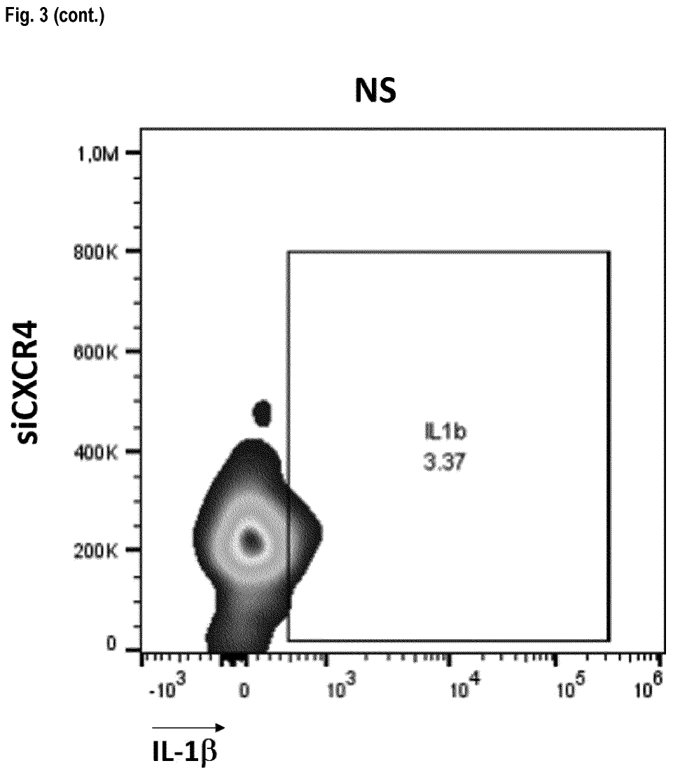

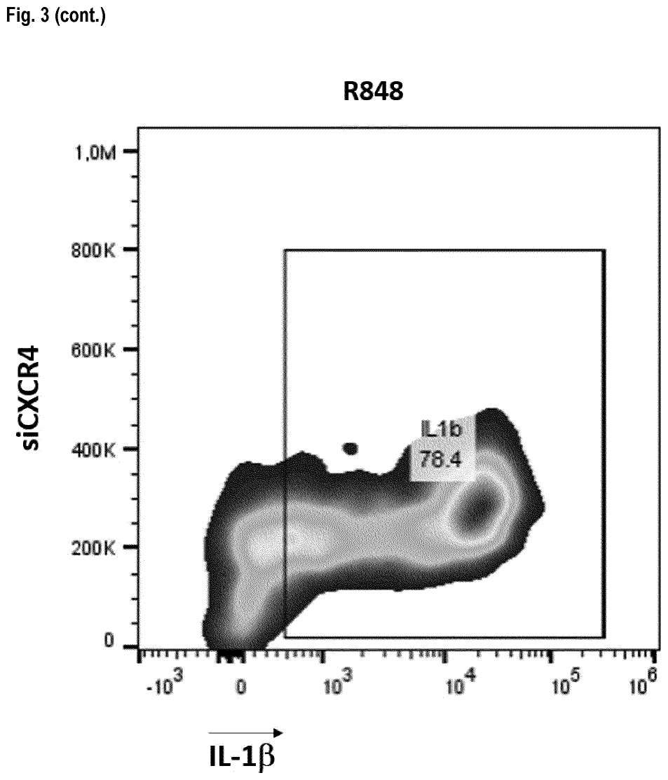

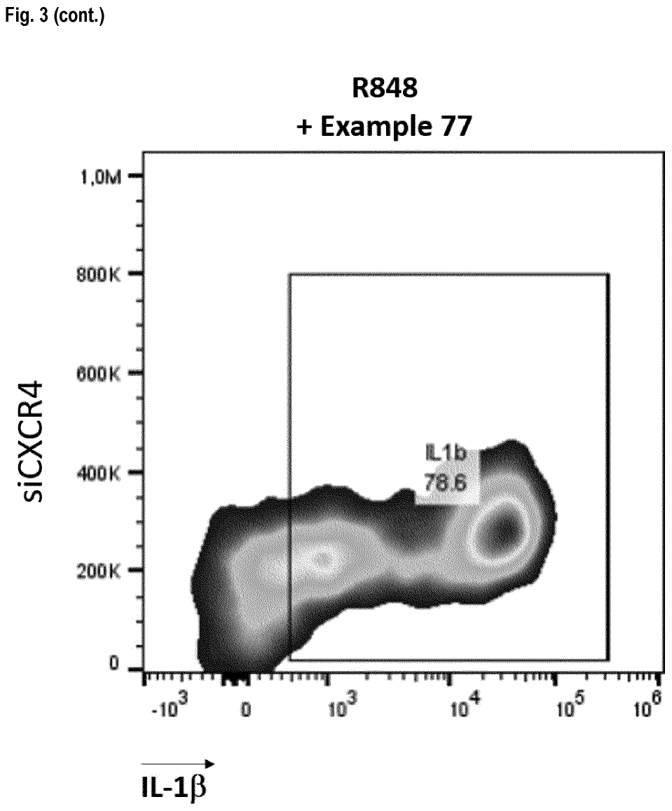

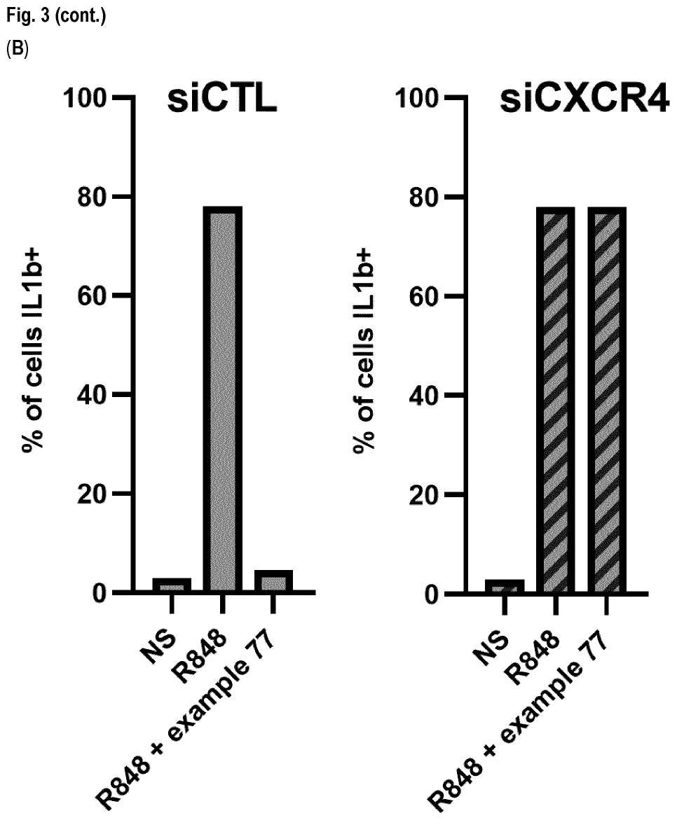

FIG. 3: The anti-inflammatory properties of an exemplary compound of formula (I), i.e. Example 77, are dependent on the chemokine receptor CXCR4. The expression of the CXCR4 receptor was repressed by a specific siRNA (siCXCR4) in healthy donor monocytes. A control siRNA (siCTL) was used as a negative control for the experiment. The monocytes were then treated with Example 77 at 50 nM and activated with R848. Intracellular IL-1p production was measured by flow cytometry. (A) Dot plot representation. (B) Histogram representation of the % of IL-1p positive cells. See Example 202.

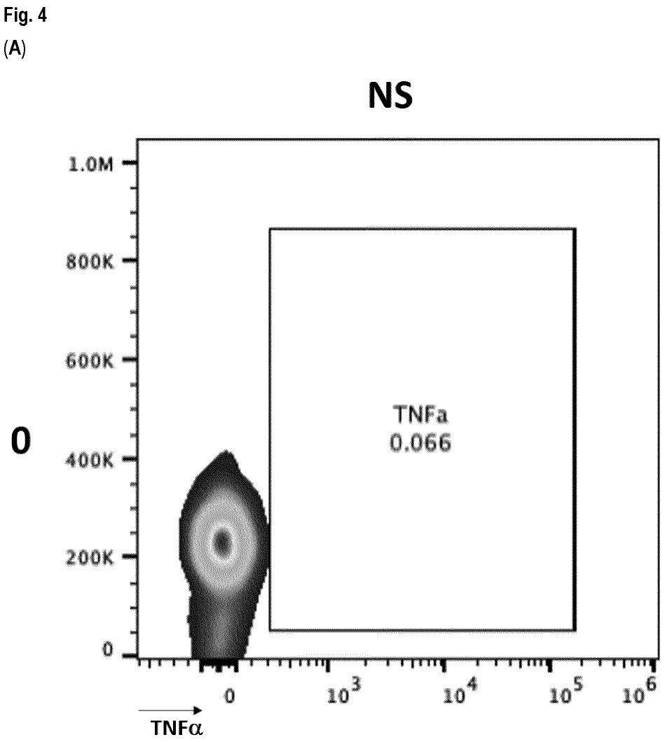

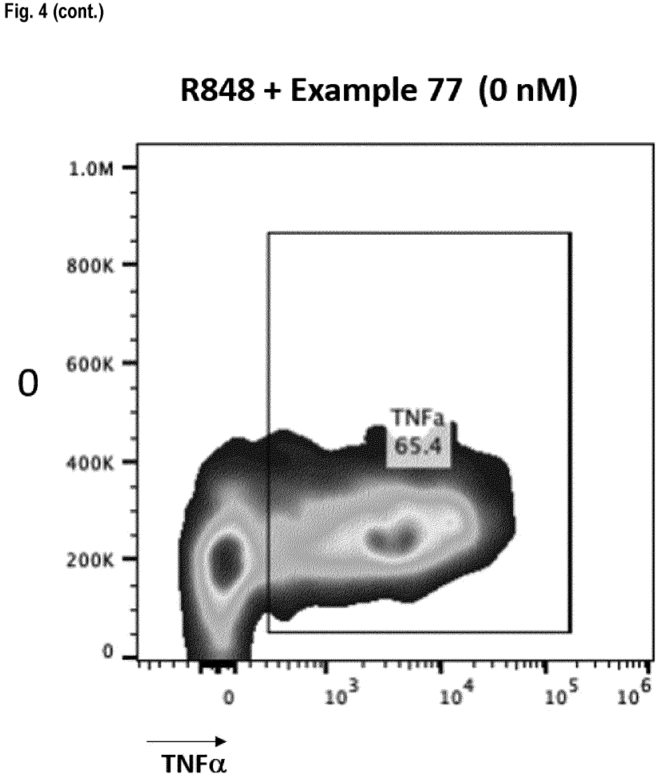

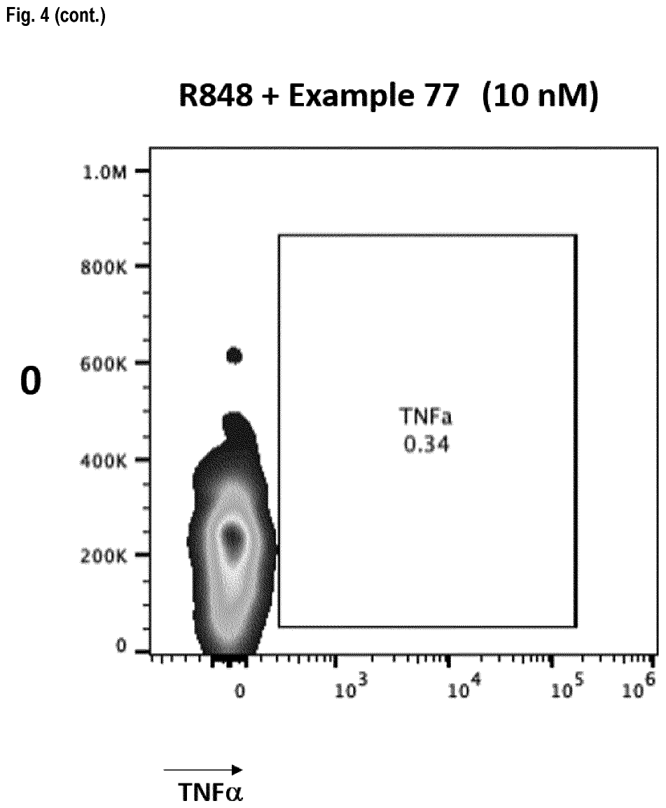

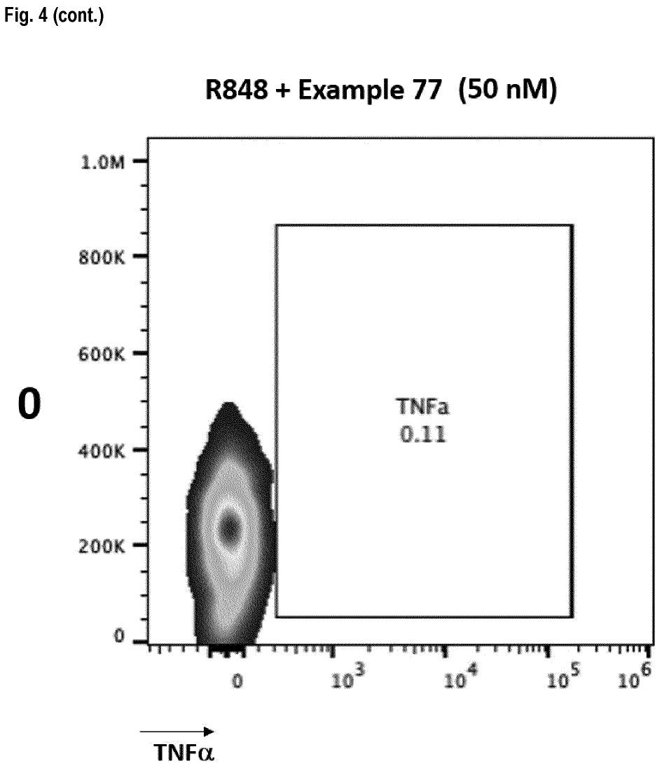

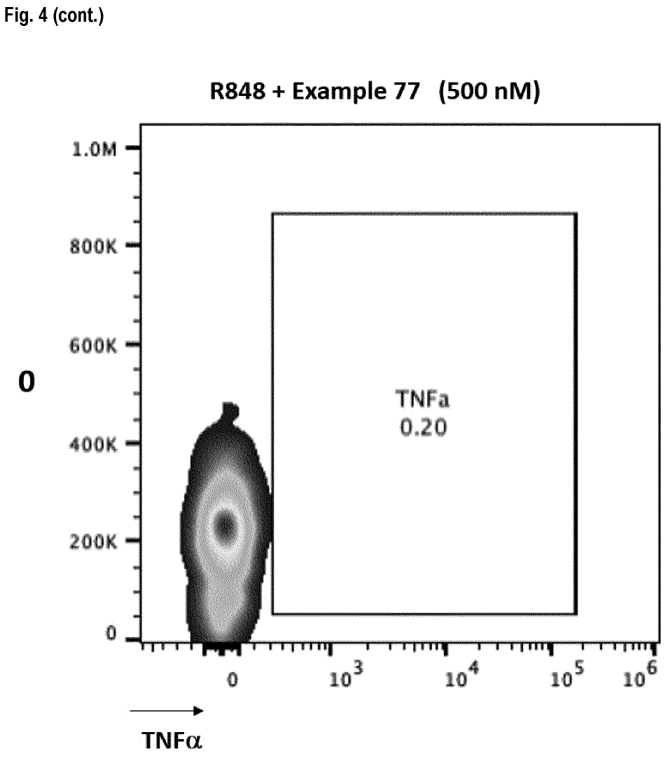

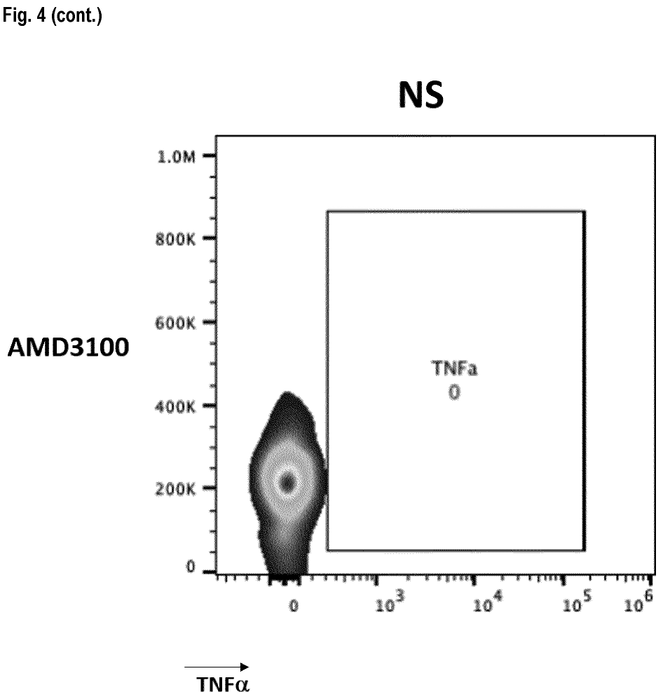

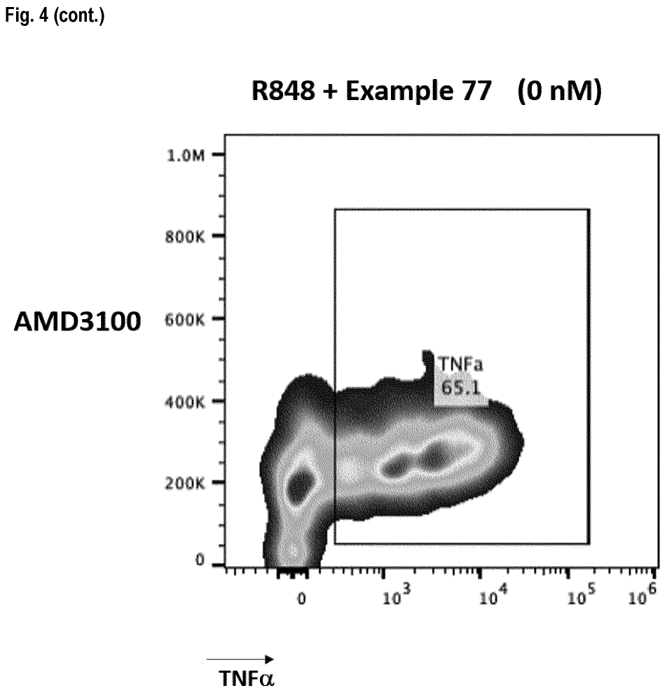

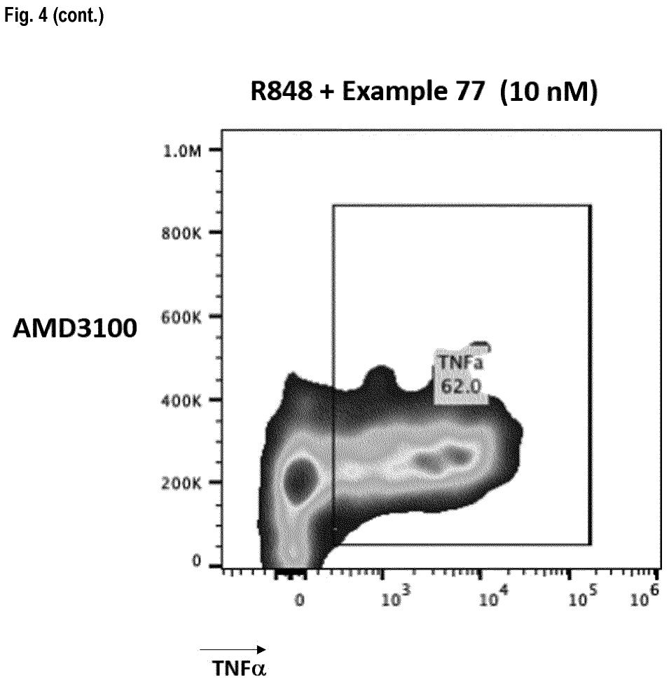

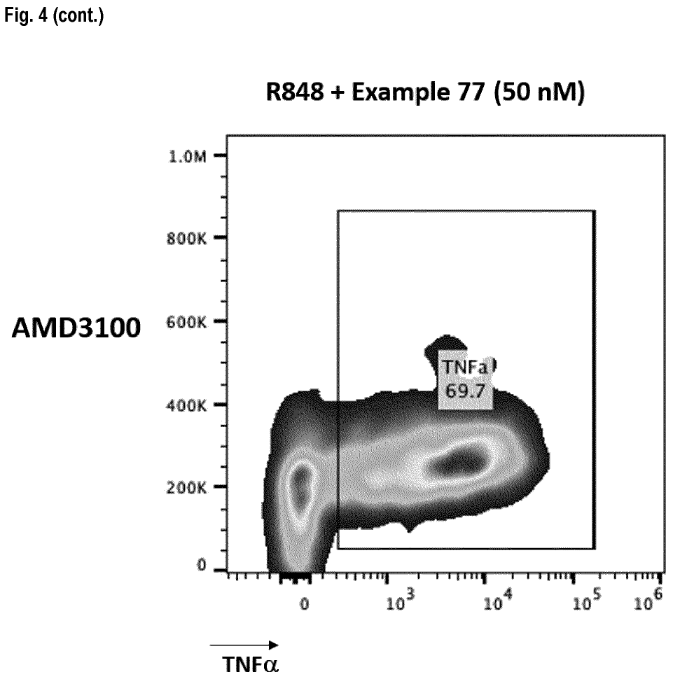

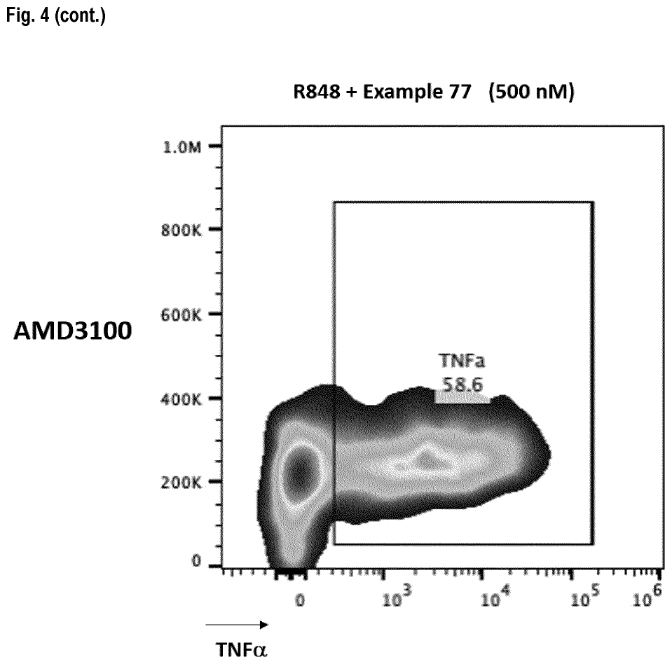

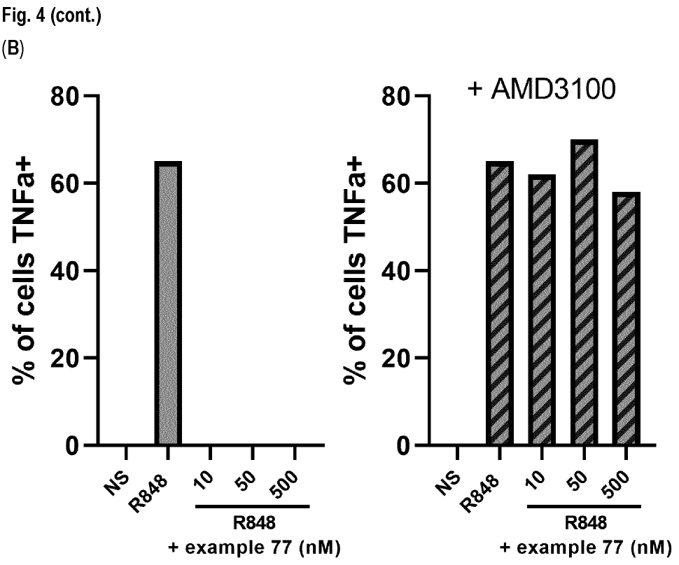

FIG. 4: The anti-inflammatory properties of an exemplary compound of formula (I), i.e. Example 77, are dependent on the chemokine receptor CXCR4. Isolated monocytes from healthy donors were cultured in presence or not of a CXCR4 antagonist, AMD3100, at 20 mM, then treated with increased concentrations (10, 50, 500 nM) of Example 77 and activated with R848. Intracellular level of TNFα was evaluated by flow cytometry. (A) Dot plot representation (B) Histogram representation of the % of TNFα positive cells. See Example 202.

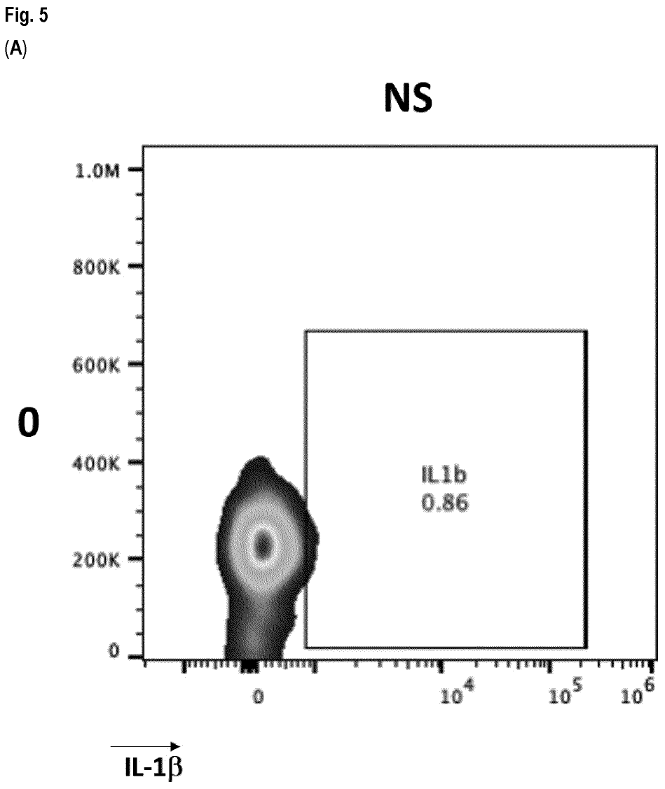

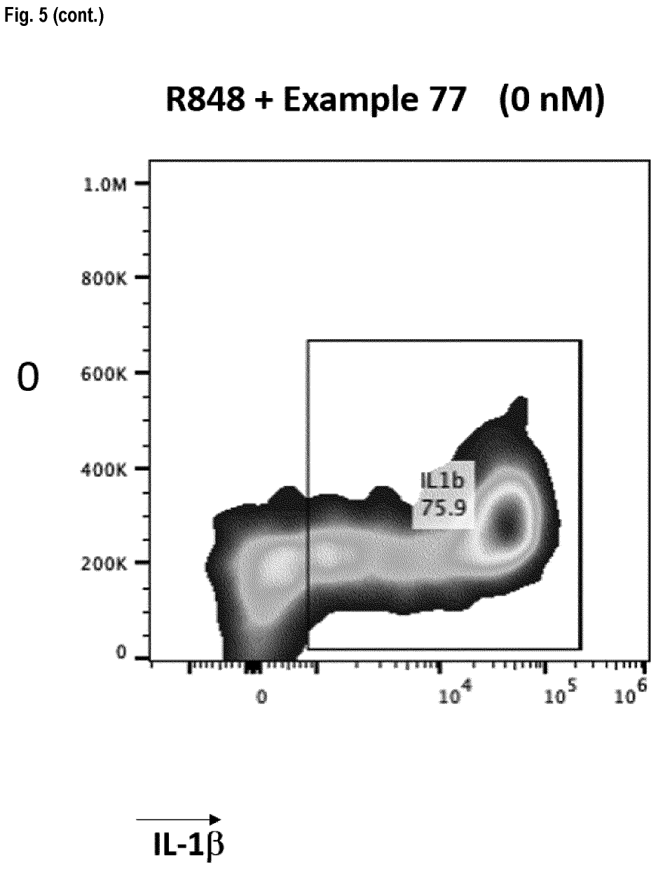

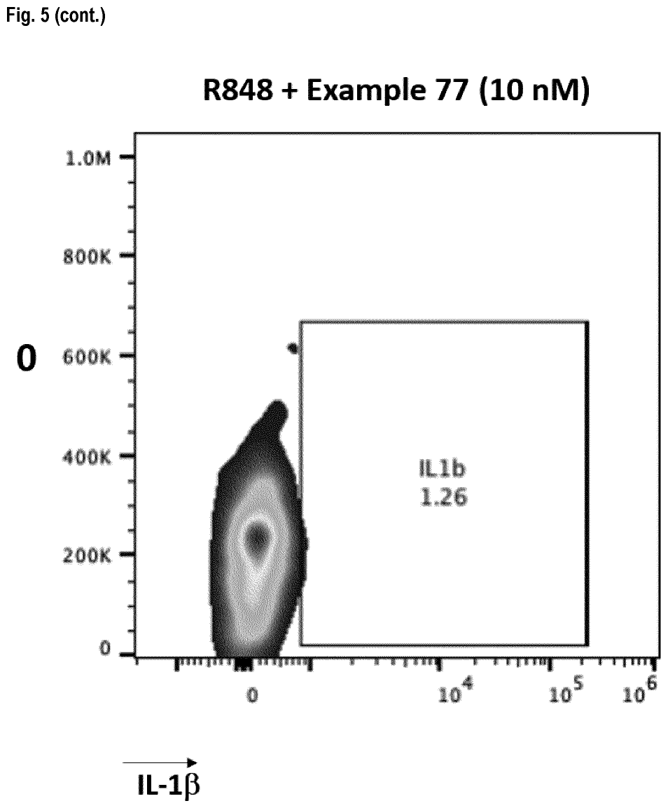

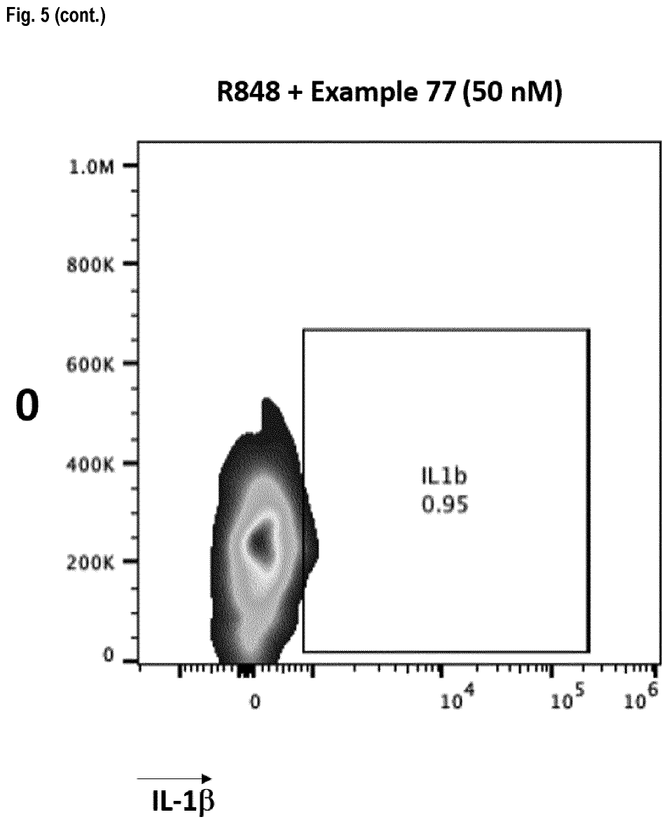

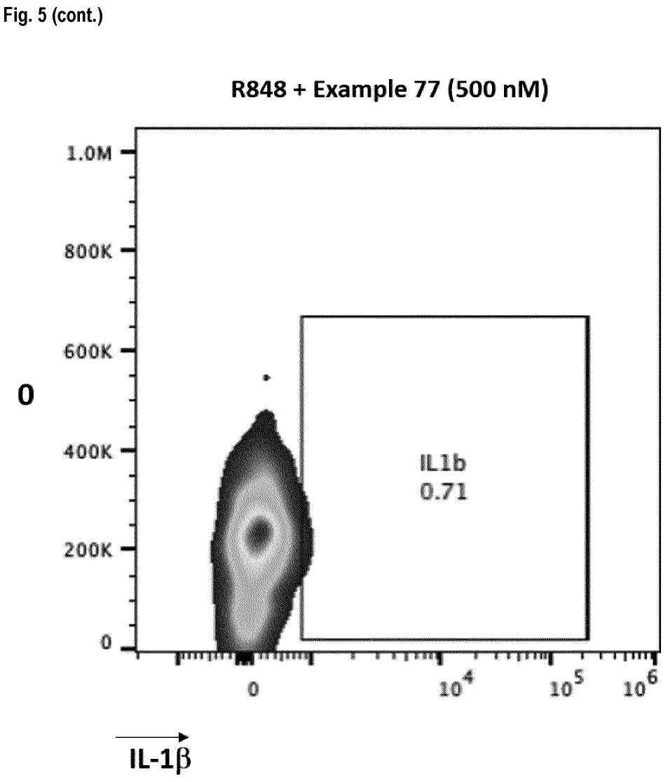

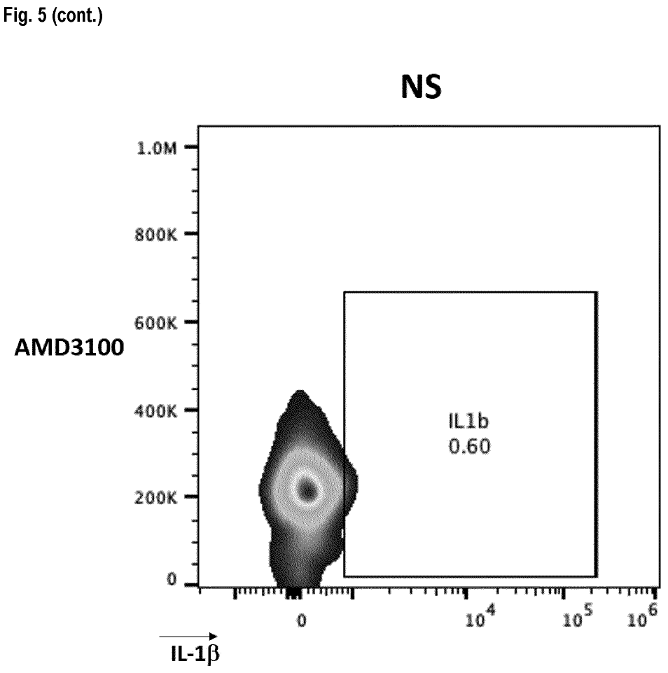

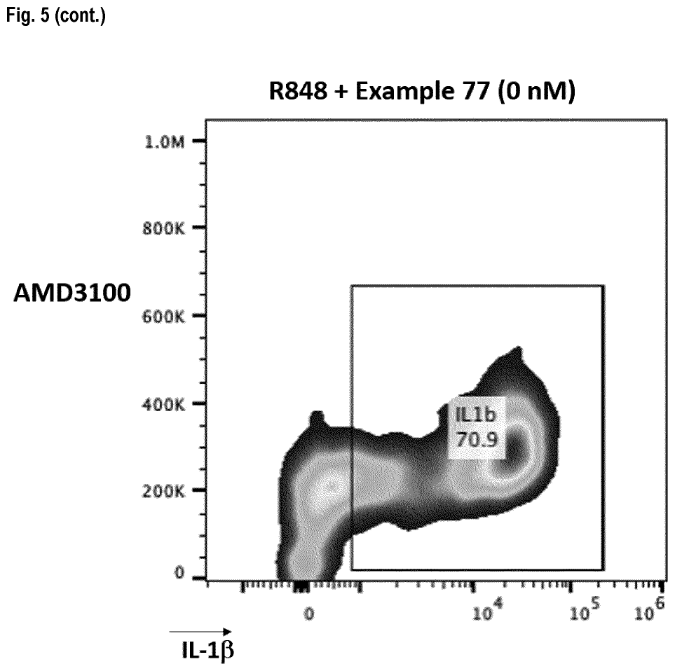

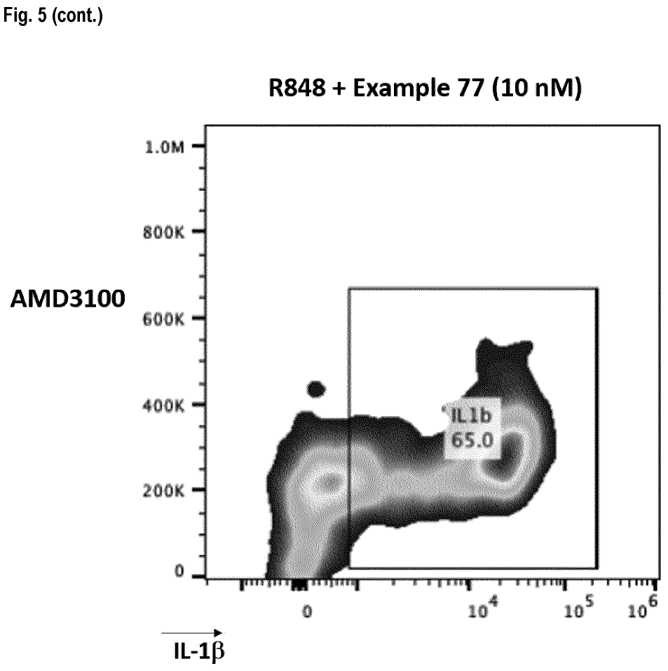

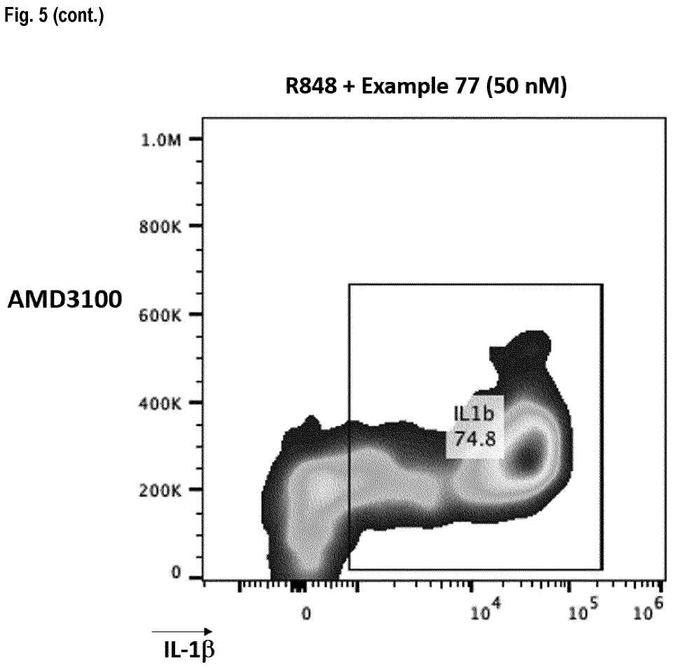

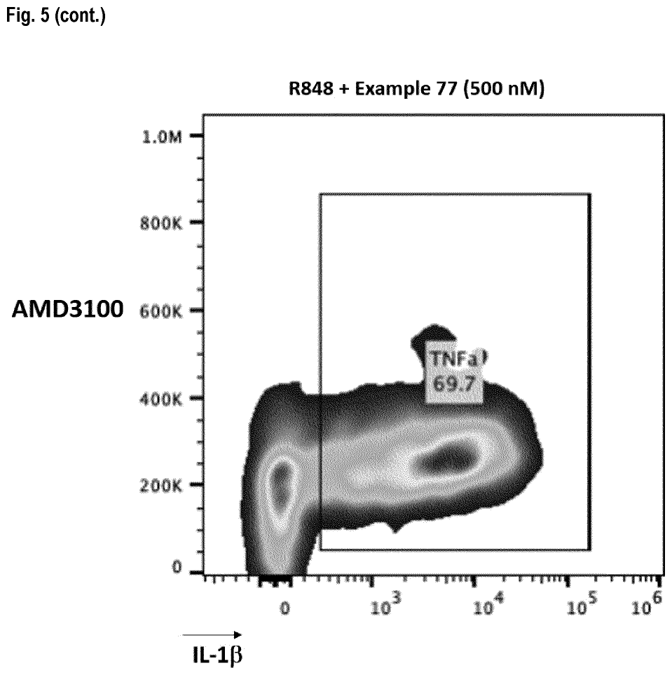

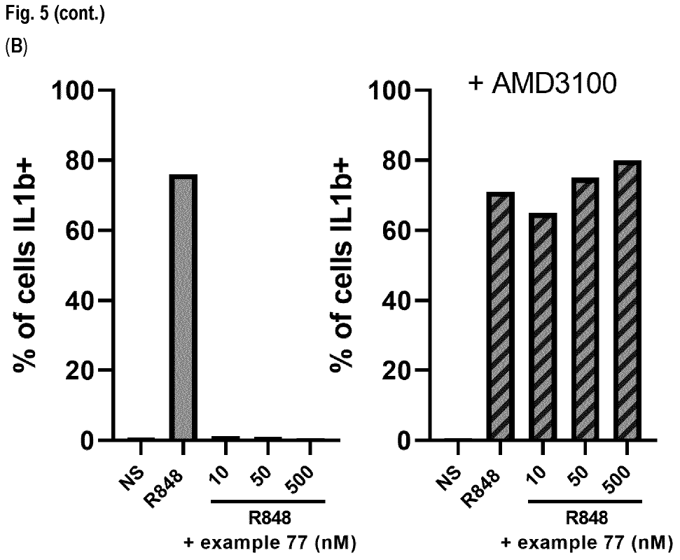

FIG. 5: The anti-inflammatory properties of an exemplary compound of formula (I), i.e. Example 77, are dependent on the chemokine receptor CXCR4. Isolated monocytes from healthy donors were cultured in presence or not of a CXCR4 antagonist, AMD3100, at 20 mM, then treated with increased concentrations (10, 50, 500 nM) of Example 77 and activated with R848. Intracellular level of IL-1p was evaluated by flow cytometry. (A) Dot plot representation (B) Histogram representation of the % of IL-1p positive cells. See Example 202.

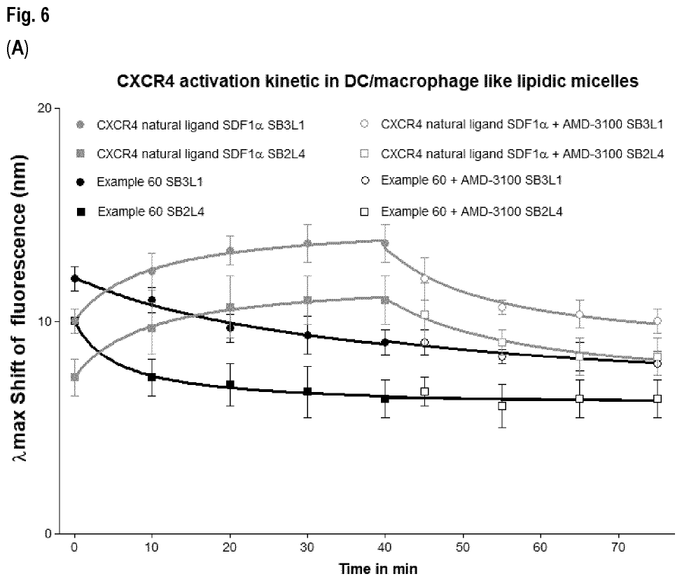

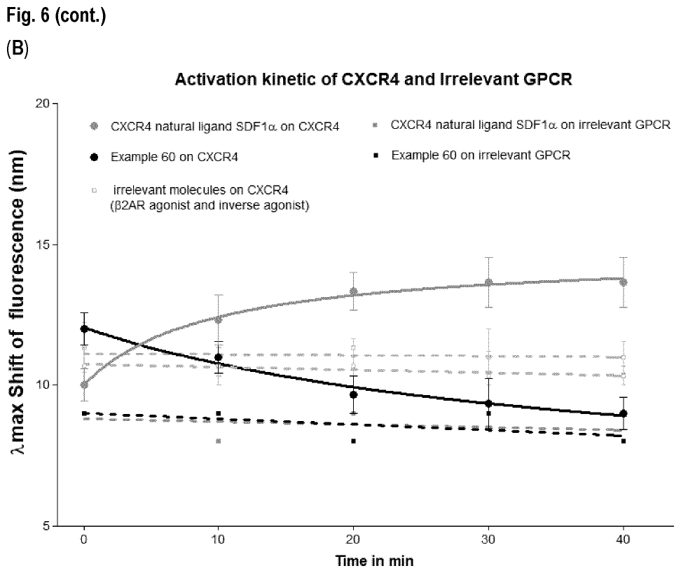

FIG. 6: (A) CXCR4 conformational changes (activation/inactivation spectra as measured by Amax shift in fluorescence (nm)) induced by CXCR4 benchmark molecules SDF1α (15 μM) followed by AMD3100 (100 μM), and an exemplary compound of formula (I), i.e. Example 60 (150 μM), followed by AMD3100 (100 μM) in two dendritic cell/macrophage like lipidic micelles (SB3L1 and SB2L4). (B) Induction of CXCR4 conformational changes (activation/inactivation spectra as measured by Amax shift in fluorescence (nm)) in dendritic cell/macrophage like lipidic micelles (SB3L1) by benchmark molecule SDF1α (15 μM) and an exemplary compound of formula (I), i.e. Example 60 (150 μM), which are not observed when using an irrelevant GPCR. Also, irrelevant molecules (β2AR ligands agonist Norepinephrine (150 μM) and inverse agonist ICI118551 (150 μM) do not induce conformational changes on CXCR4. See Example 202.

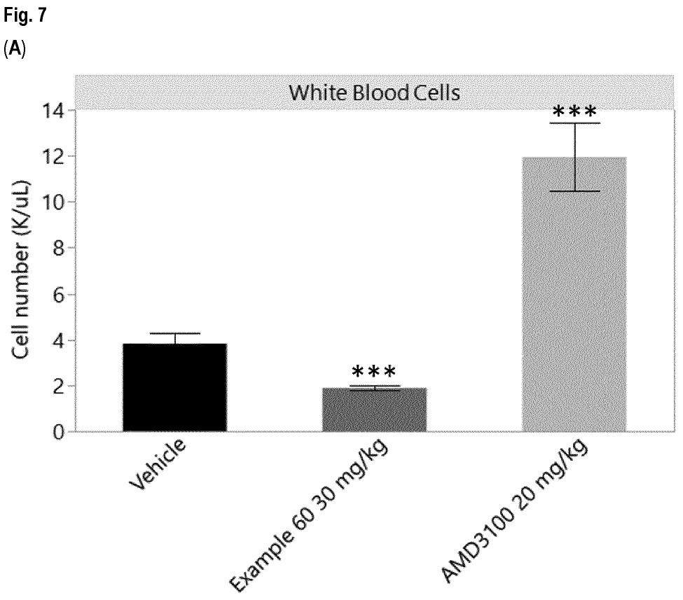

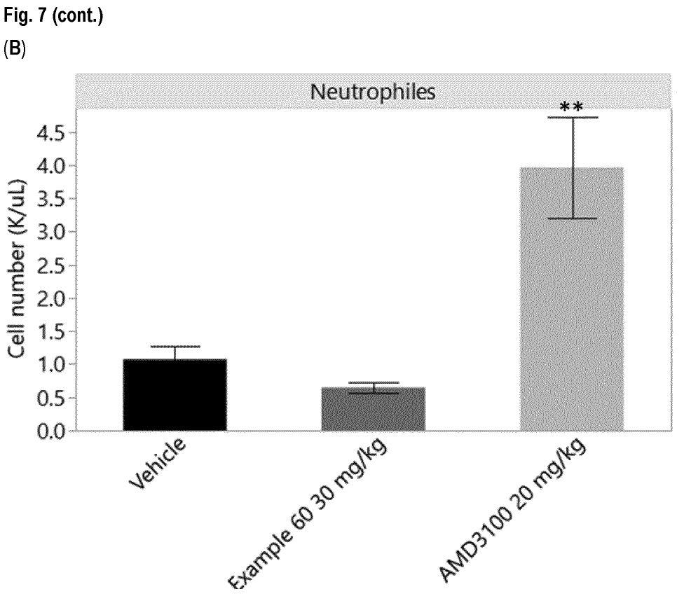

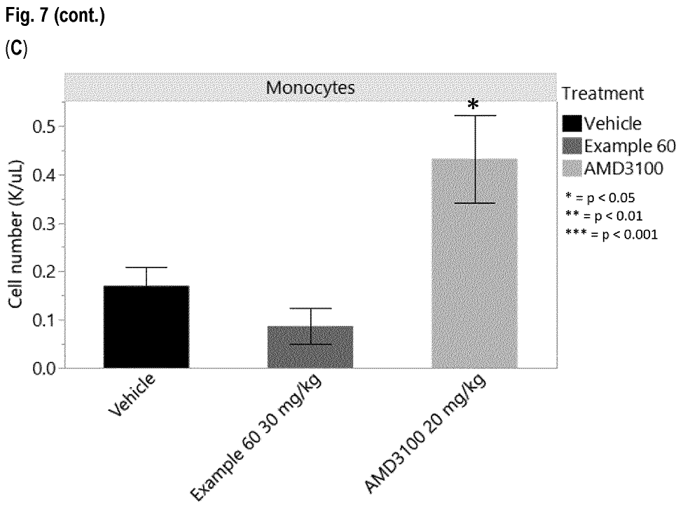

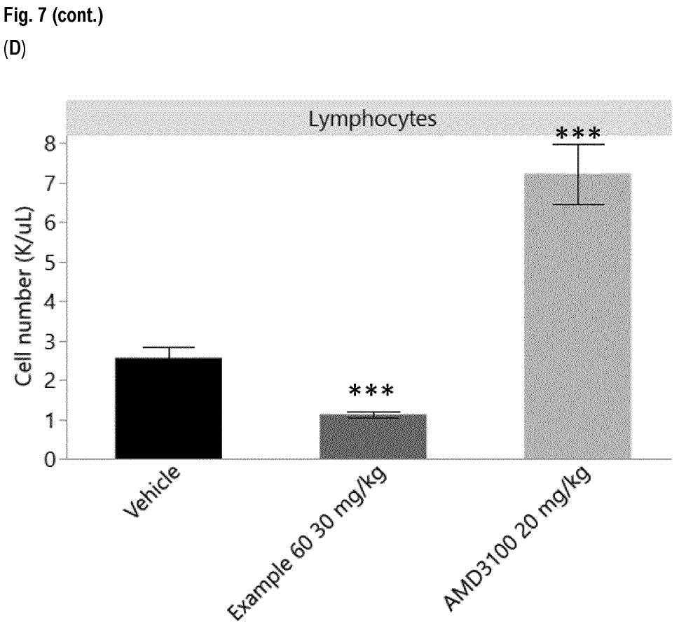

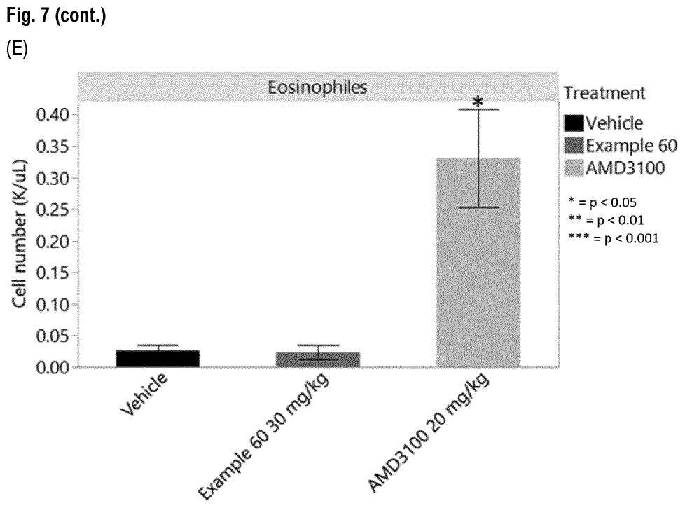

FIG. 7: The absence of CXCR4 antagonistic properties of an exemplary compound of formula (I), i.e. Example 60, in a mouse model. Male C57BL/6 Rj mice show significantly increased numbers of different immune cell types in the blood 2.5 hours after injection with AMD3100 (20 mg/kg, i.p.), a CXCR4 antagonist. This mobilisation of immune cells to the blood is not observed when injecting a vehicle control or Example 60 (30 mg/kg i.p.) as shown for white blood cells (A), neutrophils (B), monocytes (C), lymphocytes (D) and eosinophils (E). Cell numbers are expressed as K/μL. *=p<0.05; **=p<0.01; ***=p<0.001. See Example 202.

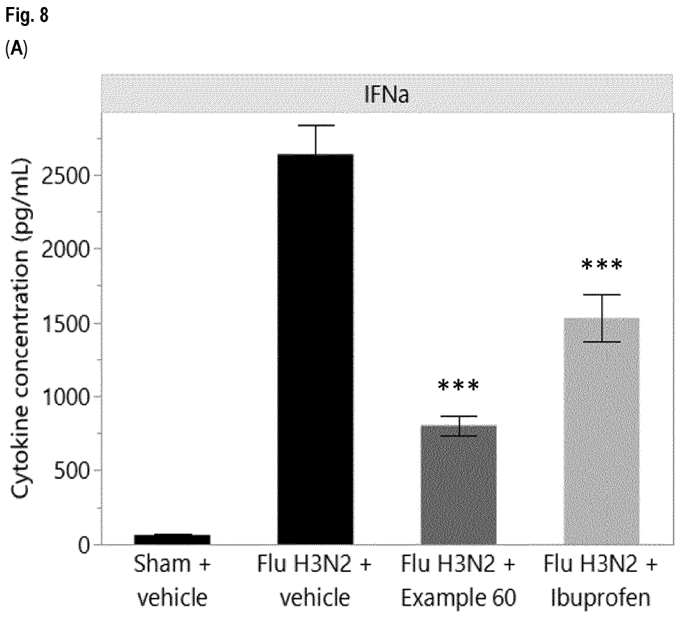

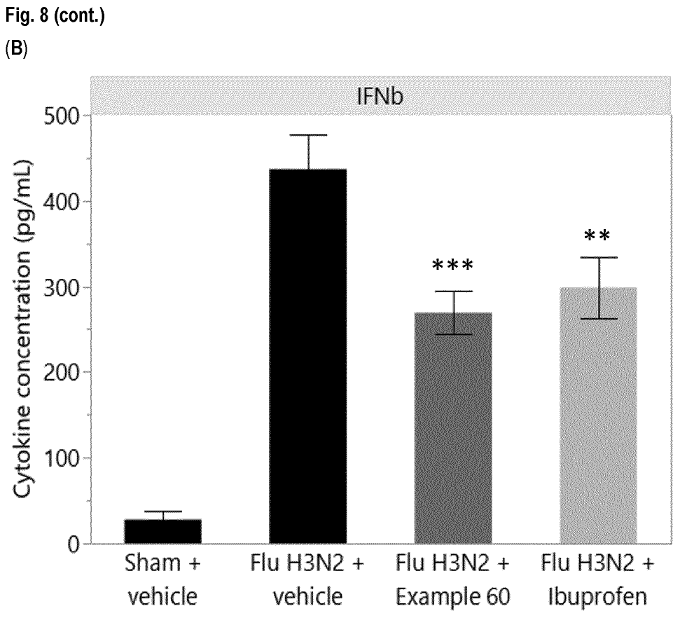

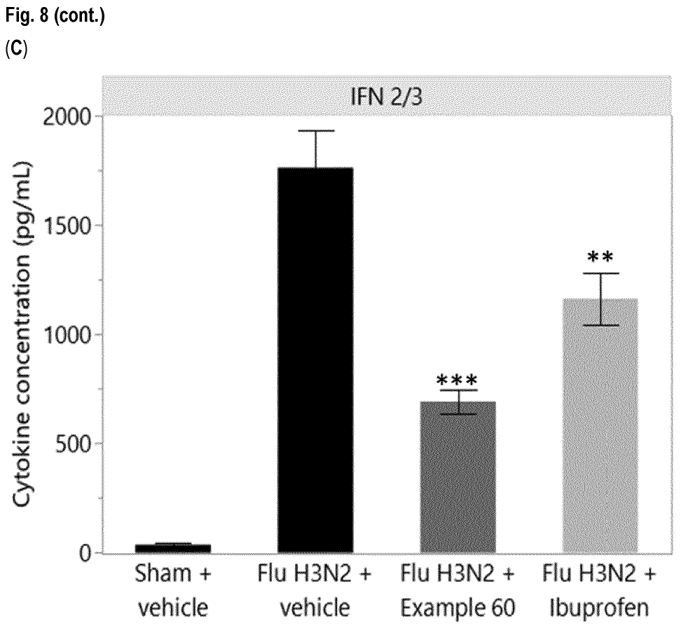

FIG. 8: The anti-inflammatory properties of an exemplary compound of formula (I), i.e. Example 60, in an acute inflammation mouse model. Male 129S8 mice show significantly increased levels of type 1 IFNs in the BALF 3 days after infection with influenza strain H3 N2 (X31) versus sham infection. By a single intranasal administration of ibuprofen (750 μg), a known anti-inflammatory agent, or Example 60 (450 μg) 18 hours before infection with influenza strain H3 N2 (X31), significantly lower concentrations (pg/mL) of IFNα (A), IFNβ (B) and IFNλ2/3 (C) as measured by ELISA are detected in the BALF compared to treatment with vehicle (PBS) in influenza-infected mice. *=p<0.05; **=p<0.01; ***=p<0.001. See Example 202.

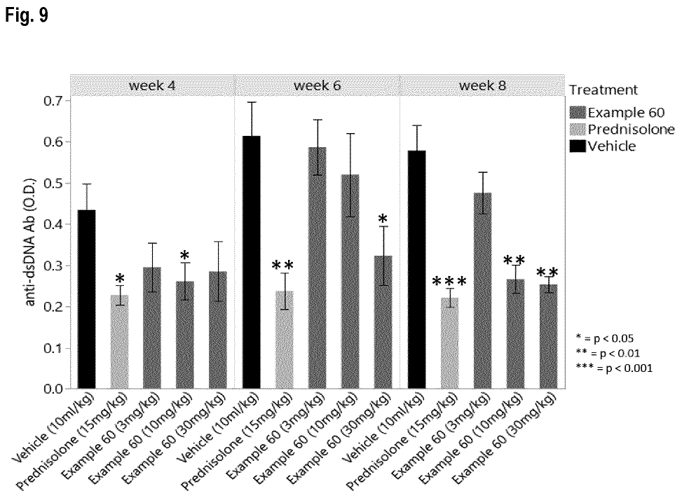

FIG. 9: The effect on anti-dsDNA Ab titers of an exemplary compound of formula (I), i.e. Example 60, in a pristane-induced lupus mouse model. Female Balb/c mice show significantly increased titers of anti-dsDNA Ab after a single injection with pristane and daily vehicle treatment as of Day 1 i.p., as measured as of week 4 in serum. Daily administration as of Day 1 of prednisolone (p.o., 15 mg/kg), a known anti-inflammatory agent, or Example 60 (i.p., at a dose of 3 mg/kg, 10 mg/kg or 30 mg/kg) showed (significantly) decreased anti-dsDNA Ab titers as of week 4 as measured by ELISA compared to the vehicle-treated mice. *=p<0.05; **=p<0.01; ***=p<0.001. See Example 202.

The invention will now be described by reference to the following examples which are merely illustrative and are not to be construed as a limitation of the scope of the present invention.

### EXAMPLES

The compounds/examples described in this section are defined by their chemical formulae and their corresponding chemical names. In case of conflict between any chemical formula and the corresponding chemical name indicated herein, the present invention relates to both the compound/example defined by the chemical formula and the compound/example defined by the chemical name, and particularly relates to the compound/example defined by the chemical formula.

### GENERAL EXPERIMENTAL PROCEDURES

1) General Synthetic Pathway

a) Preparation of Examples of General Formula (I) with n=0

Exemplary compounds of general formula (I) and their pharmaceutically acceptable salts can be synthesized for example, but not only, according to a method adapted from the work of Gebhard Thoma and Emanuel Escher (Thoma G et a1., J Med Chem 2008, 51, 7915; Mona C E et a1., Org Biomol Chem 2016, 14, 10298), as illustrated in the following scheme:

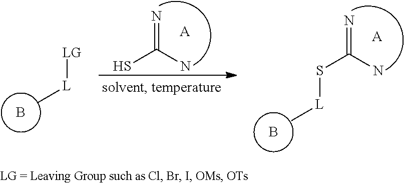

The electrophile (B-L-LG) can react with a cyclic thiourea in an appropriate solvent (such as MeCN, EtOH, DMF or DMA, or mixtures of these) at the suitable temperature (25 to 110° C., preferably 80° C.), optionally in the presence of sodium or potassium iodide until completion of the reaction (preferably overnight) to afford the desired alkylated thiourea.

Alternatively, the electrophile (B-L-LG) can react with a cyclic thiourea in an appropriate solvent (such as THF, MeCN, EtOH, DMF or DMA, or mixtures of these) at the suitable temperature (25 to 80° C., preferably 70° C.), in the presence of a base such as sodium hydride, potassium carbonate, triethylamine, or potassium tert-butoxide until completion of the reaction (preferably overnight) to afford the desired alkylated thiourea. In addtion, when desired, the obtained alkylated product can be further functionalized, for example by deprotection and/or alkylation.

Alternatively, exemplary compounds of general formula (I) and their pharmaceutically acceptable salts can be prepared for example, but not only, as follows:

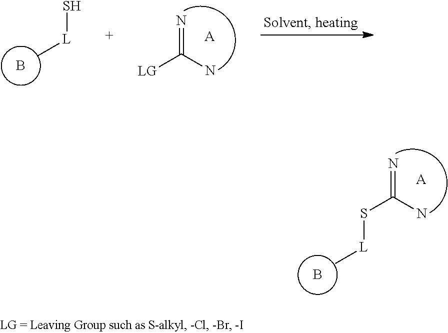

A thiol B-L-SH can react by nucleophilic substitution on an appropriate electrophile such as a S-alkylated thiourea, or a cyclic carbamimidic halogen derivative in an appropriate solvent such as MeCN, DMF or DMA at a suitable temperature (typically 25° C. to 80° C.), optionally in the presence of a base such as sodium hydride, potassium carbonate, triethylamine, or potassium tert-butoxide until completion of the reaction to afford the desired alkylated thiourea. In addtion, when desired, the obtained alkylated product can be further functionalized, for example by deprotection and/or alkylation.

Alternatively, examples with A=A3, A4, A5 and A6 can be prepared for example, but not only, from the corresponding examples with A=A2, under oxidizing conditions using an oxidant such as DDQ, in a suitable solvent such as toluene or MeCN, at a suitable temperature (typically 25° C. to 120° C.). Similarly, examples with A=A8, A9, A10 and A11 can be prepared for example, but not only, from the corresponding examples with A=A7.

b) Preparation of Examples of General Formula (I) with n=1 or 2

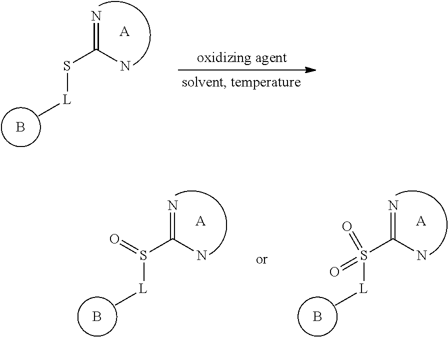

The examples of general formula (I) with n=1 or 2 can be prepared for example, but not only, from the examples of general formula (I) with n=0 by reaction with the right amount (preferably 1 equivalent for n=1 and 2 equivalents for n=2) of an appropriate oxidizing agent in a suitable solvent (e.g. 3-chloroperbenzoic acid in dichloromethane or dihydrogen peroxide in water or methanol).

### c) Preparation of the Starting Electrophiles

For examples with

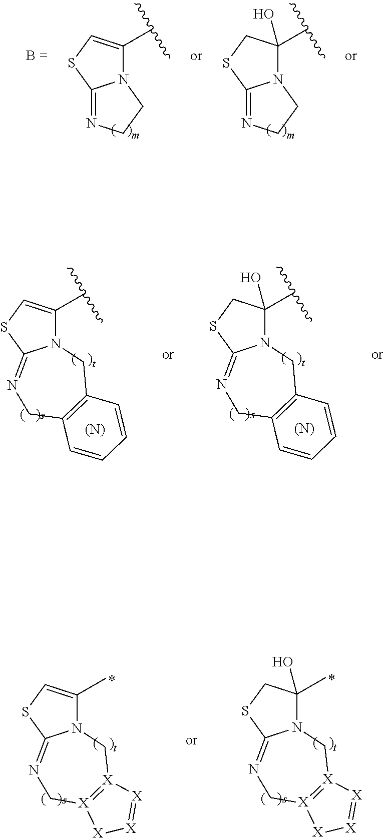

and L=(CH₂)ₘ, and LG=Cl, the electrophile (B-L-LG) can be prepared as follows:

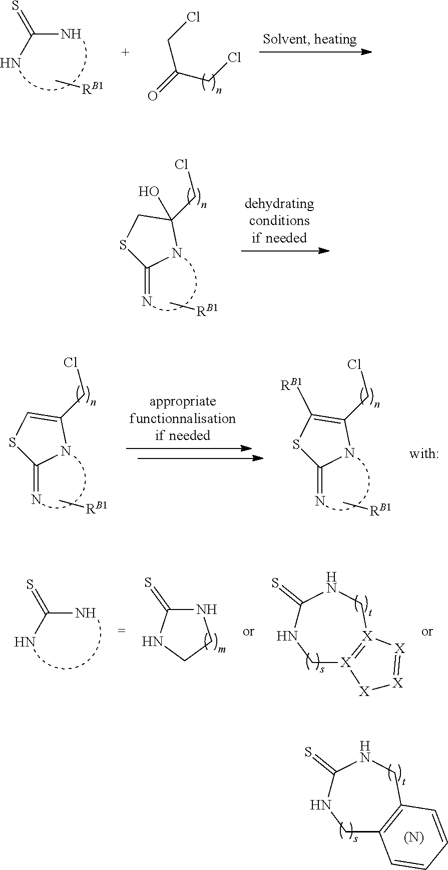

A thiourea can react with a dichloro-ketone in an appropriate solvent such as MeCN, DMF or DMA at a suitable temperature (typically 25° C. to 80° C.). If the dehydration has not occurred yet (typically at low temperature, or when 6 or 7-membered ring cyclic thioureas are used) the hydrated intermediate can be isolated as such, or it can be further reacted under dehydrating conditions such as addition of molecular sieve and/or stronger heating (typically 110° C. in MeCN), or heating in an acidic medium such as HCl in dioxane and/or in DMF and/or in DMA. Finally, when necessary, the dehydrated bicyclic electrophile can be further functionalized, for example by halogenation, optionally followed by further functionalization for example via pallado- or copper-catalyzed coupling such as Suzuki (Maluenda et a1., Molecules 2015, 20, 7528), Stille or Neigishi (Haas et a1., ACS Catal. 2016, 6, 1540) coupling.

d) Preparation of the Starting Cyclic Thioureas

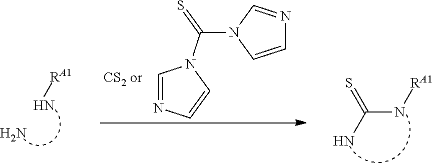

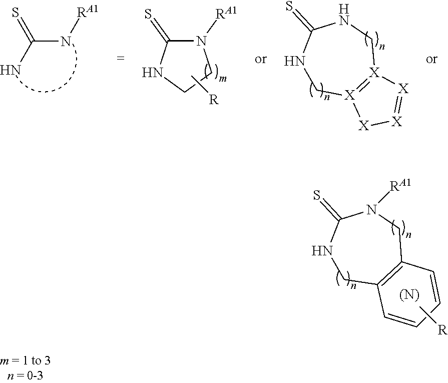

The starting cyclic thioureas can be cyclized from the appropriate diamine or its salt (typically the dihydrochloride) in the presence of di(1H-imidazol-1-yl)methanethione or carbon disulfide and optionally of a base such as triethylamine (preferably when the diamine salt is used) in the appropriate solvent (preferably dichloromethane).

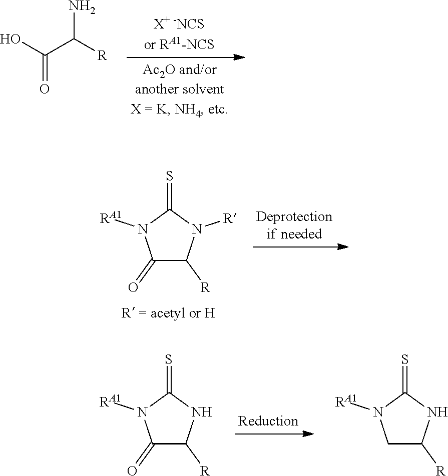

Alternatively, the 5-membered cyclic thioureas can be synthesized from the appropriate amino acid by cyclisation with a thiocyanate (optionally in presence of acetic anhydride followed by a deprotection step in an acidic medium such as HCl in methanol or in water), followed by a reduction step in the presence of a reducing agent such as LAH (O'Donovan et a1, Tetrahedron Letters 2012, 53, 4532).

e) Preparation of the Starting Diamines

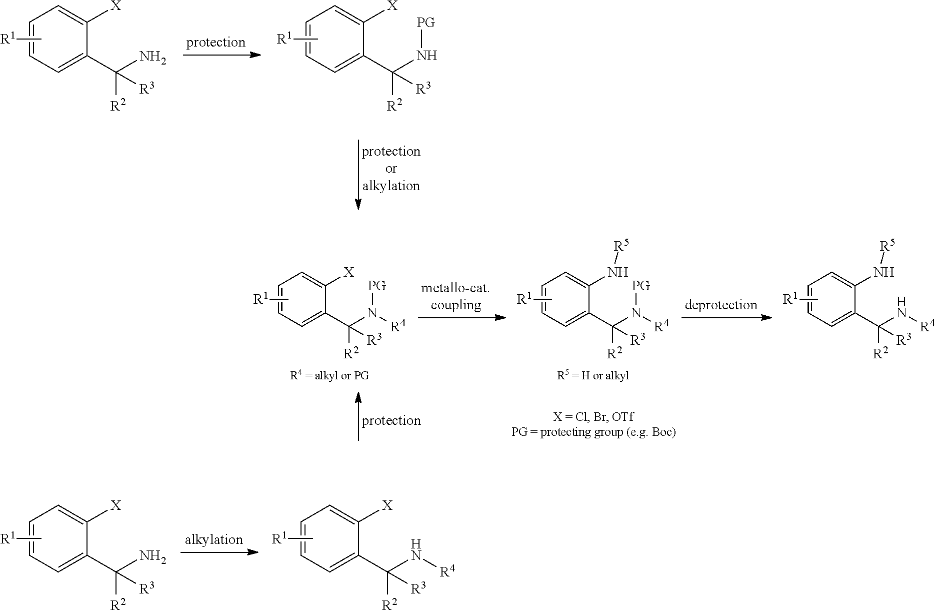

For the formation of 3,4-dihydroquinazoline-2(1H)O-thiones), the corresponding diamines can be synthesized from the appropriate (2-halogeno)benzylamine or its salt or the appropriate 2-(aminomethyl)phenyl triflate, via a protection step, typically using Boc₂O in an appropriate solvent (such as DCM or THE) optionally in the presence of a base (such as triethylamine or DIEA), followed by an alkylation step with an alkylating agent in the presence of a strong base (such as NaH or tBuOK), or by a protection step typically using Boc₂O in presence of stoichiometric DMAP in an appropriate solvent (such as THE). Alternatively, the appropriate (2-halogeno)benzylamine or the appropriate 2-(aminomethyl)phenyl triflate can be first alkylated with an alkylating agent, or via reductive amination, and then protected typically using Boc₂O in an appropriate solvent (such as DCM or THE). Then the second amine car be introduced by a metallo-catalyzed coupling such as a Buchwald-Hartwig amination with an amine or with tert-butyl carbamate, typically using a catalyst (such as XPhos Pd G4, XPhos Pd G2, BrettPhos Pd G4, Xantphos Pd G4 or BINAP+Pd₂dba₃) in the presence a base (such as Cs₂CO₃ or NaOtBu) in an appropriate solvent (such as dioxane or toluene) at the suitable temperature (such as 80 to 110° C.) (Surry, D. S. & Buchwald, S. L. Chem. Sci. 2011, 2, 27). Then a deprotection step can lead to the desired diamine. If PG=Boc, acidic deprotection conditions can be used, for example HCl in dioxane, or TFA in DCM.

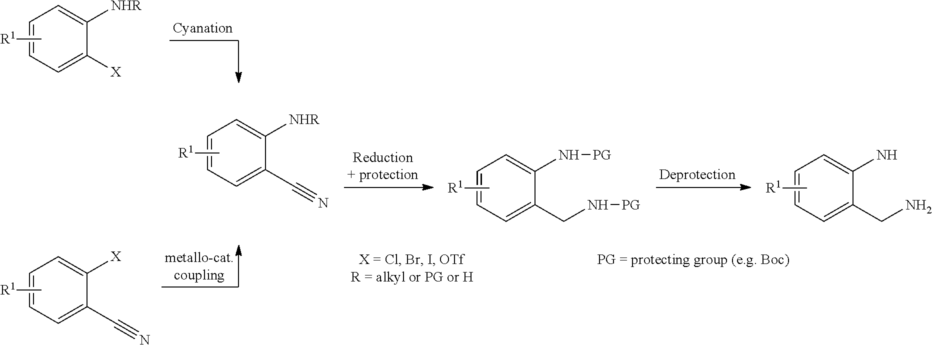

Alternatively the 3,4-dihydroquinazoline-2(1H)-thiones can be synthesized from the appropriate 2-halogeno-aniline or 2-aminophenyl triflate by a cyanation reaction, typically using Zn(CN)₂ as a cyanide source and bis(tri-tert-butylphosphine)palladium(0) as a catalyst in an appropriate solvent (such as dioxane, DMF or DMA) at the suitable temperature (such as 80° C.-110° C.). Alternatively the 3,4-dihydroquinazoline-2(1H)-thiones can be synthesized from the appropriate 2-halogenobenzonitrile or 2-cyanophenyl triflate by a metallo-catalyzed coupling such as a Buchwald-Hartwig amination with an amine or with tert-butyl carbamate, typically using a catalyst (such as XPhos Pd G4, BrettPhos Pd G4, Xantphos Pd G4 or BINAP+Pd₂dbas) in the presence a base (such as Cs₂CO₃ or NaO^{t}Bu) in an appropriate solvent (such as dioxane or toluene) at the suitable temperature (such as 80 to 110° C.) (Surry, D. S. & Buchwald, S. L. Chem. Sci. 2011, 2, 27). Then a one-pot reduction+protection step can afford the corresponding protected diamine in the presence of a reducing and a protecting agent (such as NaBH₄ and CoCl₂ or NiCl₂ in presence of Boc₂O) in an appropriate solvent (such as MeOH), followed by a deprotection step to lead to the desired diamine. If PG=Boc, acidic deprotection conditions can be used, for example HCl in dioxane, or TFA in DCM.

2) General Conditions

All reagents were commercial grade and used without further purification. Reactions were typically run using commercial anhydrous solvents under argon atmosphere.

Column chromatography was generally performed with a Biotage Isolera Four or a Biotage Isolera One apparatus using Interchim PURIFLASH jumbo pack silica HP cartridges pre-filed with 50 μm silica gel, or A.I.T. France empty columns packed with Merck Geduran® Si 60 (40-63 μm) silica gel. When specified otherwise, Interchim® PURIFLASH jumbo pack silica HP cartridges pre-filed with 15 μm silica gel or Interchim® PURIFLASH jumbo pack silica SDT cartridges pre-filed with 20 μm silica gel or Biotage Sfär KP-Amino D cartridges pre-filed with 50 μm silica gel could be used when necessary.

Releasing of free bases from the corresponding salts was carried out using Biotage ISOLUTE® SCX-2 cation exchange cartridges.

¹H-NMR spectra were recorded on a Bruker AMX-400 or on a Bruker Avance 300 spectrometer. Proton chemical shifts are listed relative to residual CD₃OD (3.31 ppm), DMSO-_{d6} (2.50 ppm) or D₂O (4.78 ppm). Splitting patterns are designated as s (singlet), d (doublet), dd (doublet-doublet), t (triplet), tt (triplet-triplet), td (triplet-doublet), q (quartet), quint (quintuplet), sex (sextuplet), sept (septuplet), m (multiplet), b (broad).

UPLC-MS analyses were recorded with an UPLC Waters Aquity platform with a photodiode array detector (190-400 nm) using an Acquity CSH C₁₈ 1.7 μm (2.1×30 mm) column. The mobile phase consisted in a gradient of water with 0.025% of TFA and acetonitrile with 0.025% of TFA. The flow rate was 0.8 mL per min. All analyses were performed at 55° C. The UPLC system was coupled with a Waters SQD2 platform. All mass spectra were full-scan experiments (mass range 100-800 amu) and were obtained using electrospray ionization.

HPLC-MS were recorded using a HPLC Waters platform with a 2767 sample manager, a 2525 pump, a photodiode array detector (190-400 nm). This HPLC system was coupled with a Waters Acquity QDa detector. All mass spectra were full-scan experiments (mass range 110-850 amu) and were obtained using electro spray ionization. For analytical samples, the selected column was a PF5C18 AQ 5 μm (4.6×250 mm). For preparative purifications, the selected column was either column A an XSelect CSH prep C₁₈ 5 μm (19×100 mm) or column B a PF5C18 AQ 5 μm (21.2×250 mm). The mobile phase in all cases consisted in an appropriate gradient of water with 0.1% of formic acid and acetonitrile with 0.1% of formic acid. The flow rate was 1 mL/min in analytical mode, and in preparative mode 25 mL/min for column A and 21 mL/min for column B. All HPLC-MS were performed at room temperature.

Alternatively, analytical HPLC-MS were recorded using a HPLC Ultimate 3000 platform (Thermo Scientific) with a photodiode array detector (190-800 nm). This HPLC system was coupled with a Bruker HCT, ion trap detector. All mass spectra were full-scan experiments (mass range 110-1100 amu) and were obtained using electro spray ionization (ESI). The selected columns were a PF5C18 AQ 5 μm (4.6×250 mm, flow rate 1 mL/min), a Princeton Spher-60 C8 10 μm (4.6×150 mm, flow rate 1.5 mL/min) and a Syncronis aQ 5 μm (4.6×150 mm, flow rate 1.3 mL/min). All HPLC-MS were performed at room temperature.

Alternatively, preparative HPLC purifications were performed on a PLC 2020 (Gilson) with a photodiode array detector (190-800 nm). The selected columns were column B a PF5C18 AQ 5 μm (21.2×250 mm, flow rate 30 mL/min)), or column C a Princeton Spher-60 C8 10 μm (30×150 mm, flow rate 30 mL/min) or column D a Syncronis AQ 5 μm (20×150 mm, flow rate 20 mL/min). The mobile phase in all cases consisted in an appropriate gradient of water with 0.1% of formic acid and acetonitrile with 0.1% of formic acid. All HPLC were performed at room temperature.

Melting points were measured on a Barnstead Electrothermal 9100 and are not corrected.

Unless mentioned otherwise all compounds isolated by filtration or centrifugation from organic solvents were dried overnight in high vacuum at 50-70° C., and all compounds isolated by filtration from an aqueous medium were dried overnight in high vacuum over P₂O₅.

Pd116 refers to bis(tri-tert-butylphosphine)palladium(0).

Hydrochloride salts of the examples of the invention have been assumed to be mono-, di- or tri-hydrochloride as indicated hereinafter, according to NMR analysis and/or reaction conditions. However, no chlorine titration was performed, therefore the number of HCl associated with these examples may not be fully accurate. The present invention relates to each of the corresponding examples (i.e., each of the corresponding exemplary compounds of formula I) in the form of a hydrochloride salt (including, but not being limited to, the specific HCl salt disclosed or depicted hereinafter), and likewise relates to each of the corresponding examples in non-salt form or in the form of any other pharmaceutically acceptable salt or solvate thereof.

3) General Procedures and Methods:

General Procedure 1a: Dihydrothiazole or Thiazolidinol Formation

A solution of a thiourea (1.0 equiv) and di-chloroketone (1.0-1.5 equiv) in MeCN (0.2 M) was heated for 5 h to overnight at 80° C. The resulting mixture was allowed to cool down to rt, then Et₂O was optionally added to help the precipitation. The resulting precipitate was filtrated and triturated in MeCN. Optionally the filtrate was concentrated to dryness, triturated in cold MeCN and filtrated to recover more product. The product was further purified when necessary.

General Procedure 1b: Dihydrothiazole Formation

A solution of a thiourea (1.0 equiv) and di-chloroketone (1.0-1.5 equiv) in MeCN (0.2 M) was heated overnight at 80° C. The hydrate intermediate was filtrated, washed with MeCN and then suspended in HCl 4 N in dioxane (0.8 M) and stirred at 80° C. for 1 h to 3 days. The resulting suspension was allowed to cool down to rt, then Et₂O was optionally added to help the precipitation. The precipitate was filtrated and triturated in Et₂O. Optionally the filtrate was concentrated to dryness, triturated in cold MeCN and filtrated to recover more product. The product was further purified when necessary.

General Procedure 1c: Dihydrothiazole Formation

A solution of a thiourea (1.0 equiv) and di-chloroketone (1.0-1.5 equiv) in MeCN (0.2 M) was heated overnight at 80° C. The resulting mixture was concentrated to dryness. The product was further purified when necessary.

General Procedure 1d: Dihydrothiazole Formation

A solution of a thiourea (1.0 equiv) and di-chloroketone (1.0-1.5 equiv) in MeCN (0.2 M) was heated overnight at 80° C. The hydrate intermediate was filtrated, washed with MeCN and then suspended in HCl 4 N in dioxane (0.8 M) and stirred at 80° C. for 1 h to 3 days. The reaction mixture was then concentrated to dryness. The product was further purified when necessary.

General Procedure 1e: Dihydrothiazole Formation

A solution of a thiourea (1.0 equiv) and di-chloroketone (1.0-1.5 equiv) in MeCN (0.2 M) was heated overnight at 80° C. The hydrate intermediate was filtrated, washed with MeCN and then suspended in HCl 4 N in dioxane (0.8 M) and stirred at 110° C. for 1 h to 3 days. The reaction mixture was then concentrated to dryness. The product was further purified when necessary.

General Procedure 1f: Dihydrothiazole Formation

A solution of a thiourea (1.0 equiv) and di-chloroketone (1.0-1.5 equiv) in MeCN (0.2 M) was heated overnight at 80° C. The hydrate intermediate was filtrated, washed with MeCN and then suspended in HCl 4 N in dioxane (0.8 M) and stirred at 110° C. for 1 h to 3 days. The resulting suspension was allowed to cool down to rt, then Et₂O was optionally added to help the precipitation. The resulting precipitate was filtrated and triturated in dioxane. Optionally the filtrate was concentrated to dryness, triturated in cold dioxane and filtrated to recover more product. The product was further purified when necessary.

General Procedure 1g: Thiazolidinol Formation

A solution of a thiourea (1.0 equiv) and di-chloroketone (1.0-1.1 equiv) in MeCN (0.2 M) was heated for 3 h to 48 h at 50° C. The resulting precipitate was filtrated, washed with MeCN and optionally triturated in diethyl ether.

General Procedure 1hf: Dihydrothiazole Formation

A solution of a thiourea (1.0 equiv) and di-chloroketone (1.0-1.5 equiv) in MeCN (0.2 M) was heated for 5-20 h at 80° C. The resulting mixture was concentrated to dryness and the residue was dissolved in MeOH (1 mL), precipitated with Et₂O, and the solid was isolated by centrifugation and washed with Et₂O (2×2 mL). The product was further purified when necessary.

General Procedure 2a: Thiourea Formation from Diamine

At 0° C., to a solution of a diamine (1.0 equiv) in DCM (0.6 M) was added a solution of di(1H-imidazol-1-yl)methanethione (1.0 equiv) in DCM (0.4 M). The reaction mixture was stirred at 0° C. to 25° C. for 1-5 h, hydrolyzed with an aqueous saturated solution of NaHCO₃ and extracted several times with DCM. The combined organic extracts were washed with brine, filtered through a hydrophobic cartridge and concentrated to dryness. When necessary the crude was further purified.

General Procedure 2b: Thiourea Formation from Diamine

At 0° C., to a solution of a diamine (1.0 equiv) in DCM (0.6 M) was added a solution of di(1H-imidazol-1-yl)methanethione (1.0-1.1 equiv) in DCM (0.3 M). The reaction mixture was stirred at 0° C. to 25° C. for 1-5 h. Then the suspension was filtrated and the solid was washed with cold DCM. Optionally the filtrate was concentrated to dryness, triturated in cold DCM and filtrated to recover more product. When necessary the crude was further purified.

General Procedure 2c: Thiourea Formation from Diamine

At 0° C., to a suspension of a diamine hydrochloride (1.0 equiv) in DCM (0.2 M) was added triethylamine (2.2 equiv) and the mixture was stirred at 0° C. for 15 min. Then, at 0° C., di(1H-imidazol-1-yl)methanethione (1.0 equiv) was added in one portion. The reaction was stirred at 0° C. to 25° C. for 1-5 h. Then the suspension was filtrated and the solid was washed with cold DCM. When necessary the crude was further purified.

General Procedure 2d: Thiourea Formation from Diamine

At 0° C., to a suspension of a diamine hydrochloride (1.0 equiv) in DCM (0.2 M) was added triethylamine (2.2 equiv) and the mixture was stirred at 0° C. for 15 min. Then, at 0° C., di(1H-imidazol-1-yl)methanethione (1.0 equiv) was added in one portion. The reaction was stirred at 0° C. to 25° C. for 1-5 h and concentrated to dryness. The crude was further purified as detailed hereinafter.

General Procedure 2e: Thiourea Formation from Diamine

At 0° C., to a solution of a diamine (1.0 equiv) in DCM (0.6 M) was added a solution of di(1H-imidazol-1-yl)methanethione (1.0-1.1 equiv) in DCM (0.3 M). The reaction mixture was stirred at 0° C. to 25° C. for 1-16 h and concentrated to dryness. The crude was further purified as detailed hereinafter.

General Procedure 3a: Imidazoline Formation from Amino Acid—Step 1—Cyclisation

A suspension of an amino acid (1.0 equiv) and potassium thiocyanate (1.0 equiv) in acetic acid (1.0 M) and acetic anhydride (1.0 M) was heated to 80° C. for 1-1.5 h. Then the reaction mixture was allowed to cool down to room temperature and was slowly poured into cold water. The desired 1-acetyl-2-thiohydantoin was then isolated as detailed hereinafter.

General Procedure 3b: Imidazoline Formation from Amino Acid—Step 2—Deacetylation

A 1-acetyl-2-thiohydantoin (1.0 equiv) was suspended in 3 N aqueous HCl (0.2 M). The reaction was heated to 100° C. for 1-18 h. Then the reaction mixture was allowed to cool down to 25° C. and extracted with EtOAc. The combined organic layers were washed with water, dried over magnesium sulfate and concentrated to dryness to obtain the corresponding 2-thiohydantoin. The product was further purified when necessary.

General Procedure 3c: Imidazoline Formation from Amino Acid—Step 3—Reduction

To a solution of LiAlH₄1 M in THE (2.0 equiv) in THE (0.3 M) was added aluminum trichloride (2.5 equiv). The reaction mixture was stirred at 0° C. for 1 h, then a 2-thiohydantoin (1.0 equiv) was added and the mixture was stirred at 0 to 25° C. for 18 h. The reaction mixture was hydrolyzed at 0° C. with an aqueous saturated NaHCO₃ solution and optionally filtered on a Buchner funnel. The aqueous layer was extracted twice with EtOAc. The combined organic layers were washed with brine, dried over magnesium sulfate and concentrated to dryness. The crude was purified as detailed hereinafter.

General Procedure 4a: Diamine Formation—Boc Protection

At 0° C. to a solution of an amine (1.0 equiv) in DCM (0.05 M) was added a solution of Boc₂O (1.5 equiv) in DCM (0.05 M) (if the amine was a salt, triethylamine (1.0 equiv in case of a mono-salt, 2.0 equiv in case of a di-salt) was added beforehand). The reaction mixture was stirred at 0° C. to 25° C. for 15 min to 4 h, then hydrolyzed with an aqueous saturated NaHCO₃ solution and extracted twice with DCM. The combined organic layers were filtered through a hydrophobic cartridge and concentrated to dryness. The crude was purified as detailed hereinafter.

General Procedure 4b: Diamine Formation—Boc Protection

To a solution of an amine (1.0 equiv) in THE (0.1 M) was added Boc₂O (2.0 equiv) and DMAP (1.0 equiv). The reaction mixture was stirred at 25° C. for 16 h, then hydrolyzed with an aqueous saturated NaHCO₃ solution for 10 min and extracted twice with DCM. The combined organic layers were filtered through a hydrophobic cartridge and concentrated to dryness. The crude was purified as detailed hereinafter.

General Procedure 4c: Diamine Formation—Nitrile Reduction

To a solution of a nitrile derivative (1.0 equiv) in MeOH (0.1 M) was added Boc₂O (2.0-3.0 equiv) and freshly crushed cobalt (II) chloride hexahydrate (1.0 equiv). The resulting solution was stirred for 15 min at 25° C. then it was cooled down to −78° C. and sodium tetrahydroborate (3.0 equiv) was added. The reaction mixture was stirred for 18 h while being allowed to slowly warm up to rt. It was then hydrolyzed with water for 10 min, optionally insolubles were removed by Buchner filtration, then the mixture was extracted with EtOAc, washed with brine, dried over magnesium sulfate, and concentrated to dryness. The crude was purified as detailed hereinafter.

General Procedure 4d: Diamine Formation—Buchwald Coupling

To a suspension of a halogen derivative (1.0 equiv) and tert-butyl carbamate (1.2 equiv) in dioxane (0.1 M) was added cesium carbonate (1.4 equiv). The mixture was sparged with argon for 10 min and XPhos Pd G2 (0.1 equiv) was added. The reaction mixture was heated to 100° C. for 1-18 h, then filtered through a pad of Celite® and washed with DCM. The filtrate was concentrated to dryness and purified as detailed hereinafter.

General Procedure 4e: Diamine Formation—Buchwald Coupling

To a suspension of a halogen derivative (1.0 equiv) and tert-butyl carbamate (1.2 equiv) in dioxane (0.3 M) was added cesium carbonate (1.5 equiv). The mixture was sparged with argon for 10 min and XPhos Pd G4 (0.1 equiv) was added. The reaction mixture was heated to 80° C. for 1-18 h, then hydrolyzed with water and extracted twice with EtOAc. The combined organic layers were washed with brine, dried over magnesium sulfate, and concentrated to dryness. The crude was purified as detailed hereinafter.

General Procedure 4f: Diamine Formation—Boc Deprotection

To a solution of a Boc-protected derivative (1.0 equiv) in DCM (0.2 M) was added trifluoroacetic acid (50 equiv). The reaction was stirred at 25° C. for 15 min to 2 h, then concentrated to dryness, passed through an ISOLUTE® SCX-2 cartridge (MeOH, then NH₃ 3 M in MeOH) and concentrated to dryness to obtain the corresponding deprotected derivative.

General Procedure 4g: Diamine Formation—Cyanation

To a solution of a halogen derivative (1.0 equiv) in DMA (0.2 M) was added Zn(CN)₂ (1.1 equiv). The mixture was sparged with argon for 10 min before addition of Pd-116 (10 mol %). The reaction mixture was heated at 110° C. for 30 min to 2 h, then it was filtered through a pad of Celite® and washed with EtOAc. The filtrate was washed with an aqueous saturated NaHCO₃ solution, with brine, dried over magnesium sulfate and concentrated to dryness. The crude was purified as detailed hereinafter.

General Procedure 4h: Diamine Formation—Buchwald Coupling

To a solution of a halogen derivative (1.0 equiv) in dioxane (0.1 M) was added cesium carbonate (3.0 equiv) and an amine (3.0 equiv). The reaction was sparged with argon for 20 min, then Pd₂dba₃ (5 mol %) and BINAP (10 mol %) were added. The reaction mixture was heated to 100° C. for 18 h, then it was filtered on Celite®. The filtrate was diluted with water and extracted three times with EtOAc. The combined organic layers were washed with brine, dried over sodium sulfate and concentrated to dryness. The crude was purified as detailed hereinafter.

General Procedure 4i: Diamine Formation—Alkylation

To a solution of an amide (1.0 equiv) in THE (0.1 M) was added ^{t}BuOK (1.5 equiv). The reaction mixture was stirred at 25° C. for 15 min then an alkylating agent (1.5 equiv) was added and the reaction mixture was stirred at 25° C. for 1 h to 2 days. It was then hydrolyzed with water and extracted with EtOAc. The organic layer was dried over magnesium sulfate and concentrated to dryness. The crude was purified as detailed hereinafter.

General Procedure 5a: Electrophile Formation—Alkylation

To a suspension of a nucleophile (1.0 equiv) in MeCN (0.2 M) was added potassium carbonate (1.1 equiv, or 2.1-2.5 equiv if the nucleophile is a hydrochloride salt) and an alkylating agent (1.1-1.5 equiv). The reaction was stirred at 25° C. for 18-48 h. Then reaction mixture was filtrated, rinsed with MeCN and the filtrate was concentrated to dryness. The residue was further purified when necessary.

General Procedure 5b: Electrophile Formation—TBDMS Deprotection

To a solution of a silylated derivative (1.0 equiv) in MeOH (0.2 M) was added HCl 4 M in dioxane (2.5 equiv). The reaction was stirred at 25° C. for 1.5-3 h and concentrated to dryness to afford the corresponding deprotected compound. The product was further purified when necessary.

General Procedure 5c: Electrophile Formation—Chlorination

At 0° C., to a suspension of an alcohol (1.0 equiv) in DCM (0.2 M) was added DMF (0.1 equiv) and thionyl chloride (10.0 equiv). The reaction was stirred at 25° C. for 18 h and was then concentrated to dryness and co-evaporated thrice with toluene to obtain the desired chlorinated compound. The product was further purified when necessary.

General Procedure 5d: Electrophile Formation—Iodination

To a solution of an alcohol (1.0 equiv) in DCM (0.1 M) was added 1H-imidazole (1.4 equiv, or 2.4 equiv if the acohol was a hydrochloride salt) and triphenylphosphine (1.2 equiv). At 0° C., diiodine (1.3 equiv) was added portionwise and the reaction mixture was stirred at rt for 2 d. In case of uncomplete conversion 1H-imidazole (1.0 equiv), triphenylphosphine (1.0 equiv) and diiodine (1.0 equiv) were added and the reaction mixture was stirred at rt for 2 h. The reaction mixture was filtered and the solid was washed twice with DCM. The filtrate was concentrated to dryness and purified as detailed hereinafter.

General Procedure 5e: Electrophile Formation

To a solution of an alcohol (1.0 equiv) in DCM (0.2 M) was added triethylamine (1.5 equiv) and methanesulfonyl chloride (1.2 equiv). The reaction mixture was stirred at rt for 1-3 h. The mixture was then then hydrolyzed with water, extracted twice with DCM, washed with brine, dried over magnesium sulfate or filtered through a hydrophobic cartridge, then concentrated to dryness. The product was further purified when necessary.

General Procedure 5f: Electrophile Formation—Mesylation

To a solution of an alcohol (1.0 equiv) in DCM (0.2 M) was added triethylamine (1.1 equiv) and methanesulfonyl chloride (1.1 equiv). The reaction mixture was stirred at rt for 1 h. In case of uncomplete conversion, triethylamine (0.5 equiv) and methanesulfonyl chloride (0.5 equiv) were added and the reaction mixture was stirred at rt for 30 min. The mixture was concentrated to dryness and co-evaporated twice with toluene. The product was further purified when necessary.

General Procedure A: Alkylation of Thioureas to Afford Examples

A suspension of electrophile (1.0-15.0 equiv), thiourea (1.0-5.0 equiv) and optionally sodium iodide (1.0-15.0 equiv) in a solvent (C=0.2 M) was heated at 50-120° C. for 16 h to 7 days. The product was isolated as detailed hereinafter.

General Procedure B: Alkylation of Thioureas Followed by a Dehydration Step to Afford Examples

Step 1: A suspension of electrophile (1.0-3.0 equiv), thiourea (1.0-1.5 equiv) in a solvent (C=0.2 M) was heated at 80-110° C. for 2-18 h. The precipitate was isolated by filtration.

Step 2: The resulting solid was suspended in HCl 4 M in dioxane (10 equiv) and was heated at 80-110° C. for 16 h to 6 days. The product was isolated as detailed hereinafter.

General Procedure C: Alkylation of Thioureas in Presence of a Base

At 0° C. to rt, to a solution of a thiourea (1.0-2.0 equiv) in a solvent (0.2 M) was added a base (1.0-2.0 equiv). The mixture was stirred for 5 to 15 min, and then an electrophile (1.0-3.0 equiv) was added. The reaction was stirred at 25° C. to 70° C. for 0.5-18 h. The product was isolated as detailed hereinafter.

Synthesis of Exemplary Compounds of the Invention

Intermediates: Thioureas

### Intermediate 1: (3aR,7aR)-octahydro-2H-benzo[d]imidazole-2-thione

Intermediate 1 was isolated as a yellow solid (127 mg, 62%) according to general procedure 2a, starting from (1R,2R)-cyclohexane-1,2-diamine (150 mg) after purification by flash chromatography (CyHex 100% to CyHex/EtOAc 6:4). M/Z (M+H)⁺: 157.1.

### Intermediate 2: 4-butylimidazolidine-2-thione

Intermediate 2 was isolated as a yellow solid (248 mg, 73%) according to general procedure 2a, starting from hexane-1,2-diamine (250 mg) after purification by flash chromatography (CyHex 100% to CyHex/EtOAc 6:4). M/Z (M+H)⁺: 159.2.

### Intermediate 3: 4-benzylimidazolidine-2-thione

Intermediate 3 was isolated as a pale-yellow solid (407 mg, 64%) according to general procedure 2a, starting from 3-phenylpropane-1,2-diamine (500 mg) after trituration in DCM and Et₂O. M/Z (M+H)⁺: 193.1.

### Intermediate 4: 6-chloro-3,4-dihydroquinazoline-2(1-H)-thione

Intermediate 4 was isolated as a yellow solid (537 mg, 85%) according to general procedure 2b, starting from 2-(aminomethyl)-4-chloroaniline (500 mg). M/Z (M³⁵[Cl]+H)⁺: 199.0.

### Intermediate 5: 3,4-dihydroquinazoline-2(1H)-thione

Intermediate 5 was isolated as a white solid (970 mg, 72%) according to general procedure 2b, starting from 2-(aminomethyl)aniline (1.00 g). M/Z (M+H)⁺: 165.1.

### Intermediate 6: 7-chloro-3,4-dihydroquinazoline-2(1H)-thione

Intermediate 6 was isolated as a white solid (300 mg, 69%) according to general procedure 2c, starting from 2-(aminomethyl)-5-chloroaniline dihydrochloride (500 mg). M/Z (M³⁵[Cl]+H)⁺: 199.1.

### Intermediate 7: 1,2,4,5-tetrahydro-3H-benzo[e][1,3]diazepine-3-thione

Intermediate 7 was isolated as a white solid (460 mg, 70%) according to general procedure 2b, starting from 2 1,2-phenylenedimethanamine (500 mg). M/Z (M+H)⁺: 179.1.

### Intermediate 8: 4-(4-chlorophenyl)imidazolidine-2-thione

Intermediate 8 was isolated as a white solid (300 mg, 69%) according to general procedure 2c, starting from 1-(4-chlorophenyl)ethane-1,2-diamine dihydrochloride (500 mg) after trituration in cold DCM (5 mL). M/Z (M[³⁵Cl]+H)⁺: 213.1.

### Intermediate 9: 4-cyclohexylimidazolidine-2-thione

Intermediate 9 was isolated as a white solid (445 mg, 69%) according to general procedure 2a, starting from 1-cyclohexylethane-1,2-diamine (500 mg) after purification by flash chromatography (20 μm, CyHex 100% to EtOAc 100%). M/Z (M+H)⁺: 185.1.

### Intermediate 10: (4S,5S)-4,5-diphenylimidazolidine-2-thione

Intermediate 10 was isolated as an off-white solid (500 mg, 84%) according to general procedure 2a, starting from 1-(1S,2S)-1,2-diphenylethane-1,2-diamine (500 mg) after purification by flash chromatography (CyHex 100% to CyHex/EtOAc 20:80). M/Z (M+H)⁺: 255.1.

### Intermediate 11: 5-fluoro-3,4-dihydroquinazoline-2(1H)-thione

Intermediate 11 was isolated as a white solid (290 mg, 45%) according to general procedure 2b, starting from 2-(aminomethyl)-3-fluoroaniline (500 mg) after purification by flash chromatography (CyHex 100% to EtOAc 100%). M/Z (M+H)⁺: 183.1.

### Intermediate 12: 4,4-dimethyl-3,4-dihydroquinazoline-2(1H)-thione

Intermediate 12 was isolated as a white solid (340 mg, 53%) according to general procedure 2b, starting from 2-(2-aminopropan-2-yl)aniline (500 mg). M/Z (M+H)⁺: 193.1.

### Intermediate 13: 1,3,4,5-tetrahydro-2H-benzo[d][1,3]diazepine-2-thione

Intermediate 13 was isolated as a white solid (475 mg, 73%) according to general procedure 2b, starting from 2-(2-aminoethyl)aniline (500 mg). M/Z (M+H)⁺: 178.8.

### Intermediate 14: 4-phenyl-3,4-dihydroquinazoline-2(1H)-thione

Intermediate 14 was isolated as a white solid (445 mg, 73%) according to general procedure 2b, starting from 2-(amino(phenyl)methyl)aniline (500 mg). M/Z (M+H)⁺: 241.0.

### Intermediate 15: 3,4-dihydropyrido[2,3-d]pyrimidine-2(1H)-thione

Intermediate 15 was isolated as a beige solid (390 mg, 73%) according to general procedure 2b, starting from 2-(amino(phenyl)methyl)aniline (500 mg) after trituration in hot DCE (15 mL) for 4 h. M/Z (M+H)⁺: 166.1.

### Intermediate 16: 4-methyl-4-phenylimidazolidine-2-thione

Intermediate 16 was isolated as a white solid (555 mg, 87%) according to general procedure 2a, starting from 2-phenylpropane-1,2-diamine (500 mg) after purification by flash chromatography (CyHex/EtOAc 8:2 to CyHex/EtOAc 5:5). M/Z (M+H)⁺: 192.9.

### Intermediate 17: 4-(4-methoxybenzyl)-4-methylimidazolidine-2-thione

Intermediate 17 was isolated as a colorless oil (550 mg, 90%) according to general procedure 2a, starting from 3-(4-methoxyphenyl)-2-methylpropane-1,2-diamine (500 mg) after purification by flash chromatography (CyHex/EtOAc 9:1 to EtOAc 100%). M/Z (M+H)⁺: 237.0.

### Intermediate 18: 1-butylimidazolidine-2-thione

Intermediate 18 was isolated as a white solid (430 mg, 63%) according to general procedure 2a, starting from 1-butylethane-1,2-diamine (500 mg) after purification by flash chromatography (CyHex/EtOAC 9:1 to EtOAC 100%). M/Z (M+H)⁺: 158.9.

### Intermediate 19: 1-benzylimidazolidine-2-thione

At 0° C., to a solution of N¹-benzylethane-1,2-diamine (500 mg, 1.0 equiv) in DCM (17 mL) was added di(1H-imidazol-1-yl)methanethione (593 mg, 1.0 equiv). The reaction was stirred at 5° C. for 2 h and concentrated to dryness. The residue was triturated in DCM (5 mL) for 30 min, then the precipitate was filtered and washed with DCM to obtain a white solid (380 mg, 59%). M/Z (M+H)⁺: 192.9.

### Intermediate 20: 1-isopropylimidazolidine-2-thione

Intermediate 20 was isolated as a pale yellow solid (460 mg, 65%) according to general procedure 2a, starting from N¹-isopropylethane-1,2-diamine (500 mg) after purification by flash chromatography (CyHex 100% to CyHex/EtOAc 2:8). M/Z (M+H)⁺: 144.9.

### Intermediate 21: hexahydroimidazo[1,5-a]pyridine-3(2H)-thione

Intermediate 21 was isolated as a white solid (640 mg, 94%) according to general procedure 2a, starting from piperidin-2-ylmethanamine (500 mg) after purification by flash chromatography (CyHex/EtOAC 9:1 to EtOAC 100%). M/Z (M+H)⁺: 156.9.

### Intermediate 22: 1-acetyl-5-(thiophen-2-ylmethyl)-2-thioxoimidazolidin-4-one

Intermediate 22 was isolated as a pale yellow solid (383 mg, 52%) according to general procedure 3a, starting from 2-amino-3-(thiophen-2-yl)propanoic acid (500 mg) after filtration of the reaction mixture, washing of the solid with water, and purification by flash chromatography (CyHex 100% to CyHex/EtOAc 85:15). M/Z (M+H-Ac)⁺: 212.9.

### Intermediate 23: 5-(thiophen-2-ylmethyl)-2-thioxoimidazolidin-4-one

Intermediate 23 was isolated as a yellow solid (291 mg, 91%) according to general procedure 3b, starting from intermediate 22 (383 mg). M/Z (M+H)⁺: 212.8.

### Intermediate 24: 4-(thiophen-2-ylmethyl)imidazolidine-2-thione

Intermediate 24 was isolated as a white solid (172 mg, 63%) according to general procedure 3c, starting from intermediate 23 (291 mg) after purification by flash chromatography (20 μm, CyHex 100% to CyHex/EtOAc 50:50). M/Z (M+H)⁺: 198.9.

### Intermediate 25: 7-bromo-3,4-dihydroquinazoline-2(1H)-thione

Intermediate 25 was isolated according to general procedure 2b, starting from 2-(aminomethyl)-5-bromoaniline (500 mg). The obtained solid was dissolved in DCM (30 mL), washed with water (2×20 mL), filtered through a hydrophobic cartridge and concentrated to dryness. The resulting solid was triturated overnight in MeOH (40 mL) to afford a beige solid (440 mg, 73%). M/Z (M[⁷⁹Br]+H)⁺: 242.8.

### Intermediate 26: 6-bromo-3,4-dihydroquinazoline-2(1H)-thione

Intermediate 26 was isolated according to general procedure 2d, starting from 2-(aminomethyl)-4-bromoaniline dihydrochloride (500 mg). The obtained residue was triturated in DCM (5 mL) at 25° C. for 30 min, then the precipitate was filtered and washed with DCM. The resulting solid was dissolved in DCM (30 mL), washed with water (2×20 mL), filtered through a hydrophobic cartridge and concentrated to dryness to obtain a beige solid (285 mg, 64%). M/Z (M[⁷⁹Br]+H)⁺: 242.9.

### Intermediate 27: 4,6-diazaspiro[2.4]heptane-5-thione

Intermediate 27 was isolated as a white solid (105 mg, 65%) according to general procedure 2d, starting from 1-(aminomethyl)cyclopropan-1-amine dihydrochloride (200 mg) after purification by flash chromatography (CyHex 100% to EtOAc 100%). M/Z (M+H)⁺: 129.1.

### Intermediate 28: tert-butyl (2-(((tert-butoxycarbonyl)amino)methyl)-3-chlorophenyl)carbamate

Intermediate 28 was isolated as an orange oil (1.69 g, 90%) according to general procedure 4c, starting from 2-amino-6-chlorobenzonitrile (800 mg) after purification by flash chromatography (CyHex 100% to CyHex/EtOAc 90:10). M/Z (M[³⁵Cl]-Boc-^{t}Bu+3H)⁺: 200.9.

### Intermediate 29: 2-(aminomethyl)-3-chloroaniline

Intermediate 29 was isolated as an orange oil (574 mg) according to general procedure 4f, starting from intermediate 28 (1.69 g).

¹H-NMR (DMSO-d₆, 400 MHz) δ: 1.77 (bs, 2H, NH₂); 3.78 (s, 2H, N—CH₂); 5.50 (bs, 2H, NH₂); 6.54-6.59 (m, 2H, 2 Ar); 6.9 (t, J 8.0 Hz, 1H, Ar).

### Intermediate 30: 5-chloro-3,4-dihydroquinazoline-2(1H)-thione

Intermediate 30 was isolated as a beige solid (106 mg, 11% over two steps) according to general procedure 2b, starting from intermediate 29 (574 mg). M/Z (M[³⁵Cl]+H)⁺: 198.9.

### Intermediate 31: tert-butyl (2-cyano-5-fluorophenyl)carbamate

Intermediate 31 was isolated as a white solid (2.05 g, 68%) according to general procedure 4d, starting from 2-chloro-4-fluorobenzonitrile (2.00 g) after purification by flash chromatography (CyHex/DCM 90:10 to DCM 100%). M/Z (M-^{t}Bu+2H)⁺: 180.9.

### Intermediate 32: tert-butyl (2-(((tert-butoxycarbonyl)amino)methyl)-5-fluorophenyl)carbamate

Intermediate 32 was isolated as a white sticky solid (463 mg, 64%) according to general procedure 4c, starting from intermediate 31 (500 mg) after purification by flash chromatography (CyHex 100% to EtOAc 100%). M/Z (M-Boc-^{t}Bu+3H)⁺: 184.9.

### Intermediate 33: 2-(aminomethyl)-5-fluoroaniline

Intermediate 33 was isolated as a yellow oil (266 mg, 96%) according to general procedure 4f, starting from intermediate 32 (676 mg).

¹H-NMR (DMSO-d₆, 400 MHz) δ: 2.07 (bs, 2H, NH₂); 3.58 (s, 2H, N—CH₂); 5.42 (bs, 2H, NH₂); 6.23 (td, J8.5, 2.7 Hz, 1H, Ar); 6.36 (dd, J 11.6, 2.7 Hz, 1H, Ar); 6.96-7.00 (m, 1H, Ar).

### Intermediate 34: 7-fluoro-3,4-dihydroquinazoline-2(1H)-thione

Intermediate 34 was isolated as a beige solid (239 mg, 69%) according to general procedure 2b, starting from intermediate 33 (266 mg). M/Z (M+H)⁺: 182.9.

### Intermediate 35: tert-butyl (2-bromo-5-fluorobenzyl)carbamate

Intermediate 35 was isolated as a colorless liquid (1.35 g, 91%) according to general procedure 4a starting from (2-bromo-5-fluorophenyl)methanamine (1.00 g) after purification by flash chromatography (CyHex 100% to CyHex/EtOAc 80:20). M/Z (M[⁷⁹Br]-^{t}Bu+2H)⁺: 247.8.

### Intermediate 36: tert-butyl (2-bromo-5-fluorobenzyl)(tert-butoxycarbonyl)carbamate

Intermediate 36 was isolated as a white solid (198 mg, 99%) according to general procedure 4b starting from intermediate 35 (150 mg) after purification by flash chromatography (CyHex 100% to CyHex/EtOAc 90:10). ¹H-NMR (DMSO-d₆, 400 MHz) δ: 1.40 (s, 18H, 2 C(CH₃)₃); 4.71 (s, 2H, N—CH₂); 6.81 (dd, J9.7, 3.0 Hz, 1H, Ar); 7.14 (td, J8.6, 3.0 Hz, 1H, Ar); 7.69 (dd, J8.6, 5.3 Hz, 1H, Ar).

### Intermediate 37: tert-butyl (tert-butoxycarbonyl)(2-((tert-butoxycarbonyl)amino)-5-fluorobenzyl)carbamate

Intermediate 37 was isolated as a colorless oil (164 mg, 76%) according to general procedure 4e, starting from intermediate 36 (198 mg) after purification by flash chromatography (CyHex 100% to CyHex/EtOAc 90:10). ¹H-NMR (DMSO-d₆, 400 MHz) δ: 1.31 (s, 18H, 2 C(CH₃)₃); 1.45 (s, 9H, C(CH₃)₃); 4.63 (s, 2H, N—CH₂); 6.72 (dd, J9.7, 3.0 Hz, 1H, Ar); 7.06 (td, J8.6, 3.0 Hz, 1H, Ar); 7.33 (dd, J 8.6, 5.3 Hz, 1H, Ar); 8.76 (bs, 1H, NH).

### Intermediate 38: 2-(aminomethyl)-5-fluoroaniline

Intermediate 38 was isolated as a yellow oil (45 mg, 86%) according to general procedure 4f, starting from intermediate 37 (164 mg).

¹H-NMR (DMSO-d₆, 400 MHz) δ: 1.79 (bs, 2H, NH₂); 3.57 (s, 2H, N—CH₂); 4.90 (bs, 2H, NH₂); 6.57 (dd, J8.6, 5.2 Hz, 1H, Ar); 6.73 (td, J8.6, 3.0 Hz, 1H, Ar); 6.90 (dd, J10.0, 3.0 Hz, 1H, Ar).

### Intermediate 39: 6-fluoro-3,4-dihydroquinazoline-2(1H)-thione

Intermediate 39 was isolated as a beige solid (45 mg, 77%) according to general procedure 2b, starting from intermediate 38 (266 mg). M/Z (M+H)⁺: 182.9.

### Intermediate 40: tert-butyl (2-amino-3-bromobenzyl)carbamate

Intermediate 40 was isolated as a white solid (490 mg) according to general procedure 4c, starting from 2-amino-3-bromobenzonitrile (500 mg) after purification by flash chromatography (CyHex 100% to CyHex/EtOAc 90:10). M/Z (M[⁷⁹Br]-^{t}Bu+2H)⁺: 244.9.

### Intermediate 41: 2-(aminomethyl)-6-bromoaniline

Intermediate 41 was isolated as an orange oil (293 mg) according to general procedure 4f, starting from intermediate 40 (490 mg).

¹H-NMR (DMSO-d₆, 400 MHz) δ: 1.87 (bs, 2H, NH₂); 3.69 (s, 2H, N—CH₂); 5.40 (bs, 2H, NH₂); 6.48 (t, J7.6 Hz, 1H, Ar); 7.04 (d, J7.0 Hz, 1H, Ar); 7.26 (dd, J8.1, 1.5 Hz, 1H, Ar).

### Intermediate 42: 8-bromo-3,4-dihydroquinazoline-2(1H)-thione

Intermediate 42 was isolated as a white solid (188 mg, 31% over 3 steps) according to general procedure 2b, starting from intermediate 41 (246 mg). M/Z (M[⁷⁹Br]+H)⁺: 242.9.

### Intermediate 43: 2-amino-5-chloro-3-fluorobenzonitrile

Intermediate 43 was isolated as a beige solid (639 mg, 84%) according to general procedure 4g, starting from 2-bromo-4-chloro-6-fluoroaniline (1.00 g) after purification by flash chromatography (CyHex 100% to CyHex/EtOAc 90:10).

¹H-NMR (DMSO-d₆, 400 MHz) δ: 6.37 (s, 2H, NH₂); 7.44 (dd, J2.3, 1.5 Hz, 1H, Ar); 7.52 (dd, J11.2, 2.4 Hz, 1H, Ar).

### Intermediate 44: tert-butyl (2-amino-5-chloro-3-fluorobenzyl)carbamate

Intermediate 44 was isolated as a brown oil (873 mg, 72%) according to general procedure 4c, starting from intermediate 43 (756 mg) after purification by flash chromatography (CyHex 100% to CyHex/EtOAc 85:15). M/Z (M-^{t}Bu+2H)⁺: 219.0.

### Intermediate 45: 2-(aminomethyl)-4-chloro-6-fluoroaniline

Intermediate 45 was isolated as a brown pale solid (390 mg, 70%) according to general procedure 4f, starting from intermediate 44 (873 mg).

¹H-NMR (DMSO-d₆, 400 MHz) δ: 1.90 (bs, 2H, NH₂); 3.64 (s, 2H, N—CH₂); 5.21 (bs, 2H, NH₂); 6.99-7.01 (m, 1H, Ar); 7.05 (dd, J6.9, 2.5 Hz, 1H, Ar).

### Intermediate 46: 6-chloro-8-fluoro-3,4-dihydroquinazoline-2(1H)-thione

Intermediate 46 was isolated as a white solid (417 mg, 86%) according to general procedure 2b, starting from intermediate 45 (390 mg). M/Z (M[³⁵Cl]+H)⁺: 216.8.

### Intermediate 47: 2-(butylamino)benzonitrile

Intermediate 47 was isolated as a yellow oil (416 mg, 97%) according to general procedure 4h, starting from 2-bromobenzonitrile (450 mg) after purification by flash chromatography (CyHex 100% to CyHex/DCM 80:20). M/Z (M+H)⁺: 175.0.

### Intermediate 48: tert-butyl (2-(butylamino)benzyl)carbamate

Intermediate 48 was isolated as a yellow oil (313 mg) according to general procedure 4c, starting from intermediate 47 (560 mg) after purification by flash chromatography (CyHex 100% to CyHex/EtOAc 0:100).

### Intermediate 49: 2-(aminomethyl)-N-butylaniline

Intermediate 49 was isolated as a green oil (190 mg) according to general procedure 4f, starting from intermediate 48 (313 mg).

¹H-NMR (DMSO-d₆, 400 MHz) δ: 0.94 (t, J7.5 Hz, 3H, CH₃); 1.36-1.45 (m, 2H, CH₂); 1.56-1.64 (m, 2H, CH₂); 3.06 (t, J7.2 Hz, 2H, N—CH₂-Et); 3.97 (s, 2H, N—CH₂—Ar); 5.25 (bs, 1H, NH); 6.62-6.66 (m, 2H, 2 Ar); 7.14-7.22 (m, 2H, 2Ar); 7.91 (bs, 2H, NH₂).

### Intermediate 50: 1-butyl-3,4-dihydroquinazoline-2(1H)-thione

Intermediate 50 was isolated as a white solid (107 mg, 15% over 3 steps) according to general procedure 2e, starting from intermediate 49 (190 mg) after purification by flash chromatography (20 μm, CyHex 100% to CyHex/EtOAc 60:40). M/Z (M+H)⁺: 221.0.

### Intermediate 51: tert-butyl (2-bromobenzyl)carbamate

Intermediate 51 was isolated as a colorless oil (578 mg, 90%) according to general procedure 4a starting from (2-bromophenyl)methanamine hydrochloride (500 mg) after purification by flash chromatography (CyHex 100% to CyHex/EtOAc 85:15). M/Z (M[⁷⁹Br]-^{t}Bu+2H)⁺: 229.9.

### Intermediate 52: tert-butyl (2-bromobenzyl)(butyl)carbamate

Intermediate 52 was isolated as a colorless oil (92 mg, 77%) according to general procedure 4i, starting from intermediate 51 (100 mg) and 1-bromobutane (57 μL) after purification by flash chromatography (CyHex 100% to CyHex/EtOAc 90:10). M/Z (M[⁷⁹Br]-^{t}Bu+2H)⁺: 286.0.

### Intermediate 53: tert-butyl (2-((tert-butoxycarbonyl)amino)benzyl)(butyl)carbamate

Intermediate 53 was isolated as a brown pale oil (399 mg) according to general procedure 4e, starting from intermediate 52 (425 mg) after purification by flash chromatography (CyHex 100% to DCM 100%).

¹H-NMR (DMSO-d₆, 400 MHz) δ: 0.84 (t, J7.3 Hz, 3H, CH₃); 1.19 (q, J7.3 Hz, 2H, CH₂); 1.31-1.41 (m, 11H, CH₂+C(CH₃)₃); 1.45 (s, 9H, C(CH₃)₃); 3.06 (t, J7.3 Hz, 2H, N—CH₂-Et); 4.35 (s, 2H, N—CH₂—Ar); 7.06-7.09 (m, 1H, Ar); 7.14-7.18 (m, 1H, Ar); 7.21-7.25 (m, 1H, Ar); 7.64 (bs, 1H, Ar); 8.69 (bs, 1H, NH).

### Intermediate 54: 2-((butylamino)methyl)aniline

Intermediate 54 was isolated as a pale-brown oil (166 mg) according to general procedure 4f, starting from intermediate 53 (399 mg). M/Z (M+H)⁺: 179.1.

### Intermediate 55: 3-butyl-3,4-dihydroquinazoline-2(1H)-thione

Intermediate 55 was isolated as a white solid (92 mg, 34% over 3 steps) according to general procedure 2e, starting from intermediate 54 (166 mg) after purification by flash chromatography (CyHex 100% to CyHex/EtOAc 85:15). M/Z (M+H)⁺: 221.1.

### Intermediate 56: 1-acetyl-5-(4-chlorobenzyl)-2-thioxoimidazolidin-4-one

Intermediate 56 was isolated as a white solid (269 mg, 76%) according to general procedure 3a, starting from 2-amino-3-(4-chlorophenyl)propanoic acid (250 mg) after filtration of the reaction mixture and washing of the solid with water. M/Z (M+H-Ac)⁺: 240.9.

### Intermediate 57: 5-(4-chlorobenzyl)-2-thioxoimidazolidin-4-one

Intermediate 57 was isolated as a white solid (190 mg, 83%) according to general procedure 3b, starting from intermediate 56 (269 mg). M/Z (M[³⁵Cl]+H)⁺: 241.0.

### Intermediate 58: 4-(4-chlorobenzyl)imidazolidine-2-thione

Intermediate 58 was isolated as a white solid (70 mg, 38%) according to general procedure 3c, starting from intermediate 57 (190 mg) after purification by flash chromatography (20 μm, CyHex/EtOAc 90:10 to CyHex/EtOAc 50:50). M/Z (M+[³⁵Cl])⁺: 227.1.

### Intermediate 95: (S)-5-((1H-indol-3-yl)methyl)-1-acetyl-2-thioxoimidazolidin-4-one

Intermediate 95 was isolated as a white solid (211 mg, 58%) according to general procedure 3a, starting from D-tryptophan (250 mg) after extraction with EtOAc (2×10 mL), washing with water (10 mL), brine (10 mL) and drying over magnesium sulfate followed by concentration to dryness and purification by flash chromatography (CyHex 100% to CyHex/EtOAc 60:40). M/Z (M+H)⁺: 288.1.

### Intermediate 96: (S)-5-((1H-indol-3-yl)methyl)-2-thioxoimidazolidin-4-one

Intermediate 96 was isolated as a brown oil (306 mg) according to general procedure 3b, starting from intermediate 95 (307 mg, 1.07 mmol). M/Z (M+H)⁺: 245.9.

### Intermediate 97: (S)-4-((1H-indol-3-yl)methyl)imidazolidine-2-thione

Intermediate 97 was isolated as a yellow hygroscopic solid (43 mg) according to general procedure 3c, starting from intermediate 96 (1.07 mmol) after purification by flash chromatography (20 μm, CyHex/EtOAc 90:10 to CyHex/EtOAc 40:60). M/Z (M+H)⁺: 232.1.

### Intermediate 98: (S)-1-acetyl-5-(3-chlorobenzyl)-2-thioxoimidazolidin-4-one

Intermediate 98 was isolated as a white solid (532 mg, 75%) according to general procedure 3a, starting from (S)-2-amino-3-(3-chlorophenyl)propanoic acid (500 mg) after filtration of the reaction mixture and washing of the solid with water. M/Z (M[³⁵Cl]+H-Ac)⁺: 240.9.

### Intermediate 99: (S)-5-(3-chlorobenzyl)-2-thioxoimidazolidin-4-one

Intermediate 99 was isolated as a white solid (383 mg, 85%) according to general procedure 3b, starting from intermediate 98 (532 mg). M/Z (M[³⁵Cl]+H)⁺: 241.0.

### Intermediate 100: (S)-4-(3-chlorobenzyl)imidazolidine-2-thione

Intermediate 100 was isolated as a white solid (92 mg, 26%) according to general procedure 3c, starting from intermediate 99 (383 mg) after purification by flash chromatography (20 μm, CyHex 100% to CyHex/EtOAc 50:50). M/Z (M[³⁵Cl]+H)⁺: 226.9.

### Intermediate 101: 1-acetyl-5-(3-methylbenzyl)-2-thioxoimidazolidin-4-one

Intermediate 101 was isolated as a yellow solid (486 mg, 66%) according to general procedure 3a, starting from 2-amino-3-(m-tolyl)propanoic acid (500 mg) after filtration of the reaction mixture and washing of the solid with water. M/Z (M+H-Ac)⁺: 220.9

### Intermediate 102: 5-(3-methylbenzyl)-2-thioxoimidazolidin-4-one

Intermediate 102 was isolated as a white solid (367 mg, 90%) according to general procedure 3b, starting from intermediate 101 (486 mg). M/Z (M+H)⁺: 221.0.

### Intermediate 103: 4-(3-methylbenzyl)imidazolidine-2-thione

Intermediate 103 was isolated as a yellow solid (120 mg, 35%) according to general procedure 3c, starting from intermediate 102 (367 mg) after purification by flash chromatography (20 μm, CyHex 100% to CyHex/EtOAc 50:50). M/Z (M+H)⁺: 206.9.

### Intermediate 104: 4-methyl-3,4-dihydroquinazoline-2(1H)-thione

Intermediate 104 was isolated as a pale yellow solid (583 mg, 89%) according to general procedure 2e, starting from 2-(1-aminoethyl)aniline (500 mg) after purification by flash chromatography (CyHex100% to CyHex/EtOAc 50:50). M/Z (M+H)⁺: 179.0.

### Intermediate 105: tert-butyl (2-cyano-3-fluorophenyl)carbamate

Intermediate 105 was isolated as a white solid (91 mg, 77%) according to general procedure 4e, starting from 2-bromo-6-fluorobenzonitrile (100 mg) after purification by flash chromatography (CyHex/DCM 100:0 to DCM 100%). M/Z (M-^{t}Bu+H)⁺: 180.9.

### Intermediate 106: tert-butyl (2-(((tert-butoxycarbonyl)amino)methyl)-3-fluorophenyl)carbamate

Intermediate 106 was isolated as a white hygroscopic solid (389 mg, 39%) according to general procedure 4c, starting from intermediate 105 (695 mg) after purification by flash chromatography (CyHex 100% to CyHex/EtOAc 80:20). M/Z (M+Na)⁺: 363.3.

### Intermediate 107: 2-(aminomethyl)-3-fluoroaniline

Intermediate 107 was isolated as a yellow oil (155 mg, 97%) according to general procedure 4f, starting from intermediate 106 (389 mg).

¹H-NMR (DMSO-d₆, 400 MHz) δ: 1.88 (bs, 2H, NH₂); 3.66 (d, J1.8 Hz, 2H, CH₂); 5.48 (bs, 2H, NH₂); 6.27 (ddd, J9.7, 8.1, 1.0 Hz, 1H, Ar); 6.40-6.45 (m, 1H, Ar); 6.87-6.92 (m, 1H, Ar).

### Intermediate 108: 5-fluoro-3,4-dihydroquinazoline-2(1H)-thione

Intermediate 108 was isolated as a white solid (74 mg, 38%) according to general procedure 4b starting from intermediate 107 (150 mg). M/Z (M+H)⁺: 182.9.

### Intermediate 109: 1-acetyl-5-(3-fluorobenzyl)-2-thioxoimidazolidin-4-one

Intermediate 109 was isolated as a yellow solid (631 mg, 87%) according to general procedure 3a, starting from 2-amino-3-(3-fluorophenyl)propanoic acid (500 mg) after filtration of the reaction mixture and washing of the solid with water. M/Z (M+2H—Ac)⁺: 224.9.

### Intermediate 110: 5-(3-fluorobenzyl)-2-thioxoimidazolidin-4-one

Intermediate 110 was isolated as a white solid (450 mg, 85%) according to general procedure 3b, starting from intermediate 109 (631 mg). M/Z (M+H)⁺: 225.0.

### Intermediate 111: 4-(3-fluorobenzyl)imidazolidine-2-thione

Intermediate 111 was isolated as a white solid (87 mg, 21%) according to general procedure 3c, starting from intermediate 110 (450 mg) after purification by flash chromatography (CyHex/EtOAc 90:10 to CyHex/EtOAc 40:60). M/Z (M+H)⁺: 211.0.

### Intermediate 112: 1-acetyl-5-(4-methylbenzyl)-2-thioxoimidazolidin-4-one

Intermediate 112 was isolated as a yellow solid (745 mg) according to general procedure 3a, starting from 2-amino-3-(p-tolyl)propanoic acid (500 mg, 2.79 mmol) after filtration of the reaction mixture and washing of the solid with water. M/Z (M+2H—Ac)⁺:221.0.

### Intermediate 113: 5-(4-methylbenzyl)-2-thioxoimidazolidin-4-one

Intermediate 113 was isolated as a white solid (430 mg, 70% over 2 steps) according to general procedure 3b, starting from intermediate 112 (2.79 mmol). M/Z (M+H)⁺: 221.0.

### Intermediate 114: 4-(4-methylbenzyl)imidazolidine-2-thione

Intermediate 114 was isolated as a white solid (116 mg, 29%) according to general procedure 3c, starting from intermediate 113 (430 mg) after purification by flash chromatography (CyHex/EtOAc 90:10 to CyHex/EtOAc 40:60). M/Z (M+H)⁺: 207.0

### Intermediate 115: 1-acetyl-5-(2-chlorobenzyl)-2-thioxoimidazolidin-4-one

Intermediate 115 was isolated as a yellow solid (699 mg, 99%) according to general procedure 3a, starting from 2-amino-3-(2-chlorophenyl)propanoic acid (500 mg) after filtration of the reaction mixture and washing of the solid with water. M/Z (M[³⁵Cl]+2H—Ac)⁺: 241.0.

### Intermediate 116: 5-(2-chlorobenzyl)-2-thioxoimidazolidin-4-one

Intermediate 116 was isolated as a white solid (421 mg, 71%) according to general procedure 3b, starting from intermediate 115 (699 mg). M/Z (M[³⁵Cl]+H)⁺: 241.0.

### Intermediate 117: 4-(2-chlorobenzyl)imidazolidine-2-thione

Intermediate 117 was isolated as a white solid (66 mg, 17%) according to general procedure 3c, starting from intermediate 116 (421 mg) after two purifications by flash chromatography (CyHex/EtOAc 90:10 to CyHex/EtOAc 50:50, then CyHex/EtOAc 80:20 to CyHex/EtOAc 50:50). M/Z (M[³⁵Cl]+H)⁺: 227.0.

### Intermediate 118: (R)-1-acetyl-5-(4-methoxybenzyl)-2-thioxoimidazolidin-4-one

Intermediate 118 was isolated as a yellow solid (533 mg, 75%) according to general procedure 3a, starting from (R)-2-amino-3-(4-methoxyphenyl)propanoic acid (500 mg) after filtration of the reaction mixture and washing of the solid with water. M/Z (M+2H—Ac)⁺: 237.0.

### Intermediate 119: (R)-5-(4-methoxybenzyl)-2-thioxoimidazolidin-4-one

Intermediate 119 was isolated as a yellow solid (360 mg, 80%) according to general procedure 3b, starting from intermediate 118 (533 mg). M/Z (M+H)⁺: 237.1.

### Intermediate 120: (R)-4-(4-methoxybenzyl)imidazolidine-2-thione

Intermediate 120 was isolated as a white solid (106 mg, 31%) according to general procedure 3c, starting from intermediate 119 (360 mg) after purification by flash chromatography (CyHex/EtOAc 90:10 to CyHex/EtOAc 50:50). M/Z (M+H)⁺: 223.0.

### Intermediate 121: 1-acetyl-5-phenethyl-2-thioxoimidazolidin-4-one

Intermediate 121 was isolated as a yellow solid (607 mg, 83%) according to general procedure 3a, starting from 2-amino-4-phenylbutanoic acid (500 mg) after filtration of the reaction mixture and washing of the solid with water. M/Z (M+2H—Ac)⁺: 221.0.

### Intermediate 122: 5-phenethyl-2-thioxoimidazolidin-4-one

Intermediate 122 was isolated as a beige solid (443 mg, 83%) according to general procedure 3b, starting from intermediate 121 (607 mg). M/Z (M+H)⁺: 221.0.

### Intermediate 123: 4-phenetylimidazolidine-2-thione

Intermediate 123 was isolated as a white solid (122 mg) according to general procedure 3c, starting from intermediate 122 (443 mg) after three purifications by flash chromatography (CyHex/EtOAc 90:10 to CyHex/EtOAc 50:50, then CyHex/EtOAc 80:20 to CyHex/EtOAc 40:60 and then 20 μm, CyHex/EtOAc 90:10 to CyHex/EtOAc 50:50). M/Z (M+H)⁺: 207.0.

### Intermediate 124: 1-acetyl-5-(4-fluorobenzyl)-2-thioxoimidazolidin-4-one

Intermediate 124 was isolated as a yellow solid (547 mg, 75%) according to general procedure 3a, starting from 2-amino-3-(4-fluorophenyl)propanoic acid (500 mg) after filtration of the reaction mixture and washing of the solid with water. M/Z (M+2H—Ac)⁺: 225.0.

### Intermediate 125: 5-(4-fluorobenzyl)-2-thioxoimidazolidin-4-one

Intermediate 125 was isolated as a yellow solid (482 mg) according to general procedure 3b, starting from intermediate 124 (547 mg, 2.05 mmol). M/Z (M+H)⁺: 225.0

### Intermediate 126: 4-(4-fluorobenzyl)imidazolidine-2-thione

Intermediate 126 was isolated as a white solid (136 mg, 32% over 2 steps) according to general procedure 3c, starting from intermediate 125 (2.05 mmol) after purification by flash chromatography (CyHex 90:10 to CyHex/EtOAc 20:80). M/Z (M+H)⁺: 211.0.

### Intermediate 127: (3,4,5-triiodophenyl)methanamine

To a cloudy solution of (3,4,5-triiodophenyl)methanol (750 mg, 1.0 equiv) in THE (7.5 mL) was added diphenyl phosphorazidate (498 μL, 1.5 equiv) and DBU (346 μL, 1.5 equiv). The reaction was stirred at 25° C. for 24 h. Then triphenylphosphine (688 mg, 1.7 equiv) was added in one portion followed by water (2.78 mL, 100 equiv). After 20 min, when gaz evolution stopped, the reaction mixture was subjected to microwave irradiation at 80° C. for 10 min. The reaction mixture was diluted with EtOAc (75 mL), washed with water (3×75 mL). The combined aqueous layers were extracted with EtOAc (3×100 mL), and the resulting organics layers were with washed brine (100 mL), dried over sodium sulfate and concentrated to dryness. The crude was taken up in EtOAc (5 mL), triturated and filtrated to obtain a white solid (425 mg, 57%). M/Z (M+H)⁺: 485.7.

### Intermediate 128: 2,2,2-trifluoro-N-(3,4,5-triiodobenzyl)acetamide

At 0° C., trifluoroacetic anhydride (8 mL) was added dropwise on intermediate 127 (850 mg, 1.0 equiv) and the reaction mixture was stirred at 0° C. for 15 min, then at 25° C. for 2.5 h. At 0° C. the mixture was hydrolyzed by dropwise addition of water, then the precipitate was filtrated and washed with water to obtain a white solid (968 mg, 95%). ¹H-NMR (DMSO-d₆, 400 MHz) δ: 4.24 (d, J5.9 Hz, 2H, N—CH₂); 7.82 (s, 2H, 2 Ar); 9.42 (t, J5.4 Hz, 1H, NH).

### Intermediate 129: 2,2,2-trifluoro-N-(3,4,5-triiodo-2-nitrobenzyl)acetamide

At 0° C., to solid intermediate 128 (505 mg, 1.0 equiv) was added dropwise nitric acid (5 mL). The resulting orange solution was stirred at 0° C. for 30 min and then poured into ice-cold water (50 mL). The formed precipitated was filtrated and rinsed with water until having a neutral pH of the filtrate (ca. 150 mL). The cake was dried in vacuo over P₂O₅. The crude was purified by flash chromatography (CyHex 100% to CyHex/EtOAc 90:10) to afford a white solid (363 mg, 67%).

¹H-NMR (DMSO-d₆, 400 MHz) δ: 4.25 (s, 2H, N—CH₂); 8.12 (s, 1H, Ar); 9.95 (bs, 1H, NH).

### Intermediate 130: N-(2-amino-3,4,5-triiodobenzyl)-2,2,2-trifluoroacetamide

To a suspension of intermediate 129 (358 mg, 1.0 equiv) in EtOH (5 mL) and water (1.75 mL) was added ammonium chloride (214 mg, 7.0 equiv) and iron (224 mg, 7.0 equiv). The reaction was stirred at 25° C. for 2 h, then the suspension was diluted with EtOH (40 mL) sonicated, filtrated over Celite®, rinsed with EtOH and evaporated to dryness. The crude was purified by flash chromatography (CyHex 100% to CyHex/EtOAc 9:1) to afford a beige solid (289 mg, 85%). M/Z (M+H)⁺: 596.8.

### Intermediate 131: 6-(aminomethyl)-2,3,4-triiodoaniline

To a solution of intermediate 130 (285 mg, 1.0 equiv) in MeOH (10 mL) was added 1 N aqueous sodium hydroxide (2.39 mL, 5.0 equiv). The reaction was stirred at 25° C. for 22 h. Then, water (30 mL) was added and the precipitate was filtrated, rinsed and triturated in water (3×5 mL) to obtain a yellow solid (213 mg, 89%). M/Z (M+H)⁺: 500.7.

### Intermediate 132: 6-chloro-3,4-dihydroquinazoline-2(1H)-thione

Intermediate 132 was isolated as a white solid (211 mg, 94%) according to general procedure 2b, starting from intermediate 131 (208 mg) after trituration of the crude in DCM (3×2 mL), then in MeOH (4 mL) at 65° C. for 2 h, then in MeOH (2×2 mL) and in diethyl ether (2 mL). M/Z (M+H)⁺: 542.7.

### Intermediate 133: tert-butyl (2-amino-3-chlorobenzyl)carbamate

Intermediate 133 was isolated as an orange oil (532 mg) according to general procedure 4c, starting from 2-amino-3-chlorobenzonitrile (500 mg) after purification by flash chromatography (CyHex 100% to CyHex/EtOAc 85:15). M/Z (M[³⁵Cl]-^{t}Bu+H)⁺: 201.0.

### Intermediate 134: 2-(aminomethyl)-6-chloroaniline

Intermediate 134 was isolated as an orange oil (259 mg, 50% over 2 steps) according to general procedure 4f, starting from intermediate 133 (532 mg).

¹H-NMR (DMSO-d₆, 300 MHz) δ: 1.97 (bs, 2H, NH₂); 3.69 (s, 2H, N—CH₂); 5.42 (bs, 2H, NH₂); 6.53 (t, J7.7 Hz, 1H, Ar); 7.00-7.02 (m, 1H, Ar); 7.11 (dd, J7.8, 1.5 Hz, 1H, Ar).

### Intermediate 135: 8-chloro-3,4-dihydroquinazoline-2(1H)-thione

Intermediate 135 was isolated as a white solid (242 mg, 73%) according to general procedure 2b, starting from intermediate 134 (259 mg). M/Z (M[³⁵Cl]+H)⁺: 199.0.

### Intermediate 136: 5,5-dimethyltetrahydropyrimidine-2(1H)-thione

At 0° C., to a solution of 2,2-dimethylpropane-1,3-diamine (1.00 g, 1.0 equiv) in DCM (16 mL) was added a solution of di(1H-imidazol-1-yl)methanethione (1.74 g, 1.0 equiv) in DCM (32 mL). The reaction mixture was stirred at 0° C. for 4 h and then concentrated to dryness. The residue was triturated in MeCN (10 mL) at 25° C. for 2 h, then the solid was filtered and washed with MeCN to obtain a white solid (500 mg, 35%). M/Z (M+H)⁺: 145.1.

### Intermediate 137: 1′H-spiro[cyclopropane-1,4′-quinazoline]-2′(3′H)-thione

Intermediate 137 was isolated as a yellow solid (490 mg, 76%) according to general procedure 2b, starting from 2-(1-aminocyclopropyl)aniline (500 mg). M/Z (M+H)⁺: 191.0.

### Intermediate 138: octahydroquinazoline-2(1H)-thione

At 0° C., to a solution of 2-(aminomethyl)cyclohexan-1-amine (500 mg, 1.0 equiv) in DCM (7 mL) was added a solution of di(1H-imidazol-1-yl)methanethione (695 mg, 1.0 equiv) in DCM (14 mL). The reaction mixture was stirred at 0° C. for 4 h, then it was concentrated in vacuo. The residue was triturated in MeCN (10 mL) at 25° C. for 1 h. The solid was filtrated and washed with MeCN. The filtrate was concentrated to dryness, purified by flash chromatography (20 μm, DCM 100% to DCM/MeOH 90:10) and combined with the solid to obtain a white solid (380 mg, 57%). M/Z (M+H)⁺: 171.1.

### Intermediate 139: 4-phenylimidazolidine-2-thione

Intermediate 139 was isolated as a white solid (441 mg, 67%) according to general procedure 2b, starting from 1-phenylethane-1,2-diamine (500 mg). M/Z (M+H)⁺: 179.1.

### Intermediates: Electrophiles

### Intermediate 59: 3-(chloromethyl)-6,6-dimethyl-5,6-dihydroimidazo[2,1-b]thiazole hydrochloride

To a solution of 4,4-dimethylimidazolidine-2-thione (40.0 g, 1.0 equiv) in MeCN (600 mL) was added 1,3-dichloropropan-2-one (39.0 g, 1.0 equiv). The reaction mixture was stirred at 80° C. for 17 h and concentrated to dryness. The crude residue was triturated in ethylene glycol dimethyl ether at 100° C. for 2 h to afford a grey solid (26.5 g, 35%). M/Z (M³⁵[Cl]+H)⁺: 203.1.

### Intermediate 60: 3-(chloromethyl)-5,6-dihydroimidazo[2,1-b]thiazole hydrochloride

Intermediate 60 was isolated as a white solid (4.1 g, 99%) according to general procedure 1a, starting from 4,4-dimethylimidazolidine-2-thione (2.0 g, 1.0 equiv) and 1,3-dichloropropan-2-one (3.7 g, 1.5 equiv). M/Z (M[³⁵Cl]+H)⁺=174.5

### Intermediate 61: trans-3-(chloromethyl)-4a,5,6,7,8,8a-hexahydrobenzo[4,5]imidazo[2,1-b]thiazole hydrochloride

Intermediate 61 was isolated as a white solid (45 mg, 45%) according to general procedure 1a, starting from intermediate 1 (60 mg, 1.0 equiv) and 1,3-dichloropropan-2-one (49 mg, 1.0 equiv). M/Z (M[³⁵Cl]+H)⁺=229.1.

### Intermediate 62: 6-benzyl-3-(chloromethyl)-5,6-dihydroimidazo[2,1-b]thiazole hydrochloride

Intermediate 62 was isolated as a white solid (268 mg, 86%) according to general procedure 1b, starting from intermediate 3 (200 mg, 1.0 equiv) and 1,3-dichloropropan-2-one (132 mg, 1.0 equiv). M/Z (M[³⁵Cl]+H)⁺=265.1.

Intermediate 63: 7-chloro-3-(chloromethyl)-5H-thiazolo[2,3-b]quinazoline hydrochloride Intermediate 63 was isolated as a white solid (230 mg, 74%) according to general procedure 1e, starting from intermediate 4 (200 mg, 1.0 equiv) and 1,3-dichloropropan-2-one (128 mg, 1.0 equiv) after trituration in cold MeCN. M/Z (M³⁵[Cl]+H)⁺: 271.0.

### Intermediate 64: 3-(chloromethyl)-5H-thiazolo[2,3-b]quinazoline hydrochloride

Intermediate 64 was isolated as an off-white solid (230 mg, 69%) according to general procedure 1d, starting from intermediate 5 (200 mg, 1.0 equiv) and 1,3-dichloropropan-2-one (155 mg, 1.0 equiv) after trituration in cold MeCN. M/Z (M³⁵[Cl]+H)⁺: 237.1.

### Intermediate 65: 3-(chloromethyl)-5,10-dihydrobenzo[e]thiazolo[3,2-a][1,3]diazepine hydrochloride

Intermediate 65 was isolated as a beige solid (240 mg, 75%) according to general procedure 1d, starting from intermediate 7 (200 mg, 1.0 equiv) and 1,3-dichloropropan-2-one (214 mg, 1.5 equiv) after trituration in cold MeCN. M/Z (M³⁵[Cl]+H)⁺: 251.1.

Intermediate 66: 8-chloro-3-(chloromethyl)-5H-thiazolo[2,3-b]quinazoline hydrochloride Intermediate 66 was isolated as a beige solid (200 mg, 65%) according to general procedure 1e, starting from intermediate 6 (200 mg, 1.0 equiv) and 1,3-dichloropropan-2-one (192 mg, 1.5 equiv) after trituration in cold MeCN (2 mL). M/Z (M³⁵[Cl]+H)⁺: 270.9.

### Intermediate 67: 3-(chloromethyl)-6-(4-chlorophenyl)-5,6-dihydroimidazo[2,1-b]thiazole hydrochloride

Intermediate 67 was isolated as a beige solid (210 mg, 69%) according to general procedure 1a, starting from intermediate 8 (200 mg, 1.0 equiv) and 1,3-dichloropropan-2-one (179 mg, 1.5 equiv). M/Z (M[³⁵Cl]+H)⁺: 285.0.

### Intermediate 68: 3-(chloromethyl)-6-cyclohexyl-5,6-dihydroimidazo[2,1-b]thiazole hydrochloride

Intermediate 68 was isolated as a beige solid (195 mg, 61%) according to general procedure 1a, starting from intermediate 9 (200 mg, 1.0 equiv) and 1,3-dichloropropan-2-one (179 mg, 1.5 equiv). M/Z (M[³⁵Cl]+H)⁺: 257.1.

### Intermediate 69: trans-3-(chloromethyl)-5,6-diphenyl-5,6-dihydroimidazo[2,1-b]thiazole hydrochloride

Intermediate 69 was isolated as a white solid (188 mg, 66%) according to general procedure 1c, starting from intermediate 10 (200 mg, 1.0 equiv) and 1,3-dichloropropan-2-one (150 mg, 1.5 equiv) after purification by preparative HPLC (column A, H₂O+0.1% HCOOH/MeCN+0.1% HCOOH 90:10 to 50:50) and freeze-drying with 1 N aqueous HCl. M/Z (M[³⁵Cl]+H)⁺: 327.1.

### Intermediate 70: 3-(chloromethyl)-6-fluoro-5H-thiazolo[2,3-b]quinazoline hydrochloride

Intermediate 70 was isolated as a white solid (250 mg, 78%) according to general procedure 1f, starting from intermediate 11 (200 mg, 1.0 equiv) and 1,3-dichloropropan-2-one (209 mg, 1.5 equiv). M/Z (M[³⁵Cl]+H)⁺: 255.0.

### Intermediate 71: 3-(chloromethyl)-5H-pyrido[2,3-d]thiazolo[3,2-a]pyrimidine dihydrochloride

To a suspension of 15 (50 mg, 1.0 equiv) in MeCN (1.5 mL) was added 1,3-dichloropropan-2-one (192 mg, 5.0 equiv). The reaction was heated at 50° C. for 55 h and then at 80° C. for 72 h. The resulting solid was filtered and washed with MeCN and triturated in EtOH (2×2 mL) to obtain a pale yellow solid (50 mg, 53%). M/Z (M[³⁵Cl]+H)⁺: 238.0

### Intermediate 72: 3-(chloromethyl)-5,6-dihydrobenzo[d]thiazolo[3,2-a][1,3]diazepine dhydrochloride

Intermediate 72 was isolated as a white solid (260 mg, 81%) according to general procedure 1b, starting from intermediate 13 (200 mg, 1.0 equiv) and 1,3-dichloropropan-2-one (214 mg, 1.5 equiv). M/Z (M[³⁵Cl]+H)⁺: 250.9.

### Intermediate 73: 3-(chloromethyl)-6-methyl-6-phenyl-5,6-dihydroimidazo[2,1-b]thiazole hydrochloride

Intermediate 73 was isolated as a white solid (250 mg, 80%) according to general procedure 1a, starting from intermediate 16 (200 mg, 1.0 equiv) and 1,3-dichloropropan-2-one (198 mg, 1.5 equiv) after trituration in Et₂O (15 mL). M/Z (M[³⁵Cl]+H)⁺: 265.0.

### Intermediate 74: 3-(chloromethyl)-5-phenyl-5H-thiazolo[2,3-b]quinazoline hydrochloride

Intermediate 74 was isolated as a white solid (200 mg, 69%) according to general procedure 1b, starting from intermediate 14 (200 mg, 1.0 equiv) and 1,3-dichloropropan-2-one (158 mg, 1.5 equiv). M/Z (M[³⁵Cl]+H)⁺: 313.0.

### Intermediate 75: 3-(chloromethyl)-5,5-dimethyl-2,3-dihydro-5H-thiazolo[2,3-b]quinazolin-3-ol hydrochloride

Intermediate 75 was isolated as a beige solid (170 mg, 51%) according to general procedure 1c, starting from intermediate 12 (200 mg, 1.0 equiv) and 1,3-dichloropropan-2-one (198 mg, 1.5 equiv) after purification by preparative HPLC (column A, H₂O+0.1% HCOOH/MeCN+0.1% HCOOH 95:5 to 55:45) and freeze-drying with 1 N aqueous HCl (2 equiv). M/Z (M[³⁵Cl]+H)⁺: 283.0.

### Intermediate 76: 3-(chloromethyl)-6-(4-methoxybenzyl)-6-methyl-5,6-dihydroimidazo[2,1-b]thiazole hydrochloride

Intermediate 76 was isolated as a white solid (275 mg, 94%) according to general procedure 1c, starting from intermediate 17 (200 mg, 1.0 equiv) and 1,3-dichloropropan-2-one (198 mg, 1.5 equiv) after trituration in Et₂O (15 mL). M/Z (M[³⁵Cl]+H)⁺: 309.0.

### Intermediate 77: 3-(chloromethyl)-6,7-dimethoxy-2,3-dihydrobenzo[4,5]imidazo[2,1-b]thiazol-3-ol hydrochloride

Intermediate 77 was isolated as a green solid (320 mg, quant.) according to general procedure 1g, starting from 5,6-dimethoxy-1,3-dihydro-2H-benzo[d]imidazole-2-thione (200 mg, 1.0 equiv) and 1,3-dichloropropan-2-one (604 mg, 5.0 equiv). M/Z (M[³⁵Cl]+H)⁺: 301.0.

### Intermediate 78: 3-(chloromethyl)-6-(thiophen-2-ylmethyl)-5,6-dihydroimidazo[2,1-b]thiazole hydrochloride

Intermediate 78 was isolated as a beige solid (88 mg, 80%) according to general procedure 1a, starting from intermediate 24 (71 mg, 1.0 equiv) and 1,3-dichloropropan-2-one (68 mg, 1.5 equiv). M/Z (M[³⁵Cl]+H)⁺: 270.9.

### Intermediate 79: 7-bromo-3-(chloromethyl)-5H-thiazolo[2,3-b]quinazoline hydrochloride

Intermediate 79 was isolated as a beige solid (190 mg, 66%) according to general procedure 1d, starting from intermediate 26 (200 mg, 1.0 equiv) and 1,3-dichloropropan-2-one (157 mg, 1.5 equiv). M/Z (M[³⁵Cl][⁸¹Br]+H)⁺: 316.8.

### Intermediate 80: 8-bromo-3-(chloromethyl)-5H-thiazolo[2,3-b]quinazoline hydrochloride

Intermediate 80 was isolated as a beige solid (135 mg, 47%) according to general procedure 1f, starting from intermediate 25 (200 mg, 1.0 equiv) and 1,3-dichloropropan-2-one (157 mg, 1.5 equiv). M/Z (M[³⁵Cl][⁸¹Br]+H)⁺: 316.7.

### Intermediate 81: 3-(chloromethyl)-2,3-dihydrobenzo[4,5]imidazo[2,1-b]thiazol-3-ol hydrochloride

A suspension of 1,3-dihydro-2H-benzo[d]imidazole-2-thione (200 mg, 1.0 equiv) and 1,3-dichloropropan-2-one (845 mg, 5.0 equiv) in MeCN (6 mL) was stirred at 25° C. for 5 h. The resulting solid was filtered and washed with MeCN to obtain a white solid (340 mg, 92%). M/Z (M[³⁵Cl]+H)⁺: 240.9.

### Intermediate 82: 2-(2-((tert-butyldimethylsilyl)oxy)ethyl)isoindoline

Intermediate 82 was isolated as a brown liquid (1.43 g, 62%) according to general procedure 5a, starting from isoindoline (1.00 g) and (2-bromoethoxy)(tert-butyl)dimethylsilane after purification by flash chromatography (DCM 100% to DCM/MeOH 90:10).

¹H-NMR (DMSO-d₆, 400 MHz) δ: 0.06 (s, 6H, 2 Si—CH₃); 0.89 (s, 9H, (CH₃)₃); 2.80 (t, J6.3 Hz, 2H, O—CH₂—CH₂—N); 3.75 (t, J6.3 Hz, 2H, O—CH₂—CH₂—N); 3.89 (s, 4H, 2 N—CH₂—Ar); 7.16-7.23 (m, 4H, 4 Ar).

### Intermediate 83: 2-(isoindolin-2-yl)ethan-1-ol hydrochloride

Intermediate 83 was isolated as a black solid (430 mg, 99%) according to general procedure 5b, starting from intermediate 82 (602 mg).

¹H-NMR (DMSO-d₆, 400 MHz) δ: 3.45-3.49 (m, 2H, O-CH₂—CH₂—N); 3.79-3.82 (m, 2H, O—CH₂—CH₂—N); 4.54-4.57 (m, 2H, N—CH₂—Ar); 4.77-4.80 (m, 2H, N—CH₂—Ar); 5.37 (bs, 1H, OH); 7.36-7.42 (m, 4H, 4 Ar); 11.15 (bs, 1H, HCl salt).

### Intermediate 84: 2-(2-chloroethyl)isoindoline hydrochloride

Intermediate 84 was isolated as a silver solid (1010 mg, 96%) according to general procedure 5c, starting from intermediate 83 (959 mg).

¹H-NMR (DMSO-d₆, 400 MHz) δ: 3.81 (t, J6.3 Hz, 2H, Cl—CH₂—CH₂—N); 4.07 (t, J6.3 Hz, 2H, Cl—CH₂—CH₂—N); 4.53-4.66 (m, 2H, N—CH₂—Ar); 4.73-4.88 (m, 2H, N—CH₂—Ar); 7.36-7.45 (m, 4H, 4 Ar); 11.61 (bs, 1H, HCl salt).

### Intermediate 85: 1-methylpyrrolidin-3-yl methanesulfonate

Intermediate 85 was isolated as an orange oil (157 mg, 89%) according to general procedure 5e, starting from1-methylpyrrolidin-3-ol (100 mg). M/Z (M+H)⁺: 179.6.

### Intermediate 86: 7-chloro-3-(chloromethyl)-5H-thiazolo[2,3-b]quinazoline

Intermediate 63 was suspended in saturated aqueous NaHCO₃ (75 mL). Then it was extracted with DCM (2×30 mL), washed with brine, filtered through a hydrophobic cartridge and concentrated to dryness to obtain a beige solid (219 mg, 99%). M/Z (M+H)⁺: 271.0.

### Intermediate 87: 2-bromo-7-chloro-3-(chloromethyl)-5H-thiazolo[2,3-b]quinazoline

To a cloudy mixture of intermediate 86 (60 mg, 1.0 equiv) in DCM (1.5 mL) was added in one portion N-bromosuccinimide (40 mg, 1.0 equiv) and the reaction mixture was stirred at 25° C. for 1.5 h. The resulting precipitate was isolated by centrifugation, triturated in DCM (3×2 mL), and in diethyl ether (2 mL) to obtain a white solid (31 mg). M/Z (M³⁵[Cl]₂⁷⁹[Br]+H)⁺: 348.9.

### Intermediate 88: 7-chloro-3-(chloromethyl)-2,3-dihydro-5H-thiazolo[2,3-b]quinazolin-3-ol hydrochloride

Intermediate 88 was isolated as a beige solid (604 mg, 89%) according to general procedure 1a, starting from intermediate 4 (414 mg, 1.0 equiv) and 1,3-dichloropropan-2-one (265 mg, 1.0 equiv). M/Z (M[³⁵Cl]₂+H)⁺: 288.8.

### Intermediate 89: 6-chloro-3-(chloromethyl)-2,3-dihydro-5H-thiazolo[2,3-b]quinazolin-3-ol hydrochloride

Intermediate 89 was isolated as a white solid (150 mg, 86%) according to general procedure 1g, starting from intermediate 30 (106 mg) and 1,3-dichloropropan-2-one (68 mg, 1.0 equiv). M/Z (M[³⁵Cl]₂+H)⁺: 289.0.

### Intermediate 90: 3-(chloromethyl)-8-fluoro-2,3-dihydro-5H-thiazolo[2,3-b]quinazolin-3-ol hydrochloride

Intermediate 90 was isolated as a beige solid (172 mg, 84%) according to general procedure 1g, starting from intermediate 34 (106 mg) and 1,3-dichloropropan-2-one (84 mg, 1.0 equiv). M/Z (M[³⁵Cl]+H)⁺: 273.0.

### Intermediate 91: 3-(chloromethyl)-7-fluoro-2,3-dihydro-5H-thiazolo[2,3-b]quinazolin-3-ol hydrochloride

Intermediate 91 was isolated as a beige solid (187 mg, 83%) according to general procedure 1g, starting from intermediate 39 (133 mg) and 1,3-dichloropropan-2-one (93 mg, 1.0 equiv). M/Z (M[³⁵Cl]+H)⁺: 273.0.

### Intermediate 92: 9-bromo-3-(chloromethyl)-2,3-dihydro-5H-thiazolo[2,3-b]quinazolin-3-ol hydrochloride

Intermediate 92 was isolated as a white solid (120 mg, 66%) according to general procedure 1g, starting from intermediate 42 (120 mg) and 1,3-dichloropropan-2-one (69 mg, 1.1 equiv). M/Z (M[³⁵Cl][⁷⁹Br]+H)⁺: 333.0

### Intermediate 93: 7-chloro-3-(chloromethyl)-9-fluoro-2,3-dihydro-5H-thiazolo[2,3-b]quinazolin-3-ol hydrochloride

Intermediate 93 was isolated as a white solid (148 mg, 93%) according to general procedure 1g, starting from intermediate 46 (100 mg) and 1,3-dichloropropan-2-one (65 mg, 1.1 equiv). M/Z (M[³⁵Cl]₂+H)⁺: 306.9.

### Intermediate 94: 6-(4-chlorobenzyl)-3-(chloromethyl)-5,6-dihydroimidazo[2,1-b]thiazole hydrochloride

Intermediate 94 was isolated as a white solid (40 mg, 39%) according to general procedure 1a, starting from intermediate 58 (70 mg) and 1,3-dichloropropan-2-one (59 mg, 1.5 equiv). M/Z (M[³⁵Cl]₂+H)⁺: 299.0.

### Intermediate 140: (S)-6-((1H-indol-3-yl)methyl)-3-(chloromethyl)-5,6-dihydroimidazo[2,1-b]thiazole hydrochloride

Intermediate 140 was isolated as a brown solid (31 mg) according to general procedure 1a, starting from intermediate 97 (43 mg, 0.16 mmol) and 1,3-dichloropropan-2-one (35 mg, 1.7 equiv). M/Z (M[³⁵Cl]₂+H)⁺: 304.1.

### Intermediate 141: 6-benzyl-3-(chloromethyl)-2-iodo-5,6-dihydroimidazo[2,1-b]thiazole hydrochloride

In a MW vial (2-5 mL), to a suspension of intermediate 62 (70 mg, 1.0 equiv) in MeCN (1.5 mL) was added iodine (88 mg, 1.5 equiv) and silver sulfate (110 mg, 1.5 equiv). The reaction was stirred at 25° C. in the dark for 2 h. The reaction mixture then was filtered and evaporated to dryness. The crude was purified by preparative HPLC (column B, H₂O+0.1% HCOOH/MeCN+0.1% HCOOH 95:5 to 55:45) and freeze-dried with 1 N aqueous HCl (5 equiv) to obtain a white solid (57 mg, 57%). M/Z (M[³⁵Cl]+H)⁺: 391.0.

### Intermediate 142: (S)-6-(3-chlorobenzyl)-3-(chloromethyl)-5,6-dihydroimidazo[2,1-b]thiazole hydrochloride

Intermediate 142 was isolated as a white solid (78 mg, 57%) according to general procedure 1h, starting from intermediate 100 (93 mg) and 1,3-dichloropropan-2-one (56 mg, 1.1 equiv). M/Z (M[³⁵Cl]₂+H)⁺: 299.0.

### Intermediate 143: 3-(chloromethyl)-6-(3-methylbenzyl)-5,6-dihydroimidazo[2,1-b]thiazole hydrochloride

Intermediate 143 was isolated as a white solid (78 mg, 43%) according to general procedure 1h, starting from intermediate 103 (120 mg) and 1,3-dichloropropan-2-one (80 mg, 1.1 equiv). M/Z (M[³⁵Cl]+H)⁺: 279.0.

### Intermediate 144: 1-(2-((tert-butyldimethylsilyl)oxy)ethyl)indoline

To a solution of indoline (329 μL, 2.94 mmol, 1.0 equiv) in MeCN (14 mL) was added (2-bromoethoxy)(tert-butyl)dimethylsilane (1486 mg, 2.1 equiv) and potassium carbonate (886 mg, 2.1 equiv). The reaction mixture was stirred at 25° C. for 5 d and heated at 80° C. for 3 d. The reaction mixture was allowed to cool down to rt, then filtered. The filtrate was concentrated to dryness and purified by flash chromatography (CyHex 100% to CyHex/EtOAc 70:30) to obtain intermediate 144 (842 mg) as a light-orange oil. M/Z (M+H)⁺: 278.0.

### Intermediate 145: 2-(indolin-1-yl)ethan-1-ol hydrochloride

Intermediate 145 was isolated as a red oil (509 mg, 87% over 2 steps) according to general procedure 5b, starting from intermediate 144 (2.94 mmol). M/Z (M+H)⁺: 163.9.

### Intermediate 146: 1-(2-iodoethyl)indoline

Intermediate 146 was isolated as an orange oil (135 mg) according to general procedure 5d, starting from intermediate 145 (509 mg) after purification by flash chromatography (CyHex 100% to CyHex/EtOAc 70:30). M/Z (M+H)⁺: 274.0.

### Intermediate 147: 3-(chloromethyl)-6-phenyl-5,6-dihydroimidazo[2,1-b]thiazole hydrochloride

Intermediate 147 was isolated as a beige solid (100 mg, 31%) according to general procedure 1a, starting from intermediate 139 (200 mg, 1.0 equiv) and 1,3-dichloropropan-2-one (214 mg, 1.5 equiv) after recrystallisation in MeCN (12 mL). M/Z (M[³⁵Cl]+H)⁺: 251.1

### Intermediate 148: 3-(chloromethyl)-6-(3-fluorobenzyl)-5,6-dihydroimidazo[2,1-b]thiazole hydrochloride

Intermediate 148 was isolated as a white solid (93 mg, 70%) according to general procedure 1a, starting from intermediate 111 (87 mg, 1.0 equiv) and 1,3-dichloropropan-2-one (58 mg, 1.1 equiv). M/Z (M[³⁵Cl]+H)⁺: 283.0.

### Intermediate 149: 3-(chloromethyl)-6-(4-methylbenzyl)-5,6-dihydroimidazo[2,1-b]thiazole hydrochloride

Intermediate 149 was isolated as a white solid (141 mg, 80%) according to general procedure 1a, starting from intermediate 114 (116 mg, 1.0 equiv) and 1,3-dichloropropan-2-one (79 mg, 1.1 equiv). M/Z (M[³⁵Cl]+H)⁺: 279.0.

### Intermediate 150: 6-(2-chlorobenzyl)-3-(chloromethyl)-5,6-dihydroimidazo[2,1-b]thiazole hydrochloride

Intermediate 150 was isolated as a white solid (65 mg, 67%) according to general procedure 1a, starting from intermediate 117 (66 mg, 1.0 equiv) and 1,3-dichloropropan-2-one (41 mg, 1.1 equiv). M/Z (M[³⁵Cl]₂+H)⁺: 299.0.

### Intermediate 151: (R)-3-(chloromethyl)-6-(4-methoxybenzyl)-5,6-dihydroimidazo[2,1-b]thiazole hydrochloride

Intermediate 151 was isolated as a white solid (86 mg, 54%) according to general procedure 1h, starting from intermediate 120 (106 mg, 1.0 equiv) and 1,3-dichloropropan-2-one (67 mg, 1.1 equiv). M/Z (M[³⁵Cl]+H)⁺: 295.0.

### Intermediate 152: 1-(2-chloroethyl)-3,3-difluoropyrrolidine hydrochloride

Intermediate 152 was isolated as a yellow oil (378 mg) according to general procedure 5c, starting from 2-(3,3-difluoropyrrolidin-1-yl)ethan-1-ol (200 mg, 1.32 mmol). M/Z (M[³⁵Cl]+H)⁺: 169.9.

### Intermediate 153: 3-(chloromethyl)-6-phenethyl-5,6-dihydroimidazo[2,1-b]thiazole hydrochloride

Intermediate 153 was isolated as a white solid (74 mg, 44% over 2 steps) according to general procedure 1h, starting from intermediate 123 (122 mg, 1.0 equiv) and 1,3-dichloropropan-2-one (74 mg, 1.1 equiv) after purification by preparative HPLC (column B, H₂O+0.1% HCOOH/MeCN+0.1% HCOOH 95:5 to 55:45) and freeze-drying with 1 N aqueous HCl (5.0 equiv). M/Z (M[³⁵Cl]+H)⁺: 279.0.

### Intermediate 154: 1-(2-((tert-butyldimethylsilyl)oxy)ethyl)-3-methoxypyrrolidine

Intermediate 154 was isolated as an orange oil (215 mg, 57%) according to general procedure 5a, starting from 3-methoxypyrrolidine hydrochloride (200 mg) and (2-bromoethoxy)(tert-butyl)dimethylsilane after by flash chromatography (DCM 100% to DCM/MeOH 90:10). M/Z (M+H)⁺: 260.2.

### Intermediate 155: 2-(3-methoxypyrrolidin-1-yl)ethan-1-ol hydrochloride

Intermediate 155 was isolated as a black solid (228 mg) according to general procedure 5b, starting from intermediate 154 (215 mg, 0.83 mmol). M/Z (M+H)⁺: 145.9.

### Intermediate 156: 1-(2-chloroethyl)-3-methoxypyrrolidine hydrochloride

Intermediate 156 was isolated as a brown oil (108 mg) according to general procedure 5c, starting from intermediate 155 (0.83 mmol). M/Z (M[³⁵Cl]+H)⁺: 163.9.

### Intermediate 157: 1-(2-((tert-butyldimethylsilyl)oxy)ethyl)-2-phenylpyrrolidine

Intermediate 157 was isolated as a yellow oil (182 mg, 44%) according to general procedure 5a, starting from 2-phenylpyrrolidine (200 mg) and (2-bromoethoxy)(tert-butyl)dimethylsilane after purification by flash chromatography (DCM 100% to DCM/MeOH 80:20). M/Z (M+H)⁺: 306.0.

### Intermediate 158: 2-(2-phenylpyrrolidin-1-yl)ethan-1-ol hydrochloride

Intermediate 158 was isolated as a purple solid (148 mg) according to general procedure 5b, starting from intermediate 157 (182 mg, 0.60 mmol). M/Z (M+H)⁺: 192.0.

### Intermediate 159: 1-(2-chloroethyl)-2-phenylpyrrolidine hydrochloride

Intermediate 159 was isolated as a brown oil (340 mg) according to general procedure 5c, starting from intermediate 158 (0.60 mmol). M/Z (M[³⁵Cl]+H)⁺: 209.9.

### Intermediate 160: 1-(1-chloropropan-2-yl)pyrrolidine hydrochloride

Intermediate 160 was isolated as a beige oil (235 mg) according to general procedure 5c, starting from 1-(1-chloropropan-2-yl)pyrrolidine (200 mg). M/Z (M+H)⁺: 147.9.

### Intermediate 161: 1-(2-((tert-butyldimethylsilyl)oxy)ethyl)-2-methylpyrrolidine

To a solution of 2-methylpyrrolidine (120 μL, 1.17 mmol, 1.0 equiv) in THE (5 mL) was added potassium carbonate (179 mg, 1.1 equiv) and (2-bromoethoxy)(tert-butyl)dimethylsilane (277 μL, 1.1 equiv). The reaction was stirred at rt for 18 h and was filtered. The solid was washed with THE (10 mL) and the filtrate was partially reduced in vacuo to get a yellow solution that was used as such in the next step. M/Z (M+H)⁺: 244.2.

### Intermediate 162: 2-(2-methylpyrrolidin-1-yl)ethan-1-ol hydrochloride

Intermediate 162 was isolated as a brown oil according to general procedure 5b, starting from intermediate 161 (1.17 mmol). M/Z (M+H)⁺: 129.9.

### Intermediate 163: 1-(2-chloroethyl)-2-methylpyrrolidine hydrochloride

Intermediate 163 was isolated as a brown oil (158 mg) according to general procedure 5c, starting from intermediate 162 (1.17 mmol). M/Z (M[³⁵Cl]+H)⁺: 147.8.

### Intermediate 164: 3-(chloromethyl)-6-(4-fluorobenzyl)-5,6-dihydroimidazo[2,1-b]thiazole hydrochloride

Intermediate 164 was isolated as a white solid (145 mg, 70%) according to general procedure 1a, starting from intermediate 126 (136 mg, 1.0 equiv) and 1,3-dichloropropan-2-one (90 mg, 1.1 equiv). M/Z (M[³⁵Cl]+H)⁺: 282.9.

### Intermediate 165: 5-(2-((tert-butyldimethylsilyl)oxy)ethyl)-1,1-difluoro-5-azaspiro[2.4]heptane

To a solution of 1,1-difluoro-5-azaspiro[2.4]heptane hydrochloride (300 mg, 1.77 mmol, 1.0 equiv) in THE (6 mL) was added potassium carbonate (538 mg, 2.2 equiv) and (2-bromoethoxy)(tert-butyl)dimethylsilane (417 μL, 1.1 equiv).

The reaction was stirred at rt for 18 h and was filtered. The solid was washed with THE (5 mL) and the filtrate was partially reduced in vacuo to get a yellow solution that was used as such in the next step. M/Z (M+H)⁺: 292.1.

### Intermediate 166: 2-(1,1-difluoro-5-azaspiro[2.4]heptan-5-yl)ethan-1-ol hydrochloride

Intermediate 166 was isolated as a colorless oil according to general procedure 5b, starting from intermediate 165 (1.77 mmol). M/Z (M+H)⁺: 177.9.

### Intermediate 167: 5-(2-chloroethyl)-1,1-difluoro-5-azaspiro[2.4]heptane hydrochloride

Intermediate 167 was isolated as a brown oil (469 mg) according to general procedure 5c, starting from intermediate 166 (1.77 mmol). M/Z (M[³⁵Cl]+H)⁺: 195.9.

### Intermediate 168: 8-(2-((tert-butyldimethylsilyl)oxy)ethyl)-8-azabicyclo[3.2.1]octane

To a solution of 8-azabicyclo[3.2.1]octane hydrochloride (300 mg, 2.03 mmol, 1.0 equiv) in THE (7 mL) was added potassium carbonate (618 mg, 2.2 equiv) and (2-bromoethoxy)(tert-butyl)dimethylsilane (480 μL, 1.1 equiv). The reaction was stirred at rt for 18 h and was filtered. The solid was washed with THE (5 mL) and the filtrate was partially reduced in vacuo to get a yellow solution that was used as such in the next step. M/Z (M+H)⁺: 269.9.

### Intermediate 169: 2-(8-azabicyclo[3.2.1]octan-8-yl)ethan-1-ol hydrochloride

Intermediate 169 was isolated as a yellow oil according to general procedure 5b, starting from intermediate 168 (2.03 mmol). M/Z (M+H)⁺: 156.1.

### Intermediate 170: 8-(2-chloroethyl)-8-azabicyclo[3.2.1]octane hydrochloride

Intermediate 170 was isolated as an orange hygroscopic solid (530 mg) according to general procedure 5c, starting from intermediate 169 (2.03 mmol). M/Z (M[³⁵Cl]+H)⁺: 173.9.

### Intermediate 171: 1-(2-chloroethyl)-3-methylpyrrolidine hydrochloride

Intermediate 171 was isolated as a yellow oil (313 mg) according to general procedure 5c, starting from 2-(3-methylpyrrolidin-1-yl)ethan-1-ol (150 mg, 1.16 mmol). M/Z (M[³⁵Cl]+H)⁺: 147.8.

### Intermediate 172: (1S,4S)-5-(2-((tert-butyldimethylsilyl)oxy)ethyl)-2-oxa-5-azabicyclo[2.2.1]heptane

Intermediate 172 was isolated as a yellow oil (148 mg, 32%) according to general procedure 5a, starting from (1 S,4S)-2-oxa-5-azabicyclo[2.2.1]heptane hydrochloride (250 mg) and (2-bromoethoxy)(tert-butyl)dimethylsilane after purification by flash chromatography (DCM 100% to DCM/MeOH 95:5). M/Z (M+H)⁺: 258.2.

### Intermediate 173: 2-((1S,4S)-2-oxa-5-azabicyclo[2.2.1]heptan-5-yl)ethan-1-ol hydrochloride

Intermediate 173 was isolated as a light-yellow oil (125 mg) according to general procedure 5b, starting from intermediate 172 (148 mg, 0.57 mmol). M/Z (M+H)⁺: 144.2.

### Intermediate 174: (1S,4S)-5-(2-chloroethyl)-2-oxa-5-azabicyclo[2.2.1]heptane hydrochloride

Intermediate 174 was isolated as a yellow solid (102 mg, 90% over 2 steps) according to general procedure 5c, starting from intermediate 173 (0.57 mmol). M/Z (M[³⁵Cl]+H)⁺: 162.1.

### Intermediate 175: (1-(2-((tert-butyldimethylsilyl)oxy)ethyl)-3-phenylpyrrolidine

Intermediate 175 was isolated as an orange oil (345 mg, 69%) according to general procedure 5a, starting from 3-phenylpyrrolidine hydrochloride (300 mg) and (2-bromoethoxy)(tert-butyl)dimethylsilane after purification by flash chromatography (DCM 100% to DCM/MeOH 95:5). M/Z (M+H)⁺: 306.3.

### Intermediate 176: 2-(3-phenylpyrrolidin-1-yl)ethan-1-ol

Intermediate 176 was isolated as a yellow oil (125 mg) according to general procedure 5b, starting from intermediate 175 (345 mg, 1.13 mmol) after purification by flash chromatography (KPNH, CyHex 100% to CyHex/EtOAc 0:100). M/Z (M+H)⁺: 192.1.

### Intermediate 177: 1-(2-chloroethyl)-3-phenylpyrrolidine hydrochloride

Intermediate 177 was isolated as a beige solid (158 mg) according to general procedure 5c, starting from intermediate 176 (1.13 mmol). M/Z (M[³⁵Cl]+H)⁺: 210.0.

### Intermediate 178: 1-((1R)-2-chlorocyclopentyl)pyrrolidine

Intermediate 178 was isolated as a coloress oil (115 mg) according to general procedure 5e, starting from (1R,2R)-2-(pyrrolidin-1-yl)cyclopentan-1-ol (100 mg, 0.64 mmol). M/Z (M[³⁵Cl]+H)⁺: 174.0.

### Intermediate 179: 2-(2-((tert-butyldimethylsilyl)oxy)ethyl)-2-azaspiro[4.4]nonane

Intermediate 179 was isolated as a yellow oil (340 mg, 75%) according to general procedure 5a, with additionnal heating of the reaction mixture at 80° C. for 18 h, starting from 2-azaspiro[4.4]nonane (200 mg) and (2-bromoethoxy)(tert-butyl)dimethylsilane after by purification by flash chromatography (DCM 100% to DCM/MeOH 95:5). M/Z (M+H)⁺: 284.3.

### Intermediate 180: 2-(2-azaspiro[4.4]nonan-2-yl)ethan-1-ol hydrochloride

Intermediate 180 was isolated as an orange oil (206 mg, 84%) according to general procedure 5b, starting from intermediate 179 (340 mg). M/Z (M+H)⁺: 170.1.

### Intermediate 181: 2-(2-chloroethyl)-2-azaspiro[4.4]nonane hydrochloride

Intermediate 181 was isolated as a yellow solid (188 mg, 84%) according to general procedure 5c, starting from intermediate 180 (206 mg). M/Z (M[³⁵Cl]+H)⁺: 188.0.

### Intermediate 182: 3-(benzyloxy)-1-(2-((tert-butyldimethylsilyl)oxy)ethyl)pyrrolidine

Intermediate 182 was isolated as an orange oil (283 mg, 60%) according to general procedure 5a, with additional heating of the reaction mixture at 80° C. for 18 h, starting from 3-(benzyloxy)pyrrolidine (250 mg) and (2-bromoethoxy)(tert-butyl)dimethylsilane, after purification by flash chromatography (DCM 100% to DCM/MeOH 96:4). M/Z (M+H)⁺: 336.3.

### Intermediate 183: 2-(3-(benzyloxy)pyrrolidin-1-yl)ethan-1-ol hydrochloride

Intermediate 183 was isolated as an orange oil (242 mg) according to general procedure 5b, starting from intermediate 182 (283 mg, 0.84 mmol). M/Z (M+H)⁺: 222.1.

### Intermediate 184: 3-(benzyloxy)-1-(2-chloroethyl)pyrrolidine hydrochloride

Intermediate 184 was isolated as a brown oil (223 mg, 96% over 2 steps) according to general procedure 5c, starting from intermediate 183 (0.84 mmol). M/Z (M[³⁵Cl]+H)⁺: 240.0.

### Intermediate 185: 1-(2-((tert-butyldimethylsilyl)oxy)ethyl)pyrrolidine-3-carbonitrile

Intermediate 185 was isolated as a colorless oil (533 mg) according to general procedure 5a, with additional heating of the reaction mixture at 80° C. for 18 h, starting from pyrrolidine-3-carbonitrile hydrochloride (250 mg, 1.89 mmol) and (2-bromoethoxy)(tert-butyl)dimethylsilane, after purification by flash chromatography (DCM 100% to DCM/MeOH 95:5). M/Z (M+H)⁺: 255.2.

### Intermediate 186: 1-(2-hydroxyethyl)pyrrolidine-3-carbonitrile

Intermediate 186 was isolated as a yellow oil (137 mg) according to general procedure 5b, starting from intermediate 185 (1.89 mmol) after purification by flash chromatography (KPNH, DCM 100% to DCM/MeOH 95:5). M/Z (M+H)⁺: 141.1.

### Intermediate 187: 1-(2-chloroethyl)pyrrolidine-3-carbonitrile hydrochloride

Intermediate 187 was isolated as an orange oil (311 mg) according to general procedure 5c, starting from intermediate 186 (1.89 mmol). M/Z (M[³⁵Cl]+H)⁺: 159.0.

### Intermediate 188: 3-(2-chloroethyl)-1-methylpyrrolidine hydrochloride

Intermediate 188 was isolated as an brown oil (331 mg) according to general procedure 5c, starting from 2-(1-methylpyrrolidin-3-yl)ethan-1-ol (200 mg, 1.50 mmol). M/Z (M[³⁵Cl]+H)⁺: 147.9.

### Intermediate 189: (1R,4R)-5-(2-((tert-butyldimethylsilyl)oxy)ethyl)-2-oxa-5-azabicyclo[2.2.1]heptane

Intermediate 189 was isolated as a colorless oil (211 mg, 45%) according to general procedure 5a, starting from (1R,4R)-2-oxa-5-azabicyclo[2.2.1]heptane hydrochloride (250 mg) and (2-bromoethoxy)(tert-butyl)dimethylsilane, after purification by flash chromatography (DCM 100% to DCM/MeOH 96:4). M/Z (M+H)⁺: 258.2.

### Intermediate 190: 2-((1R,4R)-2-oxa-5-azabicyclo[2.2.1]heptan-5-yl)ethan-1-ol hydrochloride

Intermediate 190 was isolated as light-yellow oil (174 mg) according to general procedure 5b, starting from intermediate 189 (211 mg, 0.82 mmol). M/Z (M+H)⁺: 143.8.

### Intermediate 191: (1R,4R)-5-(2-chloroethyl)-2-oxa-5-azabicyclo[2.2.1]heptane hydrochloride

Intermediate 191 was isolated as a white solid (140 mg, 86% over 2 steps) according to general procedure 5c, starting from intermediate 190 (0.82 mmol). M/Z (M[³⁵Cl]+H)⁺: 162.1.

### Intermediate 192: 1-((1R)-2-chlorocyclohexyl)pyrrolidine

Intermediate 192 was isolated as a yellow oil (104 mg) according to general procedure 5e, starting from (1R,2R)-2-(pyrrolidin-1-yl)cyclohexan-1-ol (100 mg, 0.59 mmol). M/Z (M[³⁵Cl]+H)⁺: 188.1.

### Intermediate 193: 1-phenylpyrrolidin-3-yl methanesulfonate

To a solution of 1-phenylpyrrolidin-3-ol (100 mg, 0.58 mmol, 1.0 equiv) in DCM (3 mL) was added triethylamine (89 μL, 1.1 equiv) and methanesulfonyl chloride (50 μL, 1.1 equiv). The reaction was stirred at 25° C. for 3 h, then methanesulfonyl chloride (23 μL, 0.5 equiv) and triethylamine (41 μL, 0.5 equiv) were added and the reaction mixture was stirred at 25° C. for 30 min. The reaction mixture was quenched with water (2 mL) then extracted with DCM (2×5 mL). The organic layer was dried over magnesium sulfate then concentrated to dryness to afford an orange oil (187 mg). M/Z (M+H)⁺: 242.1.

### Intermediate 194: 3-benzyl-1-(2-((tert-butyldimethylsilyl)oxy)ethyl)pyrrolidine

Intermediate 194 was isolated as a colorless oil (211 mg, 45%) according to general procedure 5a, with additional heating of the reaction mixture at 80° C. for 18 h, starting from 3-benzylpyrrolidine (250 mg) and (2-bromoethoxy)(tert-butyl)dimethylsilane, after purification by flash chromatography (DCM 100% to DCM/MeOH 50:50) to obtain a yellow residue (412 mg, 83%). M/Z (M+H)⁺: 320.3.

### Intermediate 195: 2-(3-benzylpyrrolidin-1-yl)ethan-1-ol hydrochloride

Intermediate 195 was isolated as a brown oil (365 mg) according to general procedure 5b, starting from intermediate 194 (142 mg, 1.29 mmol). M/Z (M+H)⁺: 206.2.

### Intermediate 196: 3-benzyl-1-(2-chloroethyl)pyrrolidine hydrochloride

Intermediate 196 was isolated as an orange hygroscopic solid (496 mg) according to general procedure 5c, starting from intermediate 195 (1.29 mmol). M/Z (M[³⁵Cl]+H)⁺: 224.1.

### Intermediate 197: 4-(2-chloroethyl)morpholine hydrochloride

To a solution of 2-morpholinoethan-1-ol (923 μL, 1.0 equiv) in toluene (12 mL) was added thionyl chloride (830 μL, 1.5 equiv) and the reaction mixture was heated to 120° C. for 3 h. The resulting suspension was filtrated and the solid was triturated in butanol and diethyl ether to afford a brown solid (939 mg, 67%).

¹H-NMR (DMSO-d₆, 400 MHz) δ: 3.05-3.20 (m, 2H, Cl—CH₂); 3.40-3.50 (m, 4H, 2 N—CH₂); 3.77-3.83 (m, 2H, N—CH₂); 3.91-3.97 (m, 2H, O—CH₂); 4.07-4.03 (m, 2H, O—CH₂); 1.39 (bs, 1H, HCl salt).

### Intermediate 198: (S)-1-(2-((tert-butyldimethylsilyl)oxy)ethyl)-3-fluoropyrrolidine

Intermediate 198 was isolated as a colorless oil (476 mg) according to general procedure 5a, starting from (S)-3-fluoropyrrolidine hydrochloride (250 mg, 1.99 mmol) and (2-bromoethoxy)(tert-butyl)dimethylsilane to obtain a colorless oil. M/Z (M+H)⁺: 248.2.

### Intermediate 199: (S)-2-(3-fluoropyrrolidin-1-yl)ethan-1-ol hydrochloride

Intermediate 199 was isolated as a light-orange oil (303 mg, 90% over 2 steps) according to general procedure 5b, starting from intermediate 198 (1.99 mmol). M/Z (M+H)⁺: 134.0.

### Intermediate 200: (S)-1-(2-chloroethyl)-3-fluoropyrrolidine hydrochloride

Intermediate 200 was isolated as a brown oil (271 mg) according to general procedure 5c, starting from intermediate 199 (303 mg). M/Z (M[³⁵Cl]+H)⁺: 152.1.

### Intermediate 201: (R)-1-(2-((tert-butyldimethylsilyl)oxy)ethyl)-3-fluoropyrrolidine

Intermediate 201 was isolated as a colorless oil (533 mg) according to general procedure 5a, starting from (R)-3-fluoropyrrolidine hydrochloride (250 mg, 1.99 mmol) and (2-bromoethoxy)(tert-butyl)dimethylsilane to obtain a colorless oil. M/Z (M+H)⁺: 248.2.

### Intermediate 202: (R)-2-(3-fluoropyrrolidin-1-yl)ethan-1-ol hydrochloride

Intermediate 202 was isolated as a light-orange oil (262 mg, 78% over 2 steps) according to general procedure 5b, starting from intermediate 201 (1.99 mmol). M/Z (M+H)⁺: 134.1.

### Intermediate 203: (R)-1-(2-chloroethyl)-3-fluoropyrrolidine hydrochloride

Intermediate 203 was isolated as a brown oil (233 mg) according to general procedure 5c, starting from intermediate 202 (262 mg). M/Z (M[³⁵Cl]+H)⁺: 152.1.

### Intermediate 204: (S)-1-(2,2-difluoroethyl)pyrrolidin-3-yl methanesulfonate

Intermediate 204 was isolated as a beige solid (260 mg) according to general procedure 5f, starting from (S)-1-(2,2-difluoroethyl)pyrrolidin-3-ol (100 mg, 0.63 mmol). M/Z (M+H)⁺: 230.0.

### Intermediate 205: 1-ethylpyrrolidin-3-yl methanesulfonate

Intermediate 205 was isolated as an orange oil (125 mg) according to general procedure 5e, starting from 1-ethylpyrrolidin-3-ol (75 mg). M/Z (M+H)⁺: 194.1.

### Intermediate 206: 4-(pyrrolidin-1-yl)pentan-1-ol

To a suspension of 4-(pyrrolidin-1-yl)pentanoic acid hydrochloride (200 mg, 1.0 equiv) in MeOH (2 mL) was added ammonia 7.0 M in methanol (2.75 mL, 20 equiv). The reaction was stirred at 25° C. for 30 min and evaporated to dryness. The residue was suspended in THE (10 mL) and LAH 1.0 M in THE (1.93 mL, 2.0 equiv) was added at 0° C. The reaction mixture was stirred at 25° C. for 6 h, then cooled at 0° C. and diluted with Et₂O (20 mL). 0.2 mL of water, 0.2 mL of 5 N aqueous NaOH and then 0.6 mL of water were added successively. The white precipitate was removed by filtration and the filtrate was concentrated to dryness to obtain a colorless oil (145 mg, 96%). M/Z (M+H)⁺: 158.2.

### Intermediate 207: 1-(5-chloropentan-2-yl)pyrrolidine hydrochloride

Intermediate 207 was isolated as a brown oil (255 mg, 95%) according to general procedure 5c, starting from intermediate 206 (200 mg). M/Z (M[³⁵Cl]+H)⁺: 176.1.

### Intermediate 208: (S)-1-(1-chloropropan-2-yl)pyrrolidine hydrochloride

Intermediate 208 was isolated as a beige solid (401 mg, 94%) according to general procedure 5c, starting from (S)-2-(pyrrolidin-1-yl)propan-1-ol (300 mg). M/Z (M[³⁵Cl]+H)⁺: 148.2.

### Intermediate 209: (R)-1-(1-chloropropan-2-yl)pyrrolidine hydrochloride

Intermediate 209 was isolated as a beige solid (123 mg, 86%) according to general procedure 5c, starting from (R)-2-(pyrrolidin-1-yl)propan-1-ol (100 mg), after trituration of the crude in EtOAc (20 mL), filtration and washing of the solid with EtOAc (20 mL). M/Z (M[³⁵Cl]+H)⁺: 148.1.

### Intermediate 210: 2-(3-chloropropyl)-1-methylpyrrolidine hydrochloride

Intermediate 210 was isolated as a green oil (251 mg) according to general procedure 5c, starting from 3-(1-methyl-pyrrolidin-2-yl)-propan-1-ol (150 mg, 1.05 mmol). M/Z (M[³⁵Cl]+H)⁺: 162.2.

### Intermediate 211: 1-(4-chlorobutyl)-1H-imidazole hydrochloride

Intermediate 211 was isolated as a green oil (380 mg) according to general procedure 5c, starting from 4-(1H-imidazol-1-yl)butan-1-ol (240 mg, 1.71 mmol). M/Z (M[³⁵Cl]+H)⁺: 159.0.

### Intermediate 212: 2-(methylthio)-4,5-dihydro-1H-benzo[d][1,3]diazepine hydroiodide

To a suspension of intermediate 13 in abs. EtOH (10 mL) was added methyl iodide (150 μL, 1.4 equiv), then the reaction mixture was heated at 80° C. for 2 h and evaporated to dryness to obtain a pale brown solid (539 mg, quant.). M/Z (M+H)⁺: 193.1.

### Intermediate 213: 1-(2-iodoethyl)-1,2,3,4-tetrahydroquinoline

Intermediate 213 was isolated as an orange oil (139 mg) according to general procedure 5d, starting from 2-(1,2,3,4-tetrahydroquinolin-1-yl)ethan-1-ol (250 mg) after purification by flash chromatography (CyHex 100% to CyHex/EtOAc 50:50). M/Z (M+H)⁺: 288.1.

### EXAMPLES

The following examples of the invention were prepared according to general procedure A using the reaction conditions detailed in the following table, and isolated as described hereinafter.

| | Electrophile | Thiourea | | | | | |
|---|---|---|---|---|---|---|---|
| Example | (mass, equiv) | (mass, equiv) | Additive | Solvent | T ° C. | time | |
| 1 | intermediate 59 | intermediate 4 | — | MeCN | 80 | 21 | h |
| | (100 mg, 1.0 equiv) | (83 mg, 1.0 equiv) | | | | | |
| 2 | intermediate 63 | 4,4-dimethyl-2- | — | MeCN/EtOH | 80 | 4 | d |
| | (100 mg, 1.0 equiv) | imidazolidinethione | | 4:1 | | | |
| | | (47 mg, 1.1 equiv) | | | | | |
| 3 | intermediate 59 | intermediate 5 | — | MeCN/EtOH | 80 | 4 | d |
| | (100 mg, 1.0 equiv) | (76 mg, 1.1 equiv) | | 4:1 | | | |
| 4 | intermediate 63 | imidazolidine-2-thione | — | MeCN/EtOH | 80 | 4 | d |
| | (100 mg, 1.0 equiv) | (37 mg, 1.1 equiv) | | 4:1 | | | |
| 5 | intermediate 64 | 4,4-dimethyl-2- | — | MeCN/EtOH | 80 | 4 | d |
| | (100 mg, 1.0 equiv) | imidazolidinethione | | 4:1 | | | |
| | | (52 mg, 1.1 equiv) | | | | | |
| 6 | intermediate 59 | intermediate 6 | — | MeCN | 80 | 17 | h |
| | (85 mg, 1.0 equiv) | (78 mg, 1.1 equiv) | | | | | |
| 7 | intermediate 59 | intermediate 7 | — | MeCN | 80 | 17 | h |
| | (85 mg, 1.0 equiv) | (82 mg, 1.1 equiv) | | | | | |
| 8 | intermediate 65 | 4,4-dimethyl-2- | — | MeCN/EtOH | 80 | 44 | h |
| | (100 mg, 1.0 equiv) | imidazolidinethione | | 4:1 | | | |
| | | (50 mg, 1.1 equiv) | | | | | |
| 9 | intermediate 66 | 4,4-dimethyl-2- | — | MeCN/EtOH | 80 | 17 | h |
| | (100 mg, 1.0 equiv) | imidazolidinethione | | 4:1 | | | |
| | | (47 mg, 1.1 equiv) | | | | | |
| 10 | intermediate 60 | intermediate 4 | — | MeCN | 80 | 3 | d |
| | (75 mg, 1.0 equiv) | (78 mg, 1.1 equiv) | | | | | |
| 11 | intermediate 65 | intermediate 5 | — | MeCN/EtOH | 80 | 16 | h |
| | (100 mg, 1.0 equiv) | (63 mg, 1.1 equiv) | | 3:1 | | | |
| 12 | intermediate 64 | intermediate 5 | — | MeCN | 80 | 2 | d |
| | (100 mg, 1.0 equiv) | (66 mg, 1.1 equiv) | | | | | |
| 13 | intermediate 61 | intermediate 5 | — | MeCN | 80 | 16 | h |
| | (100 mg, 1.0 equiv) | (68 mg, 1.1 equiv) | | | | | |
| 14 | intermediate 67 | intermediate 5 | — | MeCN | 80 | 45 | h |
| | (100 mg, 1.0 equiv) | (56 mg, 1.1 equiv) | | | | | |
| 15 | intermediate 68 | intermediate 5 | — | MeCN | 80 | 22 | h |
| | (100 mg, 1.0 equiv) | (62 mg, 1.1 equiv) | | | | | |
| 16 & 17 | intermediate 69 | intermediate 5 | — | MeCN | 80 | 26 | h |
| | (100 mg, 1.0 equiv) | (49 mg, 1.1 equiv) | | | | | |
| 18 | intermediate 70 | intermediate 5 | — | MeCN | 80 | 3 | d |
| | (100 mg, 1.0 equiv) | (62 mg, 1.1 equiv) | | | | | |
| 19 | intermediate 63 | intermediate 5 | — | MeCN/EtOH | 80 | 2 | d |
| | (100 mg, 1.0 equiv) | (59 mg, 1.1 equiv) | | 3:1 | | | |
| 20 | intermediate 63 | intermediate 3 | — | MeCN/EtOH | 80 | 2 | d |
| | (100 mg, 1.0 equiv) | (69 mg, 1.1 equiv) | | 3:1 | | | |
| 21 | intermediate 65 | intermediate 12 | — | MeCN/EtOH | 80 | 18 | h |
| | (100 mg, 1.0 equiv) | (74 mg, 1.1 equiv) | | 3:1 | | | |
| 22 | intermediate 65 | intermediate 8 | — | MeCN/EtOH | 80 | 24 | h |
| | (100 mg, 1.0 equiv) | (82 mg, 1.1 equiv) | | 3:1 | | | |
| 23 | intermediate 65 | intermediate 11 | — | MeCN/EtOH | 80 | 42 | h |
| | (100 mg, 1.0 equiv) | (76 mg, 1.2 equiv) | | 3:1 | | | |
| 24 | intermediate 65 | intermediate 10 | — | MeCN/EtOH | 80 | 24 | h |
| | (100 mg, 1.0 equiv) | (97 mg, 1.1 equiv) | | 3:1 | | | |
| 25 | intermediate 65 | intermediate 13 | — | MeCN/EtOH | 80 | 18 | h |
| | (100 mg, 1.0 equiv) | (68 mg, 1.1 equiv) | | 3:1 | | | |
| 26 | intermediate 65 | intermediate 9 | — | MeCN/EtOH | 80 | 24 | h |
| | (100 mg, 1.0 equiv) | (64 mg, 1.0 equiv) | | 3:1 | | | |
| 27 | intermediate 65 | intermediate 14 | — | MeCN/EtOH | 80 | 18 | h |
| | (100 mg, 1.0 equiv) | (92 mg, 1.1 equiv) | | 3:1 | | | |
| 28 | intermediate 71 | intermediate 5 | — | MeCN/EtOH | 80 | 24 | h |
| | (50 mg, 1.0 equiv) | (33 mg, 1.1 equiv) | | 3:1 | | | |
| 29 | intermediate 64 | intermediate 2 | — | MeCN/EtOH | 80 | 42 | h |
| | (100 mg, 1.0 equiv) | (64 mg, 1.1 equiv) | | 3:1 | | | |
| 30 | intermediate 65 | intermediate 16 | — | MeCN/EtOH | 80 | 18 | h |
| | (100 mg, 1.0 equiv) | (74 mg, 1.1 equiv) | | 3:1 | | | |
| 31 | intermediate 65 | intermediate 15 | — | DMF | 80 | 48 | h |
| | (50 mg, 1.0 equiv) | (32 mg, 1.1 equiv) | | | | | |
| 32 | intermediate 64 | intermediate 1 | — | MeCN/EtOH | 80 | 3 | d |
| | (100 mg, 1.0 equiv) | (63 mg, 1.1 equiv) | | 3:1 | | | |
| 33 | intermediate 64 | intermediate 3 | — | MeCN/EtOH | 80 | 40 | h |
| | (100 mg, 1.0 equiv) | (77 mg, 1.1 equiv) | | 3:1 | | | |
| 34 | intermediate 65 | intermediate 17 | — | MeCN/EtOH | 80 | 18 | h |
| | (100 mg, 1.0 equiv) | (77 mg, 1.1 equiv) | | 3:1 | | | |
| 35 | intermediate 72 | intermediate 5 | — | MeCN | 80 | 18 | h |
| | (100 mg, 1.0 equiv) | (63 mg, 1.1 equiv) | | | | | |
| 36 | intermediate 64 | 1-methylimidazolidine-2- | — | MeCN/EtOH | 80 | 5 | d |
| | (100 mg, 1.0 equiv) | thione | | 3:1 | | | |
| | | (51 mg, 1.2 equiv) | | | | | |
| 37 | intermediate 65 | intermediate 18 | — | MeCN/EtOH | 80 | 3 | d |
| | (100 mg, 1.0 equiv) | (61 mg, 1.1 equiv) | | 3:1 | | | |
| 38 | intermediate 73 | intermediate 5 | — | MeCN/EtOH | 80 | 42 | h |
| | (100 mg, 1.0 equiv) | (60 mg, 1.1 equiv) | | 3:1 | | | |
| 39 | intermediate 64 | intermediate 4 | — | MeCN/EtOH | 80 | 6 | d |
| | (100 mg, 1.0 equiv) | (87 mg, 1.1 equiv) | | 3:1 | | | |
| 40 | intermediate 63 | intermediate 1 | — | MeCN/EtOH | 80 | 32 | h |
| | (100 mg, 1.0 equiv) | (56 mg, 1.1 equiv) | | 3:1 | | | |
| 41 | intermediate 63 | intermediate 2 | — | MeCN/EtOH | 80 | 2 | d |
| | (100 mg, 1.0 equiv) | (57 mg, 1.1 equiv) | | 3:1 | | | |
| 42 | intermediate 66 | intermediate 5 | — | MeCN/EtOH | 80 | 17 | h |
| | (100 mg, 1.0 equiv) | (58 mg, 1.1 equiv) | | 3:1 | | | |
| 43 | intermediate 74 | intermediate 5 | — | MeCN/EtOH | 80 | 50 | h |
| | (100 mg, 1.0 equiv) | (52 mg, 1.1 equiv) | | 3:1 | | | |
| 44 | intermediate 63 | intermediate 4 | — | DMA | 80 | 36 | h |
| | (100 mg, 1.0 equiv) | (77 mg, 1.2 equiv) | | | | | |
| 45 | intermediate 63 | 1-methylimidazolidine-2- | — | DMA | 80 | 48 | h |
| | (100 mg, 1.0 equiv) | thione | | | | | |
| | | (189 mg, 5.0 equiv) | | | | | |
| 46 | intermediate 76 | intermediate 5 | — | MeCN | 80 | 24 | h |
| | (100 mg, 1.0 equiv) | (52 mg, 1.1 equiv) | | | | | |
| 47 | intermediate 63 | intermediate 19 | — | MeCN/EtOH | 80 | 7 | d |
| | (50 mg, 1.0 equiv) | (156 mg, 5.0 equiv) | | 3:1 | | | |
| 48 | intermediate 63 | intermediate 20 | — | MeCN/EtOH | 80 | 5 | d |
| | (50 mg, 1.0 equiv) | (117 mg, 5.0 equiv) | | 3:1 | | | |
| 49 | intermediate 63 | intermediate 21 | — | MeCN/EtOH | 80 | 5 | d |
| | (50 mg, 1.0 equiv) | (127 mg, 5.0 equiv) | | 3:1 | | | |
| 50 | 1-(2-chloroethyl)piperidine | intermediate 3 | NaI | MeCN | 80 | 18 | h |
| | hydrochloride | (64 mg, 1.0 equiv) | (100 mg, | | | | |
| | (123 mg, 2.0 equiv) | | 2.0 equiv) | | | | |
| 51 | 2-(chloromethyl)imidazo[1,2- | intermediate 3 | NaI | EtOH | 80 | 18 | h |
| | a]pyrimidine | (150 mg, 1.0 equiv) | (234 mg, | | | | |
| | (261 mg, 2.0 equiv) | | 2.0 equiv) | | | | |
| 52 | 1-(3-chloropropyl)pyrrolidine | intermediate 3 | NaI | EtOH | 80 | 18 | h |
| | hydrochloride | (100 mg, 1.0 equiv) | (156 mg, | | | | |
| | (192 mg, 2.0 equiv) | | 2.0 equiv) | | | | |
| 53 | 2-(chloromethyl)-1- | intermediate 3 | NaI | EtOH | 80 | 18 | h |
| | methylpyrrolidine | (100 mg, 1.0 equiv) | (156 mg, | | | | |
| | hydrochloride | | 2.0 equiv) | | | | |
| | (177 mg, 2.0 equiv) | | | | | | |
| 54 | 1-(2-chloroethyl)pyrrolidine | intermediate 3 | NaI | EtOH | 80 | 18 | h |
| | hydrochloride | (100 mg, 1.0 equiv) | (156 mg, | | | | |
| | (177 mg, 2.0 equiv) | | 2.0 equiv) | | | | |
| 55 | 4-(3-chloropropyl)pyridine | intermediate 3 | NaI | EtOH | 80 | 18 | h |
| | hydrochloride | (100 mg, 1.0 equiv) | (156 mg, | | | | |
| | (200 mg, 2.0 equiv) | | 2.0 equiv) | | | | |
| 56 | 4-(bromomethyl)pyridine | intermediate 3 | NaI | EtOH | 80 | 18 | h |
| | hydrobromide | (100 mg, 1.0 equiv) | (156 mg, | | | | |
| | (263 mg, 2.0 equiv) | | 2.0 equiv) | | | | |
| 57 | 2-(2-chloroethyl)-1- | intermediate 3 | NaI | EtOH | 80 | 4 | h |
| | methylpyrrolidine | (150 mg, 1.0 equiv) | (234 mg, | | | | |
| | hydrochloride | | 2.0 equiv) | | | | |
| | (287 mg, 2.0 equiv) | | | | | | |
| 58 | 1-(2-bromoethyl)azepane | intermediate 3 | NaI | MeCN | 80 | 18 | h |
| | hydrobromide | (70 mg, 1.0 equiv) | (109 mg, | | | | |
| | (209 mg, 2.0 equiv) | | 2.0 equiv) | | | | |
| 59 | 1-(2-chloroethyl)pyrrolidine | intermediate 4 | NaI | MeCN | 80 | 32 | h |
| | hydrochloride | (100 mg, 1.0 equiv) | (151 mg, | | | | |
| | (171 mg, 2.0 equiv) | | 2.0 equiv) | | | | |
| 60 | 1-(4-bromobutyl)pyrrolidine | intermediate 4 | NaI | MeCN | 80 | 5 | h |
| | hydrobromide | (70 mg, 1.0 equiv) | (106 mg, | | | | |
| | (202 mg, 2.0 equiv) | | 2.0 equiv) | | | | |
| 61 | 2-(bromomethyl)-4- | intermediate 3 | NaI | MeCN | 80 | 17 | h |
| | chlorothieno[3,2-c]pyridine | (66 mg, 1.0 equiv) | (51 mg, | | | | |
| | (90 mg, 1.0 equiv) | | 1.0 equiv) | | | | |
| 62 | intermediate 77 | intermediate 5 | — | MeCN | 80 | 16 | h |
| | (100 mg, 1.0 equiv) | (54 mg, 1.1 equiv) | | | | | |
| 63 | intermediate 78 | intermediate 5 | — | MeCN/EtOH | 80 | 18 | h |
| | (88 mg, 1.0 equiv) | (51 mg, 1.1 equiv) | | 3:1 | | | |
| 64 | intermediate 63 | intermediate 24 | — | MeCN/EtOH | 80 | 4 | d |
| | (50 mg, 1.0 equiv) | (51 mg, 1.1 equiv) | | 3:1 | | | |
| 65 | intermediate 62 | intermediate 5 | — | EtOH | 50 | 2 | d |
| | (90 mg, 1.0 equiv) | (54 mg, 1.1 equiv) | | | | | |
| 66 | intermediate 63 | intermediate 25 | — | DMA | 80 | 5 | d |
| | (100 mg, 1.0 equiv) | (87 mg, 1.1 equiv) | | | | | |
| 67 | intermediate 63 | intermediate 26 | — | DMA | 80 | 5 | d |
| | (100 mg, 1.0 equiv) | (87 mg, 1.1 equiv) | | | | | |
| 68 | intermediate 63 | intermediate 27 | — | MeCN/EtOH | 80 | 60 | h |
| | (100 mg, 1.0 equiv) | (74 mg, 1.8 equiv) | | 3:1 | | | |
| 69 | intermediate 79 | intermediate 5 | — | MeCN/EtOH | 80 | 3 | d |
| | (100 mg, 1.0 equiv) | (51 mg, 1.1 equiv) | | 3:1 | | | |
| 70 | intermediate 80 | intermediate 5 | — | MeCN/EtOH | 80 | 2 | d |
| | (100 mg, 1.0 equiv) | (51 mg, 1.1 equiv) | | 3:1 | | | |
| 71 | intermediate 84 | intermediate 5 | — | MeCN | 80 | 3 | d |
| | (200 mg, 2.0 equiv) | (75 mg, 1.0 equiv) | | | | | |
| 72 | intermediate 63 | intermediate 16 | — | MeCN/EtOH | 80 | 3 | d |
| | (100 mg, 1.0 equiv) | (69 mg, 1.1 equiv) | | 3:1 | | | |
| 73 | intermediate 70 | intermediate 12 | — | DMA | 80 | 2 | d |
| | (134 mg, 1.0 equiv) | (97 mg, 1.1 equiv) | | | | | |
| 74 | 1-(2-bromoethyl)-5-chloro- | intermediate 5 | NaI | MeCN | 80 | 3 | d |
| | 1H-indole | (64 mg, 1.0 equiv) | (64 mg, | | | | |
| | (100 mg, 1.0 equiv) | | 1.0 equiv) | | | | |
| 75 | intermediate 63 | intermediate 12 | — | DMA | 80 | 2 | d |
| | (100 mg, 1.0 equiv) | (68 mg, 1.1 equiv) | | | | | |
| 76 | intermediate 63 | intermediate 13 | — | DMA | 80 | 2 | d |
| | (100 mg, 1.0 equiv) | (68 mg, 1.1 equiv) | | | | | |
| 77 | 1-(2-chloroethyl)pyrrolidine | intermediate 5 | NaI | MeCN | 80 | 36 | h |
| | hydrochloride | (97 mg, 1.0 equiv) | (88 mg, | | | | |
| | (100 mg, 1.0 equiv) | | 1.0 equiv) | | | | |
| 78 | 1-(2-chloroethyl)pyrrolidine | intermediate 12 | NaI | MeCN | 80 | 3 | d |
| | hydrochloride | (113 mg, 1.0 equiv) | (88 mg, | | | | |
| | (100 mg, 1.0 equiv) | | 1.0 equiv) | | | | |
| 79 | intermediate 87 | intermediate 5 | — | MeCN/EtOH | 80 | 6 | d |
| | (27 mg, 1.0 equiv) | (13 mg, 1.1 equiv) | | 3:1 | | | |
| 80 | intermediate 88 | intermediate 11 | — | DMA | 80 | 2 | d |
| | (45 mg, 1.0 equiv) | (28 mg, 1.1 equiv) | | | | | |
| 81 | intermediate 89 | intermediate 5 | — | MeCN/EtOH | 80 | 16 | h |
| | (75 mg, 1.0 equiv) | (42 mg, 1.1 equiv) | | 3:1 | | | |
| 82 | intermediate 90 | intermediate 5 | — | MeCN/EtOH | 80 | 2 | d |
| | (100 mg, 1.0 equiv) | (53 mg, 1.0 equiv) | | 3:1 | | | |
| 83 | intermediate 88 | intermediate 39 | — | DMA | 80 | 16 | h |
| | (70 mg, 1.0 equiv) | (43 mg, 1.1 equiv) | | | | | |
| 84 | intermediate 91 | intermediate 5 | — | MeCN/EtOH | 80 | 16 | h |
| | (95 mg, 1.0 equiv) | (56 mg, 1.0 equiv) | | 3:1 | | | |
| 85 | intermediate 92 | intermediate 5 | — | MeCN/EtOH | 80 | 16 | h |
| | (60 mg, 1.0 equiv) | (29 mg, 1.1 equiv) | | 3:1 | | | |
| 86 | intermediate 93 | intermediate 5 | — | DMA | 80 | 16 | h |
| | (75 mg, 1.0 equiv) | (39 mg, 1.1 equiv) | | | | | |
| 87 | intermediate 62 | intermediate 12 | — | EtOH | 50 | 16 | h |
| | (100 mg, 1.0 equiv) | (70 mg, 1.1 equiv) | | | | | |
| 88 | intermediate 62 | intermediate 13 | — | EtOH | 50 | 16 | h |
| | (100 mg, 1.0 equiv) | (65 mg, 1.1 equiv) | | | | | |
| 89 | intermediate 62 | intermediate 34 | — | EtOH | 50 | 18 | h |
| | (80 mg, 1.0 equiv) | (48 mg, 1.1 equiv) | | | | | |
| 90 | 1-(2-bromoethyl)azepane | intermediate 5 | NaI | MeCN | 80 | 18 | h |
| | hydrobromide | (60 mg, 1.0 equiv) | (110 mg, | | | | |
| | (210 mg, 2.0 equiv) | | 2.0 equiv) | | | | |
| 91 | 1-(2-chloroethyl)piperidine | intermediate 5 | NaI | MeCN | 80 | 18 | h |
| | hydrochloride | (70 mg, 1.0 equiv) | (128 mg, | | | | |
| | (157 mg, 2.0 equiv) | | 2.0 equiv) | | | | |
| 92 | intermediate 88 | intermediate 42 | — | DMA | 80 | 4 | d |
| | (75 mg, 1.0 equiv) | (63 mg, 1.1 equiv) | | | | | |
| 93 | intermediate 62 | intermediate 55 | — | EtOH | 50 | 3 | d |
| | (50 mg, 1.0 equiv) | (40 mg, 1.1 equiv) | | | | | |
| 94 | intermediate 94 | intermediate 5 | — | MeCN/EtOH | 80 | 18 | h |
| | (40 mg, 1.0 equiv) | (18 mg, 1.0 equiv) | | 3:1 | | | |
| 108 | 2-(chloromethyl)-1- | intermediate 5 | NaI | MeCN | 80 | 6 | d |
| | methylpyrrolidine | (70 mg, 1.0 equiv) | (128 mg, | | | | |
| | hydrochloride | | 2.0 equiv) | | | | |
| | (145 mg, 2.0 equiv) | | | | | | |
| 109 | intermediate 140 | intermediate 5 | — | MeCN/EtOH | 80 | 18 | h |
| | (0.16 mmol, 1.0 equiv) | (27 mg, 1.0 equiv) | | 3:1 | | | |
| 110 | intermediate 141 | intermediate 5 | — | EtOH | 50 | 18 | h |
| | (54 mg, 1.0 equiv) | (23 mg, 1.1 equiv) | | | | | |
| 111 | intermediate 142 | intermediate 5 | — | EtOH | 40 | 20 | h |
| | (78 mg, 1.0 equiv) | (42 mg, 1.1 equiv) | | | | | |
| 112 | intermediate 143 | intermediate 5 | — | EtOH | 50 | 20 | h |
| | (78 mg, 1.0 equiv) | (45 mg, 1.1 equiv) | | | | | |
| 113 | intermediate 62 | intermediate 104 | — | EtOH | 50 | 22 | h |
| | (75 mg, 1.0 equiv) | (49 mg, 1.1 equiv) | | | | | |
| 114 | intermediate 62 | intermediate 4 | — | EtOH | 50 | 70 | h |
| | (75 mg, 1.0 equiv) | (49 mg, 1.0 equiv) | | | | | |
| 115 | intermediate 146 | intermediate 5 | — | MeCN | 80 | 18 | h |
| | (135 mg, 1.0 equiv) | (42 mg, 1.1 equiv) | | | | | |
| 116 | 2-(bromomethyl)-4- | intermediate 5 | NaI | EtOH | 50 | 2 | h |
| | chlorothieno[3,2-c]pyridine | (50 mg, 1.0 equiv) | (91 mg, | | | | |
| | (160 mg, 2.0 equiv) | | 2.0 equiv) | | | | |
| 117 | intermediate 62 | intermediate 108 | — | EtOH | 50 | 4 | d |
| | (75 mg, 1.0 equiv) | (50 mg, 1.1 equiv) | | | | | |
| 118 | intermediate 62 | intermediate 30 | — | EtOH | 50 | 22 | h |
| | (70 mg, 1.0 equiv) | (51 mg, 1.1 equiv) | | | | | |
| 119 | intermediate 62 | intermediate 25 | — | EtOH | 50 | 11 | d |
| | (75 mg, 1.0 equiv) | (67 mg, 1.1 equiv) | | | | | |
| 120 | intermediate 147 | intermediate 5 | — | EtOH | 50 | 17 | h |
| | (90 mg, 1.0 equiv) | (57 mg, 1.1 equiv) | | | | | |
| 121 | intermediate 148 | intermediate 5 | — | EtOH | 50 | 18 | h |
| | (93 mg, 1.0 equiv) | (59 mg, 1.1 equiv) | | | | | |
| 122 | intermediate 149 | intermediate 5 | — | EtOH | 50 | 18 | h |
| | (141 mg, 1.0 equiv) | (91 mg, 1.1 equiv) | | | | | |
| 123 | intermediate 150 | intermediate 5 | — | EtOH | 50 | 18 | h |
| | (65 mg, 1.0 equiv) | (39 mg, 1.1 equiv) | | | | | |
| 124 | intermediate 151 | intermediate 5 | — | EtOH | 50 | 18 | h |
| | (86 mg, 1.0 equiv) | (53 mg, 1.2 equiv) | | | | | |
| 125 | intermediate 152 | intermediate 5 | NaI | MeCN | 80 | 36 | h |
| | (1.32 mmol, 1.0 equiv) | (218 mg, 1.0 equiv) | (199 mg, | | | | |
| | | | 1.0 equiv) | | | | |
| 126 | intermediate 153 | intermediate 5 | — | EtOH | 40 | 18 h | |
| | (74 mg, 1.0 equiv) | (46 mg, 1.2 equiv) | | | then | then | |
| | | | | | 50 | 3 h | |
| 127 | intermediate 156 | intermediate 5 | NaI | MeCN | 80 | 36 | h |
| | (108 mg, 1.0 equiv) | (89 mg, 1.0 equiv) | (81 mg, | | | | |
| | | | 1.0 equiv) | | | | |
| 128 | intermediate 159 | intermediate 5 | NaI | MeCN | 80 | 20 | h |
| | (0.60 mmol, 1.0 equiv) | (98 mg, 1.0 equiv) | (89 mg, | | | | |
| | | | 1.0 equiv) | | | | |
| 129 | intermediate 160 | intermediate 5 | NaI | MeCN | 80 | 3 | d |
| | (235 mg, 1.0 equiv) | (210 mg, 1.0 equiv) | (191 mg, | | | | |
| | | | 1.0 equiv) | | | | |
| 130 | intermediate 163 | intermediate 5 | NaI | MeCN | 80 | 2 | d |
| | (158 mg, 1.0 equiv) | (141 mg, 1.0 equiv) | (129 mg, | | | | |
| | | | 1.0 equiv) | | | | |
| 131 | 1-(2-chloroethyl)pyrrolidine | intermediate 16 | NaI | MeCN | 80 | 7 | h |
| | hydrochloride | (54 mg, 1.0 equiv) | (42 mg, | | | | |
| | (48 mg, 1.0 equiv) | | 1.0 equiv) | | | | |
| 132 | intermediate 167 | intermediate 5 | NaI | MeCN | 80 | 18 | h |
| | (1.77 mmol, 1.0 equiv) | (167 mg, 1.0 equiv) | (265 mg, | | | | |
| | | | 1.0 equiv) | | | | |
| 133 | intermediate 170 | intermediate 5 | NaI | MeCN | 80 | 18 | h |
| | (2.03 mmol, 1.0 equiv) | (235 mg, 1.0 equiv) | (257 mg, | | | | |
| | | | 1.0 equiv) | | | | |
| 134 | 1-(2-chloroethyl)pyrrolidine | intermediate 132 | NaI | DMA | 120 | 6 | h |
| | hydrochloride | (218 mg, 1.0 equiv) | (904 mg, | | | | |
| | (1026 mg, 15 equiv) | | 15 equiv) | | | | |
| 135 | 1-(2-chloroethyl)pyrrolidin-2- | intermediate 5 | NaI | MeCN | 80 | 3 | d |
| | one | (167 mg, 1.0 equiv) | (152 mg, | | | | |
| | (150 mg, 1.0 equiv) | | 1.0 equiv) | | | | |
| 136 | 1-(3-chloropropyl)pyrrolidine | intermediate 5 | NaI | MeCN | 80 | 36 | h |
| | hydrochloride | (112 mg, 1.0 equiv) | (102 mg, | | | | |
| | (125 mg, 1.0 equiv) | | 1.0 equiv) | | | | |
| 137 | 1-(4-bromobutyl)pyrrolidine | intermediate 5 | NaI | MeCN | 80 | 36 | h |
| | hydrobromide | (114 mg, 1.0 equiv) | (104 mg, | | | | |
| | (200 mg, 1.0 equiv) | | 1.0 equiv) | | | | |
| 138 | intermediate 171 | intermediate 5 | NaI | MeCN | 80 | 2 | d |
| | (1.16 mmol, 1.0 equiv) | (191 mg, 1.0 equiv) | (174 mg, | | | | |
| | | | 1.0 equiv) | | | | |
| 139 | intermediate 174 | intermediate 5 | NaI | MeCN | 80 | 4 | d |
| | (102 mg, 1.0 equiv) | (85 mg, 1.0 equiv) | (77 mg, | | | | |
| | | | 1.0 equiv) | | | | |
| 140 | intermediate 177 | intermediate 5 | NaI | MeCN | 80 | 3 | d |
| | (158 mg, 1.0 equiv) | (105 mg, 1.0 equiv) | (96 mg, | | | | |
| | | | 1.0 equiv) | | | | |
| 141 | intermediate 178 | intermediate 5 | NaI | MeCN | 80 | 18 | h |
| | (0.64 mmol, 1.0 equiv) | (105 mg, 1.0 equiv) | (96 mg, | | | | |
| | | | 1.0 equiv) | | | | |
| 142 | intermediate 181 | intermediate 5 | NaI | MeCN | 80 | 36 | h |
| | (188 mg, 1.0 equiv) | (138 mg, 1.0 equiv) | (126 mg, | | | | |
| | | | 1.0 equiv) | | | | |
| 143 | intermediate 184 | intermediate 5 | NaI | MeCN | 80 | 5 | d |
| | (223 mg, 1.0 equiv) | (133 mg, 1.0 equiv) | (121 mg, | | | | |
| | | | 1.0 equiv) | | | | |
| 144 | intermediate 187 | intermediate 5 | NaI | MeCN | 80 | 4 | d |
| | (311 mg, 1.0 equiv) | (262 mg, 1.0 equiv) | (239 mg, | | | | |
| | | | 1.0 equiv) | | | | |
| 145 | intermediate 188 | intermediate 5 | NaI | MeCN | 80 | 5 | d |
| | (1.50 mmol, 1.0 equiv) | (246 mg, 1.0 equiv) | (225 mg, | | | | |
| | | | 1.0 equiv) | | | | |
| 146 | intermediate 191 | intermediate 5 | NaI | MeCN | 80 | 4 | d |
| | (140 mg, 1.0 equiv) | (116 mg, 1.0 equiv) | (106 mg, | | | | |
| | | | 1.0 equiv) | | | | |
| 147 | 4-chloro-1-(pyrrolidin-1- | intermediate 5 | NaI | MeCN | 80 | 18 | h |
| | yl)butan-1-one | (94 mg, 1.0 equiv) | (85 mg, | | | | |
| | (100 mg, 1.0 equiv) | | 1.0 equiv) | | | | |
| 148 | intermediate 192 | intermediate 5 | NaI | MeCN | 80 | 18 | h |
| | (0.59 mmol, 1.0 equiv) | (97 mg, 1.0 equiv) | (89 mg, | | | | |
| | | | 1.0 equiv) | | | | |
| 149 | 1-(4-bromobutyl)pyrrolidine | intermediate 11 | NaI | MeCN | 80 | 18 | h |
| | hydrobromide | (64 mg, 1.0 equiv) | (52 mg, | | | | |
| | (100 mg, 1.0 equiv) | | 1.0 equiv) | | | | |
| 150 | 1-(4-bromobutyl)pyrrolidine | intermediate 6 | NaI | MeCN | 80 | 18 | h |
| | hydrobromide | (69 mg, 1.0 equiv) | (52 mg, | | | | |
| | (100 mg, 1.0 equiv) | | 1.0 equiv) | | | | |
| 151 | 1-(4-bromobutyl)pyrrolidine | intermediate 34 | NaI | MeCN | 80 | 18 | h |
| | hydrobromide | (61 mg, 1.0 equiv) | (50 mg, | | | | |
| | (96 mg, 1.0 equiv) | | 1.0 equiv) | | | | |
| 152 | 1-(4-bromobutyl)pyrrolidine | intermediate 39 | NaI | MeCN | 80 | 18 | h |
| | hydrobromide | (58 mg, 1.0 equiv) | (48 mg, | | | | |
| | (91 mg, 1.0 equiv) | | 1.0 equiv) | | | | |
| 153 | 1-(4-bromobutyl)pyrrolidine | intermediate 135 | NaI | MeCN | 80 | 18 | h |
| | hydrobromide | (69 mg, 1.0 equiv) | (52 mg, | | | | |
| | (100 mg, 1.0 equiv) | | 1.0 equiv) | | | | |
| 154 | intermediate 196 | intermediate 5 | NaI | MeCN | 80 | 5 | d |
| | (1.29 mmol, 1.0 equiv) | (212 mg, 1.0 equiv) | (194 mg, | | | | |
| | | | 1.0 equiv) | | | | |
| 155 | intermediate 197 | intermediate 5 | NaI | MeCN | 80 | 4 | d |
| | (150 mg, 1.0 equiv) | (132 mg, 1.0 equiv) | (121 mg, | | | | |
| | | | 1.0 equiv) | | | | |
| 156 | intermediate 200 | intermediate 5 | NaI | MeCN | 80 | 4 | d |
| | (271 mg, 1.0 equiv) | (237 mg, 1.0 equiv) | (216 mg, | | | | |
| | | | 1.0 equiv) | | | | |
| 157 | intermediate 203 | intermediate 5 | NaI | MeCN | 80 | 4 | d |
| | (233 mg, 1.0 equiv) | (203 mg, 1.0 equiv) | (186 mg, | | | | |
| | | | 1.0 equiv) | | | | |
| 158 | 2-(2-chloroethyl)-1-methyl- | intermediate 4 | NaI | MeCN | 80 | 2 | d |
| | pyrrolidine hydrochloride | (100 mg, 1.0 equiv) | (152 mg, | | | | |
| | (186 mg, 2.0 equiv) | | 2.0 equiv) | | | | |
| 159 | 1-(4-bromobutyl)pyrrolidine | intermediate 13 | NaI | MeCN | 80 | 18 | h |
| | hydrobromide (97 mg, | (60 mg, 1.0 equiv) | (50 mg, | | | | |
| | 1.0 equiv) | | 1.0 equiv) | | | | |
| 160 | 1-(4-bromobutyl)pyrrolidine | intermediate 12 | NaI | MeCN | 80 | 18 | h |
| | hydrobromide (100 mg, | (70 mg, 1.0 equiv) | (55 mg, | | | | |
| | 1.0 equiv) | | 1.0 equiv) | | | | |
| 161 | 1-(3-chloropropyl)pyrrolidine | intermediate 4 | NaI | MeCN | 80 | 20 | h |
| | hydrochloride | (93 mg, 1.0 equiv) | (75 mg, | | | | |
| | (100 mg, 1.0 equiv) | | 1.0 equiv) | | | | |
| 162 | intermediate 207 | intermediate 4 | NaI | MeCN | 80 | 72 | h |
| | (107 mg, 1.0 equiv) | (100 mg, 1.0 equiv) | (75 mg, | | | | |
| | | | 1.0 equiv) | | | | |
| 163 | 1-(4-bromobutyl)pyrrolidine | intermediate 26 | NaI | MeCN | 80 | 20 | h |
| | hydrobromide | (200 mg, 1.0 equiv) | (123 mg, | | | | |
| | (236 mg, 1.0 equiv) | | 1.0 equiv) | | | | |
| 164 | 1-(4-bromobutyl)piperidine | intermediate 4 | NaI | MeCN | 80 | 20 | h |
| | hydrobromide | (90 mg, 1.0 equiv) | (140 mg, | | | | |
| | (270 mg, 1.0 equiv) | | 1.0 equiv) | | | | |
| 165 | intermediate 207 | intermediate 5 | NaI | MeCN | 80 | 72 | h |
| | (129 mg, 1.0 equiv) | (100 mg, 1.0 equiv) | (91 mg, | | | | |
| | | | 1.0 equiv) | | | | |
| 166 | intermediate 208 | Intermediate 4 | NaI | MeCN | 80 | 7 | d |
| | (278 mg, 3.0 equiv) | (100 mg, 1.0 equiv) | (227 mg, | | | | |
| | | | 3.0 equiv) | | | | |
| 167 & 168 | intermediate 209 | Intermediate 4 | NaI | MeCN | 80 | 6 | d |
| | (120 mg, 2.0 equiv) | (65 mg, 1.0 equiv) | (100 mg, | | | | |
| | | | 2.0 equiv) | | | | |
| 169 | 1-(4-bromobutyl)pyrrolidine | intermediate 17 | NaI | MeCN | 80 | 18 | h |
| | hydrobromide | (90 mg, 1.0 equiv) | (47 mg, | | | | |
| | (90 mg, 1.0 equiv) | | 1.0 equiv) | | | | |
| 170 | 1-(4-bromobutyl)pyrrolidine | intermediate 16 | NaI | MeCN | 80 | 18 | h |
| | hydrobromide | (62 mg, 1.0 equiv) | (48 mg, | | | | |
| | (93 mg, 1.0 equiv) | | 1.0 equiv) | | | | |
| 171 | 1-(4-bromobutyl)pyrrolidine | intermediate 7 | NaI | MeCN | 80 | 18 | h |
| | hydrobromide | (80 mg, 1.0 equiv) | (67 mg, | | | | |
| | (130 mg, 1.0 equiv) | | 1.0 equiv) | | | | |
| 172 | 1-(4-bromobutyl)pyrrolidine | 4,4- | NaI | MeCN | 80 | 18 | h |
| | hydrobromide | dimethylimidazolidine-2- | (92 mg, | | | | |
| | (180 mg, 1.0 equiv) | thione | 1.0 equiv) | | | | |
| | | (80 mg, 1.0 equiv) | | | | | |
| 173 | 1-(4-bromobutyl)pyrrolidine | tetrahydropyrimidine- | NaI | MeCN | 80 | 18 | h |
| | hydrobromide | 2(1H)-thione | (100 mg, | | | | |
| | (200 mg, 1.0 equiv) | (80 mg, 1.0 equiv) | 1.0 equiv) | | | | |
| 174 | intermediate 210 | intermediate 4 | NaI | MeCN | 80 | 48 | h |
| | (1.05 mmol, 1.8 equiv) | (115 mg, 1.0 equiv) | (100 mg, | | | | |
| | | | 2.0 equiv) | | | | |
| 175 | 1-(4-bromobutyl)pyrrolidine | imidazolidine-2-thione | NaI | MeCN | 80 | 16 | h |
| | hydrobromide | (71 mg, 1.0 equiv) | (104 mg, | | | | |
| | (200 mg, 1.0 equiv) | | 1.0 equiv) | | | | |
| 176 | intermediate 211 | intermediate 4 | NaI | MeCN | 80 | 18 | h |
| | (0.86 mmol, 1.0 equiv) | (160 mg, 1.0 equiv) | (121 mg | | | | |
| | | | 1.0 equiv) | | | | |
| 177 | intermediate 188 | intermediate 4 | NaI | MeCN | 80 | 48 | h |
| | (213 mg, 2.0 equiv) | (115 mg, 1.0 equiv) | (174 mg, | | | | |
| | | | 2.0 equiv) | | | | |
| 178 | 1-(4-bromobutyl)pyrrolidine | 1,3-diazepane-2-thione | NaI | MeCN | 80 | 18 | h |
| | hydrobromide | (65 mg, 1.0 equiv) | (75 mg, | | | | |
| | (144 mg, 1.0 equiv) | | 1.0 equiv) | | | | |
| 179 | 1-(4-bromobutyl)pyrrolidine | intermediate 136 | NaI | MeCN | 80 | 18 | h |
| | hydrobromide | (70 mg, 1.0 equiv) | (73 mg, | | | | |
| | (140 mg, 1.0 equiv) | | 1.0 equiv) | | | | |
| 180 | 1-(4-bromobutyl)pyrrolidine | intermediate 137 | NaI | MeCN | 80 | 20 | h |
| | hydrobromide | (100 mg, 1.0 equiv) | (79 mg, | | | | |
| | (151 mg, 1.0 equiv) | | 1.0 equiv) | | | | |
| 181 | 1-(4-bromobutyl)pyrrolidine | intermediate 3 | NaI | MeCN | 80 | 18 | h |
| | hydrobromide | (120 mg, 1.0 equiv) | (93 mg. | | | | |
| | (179 mg, 1.0 equiv) | | 1.0 equiv) | | | | |
| 182 | 1-(2-chloroethyl)pyrrolidine | intermediate 13 | NaI | MeCN | 80 | 2 | d |
| | hydrochloride | (80 mg, 1.0 equiv) | (134 mg, | | | | |
| | (152 mg, 2.0 equiv) | | 2.0 equiv) | | | | |
| 183 | 1-(2-chloroethyl)pyrrolidine | intermediate 17 | NaI | MeCN | 80 | 20 | h |
| | hydrochloride | (100 mg, 1.0 equiv) | (63 mg, | | | | |
| | (72 mg, 1.0 equiv) | | 1.0 equiv) | | | | |
| 184 | 1-(2-chloroethyl)pyrrolidine | intermediate 138 | NaI | MeCN | 80 | 20 | h |
| | hydrochloride | (83 mg, 1.0 equiv) | (73 mg, | | | | |
| | (83 mg, 1.0 equiv) | | 1.0 equiv) | | | | |
| 185 | 1-(4-bromobutyl)pyrrolidine | intermediate 27 | NaI | MeCN | 80 | 20 | h |
| | hydrobromide | (100 mg, 1.0 equiv) | (117 mg, | | | | |
| | (224 mg, 1.0 equiv) | | 1.0 equiv) | | | | |
| 186 | 1-(2-chloroethyl)pyrrolidine | intermediate 7 | NaI | MeCN | 80 | 18 | h |
| | hydrochloride | (160 mg, 1.0 equiv) | (168 mg, | | | | |
| | (191 mg, 1.3 equiv) | | 1.3 equiv) | | | | |
| 187 | 1-(2-chloroethyl)pyrrolidine | intermediate 27 | NaI | MeCN | 80 | 2 | d |
| | hydrochloride | (120 mg, 2.0 equiv) | (280 mg, | | | | |
| | (318 mg, 1.0 equiv) | | 2.0 equiv) | | | | |
| 188 | 4-(bromomethyl)pyridine | intermediate 13 | NaI | MeCN | 80 | 16 | h |
| | hydrobromide | (100 mg, 1.0 equiv) | (84 mg, | | | | |
| | (142 mg, 1.0 equiv) | | 1.0 equiv) | | | | |
| 189 | 4-(bromomethyl)pyridine | intermediate 7 | NaI | MeCN | 80 | 6 | h |
| | hydrobromide | (100 mg, 1.0 equiv) | (84 mg, | | | | |
| | (142 mg, 1.0 equiv) | | 1.0 equiv) | | | | |
| 190 | N-(3-chloropropyl)pyrrolidine | intermediate 13 | NaI | MeCN | 80 | 48 | h |
| | hydrochloride | (80 mg, 1.0 equiv) | (130 mg, | | | | |
| | (170 mg, 2.0 equiv) | | 2.0 equiv) | | | | |
| 191 | intermediate 213 | intermediate 13 | — | MeCN | 80 | 20 | h |
| | (139 mg, 1.0 equiv) | (86 mg, 1.0 equiv) | | | | | |
| 192 | intermediate 146 | intermediate 13 | — | MeCN | 80 | 20 | h |
| | (197 mg, 1.0 equiv) | (129 mg, 1.0 equiv) | | | | | |
| 193 | 3-(chloromethyl)pyridine | intermediate 7 | NaI | MeCN | 80 | 6 | h |
| | hydrochloride | (70 mg, 1.0 equiv) | (70 mg, | | | | |
| | (80 mg, 1.2 equiv) | | 1.2 equiv) | | | | |
| 194 | N-(3-chloropropyl)pyrrolidine | intermediate 7 | NaI | MeCN | 80 | 48 | h |
| | hydrochloride | (80 mg, 1.0 equiv) | (67 mg, | | | | |
| | (83 mg, 1.0 equiv) | | 1.0 equiv) | | | | |
| 195 | intermediate 146 | intermediate 7 | — | MeCN | 80 | 20 | h |
| | (153 mg, 1.0 equiv) | (100 mg, 1.0 equiv) | | | | | |
| 196 | intermediate 164 | intermediate 5 | — | EtOH | 50 | 18 | h |
| | (145 mg, 1.0 equiv) | (82 mg, 1.2 equiv) | | | | | |
| 197 | (2-chloroethyl)cyclopentane | intermediate 5 | NaI | MeCN | 80 | 36 | h |
| | (130 mg, 1.0 equiv) | (161 mg, 1.0 equiv) | (147 mg, | | | | |
| | | | 1.0 equiv) | | | | |

The following examples of the invention were prepared according to general procedure B using the reaction conditions detailed in the following table, and isolated as described hereinafter.

| Exam- | Electrophile | Thiourea | Conditions | Conditions |
|---|---|---|---|---|
| ple | (mass, equiv) | (mass, equiv) | of Step 1 | of Step 2 |
| 95 | intermediate 75 | intermediate 5 | MeCN, 80° C. | 80° C. |
| | (90 mg, 1.0 | (64 mg, 1.1 | 22 h | 6 d |
| | equiv) | equiv) | | |
| 96 | intermediate 81 | intermediate 5 | MeCN, 80° C. | 110° C. |
| | (140 mg, 1.0 | (91 mg, 1.1 | 16 h | 2 d |
| | equiv) | equiv) | | |
| 97 | intermediate 77 | intermediate 5 | MeCN, 80° C. | 110° C. |
| | (100 mg, 1.0 | (53 mg, 1.1 | 16 h | 48 h |
| | equiv) | equiv) | | |

The following examples of the invention were prepared according to general procedure C using the reaction conditions detailed in the following table, and isolated as described hereinafter.

| | Electrophile | Thiourea | | | | | |
|---|---|---|---|---|---|---|---|
| Example | (mass, equiv) | (mass, equiv) | Base | Solvent | T ° C. | time | |
| 98 | Intermediate 85 | intermediate 12 | NaH 60% | THF | 70 | 18 | h |
| | (157 mg, 1.0 equiv) | (168 mg, 1.0 equiv) | (39 mg, 1.1 equiv) | | | | |
| 99 | intermediate 62 | intermediate 50 | NaH 60% | THF | rt | 4 | h |
| | (80 mg, 1.0 equiv) | (59 mg, 1.0 equiv) | (22 mg, 2.0 equiv) | | | | |
| 100 | Intermediate 85 | intermediate 5 | NaH 60% | THF | 70 | 2 | d |
| | (177 mg, 1.0 equiv) | (162 mg, 1.0 equiv) | (44 mg, 1.1 equiv) | | | | |
| 101 | intermediate 193 | intermediate 5 | NaH 60% | THF | 70 | 3 | d |
| | (0.58 mmol, 1.0 equiv) | (95 mg, 1.0 equiv) | (26 mg, 1.1 equiv) | | | | |
| 102 | intermediate 204 | intermediate 5 | NaH 60% | THF | 70 | 19 | h |
| | (0.63 mmol, 1.0 equiv) | (103 mg, 1.0 equiv) | (40 mg, 1.6 equiv) | | | | |
| 103 | intermediate 85 | intermediate 4 | NaH 60% | THF | 70 | 3 | d |
| | (71 mg, 1.0 equiv) | (79 mg, 1.0 equiv) | (17 mg, 1.1 equiv) | | | | |
| 104 | intermediate 205 | intermediate 5 | NaH 60% | THF | 70 | 3 | d |
| | (125 mg, 1.0 equiv) | (118 mg, 1.1 equiv) | (26 mg, 1.0 equiv) | | | | |
| 105 | intermediate 85 | intermediate 13 | NaH 60% | THF | 80 | 40 | h |
| | (151 mg, 1.0 equiv) | (160 mg, 1.1 equiv) | (37 mg, 1.1 equiv) | | | | |
| 106 | intermediate 193 | intermediate 7 | NaH 60% | THF | 70 | 18 | h |
| | (0.47 mmol, 1.0 equiv) | (120 mg, 1.0 equiv) | (26 mg, 1.1 equiv) | | | | |
| 107 | intermediate 193 | intermediate 13 | NaH 60% | THF | 80 | 20 | h |
| | (0.42 mmol, 1.0 equiv) | (101 mg, 1.0 equiv) | (17 mg, 1.0 equiv) | | | | |

### Example 1: 3-(((6-chloro-1,4-dihydroquinazolin-2-yl)thio)methyl)-6,6-dimethyl-5,6-dihydroimidazo[2,1-b]thiazole dihydrochloride

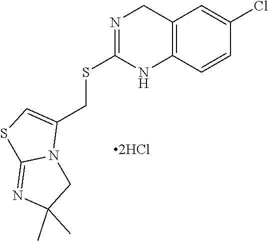 [SMILES: CC1(C)CN2C(CSC3=NCc4cc(Cl)ccc4N3)=CSC2=N1]

Example 1 was isolated as a white solid (156 mg, 85%) by centrifugation of the reaction mixture followed by trituration of the solid in MeCN (2×2 mL) and in Et₂O (2×2 mL).

¹H-NMR (DMSO-d₆+D₂O, 400 MHz) δ: 1.49 (s, 6H, 2 CH₃); 4.23 (s, 2H, S—CH₂); 4.65 (s, 2H, N—CH₂); 4.71 (s, 2H, N—CH₂); 6.95 (s, 1H, S—CH); 7.20-7.22 (d, J 8.6 Hz, 1H, Ar); 7.33 (d, J 2.3 Hz, 1H, Ar); 7.38 (dd, J 8.6; 2.3 Hz, 1H, Ar). M/Z (M[³⁵Cl]+H)⁺: 365.1. Mp>250° C.

### Example 2: 7-chloro-3-(((5,5-dimethyl-4,5-dihydro-1H-imidazol-2-yl)thio)methyl)-5H-thiazolo[2,3-b]quinazoline dihydrochloride

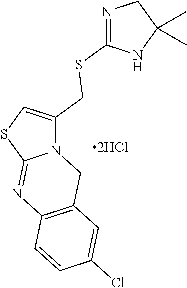 [SMILES: CC1(C)CN=C(SCC2=CSC3=Nc4ccc(Cl)cc4CN23)N1]

Example 2 was isolated as a white solid (115 mg, 81%) by centrifugation of the reaction mixture followed by trituration of the solid in MeCN (2×2 mL) and in Et₂O (2×2 mL).

¹H-NMR (DMSO-d₆+D₂O, 400 MHz) δ: 1.33 (s, 6H, 2 CH₃); 3.61 (s, 2H, S—CH₂); 4.56 (s, 2H, N—CH₂); 5.40 (s, 2H, N—CH₂); 7.01 (d, J 8.5 Hz, 1H, Ar); 7.20 (s, 1H, S—CH); 7.35 (d, J 2.2 Hz, 1H, Ar); 7.39 (dd, J 8.5, 2.2 Hz, 1H, Ar). M/Z (M[³⁵Cl]+H)⁺: 365.1. Mp>250° C.

### Example 3: 3-(((3,4-dihydroquinazolin-2-yl)thio)methyl)-6,6-dimethyl-5,6-dihydroimidazo[2,1-b]thiazole dihydrochloride

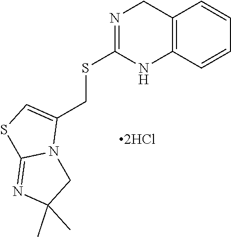 [SMILES: CC1(C)CN2C(CSC3=NCc4ccccc4N3)=CSC2=N1]

Example 3 was isolated as a white solid (140 mg, 83%) by centrifugation of the reaction mixture followed by trituration of the solid in MeCN (2×2 mL) and in Et₂O (2×2 mL).

¹H-NMR (DMSO-d₆, 400 MHz) δ: 1.51 (s, 6H, 2 CH₃); 4.30 (s, 2H, S—CH₂); 4.66 (s, 2H, N—CH₂); 4.93 (s, 2H, N—CH₂—Ar); 7.05 (s, 1H, S—CH); 7.22-7.33 (m, 4H, 4 Ar); 10.42 (bs, 1H, HCl salt); 11.43 (bs, 1H, HCl salt); 13.04 (bs, 1H, NH). M/Z (M+H)⁺: 331.1. Mp: 245-249° C.

### Example 4: 7-chloro-3-(((4,5-dihydro-1H-imidazol-2-yl)thio)methyl)-5H-thiazolo[2,3-b]quinazoline dihydrochloride

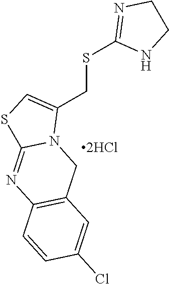 [SMILES: Clc1ccc2c(c1)CN1C(CSC3=NCCN3)=CSC1=N2]

Example 4 was isolated as a white solid (120 mg, 90%) by centrifugation of the reaction mixture followed by trituration of the solid in MeCN (3 mL) and in Et₂O (3 mL).

¹H-NMR (DMSO-d₆+D₂O, 400 MHz) δ: 3.85 (s, 4H, 2 N—CH₂); 4.57 (s, 2H, S—CH₂); 5.41 (s, 2H, N—CH₂—Ar); 7.01 (d, J 8.4 Hz, 1H, Ar); 7.23 (s, 1H, S—CH₂); 7.36-7.40 (m, 2H, 2 Ar). M/Z (M³⁵[Cl]+H)⁺: 337.0. Mp>250° C.

### Example 5: 3-(((5,5-dimethyl-4,5-dihydro-1H-imidazol-2-yl)thio)methyl)-5H-thiazolo[2,3-b]quinazoline dihydrochloride

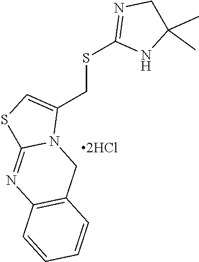 [SMILES: CC1(C)CN=C(SCC2=CSC3=Nc4ccccc4CN23)N1]

Example 5 was isolated as a white solid (130 mg, 88%) by centrifugation of the reaction mixture followed by trituration of the solid in MeCN (2×2 mL) and in Et₂O (2×2 mL).

¹H-NMR (DMSO-d₆+D₂O, 400 MHz) δ: 1.36 (s, 6H, 2 CH₃); 3.62 (s, 2H, S—CH₂); 4.73 (s, 2H, N—CH₂); 5.49 (s, 2H, N—CH₂); 7.05 (d, J 7.2 Hz, 1H, Ar); 7.20-7.28 (m, 2H, 2 Ar); 7.34-7.38 (m, 2H, 2 Ar+S-CH). M/Z (M+H)⁺: 331.1. Mp>250° C.

### Example 6: 3-(((7-chloro-1,4-dihydroquinazolin-2-yl)thio)methyl)-6,6-dimethyl-5,6-dihydroimidazo[2,1-b]thiazole dihydrochloride

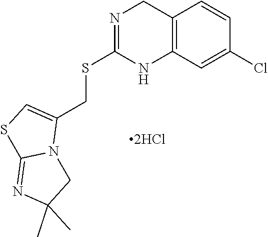 [SMILES: CC1(C)CN2C(CSC3=NCc4ccc(Cl)cc4N3)=CSC2=N1]

Example 6 was isolated as a white solid (140 mg, 90%) by centrifugation of the reaction mixture followed by trituration of the solid in MeCN (2×2 mL) and in Et₂O (2×2 mL).

¹H-NMR (DMSO-d₆, 400 MHz) δ: 1.51 (s, 6H, 2 CH₃); 4.28 (s, 2H, S—CH₂); 4.63 (s, 2H, N—CH₂); 4.92 (bs, 2H, N—CH₂); 7.05 (s, 1H, S—CH); 7.25-7.28 (m, 2H, 2 Ar); 7.45 (bs, 1H, Ar); 10.37 (s, 1H, HCl salt); 11.56 (bs, 1H, HCl salt); 13.31 (bs, 1H, NH). M/Z (M[³⁵Cl]+H)⁺: 365.1. Mp: 246-248° C.

### Example 7: 3-(((2,5-dihydro-1H-benzo[e][1,3]diazepin-3-yl)thio)methyl)-6,6-dimethyl-5,6-dihydroimidazo[2,1-b]thiazole dihydrochloride

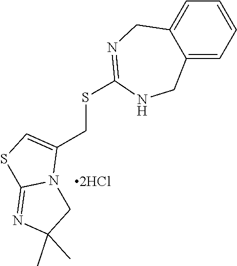 [SMILES: CC1(C)CN2C(CSC3=NCc4ccccc4CN3)=CSC2=N1]

Example 7 was isolated as a beige solid (125 mg, 72%) by centrifugation of the reaction mixture followed by trituration of the solid in MeCN (2×2 mL) and in Et₂O (2×2 mL), followed by precipitation with Et₂O from a solution in MeOH (0.5 mL) and freeze-drying in water.

¹H-NMR (DMSO-d₆, 400 MHz) δ: 1.46 (s, 6H, 2 CH₃); 4.20 (s, 2H, S—CH₂); 4.66 (s, 2H, N—CH₂); 4.73 (d, J4.4 Hz, 4H, N—CH₂); 6.82 (s, 1H, S—CH); 7.34-7.40 (m, 4H, 4 Ar); 10.34 (s, 1H, HCl salt); 10.84 (bs, 2H, NH+HCl salt). M/Z (M+H)⁺: 345.1. Mp: 127-134° C.

### Example 8: 3-(((5,5-dimethyl-4,5-dihydro-1H-imidazol-2-yl)thio)methyl)-5,10-dihydrobenzo[e]thiazolo[3,2-a][1,3]diazepine dihydrochloride

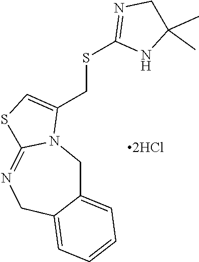 [SMILES: CC1(C)CN=C(SCC2=CSC3=NCc4ccccc4CN23)N1]

Example 8 was isolated as a white solid (120 mg, 83%) by centrifugation of the reaction mixture followed by trituration of the solid in MeCN (2×2 mL) and in Et₂O (2×2 mL).

¹H-NMR (DMSO-d₆+D₂O, 400 MHz) δ: 1.33 (s, 6H, 2 CH₃); 3.59 (s, 2H, S—CH₂); 4.69 (s, 2H, N—CH₂); 4.85 (s, 2H, N—CH₂); 5.47 (s, 2H, N—CH₂); 7.05 (s, 1H, S—CH); 7.42-7.47 (m, 3H, 3 Ar); 7.60-7.62 (m, 1H, Ar). M/Z (M+H)⁺: 345.1. Mp: 244-247° C.

### Example 9: 8-chloro-3-(((5,5-dimethyl-4,5-dihydro-1H-imidazol-2-yl)thio)methyl)-5H-thiazolo[2,3-b]quinazoline dihydrochloride

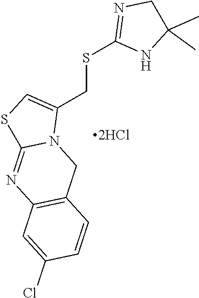 [SMILES: CC1(C)CN=C(SCC2=CSC3=Nc4cc(Cl)ccc4CN23)N1]

Example 9 was isolated as a white solid (130 mg, 91%) by centrifugation of the reaction mixture followed by trituration of the solid in MeCN (2×2 mL) and in Et₂O (2×2 mL).

¹H-NMR (DMSO-d₆, 400 MHz) δ: 1.37 (s, 6H, 2 CH₃); 3.61 (s, 2H, S—CH₂); 4.85 (s, 2H, N—CH₂); 5.49 (s, 2H, N—CH₂); 7.12 (s, 1H, S—CH); 7.27 (s, 2H, 2 Ar); 7.47 (bs, 1H, Ar); 10.64 (bs, 1H, HCl salt); 11.22 (bs, 1H, HCl salt); 13.77 (bs, 1H, NH). M/Z (M[³⁵Cl]+H)⁺: 365.1. Mp>250° C.

### Example 10: 3-(((6-chloro-1,4-dihydroquinazolin-2-yl)thio)methyl)-5,6-dihydroimidazo[2,1-b]thiazole dihydrochloride

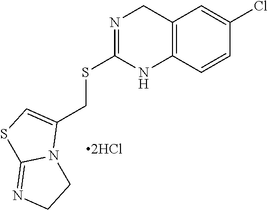 [SMILES: Clc1ccc2c(c1)CN=C(SCC1=CSC3=NCCN13)N2]

Example 10 was isolated as a white solid (12 mg, 8%) by centrifugation of the reaction mixture followed by trituration of the solid in EtOH (3 mL), in MeOH (3 mL), recrystallization from MeOH (2.5 mL) and freeze-drying in water.

¹H-NMR (DMSO-d₆+D₂O, 400 MHz) δ: 4.24-4.30 (m, 2H, N—CH₂); 4.38-4.43 (m, 2H, N—CH₂); 4.47 (s, 2H, N—CH₂—Ar); 4.62 (s, 2H, S—CH₂); 6.78 (s, 1H, S—CH); 7.05 (d, J8.6 Hz, 1H, Ar); 7.26 (d, J2.2 Hz, 1H, Ar); 7.34 (d, J8.6, 2.2 Hz, 1H, Ar). M/Z (M[³⁵Cl]+H)⁺: 337.1. Mp: 195-200° C.

### Example 11: 3-(((3,4-dihydroquinazolin-2-yl)thio)methyl)-5,10-dihydrobenzo[e]thiazolo[3,2-a][1,3]diazepine dihydrochloride

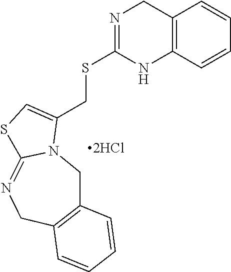 [SMILES: C1=C(CSC2=NCc3ccccc3N2)N2Cc3ccccc3CN=C2S1]

Example 11 was isolated as a grey solid (132 mg, 84%) by centrifugation of the reaction mixture followed by trituration of the solid in MeCN (3×2 mL), in Et₂O (2×2 mL) and freeze-drying in water.

¹H-NMR (DMSO-d₆+D₂O, 400 MHz) δ: 4.64 (s, 2H, S—CH₂); 4.67 (s, 2H, N—CH₂—Ar); 4.80 (s, 2H, N—CH₂—Ar); 5.46 (s, 2H, N—CH₂—Ar); 6.94 (s, 1H, S—CH); 7.06 (d, J7.6 Hz, 1H, Ar); 7.16 (d, J7.4 Hz, 1H, Ar); 7.23 (t, J7.4 Hz, 1H, Ar); 7.30 (t, J7.6 Hz, 1H, Ar); 7.39-7.46 (m, 3H, 3 Ar); 7.59-7.62 (m, 1H, Ar). M/Z (M+H)⁺: 379.1. Mp: 189-193° C.

### Example 12: 3-(((1,4-dihydroquinazolin-2-yl)thio)methyl)-5H-thiazolo[2,3-b]quinazoline dihydrochloride

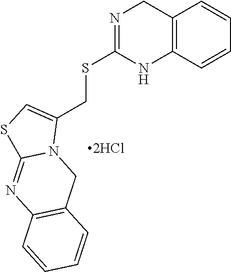 [SMILES: C1=C(CSC2=NCc3ccccc3N2)N2Cc3ccccc3N=C2S1]

Example 12 was isolated as a beige solid (145 mg, 91%) by centrifugation of the reaction mixture followed by trituration of the solid in MeCN (2×2 mL) and in Et₂O (3 mL).

¹H-NMR (DMSO-d₆, 400 MHz) δ: 4.68 (s, 2H, S—CH₂); 5.02 (s, 2H, CH₂—Ar); 5.59 (s, 2H, CH₂—Ar); 7.09 (d, J 8.0 Hz, 1H, Ar); 7.21-7.39 (m, 7H, 7 Ar); 7.43 (s, 2H, S—CH); 11.29 (bs, 1H, HCl salt); 12.89 (bs, 1H, HCl salt); 13.53 (bs, 1H, NH). M/Z (M+H)⁺: 365.1. Mp>250° C.

### Example 13: trans-3-(((3,4-dihydroquinazolin-2-yl)thio)methyl)-4a,5,6,7,8,8a-hexahydrobenzo[4,5]imidazo[2,1-b]thiazole dihydrochloride

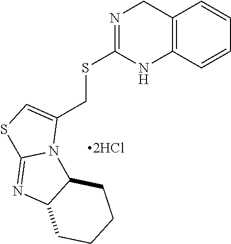

Example 13 was isolated as a white solid (126 mg, 78%) by centrifugation of the reaction mixture followed by trituration of the solid in MeCN (3×2 mL) and in Et₂O (3 mL).

¹H-NMR (DMSO-d₆, 400 MHz) δ: 1.33-1.47 (m, 2H, CH₂); 1.62-1.71 (m, 1H, CHₐH_{b}); 1.80-1.94 (m, 3H, CH₂+CHₐH_{b}); 2.22-2.25 (m, 1H, CHₐH_{b}); 2.63-2.66 (m, 1H, CHₐH_{b}); 4.02 (ddd, J 14.4, 11.2, 3.0 Hz, 1H, N—CH); 4.23 (ddd, J 14.4, 11.2, 3.0 Hz, 1H, N—CH); 4.65 (m, 2H, S—CH₂); 4.88 (d, J15.6 Hz, 1H, N—CHₐH_{b}—Ar); 5.01 (d, J15.6 Hz, 1H, N—CHₐH_{b}—Ar); 7.17 (s, 1H, S—CH); 7.21-7.27 (m, 2H, 2 Ar); 7.31-7.36 (m, 2H, 2 Ar); 10.33 (bs, 1H, NH); 11.38 (bs, 1H, HCl Salt); 13.04 (bs, 1H, HCl salt). M/Z (M+H)⁺: 357.1. Mp: 240-244° C.

### Example 14: 6-(4-chlorophenyl)-3-(((3,4-dihydroquinazolin-2-yl)thio)methyl)-5,6-dihydroimidazo[2,1-b]thiazole dihydrochloride

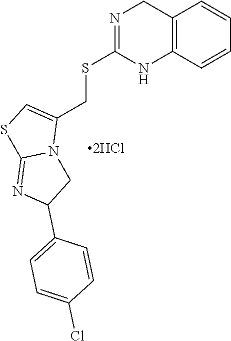 [SMILES: Clc1ccc(C2CN3C(CSC4=NCc5ccccc5N4)=CSC3=N2)cc1]

Example 14 was isolated as a white solid (125 mg, 83%) by centrifugation of the reaction mixture followed by trituration of the solid in MeCN (2×2 mL) and in Et₂O (2×2 mL).

¹H-NMR (DMSO-d₆, 400 MHz) δ: 4.36 (dd, J11.0, 8.5 Hz, 1H, N—CHₐH_{b}); 4.67 (s, 2H, S—CH₂); 4.82-4.92 (m, 2H, N—CH₂—Ar); 4.97 (t, J 11.0 Hz, 1H, N—CH); 5.90 (dd, J 11.0, 8.5 Hz, 1H, N—CHₐH_{b}); 7.12 (s, 1H, S—CH); 7.23-7.31 (m, 4H, 4 Ar); 7.52-7.54 (m, 4H, 4 Ar); 10.61 (bs, 1H, HCl salt); 11.27 (bs, 1H, HCl salt); 12.89 (bs, 1H, NH). M/Z (M[³⁵Cl]+H)⁺: 413.1. Mp: 225-230° C.

### Example 15: 6-cyclohexyl-3-(((3,4-dihydroquinazolin-2-yl)thio)methyl)-5,6-dihydroimidazo[2,1-b]thiazole dihydrochloride

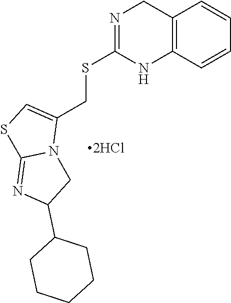 [SMILES: C1=C(CSC2=NCc3ccccc3N2)N2CC(C3CCCCC3)N=C2S1]

Example 15 was isolated as a white solid (124 mg, 80%) by centrifugation of the reaction mixture followed by trituration of the solid in MeCN (2×2 mL) and in Et₂O (2×2 mL).

¹H-NMR (DMSO-d₆, 400 MHz) δ: 0.92-1.27 (m, 5H, 2 CH₂+CH); 1.56-1.78 (m, 6H, 3 CH₂); 4.29-4.37 (m, 1H, N—CHₐH_{b}); 4.50-4.59 (m, 2H, N—CHₐH_{b}+N-CH); 4.66 (s, 2H, S—CH₂); 4.89 (bs, 2H, N—CH₂—Ar); 7.02 (s, 1H, S—CH); 7.2-7.34 (m, 4H, 4 Ar); 10.51 (bs, 1H, HCl salt); 11.32 (bs, 1H, HCl salt); 12.97 (bs, 1H, NH). M/Z (M+H)⁺: 385.2. Mp: 235-242° C.

### Example 16: trans-3-(((3,4-dihydroquinazolin-2-yl)thio)methyl)-5,6-diphenyl-5,6-dihydroimidazo[2,1-b]thiazole dihydrochloride

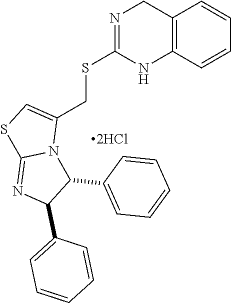 [SMILES: C1=C(CSC2=NCc3ccccc3N2)N2C(=N[C@H](c3ccccc3)[C@H]2c2ccccc2)S1]

Crude example 16 was obtained in a mixture with example 17 by centrifugation of the reaction mixture. The solid was triturated in MeCN (2×2 mL), in Et₂O (2×2 mL) and purified by preparative HPLC (column A, H₂O+0.1% HCOOH/MeCN+0.1% HCOOH 95:5 to 55:45). The fractions containing pure example 16 were freeze-dried with 1 N aqueous HCl to obtain a white solid (40 mg, 38%).

¹H-NMR (DMSO-d₆, 400 MHz) δ: 3.79 (d, J15.3 Hz, 1H, S—CHₐH_{b}); 4.61-4.63 (m, 3H, N—CH₂—Ar+S-CHₐH_{b}); 5.68 (d, J9.5 Hz, 1H, N—CH); 6.00 (d, J9.5 Hz, 1H, N—CH); 7.20 (s, 1H, S—CH); 7.22-7.34 (m, 4H, 4 Ar); 7.42-7.53 (m, 10H, 10 Ar); 11.19 (bs, 2H, 2 HCl salts); 12.87 (bs, 1H, NH). M/Z (M+H)⁺: 455.1. Mp: 175-185° C.

### Example 17: trans-3-(((3,4-dihydroquinazolin-2-yl)thio)methyl)-5,6-diphenyl-2,3,5,6-tetrahydroimidazo[2,1-b]thiazol-3-ol dihydrochloride

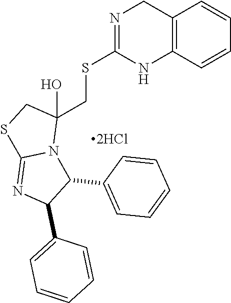 [SMILES: OC1(CSC2=NCc3ccccc3N2)CSC2=N[C@H](c3ccccc3)[C@@H](c3ccccc3)N21]

Crude example 17 was obtained in a mixture with example 16 by centrifugation of the reaction mixture. The solid was triturated in MeCN (2×2 mL), in Et₂O (2×2 mL) and purified by preparative HPLC (column A, H₂O+0.1% HCOOH/MeCN+0.1% HCOOH 95:5 to 55:45). The fractions containing pure example 17 were freeze-dried with 1 N aqueous HCl to obtain a white solid (18 mg, 12%).

¹H-NMR (DMSO-d₆, 400 MHz) δ: 4.07 (d, J 13.4 Hz, 1H, N—CH); 4.36-4.41 (m, 2H, N—CH₂—Ar); 4.50 (d, J 13.4 Hz, 1H, N—CH); 4.84-4.95 (m, 2H, 2 S—CHₐH_{b}); 5.09-5.11 (m, 1H, S—CHₐH_{b}); 5.63 (d, J9.6 Hz, 1H, S—CHₐH_{b}); 6.78-6.96 (m, 4H, 4 Ar); 7.18-7.52 (m, 10H, 10 Ar); 13.04 (bs, 2H, 2 HCl salts); OH and NH signals not observed. M/Z (M+H)⁺: 472.8. Mp: 195-200° C.

### Example 18: 3-(((3,4-dihydroquinazolin-2-yl)thio)methyl)-6-fluoro-5H-thiazolo[2,3-b]quinazoline dihydrochloride

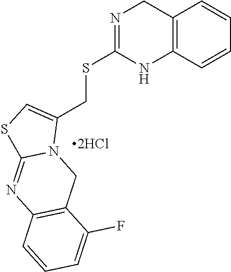 [SMILES: Fc1cccc2c1CN1C(CSC3=NCc4ccccc4N3)=CSC1=N2]

Crude example 18 was obtained by centrifugation of the reaction mixture. The solid was triturated in MeCN (2×2 mL), in Et₂O (2×2 mL), in hot MeOH (2×2 mL) and finally dissolved in H₂O (15 mL). The resulting aqueous layer was washed with DCM (2×10 mL) and freeze-dried to obtain a white solid (80 mg, 51%).

¹H-NMR (DMSO-d₆, 400 MHz) δ: 4.68 (s, 2H, S—CH₂); 4.99 (s, 2H, N—CH₂—Ar); 5.56 (s, 2H, N—CH₂—Ar); 6.91 (d, J8.5 Hz, 1H, Ar); 7.08 (t, J8.5 Hz, 1H, Ar); 7.23-7.27 (m, 3H, S-CH+2 Ar); 7.31-7.33 (m, 1H, Ar); 7.38-7.44 (m, 2H, 2 Ar); 11.12 (bs, 1H, HCl salt); 12.74 (bs, 1H, HCl salt); 13.70 (bs, 1H, NH). M/Z (M+H)⁺: 383.1. Mp: 193-203° C.

### Example 19: 7-chloro-3-(((1,4-dihydroquinazolin-2-yl)thio)methyl)-5H-thiazolo[2,3-b]quinazoline dihydrochloride

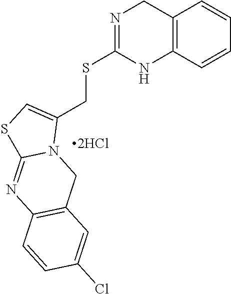 [SMILES: Clc1ccc2c(c1)CN1C(CSC3=NCc4ccccc4N3)=CSC1=N2]

Example 19 was isolated as a white solid (83 mg, 54%) by centrifugation of the reaction mixture followed by trituration of the solid in MeCN (3 mL), in Et₂O (3 mL) and in hot MeOH (2×2 mL).

¹H-NMR (DMSO-d₆+D₂O, 400 MHz) δ: 4.61 (s, 2H, S—CH₂); 4.70 (s, 2H, N—CH₂—Ar); 5.45 (s, 2H, N—CH₂—Ar); 7.00 (d, J 8.4 Hz, 1H, Ar); 7.05 (d, J7.8 Hz, 1H, Ar); 7.17-7.19 (m, 2H, S-CH+Ar); 7.31 (t, J7.6 Hz, 1H, Ar); 7.25 (t, J7.6 Hz, 1H, Ar); 7.36-7.39 (m, 2H, 2 Ar). M/Z (M[³⁵Cl]+H)⁺: 399.1. Mp>250° C.

### Example 20: 3-(((5-benzyl-4,5-dihydro-1H-imidazol-2-yl)thio)methyl)-7-chloro-5H-thiazolo[2,3-b]quinazoline dihydrochloride

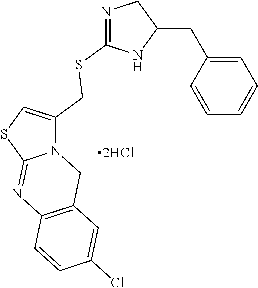 [SMILES: Clc1ccc2c(c1)CN1C(CSC3=NCC(Cc4ccccc4)N3)=CSC1=N2]

Example 20 was isolated as a white solid (139 mg, 86%) by centrifugation of the reaction mixture followed by trituration of the solid in MeCN (3 mL) and in Et₂O (3 mL).

¹H-NMR (DMSO-d₆, 400 MHz) δ: 2.88-2.98 (m, 2H, CH—CH₂—Ar); 3.60 (dd, J11.2, 6.8 Hz, 1H, N—CHₐH_{b}); 3.84 (t, J 11.2 Hz, 1H, N—CHₐH_{b}); 4.57-4.64 (m, 1H, N—CH); 4.78-4.87 (m, 2H, S—CH₂); 5.49 (s, 2H, N—CH₂—Ar); 7.12 (d, J 8.6 Hz, 1H, Ar); 7.27-7.38 (m, 7H, S-CH+7 Ar); 7.43 (dd, J8.6, 2.4 Hz, 1H, Ar); 10.70 (s, 1H, HCl salt); 11.06 (s, 1H, HCl Salt); 13.80 (bs, 1H, NH). M/Z (M[³⁵Cl]+H)⁺: 427.1. Mp>250° C.

### Example 21: 3-(((4,4-dimethyl-1,4-dihydroquinazolin-2-yl)thio)methyl)-5,10-dihydrobenzo[e]thiazolo[3,2-a][1,3]diazepine dihydrochloride

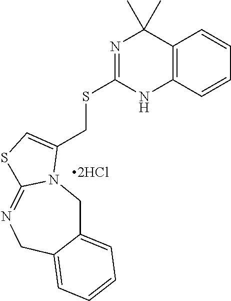 [SMILES: CC1(C)N=C(SCC2=CSC3=NCc4ccccc4CN23)Nc2ccccc21]

Example 21 was isolated as an off-white solid (125 mg, 75%) by centrifugation of the reaction mixture followed by trituration of the solid in EtOH (2×2 mL), in MeCN (2×2 mL) and in Et₂O (2×2 mL).

¹H-NMR (DMSO-d₆+D₂O, 400 MHz) δ: 1.59 (s, 6H, 2 CH₃); 4.87 (s, 2H, S—CH₂); 4.89 (s, 2H, N—CH₂—Ar); 5.58 (s, 2H, N—CH₂—Ar); 7.01 (s, 1H, S—CH); 7.18-7.21 (m, 1H, Ar); 7.26-7.30 (m, 1H, Ar); 7.32-7.39 (m, 2H, 2 Ar); 7.43-7.49 (m, 3H, 3 Ar); 7.68-7.71 (m, 1H, Ar). M/Z (M+H)⁺: 407.1. Mp>250° C.

### Example 22: 3-(((4-(4-chlorophenyl)-4,5-dihydro-1H-imidazol-2-yl)thio)methyl)-5,10-dihydrobenzo[e]thiazolo[3,2-a][1,3]diazepine dihydrochloride

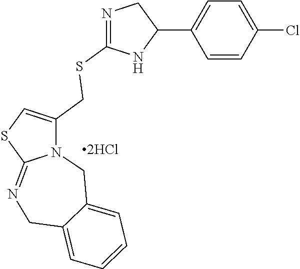 [SMILES: Clc1ccc(C2CN=C(SCC3=CSC4=NCc5ccccc5CN34)N2)cc1]

Example 22 was isolated as a beige solid (97 mg, 56%) by precipitation of the reaction mixture with Et₂O (4 mL) followed by centrifugation, trituration of the resulting solid in Et₂O (2×2 mL) and freeze-drying in water.

¹H-NMR (DMSO-d₆, 400 MHz) δ: 3.70 (dd, J11.2, 8.3 Hz, 1H, N—CHₐH_{b}); 4.31 (t, J11.2 Hz, 1H, N—CH—Ar); 4.87-4.94 (m, 3H, S—CHₐH_{b}+N-CH₂—Ar); 5.06 (d, J 15.4 Hz, 1H, S—CHₐH_{b}); 5.42 (dd, J 11.2, 8.3 Hz, 1H, N—CHₐH_{b}); 5.57 (s, 2H, N—CH₂—Ar); 7.23 (s, 1H, S—CH); 7.36-7.51 (m, 7H, 7 Ar); 7.70-7.72 (m, 1H, Ar); 11.00-11.40 (m, 3H, 2 HCl salts+NH). M/Z (M[³⁵Cl]+H)⁺: 427.0. Mp: 243-246° C.

### Example 23: 3-(((5-fluoro-1,4-dihydroquinazolin-2-yl)thio)methyl)-5,10-dihydrobenzo[e]thiazolo[3,2-a][1,3]diazepine dihydrochloride

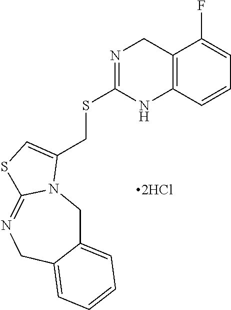 [SMILES: Fc1cccc2c1CN=C(SCC1=CSC3=NCc4ccccc4CN13)N2]

Example 23 was isolated as a beige solid (119 mg, 73%) by centrifugation of the reaction mixture followed by trituration of the solid in EtOH (2×2 mL), in MeCN (2×2 mL), in Et₂O (2×2 mL) and freeze-drying in water.

¹H-NMR (DMSO-d₆+D₂O, 400 MHz) δ: 4.67 (s, 2H, S—CH₂); 4.75 (s, 2H, N—CH₂—Ar); 4.85 (s, 2H, N—CH₂—Ar); 5.51 (s, 2H, N—CH₂—Ar); 6.92 (d, J 8.2 Hz, 1H, Ar); 7.01-7.07 (m, 2H, S-CH+Ar); 7.31-7.36 (m, 1H, Ar); 7.42-7.47 (m, 3H, 3 Ar); 7.63-7.65 (m, 1H, Ar). M/Z (M+H)⁺: 397.1. Mp>240° C.

### Example 24: 3-((((4S,5S)-4,5-diphenyl-4,5-dihydro-1H-imidazol-2-yl)thio)methyl)-5,10-dihydrobenzo[e]thiazolo[3,2-a][1,3]diazepine dihydrochloride

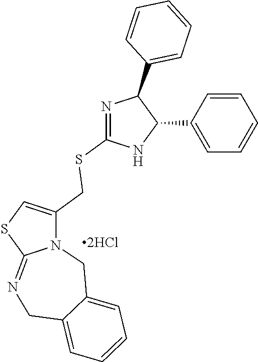 [SMILES: C1=C(CSC2=N[C@@H](c3ccccc3)[C@H](c3ccccc3)N2)N2Cc3ccccc3CN=C2S1]

Example 24 was isolated as a white solid (65 mg, 34%) by precipitation of the reaction mixture with Et₂O (4 mL) followed by centrifugation and trituration of the resulting solid in Et₂O (2×2 mL), followed by purification by preparative HPLC (column A, H₂O+0.1% HCOOH/MeCN+0.1% HCOOH 95:5 to 55:45) and freeze-drying with 1 N aqueous HCl (2 equiv).

¹H-NMR (DMSO-d₆, 400 MHz) δ: 4.88-4.95 (m, 3H, S—CH₂)+N—CH—Ar); 5.07-5.22 (m, 3H, N—CH₂—Ar+N—CH—Ar); 5.61 (s, 2H, N—CH₂—Ar); 7.22 (bs, 1H, S—CH); 7.31-7.33 (m, 4H, 4 Ar); 7.40-7.51 (m, 9H, 9 Ar); 7.71-7.74 (m, 1H, Ar); 11.38-11.47 (m, 3H, 2 HCl salts+NH). M/Z (M+H)⁺: 469.1. Mp: 178-185° C.

### Example 25: 3-(((4,5-dihydro-1H-benzo[d][1,3]diazepin-2-yl)thio)methyl)-5,10-dihydrobenzo[e]thiazolo[3,2-a][1,3]diazepine dihydrochloride

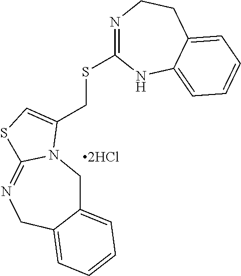 [SMILES: C1=C(CSC2=NCCc3ccccc3N2)N2Cc3ccccc3CN=C2S1]

Example 25 was isolated as an off-white solid (140 mg, 86%) by centrifugation of the reaction mixture followed by trituration of the solid in EtOH (2×2 mL), in MeCN (2×2 mL) and in Et₂O (2×2 mL).

¹H-NMR (DMSO-d₆, 400 MHz) δ: 3.11 (bs, 2H, CH₂—Ar); 3.68 (bs, 2H, N—CH₂); 4.92 (s, 2H, S—CH₂); 4.97 (s, 2H, N—CH₂—Ar); 5.73 (s, 2H, N—CH₂—Ar); 7.00 (s, 1H, S—CH); 7.20-7.35 (m, 3H, 3 Ar); 7.45-7.47 (m, 3H, 3 Ar); 7.57-7.60 (m, 1H, Ar); 7.78 (bs, 1H, Ar); 11.18 (bs, 1H, HCl salt); 11.39 (bs, 1H, HCl salt); 12.07 (bs, 1H, NH). M/Z (M+H)⁺: 393.1. Mp>250° C.

### Example 26: 3-(((4-cyclohexyl-4,5-dihydro-1H-imidazol-2-yl)thio)methyl)-5,10-dihydrobenzo[e]thiazolo[3,2-a][1,3]diazepine dihydrochloride

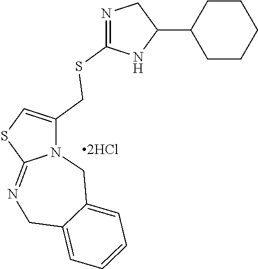 [SMILES: C1=C(CSC2=NCC(C3CCCCC3)N2)N2Cc3ccccc3CN=C2S1]

Example 26 was isolated as a white solid (45 mg, 27%) by centrifugation of the reaction mixture followed by recrystallization of the solid from EtOH (1 mL), trituration in MeCN (2×2 mL), in Et₂O (2×2 mL) and freeze-drying in water.

¹H-NMR (DMSO-d₆, 400 MHz) δ: 0.74-0.83 (m, 1H, CHₐH_{b}); 0.91-1.00 (m, 1H, CHₐH_{b}); 1.05-1.20 (m, 3H, CH₂+CH); 1.38-1.44 (m, 2H, CH₂); 1.58-1.68 (m, 4H, 2 CH₂); 3.63 (dd, J 11.2, 7.3 Hz, 1H, N—CHₐH_{b}); 3.87 (t, J7.3 Hz, 1H, N—CHₐH_{b}); 4.03-4.09 (m, 1H, N—CH); 4.87-4.91 (m, 3H, S—CHₐH_{b}+N-CH₂—Ar); 5.05 (d, J 15.4 Hz, 1H, S—CHₐH_{b}); 5.56 (s, 2H, N—CH₂—Ar); 7.25 (s, 1H, S—CH); 7.43-7.49 (m, 3H, 3 Ar); 7.70-7.73 (m, 1H, Ar); 10.80 (bs, 2H, 2 HCl salts); 11.23 (bs, 1H, NH). M/Z (M+H)⁺: 399.2. Mp: 174-180° C.

### Example 27: 3-(((4-phenyl-3,4-dihydroquinazolin-2-yl)thio)methyl)-5,10-dihydrobenzo[e]thiazolo[3,2-a][1,3]diazepine dihydrochloride

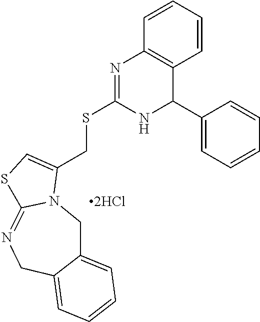 [SMILES: C1=C(CSC2=Nc3ccccc3C(c3ccccc3)N2)N2Cc3ccccc3CN=C2S1]

Example 27 was isolated as a white solid (150 mg, 82%) by centrifugation of the reaction mixture followed by trituration of the solid in EtOH (2×2 mL), in MeCN (2×2 mL) and in Et₂O (2×2 mL).

¹H-NMR (DMSO-d₆+D₂O, 400 MHz) δ: 4.66 (d, J 15.7 Hz, 1H, S—CHₐH_{b}); 4.79-4.90 (m, 3H, S—CHₐH_{b}+N-CH₂—Ar); 5.50 (s, 2H, N—CH₂—Ar); 6.01 (s, 1H, N—CH—Ar); 6.77 (s, 1H, S—CH); 7.04 (d, J7.0 Hz, 1H, Ar); 7.16-7.23 (m, 4H, 4 Ar); 7.28-7.36 (m, 4H, 4 Ar); 7.41-7.46 (m, 3H, 3 Ar); 7.63 (d, J7.0 Hz, 1H, Ar). M/Z (M+H)⁺: 455.1. Mp: 203-210° C.

### Example 28: 3-(((3,4-dihydroquinazolin-2-yl)thio)methyl)-5H-pyrido[2,3-d]thiazolo[3,2-a]pyrimidine trihydrochloride

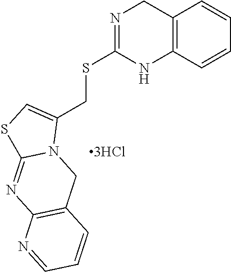 [SMILES: C1=C(CSC2=NCc3ccccc3N2)N2Cc3cccnc3N=C2S1]

Example 28 was isolated as a white solid (50 mg, 58%) by centrifugation of the reaction mixture followed by trituration of the solid in MeCN (2×2 mL), in Et₂O (2×2 mL) and freeze-drying in water.

¹H-NMR (DMSO-d₆, 400 MHz) δ: 4.68 (s, 2H, S—CH₂); 4.98 (s, 2H, N—CH₂—Ar); 5.68 (s, 2H, N—CH₂—Ar); 7.19-7.27 (m, 3H, 3 Ar); 7.30-7.36 (m, 3H, S-CH+2 Ar); 7.81 (d, J 6.9 Hz, 1H, Ar); 8.16 (d, J 4.4 Hz, 1H, Ar); 11.31 (bs, 1H, HCl salt); 12.99 (bs, 2H, HCl salt+NH). HCl salt signal not observed. M/Z (M+H)⁺: 366.1. Mp: 180-190° C.

### Example 29: 3-(((5-butyl-4,5-dihydro-1H-imidazol-2-yl)thio)methyl)-5H-thiazolo[2,3-b]quinazoline dihydrochloride

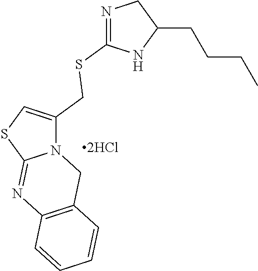 [SMILES: CCCCC1CN=C(SCC2=CSC3=Nc4ccccc4CN23)N1]

Example 29 was isolated as a white solid (130 mg, 82%) by centrifugation of the reaction mixture followed by trituration of the solid in MeCN (2×2 mL) and in Et₂O (2×2 mL).

¹H-NMR (DMSO-d₆, 400 MHz) δ: 0.88 (t, J7.0 Hz, 3H, CH₃); 1.20-1.35 (m, 4H, 2 CH₂); 1;50-1.65 (m, 2H, CH₂); 3.52 (dd, J 11.0, 7.5 Hz, 1H, N—CHₐH_{b}); 3.96 (t, J 11.0 Hz, 1H, N—CHₐH_{b}); 4.21-4.29 (m, 1H, N—CH); 4.83 (d, J 15.8 Hz, 1H, S—CHₐH_{b}); 4.98 (d, J 15.8 Hz, 1H, S—CHₐH_{b}); 5.50 (d, J 14.8 Hz, 1H, N—CHₐH_{b}—Ar); 5.55 (d, J 14.8 Hz, 1H, N—CHₐH_{b}—Ar); 7.09 (d, J7.5 Hz, 1H, Ar); 7.20-7.27 (m, 2H, 2 Ar); 7.34-7.39 (m, 1H, Ar); 7.44 (s, 1H, S—CH); 10.80 (bs, 1H, HCl salt); 11.03 (bs, 1H, HCl salt); 13.58 (bs, 1H, NH). M/Z (M+H)⁺: 359.1. Mp: 227-234° C.

### Example 30: 3-(((5-methyl-5-phenyl-4,5-dihydro-1H-imidazol-2-yl)thio)methyl)-5,10-dihydrobenzo[e]thiazolo[3,2-a][1,3]diazepine dihydrochloride

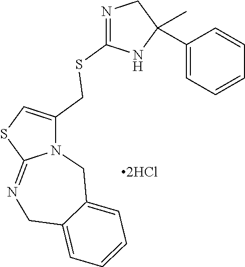 [SMILES: CC1(c2ccccc2)CN=C(SCC2=CSC3=NCc4ccccc4CN23)N1]

Example 30 was isolated as a beige solid (118 mg, 71%) by centrifugation of the reaction mixture followed by trituration of the solid in MeCN (2×2 mL) and in Et₂O (2×2 mL).

¹H-NMR (DMSO-d₆, 400 MHz) δ: 1.71 (s, 3H, CH₃); 3.90 (d, J 11.5 Hz, 1H, N—CHₐH_{b}); 4.04 (d, J 11.5 Hz, 1H, N—CHₐH_{b}); 4.86-4.94 (m, 2H, N—CH₂—Ar); 5.00 (d, J 15.5 Hz, 1H, S—CHₐH_{b}); 5.10 (d, J 15.5 Hz, 1H, S—CHₐH_{b}); 5.54-5.62 (m, 2H, N—CH₂—Ar); 7.30 (s, 1H, S—CH); 7.32-7.36 (m, 1H, Ar); 7.39-7.49 (m, 7H, 7 Ar); 7.72-7.74 (m, 1H, Ar); 11.07 (bs, 1H, HCl salt); 11.31 (bs, 1H, HCl salt); 11.65 (bs, 1H, NH). M/Z (M+H)⁺: 407.1. Mp: 195-200° C.

### Example 31: 3-(((1,4-dihydropyrido[2,3-d]pyrimidin-2-yl)thio)methyl)-5,10-dihydrobenzo[e]thiazolo[3,2-a][1,3]diazepine trihydrochloride

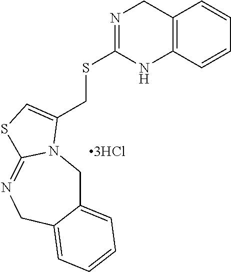 [SMILES: C1=C(CSC2=NCc3ccccc3N2)N2Cc3ccccc3CN=C2S1]

Example 31 was isolated as a beige solid (45 mg, 53%) by centrifugation of the reaction mixture followed by trituration of the solid in MeCN (2×2 mL), in Et₂O (2×2 mL) and freeze-drying in water.

¹H-NMR (DMSO-d₆, 400 MHz) δ: 4.71-4.74 (m, 4H, N—CH₂—Ar+S-CH₂); 4.89 (m, 2H, N—CH₂—Ar); 5.57 (s, 2H, N—CH₂—Ar); 7.27-7.30 (m, 2H, 2 Ar); 7.43-7.49 (m, 3H, 2 Ar+S-CH); 7.68-7.69 (m, 1H, Ar); 7.85 (bs, 1H, Ar); 8.18-8.19 (m, 1H, Ar); 10.04 (bs, 1H, HCl salt); 11.23 (bs, 1H, NH). 2 HCl salt signals not observed. M/Z (M+H)⁺: 380.1. Mp: 194-203° C.

### Example 32: 3-((((3aR,7aR)-3a,4,5,6,7,7a-hexahydro-1H-benzo[d]imidazol-2-yl)thio)methyl)-5H-thiazolo[2,3-b]quinazoline dihydrochloride

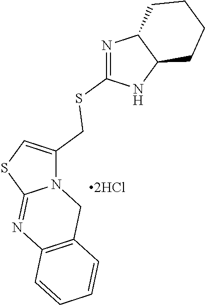 [SMILES: C1=C(CSC2=N[C@@H]3CCCC[C@H]3N2)N2Cc3ccccc3N=C2S1]

Example 32 was isolated as a white solid (143 mg, 91%) by centrifugation of the reaction mixture followed by trituration of the solid in MeCN (2×2 mL), and in Et₂O (2×2 mL).

¹H-NMR (DMSO-d₆+D₂O, 400 MHz) δ: 1.24-1.35 (m, 2H, CH₂); 1.47-1.55 (m, 2H, CH₂); 1.73-1.81 (m, 2H, CH₂); 2.12-2.15 (m, 2H, CH₂); 3.45-3.48 (m, 2H, 2 N—CH); 4.73 (d, J 16.0 Hz, 1H, S—CHₐH_{b}); 4.78 (d, J 16.0 Hz, 1H, S—CHₐH_{b}); 5.48 (s, 2H, N—CH₂—Ar); 7.05 (d, J 8.0 Hz, 1H, Ar); 7.21-7.29 (m, 2H, 2 Ar); 7.34-7.38 (m, 2H, 2 Ar+S-CH). M/Z (M+H)⁺: 357.1. Mp>250° C.

### Example 33: 3-(((5-benzyl-4,5-dihydro-1H-imidazol-2-yl)thio)methyl)-5H-thiazolo[2,3-b]quinazoline dihydrochloride

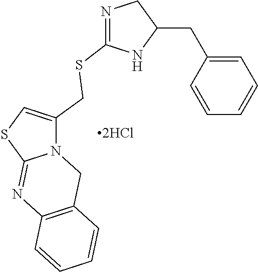 [SMILES: C1=C(CSC2=NCC(Cc3ccccc3)N2)N2Cc3ccccc3N=C2S1]

Example 33 was isolated as a white solid (159 mg, 93%) by centrifugation of the reaction mixture followed by trituration of the solid in MeCN (2×2 mL), and in Et₂O (2×2 mL).

¹H-NMR (DMSO-d₆, 400 MHz) δ: 2.92 (dd, J 13.8, 6.9 Hz, 1H, CHₐH_{b}—Ar); 2.94 (dd, J 13.8, 5.4 Hz, 1H, CHₐH_{b}—Ar); 3.59 (dd, J11.0, 6.7 Hz, 1H, N—CHₐH_{b}); 3.89 (t, J11.0 Hz, 1H, N—CHₐH_{b}); 4.57-4.64 (m, 1H, N—CH); 4.83 (d, J16.0 Hz, 1H, S—CHₐH_{b}); 4.88 (d, J16.0 Hz, 1H, S—CHₐH_{b}); 5.51 (s, 2H, N—CH₂—Ar); 7.10 (d, J7.8 Hz, 1H, Ar); 7.21-7.37 (m, 8H, 8 Ar); 7.39 (s, 1H, S—CH); 10.72 (bs, 1H, HCl salt); 11.07 (bs, 1H, HCl salt); 13.58 (bs, 1H, NH). M/Z (M+H)⁺: 393.1.Mp>250° C.

### Example 34: 3-(((5-(4-methoxybenzyl)-5-methyl-4,5-dihydro-1H-imidazol-2-yl)thio)methyl)-5,10-dihydrobenzo[e]thiazolo[3,2-a][1,3]diazepine dihydrochloride

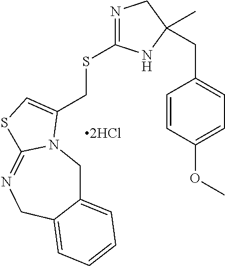 [SMILES: COc1ccc(CC2(C)CN=C(SCC3=CSC4=NCc5ccccc5CN34)N2)cc1]

Example 34 was isolated as a beige solid (150 mg, 82%) by centrifugation of the reaction mixture followed by trituration of the solid in EtOH (2×2 mL), in MeCN (2×2 mL), in Et₂O (2×2 mL) and freeze-drying in water.

¹H-NMR (DMSO-d₆, 400 MHz) δ: 1.43 (s, 3H, CH₃); 2.78 (d, J 13.9 Hz, 1H, CHₐH_{b}); 2.90 (d, J 13.9 Hz, 1H, CHₐH_{b}); 3.50 (d, J 11.4 Hz, 1H, N—CHₐH_{b}); 3.69-3.72 (m, 4H, O-CH₃+N-CHₐH_{b}); 4.81 (t, J 15.8 Hz, 2H, S—CH₂); 4.99 (m, 2H, N—CH₂—Ar); 5.45 (d, J15.2 Hz, 1H, N—CHₐH_{b}—Ar); 5.57 (d, J15.2 Hz, N—CHₐH_{b}—Ar); 6.70-6.72 (m, 2H, 2 Ar); 7.15-7.18 (m, 2H, 2 Ar); 7.21 (s, 1H, S—CH); 7.44-7.50 (m, 3H, 3 Ar); 7.67-7.69 (m, 1H, Ar); 10.39 (bs, 1H, HCl salt); 11.18 (bs, 1H, HCl salt); 11.34 (bs, 1H, NH). M/Z (M+H)⁺: 451.2. Mp: 173-180° C.

### Example 35: 3-(((3,4-dihydroquinazolin-2-yl)thio)methyl)-5,6-dihydrobenzo[d]thiazolo[3,2-a][1,3]diazepine dihydrochloride

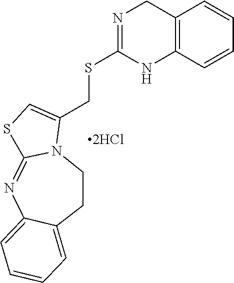 [SMILES: C1=C(CSC2=NCc3ccccc3N2)N2CCc3ccccc3N=C2S1]

Example 35 was isolated as a white solid (140 mg, 89%) by centrifugation of the reaction mixture followed by trituration of the solid in MeCN (2×2 mL) and in Et₂O (2×2 mL).

¹H-NMR (DMSO-d₆+D₂O, 400 MHz) δ: 3.33-3.34 (m, 2H, CH₂—Ar); 4.39-4.41 (m, 2H, N—CH₂); 4.66 (s, 2H, S—CH₂); 4.72 (s, 2H, N—CH₂—Ar); 7.09-7.11 (m, 1H, Ar); 7.14-7.27 (m, 5H, 4 Ar+S-CH); 7.30-7.35 (m, 3H, 3 Ar). M/Z (M+H)⁺: 379.1. Mp: 248-249° C.

### Example 36: 3-(((1-methyl-4,5-dihydro-1H-imidazol-2-yl)thio)methyl)-5H-thiazolo[2,3-b]quinazoline dihydrochloride

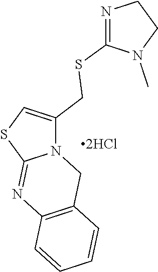 [SMILES: CN1CCN=C1SCC1=CSC2=Nc3ccccc3CN12]

Example 36 was isolated as a white solid (100 mg, 70%) by centrifugation of the reaction mixture followed by trituration of the solid in MeCN (2×2 mL) and in Et₂O (2×2 mL).

¹H-NMR (DMSO-d₆, 400 MHz) δ: 3.04 (s, 3H, CH₃); 3.80-3.86 (m, 2H, N—CH₂); 3.88-3.94 (m, 2H, N—CH₂); 4.97 (s, 2H, S—CH₂); 5.55 (s, 2H, N—CH₂—Ar); 7.10 (d, J8.2 Hz, 1H, Ar); 7.20-7.27 (m, 2H, 2 Ar); 7.34-7.39 (m, 1H, Ar); 7.50 (s, 1H, S—CH); 11.06 (bs, 1H, HCl salt); 13.65 (bs, 1H, HCl salt). M/Z (M+H)⁺: 317.1. Mp: 247-248° C.

### Example 37: 3-(((1-butyl-4,5-dihydro-1H-imidazol-2-yl)thio)methyl)-5,10-dihydrobenzo[e]thiazolo[3,2-a][1,3]diazepine dihydrochloride

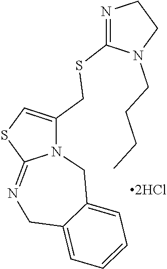 [SMILES: CCCCN1CCN=C1SCC1=CSC2=NCc3ccccc3CN12]

Example 37 was isolated as a white solid (50 mg, 32%) by centrifugation of the reaction mixture followed by trituration of the solid in MeCN (2×2 mL), in Et₂O (2×2 mL) and freeze-drying in water.

¹H-NMR (DMSO-d₆, 400 MHz) δ: 0.73 (t, J7.5 Hz, 3H, CH₃); 0.84-0.93 (m, 2H, CH₂); 1.29-1.36 (m, 2H, CH₂); 3.26 (t, J 6.9 Hz, 2H, N—CH₂); 3.84 (s, 4H, 2 N—CH₂); 4.89 (s, 2H, S—CH₂); 5.03 (s, 2H, N—CH₂—Ar); 5.58 (s, 2H, N—CH₂—Ar); 7.19 (s, 1H, S—CH); 7.43-7.49 (m, 3H, 3 Ar); 7.70-7.73 (m, 1H, Ar); 11.21 (bs, 1H, HCl salt); 11.36 (bs, 1H, HCl salt). M/Z (M+H)⁺: 373.1. Mp: 206-212° C.

### Example 38: 3-(((3,4-dihydroquinazolin-2-yl)thio)methyl)-6-methyl-6-phenyl-5,6-dihydroimidazo[2,1-b]thiazole dihydrochloride

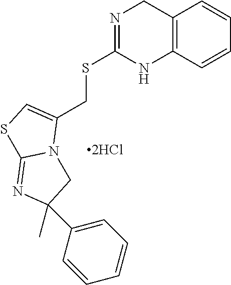 [SMILES: CC1(c2ccccc2)CN2C(CSC3=NCc4ccccc4N3)=CSC2=N1]

Example 38 was isolated as a white solid (60 mg, 39%) by centrifugation of the reaction mixture followed by trituration of the solid in MeCN (2×2 mL) and in Et₂O (2×2 mL).

¹H-NMR (DMSO-d₆, 400 MHz) δ: 1.87 (s, 3H, CH₃); 4.57 (d, J10.9 Hz, 1H, N—CHₐH_{b}); 4.66 (s, 2H, S—CH₂); 4.75 (d, J 10.9 Hz, 1H, N—CHₐH_{b}); 4.90 (bs, 2H, N—CH₂—Ar); 7.14 (s, 1H, S—CH); 7.22-7.34 (m, 4H, 4 Ar); 7.37-7.41 (m, 1H, Ar); 7.45-7.53 (m, 4H, 4 Ar); 11.25 (bs, 2H, 2 HCl salts); 12.87 (bs, 1H, NH). M/Z (M+H)⁺: 393.1. Mp: 230-235° C.

### Example 39: 3-(((6-chloro-1,4-dihydroquinazolin-2-yl)thio)methyl)-5H-thiazolo[2,3-b]quinazoline dihydrochloride

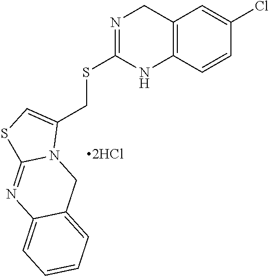 [SMILES: Clc1ccc2c(c1)CN=C(SCC1=CSC3=Nc4ccccc4CN13)N2]

Example 39 was isolated as a white solid (140 mg, 81%) by centrifugation of the reaction mixture followed by trituration of the solid in EtOH (2×2 mL), in MeCN (2×2 mL), in Et₂O (2×2 mL) and freeze-drying in water.

¹H-NMR (DMSO-d₆, 400 MHz) δ: 4.65 (s, 2H, S—CH₂); 4.97 (bs, 2H, N—CH₂—Ar); 5.57 (s, 2H, N—CH₂—Ar); 7.09 (d, J7.8 Hz, 1H, Ar); 7.21-7.27 (m, 3H, 3 Ar); 7.35-7.41 (m, 4H, 3 Ar+S-CH); 11.30 (bs, 1H, HCl salt); 13.04 (bs, 1H, HCl salt); 13.39 (bs, 1H, NH). M/Z (M[³⁵Cl]+H)⁺: 399.1. Mp: 193-200° C.

### Example 40: 7-chloro-3-((((3aR,7aR)-3a,4,5,6,7,7a-hexahydro-1H-benzo[d]imidazol-2-yl)thio)methyl)-5H-thiazolo[2,3-b]quinazoline dihydrochloride

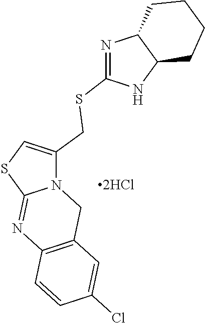 [SMILES: Clc1ccc2c(c1)CN1C(CSC3=N[C@@H]4CCCC[C@H]4N3)=CSC1=N2]

Example 40 was isolated as a white solid (115 mg, 76%) by centrifugation of the reaction mixture followed by trituration of the solid in MeCN (3 mL), in Et₂O (3 mL) and freeze-drying in water (10 mL).

¹H-NMR (DMSO-d₆, 400 MHz) δ: 1.26-1.37 (m, 2H, CH₂); 1.46-1.57 (m, 2H, CH₂); 1.72-1.83 (m, 2H, CH₂); 2.15 (d, J11.1 Hz, 2H, CH₂); 3.43-3.49 (m, 2H, 2 N—CH); 4.85 (d, J15.8 Hz, 1H, S—CHₐH_{b}); 4.93 (d, J15.8 Hz, 1H, S—CHₐH_{b}); 5.51 (s, 2H, N—CH₂); 7.11 (d, J 8.5 Hz, 1H, Ar); 7.35 (d, J 2.2 Hz, 1H, Ar); 7.42 (dd, J 8.5, 2.2 Hz, 1H, Ar); 7.48 (s, 1H, S—CH); 11.28 (bs, 2H, NH+HCl salt); 13.82 (bs, 1H, HCl salt). M/Z (M[³⁵Cl]+H)⁺: 391.0. Mp: 195-200° C.

### Example 41: 3-(((5-butyl-4,5-dihydro-1H-imidazol-2-yl)thio)methyl)-7-chloro-5H-thiazolo[2,3-b]quinazoline dihydrochloride

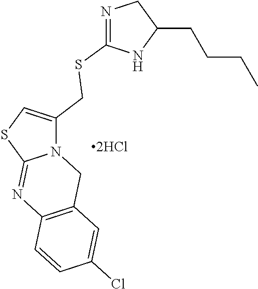 [SMILES: CCCCC1CN=C(SCC2=CSC3=Nc4ccc(Cl)cc4CN23)N1]

Example 41 was isolated as a white solid (117 mg, 77%) by centrifugation of the reaction mixture followed by trituration of the solid in MeCN (3×2 mL), in Et₂O (3 mL), in EtOH (2×2 mL) and freeze-drying in water (10 mL).

¹H-NMR (DMSO-d₆, 400 MHz) δ: 0.88 (t, J7.0 Hz, 3H, CH₃); 1.19-1.35 (m, 4H, 2 CH₂); 1.49-1.66 (m, 2H, CH₂); 3.52 (dd, J 11.0, 7.6 Hz, 1H, N—CHₐH_{b}); 3.96 (t, J 11.0 Hz, 1H, N—CHₐH_{b}); 4.21-4.29 (m, 1H, N—CH); 4.79-4.84 (m, 1H, S—CHₐH_{b}); 4.93-4.98 (m, 1H, S—CHₐH_{b}); 5.47-5.46 (m, 2H, N—CH₂); 7.11 (d, J8.5 Hz, 1H, Ar); 7.35 (d, J2.2 Hz, 1H, Ar); 7.40-7.44 (m, 2H, Ar+S-CH); 10.79 (m, 1H, NH); 11.02 (m, 1H, HCl salt); 13.83 (bs, 1H, HCl salt). M/Z (M[³⁵Cl]+H)⁺: 393.1. Mp: 234-240° C.

### Example 42: 8-chloro-3-(((1,4-dihydroquinazolin-2-yl)thio)methyl)-5H-thiazolo[2,3-b]quinazoline dihydrochloride

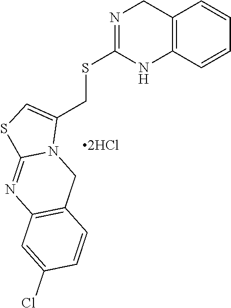 [SMILES: Clc1ccc2c(c1)N=C1SC=C(CSC3=NCc4ccccc4N3)N1C2]

Example 42 was isolated as a white solid (130 mg, 85%) by centrifugation of the reaction mixture followed by trituration of the solid in MeCN (2×2 mL) and in Et₂O (2×2 mL).

¹H-NMR (DMSO-d₆+D₂O, 400 MHz) δ: 4.69 (s, 2H, S—CH₂); 4.73 (s, 2H, N—CH₂—Ar); 5.49 (s, 2H, N—CH₂—Ar); 7.07 (d, J1.9 Hz, 1H, Ar); 7.11 (d, J7.3 Hz, 1H, Ar); 7.20-7.25 (m, 2H, 2 Ar): 7.27-7.34 (m, 4H, 3 Ar+S-CH). M/Z (M[³⁵Cl]+H)⁺: 399.0. Mp>250° C.

### Example 43: 3-(((3,4-dihydroquinazolin-2-yl)thio)methyl)-5-phenyl-5H-thiazolo[2,3-b]quinazoline dihydrochloride

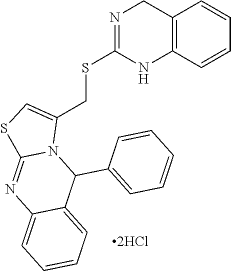 [SMILES: C1=C(CSC2=NCc3ccccc3N2)N2C(=Nc3ccccc3C2c2ccccc2)S1]

Example 43 was isolated as a white solid (90 mg, 61%) by centrifugation of the reaction mixture followed by trituration of the solid in MeCN (2×2 mL), in Et₂O (2×2 mL), in MeOH (2×2 mL) and freeze-drying in water.

¹H-NMR (DMSO-d₆, 400 MHz) δ: 4.37 (d, J15.0 Hz, 1H, N—CHₐH_{b}—Ar); 4.62-4.71 (m, 2H, S—CH₂); 4.89 (d, J15.0 Hz, 1H, N—CHₐH_{b}—Ar); 7.18-7.26 (m, 6H, 4 Ar+S-CH+N—CH—Ar); 7.29-7.43 (m, 9H, 9 Ar); 11.09 (bs, 1H, HCl salt); 12.72 (bs, 1H, HCl salt); 14.11 (bs, 1H, NH). M/Z (M+H)⁺: 441.1. Mp: 184-190° C.

### Example 44: 7-chloro-3-(((6-chloro-1,4-dihydroquinazolin-2-yl)thio)methyl)-5H-thiazolo[2,3-b]quinazoline dihydrochloride

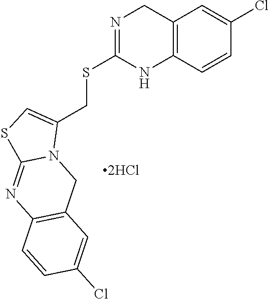 [SMILES: Clc1ccc2c(c1)CN1C(CSC3=NCc4cc(Cl)ccc4N3)=CSC1=N2]

Example 44 was isolated as a white solid (100 mg, 61%) by centrifugation of the reaction mixture followed by trituration of the solid in MeCN (2×2 mL), in Et₂O (2×2 mL), in MeOH (4×2 mL) and freeze-drying in water.

¹H-NMR (DMSO-d₆+D₂O, 400 MHz) δ: 4.57 (s, 2H, N—CH₂—Ar); 4.67 (bs, 2H, S—CH₂); 5.49 (s, 2H, N—CH₂—Ar); 7.04-7.10 (m, 2H, Ar+S-CH); 7.19-7.22 (m, 2H, 2 Ar); 7.27-7.34 (m, 2H, 2 Ar); 7.38 (dd, J 8.6, 2.3 Hz, 1H, Ar). M/Z (M[³⁵Cl]₂+H)⁺: 433.0. Mp: 242-245° C.

### Example 45: 7-chloro-3-(((1-methyl-4,5-dihydro-1H-imidazol-2-yl)thio)methyl)-5H-thiazolo[2,3-b]quinazoline dihydrochloride

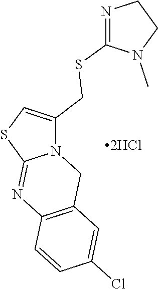 [SMILES: CN1CCN=C1SCC1=CSC2=Nc3ccc(Cl)cc3CN12]

Crude example 45 was obtained by centrifugation of the reaction mixture followed by trituration of the solid in MeCN (4×2 mL), in Et₂O (4×2 mL). The solid was then dissolved in water (10 mL) and the resulting aqueous layer was washed with DCM (2×10 mL) and freeze-dried to obtain a white solid (115 mg, 84%).

¹H-NMR (DMSO-d₆, 400 MHz) δ: 3.05 (s, 3H, N—CH₃); 3.81-3.87 (m, 2H, N—CH₂); 3.89-3.95 (m, 2H, N—CH₂); 4.88 (s, 2H, S—CH₂); 5.51 (s, 2H, N—CH₂—Ar); 7.09 (d, J8.5 Hz, 1H, Ar); 7.33 (s, 1H, Ar); 7.40-7.45 (m, 2H, Ar+S-CH); 10.84 (bs, 1H, HCl salt); 13.67 (bs, 1H, HCl salt). M/Z (M[³⁵Cl]+H)⁺: 351.0. Mp: 100-115° C.

### Example 46: 3-(((3,4-dihydroquinazolin-2-yl)thio)methyl)-6-(4-methoxybenzyl)-6-methyl-5,6-dihydroimidazo[2,1-b]thiazole dihydrochloride

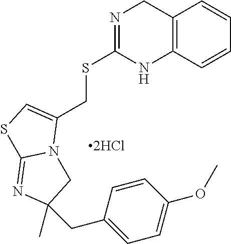 [SMILES: COc1ccc(CC2(C)CN3C(CSC4=NCc5ccccc5N4)=CSC3=N2)cc1]

Crude example 46 was obtained by concentration to dryness of the reaction mixture. The residue was then purified by preparative HPLC (column B, H₂O+0.1% HCOOH/MeCN+0.1% HCOOH 95:5 to 55:45), freeze-dried with 1 N aqueous HCl (2 equiv) and then dissolved in water (5 mL). The resulting aqueous layer was washed with DCM (2×5 mL) and freeze-dried to obtain a white solid (60 mg, 41%).

¹H-NMR (DMSO-d₆, 400 MHz) δ: 1.52 (s, 3H, CH₃); 2.96-3.03 (m, 2H, CH₂—Ar); 3.67 (s, 3H, O-CH₃); 4.21 (d, J 11.0 Hz, 1H, N—CHₐH_{b}); 4.48 (d, J 11.0 Hz, 1H, N—CHₐH_{b}); 4.65 (s, 2H, S—CH₂); 4.77-4.88 (m, 2H, N—CH₂—Ar); 6.85 (d, J8.6 Hz, 2H, 2 Ar); 6.95 (bs, 1H, S—CH); 7.24 (m, 4H, 4 Ar); 7.32 (bs, 2H, 2 Ar); 10.28 (bs, 1H, HCl salt); 11.25 (bs, 1H, HCl salt); 12.92 (bs, 1H, NH). M/Z (M+H)⁺: 437.1. Mp: 156-168° C.

### Example 47: 3-(((1-benzyl-4,5-dihydro-1H-imidazol-2-yl)thio)methyl)-7-chloro-5H-thiazolo[2,3-b]quinazoline dihydrochloride

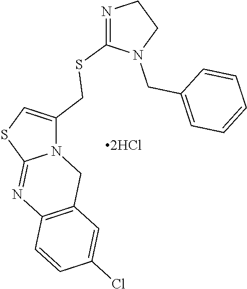 [SMILES: Clc1ccc2c(c1)CN1C(CSC3=NCCN3Cc3ccccc3)=CSC1=N2]

Crude example 47 was obtained by centrifugation of the reaction mixture followed by trituration of the solid in MeCN (2×2 mL), in Et₂O (2×2 mL). The solid was then dissolved in water (10 mL) and the resulting aqueous layer was washed with DCM (3×10 mL) and freeze-dried. The residue was purified by preparative HPLC (column A, H₂O+0.1% HCOOH/MeCN+0.1% HCOOH 95:5 to 55:45) and freeze-dried with 1 N aqueous HCl to obtain a white solid (10 mg, 12%).

¹H-NMR (DMSO-d₆, 400 MHz) δ: 3.90 (s, 4H, 2 N—CH₂); 4.67 (s, 2H, N—CH₂-Ph); 4.96 (s, 2H, S—CH₂); 5.45 (s, 2H, N—CH₂—Ar); 7.12 (d, J 8.6 Hz, 1H, Ar); 7.25-7.30 (m, 4H, 3 Ar+S-CH); 7.34-7.37 (m, 2H, 2 Ar); 7.42 (dd, J 8.6, 2.1 Hz, 1H, Ar); 7.47 (bs, 1H, Ar); 11.33 (bs, 1H, HCl salt); 13.84 (bs, 1H, HCl salt). M/Z (M[³⁵Cl]+H)⁺: 427.0. Mp: 105-118° C.

### Example 48: 7-chloro-3-(((1-isopropyl-4,5-dihydro-1H-imidazol-2-yl)thio)methyl)-5H-thiazolo[2,3-b]quinazoline dihydrochloride

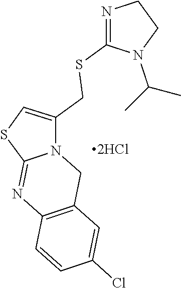 [SMILES: CC(C)N1CCN=C1SCC1=CSC2=Nc3ccc(Cl)cc3CN12]

Crude example 48 was obtained by centrifugation of the reaction mixture. The solid was dissolved in water (10 mL) and the resulting aqueous layer was washed with DCM (2×10 mL) and freeze-dried to obtain a white solid (35 mg, 48%).

¹H-NMR (DMSO-d₆, 400 MHz) δ: 1.21 (d, J6.6 Hz, 6H, 2 CH₃); 3.81-3.94 (m, 4H, 2 N—CH₂); 4.00-4.07 (m, 1H, N—CH); 4.94 (s, 2H, S—CH₂); 5.53 (s, 2H, N—CH₂—Ar); 7.10 (d, J 8.6 Hz, 1H, Ar); 7.34 (d, J 2.2 Hz, 1H, Ar); 7.42 (dd, J 8.6, 2.2 Hz, 1H, Ar); 7.44 (s, 1H, S—CH); 10.94 (bs, 1H, HCl salt); 13.80 (bs, 1H, HCl salt). M/Z (M[³⁵Cl]+H)⁺: 379.1. Mp: 150-155° C.

### Example 49: 7-chloro-3-(((1,5,6,7,8,8a-hexahydroimidazo[1,5-a]pyridin-3-yl)thio)methyl)-5H-thiazolo[2,3-b]quinazoline dihydrochloride

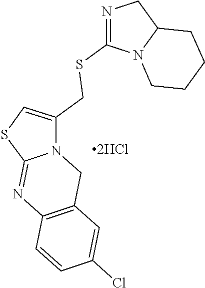 [SMILES: Clc1ccc2c(c1)CN1C(CSC3=NCC4CCCCN34)=CSC1=N2]

Crude example 49 was obtained by centrifugation of the reaction mixture. The solid was dissolved in water (10 mL) and the resulting aqueous layer was washed with DCM (2×10 mL) and freeze-dried to obtain a white solid (63 mg, 84%).

¹H-NMR (DMSO-d₆+D₂O, 400 MHz) δ: 1.36-1.50 (m, 3H, CH₂+CHₐH_{b}); 1.71-1.81 (m, 2H, CH₂); 1.91-1.94 (m, 1H, CHₐH_{b}); 3.17-3.23 (m, 1H, N—CHₐH_{b}); 3.46 (dd, J10.7, 8.8 Hz, 1H, N—CHₐH_{b}); 3.73-3.77 (m, 1H, N—CHₐH_{b}); 4.00 (t, J 10.7 Hz, 1H, N—CHₐH_{b}); 4.08-4.16 (m, 1H, N—CH); 4.67 (s, 2H, S—CH₂); 5.44 (s, 2H, N—CH₂—Ar); 7.03 (d, J8.6 Hz, 1H, Ar); 7.27 (s, 1H, S—CH); 7.36-7.41 (m, 2H, 2 Ar). M/Z (M[³⁵Cl]+H)⁺: 391.0. Mp: 150-158° C.

### Example 50: 1-(2-((5-benzyl-4,5-dihydro-1H-imidazol-2-yl)thio)ethyl)piperidine dihydrochloride

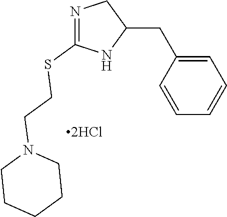 [SMILES: c1ccc(CC2CN=C(SCCN3CCCCC3)N2)cc1]

Crude example 50 was obtained by concentration to dryness of the reaction mixture. Then the residue was passed through an ISOLUTE® SCX-2 cartridge (DCM and MeOH, then NH₃ 7 M in MeOH) and purified by flash chromatography (DCM 100% to DCM/MeOH 90:10). The resulting yellow sticky oil was dissolved in water and 1 N aqueous HCl (2 equiv) and freeze-dried to afford a yellow solid (25 mg, 20%).

¹H-NMR (D₂O, 400 MHz) δ: 1.48-1.58 (m, 1H, N—CH₂—CH₂—CHₐH_{b}); 1.72-1.88 (m, 3H, N—CH₂—CH₂+N-CH₂—CH₂—CHₐH_{b}); 1.97-2.03 (m, 2H, N—CH₂—CH₂); 2.95-3.11 (m, 4H); 3.21-3.28 (m, 1H); 3.33-3.40 (m, 1H); 3.46-3.61 (m, 4H); 3.84 (dd, J 11.0, 5.8 Hz, 1H, N—CHₐH_{b}—CH); 4.05 (t, J 11.0 Hz, 1H, N—CHₐH_{b}—CH); 4.71-4.75 (m, 1H, N—CH); 7.36-7.50 (m, 5H, 5 Ar). M/Z (M+H)⁺: 304.2. Mp: 50-60° C.

### Example 51: 2-(((5-benzyl-4,5-dihydro-1H-imidazol-2-yl)thio)methyl)imidazo[1,2-a]pyrimidine hydrochloride

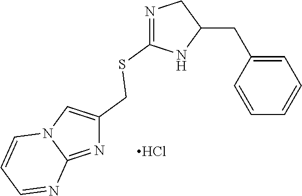 [SMILES: c1ccc(CC2CN=C(SCc3cn4cccnc4n3)N2)cc1 ; Cl]

Crude example 51 was obtained by filtration the reaction mixture followed by concentration to dryness of the filtrate. Then the residue was passed through an ISOLUTE® SCX-2 cartridge (DCM and MeOH, then NH₃ 7 M in MeOH) and purified by flash chromatography (DCM 100% to DCM/MeOH 90:10). The resulting yellow sticky solid was dissolved in water and aqueous 1 N HCl (2 equiv) and freeze-dried to obtain a yellow solid (167 mg, 60%).

¹H-NMR (DMSO-d₆, 400 MHz) δ: 2.89-2.98 (m, 2H, Ar—CH₂); 3.60 (q, J 11.0, 6.7 Hz, 1H, N—CHₐH_{b}—CH); 3.90 (t, J 11.0 Hz, 1H, N—CHₐH_{b}—CH); 4.58-4.65 (m, 1H, N—CH); 4.78 (s, 2H, S—CH₂); 7.17-7.22 (m, 1H, Ar); 7.27-7.33 (m, 5H, 5 Ar); 8.12 (s, 1H, Ar); 8.74 (dd, J 4.2, 1.9 Hz, 1H, Ar); 9.13 (dd, J6.8, 1.9 Hz, 1H, Ar); 10.54 (bs, 1H, HCl salt); 10.83 (bs, 1H, NH). M/Z (M+H)⁺: 324.1. Mp: 100-115° C.

### Example 52: 5-benzyl-2-((3-(pyrrolidin-1-yl)propyl)thio)-4,5-dihydro-1H-imidazole dihydrochloride

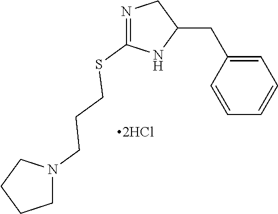 [SMILES: c1ccc(CC2CN=C(SCCCN3CCCC3)N2)cc1]

Crude example 52 was obtained by filtration the reaction mixture followed by concentration to dryness of the filtrate. Then the residue was passed through an ISOLUTE® SCX-2 cartridge (DCM and MeOH, then NH₃ 7 M in MeOH) and purified by flash chromatography (20 μm, DCM 100% to DCM/MeOH 80/20 to DCM/[MeOH+1% NH₄OH 28% aq.]80:20). The resulting yellow oil was dissolved in water and aqueous 1 N HCl (5 equiv) and freeze-dried to afford a white sticky solid (91 mg, 46%).

¹H-NMR (DMSO-d₆, 400 MHz) δ: 1.84-2.07 (m, 6H, 3 CH₂); 2.94-3.02 (m, 4H, N—CH₂+Ar—CH₂); 3.15-3.20 (m, 2H, N—CH₂); 3.32-3.36 (m, 2H, N—CH₂); 3.47-3.53 (m, 2H, S—CH₂); 3.58-3.62 (m, 1H, N—CHₐH_{b}—CH); 3.86-3.91 (t, J 10.9 Hz, 1H, N—CHₐH_{b}—CH); 4.55-4.63 (m, 1H, NH—CH); 7.25-7.37 (m, 5H, 5 Ar); 10.34 (bs, 1H, HCl salt); 10.70 (bs, 1H, HCl salt); 11.02 (bs, 1H, NH). M/Z (M+H)⁺: 304.1.

### Example 53: 5-benzyl-2-(((1-methylpyrrolidin-2-yl)methyl)thio)-4,5-dihydro-1H-imidazole dihydrochloride

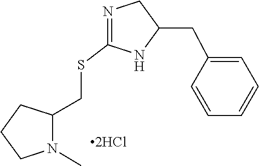 [SMILES: CN1CCCC1CSC1=NCC(Cc2ccccc2)N1 ; "·2HCl"]

Crude example 53 was obtained by filtration the reaction mixture followed by concentration to dryness of the filtrate. Then the residue was passed through an ISOLUTE® SCX-2 cartridge (DCM and MeOH, then NH₃ 3 M in MeOH) and purified twice by flash chromatography (20 μm, DCM 100% to DCM/MeOH 80/20, then 60 μm, DCM 100% to DCM/MeOH 80/20). The resulting colorless oil was dissolved in water and aqueous 1 N HCl (5 equiv) and freeze-dried to afford a yellow solid (70 mg, 37%).

¹H-NMR (DMSO-d₆, 400 MHz) δ: 1.69-1.78 (m, 1H, CHₐH_{b}); 1.87-2.07 (m, 2H, CH₂); 2.21-2.29 (m, 1H, CHₐH_{b}); 2.86- 2.87 (m, 3H, N—CH₃); 2.93-2.95 (m, 2H, S—CH₂); 3.03-3.11 (m, 1H, Ar—CHₐH_{b}); 3.42-3.48 (m, 1H, Ar—CHₐH_{b}); 3.53-3.67 (m, 3H, N—CHₐH_{b}—CH+N—CHₐH_{b}—CH₂+N—CH—CH₂); 3.81-3.87 (m, 1H, N—CHₐH_{b}—CH₂); 3.90-3.95 (t, J11.0 Hz, 1H, N—CHₐH_{b}—CH); 4.58-4.65 (m, 1H, NH—CH); 7.27-7.38 (m, 5H, 5 Ar); 10.40 (bs, 1H, HCl salt); 10.70-10.77 (m, 2H, HCl salt+NH). M/Z (M+H)⁺: 290.1. Mp: 70-80° C.

### Example 54: 5-benzyl-2-((2-(pyrrolidin-1-yl)ethyl)thio)-4,5-dihydro-1H-imidazole dihydrochloride

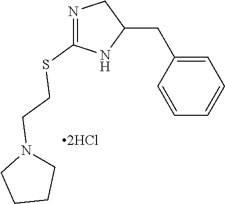 [SMILES: c1ccc(CC2CN=C(SCCN3CCCC3)N2)cc1 ; "·2HCl"]

Crude example 54 was obtained by filtration the reaction mixture followed by concentration to dryness of the filtrate. Then the residue was passed through an ISOLUTE® SCX-2 cartridge (DCM and MeOH, then NH₃ 3 M in MeOH) and purified by flash chromatography (20 μm, DCM 100% to DCM/MeOH 80/20). The resulting colorless oil was dissolved in water and aqueous 1 N HCl (5 equiv) and freeze-dried to afford a yellow solid (34 mg, 18%).

¹H-NMR (DMSO-d₆, 400 MHz) δ: 1.83-2.07 (m, 4H, 2 CH₂); 2.94-2.96 (m, 2H, Ar—CH₂); 3.01-3.11 (m, 2H, N—CH₂); 3.39-3.44 (m, 2H, N—CH₂); 3.55-3.59 (m, 4H, N—CH₂+S-CH₂); 3.62-3.66 (m, 1H, N—CHₐH_{b}—CH); 3.89-3.95 (t, J10.9 Hz, 1H, N—CHₐH_{b}—CH); 4.58-4.65 (m, 1H, NH—CH); 7.26-7.38 (m, 5H, 5 Ar); 10.32 (bs, 1H, HCl salt); 10.51 (bs, 1H, HCl salt); 10.62 (bs, 1H, NH). M/Z (M+H)⁺: 290.1.

### Example 55: 4-(3-((5-benzyl-4,5-dihydro-1H-imidazol-2-yl)thio)propyl)pyridine dihydrochloride

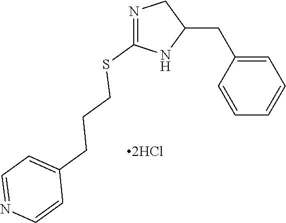 [SMILES: c1ccc(CC2CN=C(SCCCc3ccncc3)N2)cc1 ; "·2HCl"]

Crude example 55 was obtained by concentration to dryness of the reaction mixture. Then the residue was passed through an ISOLUTE® SCX-2 cartridge (DCM and MeOH, then NH₃ 3 M in MeOH) and purified by flash chromatography (20 μm, DCM 100% to DCM/MeOH 80/20). The resulting orange oil was dissolved in water and aqueous 1 N HCl (5 equiv) and freeze-dried to afford a brown solid (143 mg, 72%).

¹H-NMR (DMSO-d₆, 400 MHz) δ: 1.93-2.05 (m, 2H, CH₂); 2.93-3.00 (m, 4H, Ar—CH₂+S-CH₂); 3.26-3.30 (m, 2H, Ar—CH₂); 3.57-3.641 (m, 1H, N—CHₐH_{b}—CH); 3.85-3.90 (t, J10.9 Hz, 1H, N—CHₐH_{b}—CH); 4.56-4.60 (m, 1H, N—CH); 7.20-7.36 (m, 5H, 5 Ar); 7.90-7.93 (m, 2H, 2 Ar); 8.80-8.84 (m, 2H, 2 Ar); 10.37 (bs, 1H, HCl salt); 10.78 (bs, 1H, HCl salt); NH signal not observed. M/Z (M+H)⁺: 312.5.

### Example 56: 4-(((5-benzyl-4,5-dihydro-1H-imidazol-2-yl)thio)methyl)pyridine dihydrochloride

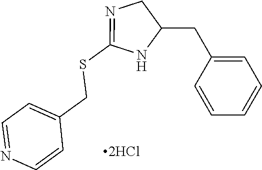 [SMILES: c1ccc(CC2CN=C(SCc3ccncc3)N2)cc1 ; "·2HCl"]

Crude example 56 was obtained by filtration the reaction mixture followed by concentration to dryness of the filtrate. Then the residue was passed through an ISOLUTE® SCX-2 cartridge (DCM and MeOH, then NH₃ 3 M in MeOH) and purified by flash chromatography (20 μm, DCM 100% to DCM/MeOH 80/20). The resulting orange oil was dissolved in water and aqueous 1 N HCl (5 equiv) and freeze-dried to give an orange solid (53 mg, 29%).

¹H-NMR (DMSO-d₆, 400 MHz) δ: 2.87-2.88 (m, 2H, Ar—CH₂); 3.53-3.57 (m, 1H, N—CHₐH_{b}—CH); 3.83-3.88 (t, J10.9 Hz, 1H, N—CHₐH_{b}—CH); 4.53-4.61 (m, 1H, N—CH); 4.88 (s, 2H, S—CH₂); 7.20-7.30 (m, 5H, 5 Ar); 7.98-8.00 (d, J6.6 Hz, 2H, 2 Ar); 8.84-8.86 (d, J 6.6 Hz, 2H, 2 Ar); 10.71 (bs, 1H, HCl salt); 11.05 (bs, 1H, HCl salt); NH signal not observed. M/Z (M+H)⁺: 284.1.

### Example 57: 5-benzyl-2-((2-(1-methylpyrrolidin-2-yl)ethyl)thio)-4,5-dihydro-1H-imidazole dihydrochloride

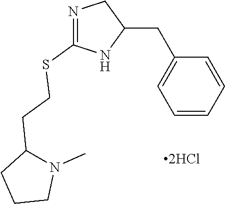 [SMILES: CN1CCCC1CCSC1=NCC(Cc2ccccc2)N1 ; "·2HCl"]

Crude example 57 was obtained by filtration the reaction mixture followed by concentration to dryness of the filtrate. Then the residue was passed through an ISOLUTE® SCX-2 cartridge (DCM and MeOH, then NH₃ 3 M in MeOH) and purified by flash chromatography (KP—NH, DCM 100% to DCM/MeOH 95/5). The resulting colorless oil was further purified by preparative HPLC (column B, H₂O+0.1% HCOOH/MeCN+0.1% HCOOH 95:5 to 55:45) and freeze-dried with 1 N aqueous HCl (5 equiv) to obtain a white sticky solid (24 mg, 8%).

¹H-NMR (DMSO-d₆, 400 MHz) δ: 1.66-1.76 (m, 1H, CHₐH_{b}); 1.88-2.04 (m, 3H, CH₂+CHₐH_{b}); 2.14-2.30 (m, 2H, CH₂); 2.77-2.79 (m, 3H, N—CH₃); 2.94-2.96 (d, J6.2 Hz, 2H, Ar—CH₂); 2.99-3.07 (m, 1H, N—CHₐH_{b}); 3.29-3.40 (m, 3H, S—CH₂+N-CHₐH_{b}); 3.48-3.56 (m, 1H, N—CH); 3.58-3.62 (m, 1H, N—CHₐH_{b}—CH); 3.84-3.87 (m, 1H, N—CHₐH_{b}—CH); 4.56-4.63 (m, 1H, NH—CH); 7.26-7.37 (m, 5H, 5 Ar); 10.39 (bs, 1H, HCl salt); 10.74 (bs, 1H, HCl salt); 10.94 (bs, 1H, NH). M/Z (M+H)⁺: 304.2.

### Example 58: 1-(2-((5-benzyl-4,5-dihydro-1H-imidazol-2-yl)thio)ethyl)azepane dihydrochloride

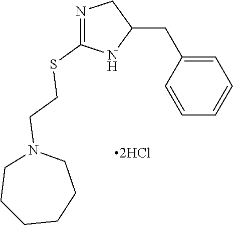 [SMILES: c1ccc(CC2CN=C(SCCN3CCCCCC3)N2)cc1 ; "·2HCl"]

Crude example 58 was obtained by concentration to dryness of the reaction mixture. Then the residue was passed through an ISOLUTE® SCX-2 cartridge (DCM and MeOH, then NH₃ 3 M in MeOH) and purified by flash chromatography (20 μm, DCM 100% to DCM/MeOH 90/10). The resulting colorless oil was dissolved in water and aqueous 1 N HCl (5 equiv) and freeze-dried to afford a yellow solid (18 mg, 13%).

¹H-NMR (DMSO-d₆, 400 MHz) δ: 1.56-1.70 (m, 4H, 2 CH₂); 1.80-1.86 (m, 4H, 2 CH₂); 2.94-2.96 (d, J6.2 Hz, 2H, Ar—CH₂); 3.12-3.19 (m, 2H, S—CH₂); 3.38-3.44 (m, 4H, 2 N—CH₂); 3.59-3.66 (m, 3H, N—CH₂+N-CHₐH_{b}—CH); 3.89-3.95 (t, J 10.9 Hz, 1H, N—CHₐH_{b}—CH); 4.58-4.66 (m, 1H, N—CH); 7.26-7.38 (m, 5H, 5 Ar); 10.31 (m, 2H, 2 HCl salt); 10.61 (bs, 1H, NH). M/Z (M+H)⁺: 318.1. Mp: 65-85° C.

### Example 59: 6-chloro-2-((2-(pyrrolidin-1-yl)ethyl)thio)-1,4-dihydroquinazoline dihydrochloride

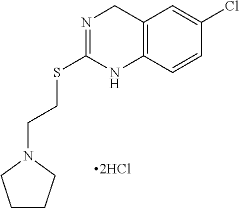 [SMILES: Clc1ccc2c(c1)CN=C(SCCN1CCCC1)N2 ; "·2HCl"]

Crude example 59 was obtained by filtration of the reaction mixture. Then the resulting white solid was passed through an ISOLUTE® SCX-2 cartridge (DCM and MeOH, then NH₃ 3 M in MeOH) and purified by flash chromatography (20 μm, DCM 100% to DCM/MeOH 90/10) and by preparative HPLC (column B, H₂O+0.1% HCOOH/MeCN+0.1% HCOOH 95:5 to 55:45) and freeze-dried with 1 N aqueous HCl (5 equiv) to obtain a white solid (63 mg, 34%).

¹H-NMR (DMSO-d₆, 400 MHz) δ: 1.89-2.01 (m, 4H, 2 CH₂); 3.01-3.26 (m, 2H, CH₂); 3.49-3.53 (m, 4H, 2 CH₂); 3.76-3.83 (m, 2H, CH₂); 4.70 (s, 2H, N—CH₂); 7.25-7.27 (m, 1H, Ar); 7.37-7.40 (m, 2H, 2 Ar); 10.99 (bs, 2H, 2 HCl salts); 12.83 (bs, 1H, NH). M/Z (M[³⁵Cl]+H)⁺: 296.0. Mp>250° C.

### Example 60: 6-chloro-2-((4-(pyrrolidin-1-yl)butyl)thio)-1,4-dihydroquinazoline dihydrochloride

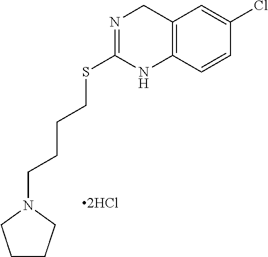 [SMILES: Clc1ccc2c(c1)CN=C(SCCCCN1CCCC1)N2 ; "·2HCl"]

Crude example 60 was obtained by filtration of the reaction mixture. Then the resulting solid was passed through an ISOLUTE® SCX-2 cartridge (DCM and MeOH, then NH₃ 3 M in MeOH) and purified by flash chromatography (20 μm, DCM 100% to DCM/MeOH 80/20), then by preparative HPLC (column B, H₂O+0.1% HCOOH/MeCN+0.1% HCOOH 95:5 to 55:45). The pure fractions containing example 60 were and freeze-dried with 1 N aqueous HCl (5 equiv) to afford a white solid (27 mg, 19%).

¹H-NMR (DMSO-d₆, 400 MHz) δ: 1.71-2.02 (m, 8H, 4 CH₂); 2.87-3.04 (m, 2H, CH₂); 3.12-3.15 (m, 2H, CH₂); 3.40-3.54 (m, 4H, 2 CH₂); 4.70 (s, 2H, N—CH₂—Ar); 7.21 (d, J8.2 Hz, 1H, Ar); 7.37-7.39 (m, 2H, 2 Ar); 10.63 (bs, 2H, 2 HCl salts); 12.57 (bs, 1H, NH). M/Z (M[³⁵Cl]+H)⁺: 324.0.

### Example 61: 2-(((5-benzyl-4,5-dihydro-1H-imidazol-2-yl)thio)methyl)-4-chlorothieno[3,2-c]pyridine dihydrochloride

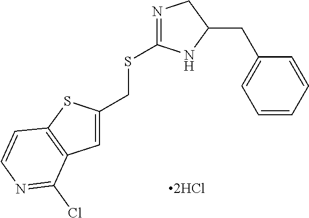 [SMILES: Clc1nccc2sc(CSC3=NCC(Cc4ccccc4)N3)cc12 ; "·2HCl"]

Crude example 61 was obtained by centrifugation of the reaction mixture followed by trituration of the solid in MeOH and a second centrifugation. Both supernatants were combined and evaporated to dryness. The residue was passed through an ISOLUTE® SCX-2 cartridge (DCM and MeOH, then NH₃ 3 M in MeOH) and then purified by flash chromatography (20 μm, DCM 100% to DCM/MeOH 90/10). Further purification of the resulting yellow solid by preparative HPLC (column A, H₂O+0.1% HCOOH/MeCN+0.1% HCOOH 90:10 to 50:50) and freeze-drying with 1 N aqueous HCl (1 mL) gave a white solid (14 mg, 9%).

¹H-NMR (DMSO-d₆, 400 MHz) δ: 2.82 (d, J 5.9 Hz, 2H, CH₂—Ar); 3.58 (dd, J11.1, 6.8 Hz, 1H, N—CHₐH_{b}); 3.90 (t, J 11.1 Hz, 1H, N—CHₐH_{b}); 4.57-4.64 (m, 1H, N—CH); 4.99 (d, J15.3 Hz, 1H, S—CHₐH_{b}); 5.06 (d, J15.3 Hz, 1H, S—CHₐH_{b}); 7.10-7.23 (m, 5H, 5 Ar); 7.70 (s, 1H, Ar); 8.12 (dd, J 5.6, 0.5 Hz, 1H, Ar); 8.28 (d, J 5.6 Hz, 1H, Ar); 10.51 (bs, 1H, HCl salt); 10.83 (bs, 1H, HCl salt); NH signal not observed. M/Z (M[³⁵Cl]+H)⁺: 374.0. Mp: 65-68° C.

### Example 62: 3-(((3,4-dihydroquinazolin-2-yl)thio)methyl)-6,7-dimethoxy-2,3-dihydrobenzo[4,5]imidazo[2,1-b]thiazol-3-ol hydrochloride

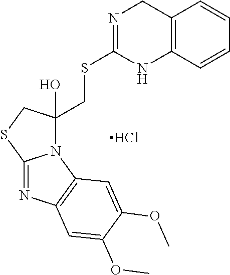 [SMILES: COc1cc2nc3n(c2cc1OC)C(O)(CSC1=NCc2ccccc2N1)CS3 ; Cl]

Example 62 was isolated as a beige solid (145 mg, quant.) by filtration of the reaction mixture and washing of the solid with MeCN.

¹H-NMR (DMSO-d₆+D₂O, 400 MHz) δ: 3.67 (d, J11.0 Hz, 1H, S—CHₐH_{b}); 3.76 (s, 6H, 2 CH₃); 3.91 (d, J14.6 Hz, 1H, S—CHₐH_{b}); 4.05 (d, J14.6 Hz, 1H, S—CHₐH_{b}); 4.08 (d, J11.0 Hz, 1H, S—CHₐH_{b}); 4.66 (d, J15.1 Hz, 1H, N—CHₐH_{b}—Ar); 4.80 (d, J 15.1 Hz, 1H, N—CHₐH_{b}—Ar); 6.86-6.89 (m, 3H, 3 Ar); 7.09-7.13 (m, 2H, 2 Ar); 7.19-7.23 (m, 1H, Ar). M/Z (M+H)⁺: 429.0. Mp: 235-240° C.

### Example 63: 3-(((3,4-dihydroquinazolin-2-yl)thio)methyl)-6-(thiophen-2-ylmethyl)-5,6-dihydroimidazo[2,1-b]thiazole dihydrochloride

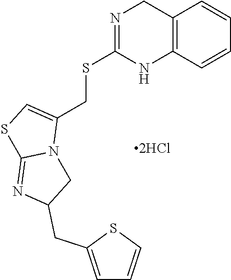 [SMILES: C1=C(CSC2=NCc3ccccc3N2)N2CC(Cc3cccs3)N=C2S1 ; "·2HCl"]

Example 63 was isolated as a white solid (92 mg, 68%) by centrifugation of the reaction mixture followed by trituration of the solid in Et₂O (2×2 mL).

¹H-NMR (DMSO-d₆, 400 MHz) δ: 3.32-3.34 (d, J6.2 Hz, 2H, N—CH—CH₂); 4.27-4.31 (m, 1H, N—CHₐH_{b}—CH); 4.56-4.61 (t, J 10.6 Hz, 1H, N—CHₐH_{b}—CH); 4.65 (s, 2H, S—CH₂); 4.77-4.93 (m, 2H, N—CH₂—Ar); 4.97-5.04 (m, 1H, N—CH); 6.99-7.05 (m, 3H, 3 Ar); 7.21-7.35 (m, 4H, 3 Ar+S-CH); 7.41-7.24 (dd, J 5.1, 1.1 Hz, 1H, Ar); 10.08 (bs, 1H, HCl salt); 11.27 (bs, 1H, HCl salt); 12.92 (bs, 1H, NH). M/Z (M+H)⁺: 399.1. Mp: 215-220° C.

### Example 64: 7-chloro-3-(((5-(thiophen-2-ylmethyl)-4,5-dihydro-1H-imidazol-2-yl)thio)methyl)-5H-thiazolo[2,3-b]quinazoline dihydrochloride

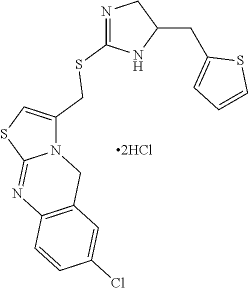 [SMILES: Clc1ccc2c(c1)CN1C(CSC3=NCC(Cc4cccs4)N3)=CSC1=N2 ; "·2HCl"]

Example 64 was isolated as a white solid (135 mg, 91%) by centrifugation of the reaction mixture followed by trituration of the solid in Et₂O (2×2 mL).

¹H-NMR (DMSO-d₆, 400 MHz) δ: 3.15-3.17 (t, J6.4 Hz, 2H, N—CH—CH₂); 3.58-3.63 (m, 1H, N—CHₐH_{b}—CH); 3.92-3.98 (t, J 11.1 Hz, 1H, N—CHₐH_{b}—CH); 4.55-4.62 (m, 1H, N—CHₐH_{b}—CH); 4.78-4.87 (m, 2H, S—CH₂); 5.50 (s, 2H, N—CH₂); 7.00-7.01 (m, 2H, 2 Ar); 7.11-7.13 (d, J8.5 Hz, 1H, Ar); 7.35-7.40 (m, 2H, Ar+S-CH); 7.41-7.44 (m, 2H, 2 Ar); 10.73 (bs, 1H, HCl salt); 11.11 (bs, 1H, HCl salt); NH signal not observed. M/Z (M[³⁵Cl]+H)⁺: 433.0. Mp>250° C.

### Example 65: 6-benzyl-3-(((3,4-dihydroquinazolin-2-yl)thio)methyl)-5,6-dihydroimidazo[2,1-b]thiazole dihydrochloride

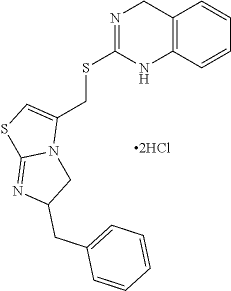 [SMILES: C1=C(CSC2=NCc3ccccc3N2)N2CC(Cc3ccccc3)N=C2S1 ; "·2HCl"]

Crude example 65 was obtained by addition of Et₂O (4 mL) to the reaction mixture followed by centrifugation and trituration of the resulting solid in Et₂O (3 mL). The solid was then dissolved in water (8 mL) and the resulting aqueous layer was washed with DCM (10 mL) and freeze-dried. Further purification by preparative HPLC (column B, H₂O+0.1% HCOOH/MeCN+0.1% HCOOH 95:5 to 55:45) and freeze-drying with 1 N aqueous HCl (3 mL) afforded a white solid (68 mg, 49%).

¹H-NMR (DMSO-d₆, 400 MHz) δ: 3.06-3.15 (m, 2H, CH₂-Ph); 4.30 (dd, J10.6, 7.0 Hz, 1H, N—CHₐH_{b}); 4.53 (t, J10.6 Hz, 1H, N—CHₐH_{b}); 4.65 (s, 2H, N—CH₂); 4.80-4.92 (m, 2H, S—CH₂); 4.99-5.07 (m, 1H, N—CH); 7.00 (s, 1H, S—CH); 7.23-7.37 (m, 9H, 9 Ar); 10.09 (bs, 1H, HCl salt); 11.34 (bs, 1H, HCl salt); 12.98 (bs, 1H, NH). M/Z (M+H)⁺: 393.0. Mp: 142-148° C.

### Example 66: 3-(((7-bromo-1,4-dihydroquinazolin-2-yl)thio)methyl)-7-chloro-5H-thiazolo[2,3-b]quinazoline dihydrochloride

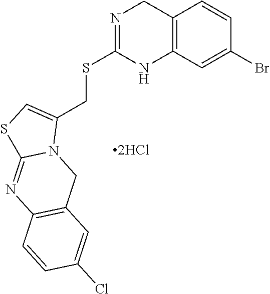 [SMILES: Clc1ccc2c(c1)CN1C(CSC3=NCc4ccc(Br)cc4N3)=CSC1=N2 ; "·2HCl"]

Example 66 was isolated as a white solid (120 mg, 67%) by centrifugation of the reaction mixture followed by trituration of the solid in MeOH (5×2 mL) and freeze-drying in water.

¹H-NMR (DMSO-d₆+D₂O, 400 MHz) δ: 4.55 (s, 4H, S—CH₂+N-CH₂—Ar); 5.47 (s, 2H, N—CH₂—Ar); 7.01 (d, J 8.4 Hz, 1H, Ar); 7.07 (d, J8.4 Hz, 1H, Ar); 7.31 (d, J1.7 Hz, 1H, Ar); 7.19 (s, 1H, S—CH); 7.32 (dd, J8.3, 1.8 Hz, 1H, Ar); 7.35 (d, J1.7 Hz, 1H, Ar); 7.40 (dd, J8.3, 1.8 Hz, 1H, Ar). M/Z (M[³⁵Cl][⁷⁹Br]+H)⁺: 479.0. Mp>250° C.

### Example 67: 3-(((6-bromo-1,4-dihydroquinazolin-2-yl)thio)methyl)-7-chloro-5H-thiazolo[2,3-b]quinazoline dihydrochloride

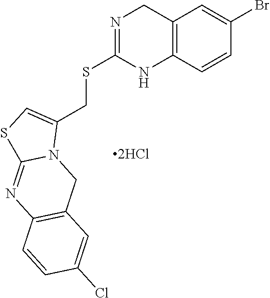 [SMILES: Clc1ccc2c(c1)CN1C(CSC3=NCc4cc(Br)ccc4N3)=CSC1=N2 ; "·2HCl"]

Example 67 was isolated as a white solid (55 mg, 31%) by centrifugation of the reaction mixture followed by trituration of the solid in MeOH (5×2 mL) and freeze-drying in water.

¹H-NMR (DMSO-d₆+D₂O, 400 MHz) δ: 4.53 (s, 2H, N—CH₂—Ar); 4.57 (s, 2H, S—CH₂); 5.44 (s, 2H, N—CH₂—Ar); 6.95 (d, J8.6 Hz, 1H, Ar); 7.00 (d, J8.6 Hz, 1H, Ar); 7.13 (s, 1H, S—CH); 7.32-7.34 (m, 2H, 2 Ar); 7.38 (dd, J8.6, 2.0 Hz, 1H, Ar); 7.42 (dd, J8.6, 2.0 Hz, 1H, Ar). M/Z (M[³⁵Cl][⁷⁹Br]+H)⁺: 479.0. Mp: 222-233° C.

### Example 68: 3-(((4,6-diazaspiro[2.4]hept-5-en-5-yl)thio)methyl)-7-chloro-5H-thiazolo[2,3-b]quinazoline dihydrochloride

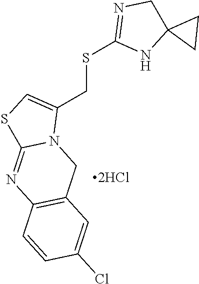 [SMILES: Clc1ccc2c(c1)CN1C(CSC3=NCC4(CC4)N3)=CSC1=N2 ; "·2HCl"]

Example 68 was isolated as a white solid (60 mg, 42%) by centrifugation of the reaction mixture followed by trituration of the solid in MeCN (2×2 mL) and in Et₂O (2×2 mL), followed by purification by preparative HPLC (column B, H₂O+0.1% HCOOH/MeCN+0.1% HCOOH 95:5 to 55:45) and freeze-drying with 1 N aqueous HCl (3 mL).

¹H-NMR (DMSO-d₆+D₂O, 400 MHz) δ: 0.91-0.95 (m, 2H, CH₂); 1.05-1.08 (m, 2H, CH₂); 3.91 (s, 2H, N—CH₂); 4.59 (s, 2H, S—CH₂); 5.41 (s, 2H, N—CH₂—Ar); 7.01 (d, J8.6 Hz, 1H, Ar); 7.24 (s, 1H, S—CH); 7.35-7.40 (m, 2H, 2 Ar). M/Z (M[³⁵Cl]+H)⁺: 363.0. Mp>250° C.

### Example 69: 7-bromo-3-(((1,4-dihydroquinazolin-2-yl)thio)methyl)-5H-thiazolo[2,3-b]quinazoline dihydrochloride

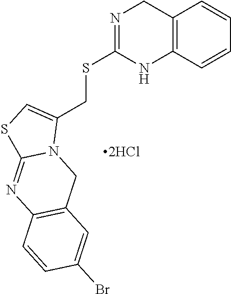 [SMILES: Brc1ccc2c(c1)CN1C(CSC3=NCc4ccccc4N3)=CSC1=N2 ; "·2HCl"]

Example 69 was isolated as a white solid (126 mg, 86%) by centrifugation of the reaction mixture followed by trituration of the solid in MeOH (3×2 mL) and in Et₂O (2×3 mL).

¹H-NMR (DMSO-d₆+D₂O, 400 MHz) δ: 4.69 (s, 2H, S—CH₂); 4.73 (s, 2H, N—CH₂); 5.50 (s, 2H, N—CH₂); 6.99 (d, J8.4 Hz, 1H, Ar); 7.12 (d, J7.8 Hz, 1H, Ar); 7.21 (d, J7.8 Hz, 1H, Ar); 7.25 (t, J7.4 Hz, 1H, Ar); 7.30 (s, 1H, S—CH); 7.33 (t, J7.4 Hz, 1H, Ar); 7.50 (d, J 2.0 Hz, 1H, Ar); 7.54 (d, J8.4, 2.0 Hz, 1H, Ar). M/Z (M[⁷⁹Br]+H)⁺: 445.0. Mp>250° C.

### Example 70: 8-bromo-3-(((1,4-dihydroquinazolin-2-yl)thio)methyl)-5H-thiazolo[2,3-b]quinazoline dihydrochloride

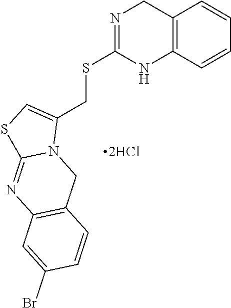 [SMILES: Brc1ccc2c(c1)N=C1SC=C(CSC3=NCc4ccccc4N3)N1C2 ; "·2HCl"]

Example 70 was isolated as a white solid (130 mg, 89%) by centrifugation of the reaction mixture followed by trituration of the solid in EtOH (3×2 mL) and in Et₂O (2×2 mL).

¹H-NMR (DMSO-d₆+D₂O, 400 MHz) δ: 4.69 (s, 2H, S—CH₂); 4.75 (s, 2H, N—CH₂); 5.47 (s, 2H, N—CH₂); 7.12 (d, J8.0 Hz, 1H, Ar); 7.20-7.27 (m, 4H, 4 Ar); 7.30-7.35 (m, 2H, S-CH+Ar); 7.40 (dd, J 8.2, 2.0 Hz, 1H, Ar). M/Z (M[⁷⁹Br]+H)⁺: 445.0. Mp>250° C.

### Example 71: 2-((2-(isoindolin-2-yl)ethyl)thio)-3,4-dihydroquinazoline dihydrochloride

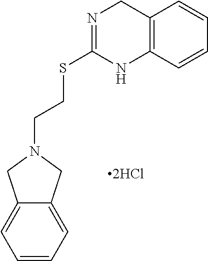 [SMILES: c1ccc2c(c1)CN(CCSC1=NCc3ccccc3N1)C2 ; "·2HCl"]

Crude example 71 was obtained by concentration to dryness of the reaction mixture followed by purification by flash chromatography (CyHex 100% to CyHex/EtOAc 0:100 then DCM 100% to DCM/MeOH 80:20). The obtained green sticky solid was dissolved in DCM (1 mL), then HCl in Et₂O (2.0 equiv) was added. The resulting suspension was concentrated to dryness and suspended in mixture of DCM and MeOH. The supernatant was removed and the solid was triturated in Et₂O (2×2 mL) to obtain a white solid (85 mg, 49%).

¹H-NMR (DMSO-d₆, 400 MHz) δ: 3.74-3.90 (m, 4H, 2 N—CH₂—Ar); 4.58-4.86 (m, 6H, N—CH₂—Ar+S-CH₂+N-CH₂); 7.16-7.34 (m, 4H, 4 Ar); 7.37-7.43 (m, 4H, 4 Ar); 10.99 (bs, 1H, HCl salt); 12.11 (bs, 1H, HCl salt); 12.65 (bs, 1H, NH). M/Z (M+H)⁺: 310.1. Mp: 134-138° C.

### Example 72: 7-chloro-3-(((5-methyl-5-phenyl-4,5-dihydro-1H-imidazol-2-yl)thio)methyl)-5H-thiazolo[2,3-b]quinazoline dihydrochloride

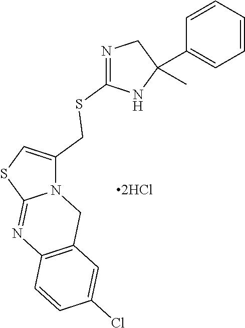 [SMILES: CC1(c2ccccc2)CN=C(SCC2=CSC3=Nc4ccc(Cl)cc4CN23)N1 ; "·2HCl"]

Example 72 was isolated as a white solid (145 mg, 89%) by centrifugation of the reaction mixture followed by trituration of the solid in EtOH (3×2 mL) and in Et₂O (2×3 mL).

¹H-NMR (DMSO-d₆, 400 MHz) δ: 1.73 (s, 3H, CH₃); 3.91 (d, J 11.4 Hz, 1H, N—CHₐH_{b}); 4.06 (d, J 11.4 Hz, 1H, N—CHₐH_{b}); 4.87 (d, J 15.8 Hz, 1H, S—CHₐH_{b}); 5.00 (d, J 15.8 Hz, 1H, S—CHₐH_{b}); 5.50 (d, J 15.0 Hz, 1H, N—CHₐH_{b}); 5.56 (d, J 15.0 Hz, 1H, N—CHₐH_{b}); 7.11 (d, J 8.6 Hz, 1H, Ar); 7.34-7.47 (m, 8H, S-CH+7 Ar); 10.99 (bs, 1H, HCl salt); 11.57 (s, 1H, HCl salt); 13.75 (bs, 1H, NH). M/Z (M[³⁵Cl]+H)⁺: 427.2. Mp>250° C.

### Example 73: 3-(((4,4-dimethyl-1,4-dihydroquinazolin-2-yl)thio)methyl)-6-fluoro-5H-thiazolo[2,3-b]quinazoline dihydrochloride

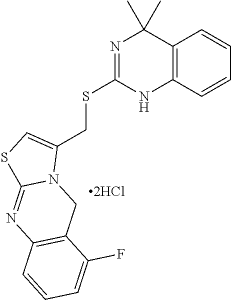 [SMILES: CC1(C)N=C(SCC2=CSC3=Nc4cccc(F)c4CN23)Nc2ccccc21 ; "·2HCl"]

Example 73 was isolated as a white solid (86 mg, 39%) by centrifugation of the reaction mixture followed by trituration of the solid in EtOH (4×2 mL), in Et₂O (2×2 mL) and freeze-drying in water (10 mL).

¹H-NMR (DMSO-d₆, 400 MHz) δ: 1.64 (s, 1H, 6H, 2 CH₃); 5.10 (bs, 2H, S—CH₂); 5.59 (s, 2H, N—CH₂); 6.92 (d, J8.0 Hz, 1H, Ar); 7.08 (t, J9.0 Hz, 1H, Ar); 7.25-7.44 (m, 6H, S-CH+5 Ar); 11.13 (bs, 1H, HCl salt); 12.89 (bs, 1H, HCl salt); 13.75 (bs, 1H, NH). M/Z (M+H)⁺: 411.1. Mp: 190-194° C.

### Example 74: 2-((2-(5-chloro-1H-indol-1-yl)ethyl)thio)-3,4-dihydroquinazoline hydrochloride

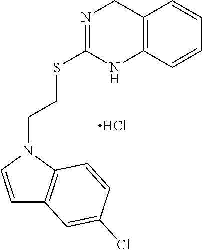 [SMILES: Clc1ccc2c(ccn2CCSC2=NCc3ccccc3N2)c1 ; Cl]

Crude example 74 was obtained by concentration to dryness of the reaction mixture. Then the residue was passed through an ISOLUTE® SCX-2 cartridge (DCM and MeOH, then NH₃ 3 M in MeOH) and concentrated to dryness. To a solution of the resulting solid in DCM was added HCl in Et₂O (2.0 equiv), and after evaporation to dryness the residue was triturated in DCM (3×2 mL) and in Et₂O (2×2 mL) to afford a yellow solid (97 mg, 66%).

¹H-NMR (DMSO-d₆, 400 MHz) δ: 3.82 (t, J6.0 Hz, 2H, N—CH₂); 4.43 (s, 2H, CH₂—Ar); 4.58 (t, J6.0 Hz, 2H, S—CH₂); 6.39 (dd, J 3.2, 0.5 Hz, 2H, 2 Ar); 6.94 (dd, J7.8, 0.9 Hz, 1H, Ar); 7.12-7.15 (m, 2H, 2 Ar); 7.17-7.29 (m, 2H, 2 Ar); 7.45 (d, J 2.0 Hz, 1H, Ar); 7.48 (d, J 3.2 Hz, 1H, Ar); 7.59 (d, J 8.7 Hz, 1H, Ar); 10.44 (bs, 1H, HCl salt); 12.07 (bs, 1H, NH). M/Z (M[³⁵Cl]+H)⁺: 342.1. Mp: 158-164° C.

### Example 75: 7-chloro-3-(((4,4-dimethyl-1,4-dihydroquinazolin-2-yl)thio)methyl)-5H-thiazolo[2,3-b]quinazoline dihydrochloride

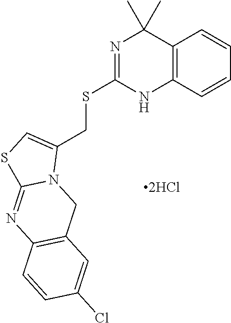 [SMILES: CC1(C)N=C(SCC2=CSC3=Nc4ccc(Cl)cc4CN23)Nc2ccccc21 ; "·2HCl"]

Example 75 was isolated as a white solid (139 mg, 86%) by centrifugation of the reaction mixture followed by trituration of the solid in EtOH (4×2 mL) in Et₂O (2×2 mL) and freeze-drying in water (10 mL).

H-NMR (DMSO-d₆, 400 MHz) δ: 1.64 (s, 6H, 2 CH₃); 5.01 (bs, 2H, S—CH₂); 5.59 (s, 2H, N—CH₂); 7.11 (d, J8.6 Hz, 1H, Ar); 7.26-7.44 (m, 7H, S-CH+6 Ar); 11.12 (bs, 1H, HCl salt); 12.92 (bs, 1H, HCl salt); 13.62 (bs, 1H, NH). M/Z (M[³⁵Cl]+H)⁺: 427.1. Mp: 192-196° C.

### Example 76: 7-chloro-3-(((4,5-dihydro-1H-benzo[d][1,3]diazepin-2-yl)thio)methyl)-5H-thiazolo[2,3-b]quinazoline dihydrochloride

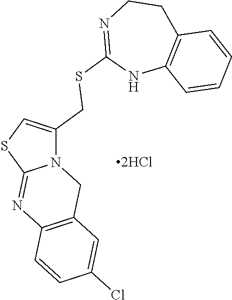 [SMILES: Clc1ccc2c(c1)CN1C(CSC3=NCCc4ccccc4N3)=CSC1=N2 ; "·2HCl"]

Example 76 was isolated as a white solid (134 mg, 85%) by centrifugation of the reaction mixture followed by trituration of the solid in EtOH (3×2 mL) in Et₂O (2×2 mL) and freeze-drying in water.

¹H-NMR (DMSO-d₆, 400 MHz) δ: 3.08-3.13 (m, 2H, CH₂-Ph); 3.63-3.70 (m, 2H, N—CH₂); 4.84 (s, 2H, S—CH₂); 5.61 (s, 2H, N—CH₂); 7.12 (d, J8.6 Hz, 1H, Ar); 7.19-7.23 (m, 1H, Ar); 7.28-7.34 (m, 4H, S-CH+3 Ar); 7.43 (dd, J8.6, 2.4 Hz, 1H, Ar); 7.48 (d, J8.0 Hz, 1H, Ar); 11.28 (bs, 1H, HCl salt); 11.97 (bs, 1H, HCl salt); 13.75 (bs, 1H, NH). M/Z (M[³⁵Cl]+H)⁺: 413.1. Mp: 215-217° C.

### Example 77: 2-((2-(pyrrolidin-1-yl)ethyl)thio)-1,4-dihydroquinazoline dihydrochloride

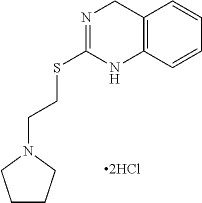 [SMILES: c1ccc2c(c1)CN=C(SCCN1CCCC1)N2 ; "·2HCl"]

Crude example 77 was obtained by centrifugation of the reaction mixture followed by trituration of the solid in MeCN (2×2 mL). The solid was then passed through an ISOLUTE® SCX-2 cartridge (DCM, then NH₃ 3 M in MeOH) and concentrated to dryness. To a solution of the resulting solid in DCM was added HCl in Et₂O (2.0 equiv) and, after evaporation to dryness, the residue was freeze-dried in water to afford a white solid (161 mg, 82%).

¹H-NMR (D₂O, 400 MHz) δ: 2.11-2.18 (m, 4H, 2 CH₂); 3.49 (bs, 4H, 2 N—CH₂); 3.65 (s, 4H, N—CH₂+S-CH₂); 4.83 (s, 2H, N—CH₂—Ar); 7.12 (dd, J7.8, 0.9 Hz, 1H, Ar); 7.25 (dd, J7.6, 0.9 Hz, 1H, Ar); 7.36 (dt, J7.6, 1.3 Hz, 1H, Ar); 7.42 (dt, J7.8, 1.3 Hz, 1H, Ar). M/Z (M+H)⁺: 262.1. Mp: 195-203° C.

### Example 78: 4,4-dimethyl-2-((2-(pyrrolidin-1-yl)ethyl)thio)-1,4-dihydroquinazoline dihydrochloride

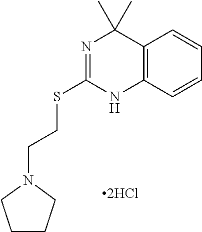 [SMILES: CC1(C)N=C(SCCN2CCCC2)Nc2ccccc21]

Crude example 78 was obtained by centrifugation of the reaction mixture. The supernatant was extracted with aqueous 1 N HCl and the resulting aqueous layer was freeze-dried. The residue was passed through an ISOLUTE® SCX-2 cartridge (MeOH, then NH₃ 3 M in MeOH) and concentrated to dryness. To a solution of the resulting solid in DCM was added HCl in Et₂O (2.0 equiv) and, after evaporation to dryness, the residue was freeze-dried in water.

Then further purification by preparative HPLC (column B, H₂O+0.1% HCOOH/MeCN+0.1% HCOOH 95:5 to 55:45) and freeze-drying with 1 N aqueous HCl (5 equiv) afforded an orange sticky solid (151 mg, 71%). M/Z (M+H)⁺: 290.2.

¹H-NMR (DMSO-d₆, 400 MHz) δ: 1.68 (s, 6H, 2 CH₃); 1.87-2.06 (m, 4H, 2 CH₂); 3.07 (bs, 2H, N—CH₂); 3.56 (bs, 4H, N—CH₂+S-CH₂); 3.83-3.93 (m, 2H, N—CH₂); 7.27-7.32 (m, 1H, Ar); 7.33-7.39 (m, 2H, 2 Ar); 7.43 (d, J7.8 Hz, 1H, Ar); 10.85 (bs, 1H, HCl salt); 11.09 (bs, 1H, HCl salt); 12.81 (bs, 1H, NH). M/Z (M+H)⁺: 290.2.

### Example 79: 2-bromo-7-chloro-3-(((1,4-dihydroquinazolin-2-yl)thio)methyl)-5H-thiazolo[2,3-b]quinazoline dihydrochloride

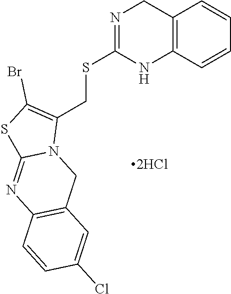 [SMILES: Clc1ccc2c(c1)CN1C(=N2)SC(Br)=C1CSC1=NCc2ccccc2N1]

Example 79 was isolated as a white solid (11 mg, 10% over 2 steps) by centrifugation of the reaction mixture followed by trituration of the solid in MeCN (2×2 mL) and in Et₂O (2×2 mL), followed by purification by preparative HPLC (column A, H₂O+0.1% HCOOH/MeCN+0.1% HCOOH 90:10 to 50:50) and freeze-drying with 1 N aqueous HCl (5 equiv).

¹H-NMR (DMSO-d₆, 400 MHz) δ: 4.76 (s, 4H, S—CH₂+N-CH₂); 5.54 (s, 2H, N—CH₂); 7.20-7.23 (m, 2H, 2 Ar); 7.27 (dt, J7.3, 1.0 Hz, 2H, 2 Ar); (d, J8.5 Hz, 1H, Ar); 7.22 (dt, J7.7, 1.3 Hz, 1H, Ar); 7.39 (dd, J8.5, 2.1 Hz, 1H, Ar); 11.29 (bs, 1H, HCl salt); 12.90 (bs, 1H, HCl salt); NH signal not observed. M/Z (M[³⁵Cl][⁷⁹Br]+H)⁺: 477.0. Mp>250° C.

### Example 80: 7-chloro-3-(((5-fluoro-1,4-dihydroquinazolin-2-yl)thio)methyl)-5H-thiazolo[2,3-b]quinazoline dihydrochloride

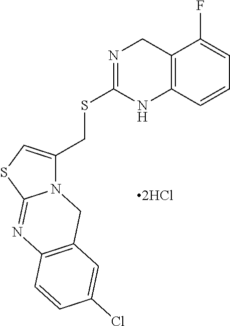 [SMILES: Fc1cccc2c1CN=C(SCC1=CSC3=Nc4ccc(Cl)cc4CN13)N2]

Example 80 was isolated as a white solid (38 mg, 56%) by centrifugation of the reaction mixture followed by trituration of the solid in EtOH (3×2 mL) and in Et₂O (2×2 mL).

¹H-NMR (DMSO-d₆+D₂O, 400 MHz) δ: 4.65 (s, 2H, S—CH₂); 4.70 (s, 2H, N—CH₂); 5.49 (s, 2H, N—CH₂); 6.89 (d, J8.0 Hz, 1H, Ar); 7.03-7.08 (m, 2H, 2 Ar); 7.28 (s, 1H, S—CH); 7.31-7.39 (m, 2H, 2 Ar); 7.42 (dd, J8.4, 2.4 Hz, 1H, Ar). M/Z (M[³⁵Cl]+H)⁺: 417.1. Mp>250° C.

### Example 81: 6-chloro-3-(((1,4-dihydroquinazolin-2-yl)thio)methyl)-5H-thiazolo[2,3-b]quinazoline dihydrochloride

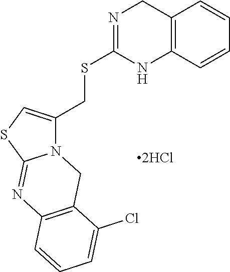 [SMILES: Clc1cccc2c1CN1C(CSC3=NCc4ccccc4N3)=CSC1=N2]

Example 81 was isolated as a white solid (90 mg, 83%) by centrifugation of the reaction mixture followed by trituration of the solid in EtOH (3×2 mL) and in Et₂O (2×2 mL).

¹H-NMR (DMSO-d₆, 400 MHz) δ: 4.68 (s, 2H, S—CH₂); 5.07 (s, 2H, N—CH₂); 5.53 (s, 2H, N—CH₂); 7.04 (d, J8.0 Hz, 1H, Ar); 7.24-7.40 (m, 6H, 6 Ar); 7.44 (s, 1H, S—CH); 11.20 (bs, 1H, HCl salt); 12.80 (bs, 1H, HCl salt); 13.77 (bs, 1H, NH). M/Z (M[³⁵Cl]+H)⁺: 399.1. Mp>250° C.

### Example 82: 3-(((1,4-dihydroquinazolin-2-yl)thio)methyl)-8-fluoro-5H-thiazolo[2,3-b]quinazoline dihydrochloride

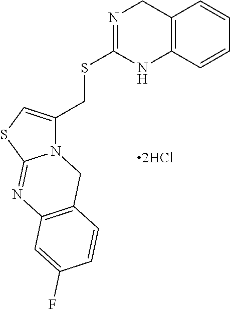 [SMILES: Fc1ccc2c(c1)N=C1SC=C(CSC3=NCc4ccccc4N3)N1C2]

Example 82 was isolated as a white solid (131 mg, 89%) by centrifugation of the reaction mixture followed by trituration of the solid in MeCN (2×2 mL) and in Et₂O (2×2 mL).

¹H-NMR (DMSO-d₆, 400 MHz) δ: 4.68 (s, 2H, S—CH₂); 4.97 (s, 2H, N—CH₂—Ar); 5.54 (s, 2H, N—CH₂—Ar); 6.92 (dd, J9.5, 2.5 Hz, 1H, Ar); 7.07 (td, J 8.6, 2.5 Hz, 1H, Ar); 7.21-7.35 (m, 5H, 5 Ar); 7.43 (s, 1H, S—CH); 11.18 (bs, 1H, HCl salt); 12.83 (bs, 1H, HCl salt); NH signal not observed. M/Z (M+H)⁺: 383.1. Mp>250° C.

### Example 83: 7-chloro-3-(((6-fluoro-1,4-dihydroquinazolin-2-yl)thio)methyl)-5H-thiazolo[2,3-b]quinazoline dihydrochloride

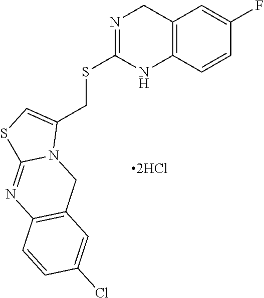 [SMILES: Fc1ccc2c(c1)CN=C(SCC1=CSC3=Nc4ccc(Cl)cc4CN13)N2]

Example 83 was isolated as a white solid (44 mg, 42%) by centrifugation of the reaction mixture followed by trituration of the solid in EtOH (3×2 mL) and in Et₂O (2×2 mL).

¹H-NMR (DMSO-d₆, 400 MHz) δ: 4.67 (s, 2H, S—CH₂); 4.95 (bs, 2H, N—CH₂); 5.56 (s, 2H, N—CH₂); 7.11 (d, J8.6 Hz, 1H, Ar); 7.14-7.21 (m, 2H, 2 Ar); 7.30-7.36 (m, 2H, S-CH+1 Ar); 7.43 (dd, J8.4, 2.4 Hz, 2H, 2 Ar); 11.18 (bs, 1H, HCl salt); 13.02 (bs, 1H, HCl salt); 13.64 (bs, 1H, NH). M/Z (M[³⁵Cl]+H)⁺: 417.1. Mp>250° C.

### Example 84: 3-(((1,4-dihydroquinazolin-2-yl)thio)methyl)-7-fluoro-5H-thiazolo[2,3-b]quinazoline dihydrochloride

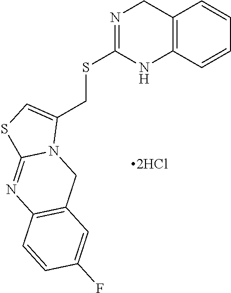 [SMILES: Fc1ccc2c(c1)CN1C(CSC3=NCc4ccccc4N3)=CSC1=N2]

Example 84 was isolated as a white solid (118 mg, 84%) by centrifugation of the reaction mixture followed by trituration of the solid in EtOH (3×2 mL) and in Et₂O (2×2 mL).

¹H-NMR (DMSO-d₆+D₂O, 400 MHz) δ: 4.65 (s, 2H, S—CH₂); 4.68 (s, 2H, N—CH₂); 5.48 (s, 2H, N—CH₂); 7.03-7.08 (m, 2H, 2 Ar); 7.17-7.27 (m, 5H, S-CH+4 Ar); 7.32 (t, J7.6 Hz, 1H, Ar). M/Z (M+H)⁺: 383.1. Mp>250° C.

### Example 85: 9-bromo-3-(((1,4-dihydroquinazolin-2-yl)thio)methyl)-5H-thiazolo[2,3-b]quinazoline dihydrochloride

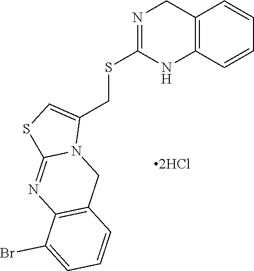 [SMILES: Brc1cccc2c1N=C1SC=C(CSC3=NCc4ccccc4N3)N1C2]

Example 85 was isolated as a white solid (72 mg, 86%) by centrifugation of the reaction mixture followed by trituration of the solid in EtOH (3×2 mL) and in Et₂O (2×2 mL).

¹H-NMR (DMSO-d₆+D₂O, 400 MHz) δ: 4.69 (s, 4H, S—CH₂+N-CH₂); 5.51 (s, 2H, N—CH₂); 7.09 (d, J8.0 Hz, 1H, Ar); 7.13 (t, J 8.0 Hz, 1H, Ar); 7.21 (d, J7.6 Hz, 1H, Ar); 7.24-7.28 (m, 3H, S-CH+2 Ar); 7.33 (t, J7.6 Hz, 1H, Ar); 7.61 (d, J 8.0 Hz, 1H, Ar). M/Z (M[⁷⁹Br]+H)⁺: 443.0. Mp>250° C.

### Example 86: 7-chloro-3-(((1,4-dihydroquinazolin-2-yl)thio)methyl)-9-fluoro-5H-thiazolo[2,3-b]quinazoline dihydrochloride

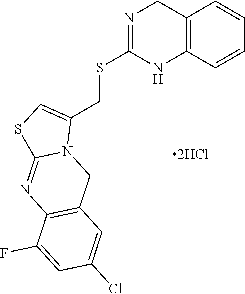 [SMILES: Fc1cc(Cl)cc2c1N=C1SC=C(CSC3=NCc4ccccc4N3)N1C2]

Example 86 was isolated as a white solid (96 mg, 90%) by centrifugation of the reaction mixture followed by trituration of the solid in EtOH (3×2 mL) and in Et₂O (2×2 mL).

¹H-NMR (DMSO-d₆+D₂O, 400 MHz) δ: 4.65 (s, 2H, S—CH₂); 4.69 (s, 2H, N—CH₂); 5.46 (s, 2H, N—CH₂); 7.09 (d, J8.0 Hz, 1H, Ar); 7.16 (s, 1H, S—CH); 7.18-7.22 (m, 2H, 2 Ar); 7.26 (td, J7.4, 1.2 Hz, 1H, Ar); 7.33 (td, J7.6, 1.4 Hz, 1H, Ar); 7.46 (dd, J10.2, 2.0 Hz, 1H, Ar). M/Z (M[³⁵Cl]+H)⁺: 417.0. Mp: 245-247° C.

### Example 87: 6-benzyl-3-(((4,4-dimethyl-1,4-dihydroquinazolin-2-yl)thio)methyl)-5,6-dihydroimidazo[2,1-b]thiazole dihydrochloride

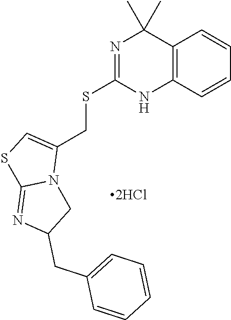 [SMILES: CC1(C)N=C(SCC2=CSC3=NC(Cc4ccccc4)CN23)Nc2ccccc21]

Crude example 87 was obtained by concentration of the reaction mixture. The residue was then dissolved in water (10 mL) and washed with EtOAc (2×10 mL). The resulting aqueous layer was freeze-dried to afford a white solid (122 mg, 75%)

¹H-NMR (DMSO-d₆, 400 MHz) δ: 1.61 (bs, 6H, 2 CH₃); 3.09-3.14 (m, 2H, CH₂-Ph); 4.32 (dd, J10.6, 7.0 Hz, 1H, N—CHₐH_{b}); 4.57 (t, J10.6 Hz, 1H, N—CHₐH_{b}); 4.77-5.00 (m, 2H, S—CH₂); 5.01-5.08 (m, 1H, N—CH); 6.94 (bs, 1H, S—CH); 7.22-7.42 (m, 9H, 9 Ar); 10.04 (bs, 1H, HCl salt); 11.17 (bs, 1H, HCl salt); 13.04 (bs, 1H, NH). M/Z (M+H)⁺: 421.3. Mp: 50-52° C.

### Example 88: 6-benzyl-3-(((4,5-dihydro-1H-benzo[d][1,3]diazepin-2-yl)thio)methyl)-5,6-dihydroimidazo[2,1-b]thiazole dihydrochloride

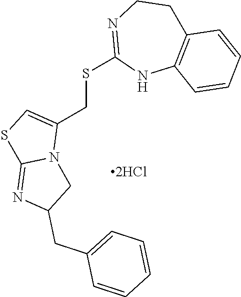 [SMILES: C1=C(CSC2=NCCc3ccccc3N2)N2CC(Cc3ccccc3)N=C2S1]

Crude example 88 was obtained by concentration of the reaction mixture. The residue was then dissolved in water (10 mL) and washed with DCM (2×10 mL). The resulting aqueous layer was freeze-dried to give a white solid (146 mg, 92%).

¹H-NMR (DMSO-d₆, 400 MHz) δ: 3.02-3.17 (m, 4H, CH₂-Ph+CH₂—Ar); 3.65 (bs, 2H, N—CH₂—CH₂—Ar); 4.31-4.35 (m, 1H, N—CHₐH_{b}); 4.58-4.79 (m, 3H, N—CHₐH_{b}+S-CH₂); 5.01-5.09 (m, 1H, N—CH); 6.86 (bs, 1H, S—CH); 7.17-7.39 (m, 8H, 8 Ar); 7.48-7.59 (m, 1H, Ar); 10.04 (bs, 1H, HCl salt); 11.33 (bs, 1H, HCl salt); 12.01 (bs, 1H, NH). M/Z (M+H)⁺: 407.2. Mp: 50-53° C.

### Example 89: 6-benzyl-3-(((7-fluoro-1,4-dihydroquinazolin-2-yl)thio)methyl)-5,6-dihydroimidazo[2,1-b]thiazole dihydrochloride

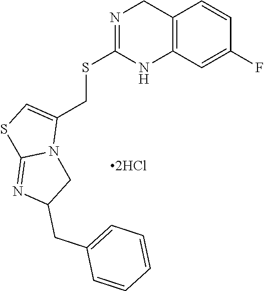 [SMILES: Fc1ccc2c(c1)NC(SCC1=CSC3=NC(Cc4ccccc4)CN13)=NC2]

Crude example 89 was obtained by precipitation of the reaction mixture with Et₂O (4 mL) followed by centrifugation. The solid was then triturated in Et₂O (2×2 mL), purified by preparative HPLC (column B, H₂O+0.1% HCOOH/MeCN+0.1% HCOOH 95:5 to 55:45) and freeze-dried with 1 N aqueous HCl (5 equiv) to afford a white solid (49 mg, 38%).

¹H-NMR (DMSO-d₆, 400 MHz) δ: 3.05-3.15 (m, 2H, CH₂-Ph); 4.25-4.30 (m, 1H, N—CHₐH_{b}); 4.51 (t, J10.6 Hz, 1H, N—CHₐH_{b}); 4.61 (bs, 2H, N—CH₂—Ar); 4.81 (bs, 2H, S—CH₂); 4.98-5.06 (m, 1H, N—CH); 6.97-7.37 (m, 9H, 8 Ar+S-CH); 10.01 (bs, 1H, HCl salt); 11.43 (bs, 1H, HCl salt); 13.17 (bs, 1H, NH signal). M/Z (M+H)⁺: 411.2. Mp: 125-130° C.

### Example 90: 2-((2-(azepan-1-yl)ethyl)thio)-1,4-dihydroquinazoline dihydrochloride

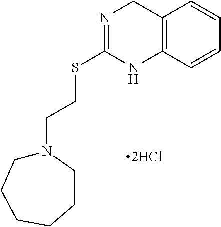 [SMILES: c1ccc2c(c1)CN=C(SCCN1CCCCCC1)N2]

Crude example 90 was obtained by concentration to dryness of the reaction mixture. The residue was then passed through an ISOLUTE® SCX-2 cartridge (DCM and MeOH, then NH₃ 3 M in MeOH) and concentrated to dryness. To a solution of the resulting solid in DCM was added HCl in Et₂O and, after evaporation to dryness, the residue was purified by flash chromatography (DCM 100% to DCM/MeOH 90:10, then DCM/[MeOH+1% NH₄OH 28% aq.] 80:20)), then freeze-dried in a mixture of water and 1 N aqueous HCl (5 equiv) to afford an orange sticky solid (109 mg, 82%). ¹H-NMR (DMSO-d₆, 400 MHz) δ: 1.55-1.70 (m, 4H, 2 CH₂); 1.81-1.89 (m, 4H, 2 CH₂); 3.12-3.25 (m, 2H, N—CH₂); 3.33- 3.45 (m, 4H, 2 N—CH₂); 3.77-3.85 (m, 2H, S—CH₂); 4.74 (s, 2H, N—CH₂—Ar); 7.19-7.21 (m, 1H, Ar); 7.24-7.25 (m, 2H, 2 Ar); 7.31-7.37 (m, 1H, Ar); 10.60 (bs, 1H, HCl salt); 10.83 (bs, 1H, HCl salt); 10.51 (bs, 1H, NH). M/Z (M+H)⁺: 290.1.

### Example 91: 2-((2-(piperidin-1-yl)ethyl)thio)-1,4-dihydroquinazoline dihydrochloride

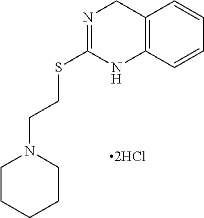 [SMILES: c1ccc2c(c1)CN=C(SCCN1CCCCC1)N2]

Crude example 91 was obtained by concentration to dryness of the reaction mixture. The residue was then passed through an ISOLUTE® SCX-2 cartridge (DCM and MeOH, then NH₃ 3 M in MeOH) and concentrated to dryness. To a solution of the resulting yellow oil in DCM was added HCl in Et₂O and, after evaporation to dryness, the residue was purified by flash chromatography (DCM 100% to DCM/MeOH 90:10, then DCM/[MeOH+1% NH₄OH 28% aq.]80:20), then freeze-dried in a mixture of water and 1 N aqueous HCl (5 equiv). The resulting solid was suspended in an aqueous saturated NaHCO₃ solution (10 mL), extracted with EtOAc (3×10 mL), dried over magnesium sulfate, concentrated to dryness and freeze-dried in a mixture of water and 1 N aqueous HCl (5 equiv) to obtain a colorless solid (108 mg, 73%).

¹H-NMR (DMSO-d₆, 400 MHz) δ: 1.31-1.48 (m, 1H, CHₐH_{b}); 1.66-1.83 (m, 5H, 2 CH₂+CHₐH_{b}); 2.85-3.04 (m, 2H, N—CH₂); 3.45-3.56 (m, 4H, 2 N—CH₂); 3.78-3.87 (m, 2H, S—CH₂); 4.73 (s, 2H, CH₂—Ar); 7.20-7.27 (m; 3H, 3 Ar); 7.30-7.36 (m, 1H, Ar); 10.63 (bs, 1H, HCl salt); 10.91 (bs, 1H, HCl salt); 10.61 (bs, 1H, NH). M/Z (M+H)⁺: 276.1.

### Example 92: 3-(((8-bromo-1,4-dihydroquinazolin-2-yl)thio)methyl)-7-chloro-5H-thiazolo[2,3-b]quinazoline dihydrochloride

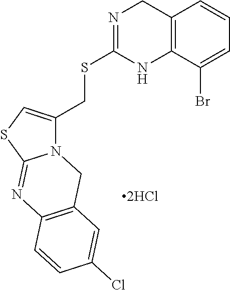 [SMILES: Clc1ccc2c(c1)CN1C(CSC3=NCc4cccc(Br)c4N3)=CSC1=N2]

Example 92 was isolated as a white solid (25 mg, 20%) by centrifugation of the reaction mixture followed by trituration of the solid in EtOH (3×2 mL), in MeOH (2×2 mL) and in Et₂O (2×2 mL).

¹H-NMR (DMSO-d₆+D₂O, 400 MHz) δ: 4.43 (s, 2H, S—CH₂); 4.54 (s, 2H, N—CH₂); 5.49 (s, 2H, N—CH₂); 6.91 (t, J 7.8 Hz, 1H, Ar); 6.97-7.01 (m, 2H, 2 Ar); 7.22 (s, 1H, S—CH); 7.28 (d, J 2.2 Hz, 1H, Ar); 7.38-7.44 (m, 2H, 2 Ar). M/Z (M[³⁵Cl][⁷⁹Br]+H)⁺: 477.0. Mp>250° C.

### Example 93: 6-benzyl-3-(((3-butyl-3,4-dihydroquinazolin-2-yl)thio)methyl)-5,6-dihydroimidazo[2,1-b]thiazole dihydrochloride

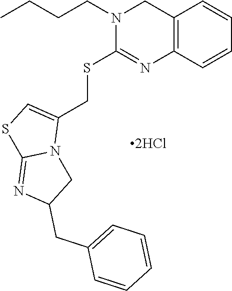 [SMILES: CCCCN1Cc2ccccc2N=C1SCC1=CSC2=NC(Cc3ccccc3)CN12]

Crude example 93 was obtained by concentration of the reaction mixture. The residue was then dissolved in water (15 mL) and washed with EtOAc (2×10 mL). The resulting aqueous layer was freeze-dried and purified by preparative HPLC (column A, H₂O+0.1% HCOOH/MeCN+0.1% HCOOH 90:10 to 50:50) and freeze-dried with 1 N aqueous HCl (3 mL) to give a white solid (41 mg, 47%).

¹H-NMR (DMSO-d₆, 400 MHz) δ: 0.92 (t, J7.4 Hz, 3H, CH₃); 1.25-1.35 (m, 2H, CH₃—CH₂); 1.59-1.67 (m, 2H, CH₃—CH₂—CH₂); 3.04-3.113 (m, 2H, CH₂-Ph); 3.72 (bs, 2H, N—CH₂—CH₂); 4.24 (dd, J10.6, 7.0 Hz, 1H, N—CHₐH_{b}); 4.50 (t, J 10.6 Hz, 1H, N—CHₐH_{b}); 4.75-4.86. (m, 2H, S—CH₂); 4.97-5.24 (m, 3H, N-CH+N-CH₂—Ar); 6.98 (bs, 1H, S—CH); 7.12-7.39 (m, 8H, 8 Ar); 7.60 (bs, 1H, Ar); 10.07 (s, 1H, HCl salt); 13.10 (bs, 1H, HCl salt). M/Z (M+H)⁺: 449.2. Mp: 40-44° C.

### Example 94: 6-(4-chlorobenzyl)-3-(((1,4-dihydroquinazolin-2-yl)thio)methyl)-5,6-dihydroimidazo[2,1-b]thiazole dihydrochloride

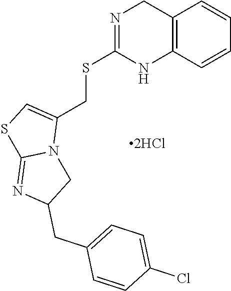 [SMILES: Clc1ccc(CC2CN3C(CSC4=NCc5ccccc5N4)=CSC3=N2)cc1]

Crude example 94 was obtained by precipitation of the reaction mixture with Et₂O (2 mL) followed by centrifugation. The solid was then triturated in Et₂O (2×2 mL), purified by preparative HPLC (column B, H₂O+0.1% HCOOH/MeCN+0.1% HCOOH 95:5 to 55:45) and freeze-dried with 1 N aqueous HCl (5 equiv) to obtain a yellow solid (12 mg, 21%). ¹H-NMR (DMSO-d₆, 400 MHz) δ: 3.10 (d, J 6.6 Hz, 2H, CH₂—Ar); 4.29 (dd, J10.6, 6.9 Hz, 1H, N—CHₐH_{b}); 4.52 (t, J 10.6 Hz, 1H, N—CHₐH_{b}); 4.66 (s, 2H, N—CH₂); 4.82-4.92 (m, 2H, S—CH₂); 4.98-5.06 (m, 1H, N—CH); 7.01 (s, 1H, S—CH); 7.21-7.28 (m, 2H, 2 Ar); 7.30-7.35 (m, 2H, 2 Ar); 7.36-7.43 (m, 4H, 4 Ar); 10.11 (s, 1H, HCl salt); 11.33 (bs, 1H, HCl salt); 12.99 (bs, 1H, NH). M/Z (M[³⁵Cl]+H)⁺: 427.2. Mp: 190-200° C.

### Example 95: 3-(((1,4-dihydroquinazolin-2-yl)thio)methyl)-5,5-dimethyl-5H-thiazolo[2,3-b]quinazoline dihydrochloride

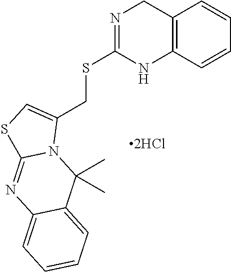 [SMILES: CC1(C)c2ccccc2N=C2SC=C(CSC3=NCc4ccccc4N3)N21]

Crude example 95 was obtained by centrifugation of the reaction mixture followed by trituration of the solid in MeCN (2×2 mL) and in Et₂O (2×2 mL). It was then purified by preparative HPLC (column A, H₂O+0.1% HCOOH/MeCN+0.1% HCOOH 95:5 to 55:45) and freeze-dried with 1 N aqueous HCl to obtain a white solid (41 mg, 26%).

¹H-NMR (DMSO-d₆, 400 MHz) δ: 1.65 (s, 6H, 2 CH₃); 5.06 (s, 2H, S—CH₂); 5.61 (s, 2H, N—CH₂—Ar); 7.09 (d, J7.8 Hz, 1H, Ar); 7.22-7.30 (m, 4H, 3 Ar+S-CH); 7.32-7.42 (m, 4H, 4 Ar); 11.14 (bs, 1H, HCl salt); 12.93 (bs, 1H, HCl salt); 13.41 (bs, 1H, NH). M/Z (M+H)⁺: 393.1. Mp: 180-185° C.

### Example 96: 3-(((3,4-dihydroquinazolin-2-ylthio)methyl)benzo[4,5]imidazo[2,1-b]thiazole hydrochloride

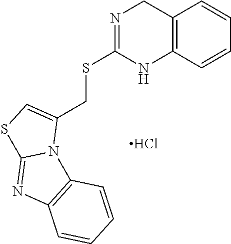 [SMILES: c1ccc2c(c1)CN=C(SCc1csc3nc4ccccc4n13)N2 ; Cl]

Example 96 was isolated as a beige solid (172 mg, 89%) by centrifugation of the reaction mixture followed by trituration of the solid in MeCN (3×2 mL) and in Et₂O (2×3 mL).

¹H-NMR (DMSO-d₆, 400 MHz) δ: 4.80 (s, 2H, N—CH₂); 5.63 (s, 2H, S—CH₂); 7.08 (d, J8.0 Hz, 1H, Ar); 7.18 (s, 1H, S—CH); 7.23 (t, J8.0 Hz, 1H, Ar); 7.27-7.31 (m, 3H, 3 Ar); 7.37 (t, J 8.0 Hz, 1H, Ar); 7.55-7.60 (m, 2H, 2 Ar); 13.33 (bs, 1H, NH); HCl salt signal not observed. M/Z (M+H)⁺: 351.1. Mp>250° C.

### Example 97: 3-(((3,4-dihydroquinazolin-2-yl)thio)methyl)-6,7-dimethoxybenzo[4,5]imidazo[2,1-b]thiazole hydrochloride

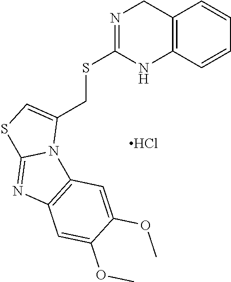 [SMILES: COc1cc2nc3scc(CSC4=NCc5ccccc5N4)n3c2cc1OC ; Cl]

Example 97 was isolated as a white solid (65 mg, 51%) by filtration of the reaction mixture and washing of the solid with MeCN, followed by purification by preparative HPLC (column A, H₂O+0.1% HCOOH/MeCN+0.1% HCOOH 95:5 to 55:45) and freeze-drying with 1 N aqueous HCl (5 equiv).

¹H-NMR (DMSO-d₆, 400 MHz) δ: 3.82 (s, 6H, 2 CH₃); 4.70 (s, 2H, S—CH₂); 5.63 (s, 2H, N—CH₂—Ar); 7.03 (s, 1H, S—CH); 7.09 (s, 1H, Ar); 7.10 (s, 2H, 2 Ar); 7.24 (dt, J 7.7, 1.1 Hz, 1H, Ar); 7.30 (dd, J7.7, 1.1 Hz, 1H, Ar); 7.38 (dt, J7.7, 1.1 Hz, 1H, Ar); 13.35 (bs, 1H, NH); HCl salt not observed. M/Z (M+H)⁺: 411.1. Mp: 238-240° C.

### Example 98: 4,4-dimethyl-2-((1-methylpyrrolidin-3-yl)thio)-1,4-dihydroquinazoline dihydrochloride

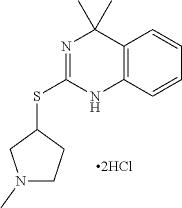 [SMILES: CN1CCC(SC2=NC(C)(C)c3ccccc3N2)C1]

Crude example 98 was obtained by hydrolysis of the reaction mixture with water (20 mL) followed by extraction with DCM (2×15 mL). The combined organic layers were extracted with aqueous 1 N HCl (10 mL), and the resulting aqueous layer was freeze-dried. The resulting light-orange oil was passed through an ISOLUTE® SCX-2 cartridge (MeOH, then NH₃ 3 M in MeOH) and concentrated to dryness. To a solution of the resulting solid in DCM (2 mL) was added HCl in Et₂O (2.0 equiv) and, after evaporation to dryness, the residue was purified by preparative HPLC (column B, H₂O+0.1% HCOOH/MeCN+0.1% HCOOH 95:5 to 55:45) and freeze-dried with 1 N aqueous HCl (5 equiv) to obtain a colorless sticky solid (196 mg, 64%).

¹H-NMR (DMSO-d₆, 400 MHz) δ: 1.67 (s, 3H, CH₃); 1.68 (s, 3H, CH₃); 2.01-2.23 (m, 1H, CH—CHₐH_{b}); 2.70-2.94 (m, 4H, N—CH₃+CH—CHₐH_{b}); 3.07-3.25 (m, 1.5H, one rotamer of N—CH₂); 3.63-3.70 (m, 2H, N—CH₂); 4.03-4.14 (m, 0.5H, other rotamer of N—CH₂); 4.75-4.86 (m, 0.5H, one rotamer of S—CH); 5.06-5.15 (m, 0.5H, other rotamer of S—CH); 7.28-7.46 (m, 4H, 4 Ar); 11.00 (bs, 1H, HCl salt); 11.36-11.54 (m, 1H, HCl salt); 12.99 (bs, 1H, NH). M/Z (M+H)⁺: 276.1.

### Example 99: 6-benzyl-3-(((1-butyl-1,4-dihydroquinazolin-2-yl)thio)methyl)-5,6-dihydroimidazo[2,1-b]thiazole dihydrochloride

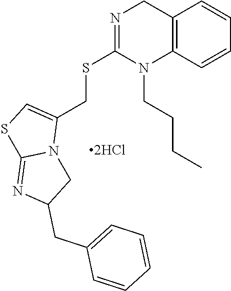 [SMILES: CCCCN1C(SCC2=CSC3=NC(Cc4ccccc4)CN23)=NCc2ccccc21]

Crude example 99 was obtained by hydrolysis of the reaction mixture with water (2 mL) followed by extraction with EtOAc (2×10 mL). The combined organic layers were washed with brine (10 mL), dried over magnesium sulfate and concentrated to dryness. The resulting orange oil was purified twice by preparative HPLC (column A, H₂O+0.1% HCOOH/MeCN+0.1% HCOOH 80:20 to 60:40 then column B, H₂O+0.1% HCOOH/MeCN+0.1% HCOOH 95:5 to 55:45) and freeze-dried with 1 N aqueous HCl (5 equiv) to obtain a white solid (10 mg, 7%).

¹H-NMR (DMSO-d₆, 400 MHz) δ: 0.90 (t, J7.3 Hz, 3H, CH₃); 1.27-1.39 (m, 2H, CH₂); 1.58-1.68 (m, 2H, CH₂); 3.06-3.16 (m, 2H, CH₂-Ph); 4.14 (bs, 2H, CH₂—Ar); 4.24-4.28 (m, 1H, N—CHₐH_{b}); 4.49-4.61 (m, 3H, N—CHₐH_{b}+N-CH₂); 4.79-5.07 (m, 3H, N-CH+S-CH₂); 7.03 (bs, 1H, S—CH); 7.24-7.42 (m, 9H, 9 Ar); 10.09 (bs, 1H, HCl salt); 11.71 (bs, 1H, HCl salt). M/Z (M+H)⁺: 449.4. Mp: 50-58° C.

### Example 100: 2-((1-methylpyrrolidin-3-yl)thio)-1,4-dihydroquinazoline dihydrochloride

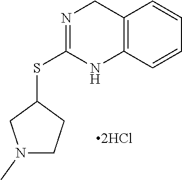 [SMILES: CN1CCC(SC2=NCc3ccccc3N2)C1]

Crude example 100 was obtained by hydrolysis of the reaction mixture with water (10 mL) followed by extraction with EtOAc (2×10 mL). The combined organic layers were extracted with 1 N aqueous HCl (10 mL), and the resulting aqueous layer was washed with EtOAc (2×10 mL) and freeze-dried. The resulting yellow solid was purified by preparative HPLC (column B, H₂O+0.1% HCOOH/MeCN+0.1% HCOOH 95:5 to 55:45) and freeze-dried with 1 N aqueous HCl (5.0 equiv) to obtain a yellow hygroscopic solid (118 mg, 37%).

¹H-NMR (DMSO-d₆, 400 MHz) δ: 2.01-2.12 (m, 0.5H, one rotamer of CHₐH_{b}); 2.16-2.27 (m, 0.5H, other rotamer of CHₐH_{b}); 2.72-2.90 (m, 4H, N—CH₃+CHₐH_{b}); 3.07-3.28 (1.5H, N—CHₐH_{b}+one rotamer of N—CHₐH_{b}); 3.63-6.73 (m, 2H, N—CH₂); 4.08-4.18 (m, 0.5H, other rotamer of N—CHₐH_{b}); 4.61-4.70 (m, 0.5H, one rotamer of S—CH); 4.72 (s, 2H, N—CH₂); 4.88-4.97 (m, 0.5H, other rotamer of S—CH); 7.23-7.28 (m, 3H, 3 Ar); 7.31-7.37 (m, 1H, Ar); 10.89-11.19 (m, 1H, HCl salt); 11.31-11.56 (m, 1H, HCl salt); 12.77 (bs, 1H, NH). M/Z (M+H)⁺: 248.1.

### Example 101: 2-((1-phenylpyrrolidin-3-yl)thio)-1,4-dihydroquinazoline hydrochloride

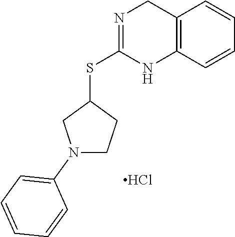 [SMILES: c1ccc(N2CCC(SC3=NCc4ccccc4N3)C2)cc1 ; Cl]

Crude example 101 was obtained by hydrolysis of the reaction mixture with water (5 mL) at 0° C. followed by extraction with DCM (2×5 mL). The combined organic layers were extracted with 1 N aqueous HCl (2×10 mL), and the resulting aqueous layer was freeze-dried, purified by preparative HPLC (column C, H₂O+0.1% HCOOH/MeCN+0.1% HCOOH 95:5 to 0:100) and freeze-dried with 1 N aqueous HCl (2.0 equiv) to obtain a beige solid (21 mg, 10% over 2 steps).

¹H-NMR (DMSO-d₆, 300 MHz) δ: 2.12-2.22 (m, 1H, CHₐH_{b}); 2.55-2.62 (m, 1H, CHₐH_{b}); 3.30-3.38 (m, 1H, N—CHₐH_{b}); 3.40-3.50 (m, 2H, N—CH₂); 3.73-3.79 (m, 1H, N—CHₐH_{b}); 4.72-4.80 (m, 3H, N—CH₂—Ar+S-CH); 6.57-6.69 (m, 3H, 3 Ar); 7.14-7.21 (m, 3H, 3 Ar); 7.23-7.28 (m, 2H, 2 Ar); 7.30-7.38 (m, 1H, Ar); 10.75 (bs, 1H, HCl salt); 12.47 (bs, 1H, NH). M/Z (M+H)⁺: 310.1.

### Example 102: 2-((1-(2,2-difluoroethyl)pyrrolidin-3-yl)thio)-1,4-dihydroquinazoline hydrochloride

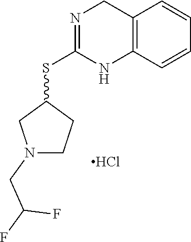 [SMILES: FC(F)CN1CCC(SC2=NCc3ccccc3N2)C1 ; Cl]

Crude example 102 was obtained by hydrolysis of the reaction mixture with water (2 mL) followed by extraction with DCM (2×10 mL). The combined organic layers were extracted with 1 N aqueous HCl (2×10 mL), and the resulting aqueous layer was freeze-dried, purified by preparative HPLC (column B, H₂O+0.1% HCOOH/MeCN+0.1% HCOOH 95:5 to 55:45) and freeze-dried with 1 N aqueous HCl (2.0 equiv). The resulting residue was dissolved in water (15 mL) and washed with DCM (2×10 mL), then the aqueous layer was freeze-dried and purified by Sephadex LH20 (MeOH 100%). Then HCl in Et₂O (2 equiv) was added to the residue which was concentrated to dryness and freeze-dried in water to obtain a yellow hygroscopic solid (30 mg, 14% over 2 steps).

¹H-NMR (DMSO-d₆, 300 MHz) δ: 2.00-2.16 (m, 1H, CHₐH_{b}); 2.59-2.70 (m, 1H, CHₐH_{b}); 2.99-3.63 (m, 6H, 3 N—CH₂); 4.57-4.75 (m, 3H, N—CH₂—Ar+S-CH); 6.44 (t, J 55.9 Hz, 1H, F—CH); 7.18-7.29 (m, 3H, 3 Ar); 7.29-7.38 (m, 1H, Ar); 11.05 (bs, 1H, HCl salt); 12.68 (b, 1H, NH). M/Z (M+H)⁺: 298.1.

### Example 103: 6-chloro-2-((1-methylpyrrolidin-3-yl)thio)-1,4-dihydroquinazoline dihydrochloride

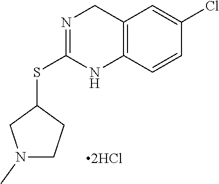 [SMILES: CN1CCC(SC2=NCc3cc(Cl)ccc3N2)C1]

Crude example 103 was obtained by hydrolysis of the reaction mixture at 0° C. with water (25 mL), followed by extraction with DCM (2×5 mL). The combined organic layers were extracted with 1 N aqueous HCl (2×10 mL), and the resulting combined aqueous layers were freeze-dried, purified by preparative HPLC (column B, H₂O+0.1% HCOOH/MeCN+0.1% HCOOH 95:5 to 55:45) and freeze-dried with 1 N aqueous HCl (2.0 equiv). The residue was dissolved in water (5 mL) and an aqueous saturated solution of NaHCO₃ (5 mL), then extracted with DCM (2×30 mL) and dried over magnesium sulfate. HCl in Et₂O (2.0 equiv) was added to the combined organic extracts that were concentrated to dryness. The residue was freeze-dried in water (10 mL) to obtain a white hygroscopic solid (30 mg, 17% over 2 steps).

¹H-NMR (DMSO-d₆, 300 MHz) δ: 1.99-2.23 (m, 1H, CHₐH_{b}); 2.57-2.65 (m, 1H, CHₐH_{b}); 2.84 (s, 3H, N—CH₃); 3.02-3.28 (m, 1.5H, N—CHₐH_{b}+one rotamer of N—CHₐH_{b}); 3.57-3.72 (m, 2H, N—CH₂); 4.06-4.19 (m, 0.5H, other rotamer of N—CHₐH_{b}); 4.57-4.68 (m, 0.5H, one rotamer of S—CH); 4.71 (s, 2H, N—CH₂—Ar); 4.87-4.97 (m, 0.5H, other rotamer of S—CH); 7.24-7.31 (m, 1H, Ar); 7.36-7.43 (m, 2H, 2 Ar); 10.98-11.56 (m, 2H, 2 HCl salts); 12.94 (bs, 1H, NH). M/Z (M[³⁵Cl]+H)⁺: 282.0.

### Example 104: 2-((1-ethylpyrrolidin-3-yl)thio)-1,4-dihydroquinazoline dihydrochloride

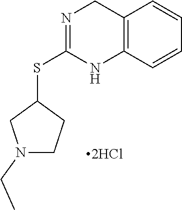 [SMILES: CCN1CCC(SC2=NCc3ccccc3N2)C1]

Crude example 104 was obtained by hydrolysis of the reaction mixture with water (5 mL) followed by extraction with DCM (2×10 mL). The combined organic layers were extracted with 1 N aqueous HCl (2×10 mL), and the resulting combined aqueous layers were freeze-dried, purified by preparative HPLC (column B, H₂O+0.1% HCOOH/MeCN+0.1% HCOOH 95:5 to 55:45) and freeze-dried with 1 N aqueous HCl (2.0 equiv). The residue was dissolved in water (10 mL) and washed with DCM (2×10 mL), then the aqueous layer was freeze-dried. The residue was dissolved in water (5 mL) and an aqueous saturated solution of NaHCO₃ (5 mL), then extracted with DCM (2×30 mL) and dried over magnesium sulfate. HCl in Et₂O (2.0 equiv) was added to the combined organic extracts that were concentrated to dryness. The residue was freeze-dried in water (10 mL) to obtain a white hygroscopic solid (8 mg, 4% over 2 steps). ¹H-NMR (D₂O, 300 MHz) δ: 1.22 (t, J7.3 Hz, 3H, CH₂—CH₃); 1.95-2.18 (m, 1H, CHₐH_{b}); 2.57-2.65 (m, 1H, CHₐH_{b}); 3.11-3.32 (m, 3H, N—CH₂—CH₃+N-CHₐH_{b}); 3.56-3.64 (m, 2H, N—CH₂); 3.94-4.29 (m, 1H, N—CHₐH_{b}); 4.34-4.56 (m, 1H, S—CH); 4.66 (s, 2H, N—CH₂—Ar); 7.00-7.09 (m, 1H, Ar); 7.14-7.23 (m, 2H, 2 Ar); 7.25-7.33 (m, 1H, Ar). M/Z (M+H)⁺: 262.2.

### Example 105: 2-((1-methylpyrrolidin-3-yl)thio)-4,5-dihydro-1H-benzo[d][1,3]diazepine dihydrochloride

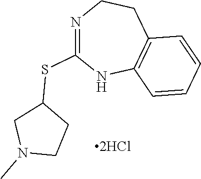 [SMILES: CN1CCC(SC2=NCCc3ccccc3N2)C1 ; "·2HCl"]

Crude example 105 was obtained by hydrolysis of the reaction mixture with water (20 mL) and extraction with EtOAc (2×30 mL). The combined organic layers were dried over magnesium sulfate and concentrated to dryness. The crude was purified by flash chromatography (DCM 100% to DCM/MeOH 80:20), then dissolved in water and 1 N aqueous HCl. The resulting aqueous solution was washed with DCM (20 mL) and EtOAc (20 mL) and freeze-dried. The residue was then further purified by preparative HPLC (column B, H₂O+0.1% HCOOH/MeCN+0.1% HCOOH 95:5) and freeze-dried with 1 N aqueous HCl (2.0 equiv) to obtain a white solid (77 mg, 27%).

¹H-NMR (D₂O, 300 MHz) δ: 2.15-2.27 (m, 0.6H, one rotamer of CHₐH_{b}); 2.32-2.43 (m, 0.4H, other rotamer of CHₐH_{b}); 2.64-2.78 (m, 0.4H, one rotamer of CHₐH_{b}); 2.83-2.95 (m, 0.6H, other rotamer of CHₐH_{b}); 3.02 (s, 1.8H, one rotamer of N—CH₃); 3.06 (s, 1.2H, other rotamer of N—CH₃); 3.23-3.26 (m, 2H, CH₂); 3.29-3.36 (m, 1H, N—CHₐH_{b}); 3.40-3.50 (m, 0.4H, one rotamer of N—CHₐH_{b}); 3.69-3.76 (m, 0.6H, other rotamer of N—CHₐH_{b}); 3.81-3.98 (m, 3.6H, N—CH₂+N-CHₐH_{b}+one rotamer of N—CHₐH_{b}); 4.24-4.30 (m, 0.4H, other rotamer of N—CHₐH_{b}); 4.39-4.48 (m, 0.4H, one isomer of S—CH); 4.58-4.67 (m, 0.6H, other isomer of S—CH); 7.27-7.42 (m, 4H, 4 Ar). M/Z (M+H)⁺: 262.2.

### Example 106: 3-((1-phenylpyrrolidin-3-yl)thio)-2,5-dihydro-1H-benzo[e][1,3]diazepine hydrochloride

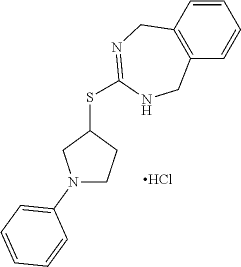 [SMILES: c1ccc(N2CCC(SC3=NCc4ccccc4CN3)C2)cc1 ; Cl]

Crude example 106 was obtained by addition of MeOH to the reaction mixture and elution of the resulting solution through an ISOLUTE® SCX-2 cartridge (MeOH, then NH₃3.5 N in MeOH), followed by purification of the resulting crude by flash chromatography (KPNH, CyHex 100% to CyHex/EtOAc 60:40). To a solution of the resulting residue in DCM was added HCl in Et₂O (5.0 equiv) and, after evaporation to dryness, the resulting residue was further purified by preparative HPLC (column C, H₂O+0.1% HCOOH/MeCN+0.1% HCOOH 70:30) and freeze-dried with 1 N aqueous HCl (2.0 equiv) to obtain a green oil (35 mg, 21% over 2 steps).

¹H-NMR (DMSO-d₆, 300 MHz) δ: 1.93-2.03 (m, 1H, CHₐH_{b}); 2.40-2.47 (m, 1H, CHₐH_{b}); 3.20-3.42 (m, 3H, N—CHₐH_{b}+N-CH₂); 3.66 (dd, J10.9, 6.1 Hz, 1H, N—CHₐH_{b}); 4.56-4.63 (m, 1H, S—CH); 4.79 (s, 2H, 2 N—CHₐH_{b}—Ar); 4.80 (s, 2H, 2 N—CHₐH_{b}—Ar); 6.50-6.55 (m, 2H, 2 Ar); 6.61-6.66 (m, 1H, Ar); 7.13-7.20 (m, 2H, 2 Ar); 7.38-7.44 (m, 4H, 4 Ar); 10.38-10.41 (m, 2H, NH+HCl salt). M/Z (M+H)⁺: 324.3.

### Example 107: 2-((1-phenylpyrrolidin-3-yl)thio)-4,5-dihydro-1H-benzo[d][1,3]diazepine

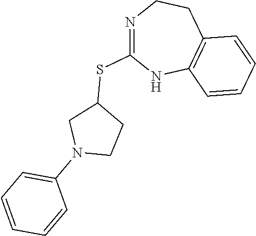 [SMILES: c1ccc(N2CCC(SC3=NCCc4ccccc4N3)C2)cc1]

Crude example 107 was obtained by hydrolysis of the reaction mixture with water (30 mL) and extraction with EtOAc (2×30 mL). The combined organic layers were dried over magnesium sulfate and concentrated to dryness. The crude was purified thrice by flash chromatography (CyHex 100% to CyHex/EtOAc 75:25, then 20 μm, CyHex 100% to CyHex/EtOAc 75:25, then KPNH, CyHex 100% to CyHex/EtOAc 0:100) and freeze-dried in MeCN and water to obtain a white solid (45 mg, 32% over 2 steps).

¹H-NMR (DMSO-d₆, 300 MHz) δ: 1.98-2.09 (m, 1H, CHₐH_{b}); 2.40-2.47 (m, 1H, CHₐH_{b}); 2.91-2.94 (m, 2H, Ar—CH₂); 3.23-3.28 (m, 2H, N—CH₂); 3.35-3.45 (m, 3H, N—CHₐH_{b}+N-CH₂); 3.79 (dd, J 10.1, 6.8 Hz, 1H, N—CHₐH_{b}); 4.22-4.31 (m, 1H, S—CH); 6.52-6.55 (m, 2H, 2 Ar); 6.60 (t, J7.3 Hz, 1H, Ar); 6.84 (td, J7.3, 1.4 Hz, 1H, Ar); 6.98-7.02 (m, 2H, 2 Ar); 7.07-7.12 (m, 1H, Ar); 7.13-7.19 (m, 2H, 2 Ar); 7.57 (t, J 4.0 Hz, 1H, NH). M/Z (M+H)⁺: 324.2.

### Example 108: 2-(((1-methylpyrrolidin-2-yl)methyl)thio)-1,4-dihydroquinazoline dihydrochloride

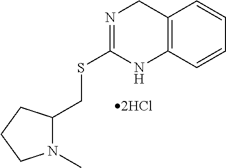 [SMILES: CN1CCCC1CSC1=NCc2ccccc2N1]

Crude example 108 was obtained by centrifugation of the reaction mixture. The solid was then passed through an ISOLUTE® SCX-2 cartridge (DCM and MeOH, then NH₃ 7 M in MeOH) and concentrated to dryness. To a solution of the resulting yellow oil in DCM was added HCl in Et₂O and, after evaporation to dryness, the residue was purified by preparative HPLC (column B, H₂O+0.1% HCOOH/MeCN+0.1% HCOOH 95:5 to 55:45) and freeze-dried with 1 N aqueous HCl (5 equiv) to obtain a yellow hygroscopic solid (16 mg, 11%).

¹H-NMR (DMSO-d₆, 400 MHz) δ: 1.79-1.88 (m, 1H, CH—CHₐH_{b}); 1.90-2.04 (m, 2H, CH₂); 2.69-2.32 (m, 0.5H, one rotamer of CH—CHₐH_{b}); 2.74-2.86 (m, 0.5H, other rotamer of CH—HₐH_{b}); 2.92 (s, 3H, N—CH₃); 3.01-3.11 (m, 1H, N—CH); 3.54-3.61 (m, 1H, one rotamer of S—CH₂); 3.66-3.73 (m, 2H, N—CH₂); 4.13-4.29 (m, 1H, other rotamer of S—CH₂); 4.72 (s, 2H, N—CH₂—Ar); 7.21-7.28 (m, 3H, 3 Ar); 7.31-7.36 (m, 1H, Ar); 11.13 (bs, 1H, HCl salt); 12.75 (bs, 1H, HCl salt); NH signal not observed. M/Z (M+H)⁺: 262.0.

### Example 109: (S)-6-((1H-indol-3-yl)methyl)-3-(((1,4-dihydroquinazolin-2-yl)thio)methyl)-5,6-dihydroimidazo[2,1-b]thiazole dihydrochloride

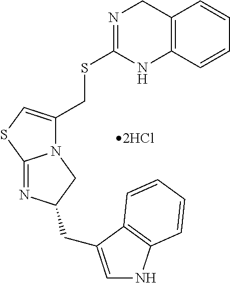 [SMILES: C1=C(CSC2=NCc3ccccc3N2)N2C[C@H](Cc3c[nH]c4ccccc34)N=C2S1]

Crude example 109 was obtained by precipitation of the reaction mixture with Et₂O (2 mL) followed by centrifugation and trituration of the solid in Et₂O (2×2 mL). The crude was then purified by preparative HPLC (column B, H₂O+0.1% HCOOH/MeCN+0.1% HCOOH 95:5 to 55:45) and freeze-dried with 1 N aqueous HCl (5.0 equiv) to afford a white solid (7 mg, 8% over 3 steps).

¹H-NMR (DMSO-d₆+D₂O, 400 MHz) δ: 3.11-3.22 (m, 2H, CH₂—Ar); 4.18-4.54 (m, 1H, N—CHₐH_{b}); 4.34-4.50 (m, 3H, N—CHₐH_{b}+S-CH₂); 4.61 (s, 2H, N—CH₂); 5.01-5.09 (m, 1H, N—CH); 6.76 (s, 1H, S—CH); 6.99-7.03 (m, 2H, 2 Ar); 7.09 (t, J15.0, 8.0 Hz, 1H, Ar); 7.15 (d, J8.0 Hz, 1H, Ar); 7.19-7.24 (m, 2H, 2 Ar); 7.29 (t, J15.0, 8.0 Hz, 1H, Ar); 7.36 (d, J 8.0 Hz, 1H, Ar). M/Z (M+H)⁺: 432.2. Mp: 180-190° C.

### Example 110: 6-benzyl-3-(((1,4-dihydroquinazolin-2-yl)thio)methyl)-2-iodo-5,6-dihydroimidazo[2,1-b]thiazole dihydrochloride

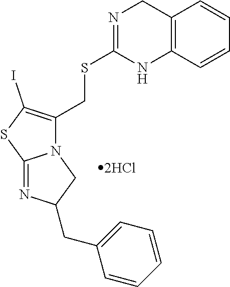 [SMILES: IC1=C(CSC2=NCc3ccccc3N2)N2CC(Cc3ccccc3)N=C2S1]

Example 110 was obtained as a white solid (32 mg, 43%) by centrifugation of the reaction mixture followed by trituration of the solid in EtOH (2×2 mL) and in Et₂O (2×3 mL) and freeze-drying in water.

¹H-NMR (DMSO-d₆, 400 MHz) δ:3.07-3.18 (m, 2H, CH₂-Ph); 4.35 (dd, J 10.6, 7.0 Hz, 1H, N—CHₐH_{b}); 4.56-4.61 (m, 3H, N—CHₐH_{b}+N-CH₂—Ar); 4.72 (bs, 2H, S—CH₂); 5.00-5.08 (m, 1H, N—CH); 7.20-7.38 (m, 9H, 9 Ar); 10.08 (bs, 1H, HCl salt); 11.30 (bs, 1H, HCl salt); 12.99 (bs, 1H, NH). M/Z (M+H)⁺: 519.1. Mp: 177-182° C.

### Example 111: (S)-6-(3-chlorobenzyl)-3-(((1,4-dihydroquinazolin-2-yl)thio)methyl)-5,6-dihydroimidazo[2,1-b]thiazole dihydrochloride

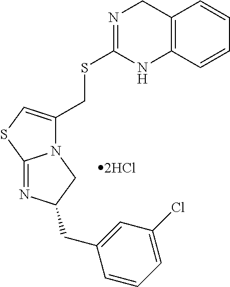 [SMILES: Clc1cccc(C[C@H]2CN3C(CSC4=NCc5ccccc5N4)=CSC3=N2)c1]

Crude example 111 was obtained by concentration to dryness of the reaction mixture followed by hydrolysis with water (15 mL) and washing with EtOAc (2×10 mL). 1 N aqueous HCl was added to the resulting aqueous layer thas was then freeze-dried to afford a white solid (86 mg, 74%).

¹H-NMR (DMSO-d₆, 400 MHz) δ: 3.12 (d, J 6.7 Hz, 2H, CH₂—Ar); 4.30 (dd, J10.9, 6.9 Hz, 1H, N—CHₐH_{b}); 4.54 (t, J 10.5 Hz, 1H, N—CHₐH_{b}); 4.65 (bs, 2H, N—CH₂); 4.74-4.96 (m, 2H, S—CH₂); 5.00-5.08 (m, 1H, N—CH); 7.00 (s, 1H, S—CH); 7.18-7.26 (m, 2H, 2 Ar); 7.28-7.41 (m, 5H, 5 Ar); 7.44 (s, 1H, Ar); 10.05 (bs, 1H, HCl salt); 11.32 (bs, 1H, HCl salt); 12.95 (bs, 1H, NH). M/Z (M[³⁵Cl]+H)⁺: 427.1. Mp: 185-200° C.

### Example 112: 3-(((1,4-dihydroquinazolin-2-yl)thio)methyl)-6-(3-methylbenzyl)-5,6-dihydroimidazo[2,1-b]thiazole dihydrochloride

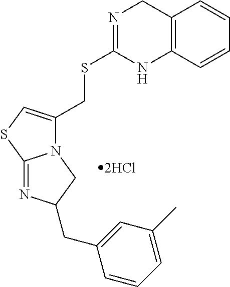 [SMILES: Cc1cccc(CC2CN3C(CSC4=NCc5ccccc5N4)=CSC3=N2)c1]

Crude example 112 was obtained by concentration to dryness of the reaction mixture followed by hydrolysis with water (15 mL) and washing with EtOAc (2×10 mL). 1 N aqueous HCl was added to the resulting aqueous layer thas was freeze-dried to afford a white solid (98 mg, 83%).

¹H-NMR (DMSO-d₆, 400 MHz) δ: 2.30 (s, 3H, CH₃); 3.01-3.11 (m, 2H, CH₂—Ar); 4.28 (dd, J10.7, 7.1 Hz, 1H, N—CHₐH_{b}); 4.53 (t, J 10.5 Hz, 1H, N—CHₐH_{b}); 4.65 (s, 2H, N—CH₂); 4.74-4.94 (m, 2H, S—CH₂); 4.97-5.05 (m, 1H, N—CH); 6.99 (s, 1H, S—CH); 7.07-7.14 (m, 3H, 3 Ar); 7.18-7.35 (m, 5H, 5 Ar); 10.03 (s, 1H, HCl salt); 11.28 (bs, 1H, HCl salt); 12.94 (bs, 1H, NH). M/Z (M+H)⁺: 407.1. Mp: 136-145° C.

### Example 113: 6-benzyl-3-(((4-methyl-1,4-dihydroquinazolin-2-yl)thio)methyl)-5,6-dihydroimidazo[2,1-b]thiazole dihydrochloride

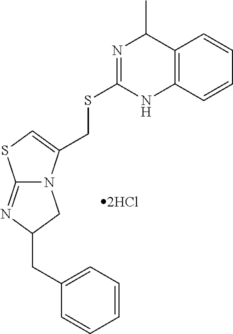 [SMILES: CC1N=C(SCC2=CSC3=NC(Cc4ccccc4)CN23)Nc2ccccc21]

Crude example 113 was obtained by concentration to dryness of the reaction mixture followed by hydrolysis with water (10 mL) and washing with EtOAc (2×10 mL). The resulting aqueous layer was freeze-dried, purified by preparative HPLC (column A, H₂O+0.1% HCOOH/MeCN+0.1% HCOOH 95:5 to 55:45) and freeze-dried with 1 N aqueous HCl (5 equiv) to obtain a white solid (72 mg, 60%).

¹H-NMR (DMSO-d₆, 400 MHz) δ: 1.37 (d, J 4.4 Hz, 1.5H, one diastereoisomer of CH—CH₃); 1.39 (d, J 4.4 Hz, 1.5H, other diastereoisomer of CH—CH₃); 3.07-3.15 (m, 2H, CH₂-Ph); 4.29 (dd, J 10.8, 7.2 Hz, 0.5H, one diastereoisomer of N—CHₐH_{b}); 4.35 (dd, J 10.8, 7.2 Hz, 0.5H, other diastereoisomer of N—CHₐH_{b}); 4.51-4.66 (m, 2H, N—CHₐH_{b}+N-CH); 4.97-5.08 (m, 2H, S—CH₂); 5.16 (d, J 15.4 Hz, 0.5H, one diastereoisomer of N—CH—CH₃); 5.23 (d, J 15.4 Hz, 0.5H, other diastereoisomer of N—CH—CH₃); 7.00-7.08 (m, 1H, S—CH); 7.18-7.38 (m, 9H, 9 Ar); 10.11 (bs, 1H, HCl salt); 11.54-11.59 (m, 1H, HCl salt); 13.00-13.03 (m, 1H, NH). M/Z (M+H)⁺: 407.3. Mp: 80-88° C.

### Example 114: 6-benzyl-3-(((6-chloro-1,4-dihydroquinazolin-2-yl)thio)methyl)-5,6-dihydroimidazo[2,1-b]thiazole dihydrochloride

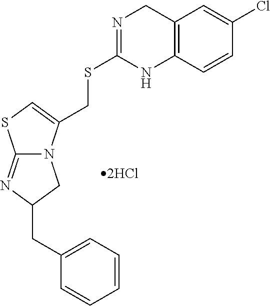 [SMILES: Clc1ccc2c(c1)CN=C(SCC1=CSC3=NC(Cc4ccccc4)CN13)N2]

Crude example 114 was obtained by concentration to dryness of the reaction mixture followed by purification by preparative HPLC (column B, H₂O+0.1% HCOOH/MeCN+0.1% HCOOH 95:5 to 55:45) and freeze-drying with 1 N aqueous HCl (5.0 equiv). The resulting solid was dissolved in water (20 mL) and washed with EtOAc (2×20 mL). The aqueous layer was freeze-dried, purified by preparative HPLC (column A, H₂O+0.1% HCOOH/MeCN+0.1% HCOOH 95:5 to 55:45) and freeze-dried with 1 N aqueous HCl (5 equiv). The residue was further purified by preparative HPLC (column B, H₂O+0.1% HCOOH/MeCN+0.1% HCOOH 95:5 to 55:45) and freeze-dried with 1 N aqueous HCl (5 equiv) to obtain a white solid (23 mg, 18%).

¹H-NMR (DMSO-d₆, 400 MHz) δ: 3.06-3.15 (m, 2H, CH₂-Ph); 4.28 (dd, J 10.8, 7.2 Hz, 1H, N—CHₐH_{b}); 4.52 (dd, J 10.8, 10.0 Hz, 1H, N—CHₐH_{b}); 4.64 (bs, 2H, N—CH₂); 4.79-4.91 (m, 2H, S—CH₂); 4.99-5.06 (m, 1H, N—CH); 6.99 (bs, 1H, S—CH); 7.24-7.28 (m, 1H, Ar); 7.32-7.41 (m, 7H, 7 Ar); 10.03 (s, 1H, HCl salt); 11.39 (bs, 1H, HCl salt); 13.13 (bs, 1H, NH). M/Z (M[³⁵Cl]+H)⁺: 427.2. Mp: 156-184° C.

### Example 115: 2-((2-(indolin-1-yl)ethyl)thio)-1,4-dihydroquinazoline hydrochloride

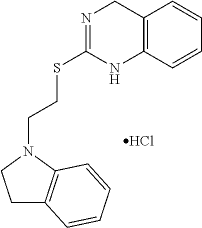 [SMILES: c1ccc2c(c1)CN=C(SCCN1CCc3ccccc31)N2 ; Cl]

Crude example 115 was obtained by concentration to dryness of the reaction mixture. The solid was then passed through an ISOLUTE® SCX-2 cartridge (DCM and MeOH, then NH₃ 7 M in MeOH) and concentrated to dryness. To a solution of the resulting oil in DCM was added HCl in Et₂O and, after evaporation to dryness, the residue was purified by flash chromatography (CyHex 100% to CyHex/EtOAc 50:50 then DCM 100% to DCM/MeOH 90:10) and freeze-dried with 1 N aqueous HCl (5 equiv) to obtain a grey solid (131 mg, 14% over 2 steps).

¹H-NMR (DMSO-d₆, 400 MHz) δ: 2.76 (t, J8.2 Hz, 2H, CH₂—Ar); 3.37-3.42 (m, 4H, 2 N—CH₂); 3.70 (t, J6.2 Hz, 2H, S—CH₂); 4.54 (bs, 2H, N—CH₂); 6.56-6.60 (m, 2H, 2Ar); 6.95-7.01 (m, 2H, 2Ar); 7.13 (d, J7.8 Hz, 1H, Ar); 7.17-7.23 (m, 2H, 2 Ar); 7.27-7.31 (m, 1H, Ar); 10.76 (bs, 1H, HCl salt); 12.49 (bs, 1H, HCl salt); NH signal not observed. M/Z (M+H)⁺: 310.1. Mp: 82-88° C.

### Example 116: 4-chloro-2-(((1,4-dihydroquinazolin-2-yl)thio)methyl)thieno[3,2-c]pyridine dihydrochloride

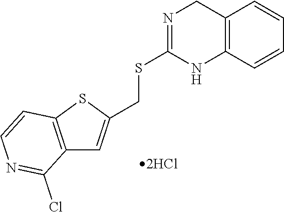 [SMILES: Clc1nccc2sc(CSC3=NCc4ccccc4N3)cc12]

Crude example 116 was obtained by centrifugation of the reaction mixture. The solid was triturated in EtOH (4×2 mL). The resulting solid was triturated in hot MeOH (6×2 mL) and the combined methanolic supernatants were concentrated to dryness and passed through an ISOLUTE® SCX-2 cartridge (MeOH, then NH₃2 N in MeOH). To a solution of the resulting oily residue in DCM was added HCl in Et₂O and, after evaporation to dryness the resulting crude yellow solid was triturated in water (5 mL+2×2 mL) and freeze-dried in water to afford a beige solid (43 mg, 34%).

¹H-NMR (DMSO-d₆, 400 MHz) δ: 4.66 (s, 2H, N—CH₂); 5.18 (s, 2H, S—CH₂); 7.18-7.25 (m, 3H, 3 Ar); 7.31-7.35 (m, 1H, Ar); 7.63 (s, 1H, Ar); 8.06 (dd, J 5.6, 0.6 Hz, 1H, Ar); 8.22 (d, J 5.6 Hz, 1H, Ar); 10.99 (bs, 1H, HCl salt); 12.69 (bs, 1H, HCl salt), NH signal not observed. M/Z (M[³⁵Cl+H]⁺: 346.0. Mp>240-245° C.

### Example 117: 6-benzyl-3-(((5-fluoro-1,4-dihydroquinazolin-2-yl)thio)methyl)-5,6-dihydroimidazo[2,1-b]thiazole dihydrochloride

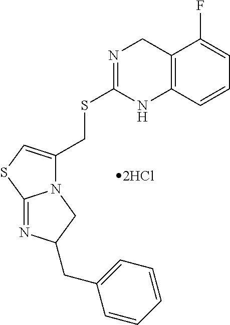 [SMILES: Fc1cccc2c1CN=C(SCC1=CSC3=NC(Cc4ccccc4)CN13)N2]

Crude example 117 was obtained by hydrolysis of the reaction mixture with water (20 mL) and 1 N aqueous HCl (2 mL), then washing with EtOAc (2×30 mL), followed by freeze-drying of the resulting aqueous layer. The residue was purified by preparative HPLC (column B, H₂O+0.1% HCOOH/MeCN+0.1% HCOOH 95:5 to 55:45) and freeze-dried with 1 N aqeous HCl (5 equiv). The residue was dissolved in EtOH (4 mL) and filtered. The filtrate was concentrated to dryness, dissolved EtOH (1 mL) and precipitated with Et₂O (4 mL). The solid isolated by centrifugation was washed with Et₂O (2 mL), purified by Sephadex-LH20 (MeOH 100%) and freeze-dried in H₂O (3 mL) and MeCN (0.5 mL) to obtain a white solid (33 mg, 27%).

¹H-NMR (DMSO-d₆, 400 MHz) δ: 3.05-3.15 (m, 2H, CH₂-Ph); 4.27 (dd, J 10.4, 7.2 Hz, 1H, N—CHₐH_{b}); 4.50 (t, J 10.4 Hz, 1H, N—CHₐH_{b}); 4.65 (bs, 2H, N—CH₂); 4.58-4.87 (m, 2H, S—CH₂); 4.98-5.06 (m, 1H, N—CH); 6.97 (bs, 1H, S—CH); 7.01-7.14 (m, 2H, 2 Ar); 7.24-7.37 (m, 6H, 6 Ar); 10.03 (s, 1H, HCl salt); 11.43 (bs, 1H, HCl salt); 13.14 (bs, 1H, NH). M/Z (M+H)⁺: 411.1. Mp: 147-151° C.

### Example 118: 6-benzyl-3-(((5-chloro-1,4-dihydroquinazolin-2-yl)thio)methyl)-5,6-dihydroimidazo[2,1-b]thiazole dihydrochloride

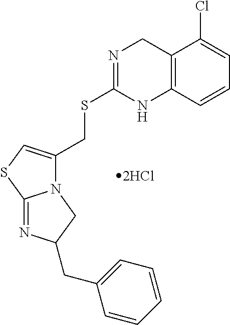 [SMILES: Clc1cccc2c1CN=C(SCC1=CSC3=NC(Cc4ccccc4)CN13)N2]

Crude example 118 was obtained by hydrolysis of the reaction mixture with water (20 mL) and 1 N aqueous HCl (2 mL) then washing with EtOAc (2×30 mL), followed by freeze-drying of the resulting aqueous layer. The residue was purified by preparative HPLC (column B, H₂O+0.1% HCOOH/MeCN+0.1% HCOOH 95:5 to 55:45) and freeze-dried with 1 N aqeous HCl (5 equiv). The residue was dissolved in EtOH (4 mL) and filtered. The filtrate was concentrated to dryness, dissolved EtOH (1 mL) and precipitated with Et₂O (4 mL). The solid isolated by centrifugation was washed with Et₂O (2 mL), purified by Sephadex-LH20 (MeOH 100%) and freeze-dried in H₂O (3 mL) and MeCN (0.5 mL) to obtain a white solid (31 mg, 27%).

¹H-NMR (DMSO-d₆, 400 MHz) δ: 3.05-3.15 (m, 2H, CH₂-Ph); 4.28 (dd, J10.8, 7.2 Hz, 1H, N—CHₐH_{b}); 4.48-4.53 (m, 1H, N—CHₐH_{b}); 4.64 (bs, 2H, N—CH₂); 4.68-4.91 (m, 2H, S—CH₂); 4.98-5.06 (m, 1H, N—CH); 6.99 (bs, 1H, S—CH); 7.24- 7.37 (m, 8H, 8 Ar); 10.03 (s, 1H, HCl salt); 11.47 (bs, 1H, HCl salt); 13.07 (bs, 1H, NH). M/Z (M[³⁵Cl]+H)⁺: 427.1. Mp: 151-158° C.

### Example 119: 6-benzyl-3-(((7-bromo-1,4-dihydroquinazolin-2-yl)thio)methyl)-5,6-dihydroimidazo[2,1-b]thiazole dihydrochloride

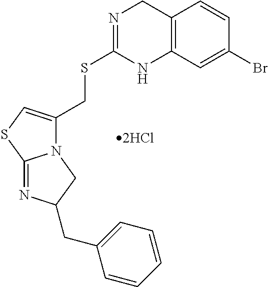 [SMILES: Brc1ccc2c(c1)NC(SCC1=CSC3=NC(Cc4ccccc4)CN13)=NC2]

Example 119 was isolated as a white solid (48 mg, 35%) by filtration the reaction mixture, followed by purification of the solid by preparative HPLC (column B, H₂O+0.1% HCOOH/MeCN+0.1% HCOOH 95/5 to 55:45) and freeze-drying in 1 N aqueous HCl (5 equiv).

¹H-NMR (DMSO-d₆, 400 MHz) δ: 3.05-3.15 (m, 2H, CH₂-Ph); 4.28 (dd, J10.8, 7.2 Hz, 1H, N—CHₐH_{b}); 4.49-4.54 (m, 1H, N—CHₐH_{b}); 4.60 (bs, 2H, N—CH₂); 4.70-4.94 (m, 2H, S—CH₂); 4.98-5.06 (m, 1H, N—CH); 6.98 (bs, 1H, S—CH); 7.13- 7.19 (m, 1H, Ar); 7.24-7.54 (m, 7H, 7 Ar); 10.02 (s, 1H, HCl salt); 11.43 (bs, 1H, HCl salt); 13.21 (bs, 1H, NH). M/Z (M[⁷⁹Br]+H)⁺: 471.0. Mp: 158-166° C.

### Example 120: 3-(((1,4-dihydroquinazolin-2-yl)thio)methyl)-6-phenyl-5,6-dihydroimidazo[2,1-b]thiazole dihydrochloride

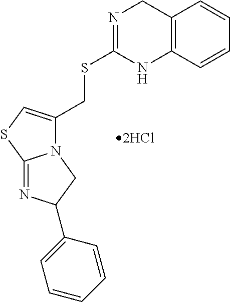 [SMILES: C1=C(CSC2=NCc3ccccc3N2)N2CC(c3ccccc3)N=C2S1]

Crude example 120 was obtained by concentration to dryness of the reaction mixture, then hydrolysis with water (15 mL) and washing with DCM (3×10 mL), followed by freeze-drying of the resulting aqueous layer. The residue was purified by preparative HPLC (column B, H₂O+0.1% HCOOH/MeCN+0.1% HCOOH 95:5 to 55:45) and freeze-dried with 1 N aqueous HCl (5 equiv).

¹H-NMR (DMSO-d₆, 400 MHz) δ: 4.38 (dd, J 10.8, 8.6 Hz, 1H, N—CHₐH_{b}); 4.67 (s, 2H, N—CH₂—Ar); 4.84-4.94 (m, 2H, S—CH₂); 4.99 (t, J 10.8 Hz, 1H, N—CHₐH_{b}); 5.90 (dd, J 10.8, 8.6 Hz, 1H, N—CH—Ar); 7.12 (s, 1H, S—CH); 7.21-7.35 (m, 4H, 4 Ar); 7.40-7.52 (m, 5H, 5 Ar); 10.64 (bs, 1H, HCl salt); 11.28 (bs, 1H, HCl salt); 12.89 (bs, 1H, NH). M/Z (M+H)⁺: 379.2. Mp: 185-190° C.

### Example 121: 3-(((1,4-dihydroquinazolin-2-yl)thio)methyl)-6-(3-fluorobenzyl)-5,6-dihydroimidazo[2,1-b]thiazole dihydrochloride

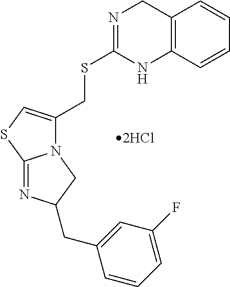 [SMILES: Fc1cccc(CC2CN3C(CSC4=NCc5ccccc5N4)=CSC3=N2)c1]

Crude example 121 was obtained by concentration to dryness of the reaction mixture, then hydrolysis with water (10 mL) and 1 N aqueous HCl (5 mL), then washing with DCM (2×5 mL), followed by freeze-drying of the resulting aqueous layer. The residue was purified by preparative HPLC (column B, H₂O+0.1% HCOOH/MeCN+0.1% HCOOH 95:5 to 55:45) and freeze-dried with 1 N aqueous HCl (5.0 equiv) to afford a white solid (55 mg, 35%).

¹H-NMR (DMSO-d₆, 400 MHz) δ: 3.14 (d, J 6.8 Hz, 2H, CH₂—Ar); 4.31 (dd, J10.9, 6.9 Hz, 1H, N—CHₐH_{b}); 4.54 (t, J 10.5 Hz, 1H, N—CHₐH_{b}); 4.66 (bs, 2H, N—CH₂); 4.82 (d, J 15.3 Hz, 1H, S—CHₐH_{b}); 4.92 (d, J 15.3 Hz, 1H, S—CHₐH_{b}); 5.00-5.08 (m, 1H, N—CH); 7.02 (s, 1H, S—CH); 7.08-7.13 (m, 1H, Ar); 7.18-7.28 (m, 4H, 4Ar); 7.36-7.36 (m, 2H, 2Ar); 7.38-7.43 (m, 1H, Ar); 10.08 (bs, 1H, HCl salt); 11.30 (bs, 1H, HCl salt); 12.95 (bs, 1H, NH). M/Z (M+H)⁺: 411.2.

### Example 122: 3-(((1,4-dihydroquinazolin-2-yl)thio)methyl)-6-(4-methylbenzyl)-5,6-dihydroimidazo[2,1-b]thiazole dihydrochloride

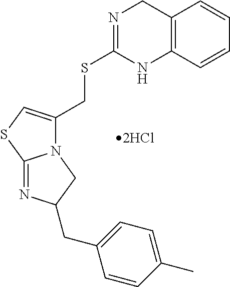 [SMILES: Cc1ccc(CC2CN3C(CSC4=NCc5ccccc5N4)=CSC3=N2)cc1]

Crude example 122 was obtained by concentration to dryness of the reaction mixture, then hydrolysis with water (10 mL) and 1 N aqueous HCl (5 mL), then washing with DCM (2×5 mL), followed by freeze-drying of the resulting aqueous layer. The residue was purified by preparative HPLC (column B, H₂O+0.1% HCOOH/MeCN+0.1% HCOOH 95:5 to 55:45) and freeze-dried with 1 N aqueous HCl (5.0 equiv) to afford a white solid (119 mg, 49%).

¹H-NMR (DMSO-d₆, 400 MHz) δ: 2.26 (s, 3H, CH₃); 3.00-3.11 (m, 2H, CH₂—Ar); 4.26 (dd, J10.6, 7.2 Hz, 1H, N—CHₐH_{b}); 4.51 (t, J 10.6 Hz, 1H, N—CHₐH_{b}); 4.65 (s, 2H, N—CH₂—Ar); 4.79-4.92 (m, 2H, S—CH₂); 4.92-5.04 (m, 1H, N—CH); 7.01 (s, 1H, S—CH); 7.12-7.16 (m, 2H, 2 Ar); 7.20-7.26 (m, 4H, 4 Ar); 7.29-7.36 (m, 2H, 2 Ar); 10.04 (s, 1H, HCl salt); 11.29 (bs, 1H, HCl salt); 12.95 (bs, 1H, NH). M/Z (M+H)⁺: 407.2. Mp: 158-170° C.

### Example 123: 6-(2-chlorobenzyl)-3-(((1,4-dihydroquinazolin-2-yl)thio)methyl)-5,6-dihydroimidazo[2,1-b]thiazole dihydrochloride

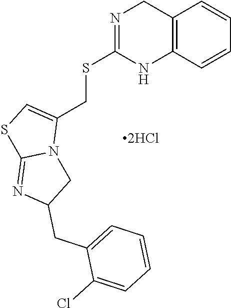 [SMILES: Clc1ccccc1CC1CN2C(CSC3=NCc4ccccc4N3)=CSC2=N1]

Crude example 123 was obtained by concentration to dryness of the reaction mixture, then hydrolysis with water (10 mL) and 1 N aqueous HCl (5 mL), then washing with DCM (2×5 mL), followed by freeze-drying of the resulting aqueous layer. The residue was purified by preparative HPLC (column B, H₂O+0.1% HCOOH/MeCN+0.1% HCOOH 95:5 to 55:45) and freeze-dried with 1 N aqueous HCl (5.0 equiv) to afford a white solid (53 mg, 49%).

¹H-NMR (DMSO-d₆, 400 MHz) δ: 3.23 (dd, J 14.0, 7.2 Hz, 1H, Ar—CHₐH_{b}); 3.31 (dd, J 14.0, 6.9 Hz, 1H, Ar—CHₐH_{b}); 4.33 (dd, J10.8, 7.2 Hz, 1H, N—CHₐH_{b}); 4.59 (t, J10.4 Hz, 1H, N—CHₐH_{b}); 4.66 (s, 2H, N—CH₂—Ar); 4.81-4.97 (m, 2H, S—CH₂); 5.05-5.13 (m, 1H, N—CH); 7.05 (s, 1H, S—CH); 7.23 (bs, 2H, 2 Ar); 7.30-7.40 (m, 4H, 4 Ar); 7.48-7.52 (m, 2H, 2 Ar); 10.18 (s, 1H, HCl salt); 11.32 (bs, 1H, HCl salt); 12.97 (bs, 1H, NH). M/Z (M[³⁵Cl]+H)⁺: 427.2. Mp: 163-177° C.

### Example 124: (R)-3-(((1,4-dihydroquinazolin-2-yl)thio)methyl)-6-(4-methoxybenzyl)-5,6-dihydroimidazo[2,1-b]thiazole dihydrochloride

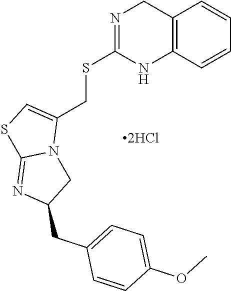 [SMILES: COc1ccc(C[C@@H]2CN3C(CSC4=NCc5ccccc5N4)=CSC3=N2)cc1]

Crude example 124 was obtained by concentration to dryness of the reaction mixture, then hydrolysis with water (10 mL) and 1 N aqueous HCl (5 mL), then washing with DCM (2×5 mL), followed by freeze-drying of the resulting aqueous layer. The residue was purified by preparative HPLC (column B, H₂O+0.1% HCOOH/MeCN+0.1% HCOOH 95:5 to 55:45) and freeze-dried with 1 N aqueous HCl (5.0 equiv) to afford a white solid (65 mg, 51%).

¹H-NMR (DMSO-d₆, 400 MHz) δ: 2.98-3.08 (m, 2H, CH₂—Ar); 3.72 (s, 3H, O—CH₃); 4.26 (dd, J 10.9, 7.1 Hz, 1H, N—CHₐH_{b}); 4.50 (t, J 10.5 Hz, 1H, N—CHₐH_{b}); 4.66 (s, 2H, N—CH₂—Ar); 4.77-4.90 (m, 2H, S—CH₂); 4.93-5.01 (m, 1H, N—CH); 6.89-6.92 (m, 2H, 2 Ar); 6.99 (bs, 1H, S—CH); 7.22-7.29 (m, 4H, 4 Ar); 7.29-7.35 (m, 2H, 2 Ar); 10.02 (bs, 1H, HCl salt); 11.26 (bs, 1H, HCl salt); 12.92 (bs, 1H, NH). M/Z (M+H)⁺: 423.2. Mp: 150-164° C.

### Example 125: 2-((2-(3,3-difluoropyrrolidin-1-yl)ethyl)thio)-1,4-dihydroquinazoline dihydrochloride

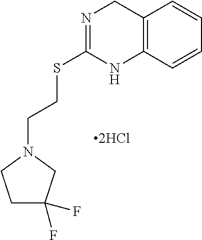 [SMILES: FC1(F)CCN(CCSC2=NCc3ccccc3N2)C1]

Crude example 125 was obtained by filtration of the reaction mixture followed by concentration to dryness of the filtrate. The resulting oil was passed through an ISOLUTE® SCX-2 cartridge (MeOH/DCM, then NH₃ 2 N in MeOH). To a solution of the resulting residue in DCM (2.0 mL) was added HCl in Et₂O (2.0 equiv) and, after evaporation to dryness, the resulting crude orange solid was purified by flash chromatography (DCM 100% to DCM/MeOH 90:10) and freeze-dried with 1 N aqueous HCl (5.0 equiv) to afford a beige hygroscopic solid (190 mg, 39% over 2 steps). ¹H-NMR (DMSO-d₆, 400 MHz) δ: 2.37-2.46 (m, 2H, CH₂); 3.13-3.36 (m, 4H, 2 CH₂); 3.64-3.77 (m, 4H, 2 CH₂); 4.71 (s, 2H, N—CH₂—Ar); 7.20-7.27 (m, 3H, 3 Ar); 7.30-7.36 (m, 1H, Ar); 11.05 (bs, 1H, HCl salt); 12.60 (bs, 1H, NH); HCl salt signal not observed. M/Z (M+H)⁺: 298.1.

### Example 126: 3-(((1,4-dihydroquinazolin-2-yl)thio)methyl)-6-phenethyl-5,6-dihydroimidazo[2,1-b]thiazole dihydrochloride

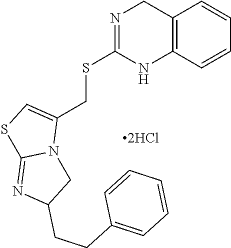 [SMILES: C1=C(CSC2=NCc3ccccc3N2)N2CC(CCc3ccccc3)N=C2S1]

Crude example 126 was obtained by concentration to dryness of the reaction mixture, then hydrolysis with water (10 mL) and 1 N aqueous HCl (5 mL), then washing with DCM (3×5 mL), followed by freeze-drying of the resulting aqueous layer. The residue was purified twice by preparative HPLC (column B, H₂O+0.1% HCOOH/MeCN+0.1% HCOOH 95:5 to 55:45, then column A, H₂O+0.1% HCOOH/MeCN+0.1% HCOOH 95:5 to 55:45) and freeze-dried with 1 N aqueous HCl (5.0 equiv) to afford a white solid (57 mg, 51%).

¹H-NMR (DMSO-d₆, 400 MHz) δ: 1.98-2.13 (m, 2H, N—CH—CH₂); 2.65-2.76 (m, 2H, CH₂-Ph); 4.30 (dd, J10.2, 7.2 Hz, 1H, N—CHₐH_{b}); 4.60-4.81 (m, 4H, N—CHₐH_{b}+N-CH₂—Ar+N-CH); 4.81-4.96 (m, 2H, S—CH₂); 7.02 (s, 1H, S—CH); 7.19-7.34 (m, 9H, 9 Ar); 10.45 (s, 1H, HCl salt); 11.28 (bs, 1H, HCl salt); 12.93 (bs, 1H, NH). M/Z (M+H)⁺: 407.1. Mp: 136-142° C.

### Example 127: 2-((2-(3-methoxypyrrolidin-1-yl)ethyl)thio)-1,4-dihydroquinazoline dihydrochloride

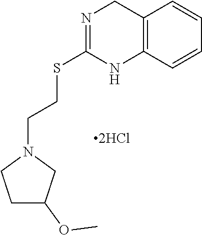 [SMILES: COC1CCN(CCSC2=NCc3ccccc3N2)C1]

Crude example 127 was obtained by filtration of the reaction mixture followed by concentration to dryness of the filtrate. The resulting oil was passed through an ISOLUTE® SCX-2 cartridge (MeOH/DCM, then NH₃ 2 N in MeOH). To a solution of the resulting residue in DCM (2.0 mL) was added HCl in Et₂O (2.0 equiv) and, after evaporation to dryness, the resulting crude orange solid was purified by flash chromatography (KPNH, DCM 100% to DCM/MeOH 90:10). The residue was dissolved in 1 N aqueous HCl, washed with DCM (2×10 mL). The resulting aqueous layer was freeze-dried, purified by preparative HPLC (column B, H₂O+0.1% HCOOH/MeCN+0.1% HCOOH 95:5 to 55:45) and freeze-dried with 1 N aqueous HCl (5.0 equiv) to afford a white hygroscopic solid (50 mg, 17% over 3 steps).

¹H-NMR (DMSO-d₆, 400 MHz) δ: 1.89-2.29 (m, 2H, CH₂); 3.12-3.25 (m, 5H, O-CH₃+N-CH₂); 3.35-3.48 (m, 4H, 2 N—CH₂); 3.77-3.88 (m, 2H, S—CH₂); 4.13 (bs, 1H, O—CH); 4.73 (s, 2H, N—CH₂—Ar); 7.21-7.28 (m, 3H, 3 Ar); 7.30-7.37 (m, 1H, Ar); 10.73-11.40 (m, 2H, 2 HCl salts); 12.70 (bs, 1H, NH). M/Z (M+H)⁺: 292.1.

### Example 128: 2-((2-(2-phenylpyrrolidin-1-yl)ethyl)thio)-1,4-dihydroquinazoline dihydrochloride

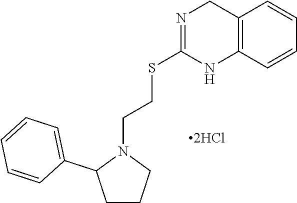 [SMILES: c1ccc(C2CCCN2CCSC2=NCc3ccccc3N2)cc1]

Crude example 128 was obtained by concentration to dryness of the reaction mixture. The resulting oil was passed through an ISOLUTE® SCX-2 cartridge (MeOH/DCM, then NH₃ 2 N in MeOH). To a solution of the resulting residue in DCM (2.0 mL) was added HCl in Et₂O (2.0 equiv) and, after evaporation to dryness, the resulting crude orange oil was purified by flash chromatography (DCM 100% to DCM/MeOH 90:10, then DCM/[MeOH+1% NH₄OH 28% aq.]90:10 to DCM/[MeOH+1% NH₄OH 28% aq.] 80:20) then by preparative HPLC (column B, H₂O+0.1% HCOOH/MeCN+0.1% HCOOH 95:5 to 55:45) and freeze-dried with 1 N aqueous HCl (5.0 equiv) to afford a white solid (25 mg, 10% over 3 steps).

¹H-NMR (DMSO-d₆, 400 MHz) δ: 2.06-2.24 (m, 3H, CH₂+CHₐH_{b}); 2.39-2.45 (m, 1H, CHₐH_{b}); 3.11-3.23 (m, 1H, N—CHₐH_{b}); 3.25-3.34 (m, 1H, N—CHₐH_{b}); 3.57-3.69 (m, 2H, N—CH₂); 3.79-3.99 (m, 2H, S—CH₂); 4.37-4.53 (m, 1H, CH—Ar); 4.66 (s, 2H, N—CH₂—Ar); 7.14-7.27 (m, 3H, 3 Ar); 7.29-7.36 (m, 1H, Ar); 7.39-7.48 (m, 3H, 3 Ar); 7.62-7.77 (m, 2H, 2 Ar); 10.93 (bs, 1H, HCl salt); 11.20 (bs, 1H, HCl salt); 12.57 (bs, 1H, NH). M/Z (M+H)⁺: 338.2. Mp: 94-108° C.

### Example 129: 2-((2-(pyrrolidin-1-yl)propyl)thio)-1,4-dihydroquinazoline dihydrochloride

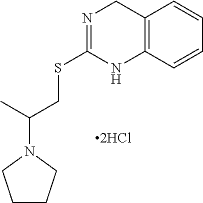 [SMILES: CC(CSC1=NCc2ccccc2N1)N1CCCC1]

Crude example 129 was obtained after filtration of the reaction mixture followed by concentration to dryness of the filtrate. The resulting oil was passed through an ISOLUTE® SCX-2 cartridge (MeOH/DCM, then NH₃ 2 N in MeOH). To a solution of the resulting residue in DCM (2.0 mL) was added HCl in Et₂O (2.0 equiv) and, after evaporation to dryness, the resulting crude orange oil was purified by flash chromatography (KPNH, CyHex 100% to CyHex/EtOAc 0:100). The residue was dissolved in 1 N aqueous HCl, washed with DCM (2×10 mL). The resulting aqueous layer was freeze-dried, purified by preparative HPLC (column B, H₂O+0.1% HCOOH/MeCN+0.1% HCOOH 95:5 to 55:45) and freeze-dried with 1 N aqueous HCl (5.0 equiv) to afford a white hygroscopic solid (61 mg, 13% over 2 steps).

¹H-NMR (DMSO-d₆, 400 MHz) δ: 1.42 (d, J6.4 Hz, 3H, CH₃); 1.87-2.03 (m, 4H, 2 CH₂); 305-3.23 (m, 2H, N—CH₂); 3.50-3.59 (m, 3H, S—CH₂+N-CHₐH_{b}); 3.66-3.74 (m, 1H, N—CHₐH_{b}); 4.09-4.16 (m, 1H, N—CH); 4.68-4.77 (m, 2H, N—CH₂); 7.21-7.27 (m, 3H, 3 Ar); 7.30-7.36 (m, 1H, Ar); 11.10 (bs, 1H, HCl salt); 11.28 (bs, 1H, HCl salt); 12.75 (bs, 1H, NH). M/Z (M+H)⁺: 276.1.

### Example 130: 2-((2-(2-methylpyrrolidin-1-yl)ethyl)thio)-1,4-dihydroquinazoline dihydrochloride

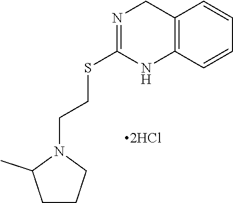 [SMILES: CC1CCCN1CCSC1=NCc2ccccc2N1]

Crude example 130 was obtained after concentration to dryness of the reaction mixture. The resulting oil was passed through an ISOLUTE® SCX-2 cartridge (MeOH/DCM, then NH₃ 2 N in MeOH). To a solution of the resulting residue in DCM (2.0 mL) was added HCl in Et₂O (2.0 equiv) and, after evaporation to dryness, the resulting crude brown oil was purified by flash chromatography (KPNH, CyHex 100% to CyHex/EtOAc 0:100). The residue was dissolved in 1 N aqueous HCl, washed with DCM (2×10 mL). The resulting aqueous layer was freeze-dried, purified by preparative HPLC (column B, H₂O+0.1% HCOOH/MeCN+0.1% HCOOH 95:5 to 55:45) and freeze-dried with 1 N aqueous HCl (5.0 equiv) to afford a white hygroscopic solid (33 mg, 8% over 4 steps).

¹H-NMR (DMSO-d₆, 300 MHz) δ: 1.41 (d, J6.3 Hz, 3H, CH₃); 1.56-1.69 (m, 1H, CHₐH_{b}); 1.89-2.03 (m, 2H, CH₂); 2.14-2.23 (m, 1H, CHₐH_{b}); 3.12-3.25 (m, 1H, N—CH); 3.29-3.51 (m, 3H, N—CH₂+N-CHₐH_{b}); 3.70-3.96 (m, 3H, N—CHₐH_{b}+S-CH₂); 4.73 (s, 2H, N—CH₂—Ar); 7.20-7.26 (m, 3H, 3 Ar); 7.30-7.37 (m, 1H, Ar); 10.77 (bs, 1H, HCl salt); 10.98 (bs, 1H, HCl salt); 12.65 (bs, 1H, NH). M/Z (M+H)⁺: 276.1.

### Example 131: 5-methyl-5-phenyl-2-((2-(pyrrolidin-1-yl)ethyl)thio)-4,5-dihydro-1H-imidazole dihydrochloride

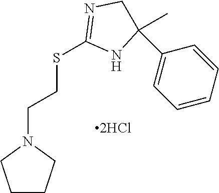 [SMILES: CC1(c2ccccc2)CN=C(SCCN2CCCC2)N1]

Crude example 131 was obtained concentration to dryness of the reaction mixture. The resulting oil was passed through an ISOLUTE® SCX-2 cartridge (MeOH, then NH₃2 N in MeOH). To a solution of the resulting residue in DCM (2.0 mL) was added HCl in Et₂O (2.0 equiv) and, after evaporation to dryness, the resulting beige solid was purified by flash chromatography (KPNH, CyHex 100% to CyHex/EtOAc 0:100 then DCM 100% to DCM/MeOH 95:5) and freeze-dried with 1 N aqueous HCl (5.0 equiv) to afford a white hygroscopic solid (68 mg, 67%).

¹H-NMR (DMSO-d₆, 400 MHz) δ: 1.73 (s, 3H, CH₃); 1.83-1.94 (m, 2H, CH₂); 1.96-2.05 (m, 2H, CH₂); 3.04-3.15 (m, 2H, N—CH₂); 3.48-3.61 (m, 4H, 2 N—CH₂); 3.74-3.80 (m, 2H, S—CH₂); 3.91 (d, J11.2 Hz, 1H, N—CHₐH_{b}); 4.06 (d, J11.2 Hz, 1H, N—CHₐH_{b}); 7.32-7.39 (m, 1H, Ar); 7.41-7.50 (m, 4H, 4 Ar); 10.92 (bs, 1H, HCl salt); 11.09 (bs, 1H, HCl salt); 11.43 (bs, 1H, NH). M/Z (M+H)⁺: 290.1.

### Example 132: 2-((2-(1,1-difluoro-5-azaspiro[2.4]heptan-5-yl)ethyl)thio)-3,4-dihydroquinazoline

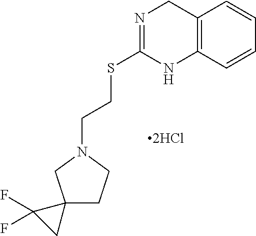 [SMILES: FC1(F)CC12CCN(CCSC1=NCc3ccccc3N1)C2]

Crude example 132 was obtained by concentration to dryness of the reaction mixture. The residue was passed through an ISOLUTE® SCX-2 cartridge (MeOH, then NH₃2 N in MeOH). To a solution of the resulting residue in DCM (2.0 mL) was added HCl in Et₂O (2.0 equiv) and, after evaporation to dryness, the resulting crude brown oil was dissolved in water (20 mL) and washed with DCM (2×10 mL). The aqueous layer was freeze-dried, then purified by preparative HPLC (column B, H₂O+0.1% HCOOH/MeCN+0.1% HCOOH 95:5 to 55:45) and freeze-dried with 1 N aqueous HCl (5.0 equiv) to afford a yellow solid (27 mg, 4% over 4 steps).

¹H-NMR (DMSO-d₆, 400 MHz) δ: 1.70-1.80 (m, 2H, CH₂); 2.06-2.16 (m, 1H, CHₐH_{b}); 2.18-2.27 (m, 1H, CHₐH_{b}); 3.47- 3.67 (m, 5H, 2 N-CH₂+N-CHₐH_{b}); 3.70-3.85 (m, 3H, S—CH₂+N-CHₐH_{b}); 4.73 (s, 2H, N—CH₂—Ar); 7.17-7.22 (m, 1H, Ar); 7.23-7.27 (m, 2H, 2 Ar); 7.30-7.36 (m, 1H, Ar); 10.92 (bs, 1H, HCl salt); 11.33 (bs, 0.5H, one rotamer of HCl salt); 11.70 (bs, 0.5H, other rotamer of HCl salt); 12.59 (bs, 1H, NH). M/Z (M+H)⁺: 324.1.

### Example 133: 2-((2-((1R,5S)-8-azabicyclo[3.2.1]octan-8-yl)ethyl)thio)-3,4-dihydroquinazoline dihydrochloride

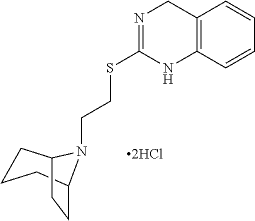 [SMILES: c1ccc2c(c1)CN=C(SCCN1C3CCCC1CC3)N2]

Crude example 133 was obtained by concentration to dryness of the reaction mixture. The residue was passed through an ISOLUTE® SCX-2 cartridge (MeOH, then NH₃2 N in MeOH). To a solution of the resulting residue in DCM (2.0 mL) was added HCl in Et₂O (2.0 equiv) and, after evaporation to dryness, the resulting crude brown oil was dissolved in water (20 mL) and washed with DCM (2×10 mL). The aqueous layer was freeze-dried, then purified by preparative HPLC (column B, H₂O+0.1% HCOOH/MeCN+0.1% HCOOH 95:5 to 55:45) and freeze-dried with 1 N aqueous HCl (5.0 equiv) to afford a white solid (127 mg, 22% over 4 steps).

¹H-NMR (DMSO-d₆, 400 MHz) δ: 1.48-1.56 (m, 1H, CHₐH_{b}); 1.58-1.72 (m, 3H, CH₂+CHₐH_{b}); 1.85-1.91 (m, 2H, CH₂); 2.06-2.19 (m, 4H, 2 CH₂); 3.26-3.32 (m, 2H, N—CH₂); 3.82-3.94 (m, 2H, S—CH₂); 4.03-4.10 (m, 2H, 2 N—CH); 4.73 (s, 2H, N—CH₂—Ar); 7.19-7.26 (m, 3H, 3 Ar); 7.29-7.35 (m, 1H, Ar); 10.48 (bs, 1H, HCl salt); 10.92 (bs, 1H, HCl salt); 12.57 (bs, 1H, NH). M/Z (M+H)⁺: 302.1. Mp: 52-70° C.

### Example 134: 6,7,8-triiodo-2-((2-(pyrrolidin-1-yl)ethyl)thio)-1,4-dihydroquinazoline dihydrochloride

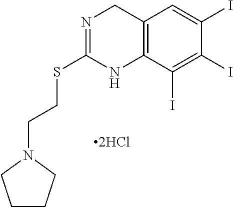 [SMILES: Ic1cc2c(c(I)c1I)NC(SCCN1CCCC1)=NC2]

Crude example 134 was obtained by concentration to dryness of the reaction mixture with a Genevac centrifugal evaporator. The residue was passed through an ISOLUTE® SCX-2 cartridge (MeOH, then NH₃ 2 N in MeOH). To a solution of the resulting crude in DCM and MeOH (2.0 mL) was added HCl in Et₂O (6 mL) and after evaporation to dryness the resulting solid was purified by flash chromatography (DCM 100% to DCM/MeOH 84:16). The obtained orange solid was triturated in MeOH (3×2 mL) and diethyl ether (2×2 mL) to afford a white solid (130 mg, 45%). ¹H-NMR (DMSO-d₆, 400 MHz) δ: 1.90-2.00 (m, 4H, 2 CH₂); 3.09 (bs, 2H, N—CH₂); 3.42-3.45 (m, 2H, N—CH₂); 3.50-3.64 (bs, 4H, N—CH₂+S-CH₂); 4.32 (s, 2H, N—CH₂—Ar); 7.65 (s, 1H, Ar); 8.28 (s, 1H, HCl salt); 10.34 (s, 1H, HCl salt); NH signal not observed. M/Z (M+H)⁺: 639.8. Mp: 190-195° C.

### Example 135: 1-(2-((1,4-dihydroquinazolin-2-yl)thio)ethyl)pyrrolidin-2-one hydrochloride

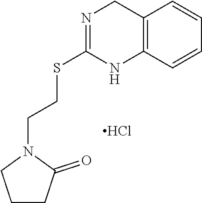 [SMILES: O=C1CCCN1CCSC1=NCc2ccccc2N1 ; Cl]

Crude example 135 was obtained by concentration to dryness of the reaction mixture. The residue was passed through an ISOLUTE® SCX-2 cartridge (MeOH, then NH₃2 N in MeOH). To a solution of the resulting residue in DCM (2.0 mL) was added HCl in Et₂O (2.0 equiv) and, after evaporation to dryness, the resulting crude yellow oil was purified by flash chromatography (KPNH, CyHex 100% to CyHex/EtOAc 0:100) and freeze-dried with 1 N aqueous HCl (5.0 equiv). The resulting white solid was purified by preparative HPLC (column B, H₂O+0.1% HCOOH/MeCN+0.1% HCOOH 95:5 to 55:45) and freeze-dried with 1 N aqueous HCl (5.0 equiv) to afford a white hygroscopic solid (107 mg, 34%).

¹H-NMR (DMSO-d₆, 400 MHz) δ: 1.83-1.91 (m, 2H, CH₂); 2.16 (t, J8.1 Hz, 2H, CO—CH₂); 3.41 (t, J6.9 Hz, 2H, N—CH₂); 3.52 (t, J 6.1 Hz, 2H, N—CH₂); 3.63 (t, J 6.1 Hz, 2H, S—CH₂); 4.69 (s, 2H, N—CH₂—Ar); 7.20-7.26 (m, 3H, 3 Ar); 7.30-7.36 (m, 1H, Ar); 10.82 (bs, 1H, HCl salt); 12.49 (bs, 1H, NH). M/Z (M+H)⁺: 276.1.

### Example 136: 2-((3-(pyrrolidin-1-yl)propyl)thio)-1,4-dihydroquinazoline dihydrochloride

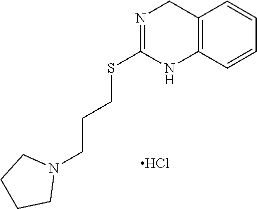 [SMILES: c1ccc2c(c1)CN=C(SCCCN1CCCC1)N2 ; Cl]

Crude example 136 was obtained by concentration to dryness of the reaction mixture. The residue was passed through an ISOLUTE® SCX-2 cartridge (MeOH, then NH₃2 N in MeOH). To a solution of the resulting residue in DCM (2.0 mL) was added HCl in Et₂O (2.0 equiv) and, after evaporation to dryness, the resulting crude yellow solid was purified by flash chromatography (KPNH, CyHex 100% to CyHex/EtOAc 0:100) and by preparative HPLC (column B, H₂O+0.1% HCOOH/MeCN+0.1% HCOOH 95:5 to 55:45) and freeze-dried with 1 N aqueous HCl (5.0 equiv) to afford a white solid (40 mg, 17%).

¹H-NMR (DMSO-d₆, 300 MHz) δ: 1.83-2.02 (m, 4H, 2 CH₂); 2.08-2.19 (m, 2H, N—CH₂—CH₂); 2.87-3.17 (m, 2H, N—CH₂); 3.23 (t, J7.6 Hz, 2H, N—CH₂); 3.40-3.65 (m, 4H, N—CH₂+S-CH₂); 4.73 (s, 2H, N—CH₂—Ar); 7.19-7.26 (m, 3H, 3 Ar); 7.29-7.36 (m, 1H, Ar); 10.86 (bs, 2H, 2 HCl salts); 12.50 (bs, 1H, NH). M/Z (M+H)⁺: 276.1. Mp: 178-182° C.

### Example 137: 2-((4-(pyrrolidin-1-yl)butyl)thio)-1,4-dihydroquinazoline dihydrochloride

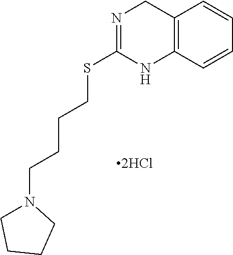 [SMILES: c1ccc2c(c1)CN=C(SCCCCN1CCCC1)N2 ; "·2HCl"]

Crude example 137 was obtained by concentration to dryness of the reaction mixture. The residue was passed through an ISOLUTE® SCX-2 cartridge (MeOH/DCM, then NH₃2 N in MeOH). To a solution of the resulting residue in DCM (2.0 mL) was added HCl in Et₂O (2.0 equiv) and, after evaporation to dryness, the resulting crude brown solid was purified by flash chromatography (KPNH, CyHex 100% to CyHex/EtOAc 0:100), by preparative HPLC (column B, H₂O+0.1% HCOOH/MeCN+0.1% HCOOH 95:5 to 55:45) and freeze-dried with 1 N aqueous HCl (5.0 equiv) to afford a white hygroscopic solid (58 mg, 23%).

¹H-NMR (DMSO-d₆, 300 MHz) δ: 1.70-1.84 (m, 4H, 2 CH₂); 1.86-2.03 (m, 4H, 2 CH₂); 2.84-3.07 (m, 2H, N—CH₂); 3.11-3.17 (m, 2H, N—CH₂); 3.38-3.56 (m, 4H, N—CH₂+S-CH₂); 4.72 (s, 2H, N—CH₂—Ar); 7.19-7.25 (m, 3H, 3 Ar); 7.29-7.36 (m, 1H, Ar); 10.77 (bs, 2H, HCl salts); 12.48 (bs, 1H, NH). M/Z (M+H)⁺: 290.2. Mp: 78-85° C.

### Example 138: 2-((2-(3-methylpyrrolidin-1-yl)ethyl)thio)-1,4-dihydroquinazoline dihydrochloride

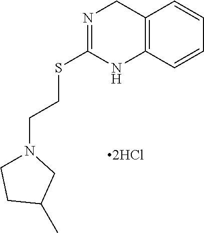 [SMILES: CC1CCN(CCSC2=NCc3ccccc3N2)C1]

Crude example 138 was obtained by concentration to dryness of the reaction mixture. The residue was passed through an ISOLUTE® SCX-2 cartridge (MeOH/DCM, then NH₃2 N in MeOH). To a solution of the resulting residue in DCM (2.0 mL) was added HCl in Et₂O (2.0 equiv) and, after evaporation to dryness, the resulting crude brown oil was purified by flash chromatography (KPNH, CyHex 100% to CyHex/EtOAc 0:100) and freeze-dried with 1 N aqueous HCl (5.0 equiv). The residue was further purified by preparative HPLC (column B, H₂O+0.1% HCOOH/MeCN+0.1% HCOOH 95:5 to 55:45) and freeze-dried with 1 N aqueous HCl (5.0 equiv). The residue was then further purified by Sephadex LH20 (MeOH 100%) to obtain a white hygroscopic solid (21 mg, 5% over 2 steps). ¹H-NMR (DMSO-d₆, 300 MHz) δ: 1.83-2.02 (m, 4H, 2 CH₂); 2.08-2.19 (m, 2H, N—CH₂—CH₂); 2.87-3.17 (m, 2H, N—CH₂); 3.23 (t, J7.6 Hz, 2H, N—CH₂); 3.40-3.65 (m, 4H, N—CH₂+S-CH₂); 4.73 (s, 2H, N—CH₂—Ar); 7.19-7.26 (m, 3H, 3 Ar); 7.29-7.36 (m, 1H, Ar); 10.86 (bs, 2H, 2 HCl salts); 12.50 (bs, 1H, NH). M/Z (M+H)⁺: 276.1. Mp: 178-182° C.

### Example 139: (1S,4S)-5-(2-((1,4-dihydroquinazolin-2-yl)thio)ethyl)-2-oxa-5-azabicyclo[2.2.1]heptane dihydrochloride

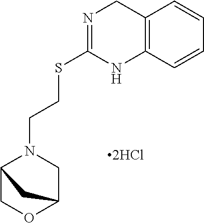 [SMILES: c1ccc2c(c1)CN=C(SCCN1C[C@H]3C[C@@H]1CO3)N2]

Crude example 139 was obtained by concentration to dryness of the reaction mixture. The residue was passed through an ISOLUTE® SCX-2 cartridge (MeOH, then NH₃2 N in MeOH). To a solution of the resulting residue in DCM (2.0 mL) was added HCl in Et₂O (2.0 equiv) and, after evaporation to dryness, the resulting crude yellow solid was purified by preparative HPLC (column B, H₂O+0.1% HCOOH/MeCN+0.1% HCOOH 95:5 to 55:45) and freeze-dried with 1 N aqueous HCl (5.0 equiv) to obtain an orange solid (113 mg, 61%).

¹H-NMR (DMSO-d₆, 300 MHz) δ: 1.96-2.35 (m, 2H, CH₂); 3.03-3.31 (m, 1H, N—CH); 3.46-3.74 (m, 4H, 2 N—CH₂); 3.79-3.99 (m, 2H, S—CH₂); 4.21-4.28 (m, 1H, O-CH); 4.55-4.71 (m, 2H, O-CH₂); 4.73 (s, 2H, N—CH₂—Ar); 7.23-7.29 (m, 3H, 3 Ar); 7.30-7.37 (m, 1H, Ar); 11.06 (bs, 1H, HCl salt); 11.43 (bs, 0.5H, one rotamer of HCl salt); 11.79 (bs, 0.5H, other rotamer of HCl salt); 12.75 (bs, 1H, NH). M/Z (M+H)⁺: 290.1.

### Example 140: 2-((2-(3-phenylpyrrolidin-1-yl)ethyl)thio)-1,4-dihydroquinazoline dihydrochloride

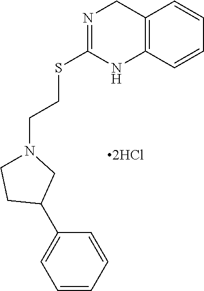 [SMILES: c1ccc(C2CCN(CCSC3=NCc4ccccc4N3)C2)cc1]

Crude example 140 was obtained by concentration to dryness of the reaction mixture. The residue was passed through an ISOLUTE® SCX-2 cartridge (MeOH/DCM, then NH₃2 N in MeOH). To a solution of the resulting crude in DCM (2.0 mL) was added HCl in Et₂O (2.0 equiv) and, after evaporation to dryness, the resulting solid was dissolved in 1 N aqueous HCl (10 mL) and washed with DCM (2×10 mL). The aqueous layer was freeze-dried, then purified by preparative HPLC (column B, H₂O+0.1% HCOOH/MeCN+0.1% HCOOH 95:5 to 55:45) and freeze-dried with 1 N aqueous HCl (5.0 equiv) to obtain a light-yellow solid (140 mg, 30% over 3 steps).

¹H-NMR (DMSO-d₆, 300 MHz) δ: 1.94-2.20 (m, 1.5H, one rotamer of CH-Ar+CH—CHₐH_{b}); 2.37-2.47 (m, 1.5H, other rotamer of CH-Ar+CH—CHₐH_{b}); 3.16-3.31 (m, 1H, N—CHₐH_{b}); 3.57-3.76 (m, 4H, 2 N—CH₂); 3.77-3.99 (m, 3H, S—CH₂+N-CHₐH_{b}); 4.74 (s, 2H, N—CH₂—Ar); 7.20-7.43 (m, 9H, 9 Ar); 10.97 (bs, 1H, HCl salt); 11.28-11.72 (m, 1H, HCl salt); 12.65 (bs, 1H, NH). M/Z (M+H)⁺: 338.2.

### Example 141: 2-(((2R)-2-(pyrrolidin-1-yl)cyclopentyl)thio)-1,4-dihydroquinazoline dihydrochloride

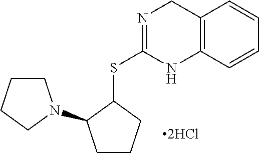 [SMILES: c1ccc2c(c1)CN=C(SC1CCC[C@H]1N1CCCC1)N2]

Crude example 141 was obtained by concentration to dryness of the reaction mixture. The residue was passed through an ISOLUTE® SCX-2 cartridge (MeOH/DCM, then NH₃ 2 N in MeOH). To a solution of the resulting residue in DCM (2.0 mL) was added HCl in Et₂O (2.0 equiv) and, after evaporation to dryness, the resulting yellow solid was purified by preparative HPLC (column B, H₂O+0.1% HCOOH/MeCN+0.1% HCOOH 95:5 to 55:45) and freeze-dried with 1 N aqueous HCl (5.0 equiv) to obtain a white solid (147 mg, 61% over 2 steps).

¹H-NMR (DMSO-d₆, 400 MHz) δ: 1.66-1.83 (m, 2H, CH₂); 1.84-2.10 (m, 6H, 3 CH₂); 2.14-2.24 (m, 1H, CHₐH_{b}); 2.38-2.46 (m, 1H, CHₐH_{b}); 2.97-3.21 (m, 2H, N—CH₂); 3.48-3.59 (m, 1H, N—CH); 3.73-3.92 (m, 2H, N—CH₂); 4.70-4.79 (m, 2H, N—CH₂—Ar); 4.81-4.90 (m, 1H, S—CH); 7.21-7.28 (m, 3H, 3 Ar); 7.30-7.36 (m, 1H, Ar); 11.16 (bs, 1H, HCl salt); 11.35 (bs, 1H, HCl salt); 12.84 (bs, 1H, NH). M/Z (M+H)⁺: 302.1.

### Example 142: 2-((2-(2-azaspiro[4.4]nonan-2-yl)ethyl)thio)-1,4-dihydroquinazoline dihydrochloride

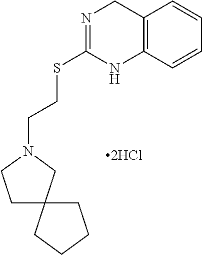 [SMILES: c1ccc2c(c1)CN=C(SCCN1CCC3(CCCC3)C1)N2]

Crude example 142 was obtained by concentration to dryness of the reaction mixture. The residue was passed through an ISOLUTE® SCX-2 cartridge (MeOH/DCM, then NH₃ 2 N in MeOH). To a solution of the resulting residue in DCM (2.0 mL) was added HCl in Et₂O (2.0 equiv) and, after evaporation to dryness, the resulting yellow solid was purified by preparative HPLC (column B, H₂O+0.1% HCOOH/MeCN+0.1% HCOOH 95:5 to 55:45) and freeze-dried with 1 N aqueous HCl (5.0 equiv) to obtain a yellow hygroscopic solid (178 mg, 55%).

¹H-NMR (DMSO-d₆, 300 MHz) δ: 1.53-1.61 (m, 8H, 4 CH₂); 1.80-2.03 (m, 2H, CH₂); 2.99-3.30 (m, 2H, N—CH₂); 3.39-3.49 (m, 4H, 2 N—CH₂); 3.77-3.85 (m, 2H, S—CH₂); 4.74 (s, 2H, N—CH₂—Ar); 7.21-7.28 (m, 3H, 3 Ar); 7.30-7.37 (m, 1H, Ar); 10.94 (bs, 1H, HCl salt); 11.17 (bs, 1H, HCl salt); 12.63 (bs, 1H, NH). M/Z (M+H)⁺: 316.2.

### Example 143: 2-((2-(3-(benzyloxy)pyrrolidin-1-yl)ethyl)thio)-1,4-dihydroquinazoline dihydrochloride

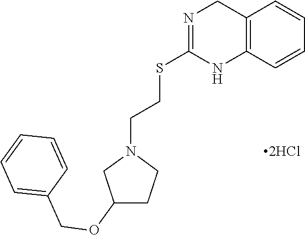 [SMILES: c1ccc(COC2CCN(CCSC3=NCc4ccccc4N3)C2)cc1]

Crude example 143 was obtained by concentration to dryness of the reaction mixture. The residue was passed through an ISOLUTE® SCX-2 cartridge (MeOH/DCM, then NH₃ 2 N in MeOH). To a solution of the resulting residue in DCM (2.0 mL) was added HCl in Et₂O (2.0 equiv) and, after evaporation to dryness, the resulting brown solid was purified by preparative HPLC (column B, H₂O+0.1% HCOOH/MeCN+0.1% HCOOH 95:5 to 55:45) and freeze-dried with 1 N aqueous HCl (2.0 equiv) to obtain a white solid (186 mg, 52%).

¹H-NMR (DMSO-d₆+D₂O, 300 MHz) δ: 2.06-2.25 (m, 2H, CH₂); 3.20-3.37 (m, 2H, N—CH₂); 3.41-3.48 (m, 4H, 2 N—CH₂); 3.50-3.62 (m, 2H, O—CH₂); 4.29-4.35 (m, 1H, O—CH); 4.44-4.49 (m, 2H, S—CH₂); 4.54-4.58 (m, 2H, N—CH₂—Ar); 6.94-7.06 (m, 2H, 2 Ar); 7.11-7.25 (m, 2H, 2 Ar); 7.26-7.34 (m, 5H, 5 Ar). M/Z (M+H)⁺: 368.1.

### Example 144: 1-(2-((1,4-dihydroquinazolin-2-yl)thio)ethyl)pyrrolidine-3-carboxylic acid dihydrochloride

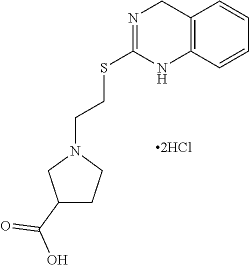 [SMILES: O=C(O)C1CCN(CCSC2=NCc3ccccc3N2)C1]

Crude example 144 was obtained by concentration to dryness of the reaction mixture. The residue was passed through an ISOLUTE® SCX-2 cartridge (MeOH/DCM, then NH₃2 N in MeOH). To a solution of the resulting residue in DCM (2.0 mL) was added HCl in Et₂O (2.0 equiv) and, after evaporation to dryness, the resulting solid was dissolved in 1 N aqueous HCl (10 mL) and washed with DCM (2×10 mL). The aqueous layer was freeze-dried, then purified by preparative HPLC (column B, H₂O+0.1% HCOOH/MeCN+0.1% HCOOH 95:5 to 55:45) and freeze-dried with 1 N aqueous HCl (2.0 equiv) to obtain a colorless hygroscopic solid (119 mg, 17% over 4 steps).

¹H-NMR (DMSO-d₆, 300 MHz) δ: 1.91-2.38 (m, 2H, CH₂); 3.09-3.44 (m, 3H, CH+N-CH₂); 3.52-3.89 (m, 6H, 2 N-CH₂+S-CH₂); 4.73 (s, 2H, N—CH₂—Ar); 7.08-7.18 (m, 0.5H, one rotamer of COOH); 7.21-7.26 (m, 3H, 3 Ar); 7.30-7.36 (m, 1H, Ar); 7.42-7.71 (m, 0.5H, other rotamer of COOH); 10.94-11.19 (m, 1H, HCl salt); 11.45 (bs, 1H, HCl salt); 12.63-12.83 (m, 1H, NH). M/Z (M+H)⁺: 306.1.

### Example 145: 2-((2-(1-methylpyrrolidin-3-yl)ethyl)thio)-1,4-dihydroquinazoline dihydrochloride

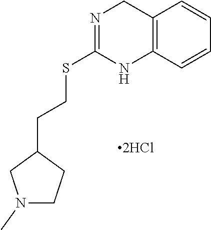 [SMILES: CN1CCC(CCSC2=NCc3ccccc3N2)C1]

Crude example 145 was obtained by concentration to dryness of the reaction mixture. The residue was passed through an ISOLUTE® SCX-2 cartridge (MeOH, then NH₃2 N in MeOH). To a solution of the resulting residue in DCM (2.0 mL) was added HCl in Et₂O (2.0 equiv) and, after evaporation to dryness, the resulting solid was purified by preparative HPLC (column B, H₂O+0.1% HCOOH/MeCN+0.1% HCOOH 90:10) and freeze-dried with 1 N aqueous HCl (2.0 equiv) to obtain a yellow solid (188 mg, 36% over 2 steps).

¹H-NMR (DMSO-d₆, 400 MHz) δ: 1.59-1.69 (m, 0.5H, one rotamer of CH—CH₂); 1.71-1.95 (m, 2.5H, CH₂+other rotamer of CH—CH₂); 2.07-2.16 (m, 0.5H, one rotamer of CHₐH_{b}); 2.18-2.26 (m, 0.5H, other rotamer of CHₐH_{b}); 2.36-2.45 (m, 0.5H, one rotamer of CHₐH_{b}); 2.54-2.58 (m, 0.5H, other rotamer of CHₐH_{b}); 2.66-2.72 (m, 0.5H, one rotamer of N—CHₐH_{b}); 2.75-2.77 (m, 3H, N—CH₃); 2.92-3.18 (m, 2H, N—CH₂); 3.25-3.31 (m, 0.5H, other rotamer of N—CHₐH_{b}); 3.40-3.53 (m, 3H, S—CH₂+N-CHₐH_{b}); 4.71 (s, 1H, one rotamer of N—CH₂—Ar); 4.73 (s, 1H, other rotamer of N—CH₂—Ar); 7.22-7.25 (m, 3H, 3 Ar); 7.28-7.35 (m, 1H, Ar); 10.78-10.85 (m, 1H, HCl salt); 10.97-11.13 (m, 1H, HCl salt); 12.52-12.59 (m, 1H, NH). M/Z (M+H)⁺: 276.1.

### Example 146: (1R,4R)-5-(2-((1,4-dihydroquinazolin-2-yl)thio)ethyl)-2-oxa-5-azabicyclo[2.2.1]heptane dihydrochloride

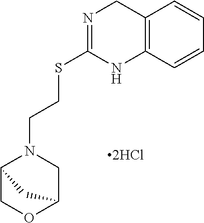 [SMILES: c1ccc2c(c1)CN=C(SCCN1C[C@@H]3C[C@H]1CO3)N2]

Crude example 146 was obtained by concentration to dryness of the reaction mixture. The residue was passed through an ISOLUTE® SCX-2 cartridge (MeOH, then NH₃2 N in MeOH). To a solution of the resulting residue in DCM (2.0 mL) was added HCl in Et₂O (2.0 equiv) and, after evaporation to dryness, the resulting solid was purified by preparative HPLC (column B, H₂O+0.1% HCOOH/MeCN+0.1% HCOOH 90:10) and freeze-dried with 1 N aqueous HCl (2.0 equiv) to obtain a yellow hygroscopic solid (147 mg, 57%).

¹H-NMR (DMSO-d₆, 400 MHz) δ:1.95-2.18 (m, 1.5H, CHₐH_{b}+one rotamer of CHₐH_{b}); 2.27-2.34 (m, 0.5H, other rotamer of CHₐH_{b}); 3.04-3.14 (m, 0.5H, one rotamer of N—CHₐH_{b}); 3.23-3.30 (m, 0.5H, other rotamer of N—CHₐH_{b}); 3.45-3.59 (m, 1.5H, N—CHₐH_{b}+one rotamer of N-CH_{c}H_{d}); 3.61-3.69 (m, 1.5H, N-CH_{c}H_{d}+one rotamer of N-CH_{c}H_{d}); 3.70-3.74 (m, 1H, N—CH); 3.80-3.89 (m, 1H, S—CHₐH_{b}); 3.91-3.95 (m, 1H, S—CHₐH_{b}); 4.17-4.28 (m, 1H, O—CH); 4.55- 4.70 (m, 2H, O—CH₂); 4.73 (s, 2H, N—CH₂—Ar); 7.22-7.29 (m, 3H, 3 Ar); 7.30-7.36 (m, 1H, Ar); 11.04 (bs, 1H, HCl salt); 11.44 (bs, 0.5H, one rotamer of HCl salt); 11.78 (bs, 0.5H, other rotamer of HCl salt); 12.74 (bs, 1H, NH). M/Z (M+H)⁺: 290.1.

### Example 147: 4-((1,4-dihydroquinazolin-2-yl)thio)-1-(pyrrolidin-1-yl)butan-1-one hydrochloride

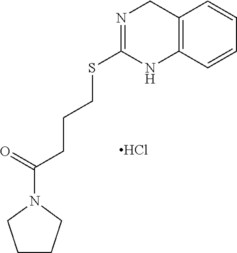 [SMILES: O=C(CCCSC1=NCc2ccccc2N1)N1CCCC1 ; Cl]

Crude example 147 was obtained by concentration to dryness of the reaction mixture. The residue was passed through an ISOLUTE® SCX-2 cartridge (MeOH, then NH₃2 N in MeOH). To a solution of the resulting residue in DCM (2.0 mL) was added HCl in Et₂O (2.0 equiv) and, after evaporation to dryness, the resulting solid was purified by preparative HPLC (column A, H₂O+0.1% HCOOH/MeCN+0.1% HCOOH 95:5 to 55:45) and freeze-dried with 1 N aqueous HCl (2.0 equiv). The residue was dissolved in water, then washed with DCM (2×10 mL), then the aqueous layer was freeze-dried to obtain a yellow hygroscopic solid (121 mg, 63%).

¹H-NMR (DMSO-d₆, 300 MHz) δ: 1.72-1.81 (m, 2H, CH₂); 1.83-1.97 (m, 4H, 2 CH₂); 2.44 (t, J6.2 Hz, 2H, CO—CH₂); 3.31 (t, J7.1 Hz, 4H, 2 N—CH₂); 3.38 (t, J 6.4 Hz, 2H, S—CH₂); 4.72 (s, 2H, N—CH₂—Ar); 7.19-7.26 (m, 3H, 3 Ar); 7.30-7.38 (m, 1H, Ar); 10.70 (bs, 1H, HCl salt); 12.36 (bs, 1H, NH). M/Z (M+H)⁺: 304.1.

### Example 148: 2-(((2R)-2-(pyrrolidin-1-yl)cyclohexyl)thio)-1,4-dihydroquinazoline dihydrochloride

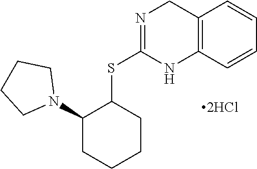 [SMILES: c1ccc2c(c1)CN=C(SC1CCCC[C@H]1N1CCCC1)N2]

Crude example 148 was obtained by concentration to dryness of the reaction mixture. The residue was passed through an ISOLUTE® SCX-2 cartridge (MeOH, then NH₃2 N in MeOH). To a solution of the resulting residue in DCM (2.0 mL) was added HCl in Et₂O (2.0 equiv) and, after evaporation to dryness, the resulting yellow oil was purified thrice by preparative HPLC (column B, H₂O+0.1% HCOOH/MeCN+0.1% HCOOH 95:5 to 55:45, then column B, H₂O+0.1% HCOOH/MeCN+0.1% HCOOH 85:15, then column C, H₂O+0.1% HCOOH/MeCN+0.1% HCOOH 95:5 to 0:100) and freeze-dried with 1 N aqueous HCl (2.0 equiv) to obtain a white hygroscopic solid (52 mg, 23% over 2 steps).

¹H-NMR (DMSO-d₆, 300 MHz) δ: 1.43-1.60 (m, 3H, CH₂+one isomer of CHₐH_{b}); 1.70-2.08 (m, 9H, 4 CH₂+other isomer of CHₐH_{b}); 3.10-3.27 (m, 2H, N—CH₂); 3.47-3.64 (m, 3H, N—CH₂+N-CH); 4.68-4.79 (m, 3H, S-CH+N-CH₂—Ar); 7.22-7.30 (m, 3H, 3 Ar); 7.31-7.37 (m, 1H, Ar); 10.52 (bs, 1H, HCl salt); 11.31 (bs, 1H, HCl salt); 12.93 (bs, 1H, NH). M/Z (M+H)⁺: 316.1.

### Example 149: 5-fluoro-2-((4-(pyrrolidin-1-yl)butyl)thio)-1,4-dihydroquinazoline dihydrochloride

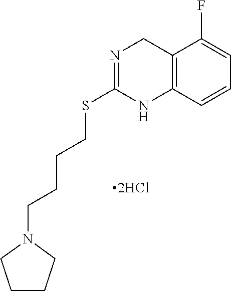 [SMILES: Fc1cccc2c1CN=C(SCCCCN1CCCC1)N2 ; "·2HCl"]

Crude example 149 was obtained by concentration to dryness of the reaction mixture. The residue was passed through an ISOLUTE® SCX-2 cartridge (DCM/MeOH, then NH₃ 2 N in MeOH). To a solution of the crude in DCM (2.0 mL) was added HCl in Et₂O (2.0 equiv) and, after evaporation to dryness, the residue was purified by flash chromatography (KPNH, CyHex 100% to CyHex/EtOAc 0:100). The residue was dissolved in water and 1 N aqueous HCl, then washed with DCM (2×10 mL), then the aqueous layer was freeze-dried to obtain a white solid (71 mg, 54%).

¹H-NMR (DMSO-d₆, 300 MHz) δ: 1.70-1.83 (m, 4H, 2 CH₂); 1.86-2.01 (m, 4H, 2 CH₂); 2.85-3.04 (m, 2H, N—CH₂); 3.07-3.18 (m, 3H, N—CH₂+N-CHₐH_{b}); 3.25-3.32 (m, 3H, N—CHₐH_{b}+S-CH₂); 4.73 (s, 2H, N—CH₂—Ar); 7.00-7.03 (m, 1H, Ar); 7.07-7.12 (m, 1H, Ar); 7.33-7.41 (m, 1H, Ar); 10.28-11.01 (m, 2H, 2 HCl salts); 12.57 (bs, 1H, NH). M/Z (M+H)⁺: 308.1.

### Example 150: 7-chloro-2-((4-(pyrrolidin-1-yl)butyl)thio)-1,4-dihydroquinazoline dihydrochloride

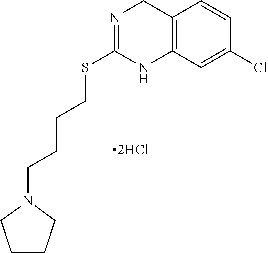 [SMILES: Clc1ccc2c(c1)NC(SCCCCN1CCCC1)=NC2 ; "·2HCl"]

Crude example 150 was obtained by concentration to dryness of the reaction mixture. The residue was passed through an ISOLUTE® SCX-2 cartridge (DCM/MeOH, then NH₃ 2 N in MeOH). To a solution of the crude in DCM (2.0 mL) was added HCl in Et₂O (2.0 equiv) and, after evaporation to dryness, the residue was purified by flash chromatography (KPNH, CyHex 100% to CyHex/EtOAc 1:1). The residue was dissolved in water and 1 N aqueous HCl, then washed with DCM (2×10 mL), then the aqueous layer was freeze-dried to obtain a white solid (55 mg, 40%).

¹H-NMR (DMSO-d₆+D₂O, 300 MHz) δ: 1.63-1.78 (m, 4H, 2 CH₂); 1.83-2.04 (m, 4H, 2 CH₂); 2.84-3.03 (m, 2H, N—CH₂); 3.08-3.16 (m, 2H, N—CH₂); 3.25-3.37 (m, 2H, N—CH₂); 3.40-3.56 (m, 2H, S—CH₂); 4.67 (s, 2H, N—CH₂—Ar); 7.10- 7.18 (m, 1H, Ar); 7.22-7.32 (m, 2H, 2 Ar). M/Z (M[³⁵Cl]+H)⁺: 324.1.

### Example 151: 7-fluoro-2-((4-(pyrrolidin-1-yl)butyl)thio)-1,4-dihydroquinazoline dihydrochloride

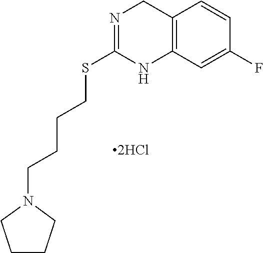 [SMILES: Fc1ccc2c(c1)NC(SCCCCN1CCCC1)=NC2 ; "·2HCl"]

Crude example 151 was obtained by concentration to dryness of the reaction mixture. The residue was passed through an ISOLUTE® SCX-2 cartridge (DCM/MeOH, then NH₃ 2 N in MeOH). To a solution of the crude in DCM (2.0 mL) was added HCl in Et₂O (2.0 equiv) and, after evaporation to dryness, the residue was purified by flash chromatography (KPNH, CyHex 100% to CyHex/EtOAc 1:1). The residue was dissolved in water and 1 N aqueous HCl, then washed with DCM (2×10 mL), then the aqueous layer was freeze-dried to obtain a yellow hygroscopic solid (14 mg, 11%).

¹H-NMR (DMSO-d₆, 300 MHz) δ: 1.72-1.81 (m, 4H, 2 CH₂); 1.84-2.10 (m, 4H, 2 CH₂); 2.85-3.03 (m, 2H, N—CH₂); 3.09-3.19 (m, 3H, N—CH₂+N-CHₐH_{b}); 3.25-3.31 (m, 3H, N—CHₐH_{b}+S-CH₂); 4.69 (s, 2H, N—CH₂—Ar); 7.00-7.04 (m, 1H, Ar); 7.07-7.14 (m, 1H, Ar); 7.28-7.32 (m, 1H, Ar); 10.42 (bs, 1H, HCl salt); 10.78 (bs, 1H, HCl salt); 12.56 (bs, 1H, NH). M/Z (M+H)⁺: 308.1.

### Example 152: 6-fluoro-2-((4-(pyrrolidin-1-yl)butyl)thio)-1,4-dihydroquinazoline dihydrochloride

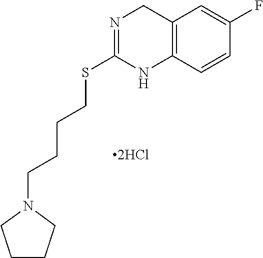 [SMILES: Fc1ccc2c(c1)CN=C(SCCCCN1CCCC1)N2 ; "·2HCl"]

Crude example 152 was obtained by concentration to dryness of the reaction mixture. The residue was passed through an ISOLUTE® SCX-2 cartridge (DCM/MeOH, then NH₃2 N in MeOH). To a solution of the resulting crude in DCM was added HCl in Et₂O (2.0 equiv) and, after evaporation to dryness, the residue was purified by flash chromatography (KPNH, CyHex 100% to CyHex/EtOAc 5:95). The residue was dissolved in water and 1 N aqueous HCl, then washed with DCM (2×10 mL), then the aqueous layer was freeze-dried and purified by preparative HPLC (column C, H₂O+0.1% HCOOH/MeCN+0.1% HCOOH 95:5 to 100:0) and freeze-dried with 1 N aqueous HCl (2.0 equiv) to obtain a white solid (35 mg, 29%).

¹H-NMR (DMSO-d₆, 300 MHz) δ: 1.71-1.83 (m, 4H, 2 CH₂); 1.85-2.06 (m, 4H, 2 CH₂); 2.83-3.04 (m, 2H, N—CH₂); 3.10-3.18 (m, 2H, N—CH₂); 3.40-3.59 (m, 4H, N—CH₂+S-CH₂); 4.72 (s, 2H, N—CH₂—Ar); 7.14-7.29 (m, 3H, 3 Ar); 10.65 (bs, 2H, HCl salts); 12.58 (bs, 1H, NH). M/Z (M+H)⁺: 308.1.

### Example 153: 8-chloro-2-((4-(pyrrolidin-1-yl)butyl)thio)-1,4-dihydroquinazoline dihydrochloride

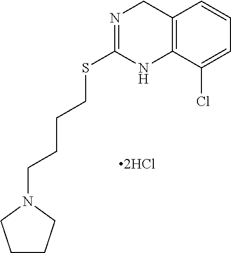 [SMILES: Clc1cccc2c1NC(SCCCCN1CCCC1)=NC2]

Crude example 153 was obtained by concentration to dryness of the reaction mixture. The residue was passed through an ISOLUTE® SCX-2 cartridge (DCM/MeOH, then NH₃2 N in MeOH). To a solution of the resulting crude in DCM was added HCl in Et₂O (2.0 equiv) and, after evaporation to dryness, the residue was purified by flash chromatography (KPNH, CyHex/EtOAc 80:20 to CyHex/EtOAc 0:100). The residue was dissolved in water and 1 N aqueous HCl, then washed with DCM (2×10 mL), then the aqueous layer was freeze-dried to obtain a white solid (49 mg, 35%).

¹H-NMR (DMSO-d₆, 300 MHz) δ: 1.70-1.83 (m, 4H, 2 CH₂); 1.85-2.02 (m, 4H, 2 CH₂); 2.86-3.00 (m, 2H, N—CH₂); 3.08-3.17 (m, 2H, N—CH₂); 3.32-3.40 (m, 2H, N—CH₂); 3.43-3.48 (m, 2H, S—CH₂); 4.67 (s, 2H, N—CH₂—Ar); 7.14-7.26 (m, 2H, 2 Ar); 7.37-7.47 (m, 1H, Ar); 10.78 (bs, 1H, HCl salt); 2^{nd} HCl salt signal and NH signal not observed. M/Z (M[³⁵Cl]+H)⁺: 324.1.

### Example 154: 2-((2-(3-benzylpyrrolidin-1-yl)ethyl)thio)-1,4-dihydroquinazoline dihydrochloride

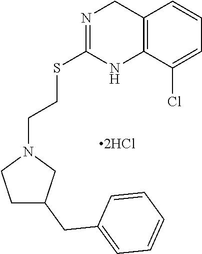 [SMILES: Clc1cccc2c1NC(SCCN1CCC(Cc3ccccc3)C1)=NC2]

Crude example 154 was obtained by concentration to dryness of the reaction mixture. The residue was passed through an ISOLUTE® SCX-2 cartridge (DCM/MeOH, then NH₃2 N in MeOH). To a solution of the resulting crude in DCM (2 mL) was added HCl in Et₂O (2.0 equiv) and, after evaporation to dryness, the resulting brown oil was purified by preparative HPLC (column A, H₂O+0.1% HCOOH/MeCN+0.1% HCOOH 95:5 to 55:45) and freeze-dried with 1 N aqueous HCl (2.0 equiv). The residue was dissolved in water and washed with DCM (2×10 mL), then the aqueous layer was freeze-dried and further purified by Sephadex LH20 (MeOH 100%) and then by preparative HPLC (column A, H₂O+0.1% HCOOH/MeCN+0.1% HCOOH 95:5 to 55:45) and freeze-dried with 1 N aqueous HCl to obtain a white solid (63 mg, 12% over 3 steps).

¹H-NMR (DMSO-d₆, 300 MHz) δ: 1.59-1.78 (m, 1H, CHₐH_{b}); 1.94-2.14 (m, 1H, CHₐH_{b}); 2.67-2.87 (m, 3H, Ar—CH₂—CH+Ar—CH₂); 3.05-3.32 (m, 2H, N—CH₂); 3.49-3.68 (m, 4H, 2 N—CH₂); 3.72-3.83 (m, 2H, S—CH₂); 4.72 (s, 2H, N—CH₂—Ar); 7.17-7.27 (m, 6H, 6 Ar); 7.28-7.39 (m, 3H, 3 Ar); 10.76-11.40 (m, 2H, HCl salts); 12.60 (bs, 1H, NH). M/Z (M+H)⁺: 352.2.

### Example 155: 4-(2-((1,4-dihydroquinazolin-2-yl)thio)ethyl)morpholine dihydrochloride

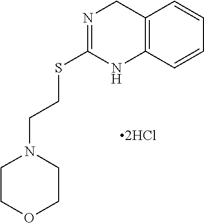 [SMILES: c1ccc2c(c1)CN=C(SCCN1CCOCC1)N2]

Crude example 155 was obtained by concentration to dryness of the reaction mixture. The residue was passed through an ISOLUTE® SCX-2 cartridge (MeOH, then NH₃3 N in MeOH). To a solution of the resulting crude in DCM (2 mL) was added HCl in Et₂O (2.0 equiv) and, after evaporation to dryness, the resulting beige solid was purified by preparative HPLC (column B, H₂O+0.1% HCOOH/MeCN+0.1% HCOOH 100:0 to 0:100) and freeze-dried with 1 N aqueous HCl (5.0 equiv) to obtain a light-yellow solid (165 mg, 59%).

¹H-NMR (DMSO-d₆, 400 MHz) δ: 3.07-3.23 (m, 2H, N—CH₂); 3.44-3.55 (m, 4H, 2 N—CH₂); 3.78-4.04 (m, 6H, 2 N-CH₂+S-CH₂); 4.74 (s, 2H, N—CH₂—Ar); 7.24-7.28 (m, 3H, 3 Ar); 7.31-7.36 (m, 1H, Ar); 10.99 (bs, 1H, HCl salt); 11.49 (bs, 1H, HCl salt); 12.68 (bs, 1H, NH). M/Z (M+H)⁺: 278.1.

### Example 156: (S)-2-((2-(3-fluoropyrrolidin-1-yl)ethyl)thio)-1,4-dihydroquinazoline dihydrochloride

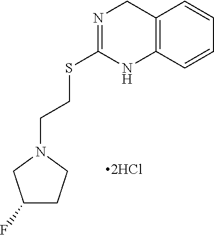 [SMILES: F[C@H]1CCN(CCSC2=NCc3ccccc3N2)C1]

Crude example 156 was obtained by concentration to dryness of the reaction mixture. The residue was passed through an ISOLUTE® SCX-2 cartridge (MeOH, then NH₃3 N in MeOH). To a solution of the resulting crude in DCM (2 mL) was added HCl in Et₂O (2.0 equiv) and, after evaporation to dryness, the resulting brown solid was purified by preparative HPLC (column C, H₂O+0.1% HCOOH/MeCN+0.1% HCOOH 95:5 to 0:100) and freeze-dried with 1 N aqueous HCl (5.0 equiv). The residue was dissolved in water and an aqueous saturated solution of NaHCO₃ and was extracted with DCM (2×20 mL). 1 N HCl in Et₂O (2.0 equiv) was added to the combined organic layers that were concentrated to dryness and then purified by preparative HPLC (column B, H₂O+0.1% HCOOH/MeCN+0.1% HCOOH 95:5 to 55:45) and freeze-dried with 1 N aqueous HCl (2.0 equiv) to obtain a yellow solid (101 mg, 16% over 2 steps).

¹H-NMR (DMSO-d₆, 300 MHz) δ: 2.06-2.34 (m, 1H, CHₐH_{b}); 2.56-2.60 (m, 1H, CHₐH_{b}); 3.25-3.41 (m, 1H, one rotamer of N—CH₂); 3.46-3.66 (m, 3H, N—CH₂+other rotamer of N—CH₂); 3.68-3.75 (m, 1H, one rotamer of N—CH₂); 3.78-3.87 (m, 3H, S—CH₂+other rotamer of N—CH₂); 4.73 (s, 2H, N—CH₂—Ar); 5.39 (bs, 0.5H, one rotamer of F—CH); 5.57 (bs, 0.5H, other rotamer of F—CH); 7.20-7.29 (m, 3H, 3 Ar); 7.30-7.37 (m, 1H, Ar); 11.00 (bs, 1H, HCl salt); 11.32-11.64 (m, 1H, HCl salt); 12.67 (bs, 1H, NH). M/Z (M+H)⁺: 280.2.

### Example 157: (R)-2-((2-(3-fluoropyrrolidin-1-yl)ethyl)thio)-1,4-dihydroquinazoline dihydrochloride

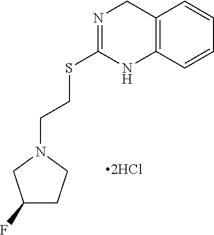 [SMILES: F[C@@H]1CCN(CCSC2=NCc3ccccc3N2)C1]

Crude example 157 was obtained by concentration to dryness of the reaction mixture. The residue was passed through an ISOLUTE® SCX-2 cartridge (MeOH, then NH₃3 N in MeOH). To a solution of the crude in DCM (2 mL) was added HCl in Et₂O (2.0 equiv) and, after evaporation to dryness, the resulting brown solid was purified by preparative HPLC (column C, H₂O+0.1% HCOOH/MeCN+0.1% HCOOH 95:5 to 0:100) and freeze-dried with 1 N aqueous HCl (5.0 equiv). The residue was dissolved in water and washed with DCM (2×10 mL), then the aqueous layer was freeze-dried, further purified by preparative HPLC (column B, H₂O+0.1% HCOOH/MeCN+0.1% HCOOH 95:5 to 55:45) and freeze-dried with 1 N aqueous HCl (5.0 equiv) to obtain a yellow solid (84 mg, 15% over 2 steps).

¹H-NMR (DMSO-d₆, 300 MHz) δ: 2.09-2.25 (m, 0.5H, one rotamer of CHₐH_{b}); 2.55-2.60 (m, 1.5H, other rotamer of CHₐH_{b}); 3.25-3.40 (m, 1H, one rotamer of N—CH₂); 3.48-3.63 (m, 3H, N—CH₂+other rotamer of N—CH₂); 3.78-3.94 (m, 4H, N—CH₂+S-CH₂); 4.73 (s, 2H, N—CH₂—Ar); 5.39 (bs, 0.5H, one rotamer of F—CH); 5.57 (bs, 1H, other rotamer of F—CH); 7.21-7.28 (m, 3H, 3 Ar); 7.30-7.38 (m, 1H, Ar); 10.97 (bs, 1H, HCl salt); 11.30-11.58 (m, 1H, HCl salt); 12.66 (bs, 1H, NH). M/Z (M+H)⁺: 279.9.

### Example 158: 6-chloro-2-((2-(1-methylpyrrolidin-2-yl)ethyl)thio)-1,4-dihydroquinazoline dihydrochloride

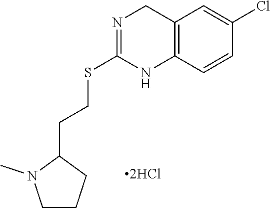 [SMILES: CN1CCCC1CCSC1=NCc2cc(Cl)ccc2N1]

Crude example 158 was obtained by filtration of the reaction mixture. The solid was passed through an ISOLUTE® SCX-2 cartridge (water/MeOH, then NH₃1 N in MeOH). To a solution of the residue in DCM (10 mL) was added HCl in Et₂O (1 mL) and, after evaporation to dryness, the resulting crude was purified by flash chromatography (DCM 100% to DCM/MeOH 80:20). HCl 2 M in Et₂O (1 mL) was added to the resulting residue that was then concentrated to dryness and freeze-dried in water to obtain an off-white solid (153 mg, 79%).

¹H-NMR (D₂O, 300 MHz) δ: 1.73-1.86 (m, 1H, CHₐH_{b}); 1.95-2.21 (m, 3H, CHₐH_{b}+CH₂); 2.30-2.51 (m, 2H, CH₂); 2.91 (s, 3H, N—CH₃); 3.09-3.54 (m, 4H, N—CH₂+S-CH₂); 3.62-3.78 (m, 1H, N—CH); 4.74 (s, 2H, N—CH₂—Ar); 7.01 (d, J8.6 Hz, 1H, Ar); 7.23 (d, J 2.2 Hz, 1H, Ar); 7.35 (dd, J 8.6, 2.2 Hz, 1H, Ar). M/Z (M[³⁵Cl]+H)⁺: 310.2.

### Example 159: 2-((4-(pyrrolidin-1-yl)butyl)thio)-4,5-dihydro-1H-benzo[d][1,3]diazepine dihydrochloride

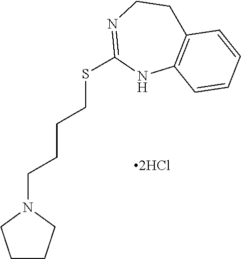 [SMILES: c1ccc2c(c1)CCN=C(SCCCCN1CCCC1)N2]

Crude example 159 was obtained by filtration of the reaction mixture, followed by washing of the solid with DCM (20 mL). The solid was then passed through an ISOLUTE® SCX-2 cartridge (MeOH, then NH₃3.5 N in MeOH). To a solution of the resulting residue in DCM (10 mL) was added HCl in Et₂O (2.0 equiv) and, after evaporation to dryness, the residue was freeze-dried in water to obtain a white solid (65 mg, 51%).

¹H-NMR (CD₃OD, 300 MHz) δ: 1.81-2.00 (m, 4H, 2 CH₂); 2.03-2.17 (m, 4H, 2 CH₂); 2.99-3.18 (m, 2H, Ar—CH₂r); 3.23-3.28 (m, 4H, 2 N—CH₂); 3.36 (t, J7.2 Hz, 2H, N—CH₂); 3.57-3.73 (m, 2H, N—CH₂); 3.81-3.84 (m, 2H, S—CH₂); 7.22-7.38 (m, 4H, 4 Ar). M/Z (M+H)⁺: 304.2.

### Example 160: 4,4-dimethyl-2-((4-(pyrrolidin-1-yl)butyl)thio)-1,4-dihydroquinazoline dihydrochloride

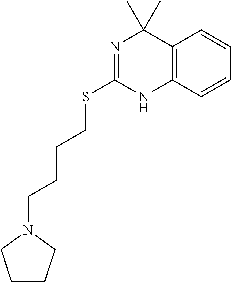 [SMILES: CC1(C)N=C(SCCCCN2CCCC2)Nc2ccccc21]

Crude example 160 was obtained by dilution of the reaction mixture with methanol, followed by elution of the resulting solution through an ISOLUTE® SCX-2 cartridge (MeOH, then NH₃ 3.5 N in MeOH). To a solution of the resulting residue in DCM (10 mL) was added HCl in Et₂O (2.0 equiv) and, after evaporation to dryness, the residue was purified by flash chromatography (DCM 100% to DCM/MeOH 80:20). Then HCl in Et₂O (5 equiv) was added to the residue which was concentrated to dryness and freeze-dried in water to obtain a pale-rose solid (108 mg, 76%).

¹H-NMR (DMSO-d₆, 300 MHz) δ: 1.66 (s, 6H, (CH₃)₂); 1.68-2.02 (m, 8H, 4 CH₂); 2.88-2.99 (m, 2H, N—CH₂); 3.10-3.17 (m, 2H, N—CH₂); 3.43-3.50 (m, 2H, N—CH₂); 3.56 (t, J7.0 Hz, 2H, S—CH₂); 7.26-7.38 (m, 3H, 3 Ar); 7.42-7.75 (m, 1H, Ar); 10.68 (bs, 1H, HCl salt); 10.81 (bs, 1H, HCl salt); 12.71 (bs, 1H, NH). M/Z (M+H)⁺: 318.3.

### Example 161: 6-chloro-2-((3-(pyrrolidin-1-yl)propyl)thio)-1,4-dihydroquinazoline dihydrochloride

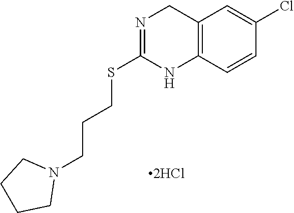 [SMILES: Clc1ccc2c(c1)CN=C(SCCCN1CCCC1)N2]

Crude example 161 was obtained by filtration of the reaction mixture. The solid was passed through an ISOLUTE® SCX-2 cartridge (MeOH, then NH₃7 N in MeOH). To a solution of the residue in DCM (10 mL) was added HCl in Et₂O (2.0 equiv) and, after evaporation to dryness, the resulting crude was dissolved in water (10 mL) and washed with DCM (3×5 mL). Then the aqueous layer was filtrated, freeze-dried, purified by preparative HPLC (column B, H₂O+0.1% HCOOH/MeCN+0.1% HCOOH 90:10) and freeze-dried with 1 N aqueous HCl (2.0 equiv) to obtain a white solid (63 mg, 33%).

¹H-NMR (D₂O, 300 MHz) δ: 1.90-2.25 (m, 6H, 3 CH₂); 2.97-3.17 (m, 2H, N—CH₂); 3.28-3.37 (m, 4H, N—CH₂+S-CH₂); 3.57-3.76 (m, 2H, N—CH₂); 4.73 (s, 2H, N—CH₂—Ar); 6.98-7.01 (m, 1H, Ar); 7.19-7.26 (m, 1H, Ar); 7.33-7.36 (m, 1H, Ar). M/Z (M[³⁵Cl]+H)⁺: 310.2.

### Example 162: 6-chloro-2-((4-(pyrrolidin-1-yl)pentyl)thio)-1,4-dihydroquinazoline dihydrochloride

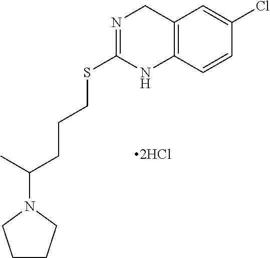 [SMILES: CC(CCCSC1=NCc2cc(Cl)ccc2N1)N1CCCC1]

Crude example 162 was obtained by filtration of the reaction mixture. The solid was passed through an ISOLUTE® SCX-2 cartridge (MeOH, then NH₃7 N in MeOH). To a solution of the residue in DCM (10 mL) was added HCl in Et₂O (2.0 equiv) and, after evaporation to dryness, the resulting crude was dissolved in water (10 mL) and washed with DCM (3×5 mL). Then the aqueous layer was filtrated, freeze-dried, purified by preparative HPLC (column B, H₂O+0.1% HCOOH/MeCN+0.1% HCOOH 100:0 to 90:10) and freeze-dried with 1 N aqueous HCl (2.0 equiv) to obtain an orange solid (38 mg, 18%).

¹H-NMR (D₂O, 300 MHz) δ: 1.32 (d, J6.3 Hz, 3H, CH₃); 1.67-2.02 (m, 6H, 3 CH₂); 2.02-2.16 (m, 2H, CH₂); 3.04-3.18 (m, 2H, N—CH₂); 3.22-3.32 (m, 2H, S—CH₂); 3.32-3.42 (m, 1H, N—CH); 3.50-3.61 (m, 2H, N—CH₂); 4.73 (s, 2H, N—CH₂—Ar); 7.01 (d, J8.6 Hz, 1H, Ar); 7.21-7.26 (m, 1H, Ar); 7.35 (dd, J8.6, 2.3 Hz, 1H, Ar). M/Z (M[³⁵Cl]+H)⁺: 338.2.

### Example 163: 6-bromo-2-((4-(pyrrolidin-1-yl)butyl)thio)-1,4-dihydroquinazoline dihydrochloride

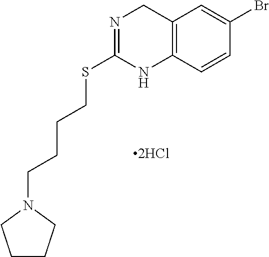 [SMILES: Brc1ccc2c(c1)CN=C(SCCCCN1CCCC1)N2]

Crude example 163 was obtained by filtration of the reaction mixture. The solid was passed through an ISOLUTE® SCX-2 cartridge (MeOH, then NH₃7 N in MeOH). To a solution of the residue in DCM (30 mL) was added HCl in Et₂O (2.0 equiv) and, after evaporation to dryness, the resulting crude was dissolved in water (20 mL) and washed with DCM (3×10 mL). Then the aqueous layer was centrifuged, the supernatant was washed with DCM (20 mL) and the resulting aqueous layer was freeze-dried to obtain a white solid (193 mg, 53%).

¹H-NMR (DMSO-d₆, 300 MHz) δ: 1.71-1.95 (m, 8H, 4 CH₂); 2.88-3.00 (m, 2H, N—CH₂); 3.11-3.15 (m, 2H, N/S—CH₂); 3.47-3.54 (m, 4H, 2 N/S—CH₂); 4.71 (s, 2H, N—CH₂—Ar); 7.17-7.20 (m, 1H, Ar); 7.50-7.54 (m, 2H, 2 Ar); 10.76 (m, 2H, 2 HCl salts); 12.66 (m, 1H, NH). M/Z (M[⁷⁹Br]⁺H)⁺: 368.2.

### Example 164: 6-chloro-2-((4-(piperidin-1-yl)butyl)thio)-1,4-dihydroquinazoline dihydrochloride

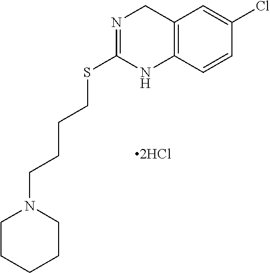 [SMILES: Clc1ccc2c(c1)CN=C(SCCCCN1CCCCC1)N2]

Crude example 164 was obtained by filtration of the reaction mixture. The solid was passed through an ISOLUTE® SCX-2 cartridge (MeOH, then NH₃7 N in MeOH). To a solution of the residue in DCM (10 mL) was added HCl in Et₂O (2.0 equiv) and, after evaporation to dryness, the resulting crude was purified by preparative HPLC (column B, H₂O+0.1% HCOOH/MeCN+0.1% HCOOH 85:15) and freeze-dried with 1 N aqueous HCl (2.0 equiv) to obtain a white foam (66 mg, 35%).

¹H-NMR (D₂O, 300 MHz) δ: 1.37-1.52 (m, 1H, CHₐH_{b}); 1.62-1.95 (m, 9H, 4 CH₂+CHₐH_{b}); 2.85-2.93 (m, 2H, N—CH₂); 3.08-3.12 (m, 2H, N—CH₂); 3.25-3.30 (m, 2H, S—CH₂); 3.47-3.51 (m, 2H, N—CH₂); 3.50-3.61 (m, 2H, N—CH₂); 4.72 (s, 2H, Ar—CH₂); 6.99-7.02 (m, 1H, Ar); 7.20-7.26 (m, 1H, Ar); 7.33-7.37 (m, 1H, Ar). M/Z (M[³⁵Cl]+H)⁺: 338.2.

### Example 165: 2-((4-(pyrrolidin-1-yl)pentyl)thio)-1,4-dihydroquinazoline dihydrochloride

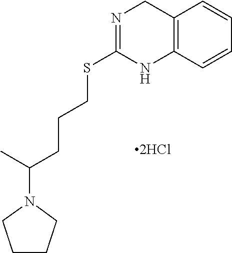 [SMILES: CC(CCCSC1=NCc2ccccc2N1)N1CCCC1]

Crude example 165 was obtained by filtration of the reaction mixture. The solid was passed through an ISOLUTE® SCX-2 cartridge (MeOH, then NH₃7 N in MeOH). To a solution of the residue in DCM (10 mL) was added HCl in Et₂O (2.0 equiv) and, after evaporation to dryness, the resulting crude was purified by preparative HPLC (column B, H₂O+0.1% HCOOH/MeCN+0.1% HCOOH 90:10) and freeze-dried with 1 N aqueous HCl (2.0 equiv) to obtain a white solid (54 mg, 24%).

¹H-NMR (D₂O, 300 MHz) δ: 1.32 (d, J6.6 Hz, 3H, CH₃); 1.68-2.01 (m, 6H, 3 CH₂); 2.01-2.14 (m, 2H, CH₂); 3.04-3.23 (m, 2H, N—CH₂); 3.22-3.32 (m, 2H, S—CH₂); 3.32-3.42 (m, 1H, N—CH); 3.45-3.64 (m, 2H, N—CH₂); 4.75 (s, 2H, Ar—CH₂); 7.05 (dd, J 7.9, 1.2 Hz, 1H, Ar); 7.19-7.21 (m, 1H, Ar); 7.29 (td, J 7.5, 1.2 Hz, 1H, Ar); 7.36 (td, J 7.5, 1.5 Hz, 1H, Ar). M/Z (M+H)⁺: 304.2.

### Example 166: (S)-6-chloro-2-((2-(pyrrolidin-1-yl)propyl)thio)-1,4-dihydroquinazoline dihydrochloride

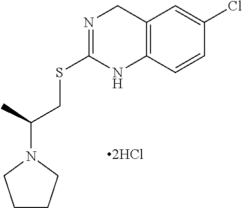 [SMILES: C[C@@H](CSC1=NCc2cc(Cl)ccc2N1)N1CCCC1]

Crude example 166 was obtained by filtration of the reaction mixture. The solid was passed through an ISOLUTE® SCX-2 cartridge (MeOH, then NH₃7 N in MeOH). To a solution of the residue in DCM (10 mL) was added HCl in Et₂O (2.0 equiv) and, after evaporation to dryness, the resulting crude was purified by flash chromatography (DCM 100% to DCM/MeOH 80:20), further purified by preparative HPLC (column B, H₂O+0.1% HCOOH/MeCN+0.1% HCOOH 90:10) and freeze-dried with 1 N aqueous HCl (2.0 equiv) to obtain a white solid (15 mg, 8%).

¹H-NMR (D₂O, 300 MHz) δ: 1.50 (d, J6.6 Hz, 3H, CH₃); 1.91-2.19 (m, 4H, 2 CH₂); 3.06-3.68 (m, 5H, 2 N-CH₂+N-CH); 3.70-3.80 (m, 2H, S—CH₂); 4.76 (s, 2H, N—CH₂—Ar); 7.04 (d, J8.5 Hz, 1H, 1 Ar); 7.25 (d, J2.2 Hz, 1H, 1 Ar); 7.32 (dd, J 8.5, 2.2 Hz, 1H, Ar). M/Z (M[³⁵Cl]+H)⁺: 310.2.

### Example 167: (R)-6-chloro-2-((2-(pyrrolidin-1-yl)propyl)thio)-1,4-dihydroquinazoline dihydrochloride

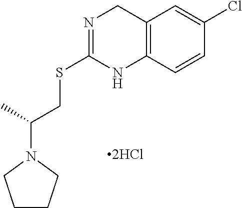 [SMILES: C[C@H](CSC1=NCc2cc(Cl)ccc2N1)N1CCCC1]

Crude example 167 was obtained by filtration of the reaction mixture and washing with MeCN (5 mL). The solid was passed through an ISOLUTE® SCX-2 cartridge (MeOH, then NH₃1 N in MeOH). To a solution of the residue in DCM (10 mL) was added HCl in Et₂O (1 mL) and, after evaporation to dryness, the resulting crude was purified by preparative HPLC (column D, H₂O+0.1% HCOOH/MeCN+0.1% HCOOH 90:10) and freeze-dried with 1 N aqueous HCl (2.0 equiv) to obtain a white solid (54 mg, 43%).

¹H-NMR (D₂O, 300 MHz) δ: 1.51 (d, J6.7 Hz, 3H, CH₃); 1.94-2.24 (m, 4H, 2 CH₂); 3.12-3.31 (m, 2H, N—CH₂); 3.48-3.56 (m, 1H, N—CH); 3.61-3.84 (m, 4H, S—CH₂+N-CH₂); 4.76 (s, 2H, N—CH₂—Ar); 7.04 (d, J8.6 Hz, 1H, Ar); 7.24 (d, J 2.3 Hz, 1H, Ar); 7.37 (dd, J 8.6, 2.3 Hz, 1H, Ar). M/Z (M[³⁵Cl]+H)⁺: 310.1.

### Example 168: (S)-6-chloro-2-((1-(pyrrolidin-1-yl)propan-2-yl)thio)-1,4-dihydroquinazoline dihydrochloride

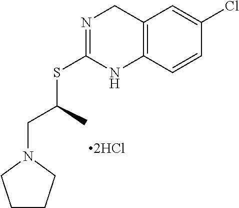 [SMILES: C[C@@H](CN1CCCC1)SC1=NCc2cc(Cl)ccc2N1]

Example 168 was isolated as a by-product during the preparation of example 167 using the same protocol. After the preparative HPLC freeze-drying with 1 N aqueous HCl (5.0 equiv) afforded an off-white solid (7 mg, 6%).

¹H-NMR (D₂O, 300 MHz) δ: 1.61 (d, J6.8 Hz, 3H, CH₃); 1.95-2.33 (m, 4H, 2 CH₂); 3.05-3.93 (m, 6H, 3 N—CH₂); 4.12-4.24 (m, 1H, S—CH); 4.83 (s, 2H, N—CH₂—Ar); 7.07 (d, J8.6 Hz, 1H, Ar); 7.28 (d, J2.3 Hz, 1H, Ar); 7.40 (dd, J8.6, 2.3 Hz, 1H, Ar). M/Z (M[³⁵Cl]+H)⁺: 310.1.

### Example 169: 5-(4-methoxybenzyl)-5-methyl-2-((4-(pyrrolidin-1-yl)butyl)thio)-4,5-dihydro-1H-imidazole dihydrochloride

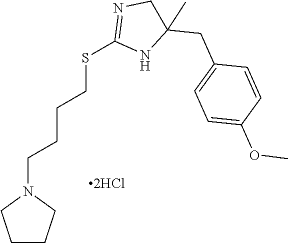 [SMILES: COc1ccc(CC2(C)CN=C(SCCCCN3CCCC3)N2)cc1]

Crude example 169 was obtained by hydrolysis of the reaction mixture with an aqueous saturated solution of NaHCO₃ (15 mL) followed by extraction with EtOAc (3×10 mL). The organic layer was washed with brine (5 mL), dried over magnesium sulfate then concentrated to dryness. The crude was purified by flash chromatography (KPNH, CyHex 100% to CyHex/EtOAc 0:100 then to EtOAc/MeOH 80:20), further purified by preparative HPLC (column B, H₂O+0.1% HCOOH/MeCN+0.1% HCOOH 85:15) and freeze-dried with 1 N aqueous HCl (2.0 equiv) to obtain a colorless oil (36 mg, 26%).

¹H-NMR (DMSO-d₆, 300 MHz) δ: 1.45 (s, 3H, CH₃); 1.50-1.61 (m, 2H, CH₂); 1.69-1.79 (m, 2H, CH₂); 1.83-2.06 (m, 4H, 2 CH₂); 2.67-3.00 (m, 4H, Ar—CH₂+N-CH₂); 3.02-3.25 (m, 4H, 2 N—CH₂); 3.43-3.53 (m, 3H, S—CH₂+N-CHₐH_{b} of imidazoline); 3.74 (s, 3H, O—CH₃); 3.75-3.79 (m, 1H, N—CHₐH_{b} of imidazoline); 6.88-6.91 (m, 2H, 2 Ar); 7.20-7.23 (m, 2H, 2 Ar); 9.98 (s, 1H, HCl salt); 10.70 (s, 1H, HCl salt); 10.76 (bs, 1H, NH). M/Z (M+H)⁺: 362.3.

### Example 170: 5-methyl-5-phenyl-2-((4-(pyrrolidin-1-yl)butyl)thio)-4,5-dihydro-1H-imidazole dihydrochloride

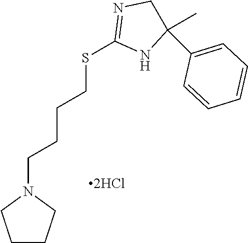 [SMILES: CC1(c2ccccc2)CN=C(SCCCCN2CCCC2)N1]

Crude example 170 was obtained by filtration of the reaction mixture. The solid was passed through an ISOLUTE® SCX-2 cartridge (MeOH, then NH₃3.5 N in MeOH). To a solution of the resulting residue in DCM (6 mL) was added HCl in Et₂O (5.0 equiv) and, after evaporation to dryness, the crude was purified by preparative HPLC (column B, H₂O+0.1% HCOOH/MeCN+0.1% HCOOH 90:10) and freeze-dried with 1 N aqueous HCl (2.0 equiv) to obtain a white solid (55 mg, 44%).

¹H-NMR (DMSO-d₆, 300 MHz) δ: 1.72 (s, 3H, CH₃); 1.75-2.03 (m, 8H, 4 CH₂); 2.87-2.99 (m, 2H, N—CH₂); 3.09-3.15 (m, 2H, N—CH₂); 3.29-3.52 (m, 4H, N—CH₂, S—CH₂); 3.91 (d, J11.1 Hz, 1H, N—CHₐH_{b}); 4.06 (d, J11.1 Hz, 1H, N—CHₐH_{b}); 7.33-7.48 (m, 5H, 5 Ar); 10.70 (bs, 1H, HCl salt); 10.95 (bs, 1H, HCl salt); 11.19 (bs, 1H, NH). M/Z (M+H)⁺: 318.3.

### Example 171: 3-((4-(pyrrolidin-1-yl)butyl)thio)-2,5-dihydro-1H-benzo[e][1,3]diazepine dihydrochloride

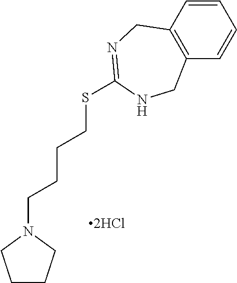 [SMILES: c1ccc2c(c1)CN=C(SCCCCN1CCCC1)NC2]

Crude example 171 was obtained by filtration of the reaction mixture. The solid was passed through an ISOLUTE® SCX-2 cartridge (MeOH, then NH₃3.5 N in MeOH). To a solution of the resulting residue in DCM (10 mL) was added HCl in Et₂O (5.0 equiv) and, after evaporation to dryness, the crude was purified by preparative HPLC (column B, H₂O+0.1% HCOOH/MeCN+0.1% HCOOH 95:05) and freeze-dried with 1 N aqueous HCl (5.0 equiv) to obtain a white solid (90 mg, 53%).

¹H-NMR (DMSO-d₆, 300 MHz) δ: 1.58-1.80 (m, 4H, 2 CH₂); 1.83-2.02 (m, 4H, 2 CH₂); 2.85-2.96 (m, 2H, N—CH₂); 3.04-3.11 (m, 2H, N—CH₂); 3.26 (t, J7.1 Hz, 2H, N/S—CH₂); 3.41-3.52 (m, 2H, N/S—CH₂); 4.79 (d, J4.3 Hz, 4H, 2 N—CH₂); 7.36-7.42 (m, 4H, 4 Ar); 10.31 (bs, 2H, 2 HCl salt); 10.88 (bs, 1H, NH). M/Z (M+H)⁺: 304.2.

### Example 172: 4,4-dimethyl-2-((4-(pyrrolidin-1-yl)butyl)thio)-4,5-dihydro-1H-imidazole dihydrochloride

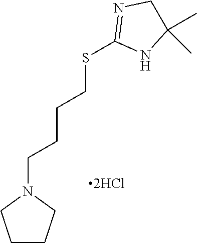 [SMILES: CC1(C)CN=C(SCCCCN2CCCC2)N1]

Crude example 172 was obtained by filtration of the reaction mixture. The solid was passed through an ISOLUTE® SCX-2 cartridge (MeOH, then NH₃3.5 N in MeOH). To a solution of the resulting residue in DCM (10 mL) was added HCl in Et₂O (5.0 equiv) and, after evaporation to dryness, the crude was purified by preparative HPLC (column B, H₂O+0.1% HCOOH/MeCN+0.1% HCOOH 100:0 to 90:10) and freeze-dried with 1 N aqueous HCl (5.0 equiv) to obtain a green oil (112 mg, 56%).

¹H-NMR (DMSO-d₆, 300 MHz) δ: 1.37 (s, 6H, (CH₃)₂); 1.66-2.03 (m, 8H, 4 CH₂); 2.88-2.99 (m, 2H, N—CH₂); 3.08-3.16 (m, 2H, N—CH₂); 3.29 (t, J7.1 Hz, 2H, N/S—CH₂); 3.43-3.52 (m, 2H, N/S—CH₂); 3.61 (s, 2H, N—CH₂); 10.42 (bs, 1H, HCl salt); 10.73 (bs, 1H, HCl salt); 10.97 (bs, 1H, NH). M/Z (M+H)⁺: 256.1.

### Example 173: 2-((4-(pyrrolidin-1-yl)butyl)thio)-1,4,5,6-tetrahydropyrimidine dihydrochloride

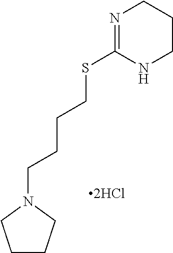 [SMILES: C1CN=C(SCCCCN2CCCC2)NC1]

Crude example 173 was obtained by filtration of the reaction mixture. The solid was passed through an ISOLUTE® SCX-2 cartridge (MeOH, then NH₃3.5 N in MeOH). To a solution of the resulting residue in DCM (10 mL) was added HCl in Et₂O (5.0 equiv) and, after evaporation to dryness, the crude was purified by preparative HPLC (column B, H₂O+0.1% HCOOH/MeCN+0.1% HCOOH 100:0 to 90:10) and freeze-dried with 1 N aqueous HCl (5.0 equiv) to obtain a green gum (69 mg, 32%).

¹H-NMR (DMSO-d₆, 300 MHz) δ: 1.64-2.05 (m, 10H, 5 CH₂); 2.86-3.01 (m, 2H, N—CH₂); 3.08-3.14 (m, 2H, N—CH₂); 3.26 (t, J7.0 Hz, 2H, N/S—CH₂); 3.33-3.50 (m, 6H, 2 N—CH₂+1 N/S—CH₂); 10.06 (bs, 2H, 2 HCl salts); 11.03 (bs, 1H, NH). M/Z (M+H)⁺: 242.2.

### Example 174: 6-chloro-2-((3-(1-methylpyrrolidin-2-yl)propyl)thio)-1,4-dihydroquinazoline dihydrochloride

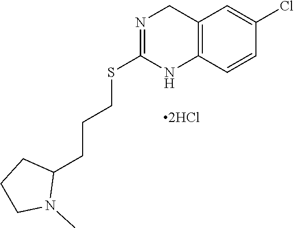 [SMILES: CN1CCCC1CCCSC1=NCc2cc(Cl)ccc2N1]

Crude example 174 was obtained by filtration of the reaction mixture. The solid was passed through an ISOLUTE® SCX-2 cartridge (MeOH, then NH₃1 N in MeOH). To a solution of the resulting residue in DCM (10 mL) was added HCl in Et₂O (2.0 equiv) and, after evaporation to dryness, the crude was purified by flash chromatography (DCM 100% to DCM/MeOH 70:30), then further purified by preparative HPLC (column B, H₂O+0.1% HCOOH/MeCN+0.1% HCOOH 90:10) and freeze-dried with 1 N aqueous HCl (2.0 equiv) to obtain a white solid (77 mg, 33%).

¹H-NMR (D₂O, 300 MHz) δ: 1.60-1.91 (m, 4H, 2 CH₂); 1.91-2.28 (m, 3H, CHₐH_{b}+CH₂); 2.30-2.42 (m, 1H, CHₐH_{b}); 2.90 (s, 3H, N—CH₃); 3.09-3.18 (m, 1H, N—CHₐH_{b}); 3.22-3.39 (m, 3H, N-CHₐH_{b}+S-CH₂); 3.63-3.71 (m, 1H, N—CH); 4.72 (s, 2H, N—CH₂—Ar); 6.99 (d, J8.6 Hz, 1H, Ar); 7.23 (d, J2.1 Hz, 1H, Ar); 7.35 (dd, J8.6, 2.1 Hz, 1H, Ar). M/Z (M[³⁵Cl]+H)⁺: 324.2.

### Example 175: 2-((4-(pyrrolidin-1-yl)butyl)thio)-4,5-dihydro-1H-imidazole dihydrochloride

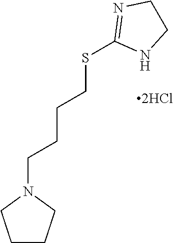 [SMILES: C(CCN1CCCC1)CSC1=NCCN1]

Crude example 175 was obtained by filtration of the reaction mixture. The solid was passed through an ISOLUTE® SCX-2 cartridge (MeOH, then NH₃1 N in MeOH). The resulting crude was purified by preparative HPLC (column B, H₂O+0.1% HCOOH) and freeze-dried with 1 N aqueous HCl (5.0 equiv) to obtain a white solid (70 mg, 34%).

¹H-NMR (D₂O, 300 MHz) δ: 1.78-1.95 (m, 4H, 2 CH₂); 1.97-2.09 (m, 2H, CH₂); 2.10-2.23 (m, 2H, CH₂); 3.05-3.14 (m, 2H, N—CH₂); 3.20-3.27 (m, 4H, 2 N—CH₂); 3.64-3.71 (m, 2H, S—CH₂); 3.96 (s, 4H, 2 N—CH₂). M/Z (M+H)⁺: 228.0.

### Example 176: 2-((4-(1H-imidazol-1-yl)butyl)thio)-6-chloro-1,4-dihydroquinazoline dihydrochloride

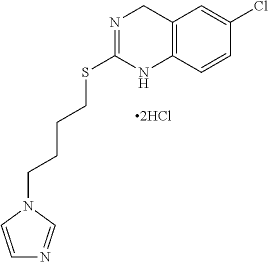 [SMILES: Clc1ccc2c(c1)CN=C(SCCCCn1ccnc1)N2]

Crude example 176 was obtained by filtration of the reaction mixture. The solid was passed through an ISOLUTE® SCX-2 cartridge (MeOH, then NH₃ 3.5 N in MeOH), purified by flash chromatography (KPNH, CyHex 100% to CyHex/EtOAc 0:100), then further purified by preparative HPLC (column B, H₂O+0.1% HCOOH/MeCN+0.1% HCOOH 100:0 to 90:10) and freeze-dried with 1 N aqueous HCl (5.0 equiv) to obtain a white solid (86 mg, 26%). ¹H-NMR (DMSO-d₆, 300 MHz) δ: 1.56-1.66 (m, 2H, CH₂); 1.96 (quint, J7.3 Hz, 2H, CH₂); 3.52 (t, J7.3 Hz, 2H, S—CH₂); 4.27 (t, J7.3 Hz, 2H, N—CH₂—Im); 4.67 (s, 2H, N—CH₂—Ar); 7.28-7.33 (m, 1H, Ar); 7.37-7.41 (m, 2H, 2 Ar); 7.69 (t, J 1.7 Hz, 1H, Ar); 7.83 (t, J1.7 Hz, 1H, Ar); 9.24-9.25 (m, 1H, Ar); HCl salt signals and NH signal not observed. M/Z (M[³⁵Cl]+H)⁺: 321.1.

### Example 177: 6-chloro-2-((2-(1-methylpyrrolidin-3-yl)ethyl)thio)-1,4-dihydroquinazoline dihydrochloride

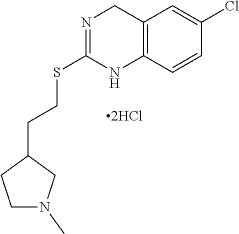 [SMILES: CN1CCC(CCSC2=NCc3cc(Cl)ccc3N2)C1]

Crude example 177 was obtained by filtration of the reaction mixture. The solid was passed through an ISOLUTE® SCX-2 cartridge (MeOH, then NH₃1 N in MeOH). To a solution of the resulting residue in DCM (10 mL) was added HCl in Et₂O (2.0 equiv) and, after evaporation to dryness, the crude was purified by flash chromatography (DCM 100% to DCM/MeOH 70:30), then further purified by preparative HPLC (column B, H₂O+0.1% HCOOH/MeCN+0.1% HCOOH 90:10) and freeze-dried with 1 N aqueous HCl (5.0 equiv) to obtain a white solid (52 mg, 23%).

¹H-NMR (D₂O, 300 MHz) δ: 1.62-1.79 (m, 0.5H, one rotamer of CH—CH₂); 1.80-2.05 (m, 2.5H, other rotamer of CH—CH₂+CH₂); 2.19-2.46 (m, 1H, one rotamer of CHₐH_{b}+one rotamer of CHₐH_{b}); 2.46-2.61 (m, 0.5H, other rotamer of CHₐH_{b}); 2.64-2.85 (m, 1H, other rotamer of CHₐH_{b}+one rotamer of N—CHₐH_{b}); 2.87-2.96 (m, 3H, N—CH₃); 3.04-3.40 (m, 4H, other rotamer of N-CHₐH_{b}+one rotamer of N-CHₐH_{b}+N/S-CHₐH_{b}+N/S—CH₂); 3.60-3.89 (m, 1.5H, other rotamer of N-CHₐH_{b}+N/S—CHₐH_{b}); 4.71 (s, 2H, N—CH₂—Ar); 7.00 (d, J 8.6 Hz, 1H, Ar); 7.23 (d, J 2.2 Hz, 1H, Ar); 7.35 (dd, J 8.6, 2.2 Hz, 1H, Ar). M/Z (M[³⁵Cl]+H)⁺: 310.1.

### Example 178: 2-((4-(pyrrolidin-1-yl)butyl)thio)-4,5,6,7-tetrahydro-1H-1,3-diazepine dihydrochloride

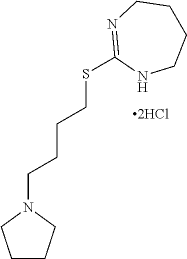 [SMILES: C1CCNC(SCCCCN2CCCC2)=NC1]

Crude example 178 was obtained by filtration of the reaction mixture. The solid was passed through an ISOLUTE® SCX-2 cartridge (MeOH, then NH₃ 1 N in MeOH). The resulting crude was purified by flash chromatography (DCM 100% to DCM/MeOH 70:30), then further purified by preparative HPLC (column B, H₂O+0.1% HCOOH) and freeze-dried with 1 N aqueous HCl (5.0 equiv) to obtain a colorless oil (44 mg, 27%).

¹H-NMR (DMSO-d₆, 300 MHz) δ: 1.60-2.11 (m, 12H, 6 CH₂); 2.88-2.98 (m, 2H, N—CH₂); 3.08-3.15 (m, 2H, N—CH₂); 3.27 (t, J7.2 Hz, 2H, N—CH₂); 3.39-3.53 (m, 6H, 2 N-CH₂+S-CH₂); 9.93 (bs, 2H, HCl salt); 10.95 (bs, 1H, NH). M/Z (M+H)⁺: 256.2.

### Example 179: 5,5-dimethyl-2-((4-(pyrrolidin-1-yl)butyl)thio)-1,4,5,6-tetrahydropyrimidine dihydrochloride

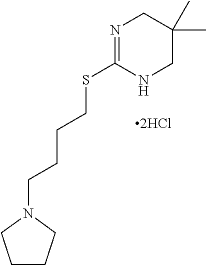 [SMILES: CC1(C)CN=C(SCCCCN2CCCC2)NC1]

Crude example 179 was obtained by filtration of the reaction mixture. The filtrate was concentrated to dryness and passed through an ISOLUTE® SCX-2 cartridge (MeOH, then NH₃1 N in MeOH). The resulting residue was dissolved in DCM (30 mL), washed with water (30 mL) and extracted with 1 N aqueous HCl. The acidic aqueous layer was basified with an aqueous saturated solution of K₂CO₃ (30 mL) and was extracted with DCM (3×20 mL). The combined organic layers were dried over magnesium sulfate and concentrated to dryness, then purified by flash chromatography (KPNH, CyHex 100% to CyHex/EtOAc 0:100). The product was dissolved in MeCN (1 mL) and HCl 2 N in Et₂O (5 mL) was added. The mixture was concentrated to dryness and triturated in MeCN (2×2 mL) to obtain a white solid (66 mg, 40%).

¹H-NMR (DMSO-d₆, 300 MHz) δ: 0.97 (s, 6H, 2 CH₃); 1.61-2.06 (m, 8H, 4 CH₂); 2.83-3.02 (m, 2H, N—CH₂); 3.02-3.22 (m, 6H, 3 N—CH₂); 3.26-3.31 (m, 2H, N—CH₂); 3.42-3.62 (m, 2H, S—CH₂); 10.12 (bs, 2H, NH+HCl salt); 10.94 (bs, 1H, HCl salt). M/Z (M+H)⁺: 270.2.

### Example 180: 2′-((4-(pyrrolidin-1-yl)butyl)thio)-1′H-spiro[cyclopropane-1,4′-quinazoline] dihydrochloride

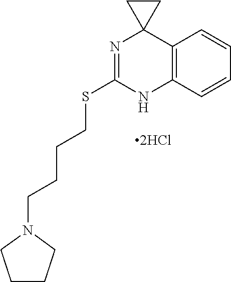 [SMILES: c1ccc2c(c1)NC(SCCCCN1CCCC1)=NC21CC1]

Crude example 180 was obtained by filtration of the reaction mixture. The solid was passed through an ISOLUTE® SCX-2 cartridge (MeOH, then NH₃3.5 N in MeOH). To a solution of the residue in DCM (10 mL) was added HCl in Et₂O (5.0 equiv) and, after evaporation to dryness, the crude was purified by flash chromatography (DCM 100% to DCM/MeOH 70:30), then further purified by preparative HPLC (column B, H₂O+0.1% HCOOH/MeCN+0.1% HCOOH 90:10) and freeze-dried with 1 N aqueous HCl (5.0 equiv) to obtain a white solid (63 mg, 31%).

¹H-NMR (DMSO-d₆, 300 MHz) δ: 1.24-1.29 (m, 2H, CH₂); 1.44-1.53 (m, 2H, CH₂); 1.65-1.98 (m, 8H, 4 CH₂); 2.86-3.02 (m, 2H, N—CH₂); 3.10-3.17 (m, 2H, N—CH₂); 3.44-3.51 (m, 4H, S—CH₂+N-CH₂); 6.86-6.89 (m, 1H, Ar); 7.14-7.29 (m, 3H, 3 Ar); 10.66-10.83 (m, 2H, HCl salts signals); 12.56 (bs, 1H, NH). M/Z (M+H)⁺: 316.1.

### Example 181: 5-benzyl-2-((4-(pyrrolidin-1-yl)butyl)thio)-4,5-dihydro-1H-imidazole dihydrochloride

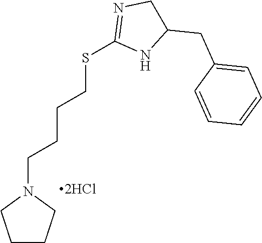 [SMILES: c1ccc(CC2CN=C(SCCCCN3CCCC3)N2)cc1]

Crude example 181 was obtained by filtration of the reaction mixture. The solid was passed through an ISOLUTE® SCX-2 cartridge (MeOH, then NH₃3.5 N in MeOH). The residue was taken-up in 1 N aqueous HCl (5 mL) and washed with DCM (3×5 mL). The resulting aqueous phase was freeze-dried, purified by preparative HPLC (column B, H₂O+0.1% HCOOH/MeCN+0.1% HCOOH 90:10) and freeze-dried with 1 N aqueous HCl (5.0 equiv) to obtain a yellow oil (183 mg, 75%).

¹H-NMR (DMSO-d6,300 MHz) δ: 1.58-2.06 (m, 8H, 4 CH₂); 2.84-3.02 (m, 4H, Ar—CH₂+N-CH₂); 3.07-3.14 (m, 2H, N—CH₂); 3.28 (t, J7.2 Hz, 2H, N—CH₂); 3.41-3.53 (m, 2H, S—CH₂); 3.58 (dd, J10.9, 6.8 Hz, 1H, N—CHₐH_{b}); 3.87 (t, J10.9 Hz, 1H, N—CHₐH_{b}); 4.53-4.62 (m, 1H, N—CH); 7.24-7.37 (m, 5H, 5 Ar); 10.43 (bs, 1H, HCl salt); 10.83 (bs, 1H, HCl salt); 11.13 (bs, 1H, NH). M/Z (M+H)⁺: 318.3.

### Example 182: 2-((2-(pyrrolidin-1-yl)ethyl)thio)-4,5-dihydro-1H-benzo[d][1,3]diazepine dihydrochloride

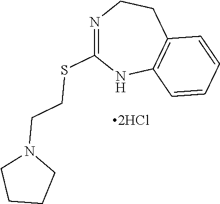 [SMILES: c1ccc2c(c1)CCN=C(SCCN1CCCC1)N2]

Crude example 182 was obtained by elution of the reaction mixture through an ISOLUTE® SCX-2 cartridge (MeOH, then NH₃3.5 N in MeOH). To a solution of the resulting residue in DCM (10 mL) was added HCl in Et₂O (5.0 equiv) and, after evaporation to dryness, the crude was purified by preparative HPLC (column B, H₂O+0.1% HCOOH) and freeze-dried with 1 N aqueous HCl (5.0 equiv) to obtain a yellow varnish (110 mg, 70%).

¹H-NMR (DMSO-d₆, 300 MHz) δ: 1.87-2.01 (m, 4H, 2 CH₂); 2.99-3.12 (m, 2H, Ar—CH₂); 3.18-3.20 (m, 2H, N—CH₂); 3.47-3.62 (m, 4H, N—CH₂+N/S—CH₂); 3.72-3.80 (m, 4H, N—CH₂+N/S—CH₂); 7.18-7.23 (m, 1H, Ar); 7.28-7.35 (m, 2H, 2 Ar); 7.57 (d, J7.7 Hz, 1H, Ar); 11.09-11.16 (m, 2H, 2 HCl salt); 11.93 (bs, 1H, NH). M/Z (M+H)⁺: 276.2.

### Example 183: 5-(4-methoxybenzyl)-5-methyl-2-((2-(pyrrolidin-1-yl)ethyl)thio)-4,5-dihydro-1H-imidazole dihydrochloride

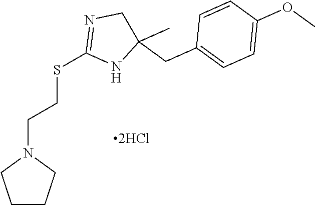 [SMILES: COc1ccc(CC2(C)CN=C(SCCN3CCCC3)N2)cc1]

Crude example 183 was obtained by addition of MeOH (5 mL) to the reaction mixture, followed by elution of the resulting solution through an ISOLUTE® SCX-2 cartridge (MeOH, then NH₃3.5 N in MeOH). The resulting crude was purified by flash chromatography (KPNH, CyHex 100% to CyHex/EtOAc 50:50). To a solution of the residue in DCM (10 mL) was added HCl in Et₂O (5.0 equiv) and, after evaporation to dryness, the resulting crude was purified by preparative HPLC (column B, H₂O+0.1% HCOOH/MeCN+0.1% HCOOH 95:05) and freeze-dried with 1 N aqueous HCl (5.0 equiv) to obtain a yellow varnish (81 mg, 47%).

¹H-NMR (DMSO-d₆, 300 MHz) δ: 1.47 (s, 3H, CH₃); 1.81-2.07 (m, 4H, 2 CH₂); 2.81 (A part of AB system, J13.8 Hz, 1H, Ar—CHₐH_{b}); 2.92 (B part of AB system, J13.8 Hz, 1H, Ar—CHₐH_{b}); 2.98-3.11 (m, 2H, N—CH₂); 3.19-3.43 (m, 2H, N—CH₂); 3.49-3.63 (m, 5H, S—CH₂+N-CH₂+N-CHₐH_{b} of imidazoline); 3.74 (s, 3H, O—CH₃); 3.80 (d, J 11.0 Hz, 1H, N—CHₐH_{b} of imidazoline); 6.88-6.93 (m, 2H, 2 Ar); 7.23-7.28 (m, 2H, 2 Ar); 10.22 (s, 1H, HCl salt); 10.96 (bs, 1H, NH); 11.02 (s, 1H, HCl salt). M/Z (M+H)⁺: 334.3.

### Example 184: 2-((2-(pyrrolidin-1-yl)ethyl)thio)-1,4,4a,5,6,7,8,8a-octahydroquinazoline dihydrochloride

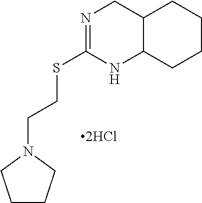 [SMILES: C1CCC2NC(SCCN3CCCC3)=NCC2C1]

Crude example 184 was obtained by addition of MeOH (5 mL) to the reaction mixture, followed by elution of the resulting solution through an ISOLUTE® SCX-2 cartridge (MeOH, then NH₃3.5 N in MeOH). The resulting crude was purified by flash chromatography (KPNH, CyHex 100% to CyHex/EtOAc 50:50). To a solution of the resulting residue in DCM (10 mL) was added HCl in Et₂O (5.0 equiv) and, after evaporation to dryness, the residue was purified by preparative HPLC (column B, H₂O+0.1% HCOOH/MeCN+0.1% HCOOH 95:5) and freeze-dried with 1 N aqueous HCl (2.0 equiv) to obtain a colorless oil (55 mg, 33%).

¹H-NMR (DMSO-d₆, 300 MHz) δ: 0.95-2.14 (m, 13H, CH+6 CH₂); 3.00-3.73 (m, 11H, 4 N-CH₂+S-CH₂+N-CH); 10.20-10.38 (m, 2H, 2 HCl salts); 11.24 (bs, 1H, NH). M/Z (M+H)⁺: 268.2.

### Example 185: 5-((4-(pyrrolidin-1-yl)butyl)thio)-4,6-diazaspiro[2.4]hept-5-ene dihydrochloride

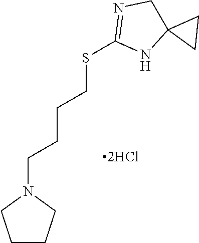 [SMILES: C(CCN1CCCC1)CSC1=NCC2(CC2)N1]

Crude example 185 was obtained by dilution of the reaction mixture with MeOH followed by elution of the resulting solution through an ISOLUTE® SCX-2 cartridge (MeOH, then NH₃ 3.5 N in MeOH). To a solution of the resulting residue in DCM (25 mL) was added HCl in Et₂O (5.0 equiv) and, after evaporation to dryness, the crude was purified by preparative HPLC (column B, H₂O+0.1% HCOOH) and freeze-dried with 1 N aqueous HCl (2.0 equiv). The residue was dissolved in water (10 mL), washed with DCM (2×10 mL) and freeze-dried. The residue was then taken-up in saturated aqueous NaHCO₃ (8 mL) and extracted with DCM (2×10 mL). The combined organic layers were filtered through a hydrophobic cartridge, acidified with HCl in Et₂O (5 equiv) and concentrated to dryness to obtain a colorless oil (24 mg, 9%).

¹H-NMR (DMSO-d₆, 300 MHz) δ: 0.89-0.93 (m, 2H, CH₂); 1.10-1.14 (m, 2H, CH₂); 1.66-2.03 (m, 8H, 4 CH₂); 2.88-2.99 (m, 2H, N—CH₂); 3.08-3.15 (m, 2H, N—CH₂); 3.30 (t, J7.2 Hz, 2H, N—CH₂); 3.44-3.51 (m, 2H, N/S—CH₂); 3.92 (s, 2H, N/S—CH₂); 10.45 (bs, 1H, HCl salt); 10.76 (bs, 1H, NH); 10.91 (bs, 1H, HCl salt). M/Z (M+H)⁺: 254.2.

### Example 186: 3-((2-(pyrrolidin-1-yl)ethyl)thio)-2,5-dihydro-1H-benzo[e][1,3]diazepine dihydrochloride

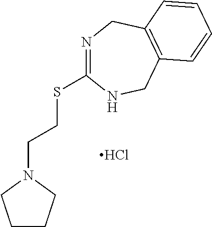 [SMILES: c1ccc2c(c1)CN=C(SCCN1CCCC1)NC2 ; Cl]

Crude example 186 was obtained by addition of MeOH (20 mL) to the reaction mixture, followed by filtration and elution of the filtrate through an ISOLUTE® SCX-2 cartridge (MeOH, then NH₃ 3.5 N in MeOH). To a solution of the resulting residue in DCM (10 mL) was added HCl in Et₂O (5.0 equiv) and, after evaporation to dryness, the crude was purified by preparative HPLC (column B, H₂O+0.1% HCOOH/MeCN+0.1% HCOOH 95:05) and freeze-dried with 1 N aqueous HCl (2.0 equiv) to obtain a yellow varnish (98 mg, 31%).

¹H-NMR (DMSO-d₆, 300 MHz) δ: 1.80-2.05 (m, 4H, 2 CH₂); 2.98-3.09 (m, 2H, N—CH₂); 3.37-3.44 (m, 2H, N—CH₂); 3.47-3.56 (m, 2H, N/S—CH₂); 3.60-3.65 (m, 2H, N/S—CH₂); 4.78 (s, 2H, N—CH₂—Ar); 4.79 (s, 2H, N—CH₂—Ar); 7.36-7.43 (m, 4H, 4 Ar); 10.50 (m, 2H, 2 HCl salts); 11.00 (bs, 1H, NH). M/Z (M+H)⁺: 276.3.

### Example 187: 5-((2-(pyrrolidin-1-yl)ethyl)thio)-4,6-diazaspiro[2.4]hept-5-ene dihydrochloride

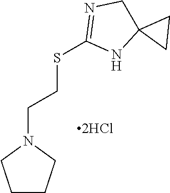 [SMILES: C1CCN(CCSC2=NCC3(CC3)N2)C1]

Example 187 was isolated as a colorless oil (175 mg, 63%), by addition of MeOH (3 mL) to the reaction mixture, followed by elution of the resulting solution through an ISOLUTE® SCX-2 cartridge (MeOH, then NH₃3.5 N in MeOH), then purification of the resulting residue by flash chromatography (KPNH, CyHex 100% to CyHex/EtOAc 50:50), then purification by preparative HPLC (column B, H₂O+0.1% HCOOH), then freeze-drying with 1 N aqueous HCl.

¹H-NMR (D₂O, 300 MHz) δ: 0.96-1.01 (m, 2H, CH₂); 1.15-1.20 (m, 2H, CH₂); 1.97-2.25 (m, 4H, 2 CH₂); 3.13-3.21 (m, 2H, N—CH₂); 3.54-3.65 (m, 4H, 2 N—CH₂); 3.71-3.78 (m, 2H, S—CH₂); 4.02 (s, 2H, N—CH₂). M/Z (M+H)⁺: 226.2.

### Example 188: 2-((pyridin-4-ylmethyl)thio)-4,5-dihydro-1H-benzo[d][1,3]diazepine dihydrochloride

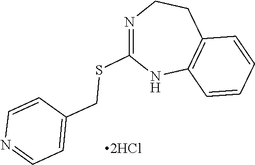 [SMILES: c1ccc2c(c1)CCN=C(SCc1ccncc1)N2]

Crude example 188 was obtained by hydrolysis of the reaction mixture with saturated aqueous NaHCO₃ (30 mL) followed by extraction with EtOAc (50 mL). The organic layer was dried over magnesium sulfate then concentrated to dryness. The crude was purified by flash chromatography (CyHex 100% to EtOAc 100% to EtOAc/MeOH 80:20). To a solution of the resulting residue in DCM (5 mL) was added HCl in Et₂O (1 mL) and, after evaporation to dryness, the resulting crude was purified by preparative HPLC (column D, H₂O+0.1% HCOOH/MeCN+0.1% HCOOH 100:0 to 0:100) and alkalized with saturated aqueous NaHCO₃ (30 mL), then extracted with EtOAc (2×50 mL). The combined organic extracts were dried over magnesium sulfate then concentrated to dryness. The residue was dissolved in DCM (5 mL) then HCl 2 N in Et₂O (1 mL) was added and the mixture was concentrated to dryness, dissolved in water and filtrated. The filtrate was freeze-dried in water to obtain a white solid (50 mg, 26%).

¹H-NMR (DMSO-d₆, 300 MHz) δ: 2.98-3.06 (m, 2H, CH₂); 3.60-3.62 (m, 2H, N—CH₂); 4.94 (s, 2H, S—CH₂); 7.15-7.32 (m, 3H, 3 Ar); 7.43-7.45 (m, 1H, Ar); 7.88-7.90 (m, 2H, 2 Ar); 8.79-8.81 (m, 2H, 2 Ar); 11.23 (bs, 1H, HCl salt); 11.91 (bs, 1H, NH); other HCl signal not observed. M/Z (M+H)⁺: 270.2.

### Example 189: 3-((pyridin-4-ylmethyl)thio)-2,5-dihydro-1H-benzo[e][1,3]diazepine dihydrochloride

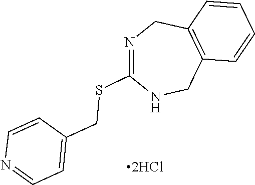 [SMILES: c1ccc2c(c1)CN=C(SCc1ccncc1)NC2]

Crude example 189 was obtained by filtration of the reaction mixture, followed by washing to the solid with MeCN (2 mL). The solid was taken-up in saturated aqueous NaHCO₃ (20 mL) and extracted with DCM (3×20 mL). The combined organics extracts were dried over magnesium sulfate and concentrated to dryness. The crude was then purified by flash chromatography (KPNH, CyHex 100% to CyHex/EtOAc 0:100). The resulting residue was dissolved in DCM (5 mL) and extracted with 1 N aqueous HCl (20 mL). The aqueous layer was washed with DCM (2×5 mL) and freeze-dried to obtain a brown solid (112 mg, 58%).

¹H-NMR (DMSO-d₆, 300 MHz) δ: 4.67-4.68 (m, 4H, 2 N—CH₂); 4.87 (s, 2H, S—CH₂); 7.28-7.38 (m, 4H, 4 Ar); 7.90-7.92 (m, 2H, 2 Ar); 8.73-8.75 (m, 2H, 2 Ar); 10.66-10.94 (m, 2H, NH+HCl salt); 2nd HCl signal not observed. M/Z (M+H)⁺: 270.2.

### Example 190: 2-((3-(pyrrolidin-1-yl)propyl)thio)-4,5-dihydro-3H-benzo[d][1,3]diazepine dihydrochloride

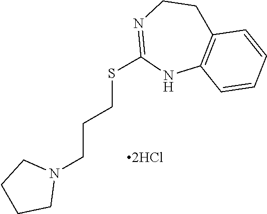 [SMILES: c1ccc2c(c1)CCN=C(SCCCN1CCCC1)N2 ; "·2HCl"]

Crude example 190 was obtained by hydrolysis of the reaction mixture with saturated aqueous NaHCO₃ (50 mL) followed by extraction with EtOAc (3×10 mL), drying over magnesium sulfate and concentration to dryness. The crude was purified by flash chromatography (KPNH, CyHex 1000% to CyHex/EtOAc 50:50). The residue was diluted in dioxane (5 mL), then 4 N HCl in dioxane (10 equiv) was added and the mixture was concentrated to dryness. The residue was further purified by preparative HPLC (column D, H₂O+0.1% HCOOH/MeCN+0.1% HCOOH 100:0 to 0:100), then freeze-dried with 1 N aqueous HCl to obtain a colorless oil (99 mg, 61%).

¹H-NMR (DMSO-d₆, 300 MHz) δ: 1.81-2.04 (m, 4H, 2 CH₂); 2.06-2.16 (m, 2H, CH₂); 2.90-3.05 (m, 2H, N—CH₂); 3.18-3.30 (m, 4H, Ar—CH₂+N-CH₂); 3.49-3.52 (m, 4H, 2 N—CH₂); 3.68-3.77 (m, 2H, S—CH₂); 7.17-7.22 (m, 1H, Ar); 7.28-7.34 (m, 2H, 2 Ar); 7.49-7.52 (m, 1H, Ar); 10.93 (bs, 1H, HCl salt); 11.09 (bs, 1H, HCl salt); 11.77 (bs, 1H, NH). M/Z (M+H)⁺: 290.1.

### Example 191: 2-((2-(3,4-dihydroquinolin-1(2H)-yl)ethyl)thio)-4,5-dihydro-1H-benzo[d][1,3]diazepine hydrochloride

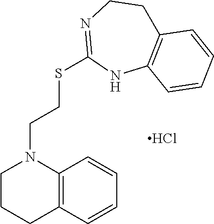 [SMILES: c1ccc2c(c1)CCN=C(SCCN1CCCc3ccccc31)N2 ; Cl]

Crude example 191 was obtained by elution of the reaction mixture through an ISOLUTE® SCX-2 cartridge (MeOH, then NH₃3.5 N in MeOH). To a solution of the resulting residue in DCM (10 mL) was added HCl in Et₂O (5.0 equiv) and, after evaporation to dryness, the crude was purified by preparative HPLC (column C, H₂O+0.1% HCOOH/MeCN+0.1% HCOOH 70:30), then freeze-dried with 1 N aqueous HCl to obtain a pale orange solid (120 mg, 23% over 2 steps).

¹H-NMR (DMSO-d₆, 300 MHz) δ: 1.81 (quint, J6.2 Hz, 2H, CH₂); 2.65 (t, J6.2 Hz, 2H, Ar—CH₂); 2.99-3.02 (m, 2H, Ar—CH₂); 3.29-3.31 (m, 2H, N—CH₂); 3.40-3.47 (m, 2H, N/S—CH₂); 3.51-3.55 (m, 4H, 2 N/S—CH₂); 6.49 (t, J7.2 Hz, 1H, Ar); 6.68-6.73 (m, 1H, Ar); 6.84-6.87 (m, 1H, Ar); 6.90-6.96 (m, 1H, Ar); 7.03-7.12 (m, 1H, Ar); 7.17-7.27 (m, 3H, 3 Ar); 11.42 (bs, 1H, NH); HCl salt signal not observed. M/Z (M+H)⁺: 338.3.

### Example 192: 2-((2-(indolin-1-yl)ethyl)thio)-4,5-dihydro-1H-benzo[d][1,3]diazepine hydrochloride

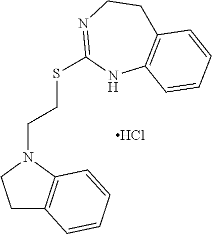 [SMILES: c1ccc2c(c1)CCN=C(SCCN1CCc3ccccc31)N2 ; Cl]

Crude example 192 was obtained by elution of the reaction mixture through an ISOLUTE® SCX-2 cartridge (MeOH, then NH₃3.5 N in MeOH). To a solution of the resulting residue in DCM (10 mL) was added HCl in Et₂O (5.0 equiv) and, after evaporation to dryness, the crude was purified by preparative HPLC (column C, H₂O+0.1% HCOOH/MeCN+0.1% HCOOH 70:30), then freeze-dried with 1 N aqueous HCl to obtain a pale orange solid (105 mg, 19% over 2 steps).

¹H-NMR (DMSO-d₆, 300 MHz) δ: 2.70 (t, J8.3 Hz, 2H, Ar—CH₂); 2.92-2.95 (m, 2H, Ar—CH₂); 3.35 (t, J8.3 Hz, 2H, N—CH₂); 3.42 (t, J6.2 Hz, 2H, N/S—CH₂); 3.50-3.54 (m, 2H, N—CH₂); 3.67 (t, J6.2 Hz, 2H, N/S—CH₂); 6.55-6.62 (m, 2H, 2 Ar); 6.96-7.01 (m, 2H, 2 Ar); 7.13-7.24 (m, 2H, 2 Ar); 7.26-7.31 (m, 1H, Ar); 7.44 (dd, J8.1, 0.8 Hz, 1H, Ar); 10.86 (m, 1H, NH); 11.65 (s, 1H, HCl salt). M/Z (M+H)⁺: 324.2.

### Example 193: 3-((pyridin-3-ylmethyl)thio)-2,5-dihydro-1H-benzo[e][1,3]diazepine

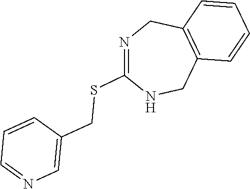 [SMILES: c1cncc(CSC2=NCc3ccccc3CN2)c1]

Crude example 193 was obtained by filtration of the reaction mixture, followed by washing to the solid with MeCN (5 mL). The solid was taken-up in saturated aqueous NaHCO₃ (20 mL) and extracted with DCM (3×30 mL). The combined organics extracts were dried over magnesium sulfate and concentrated to dryness. The crude was then purified by flash chromatography (KPNH, CyHex 100% to CyHex/EtOAc 0:100). The resulting residue was dissolved in DCM (5 mL) and extracted with 1 N aqueous HCl (10 mL). The aqueous layer was freeze-dried, the residue was purified by preparative HPLC (column D, H₂O+0.1% HCOOH) and alkalized with saturated aqueous NaHCO₃ (10 mL), then extracted with EtOAc (100 mL). The combined organic extracts were dried over magnesium sulfate then concentrated to dryness. The residue was freeze-dried in MeCN and water to obtain a pale brown gum (34 mg, 32%).

¹H-NMR (DMSO-d₆, 300 MHz) δ: 4.03 (s, 2H, S—CH₂); 4.23-4.91 (m, 4H, 2 N—CH₂); 6.90 (bs, 1H, NH); 7.13-7.40 (m, 5H, 5 Ar); 7.65-7.69 (m, 1H, Ar); 8.37-8.39 (m, 1H, Ar); 8.47-8.48 (m, 1H, Ar). M/Z (M+H)⁺: 270.2.

### Example 194: 3-((3-(pyrrolidin-1-yl)propyl)thio)-2,5-dihydro-1H-benzo[e][1,3]diazepine dihydrochloride

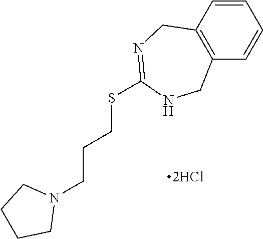 [SMILES: c1ccc2c(c1)CN=C(SCCCN1CCCC1)NC2]

Crude example 194 was obtained by dilution of the reaction mixture in MeOH (8 mL), and elution of the resulting solution through an ISOLUTE® SCX-2 cartridge (MeOH, then NH₃ 3.5 N in MeOH). The resulting crude was purified by flash chromatography (KPNH, CyHex 100% to CyHex/EtOAc 40:60). To a solution of the resulting residue in DCM (10 mL) was added HCl in Et₂O (5.0 equiv) and, after evaporation to dryness, the product was freeze-dried in water to obtain a pale rose solid (111 mg, 68%).

¹H-NMR (DMSO-d₆, 300 MHz) δ: 1.82-2.02 (m, 6H, 3 CH₂); 2.86-2.96 (m, 2H, N/S—CH₂); 3.14-3.20 (m, 2H, N/S—CH₂); 3.33 (t, J7.0 Hz, 2H, N/S—CH₂); 3.41-3.50 (m, 2H, N/S—CH₂); 4.78-4.80 (m, 4H, 2 N—CH₂—Ar); 7.36-7.43 (m, 4H, 4Ar); 10.32 (bs, 2H, 2 HCl salts); 10.87 (bs, 1H, NH). M/Z (M+H)⁺: 290.2.

### Example 195: 3-((2-(indolin-1-yl)ethyl)thio)-2,5-dihydro-1H-benzo[e][1,3]diazepine

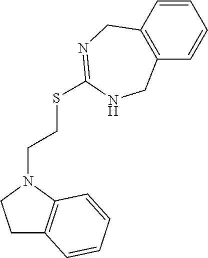 [SMILES: c1ccc2c(c1)CN=C(SCCN1CCc3ccccc31)NC2]

Crude example 195 was obtained by elution of the reaction mixture through an ISOLUTE® SCX-2 cartridge (MeOH, then NH₃3.5 N in MeOH). The crude was purified by preparative HPLC (column C, H₂O+0.1% HCOOH/MeCN+0.1% HCOOH 75:25), then freeze-dried with water. The residue was taken-up in saturated aqueous NaHCO₃ (40 mL) then extracted with EtOAc (2×40 mL). The combined organic layers were dried over magnesium sulfate then concentrated to dryness. The residue was purified by flash chromatography (KPNH, CyHex 100% to CyHex/EtOAc 70:30) and freeze-dried in water and MeCN to obtain a white solid (41 mg, 23% over 2 steps).

¹H-NMR (DMSO-d₆, 300 MHz) δ: 2.85 (t, J8.3 Hz, 2H, Ar—CH₂); 2.92 (m, 2H, N/S—CH₂); 3.15 (m, 2H, N/S—CH₂); 3.25-3.29 (m, 2H, N/S—CH₂); 4.35 (bs, 2H, N—CH₂—Ar); 4.70 (bs, 2H, N—CH₂—Ar); 6.48-6.56 (m, 2H, 2 Ar); 6.84 (bs, 1H, NH); 6.94-7.00 (m, 2H, 2 Ar); 7.20-7.26 (m, 4H, 4 Ar). M/Z (M+H)⁺: 324.3.

### Example 196: 3-(((1,4-dihydroquinazolin-2-yl)thio)methyl)-6-(4-fluorobenzyl)-5,6-dihydroimidazo[2,1-b]thiazole dihydrochloride

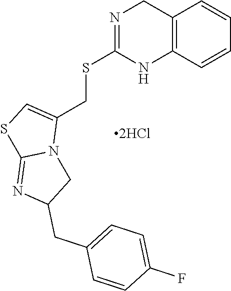 [SMILES: Fc1ccc(CC2CN3C(CSC4=NCc5ccccc5N4)=CSC3=N2)cc1]

Example 196 was isolated by concentration to dryness of the reaction mixture, then hydrolysis with water (20 mL), then washing with EtOAc (2×10 mL), followed by freeze-drying of the resulting aqueous layer to obtain a white solid (210 mg, 96%).

¹H-NMR (DMSO-d₆, 400 MHz) δ: 3.09 (d, J 6.6 Hz, 2H, CH₂—Ar); 4.28 (dd, J10.8, 6.8 Hz, 1H, N—CHₐH_{b}); 4.52 (t, J 10.5 Hz, 1H, N—CHₐH_{b}); 4.65 (bs, 2H, N—CH₂); 4.76-4.93 (m, 2H, S—CH₂); 4.97-5.04 (m, 1H, N—CH); 6.99 (bs, 1H, S—CH); 7.15-7.41 (m, 8H, 8 Ar); 10.05 (bs, 1H, HCl salt); 11.26 (bs, 1H, HCl salt); 12.93 (bs, 1H, NH). M/Z (M+H)⁺: 411.2. Mp: 155-160° C.

### Example 197: 2-((2-cyclopentylethyl)thio)-1,4-dihydroquinazoline hydrochloride

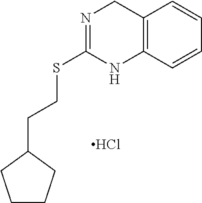 [SMILES: c1ccc2c(c1)CN=C(SCCC1CCCC1)N2 ; Cl]

Crude example 197 was obtained by concentration to dryness of the reaction, followed by elution of the resulting residue through an ISOLUTE® SCX-2 cartridge (DCM, then NH₃3.5 N in MeOH). The crude was then dissolved in 1 N aqueous HCl (10 mL) and washed with DCM (2×5 mL). The resulting aqueous layer was freeze-dried, purified by preparative HPLC (column B, H₂O+0.1% HCOOH/MeCN+0.1% HCOOH 95:5 to 55:45) and freeze-dried with 1 N aqueous HCl (5 equiv) to obtain a white solid (23 mg, 8%).

¹H-NMR (DMSO-d₆, 300 MHz) δ: 1.07-1.19 (m, 2H, CH₂); 1.42-1.60 (m, 4H, 2 CH₂); 1.62-1.69 (m, 2H, CH₂); 1.70-1.81 (m, 2H, CH₂); 1.84-1.93 (m, 1H, CH); 3.35-3.40 (m, 2H, S—CH₂); 4.70 (s, 2H, N—CH₂—Ar); 7.16-7.18 (m, 1H, Ar); 7.23-7.24 (m, 2H, 2 Ar); 7.29-7.36 (m, 1H, Ar); 10.56 (bs, 1H, HCl salt); 12.28 (bs, 1H, NH). M/Z (M+H)⁺: 261.2. Mp: 42-50° C.

The remaining examples of the invention were prepared as described hereinafter.

### Example 198: tert-butyl (S)-3-((4,5-dihydro-1H-benzo[d][1,3]diazepin-2-yl)thio)pyrrolidine-1-carboxylate

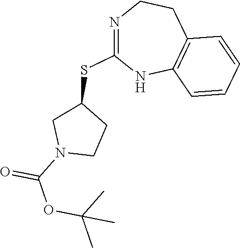 [SMILES: CC(C)(C)OC(=O)N1CC[C@H](SC2=NCCc3ccccc3N2)C1]

To a suspension of intermediate 212 (135 mg, 1.0 equiv) and potassium carbonate (146 mg, 2.5 equiv) in MeCN (3 mL) sparged with argon, was added a solution of (S)-tert-butyl 3-mercaptopyrrolidine-1-carboxylate (129 mg, 1.5 equiv) in MeCN sparged with argon (1 mL). The reaction was heated at 80° C. for 30 h, then the reaction mixture was diluted with water (20 mL) and extracted with EtOAc (30 mL). The organic layer was dried over magnesium sulfate and concentrated to dryness. The crude was purified by flash chromatography (KPNH, CyHex 100% to CyHex/EtOAc 50:50). The residue was dissolved in Et₂O (20 mL) then extracted with 1 N aqueous HCl. The resulting aqueous layer was alkalized with saturated aqueous NaHCO₃ and then extracted with EtOAc (2×20 mL). The resulting organic extracts were dried over magnesium sulfate and concentrated to dryness to obtain a white solid (60 mg, 41%).

¹H-NMR (DMSO-d₆, 300 MHz) δ: 1.40 (s, 9H, (CH₃)₃—C); 1.80-1.96 (m, 1H, CHₐH_{b}); 2.18-2.35 (m, 1H, CHₐH_{b}); 2.90-2.93 (m, 2H, Ar—CH₂); 3.19-3.28 (m, 1H, N—CHₐH_{b}); 3.33-3.51 (m, 4H, 2 N—CH₂); 3.68-3.84 (m, 1H, N—CHₐH_{b}); 4.01-4.16 (m, 1H, S—CH); 6.83-6.88 (m, 1H, Ar); 6.98-7.02 (m, 2H, 2 Ar); 7.07-7.12 (m, 1H, Ar); 7.70 (bs, 1H, NH). M/Z (M+H)⁺: 348.3.

### Example 199: (S)-2-(pyrrolidin-3-ylthio)-4,5-dihydro-3H-benzo[d][1,3]diazepine dihydrochloride

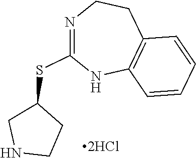 [SMILES: c1ccc2c(c1)CCN=C(S[C@H]1CCNC1)N2]

To a solution of example 198 (150 mg, 1.0 equiv) in 1,4-dioxane (3 mL) was added HCl in dioxane (3.2 mL, 4 M, 30 equiv). The reaction was stirred at 25° C. for 4 h and then concentrated to dryness to obtain a pale-yellow oil which was purified by preparative HPLC (column D, H₂O+0.1% HCOOH/MeCN+0.1% HCOOH 100:0) and freeze-dried with 1 N aqueous HCl to obtain a colorless oil (111 mg, 62%).

¹H-NMR (DMSO-d₆, 300 MHz) δ: 1.98-2.09 (m, 1H, CHₐH_{b}); 2.41-2.50 (m, 1H, CHₐH_{b}); 3.14-3.22 (m, 2H, CH₂—Ar); 3.24-3.37 (m, 3H, N—CH₂+N-CHₐH_{b}); 3.64-3.77 (m, 3H, N—CH₂+N-CHₐH_{b}); 4.53-4.68 (m, 1H, S—CH); 7.19-7.24 (m, 1H, Ar); 7.29-7.35 (m, 2H, 2 Ar); 7.48-7.60 (m, 1H, Ar); 9.52-9.90 (bs, 2H, 2 HCl salts); 11.22 (bs, 1H, NH); 11.98 (bs, 1H, NH). M/Z (M+H)⁺: 248.0.

### Example 200: (S)-2-((1-methylpyrrolidin-3-yl)thio)-4,5-dihydro-3H-benzo[d][1,3]diazepine dihydrochloride

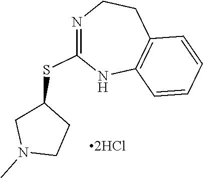 [SMILES: CN1CC[C@H](SC2=NCCc3ccccc3N2)C1]

To a solution of example 199 (130 mg, 1.0 equiv) in formic acid (6 mL) was added formaldehyde (103 μL, 37% aq., 3 equiv). The reaction was heated at 110° C. for 90 min, then water (6 mL) was added and the solution was freeze-dried. The crude residue was purified by preparative HPLC (column D, H₂O+0.1% HCOOH), then freeze-dried with 1 N aqueous HCl to obtain a white gum (77 mg, 58%).

¹H-NMR (D₂O, 300 MHz) δ: 2.15-2.27 (m, 0.6H, one rotamer of CHₐH_{b}); 2.32-2.43 (m, 0.4H, other rotamer of CHₐH_{b}); 2.64-2.77 (m, 0.4H, one rotamer of CHₐH_{b}); 2.83-2.95 (m, 0.6H, other rotamer of CHₐH_{b}); 3.02 (s, 1.8H, one rotamer of N—CH₃); 3.06 (s, 1.2H, other rotamer of N—CH₃); 3.25-3.36 (m, 3H, CH₂+N-CHₐH_{b}); 3.40-3.50 (m, 0.4H, one rotamer of N—CHₐH_{b}); 3.69-3.76 (m, 0.6H, other rotamer of N—CHₐH_{b}); 3.82-3.98 (m, 3.6H, N—CH₂+N-CHₐH_{b}+one rotamer of N—CHₐH_{b}); 4.23-4.30 (m, 0.4H, other rotamer of N—CHₐH_{b}); 4.38-4.48 (m, 0.4H, one rotamer of S—CH); 4.58-4.66 (m, 0.6H, other rotamer of S—CH); 7.23-7.45 (m, 4H, 4 Ar). M/Z (M+H)⁺: 262.2.

### Example 201: Biological Evaluation

Materials and Methods:

A. Immune Cells Preparation

The blood from healthy donors was obtained from “Etablissement Français du Sang” (convention #19/EFS/029), Paris, France.

In vitro experiments were performed using human peripheral blood mononuclear cells (PBMCs) isolated by density centrifugation from peripheral blood leukocyte separation medium, lymphoprep (Stemcell Technologies).

PBMCs were cultured in RPMI 1640 (Sigma-Aldrich, MO, USA) containing 10% heat-inactivated fetal bovine serum (Sigma-Aldrich, MO, USA) at 37° C./5% CO₂.

B. Immune Cells Stimulation

The PBMCs used herein were prepared as described in part A “immune cells preparation”, above. PBMCs were seeded at 4.10⁶/mL. Cells were pre-treated with different concentration of examples according to the invention. Cells were then stimulated with the TLR7/8 agonist resiquimod—R848 (Sigma-Aldrich, MO, USA) at 5 μg/mL.

C. Quantification of Interferon Secretion

To quantify the secretion of functional IFN, a biological assay based on a stable cell line where luciferase reporter gene is controlled by five Interferon-Stimulated Response Elements (twINNE cell line) was used. First, supernatants of R848-stimulated PBMCs (see part B above) were harvested after 24 h of stimulation and frozen at −20° C. for storage. Then, supernatants were dispensed in culture wells of a 96-well plate containing 35.103 twINNE cells per well in Dulbecco's Modified Eagle's Medium supplemented with 10% Foetal Bovine Serum, 100 U/mL Penicillin and 100 μg/mL Streptomycin (1% Pen-Strep) and 1 mM glutamine (Sigma-Aldrich, MO, USA) at 37° C./5% CO₂. After 24 h of culture, luciferase activity was determined by adding 60 μL of Bright-Glo reagents (Promega, Wisconsin, USA) to culture wells and measuring bioluminescence with EnSpire Multimode Plate Reader (PerkinElmer, Massachusetts, USA). When stated, for IC₅₀ calculation, dose-response curves fitted using the nonlin fit (variable slope) analysis in GraphPad Prism software (GraphPad Software, Califronia, USA).

D. BRET Assays

Examples of the present invention were tested for their antagonist activity on human CXCR4 (hCXCR4) receptor transiently over-expressed in HEK-293 T cells. Compounds exert antagonist activity if they decrease the action of CXCL12 on the receptor.

The assay used to measure compound activity is based on BRET (Bioluminescence Resonance Energy Transfer) biosensors and is designed to monitor the plasma membrane translocation of protein that interacts with specific Ga subunit. The specific effector (luciferase tagged: BRET donor) recruited at the membrane will be in close proximity to a plasma membrane anchor (GFP tagged: BRET acceptor) to induce a BRET signal (Hamdan et a1, 2006, Chapter 5, Current Protocols in Neuroscience).

HEK-293 T cells are maintained in Dulbecco's Modified Eagle's Medium supplemented with 10% Foetal Calf Serum, 1% Penicillin/Streptomycin at 37° C./5% CO₂. Cells are co-transfected using polyethylenimine (25 kDa linear) with several DNA plasmids encoding: hCXCR4; Gai2; an intracellular effector fused to luciferase (BRET donor); a plasma membrane effector fused to GFP (BRET acceptor). After transfection, cells are cultured for 48 h at 37° C./5% CO₂. On the day of the assay, cells are detached using trypsin 0.05%, resuspended in assay buffer (1.8 mM CaCl₂, 1 mM MgCl₂, 2.7 mM KCl, 137 mM NaCl, 0.4 mM NaH₂PO₄, 5.5 mM D-Glucose, 11.9 mM NaHCO₃, 25 mM Hepes) and seeded in 384 well plate at a density of 20,000 cells per well. Then, plates are equilibrated 3.5 h at 37° C. before adding compounds. Compounds and luciferase substrate are added to the cells using an automated device (Freedom Evo®, Tecan) and BRET readings are collected on EnVision (PerkinElmer) with specific filters (410 nm BW 80 nm, 515 nm BW 30 nm).

Cells are first incubated for 10 minutes with the compound alone. Then, cells are stimulated by an EC₈₀ CXCL12 concentration for additional 10 minutes and luminescence is recorded. EC₈₀ CXCL12 concentration is the concentration giving 80% of the maximal CXCL12 response. Antagonist activity is evaluated in comparison to basal signals evoked by EC₈₀ CXCL12 alone.

For IC₅₀ determination, a dose-response test is performed using 20 concentrations (ranging over 6 logs) of each compound. Dose-response curves are fitted using the sigmoidal dose-response (variable slope) analysis in GraphPad Prism software (GraphPad Software) and IC₅₀ of antagonist activity is calculated. Dose-response experiments are performed in duplicate, in two independent experiments.

Results:

Effect of Compounds of Formula (I) on Interferon Production by Peripheral Blood Mononuclear Cells (PBMCs) from Healthy Donors

PBMCs from two healthy donors were cultured (as specified in Materials and Methods, part B “immune cells stimulation”) in the presence of different concentrations of various examples according to the invention (see Table 1 below) or IT1t (positive control) and activated by 5 μg/mL of R848. IFN production was quantified (as specified in Materials and Methods, part C “quantification of interferon secretion”) using twINNE reporter cell line. The examples with IC₅₀<32 μM according to the invention showed a higher potency than IT1t to reduce IFN production by activated PBMCs, as detailed in Table 1.

TABLE 1
Effect of various examples according to the invention on IFN production by activated
PBMCs. PBMCs from 2 healthy donors' blood were isolated by Ficoll gradient and cells
were incubated with different concentrations of the respective example or IT1t before
activation with the TLR7 ligand R848 overnight. IFN secretion in the supernatant was
quantified using twINNE reporter cell line. Levels of IFN were measured by quantifying
the luciferase activity induced by the presence of IFN and IC₅₀ of anti-
inflammatory activity were calculated using GraphPad prism software. The IC₅₀ values of all
the examples presented are the calculations of two results over 2 healthy donors.
For IT1t, the IC₅₀ value presented is the mean of at least 7 independent
sets of results over various donors.

| Examples | IC₅₀ |
|---|---|
| 1, 3, 4, 6, 8, 10, 11, 12, 13, 14, 15, 16, 18, 19, 20, 21, 22, 23, 24, 25, 26, | IC₅₀ ≤ 10 μM |
| 27, 28, 29, 30, 31, 32, 33, 34, 35, 36, 37, 38, 39, 40, 41, 42, 43, 45, 46, | |
| 47, 48, 49, 59, 60, 61, 63, 64, 65, 66, 68, 69, 70, 72, 77, 79, 80, 81, 82, | |
| 83, 84, 85, 86, 87, 88, 89, 90, 91, 92, 93, 94, 95, 99, 100, 103, 104, 109, | |
| 110, 111, 112, 113, 114, 116, 117, 118, 119, 120, 121, 122, 123, 124, | |
| 126, 127, 128, 129, 130, 133, 134, 136, 138, 140, 142, 143, 148, 149, | |
| 150, 151, 153, 158, 161, 162, 163, 164, 166, 167, 174, 177, 187, 196, | |
| 200 | |
| 2, 5, 9, 53, 54, 55, 56, 58, 67, 71, 76, 101, 102, 105, 106, 108, 115, 125, | 10 μM < IC₅₀ < 32 μM |
| 131, 132, 137, 139, 141, 145, 146, 152, 156, 157, 159, 165, 168, 176, | |
| 180, 182, 188, 190, 191, 192, 197, 198, 199 | |
| IT1t | 32 μM |
| 7, 17, 44, 50, 51, 52, 57, 73, 74, 75, 78, 96, 97, 98, 135, 144, 147, 154, | IC₅₀ > 32 μM |
| 155, 160, 169, 170, 171, 172, 173, 175, 178, 179, 181, 183, 184, 185, | |
| 186, 189, 193, 194 | |

Antagonist Activity of Compounds of Formula (I) on the CXCR4-CXCL12 Signaling Pathway

HEK-293 T cells were transfected to allow measuring the recruitment of Gai2 proteins involved in intracellular signaling by human CXCR4 receptor (hCXCR4) via the BRET technology. Cells were then incubated with various concentrations of examples of the invention before stimulation with an EC₈₀ concentration of CXCL12. However, surprisingly, although many examples of the invention were found to have greater CXCR4-dependent anti-inflammatory activity than IT1t (see Table 1 above), the antagonist activity of several of them was significantly lower than IT1t (see Table 2 below). The reduction of antagonist activity is advantageous as it is expected to avoid undesirable side effects that may be linked to the CXCL12-CXCR4 axis, and thus allows the long-term administration of the respective compounds. Altogether, the results presented here clearly demonstrate that the compounds of the present invention, with reduced antagonist activity, are potent inhibitors of the production of interferons and inflammatory cytokines by specifically targeting CXCR4 while having minimal or no impact on the CXCR4-CXCL12 signaling pathway.

TABLE 2
Antagonist effect of various examples according to the invention
on the CXCR4-CXCL12 signaling pathway. HEK-293 T cells were
co-transfected with several DNA plasmids encoding: hCXCR4; a
G protein (Gαi2); an intracellular effector fused to luciferase
(BRET donor); a plasma membrane effector fused to GFP (BRET
acceptor). Cells were then first incubated for 10 minutes with
different concentrations of various examples of the invention or
IT1t alone before stimulation by an EC₈₀ CXCL12
concentration and luminescence was recorded and IC₅₀ of antagonist
activity were calculated using GraphPad software. The IC₅₀ values of
all the examples presented are the calculations of two results over
2 sets of independent experiments.

| Examples | IC₅₀ |
|---|---|
| IT1t | 0.3 μM |
| 19, 55, 60, 65, 77 | IC₅₀ ≥ 15 μM |

### Example 202: Biological Evaluation

Materials and Methods:

A. Immune Cells Preparation

The blood from healthy donors was obtained from “Etablissement Français du Sang” (convention #19/EFS/029), Paris, France.

Peripheral blood mononuclear cells (PBMCs) were isolated by density centrifugation from peripheral blood leukocyte separation medium, lymphoprep (Stemcell Technologies, Vancouver, Canada).

Monocytes used for in vitro experiments as described in part C/D were purified by positive selection with human CD14 microbeads (Miltenyi Biotec, Bergisch Gladbach, Germany) from PBMCs.

Monocytes were cultured in RPMI 1640 (Sigma-Aldrich, St Louis, USA) containing 10% heat-inactivated Fetal Bovine Serum (FBS, Sigma-Aldrich, St Louis, USA) at 37° C./5% CO₂.

B. Immune Cells Stimulation

PBMCs and monocytes used herein were prepared as described in part A ‘Immune cells preparation’, above. Monocytes as prepared in part C ‘In vitro knockdown using siRNA or CXCR4 antagonist treatment’ and D ‘Quantification of TNFα, IL-6 and IL1β productions’ were incubated with one exemplary compound of formula (I), i.e. Example 77, for 1 hour before stimulation with the TLR7/8 agonist R848 at 1 mg/mL. Monocytes were analysed by flow cytometry. For intracellular TNFα, IL-6 and IL-1p labelling as described in Part D ‘Quantification of TNFα, IL-6 and IL-1β productions’, Brefeldin A (BFA) was added to the cells 30 minutes after R848 stimulation.

C. In Vitro CXCR4 Knockdown Using siRNA or CXCR4 Antagonist Treatment

Isolated monocytes were seeded at 105 cells/100 μL in 96-well plates and incubated at 37° C.

Monocytes were then treated with small interfering RNA (siRNA) which target the chemokine receptor CXCR4 mRNA (siCXCR4) (Smart Pool, Dharmacon, Lafayette, USA), or with control siRNAs (siCTL) and coupled to a transfection agent DOTAP (Roche Applied Science, Penzberg, Germany). The mix was gently mixed and incubated at room temperature for 15 minutes. After incubation, the mix was added to cells in culture at a final concentration of 160 nM. Finally, cells were incubated at 37° C. for 24 hours.

Alternatively, monocytes were treated after seeding with the CXCR4 antagonist AMD3100 (Sigma-Aldrich, USA) at 20 μM for 1 hour.

D. Quantification of TNFα, IL-6 and IL-1β Productions

Monocytes as treated in part C, were washed in phosphate-buffered saline (PBS) and incubated with a viability marker (Zombie Aqua, Biolegend, San Diego, USA) for 30 minutes at room temperature. After washing, cells were resuspended in PBS containing 2% FBS and 2 mM ethylenediaminetetraacetic acid (EDTA) and labelled with anti-CD14 antibody (clone REA599, Miltenyi Biotech, Bergisch Gladbach, Germany), used at 1:100. For intracellular labelling of TNFα, IL-6 and IL-1β, the “Inside Stain” kit (Miltenyi Biotec, Bergisch Gladbach, Germany) was used according to the manufacturer's protocol. Cells were fixed for 20 minutes at room temperature with 250 μL of Inside Fix solution and then labelled in 100 μL of Inside Perm solution containing anti-TNFα antibody (clone cA2), anti-IL-6 antibody (clone REA1037) or anti-IL-1p antibody (clone REA1172) at 1/500 for 30 minutes at room temperature (all from Miltenyi Biotec, Bergisch Gladbach, Germany). Data acquisition was performed on the Canto II flow cytometer using Diva software (BD Biosciences, San Jose, USA). FlowJo software was used to analyse the data.

E. In Vitro CXCR4 Receptor Conformational Changes

G.Validation® assay is a G.CLIPS biotech proprietary fluorescence based assay that assesses conformational changes of a receptor upon activation/inactivation. Recombinant CXCR4 receptor is produced and purified from HEK-293 T cells using G.CLIPS proprietary mixes. Receptor is then reconstituted in detergent buffers containing lipids mimicking the lipidic composition of dendritic cells (DC) and macrophages (SB2L4 and SB3L1). Thereafter, the receptor is labelled with a non-modifying probe allowing the detection of the activation state and conformational change of the receptor upon addition of a ligand. Indeed, the probe emission spectra maximum wavelength (Amax) shifts according to activation/inactivation of the receptor. Thus, kinetic of activation/inactivation of the receptor can be followed by monitoring Amax shift after addition of a ligand over time. Emission spectra were registered for 40 minutes after addition of each ligand.

Activation/inactivation kinetics of 5 μM labelled CXCR4 in SB2L4 and SB3L1 mixes was monitored in presence of the endogenous natural ligand SDF1α (Stromal Cell-Derived Factor-1 alpha, 15 μM, Miltenyi Biotec, Bergisch Gladbach, Germany) and benchmark ligand AMD3100 (CXCR4 antagonist, 100 μM, Sigma-Aldrich, St Louis, USA). Activation/inactivation kinetics were also followed using an exemplary compound of formula (I), i.e. Example 60, as well as irrelevant molecules controls (R2AR ligands, agonist Norepinephrine or inverse agonist ICI 118551) at 150 μM. SDF1α (15 μM), AMD3100 (100 μM) and Example 60 (150 μM) activation effect were also tested on irrelevant G-protein coupled receptor (GPCR) control.

F. In Vivo Evaluation of CXCR4 Antagonistic Activity by Evaluating Mobilisation of Blood Cells

Male C57BL/6 Rj mice (7 weeks old) were obtained from Janvier (Le Genest-Saint-Isle, France). Mice were treated by a single intraperitoneal (i.p.) administration at timepoint 0 h. The experimental groups (n=10) were defined as follows:
Mice of group 1 were treated with vehicle only (0.9% NaCl aqueous solution)
Mice of group 2 were treated with AMD3100 (CXCR4 antagonist, Sigma-Aldrich, St Louis, USA) at 20 mg/kg (volume of 10 mL/kg) in 0.9% NaCl aqueous solution
Mice of group 3 were treated with an exemplary compound of formula (I), i.e. Example 60, at 30 mg/kg (volume of 10 mL/kg) in 0.9% NaCl aqueous solution

| | | | | Route of |
|---|---|---|---|---|
| | Number of | | | adminis- |
| Groups | animals | Treatment | Dose | tration |
| 1 | 10 | Vehicle (0.9% NaCl) | — | i.p. |
| 2 | 10 | AMD3100 in 0.9% NaCl | 20 mg/kg | i.p. |
| 3 | 10 | Example 60 in 0.9% NaCl | 30 mg/kg | i.p. |

After 2:30 h, whole blood (at least 200 μl, max 1 mL) was collected in EDTA tubes before sacrifice by an intracardiac puncture under isoflurane anaesthesia and haematology parameters were determined the same day by laser flow cytometry, optical fluorescence and Laminar Flow Impedance™ using the ProCyte Dx hematology analyser (IDEXX, Eau Claire, USA). Differential leukocyte counts were performed to quantify specific immune cell types. Data are reported as total number of cells (K/μL), mean±S.E.M., as well as the statistical significance versus the vehicle control group using unpaired t-tests.

G. In Vivo Evaluation of Immunomodulating Effect (Decreased Type 1 Interferons (IFN)) in Acute Inflammation Model

Non-fasted male 129S8 mice (12 weeks old, Jackson Laboratory, Bar Harbor, USA) were dosed intranasally (i.n.) once. An exemplary compound of formula (I), i.e. Example 60, was dissolved in PBS at a concentration of 15 mg/mL so that 450 μg in 30 μL would be administered. As a positive control, ibuprofen was also dissolved in PBS at a concentration of 25 mg/mL so that 750 μg in 30 μL would be administered. Vehicle treated animals were dosed i.n. with 30 μL PBS.

After 18 hours, mice were infected i.n. with influenza under isoflurane (5% in O₂) anesthesia. Influenza Avirus (IAV) H3 N2 (X31) was grown in MDCKs (European Collection of Cell Cultures). The virus was obtained from American Type Culture Collection and passaged five times in MDCKs cells before purification. The H3N2 (X31) strain of influenza (30 uL of 800 TCID50) was instilled into each nostril in a drop wise fashion alternating between the two until a volume of 30 μL had been delivered.

| | Number of | | Treatment |
|---|---|---|---|
| Groups | animals | Infection (i.n., 30 μL) | (i.n., 30 μL) |
| 1 | 5 | diluent | vehicle |
| 2 | 7 | IAV H3 N2 (X31-800 TCID50) | vehicle |
| 3 | 7 | IAV H3 N2 (X31-800 TCID50) | Ibuprofen |
| | | | (750 μg) |
| 4 | 7 | IAV H3 N2 (X31-800 TCID50) | Example 60 |
| | | | (450 μg) |

Three days after influenza or sham challenge, mice from each group were culled with pentobarbitone overdose (i.p.). The trachea was then isolated by a midline incision in the neck and separation of the muscle layers. A small incision was made into the trachea and a plastic cannula was inserted and secured in place with a suture. The airways were then lavaged by flushing out the lungs using 0.5 mL of PBS. This procedure was repeated until the recovered volume was 1.6 mL. The isolated bronchoalveolar lavage fluid (BALF) was then centrifuged at 1500 rpm for 10 minutes at 4° C. and the supernatant was aliquoted (400 μL) at −80° C. BALF supernatant was evaluated for IFNα (eBioscience, Frankfurt am Main, Germany), IFNβ (Biolegend, San Diego, USA) and IFNλ2/3 (R&D Systems, Minneapolis, USA) concentrations using ELISA kits as per the manufacturer's instructions. Optical density was measured at 450 nM using a microplate reader (SpectraMax 340PC, Molecular Devices, San Jose, USA). Concentrations of IFN were determined using SoftMax Pro v. 6.4 (Molecular Devices, San Jose, USA). Data are reported as IFN (pg/mL), mean±S.E.M. (standard error of the mean) as well as the statistical significance versus influenza infected vehicle treated group using t-tests.

H. In Vivo Pristane-Induced Lupus Mouse Model

Female Balb/c mice (6 to 8 weeks, ENVIGO, Indianapolis, USA) received a single i.p. injection with 0.5 mL of pristane (Sigma-Aldrich, St Louis, USA) to induce a lupus-like disease. That same day, once daily treatment was started (10 mL/kg), either with vehicle control (PBS) i.p., with prednisolone (Sigma-Aldrich, St Louis, USA) in PBS p.o. at a dose of 15 mg/kg (positive control), or with an exemplary compound of formula (I), i.e. Example 60, at a dose of either 3 mg/kg, 10 mg/kg or 30 mg/kg in PBS i.p. After 4, 6 and 8 weeks of dosing, sample bleeds for each mouse were collected via the orbital sinus, and immediately cold processed to sera and stored (50 μl) at −80° C. until analysis. In serum, anti-dsDNA antibody titers are determined using ELISA. Optical density was measured at 450 nM (OD450) using a microplate reader. Data are reported as anti-dsDNA Ab titers (OD), mean±S.E.M. as well as the statistical significance versus pristane-induced vehicle treated group using t-tests.

| | Number of | |
|---|---|---|
| Groups | animals | Treatment once daily |
| 1 | 8 | Vehicle (PBS), i.p. |
| 2 | 8 | Prednisolone (PBS), 15 mg/kg, p.o. |
| 3 | 8 | Example 60 (PBS), 3 mg/kg, i.p. |
| 4 | 8 | Example 60 (PBS), 10 mg/kg, i.p. |
| 5 | 8 | Example 60 (PBS), 30 mg/kg, i.p. |

Results:

Role of CXCR4 in the Effect on TNF-α, IL-6 and IL-1β Productions by Monocytes from Healthy Donors

The anti-inflammatory effect of Example 77 on monocytes from a healthy donor was evaluated in a context where CXCR4 expression on cell membrane was suppressed or blocked. The CXCR4 gene was silenced using small interfering RNA (siRNA) prior to incubation with Example 77 (50 nM) and R848 (1 μg/mL). CXCR4 siRNA restored TNFα, IL-6 and IL-1β productions by activated monocytes in the presence of Example 77 (see FIGS. 1 and 2). Alternatively, CXCR4 was blocked using AMD3100, a CXCR4 antagonist (20 μM), and also restored TNFα and IL-1β productions by activated monocytes in the presence of Example 77 (10, 50 and 500 nM) (see FIGS. 3 to 5). These results unambiguously demonstrate that CXCR4 is required for the inhibitory activity of the compounds of the invention, including Example 77.

In Vitro Effect on CXCR4 Receptor Conformational Changes

First the effect of benchmark ligands SDF1α and AMD3100 on CXCR4 structural activation/inactivation was monitored in SB2L4 and SB3L1. After registration of basal state activation emission spectra at TO, SDF1α and emission spectra were registered for 40 min, and then AMD3100 was added and emission spectra were recorded for another 40 min. Addition of SDF1α led to structural activation of CXCR4 as shown by the red shift in Amax, whereas addition of AMD3100 reversed SDF1α effect with a blue shift in Amax (see FIG. 6A).

Example 60 effect on CXCR4 was tested and led to blue shift in Amax over basal state, showing on target effect of the compound leading to a decrease in basal state activation of the CXCR4 receptor. Addition of AMD3100 above Example 60 did not reverse Example 60 effect, suggesting that the two molecules might have different binding sites (see FIG. 6A).

β2AR ligands, agonist Norepinephrine or inverse agonist ICI 118 551, were also tested on CXCR4 as controls and did not show any significant effect on the receptor activation state. An irrelevant GPCR was also used as control. Neither benchmark ligands SDF1α and AMD3100, nor Example 60 showed any effect on the irrelevant GPCR activation state (see FIG. 6B).

These results show that the compounds of the invention, including Example 60, induce confirmational changes of the CXCR4 receptor, thereby confirming on-target activity.

In Vivo Evaluation of Absence of CXCR4 Antagonistic Activity by Evaluating Mobilisation of Blood Cells

Compared to the vehicle negative control group (single i.p. injection of 0.9% NaCl), significantly higher numbers of white blood cells were found in the blood 2:30 h after a single i.p. injection of the CXCR4 antagonist AMD3100 at a dose of 20 mg/kg. In contrast, the numbers of white blood cells found in the blood after a single i.p. treatment with Example 60 at a dose of 30 mg/kg, were not increased compared to those in the vehicle control group (see FIG. 7A). This effect is shown in particular for neutrophils, monocytes, lymphocytes and eosinophils (see FIGS. 7B to 7E). These data clearly confirm the in vitro BRET assay results demonstrating the reduction in CXCR4 antagonistic activity in the compounds of the invention, including Example 60, thereby avoiding undesirable side effects linked to the CXCL12-CXCR4 axis and thus allowing long-term administration of the compounds.

In Vivo Evaluation of Immunomodulating Effect (Decreased Type 1 IFNs) in Acute Inflammation Model

Male 129S8 mice show significantly increased levels of type 1 IFNs in the BALF 3 days after infection with influenza strain H3 N2 (X31). By a single i.n. administration of ibuprofen, a known anti-inflammatory agent, significantly lower concentrations of IFNα, IFNβ and IFNλ2/3 are detected in the BALF (see FIGS. 8A to 8C). Furthermore, when administering Example 60 once i.n. at a dose of 450 μg, the observed concentrations of all type 1 IFNs are significantly decreased as well, but even lower than the concentrations observed after ibuprofen treatment (see FIGS. 8A to 8C).

These data demonstrate the anti-inflammatory effect of the compounds of the invention in vivo, including Example 60, in an acute influenza model.

In Vivo Evaluation of Effect on Anti-Ds DNA Ab Titers in Pristane-Induced Lupus Mouse Model

In order to evaluate the effect of Example 60 in the pristane-induced lupus mouse model, sera were analyzed for anti-dsDNA antibody titers at 4, 6 and 8 weeks, after simultaneous induction of disease using pristane and the start of daily treatment. Increased anti-dsDNA Ab titers are one of the features of lupus in humans as well, and therefore considered a relevant endpoint to evaluate treatment effect. As a positive control, prednisolone (15 mg/kg, p.o.) was used. Vehicle-treated pristane-induced mice showed high mean anti-dsDNA Ab titers, whereas treatment with prednisolone significantly reduced anti-dsDNA Ab titers as of week 4. At week 4, all doses of Example 60 treatment showed reduced anti-dsDNA Ab titers. At week 6, significantly lower titers were measured for the 30 mg/kg dose group. After 8 weeks, anti-dsDNA Ab titers in both the 30 mg/kg and the 10 mg/kg treatment groups were significantly decreased and in the same range as seen for prednisolone (see FIG. 9).

These data confirm the in vitro anti-inflammatory effects of the compounds of the invention, including Example 60, and the impact of these effects in an in vivo lupus mouse model.

1. A compound of formula (I) 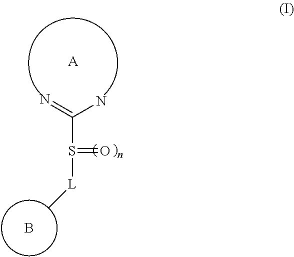
wherein:
ring A is any one of the following groups A1 to A11: 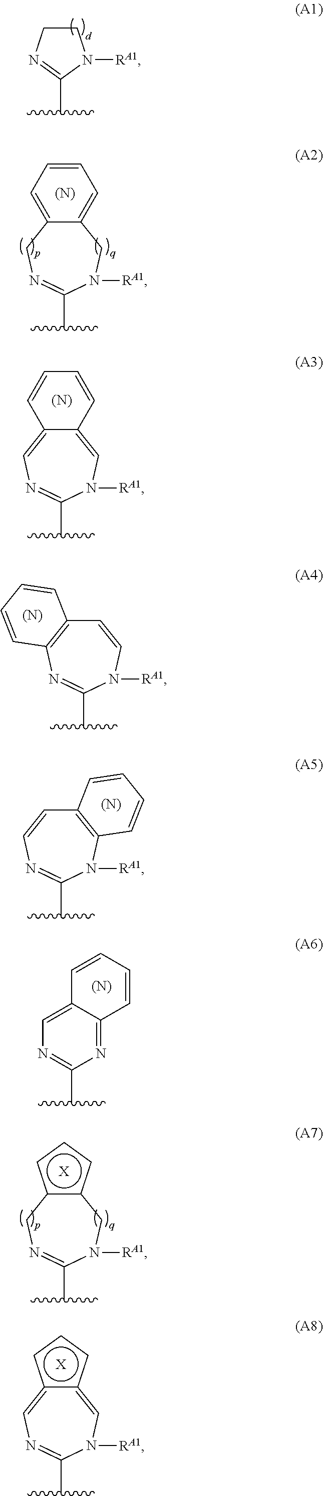 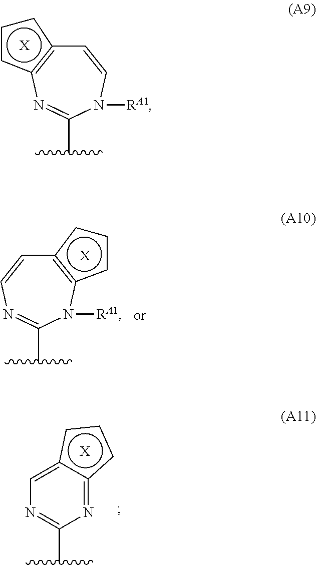
wherein d is 1, 2 or 3;
wherein p is 0, 1, 2 or 3, and q is 0, 1 or 2, with the proviso that p and q are not both 0;
wherein the symbol “(N)” depicted inside a ring indicates that 0, 1, 2 or 3 ring atom(s) of the respective ring is/are nitrogen ring atom(s);
wherein the symbol “X” depicted inside a 5-membered ring indicates that the corresponding ring is aromatic and that 1, 2 or 3 ring atom(s) of said ring is/are each independently selected from nitrogen, oxygen and sulfur, while the remaining ring atoms are carbon atoms; and
wherein ring A is optionally substituted with one or more groups R^{A2};
n is 0, 1 or 2;
L is a covalent bond or C₁₋₅ alkylene, wherein said alkylene is optionally substituted with one or more groups R^{L}, wherein one or more —CH₂— units comprised in said alkylene are each optionally replaced by a group independently selected from —O—, —NH—, —N(C₁₋₅ alkyl)-, —CO—, —S—, —SO—, —SO₂—, carbocyclylene, and heterocyclylene, and wherein each R^{L} is independently selected from —OH, —O(C₁₋₅ alkyl), —SH, —S(C₁₋₅ alkyl), —NH₂, —NH(C₁₋₅ alkyl), —N(C₁₋₅ alkyl)(C₁₋₅ alkyl), halogen, —CF₃, —CN, C₁₋₅ alkyl, cycloalkyl, and heterocycloalkyl;
if ring A is a group A1, then ring B is selected from any one of the following groups: 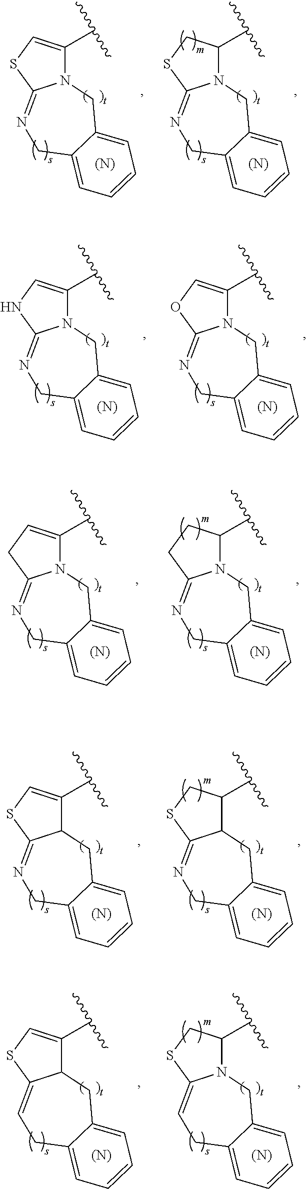 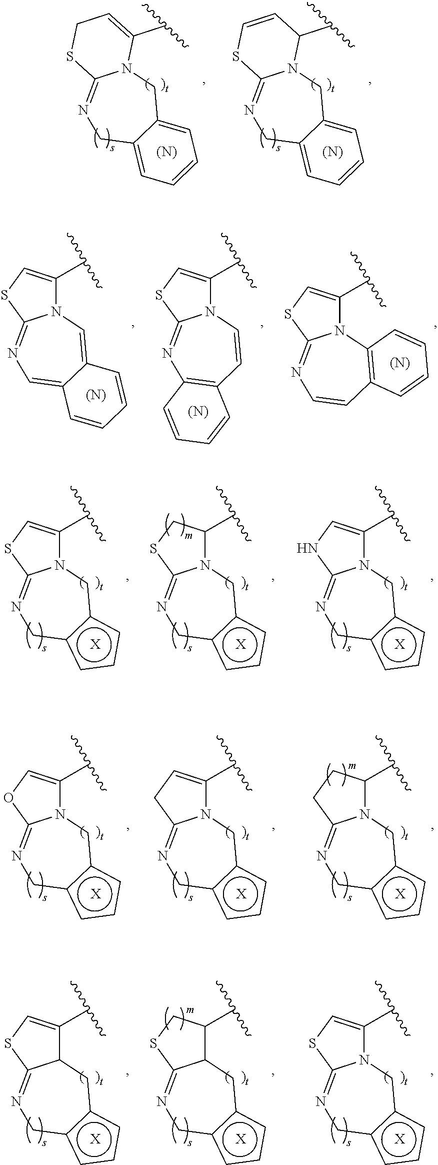 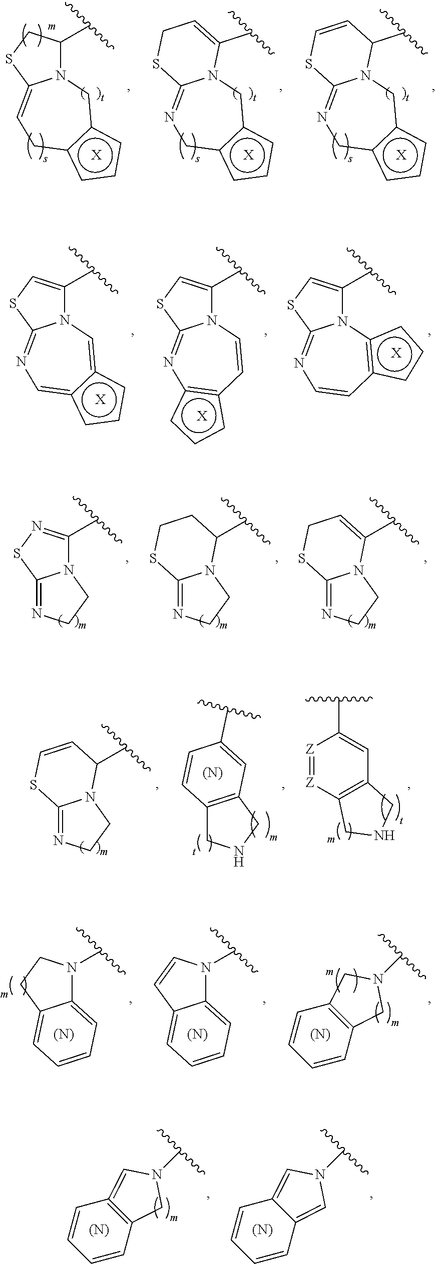 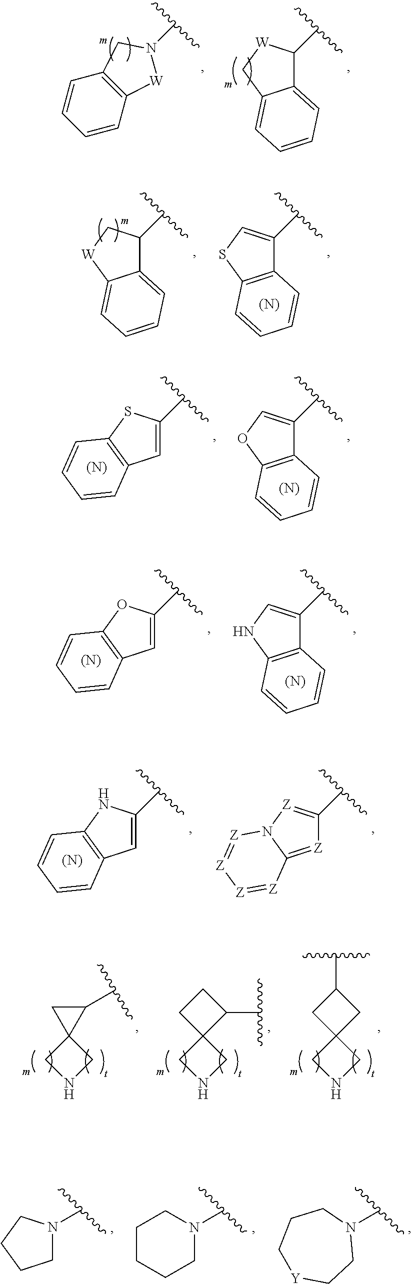 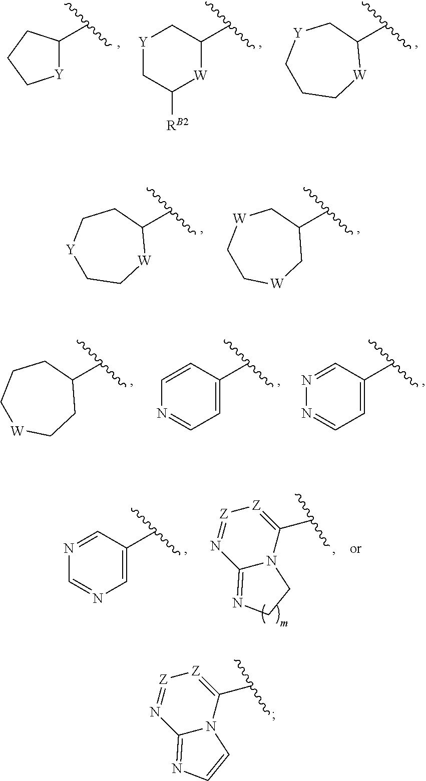
wherein each of the above-depicted groups is optionally substituted with one or more groups R^{B1};
wherein each s is independently 0, 1 or 2;
wherein each t is independently 0, 1, 2 or 3;
wherein each m is independently 1, 2 or 3;
wherein each ring atom W is independently selected from S, O, SO₂ and NH;
wherein each ring atom Y is independently selected from S, O, SO₂, NH and CH₂;
wherein each ring atom Z is independently C or N;
wherein the symbol “(N)” depicted inside a ring indicates that 0, 1, 2 or 3 ring atom(s) of the respective ring is/are nitrogen ring atom(s);
wherein the symbol “N” depicted inside a ring indicates that 1, 2 or 3 ring atom(s) of the respective ring is/are nitrogen ring atom(s); and
wherein the symbol “X” depicted inside a 5-membered ring indicates that the corresponding ring is aromatic and that 1, 2 or 3 ring atom(s) of said ring is/are each independently selected from nitrogen, oxygen and sulfur, while the remaining ring atoms are carbon atoms;
if ring A is a group A2, A3, A4, A5, A7, A8, A9 or A10, then ring B is selected from any one of the following groups: 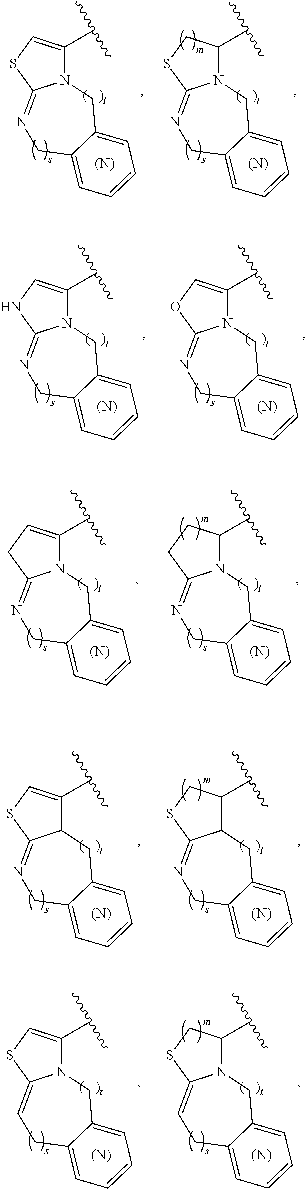 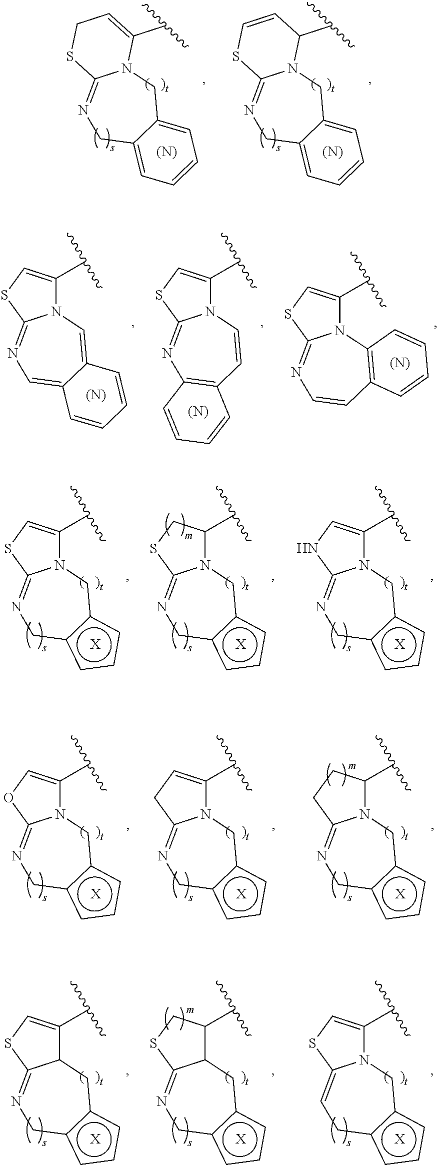 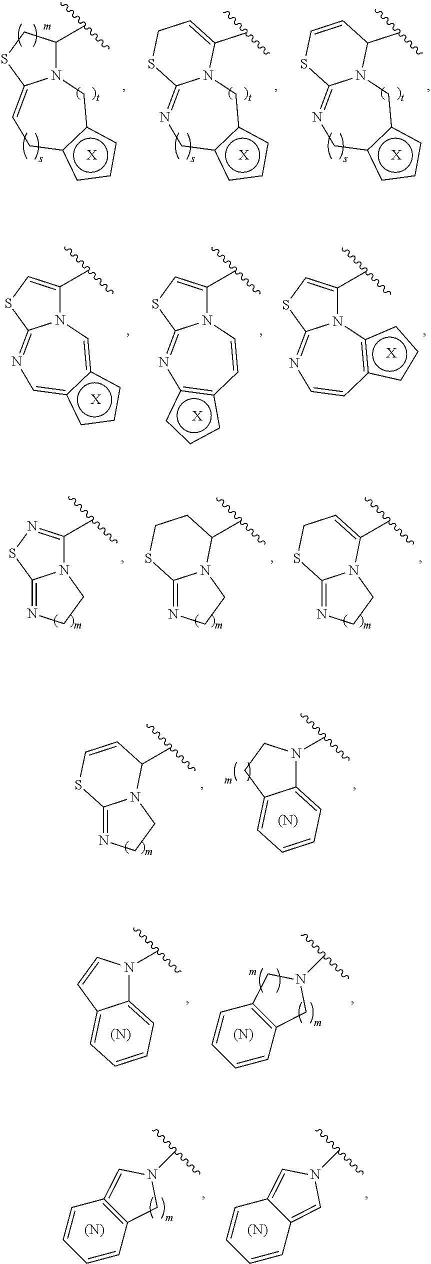 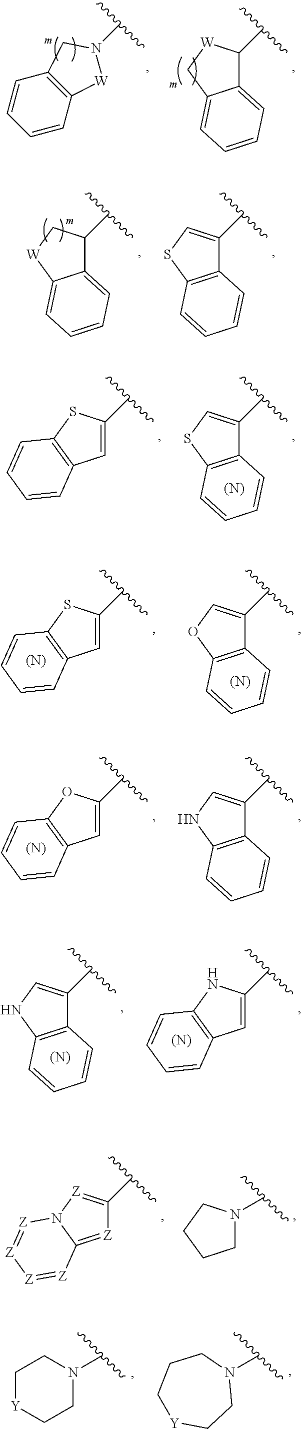 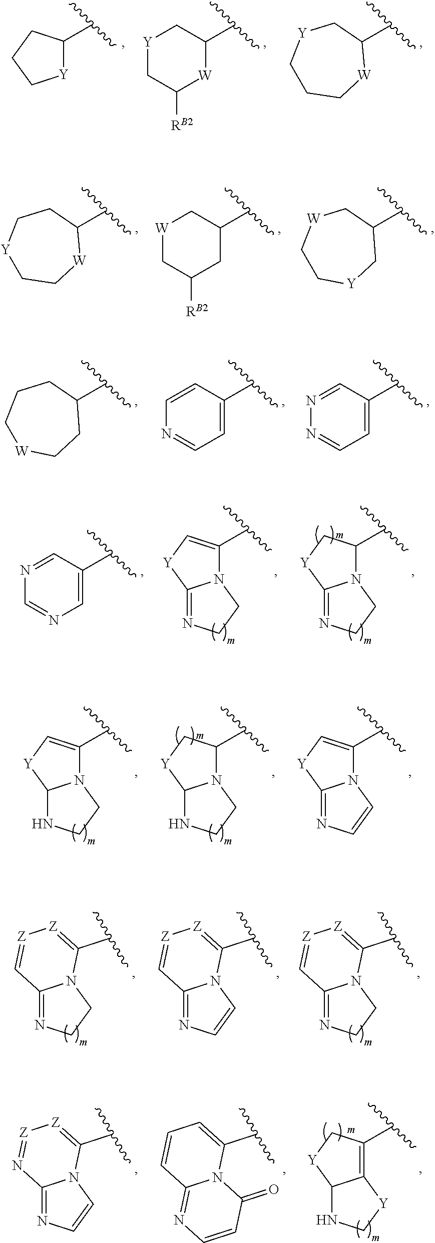 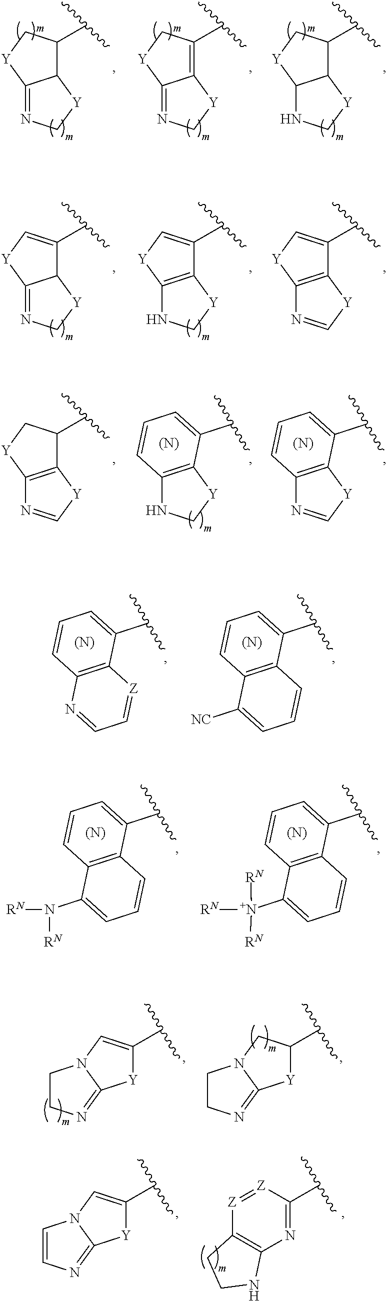 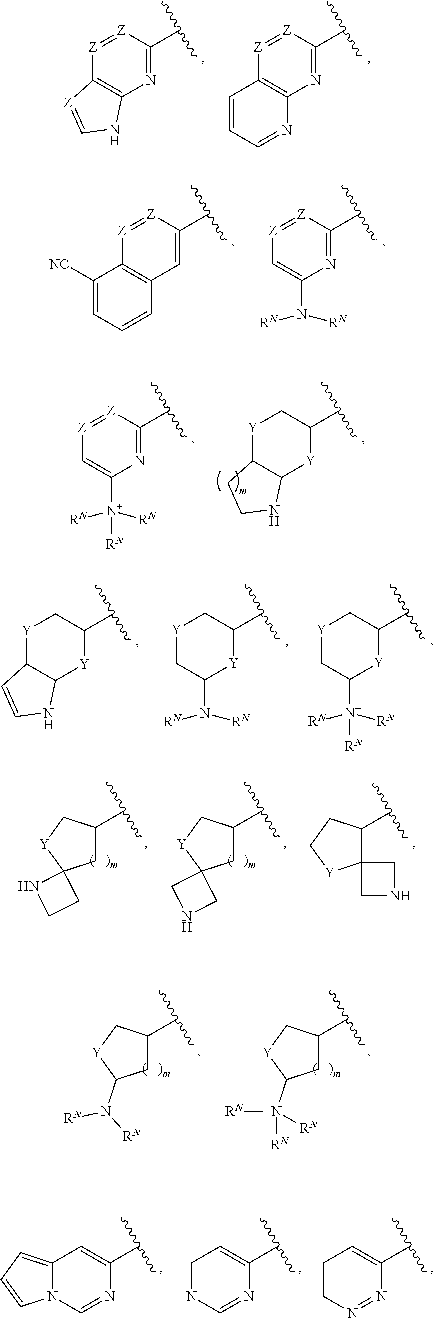 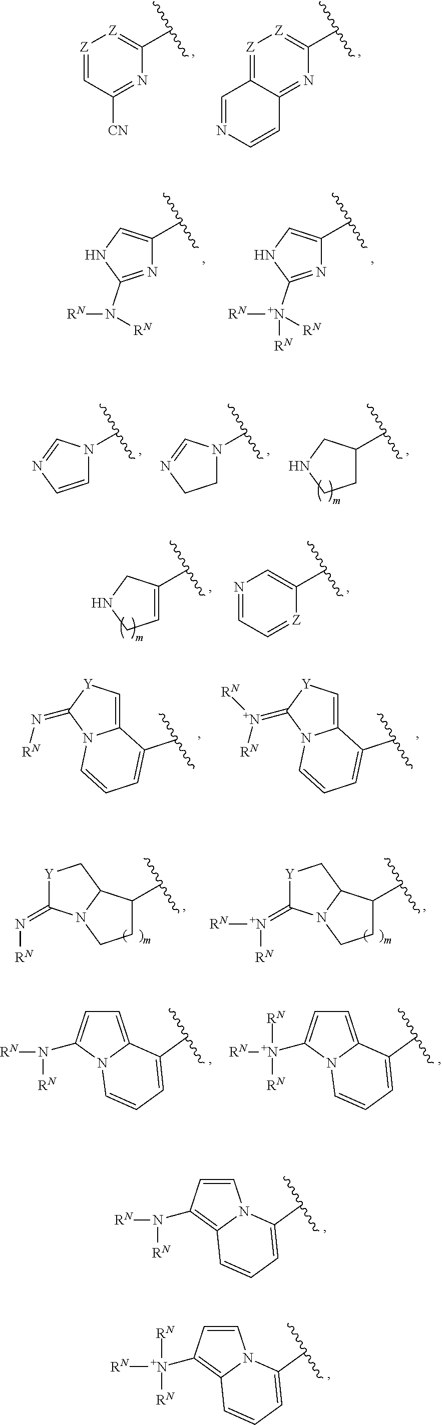 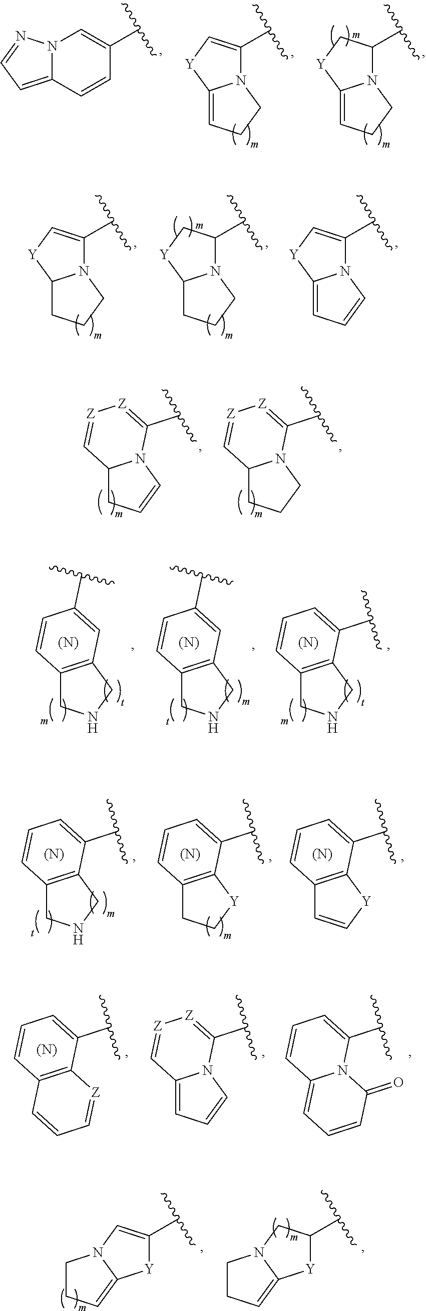 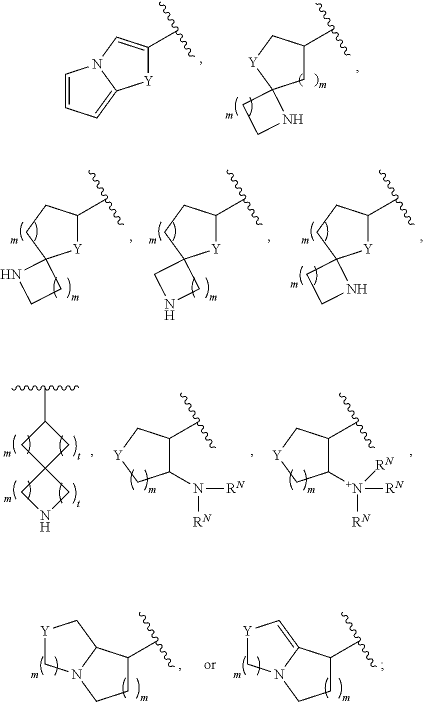
wherein each of the above-depicted groups is optionally substituted with one or more groups R^{B1};
wherein each s is independently 0, 1 or 2;
wherein each t is independently 0, 1, 2 or 3;
wherein each m is independently 1, 2 or 3;
wherein each ring atom W is independently selected from S, O, SO₂ and NH;
wherein each ring atom Y is independently selected from S, O, SO₂, NH and CH₂;
wherein each ring atom Z is independently C or N;
wherein the symbol “(N)” depicted inside a ring indicates that 0, 1, 2 or 3 ring atom(s) of the respective ring is/are nitrogen ring atom(s);
wherein the symbol “N” depicted inside a ring indicates that 1, 2 or 3 ring atom(s) of the respective ring is/are nitrogen ring atom(s); and
wherein the symbol “X” depicted inside a 5-membered ring indicates that the corresponding ring is aromatic and that 1, 2 or 3 ring atom(s) of said ring is/are each independently selected from nitrogen, oxygen and sulfur, while the remaining ring atoms are carbon atoms;
if ring A is a group A6 or A11, then ring B is selected from any one of the following groups: 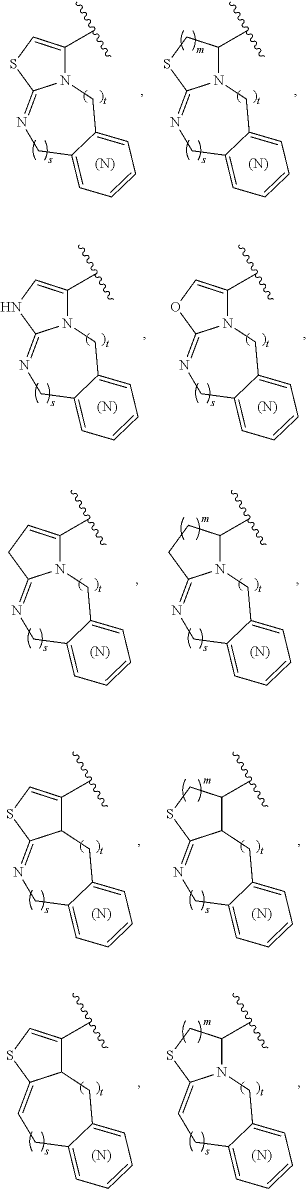 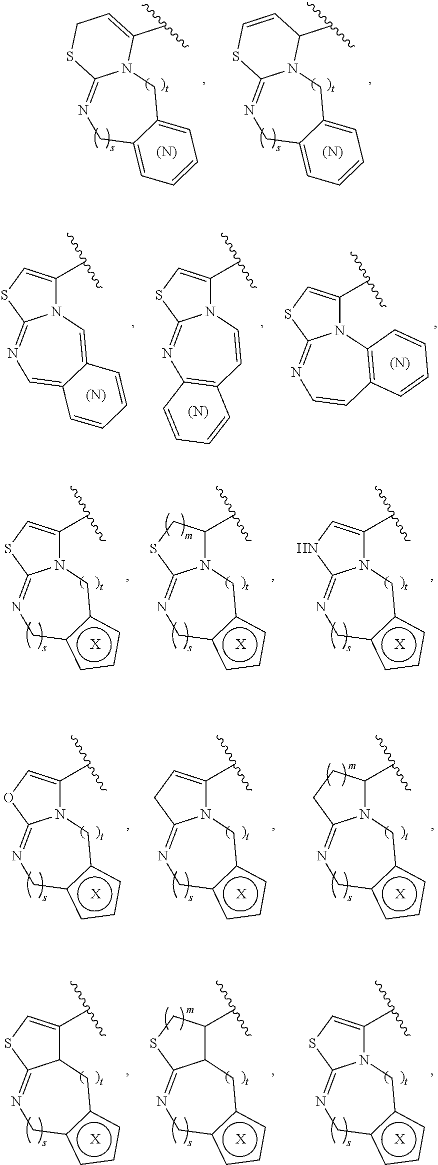 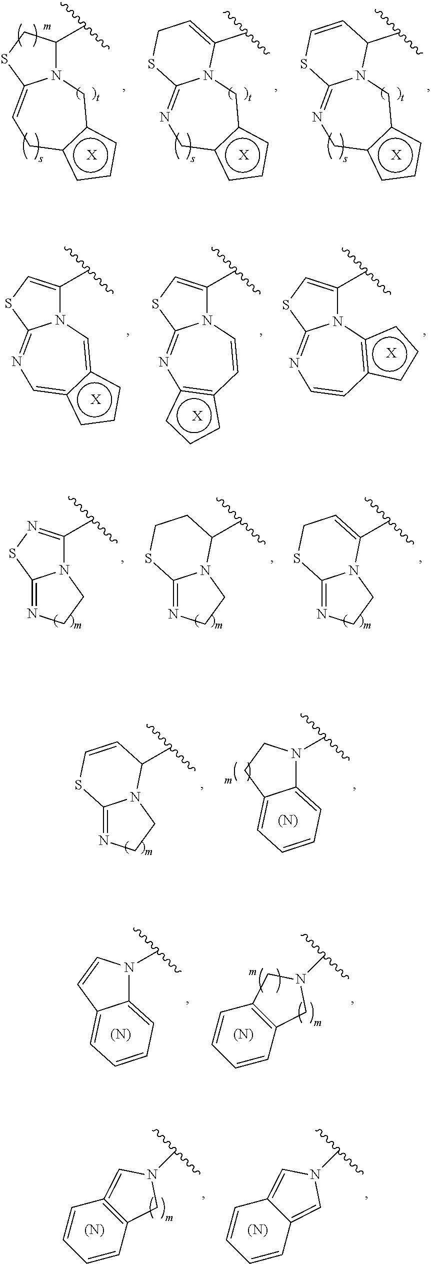 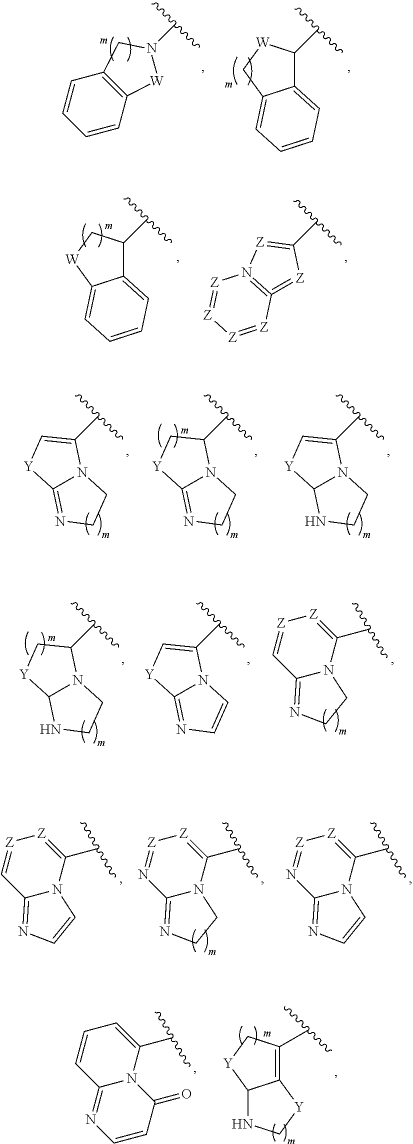 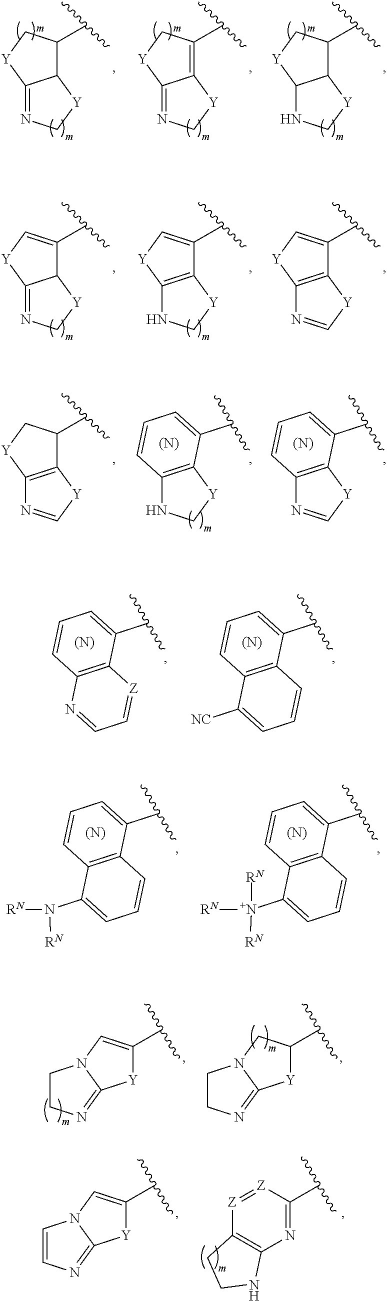 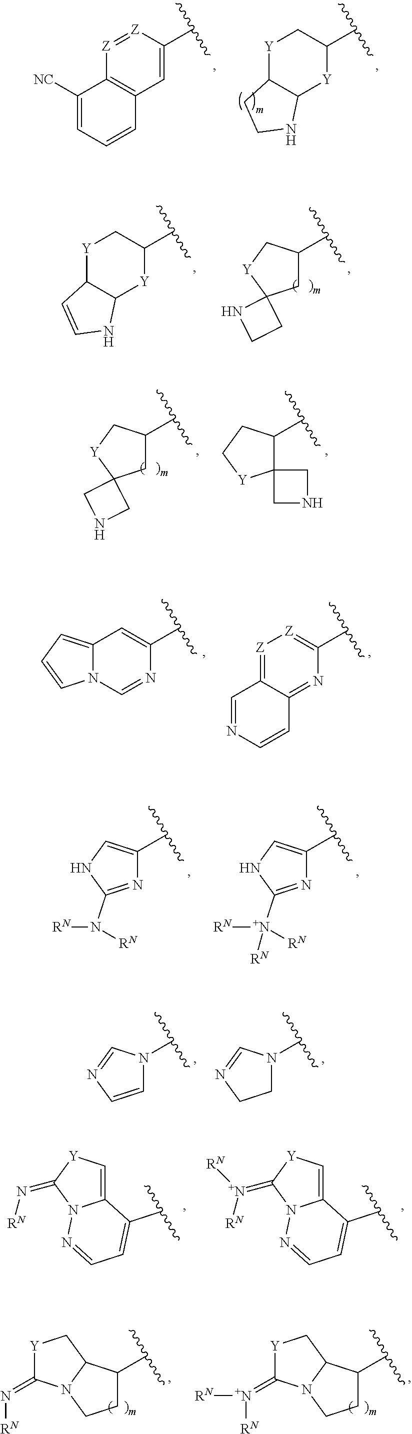 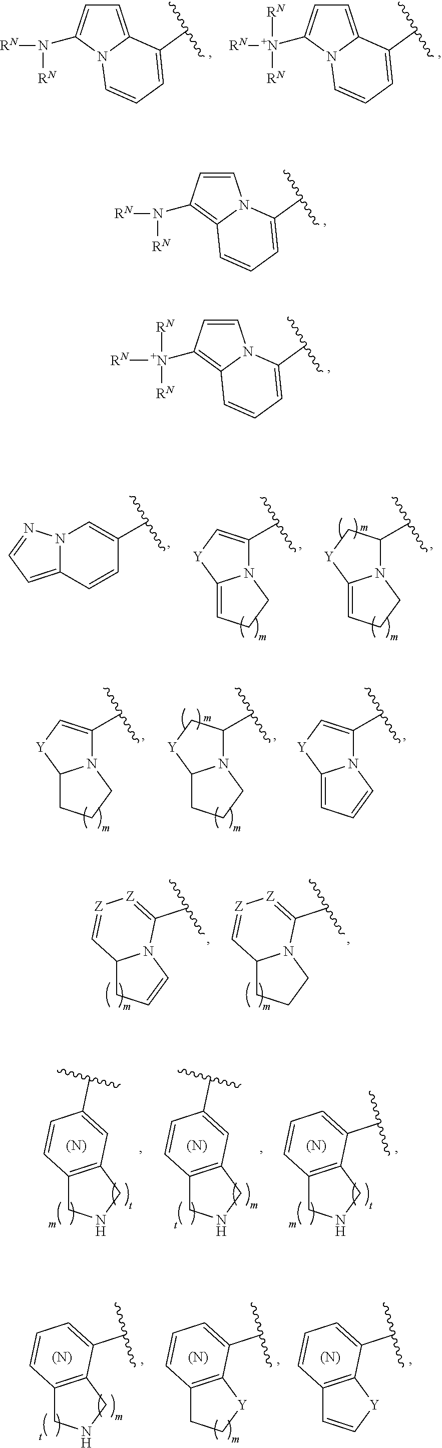 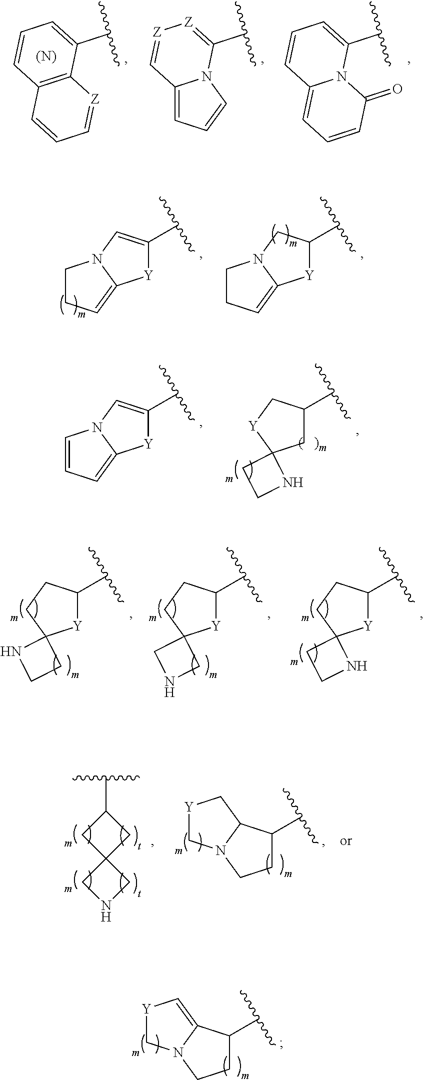
wherein each of the above-depicted groups is optionally substituted with one or more groups R^{B1};
wherein each s is independently 0, 1 or 2;
wherein each t is independently 0, 1, 2 or 3;
wherein each m is independently 1, 2 or 3;
wherein each ring atom W is independently selected from S, O, SO₂ and NH;
wherein each ring atom Y is independently selected from S, O, SO₂, NH and CH₂;
wherein each ring atom Z is independently C or N;
wherein the symbol “(N)” depicted inside a ring indicates that 0, 1, 2 or 3 ring atom(s) of the respective ring is/are nitrogen ring atom(s); and
wherein the symbol “X” depicted inside a 5-membered ring indicates that the corresponding ring is aromatic and that 1, 2 or 3 ring atom(s) of said ring is/are each independently selected from nitrogen, oxygen and sulfur, while the remaining ring atoms are carbon atoms;
R^{A1} is selected from hydrogen, C₁₋₅ alkyl, C₂₋₅ alkenyl, C₂₋₅ alkynyl, —CO(C₁₋₅ alkyl), —COO(C₁₋₅ alkyl), carbocyclyl, and heterocyclyl, wherein said alkyl, said alkenyl, said alkynyl, the alkyl moiety in said —CO(C₁₋₅ alkyl), and the alkyl moiety in said —COO(C₁₋₅ alkyl) are each optionally substituted with one or more groups R^{Alk}, and further wherein said carbocyclyl and said heterocyclyl are each optionally substituted with one or more groups R^{Cyc};
each R^{A2} is independently selected from C₁₋₅ alkyl, C₂₋₅ alkenyl, C₂₋₅ alkynyl, —(C₀₋₅ alkylene)-R^{A21}, —(C₂₋₅ alkenylene)-R^{A21}, and —(C₂₋₅ alkynylene)-R^{A21}, wherein said alkyl, said alkenyl, said alkynyl, said alkylene, said alkenylene, and said alkynylene are each optionally substituted with one or more groups R^{Alk}, and further wherein one or more —CH₂— units comprised in said alkylene, said alkenylene, or said alkynylene are each optionally replaced by a group independently selected from —O—, —NH—, —N(C₁₋₅ alkyl)-, —CO—, —S—, —SO—, and —SO₂—;
wherein any two groups R^{A2}, which are attached to the same ring atom of ring A, may also be mutually joined to form, together with the ring atom that they are attached to, a cycloalkyl or a heterocycloalkyl, wherein said cycloalkyl or said heterocycloalkyl is optionally substituted with one or more groups R^{Cyc};
wherein any two groups R^{A2}, which are attached to distinct ring atoms of ring A, may also be mutually joined to form a C₁₋₅ alkylene which is optionally substituted with one or more groups R^{Cyc}, and wherein one or more —CH₂— units comprised in said alkylene are each optionally replaced by a group independently selected from —O—, —NH—, —N(C₁₋₅ alkyl)-, —CO—, —S—, —SO—, —SO₂—, and phen-1,2-diyl, wherein said phen-1,2-diyl is optionally substituted with one or more groups R^{Cyc}; and
wherein any one group R^{A2} may also be mutually joined with R^{A1} to form a C₁₋₅ alkylene which is optionally substituted with one or more groups R^{Cyc}, and wherein one or more —CH₂— units comprised in said alkylene are each optionally replaced by a group independently selected from —O—, —NH—, —N(C₁₋₅ alkyl)-, —CO—, —S—, —SO—, —SO₂—, and phen-1,2-diyl, wherein said phen-1,2-diyl is optionally substituted with one or more groups R^{Cyc};
each R^{A21} is independently selected from halogen, C₁₋₅ haloalkyl, —O(C₁₋₅ haloalkyl), —CN, —OR^{A22}, —NR^{A22}R^{A22}, —NR^{A22}OR^{A22}, —COR^{A22}, —COOR^{A22}, —OCOR^{A22}, —CONR^{A22}R^{A22}, —NR^{A22}COR^{A22}, —NR^{A22}COOR^{A22}, —OCONR^{A22}R^{A22}, —SR^{A22}, —SOR^{A22}, —SO₂R^{A22}, —SO₂NR^{A22}R^{A22}, —NR^{A22}SO₂R^{A22}, —SO₃R^{A22}, —NO₂, carbocyclyl, and heterocyclyl, wherein said carbocyclyl and said heterocyclyl are each optionally substituted with one or more groups R^{Cyc};
each R^{A22} is independently selected from hydrogen, C₁₋₅ alkyl, C₂₋₅ alkenyl, C₂₋₅ alkynyl, carbocyclyl, and heterocyclyl, wherein said alkyl, said alkenyl and said alkynyl are each optionally substituted with one or more groups R^{Alk}, and further wherein said carbocyclyl and said heterocyclyl are each optionally substituted with one or more groups R^{Cyc};
each R^{N} is independently selected from hydrogen, C₁₋₅ alkyl, C₂₋₅ alkenyl, C₂₋₅ alkynyl, —O(C₁₋₅ alkyl), —CO(C₁₋₅ alkyl), —COO(C₁₋₅ alkyl), carbocyclyl, and heterocyclyl, wherein said alkyl, said alkenyl, said alkynyl, the alkyl moiety in said —O(C₁₋₅ alkyl), the alkyl moiety in said —CO(C₁₋₅ alkyl), and the alkyl moiety in said —COO(C₁₋₅ alkyl) are each optionally substituted with one or more groups R^{Alk}, wherein said carbocyclyl and said heterocyclyl are each optionally substituted with one or more groups R^{Cyc}, and further wherein any two groups R^{N} which are attached to the same nitrogen atom may also be mutually joined to form, together with the nitrogen atom that they are attached to, a heterocyclyl which is optionally substituted with one or more groups R^{Cyc};
each R^{B1} is independently selected from C₁₋₅ alkyl, C₂₋₅ alkenyl, C₂₋₅ alkynyl, —(C₀₋₅ alkylene)-R^{B11}, —(C₂₋₅ alkenylene)-R^{B11}, —(C₂₋₅ alkynylene)-R^{B11}, and ═R^{B13}, wherein said alkyl, said alkenyl, said alkynyl, said alkylene, said alkenylene, and said alkynylene are each optionally substituted with one or more groups R^{Alk}, and further wherein one or more —CH₂— units comprised in said alkylene, said alkenylene, or said alkynylene are each optionally replaced by a group independently selected from —O—, —NH—, —N(C₁₋₅ alkyl)-, —CO—, —S—, —SO—, and —SO₂—;
wherein any two groups R^{B1}, which are attached to the same ring atom of ring B, may also be mutually joined to form, together with the ring atom that they are attached to, a cycloalkyl or a heterocycloalkyl, wherein said cycloalkyl or said heterocycloalkyl is optionally substituted with one or more groups R^{Cyc}; and
wherein any two groups R^{B1}, which are attached to distinct ring atoms of ring B, may also be mutually joined to form a C₁₋₅ alkylene which is optionally substituted with one or more groups R^{Cyc}, and wherein one or more —CH₂— units comprised in said alkylene are each optionally replaced by a group independently selected from —O—, —NH—, —N(C₁₋₅ alkyl)-, —CO—, —S—, —SO—, and —SO₂—;
each R^{B11} is independently selected from halogen, C₁₋₅ haloalkyl, —O(C₁₋₅ haloalkyl), —CN, —OR^{B12}, —NR^{B12}R^{B12}, —N⁺R^{B12}R^{B12}R^{B12}, —NR^{B12}OR^{B12}, —COR^{B12}, —COOR^{B12}, —OCOR^{B12}, —CONR^{B12}R^{B12}, —NR^{B12}COR^{B12}, —NR^{B12}COOR^{B12}, —OCONR^{B12}R^{B12}, —SR^{B12}, —SOR^{B12}, —SO₂R^{B12}, —SO₂NR^{B12}R^{B12}, —NR^{B12}SO₂R^{B12}, —SO₃R^{B12}, —NO₂, carbocyclyl, and heterocyclyl, wherein said carbocyclyl and said heterocyclyl are each optionally substituted with one or more groups R^{Cyc};
each R^{B12} is independently selected from hydrogen, C₁₋₅ alkyl, C₂₋₅ alkenyl, C₂₋₅ alkynyl, carbocyclyl, and heterocyclyl, wherein said alkyl, said alkenyl and said alkynyl are each optionally substituted with one or more groups R^{Alk}, and further wherein said carbocyclyl and said heterocyclyl are each optionally substituted with one or more groups R^{Cyc};
each R^{B13} is independently selected from ═O, ═S, and ═N—R^{B12};
each R^{B2} is independently selected from C₁₋₅ alkyl, C₂₋₅ alkenyl, C₂₋₅ alkynyl, —(C₀₋₅ alkylene)-R^{B21}, —(C₂₋₅ alkenylene)-R^{B21}, and —(C₂₋₅ alkynylene)-R^{B21}, wherein said alkyl, said alkenyl, said alkynyl, said alkylene, said alkenylene, and said alkynylene are each optionally substituted with one or more groups R^{Alk}, and further wherein one or more —CH₂— units comprised in said alkylene, said alkenylene, or said alkynylene are each optionally replaced by a group independently selected from —O—, —NH—, —N(C₁₋₅ alkyl)-, —CO—, —S—, —SO—, and —SO₂—;
each R^{B21} is independently selected from halogen, C₁₋₅ haloalkyl, —O(C₁₋₅ haloalkyl), —CN, —OR^{B12}, —COR^{B12}, —COOR^{B12}, —OCOR^{B12}, —CONR^{B12}R^{B12}, —OCONR^{B12}R^{B12}, —SR^{B12}, —SOR^{B12}, —SO₂R^{B12}, —SO₂NR^{B12}R^{B12}, —NR^{B12}SO₂R^{B12}, —SO₃R^{B12}, —NO₂, carbocyclyl, and heterocyclyl, wherein said carbocyclyl and said heterocyclyl are each optionally substituted with one or more groups R^{Cyc};
each R^{Alk} is independently selected from —OH, —O(C₁₋₅ alkyl), —O(C₁₋₅ alkylene)-OH, —O(C₁₋₅ alkylene)-O(C₁₋₅ alkyl), —SH, —S(C₁₋₅ alkyl), —S(C₁₋₅ alkylene)-SH, —S(C₁₋₅ alkylene)-S(C₁₋₅ alkyl), —NH₂, —NH(C₁₋₅ alkyl), —N(C₁₋₅ alkyl)(C₁₋₅ alkyl), —NH—OH, —N(C₁₋₅ alkyl)-OH, —NH—O(C₁₋₅ alkyl), —N(C₁₋₅ alkyl)-O(C₁₋₅ alkyl), halogen, C₁₋₅ haloalkyl, —O(C₁₋₅ haloalkyl), —CN, —NO₂, —CHO, —CO(C₁₋₅ alkyl), —COOH, —COO(C₁₋₅ alkyl), —O—CO(C₁₋₅ alkyl), —CO—NH₂, —CO—NH(C₁₋₅ alkyl), —CO—N(C₁₋₅ alkyl)(C₁₋₅ alkyl), —NH—CO(C₁₋₅ alkyl), —N(C₁₋₅ alkyl)-CO(C₁₋₅ alkyl), —NH—COO(C₁₋₅ alkyl), —N(C₁₋₅ alkyl)-COO(C₁₋₅ alkyl), —O—CO—NH(C₁₋₅ alkyl), —O—CO—N(C₁₋₅ alkyl)(C₁₋₅ alkyl), —SO₂—NH₂, —SO₂—NH(C₁₋₅ alkyl), —SO₂—N(C₁₋₅ alkyl)(C₁₋₅ alkyl), —NH—SO₂—(C₁₋₅ alkyl), —N(C₁₋₅ alkyl)-SO₂—(C₁₋₅ alkyl), —SO₂—(C₁₋₅ alkyl), —SO—(C₁₋₅ alkyl), aryl, heteroaryl, cycloalkyl, heterocycloalkyl, and -L^{X}-R^{X}, wherein said aryl, said heteroaryl, said cycloalkyl, and said heterocycloalkyl are each optionally substituted with one or more groups independently selected from C₁₋₅ alkyl, C₂₋₅ alkenyl, C₂₋₅ alkynyl, halogen, C₁₋₅ haloalkyl, —O(C₁₋₅ haloalkyl), —CN, —OH, —O(C₁₋₅ alkyl), —SH, —S(C₁₋₅ alkyl), —NH₂, —NH(C₁₋₅ alkyl), and —N(C₁₋₅ alkyl)(C₁₋₅ alkyl);
each R^{Cyc} is independently selected from C₁₋₅ alkyl, C₂₋₅ alkenyl, C₂₋₅ alkynyl, —OH, —O(C₁₋₅ alkyl), —O(C₁₋₅ alkylene)-OH, —O(C₁₋₅ alkylene)-O(C₁₋₅ alkyl), —SH, —S(C₁₋₅ alkyl), —S(C₁₋₅ alkylene)-SH, —S(C₁₋₅ alkylene)-S(C₁₋₅ alkyl), —NH₂, —NH(C₁₋₅ alkyl), —N(C₁₋₅ alkyl)(C₁₋₅ alkyl), —NH—OH, —N(C₁₋₅ alkyl)-OH, —NH—O(C₁₋₅ alkyl), —N(C₁₋₅ alkyl)-O(C₁₋₅ alkyl), halogen, C₁₋₅ haloalkyl, —O(C₁₋₅ haloalkyl), —CN, —NO₂, —CHO, —CO(C₁₋₅ alkyl), —COOH, —COO(C₁₋₅ alkyl), —O—CO(C₁₋₅ alkyl), —CO—NH₂, —CO—NH(C₁₋₅ alkyl), —CO—N(C₁₋₅ alkyl)(C₁₋₅ alkyl), —NH—CO(C₁₋₅ alkyl), —N(C₁₋₅ alkyl)-CO(C₁₋₅ alkyl), —NH—COO(C₁₋₅ alkyl), —N(C₁₋₅ alkyl)-COO(C₁₋₅ alkyl), —O—CO—NH(C₁₋₅ alkyl), —O—CO—N(C₁₋₅ alkyl)(C₁₋₅ alkyl), —SO₂—NH₂, —SO₂—NH(C₁₋₅ alkyl), —SO₂—N(C₁₋₅ alkyl)(C₁₋₅ alkyl), —NH—SO₂—(C₁₋₅ alkyl), —N(C₁₋₅ alkyl)-SO₂—(C₁₋₅ alkyl), —SO₂—(C₁₋₅ alkyl), —SO—(C₁₋₅ alkyl), aryl, heteroaryl, cycloalkyl, heterocycloalkyl, and -L^{X}-R^{X}, wherein said aryl, said heteroaryl, said cycloalkyl, and said heterocycloalkyl are each optionally substituted with one or more groups independently selected from C₁₋₅ alkyl, C₂₋₅ alkenyl, C₂₋₅ alkynyl, halogen, C₁₋₅ haloalkyl, —O(C₁₋₅ haloalkyl), —CN, —OH, —O(C₁₋₅ alkyl), —SH, —S(C₁₋₅ alkyl), —NH₂, —NH(C₁₋₅ alkyl), and —N(C₁₋₅ alkyl)(C₁₋₅ alkyl);
each L^{X} is independently selected from a bond, C₁₋₅ alkylene, C₂₋₅ alkenylene, and C₂₋₅ alkynylene, wherein said alkylene, said alkenylene and said alkynylene are each optionally substituted with one or more groups independently selected from halogen, C₁₋₅ haloalkyl, —CN, —OH, —O(C₁₋₅ alkyl), —SH, —S(C₁₋₅ alkyl), —NH₂, —NH(C₁₋₅ alkyl), and —N(C₁₋₅ alkyl)(C₁₋₅ alkyl), and further wherein one or more —CH₂— units comprised in said alkylene, said alkenylene or said alkynylene are each optionally replaced by a group independently selected from —O—, —NH—, —N(C₁₋₅ alkyl)-, —CO—, —S—, —SO—, and —SO₂—; and
each R^{X} is independently selected from —OH, —O(C₁₋₅ alkyl), —O(C₁₋₅ alkylene)-OH, —O(C₁₋₅ alkylene)-O(C₁₋₅ alkyl), —SH, —S(C₁₋₅ alkyl), —S(C₁₋₅ alkylene)-SH, —S(C₁₋₅ alkylene)-S(C₁₋₅ alkyl), —NH₂, —NH(C₁₋₅ alkyl), —N(C₁₋₅ alkyl)(C₁₋₅ alkyl), —NH—OH, —N(C₁₋₅ alkyl)-OH, —NH—O(C₁₋₅ alkyl), —N(C₁₋₅ alkyl)-O(C₁₋₅ alkyl), halogen, C₁₋₅ haloalkyl, —O(C₁₋₅ haloalkyl), —CN, —NO₂, —CHO, —CO(C₁₋₅ alkyl), —COOH, —COO(C₁₋₅ alkyl), —O—CO(C₁₋₅ alkyl), —CO—NH₂, —CO—NH(C₁₋₅ alkyl), —CO—N(C₁₋₅ alkyl)(C₁₋₅ alkyl), —NH—CO(C₁₋₅ alkyl), —N(C₁₋₅ alkyl)-CO(C₁₋₅ alkyl), —NH—COO(C₁₋₅ alkyl), —N(C₁₋₅ alkyl)-COO(C₁₋₅ alkyl), —O—CO—NH(C₁₋₅ alkyl), —O—CO—N(C₁₋₅ alkyl)(C₁₋₅ alkyl), —SO₂—NH₂, —SO₂—NH(C₁₋₅ alkyl), —SO₂—N(C₁₋₅ alkyl)(C₁₋₅ alkyl), —NH—SO₂—(C₁₋₅ alkyl), —N(C₁₋₅ alkyl)-SO₂—(C₁₋₅ alkyl), —SO₂—(C₁₋₅ alkyl), —SO—(C₁₋₅ alkyl), aryl, heteroaryl, cycloalkyl, and heterocycloalkyl, wherein said aryl, said heteroaryl, said cycloalkyl, and said heterocycloalkyl are each optionally substituted with one or more groups independently selected from C₁₋₅ alkyl, C₂₋₅ alkenyl, C₂₋₅ alkynyl, halogen, C₁₋₅ haloalkyl, —O(C₁₋₅ haloalkyl), —CN, —OH, —O(C₁₋₅ alkyl), —SH, —S(C₁₋₅ alkyl), —NH₂, —NH(C₁₋₅ alkyl), and —N(C₁₋₅ alkyl)(C₁₋₅ alkyl);
or a pharmaceutically acceptable salt or solvate thereof;
wherein the following compounds are excluded from formula (I):
1-(((5-phenyl-4,5-dihydro-1H-imidazol-2-yl)thio)methyl)pyrrolidine;
1-(2-((5-phenyl-4,5-dihydro-1H-imidazol-2-yl)thio)ethyl)pyrrolidine;
1-(3-((5-phenyl-4,5-dihydro-1H-imidazol-2-yl)thio)propyl)pyrrolidine;
1-(((5-phenyl-4,5-dihydro-1H-imidazol-2-yl)thio)methyl)piperidine;
1-(2-((5-phenyl-4,5-dihydro-1H-imidazol-2-yl)thio)ethyl)piperidine;
1-(3-((5-phenyl-4,5-dihydro-1H-imidazol-2-yl)thio)propyl)piperidine;
1-(2-((4,5-dihydro-1H-imidazol-2-yl)thio)ethyl)pyrrolidine;
1-(3-((4,5-dihydro-1H-imidazol-2-yl)thio)propyl)pyrrolidine;
1-(4-((4,5-dihydro-1H-imidazol-2-yl)thio)butyl)pyrrolidine;
1-(2-((4,5-dihydro-1H-imidazol-2-yl)thio)ethyl)piperidine;
1-(3-((4,5-dihydro-1H-imidazol-2-yl)thio)propyl)piperidine;
1-(4-((4,5-dihydro-1H-imidazol-2-yl)thio)butyl)piperidine;
1-(2-((4,5-dihydro-1H-imidazol-2-yl)thio)ethyl)azepane;
1-(3-((4,5-dihydro-1H-imidazol-2-yl)thio)propyl)azepane;
1-(4-((4,5-dihydro-1H-imidazol-2-yl)thio)butyl)azepane;
1-(2-((1,4,5,6-tetrahydropyrimidin-2-yl)thio)ethyl)pyrrolidine;
1-(3-((1,4,5,6-tetrahydropyrimidin-2-yl)thio)propyl)pyrrolidine;
1-(4-((1,4,5,6-tetrahydropyrimidin-2-yl)thio)butyl)pyrrolidine;
1-(2-((1,4,5,6-tetrahydropyrimidin-2-yl)thio)ethyl)piperidine;
1-(3-((1,4,5,6-tetrahydropyrimidin-2-yl)thio)propyl)piperidine;
1-(4-((1,4,5,6-tetrahydropyrimidin-2-yl)thio)butyl)piperidine;
1-(2-((1,4,5,6-tetrahydropyrimidin-2-yl)thio)ethyl)azepane;
1-(3-((1,4,5,6-tetrahydropyrimidin-2-yl)thio)propyl)azepane;
1-(4-((1,4,5,6-tetrahydropyrimidin-2-yl)thio)butyl)azepane;
1-(2-((4,5,6,7-tetrahydro-1H-1,3-diazepin-2-yl)thio)ethyl)pyrrolidine;
1-(3-((4,5,6,7-tetrahydro-1H-1,3-diazepin-2-yl)thio)propyl)pyrrolidine;
1-(4-((4,5,6,7-tetrahydro-1H-1,3-diazepin-2-yl)thio)butyl)pyrrolidine;
1-(2-((4,5,6,7-tetrahydro-1H-1,3-diazepin-2-yl)thio)ethyl)piperidine;
1-(3-((4,5,6,7-tetrahydro-1H-1,3-diazepin-2-yl)thio)propyl)piperidine;
1-(4-((4,5,6,7-tetrahydro-1H-1,3-diazepin-2-yl)thio)butyl)piperidine;
1-(2-((4,5,6,7-tetrahydro-1H-1,3-diazepin-2-yl)thio)ethyl)azepane;
1-(3-((4,5,6,7-tetrahydro-1H-1,3-diazepin-2-yl)thio)propyl)azepane;
1-(4-((4,5,6,7-tetrahydro-1H-1,3-diazepin-2-yl)thio)butyl)azepane;
2-((4,5-dihydro-1H-imidazol-2-yl)thio)-1-(pyridin-4-yl)ethan-1-one;
3-(1-(3-((4,5-dihydro-1H-imidazol-2-yl)thio)propyl)-1H-indol-3-yl)-4-(1-methyl-1H-indol-3-yl)-1H-pyrrole-2,5-dione;
2-((4,5-dihydro-1H-imidazol-2-yl)thio)-1-(pyridin-4-yl)ethanone;
3-((1,4,5,6-tetrahydropyrimidin-2-yl)thio)-1H-pyrrolo[2,3-b]pyridine;
3-((3,4-dihydroquinazolin-2-yl)thio)-1H-indole-2-carboxylic acid;
2-(cyclopentylthio)-4,5-dihydro-1H-imidazole;
N-(piperidinomethyl)-2-[(piperidinomethyl)thio]-2-imidazoline;
N-((2-methylpiperidino)methyl)-2-[((2-methylpiperidino)methyl)thio]-2-imidazoline;
N-((3-methylpiperidino)methyl)-2-[((3-methylpiperidino)methyl)thio]-2-imidazoline;
N-((4-methylpiperidino)methyl)-2-[((4-methylpiperidino)methyl)thio]-2-imidazoline; and
N-((2-methyl-5-ethylpiperidino)methyl)-2-[((2-methyl-5-ethylpiperidino)methyl)thio]-2-imidazoline.

2. The compound of claim 1, wherein ring A is a group A1 which is selected from the following groups A1a, A1b and A1c: 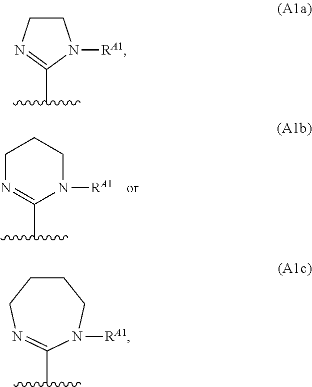
wherein ring A is optionally substituted with one or more groups R^{A2};
wherein it is preferred that ring A is a group A1a 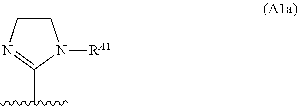
which is optionally substituted with one or more groups R^{A2}.

3. The compound of claim 1, wherein ring A is a group A2 which is selected from the following groups A2a, A2b, A2c, A2d and A2e: 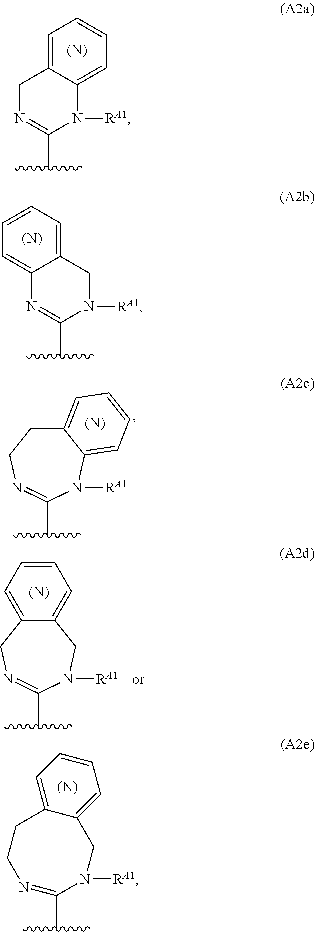
wherein the symbol “(N)” depicted inside a ring indicates that 0, 1, 2 or 3 ring atom(s) of the respective ring is/are nitrogen ring atom(s); and
wherein ring A is optionally substituted with one or more groups R^{A2}.

4. The compound of claim 1, wherein ring A is a group A2 which is selected from the following groups A2a1, A2a2, A2b1, A2c1, A2d1 and A2e1: 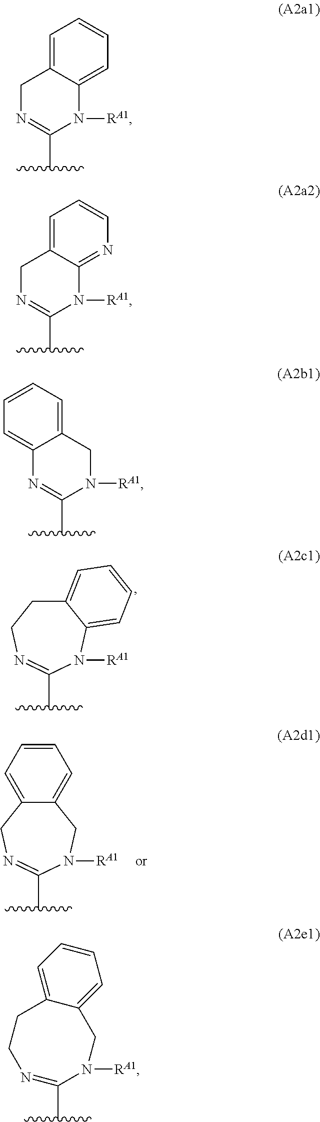
wherein ring A is optionally substituted with one or more groups R^{A2};
wherein it is preferred that ring A is a group A2a1 or A2c1 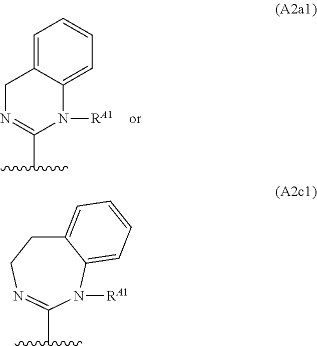
which is optionally substituted with one or more groups R^{A2}.

5. The compound of any one of claims 1 to 4, wherein R^{A1} is selected from hydrogen, C₁₋₅ alkyl, and cycloalkyl, wherein said cycloalkyl is optionally substituted with one or more groups R^{Cyc}.

6. The compound of any one of claims 1 to 5, wherein each R^{A2} is independently selected from C₁₋₅ alkyl, C₂₋₅ alkenyl, C₂₋₅ alkynyl, halogen, C₁₋₅ haloalkyl, —(C₀₋₅ alkylene)-O(C₁₋₅ haloalkyl), —(C₀₋₅ alkylene)-CN, —(C₀₋₅ alkylene)-OH, —(C₀₋₅ alkylene)-O(C₁₋₅ alkyl), —(C₀₋₅ alkylene)-O(C₁₋₅ alkylene)-OH, —(C₀₋₅ alkylene)-O(C₁₋₅ alkylene)-O(C₁₋₅ alkyl), —(C₀₋₅ alkylene)-NH₂, —(C₀₋₅ alkylene)-NH(C₁₋₅ alkyl), —(C₀₋₅ alkylene)-N(C₁₋₅ alkyl)(C₁₋₅ alkyl), —(C₀₋₅ alkylene)-CHO, —(C₀₋₅ alkylene)-CO(C₁₋₅ alkyl), —(C₀₋₅ alkylene)-COOH, —(C₀₋₅ alkylene)-COO(C₁₋₅ alkyl), —(C₀₋₅ alkylene)-O—CO(C₁₋₅ alkyl), —(C₀₋₅ alkylene)-CO—NH₂, —(C₀₋₅ alkylene)-CO—NH(C₁₋₅ alkyl), —(C₀₋₅ alkylene)-CO—N(C₁₋₅ alkyl)(C₁₋₅ alkyl), —(C₀₋₅ alkylene)-NH—CO(C₁₋₅ alkyl), —(C₀₋₅ alkylene)-N(C₁₋₅ alkyl)-CO(C₁₋₅ alkyl), —(C₀₋₅ alkylene)-SH, —(C₀₋₅ alkylene)-S(C₁₋₅ alkyl), —(C₀₋₅ alkylene)-SO—(C₁₋₅ alkyl), —(C₀₋₅ alkylene)-SO₂—(C₁₋₅ alkyl), —(C₀₋₅ alkylene)-SO₂—NH₂, —(C₀₋₅ alkylene)-SO₂—NH(C₁₋₅ alkyl), —(C₀₋₅ alkylene)-SO₂—N(C₁₋₅ alkyl)(C₁₋₅ alkyl), —(C₀₋₅ alkylene)-NH—SO₂—(C₁₋₅ alkyl), —(C₀₋₅ alkylene)-N(C₁₋₅ alkyl)-SO₂—(C₁₋₅ alkyl), —(C₀₋₅ alkylene)-cycloalkyl, —(C₀₋₅ alkylene)-aryl, —(C₀₋₅ alkylene)-heterocycloalkyl, and —(C₀₋₅ alkylene)-heteroaryl, wherein the cycloalkyl moiety in said —(C₀₋₅ alkylene)-cycloalkyl, the aryl moiety in said —(C₀₋₅ alkylene)-aryl, the heterocycloalkyl moiety in said —(C₀₋₅ alkylene)-heterocycloalkyl, and the heteroaryl moiety in said —(C₀₋₅ alkylene)-heteroaryl are each optionally substituted with one or more groups R^{Cyc}.

7. The compound of any one of claims 1 to 6, wherein n is 0, and further wherein L is a covalent bond, —CH₂— or —CH₂C(═O)—, wherein said —CH₂C(═O)— is attached via its C(═O) carbon atom to ring B.

8. The compound of any one of claims 1 to 7, wherein ring B is a group 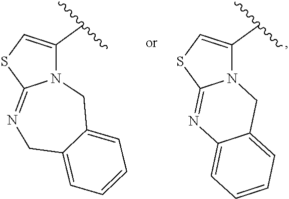 [SMILES: *C1=CSC2=NCc3ccccc3CN12 ; *C1=CSC2=Nc3ccccc3CN12]
which is optionally substituted with one or more groups R^{B1}.

9. The compound of any one of claims 1 or 3 to 7, wherein ring A is a group A2, A3, A4, A5, A6, A7, A8, A9, A10 or A11, and ring B is a group 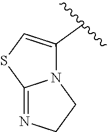 [SMILES: *C1=CSC2=NCCN12]
which is optionally substituted with one or more groups R^{B1}.

10. The compound of claim 1, which is a compound of formula (I)
wherein: 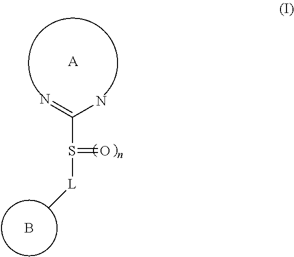
ring A is any one of the following groups A1 or A2: 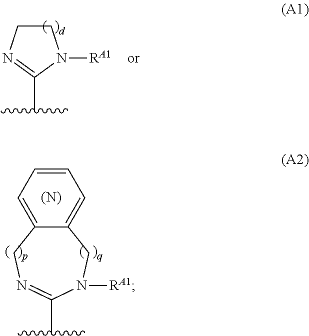
wherein d is 1, 2 or 3;
wherein p is 0, 1, 2 or 3, and q is 0, 1 or 2, with the proviso that p and q are not both 0;
wherein the symbol “(N)” depicted inside a ring indicates that 0, 1, 2 or 3 ring atom(s) of the respective ring is/are nitrogen ring atom(s); and
wherein ring A is optionally substituted with one or more groups R^{A2};
n is 0, 1 or 2;
L is a covalent bond or C₁₋₅ alkylene, wherein said alkylene is optionally substituted with one or more groups R^{L}, wherein one or more —CH₂— units comprised in said alkylene are each optionally replaced by a group independently selected from —O—, —NH—, —N(C₁₋₅ alkyl)-, —CO—, —S—, —SO—, —SO₂—, carbocyclylene, and heterocyclylene, and wherein each R^{L} is independently selected from —OH, —O(C₁₋₅ alkyl), —SH, —S(C₁₋₅ alkyl), —NH₂, —NH(C₁₋₅ alkyl), —N(C₁₋₅ alkyl)(C₁₋₅ alkyl), halogen, —CF₃, —CN, C₁₋₅ alkyl, cycloalkyl, and heterocycloalkyl;
if ring A is a group A1, then ring B is selected from any one of the following groups: 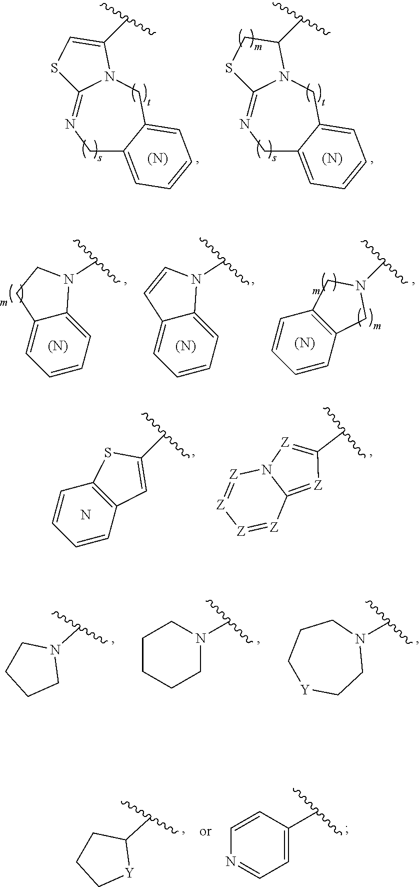
wherein each of the above-depicted groups is optionally substituted with one or more groups R^{B1};
wherein each s is independently 0, 1 or 2;
wherein each t is independently 0, 1, 2 or 3;
wherein each m is independently 1, 2 or 3;
wherein each ring atom Y is independently selected from S, O, SO₂, NH and CH₂;
wherein each ring atom Z is independently C or N;
wherein the symbol “(N)” depicted inside a ring indicates that 0, 1, 2 or 3 ring atom(s) of the respective ring is/are nitrogen ring atom(s); and
wherein the symbol “N” depicted inside a ring indicates that 1, 2 or 3 ring atom(s) of the respective ring is/are nitrogen ring atom(s);
if ring A is a group A2, then ring B is selected from any one of the following groups: 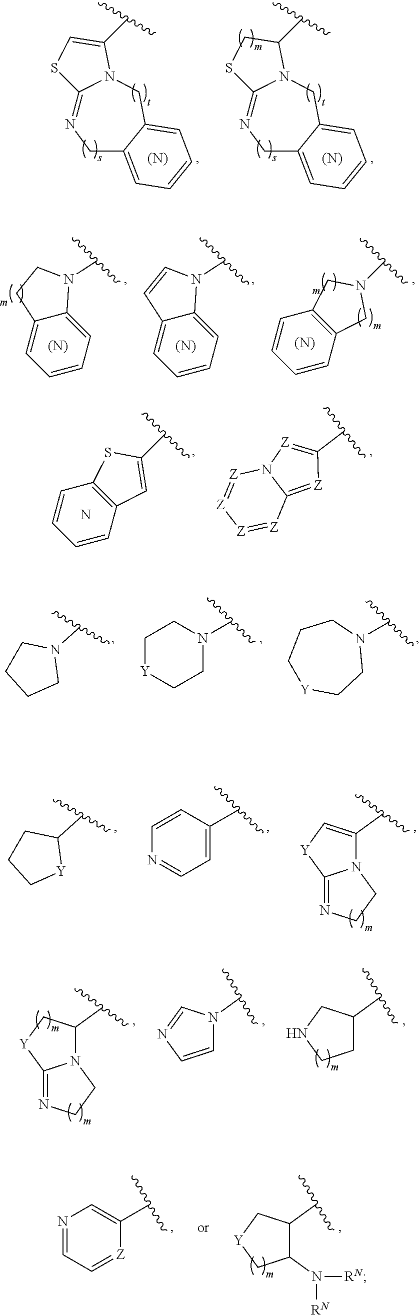
wherein each of the above-depicted groups is optionally substituted with one or more groups R^{B1};
wherein each s is independently 0, 1 or 2;
wherein each t is independently 0, 1, 2 or 3;
wherein each m is independently 1, 2 or 3;
wherein each ring atom Y is independently selected from S, O, SO₂, NH and CH₂;
wherein each ring atom Z is independently C or N;
wherein the symbol “(N)” depicted inside a ring indicates that 0, 1, 2 or 3 ring atom(s) of the respective ring is/are nitrogen ring atom(s); and
wherein the symbol “N” depicted inside a ring indicates that 1, 2 or 3 ring atom(s) of the respective ring is/are nitrogen ring atom(s);
R^{A1} is selected from hydrogen, C₁₋₅ alkyl, C₂₋₅ alkenyl, C₂₋₅ alkynyl, —CO(C₁₋₅ alkyl), —COO(C₁₋₅ alkyl), carbocyclyl, and heterocyclyl, wherein said alkyl, said alkenyl, said alkynyl, the alkyl moiety in said —CO(C₁₋₅ alkyl), and the alkyl moiety in said —COO(C₁₋₅ alkyl) are each optionally substituted with one or more groups R^{Alk}, and further wherein said carbocyclyl and said heterocyclyl are each optionally substituted with one or more groups R^{Cyc};
each R^{A2} is independently selected from C₁₋₅ alkyl, C₂₋₅ alkenyl, C₂₋₅ alkynyl, —(C₀₋₅ alkylene)-R^{A21}, —(C₂₋₅ alkenylene)-R^{A21}, and —(C₂₋₅ alkynylene)-R^{A21}, wherein said alkyl, said alkenyl, said alkynyl, said alkylene, said alkenylene, and said alkynylene are each optionally substituted with one or more groups R^{Alk}, and further wherein one or more —CH₂— units comprised in said alkylene, said alkenylene, or said alkynylene are each optionally replaced by a group independently selected from —O—, —NH—, —N(C₁₋₅ alkyl)-, —CO—, —S—, —SO—, and —SO₂—;
wherein any two groups R^{A2}, which are attached to the same ring atom of ring A, may also be mutually joined to form, together with the ring atom that they are attached to, a cycloalkyl or a heterocycloalkyl, wherein said cycloalkyl or said heterocycloalkyl is optionally substituted with one or more groups R^{Cyc};
wherein any two groups R^{A2}, which are attached to distinct ring atoms of ring A, may also be mutually joined to form a C₁₋₅ alkylene which is optionally substituted with one or more groups R^{Cyc}, and wherein one or more —CH₂— units comprised in said alkylene are each optionally replaced by a group independently selected from —O—, —NH—, —N(C₁₋₅ alkyl)-, —CO—, —S—, —SO—, —SO₂—, and phen-1,2-diyl, wherein said phen-1,2-diyl is optionally substituted with one or more groups R^{Cyc}; and
wherein any one group R^{A2} may also be mutually joined with R^{A1} to form a C₁₋₅ alkylene which is optionally substituted with one or more groups R^{Cyc}, and wherein one or more —CH₂— units comprised in said alkylene are each optionally replaced by a group independently selected from —O—, —NH—, —N(C₁₋₅ alkyl)-, —CO—, —S—, —SO—, —SO₂—, and phen-1,2-diyl, wherein said phen-1,2-diyl is optionally substituted with one or more groups R^{Cyc};
each R^{A21} is independently selected from halogen, C₁₋₅ haloalkyl, —O(C₁₋₅ haloalkyl), —CN, —OR^{A22}, —NR^{A22}R^{A22}, —NR^{A22}OR^{A22}, —COR^{A22}, —COOR^{A22}, —OCOR^{A22}, —CONR^{A22}R^{A22}, —NR^{A22}COR^{A22}, —NR^{A22}COOR^{A22}, —OCONR^{A22}R^{A22}, —SR^{A22}, —SOR^{A22}, —SO₂R^{A22}, —SO₂NR^{A22}R^{A22}, —NR^{A22}SO₂R^{A22}, —SO₃R^{A22}, —NO₂, carbocyclyl, and heterocyclyl, wherein said carbocyclyl and said heterocyclyl are each optionally substituted with one or more groups R^{Cyc};
each R^{A22} is independently selected from hydrogen, C₁₋₅ alkyl, C₂₋₅ alkenyl, C₂₋₅ alkynyl, carbocyclyl, and heterocyclyl, wherein said alkyl, said alkenyl and said alkynyl are each optionally substituted with one or more groups R^{Alk}, and further wherein said carbocyclyl and said heterocyclyl are each optionally substituted with one or more groups R^{Cyc};
each R^{N} is independently selected from hydrogen, C₁₋₅ alkyl, C₂₋₅ alkenyl, C₂₋₅ alkynyl, —O(C₁₋₅ alkyl), —CO(C₁₋₅ alkyl), —COO(C₁₋₅ alkyl), carbocyclyl, and heterocyclyl, wherein said alkyl, said alkenyl, said alkynyl, the alkyl moiety in said —O(C₁₋₅ alkyl), the alkyl moiety in said —CO(C₁₋₅ alkyl), and the alkyl moiety in said —COO(C₁₋₅ alkyl) are each optionally substituted with one or more groups R^{Alk}, wherein said carbocyclyl and said heterocyclyl are each optionally substituted with one or more groups R^{Cyc}, and further wherein any two groups R^{N} which are attached to the same nitrogen atom may also be mutually joined to form, together with the nitrogen atom that they are attached to, a heterocyclyl which is optionally substituted with one or more groups R^{Cyc};
each R^{B1} is independently selected from C₁₋₅ alkyl, C₂₋₅ alkenyl, C₂₋₅ alkynyl, —(C₀₋₅ alkylene)-R^{B11}, —(C₂₋₅ alkenylene)-R^{B11}, —(C₂₋₅ alkynylene)-R^{B11}, and ═R^{B13}, wherein said alkyl, said alkenyl, said alkynyl, said alkylene, said alkenylene, and said alkynylene are each optionally substituted with one or more groups R^{Alk}, and further wherein one or more —CH₂— units comprised in said alkylene, said alkenylene, or said alkynylene are each optionally replaced by a group independently selected from —O—, —NH—, —N(C₁₋₅ alkyl)-, —CO—, —S—, —SO—, and —SO₂—;
wherein any two groups R^{B1}, which are attached to the same ring atom of ring B, may also be mutually joined to form, together with the ring atom that they are attached to, a cycloalkyl or a heterocycloalkyl, wherein said cycloalkyl or said heterocycloalkyl is optionally substituted with one or more groups R^{Cyc}; and
wherein any two groups R^{B1}, which are attached to distinct ring atoms of ring B, may also be mutually joined to form a C₁₋₅ alkylene which is optionally substituted with one or more groups R^{Cyc}, and wherein one or more —CH₂— units comprised in said alkylene are each optionally replaced by a group independently selected from —O—, —NH—, —N(C₁₋₅ alkyl)-, —CO—, —S—, —SO—, and —SO₂—;
each R^{B11} is independently selected from halogen, C₁₋₅ haloalkyl, —O(C₁₋₅ haloalkyl), —CN, —OR^{B12}, —NR^{B12}R^{B12}, —N⁺R^{B12}R^{B12}R^{B12}, —NR^{B12}OR^{B12}, —COR^{B12}, —COOR^{B12}, —OCOR^{B12}, —CONR^{B12}R^{B12}, —NR^{B12}COR^{B12}, —NR^{B12}COOR^{B12}, —OCONR^{B12}R^{B12}, —SR^{B12}, —SOR^{B12}, —SO₂R^{B12}, —SO₂NR^{B12}R^{B12}, —NR^{B12}SO₂R^{B12}, —SO₃R^{B12}, —NO₂, carbocyclyl, and heterocyclyl, wherein said carbocyclyl and said heterocyclyl are each optionally substituted with one or more groups R^{Cyc};
each R^{B12} is independently selected from hydrogen, C₁₋₅ alkyl, C₂₋₅ alkenyl, C₂₋₅ alkynyl, carbocyclyl, and heterocyclyl, wherein said alkyl, said alkenyl and said alkynyl are each optionally substituted with one or more groups R^{Alk}, and further wherein said carbocyclyl and said heterocyclyl are each optionally substituted with one or more groups R^{Cyc};
each R^{B13} is independently selected from ═O, ═S, and ═N—R^{B12};
each R^{Alk} is independently selected from —OH, —O(C₁₋₅ alkyl), —O(C₁₋₅ alkylene)-OH, —O(C₁₋₅ alkylene)-O(C₁₋₅ alkyl), —SH, —S(C₁₋₅ alkyl), —S(C₁₋₅ alkylene)-SH, —S(C₁₋₅ alkylene)-S(C₁₋₅ alkyl), —NH₂, —NH(C₁₋₅ alkyl), —N(C₁₋₅ alkyl)(C₁₋₅ alkyl), —NH—OH, —N(C₁₋₅ alkyl)-OH, —NH—O(C₁₋₅ alkyl), —N(C₁₋₅ alkyl)-O(C₁₋₅ alkyl), halogen, C₁₋₅ haloalkyl, —O(C₁₋₅ haloalkyl), —CN, —NO₂, —CHO, —CO(C₁₋₅ alkyl), —COOH, —COO(C₁₋₅ alkyl), —O—CO(C₁₋₅ alkyl), —CO—NH₂, —CO—NH(C₁₋₅ alkyl), —CO—N(C₁₋₅ alkyl)(C₁₋₅ alkyl), —NH—CO(C₁₋₅ alkyl), —N(C₁₋₅ alkyl)-CO(C₁₋₅ alkyl), —NH—COO(C₁₋₅ alkyl), —N(C₁₋₅ alkyl)-COO(C₁₋₅ alkyl), —O—CO—NH(C₁₋₅ alkyl), —O—CO—N(C₁₋₅ alkyl)(C₁₋₅ alkyl), —SO₂—NH₂, —SO₂—NH(C₁₋₅ alkyl), —SO₂—N(C₁₋₅ alkyl)(C₁₋₅ alkyl), —NH—SO₂—(C₁₋₅ alkyl), —N(C₁₋₅ alkyl)-SO₂—(C₁₋₅ alkyl), —SO₂—(C₁₋₅ alkyl), —SO—(C₁₋₅ alkyl), aryl, heteroaryl, cycloalkyl, heterocycloalkyl, and -L^{X}-R^{X}, wherein said aryl, said heteroaryl, said cycloalkyl, and said heterocycloalkyl are each optionally substituted with one or more groups independently selected from C₁₋₅ alkyl, C₂₋₅ alkenyl, C₂₋₅ alkynyl, halogen, C₁₋₅ haloalkyl, —O(C₁₋₅ haloalkyl), —CN, —OH, —O(C₁₋₅ alkyl), —SH, —S(C₁₋₅ alkyl), —NH₂, —NH(C₁₋₅ alkyl), and —N(C₁₋₅ alkyl)(C₁₋₅ alkyl);
each R^{Cyc} is independently selected from C₁₋₅ alkyl, C₂₋₅ alkenyl, C₂₋₅ alkynyl, —OH, —O(C₁₋₅ alkyl), —O(C₁₋₅ alkylene)-OH, —O(C₁₋₅ alkylene)-O(C₁₋₅ alkyl), —SH, —S(C₁₋₅ alkyl), —S(C₁₋₅ alkylene)-SH, —S(C₁₋₅ alkylene)-S(C₁₋₅ alkyl), —NH₂, —NH(C₁₋₅ alkyl), —N(C₁₋₅ alkyl)(C₁₋₅ alkyl), —NH—OH, —N(C₁₋₅ alkyl)-OH, —NH—O(C₁₋₅ alkyl), —N(C₁₋₅ alkyl)-O(C₁₋₅ alkyl), halogen, C₁₋₅ haloalkyl, —O(C₁₋₅ haloalkyl), —CN, —NO₂, —CHO, —CO(C₁₋₅ alkyl), —COOH, —COO(C₁₋₅ alkyl), —O—CO(C₁₋₅ alkyl), —CO—NH₂, —CO—NH(C₁₋₅ alkyl), —CO—N(C₁₋₅ alkyl)(C₁₋₅ alkyl), —NH—CO(C₁₋₅ alkyl), —N(C₁₋₅ alkyl)-CO(C₁₋₅ alkyl), —NH—COO(C₁₋₅ alkyl), —N(C₁₋₅ alkyl)-COO(C₁₋₅ alkyl), —O—CO—NH(C₁₋₅ alkyl), —O—CO—N(C₁₋₅ alkyl)(C₁₋₅ alkyl), —SO₂—NH₂, —SO₂—NH(C₁₋₅ alkyl), —SO₂—N(C₁₋₅ alkyl)(C₁₋₅ alkyl), —NH—SO₂—(C₁₋₅ alkyl), —N(C₁₋₅ alkyl)-SO₂—(C₁₋₅ alkyl), —SO₂—(C₁₋₅ alkyl), —SO—(C₁₋₅ alkyl), aryl, heteroaryl, cycloalkyl, heterocycloalkyl, and -L^{X}-R^{X}, wherein said aryl, said heteroaryl, said cycloalkyl, and said heterocycloalkyl are each optionally substituted with one or more groups independently selected from C₁₋₅ alkyl, C₂₋₅ alkenyl, C₂₋₅ alkynyl, halogen, C₁₋₅ haloalkyl, —O(C₁₋₅ haloalkyl), —CN, —OH, —O(C₁₋₅ alkyl), —SH, —S(C₁₋₅ alkyl), —NH₂, —NH(C₁₋₅ alkyl), and —N(C₁₋₅ alkyl)(C₁₋₅ alkyl);
each L^{X} is independently selected from a bond, C₁₋₅ alkylene, C₂₋₅ alkenylene, and C₂₋₅ alkynylene, wherein said alkylene, said alkenylene and said alkynylene are each optionally substituted with one or more groups independently selected from halogen, C₁₋₅ haloalkyl, —CN, —OH, —O(C₁₋₅ alkyl), —SH, —S(C₁₋₅ alkyl), —NH₂, —NH(C₁₋₅ alkyl), and —N(C₁₋₅ alkyl)(C₁₋₅ alkyl), and further wherein one or more —CH₂— units comprised in said alkylene, said alkenylene or said alkynylene are each optionally replaced by a group independently selected from —O—, —NH—, —N(C₁₋₅ alkyl)-, —CO—, —S—, —SO—, and —SO₂—; and
each R^{X} is independently selected from —OH, —O(C₁₋₅ alkyl), —O(C₁₋₅ alkylene)-OH, —O(C₁₋₅ alkylene)-O(C₁₋₅ alkyl), —SH, —S(C₁₋₅ alkyl), —S(C₁₋₅ alkylene)-SH, —S(C₁₋₅ alkylene)-S(C₁₋₅ alkyl), —NH₂, —NH(C₁₋₅ alkyl), —N(C₁₋₅ alkyl)(C₁₋₅ alkyl), —NH—OH, —N(C₁₋₅ alkyl)-OH, —NH—O(C₁₋₅ alkyl), —N(C₁₋₅ alkyl)-O(C₁₋₅ alkyl), halogen, C₁₋₅ haloalkyl, —O(C₁₋₅ haloalkyl), —CN, —NO₂, —CHO, —CO(C₁₋₅ alkyl), —COOH, —COO(C₁₋₅ alkyl), —O—CO(C₁₋₅ alkyl), —CO—NH₂, —CO—NH(C₁₋₅ alkyl), —CO—N(C₁₋₅ alkyl)(C₁₋₅ alkyl), —NH—CO(C₁₋₅ alkyl), —N(C₁₋₅ alkyl)-CO(C₁₋₅ alkyl), —NH—COO(C₁₋₅ alkyl), —N(C₁₋₅ alkyl)-COO(C₁₋₅ alkyl), —O—CO—NH(C₁₋₅ alkyl), —O—CO—N(C₁₋₅ alkyl)(C₁₋₅ alkyl), —SO₂—NH₂, —SO₂—NH(C₁₋₅ alkyl), —SO₂—N(C₁₋₅ alkyl)(C₁₋₅ alkyl), —NH—SO₂—(C₁₋₅ alkyl), —N(C₁₋₅ alkyl)-SO₂—(C₁₋₅ alkyl), —SO₂—(C₁₋₅ alkyl), —SO—(C₁₋₅ alkyl), aryl, heteroaryl, cycloalkyl, and heterocycloalkyl, wherein said aryl, said heteroaryl, said cycloalkyl, and said heterocycloalkyl are each optionally substituted with one or more groups independently selected from C₁₋₅ alkyl, C₂₋₅ alkenyl, C₂₋₅ alkynyl, halogen, C₁₋₅ haloalkyl, —O(C₁₋₅ haloalkyl), —CN, —OH, —O(C₁₋₅ alkyl), —SH, —S(C₁₋₅ alkyl), —NH₂, —NH(C₁₋₅ alkyl), and —N(C₁₋₅ alkyl)(C₁₋₅ alkyl);
or a pharmaceutically acceptable salt or solvate thereof;
wherein the following compounds are excluded from formula (I):
1-(((5-phenyl-4,5-dihydro-1H-imidazol-2-yl)thio)methyl)pyrrolidine;
1-(2-((5-phenyl-4,5-dihydro-1H-imidazol-2-yl)thio)ethyl)pyrrolidine;
1-(3-((5-phenyl-4,5-dihydro-1H-imidazol-2-yl)thio)propyl)pyrrolidine;
1-(((5-phenyl-4,5-dihydro-1H-imidazol-2-yl)thio)methyl)piperidine;
1-(2-((5-phenyl-4,5-dihydro-1H-imidazol-2-yl)thio)ethyl)piperidine;
1-(3-((5-phenyl-4,5-dihydro-1H-imidazol-2-yl)thio)propyl)piperidine;
1-(2-((4,5-dihydro-1H-imidazol-2-yl)thio)ethyl)pyrrolidine;
1-(3-((4,5-dihydro-1H-imidazol-2-yl)thio)propyl)pyrrolidine;
1-(4-((4,5-dihydro-1H-imidazol-2-yl)thio)butyl)pyrrolidine;
1-(2-((4,5-dihydro-1H-imidazol-2-yl)thio)ethyl)piperidine;
1-(3-((4,5-dihydro-1H-imidazol-2-yl)thio)propyl)piperidine;
1-(4-((4,5-dihydro-1H-imidazol-2-yl)thio)butyl)piperidine;
1-(2-((4,5-dihydro-1H-imidazol-2-yl)thio)ethyl)azepane;
1-(3-((4,5-dihydro-1H-imidazol-2-yl)thio)propyl)azepane;
1-(4-((4,5-dihydro-1H-imidazol-2-yl)thio)butyl)azepane;
1-(2-((1,4,5,6-tetrahydropyrimidin-2-yl)thio)ethyl)pyrrolidine;
1-(3-((1,4,5,6-tetrahydropyrimidin-2-yl)thio)propyl)pyrrolidine;
1-(4-((1,4,5,6-tetrahydropyrimidin-2-yl)thio)butyl)pyrrolidine;
1-(2-((1,4,5,6-tetrahydropyrimidin-2-yl)thio)ethyl)piperidine;
1-(3-((1,4,5,6-tetrahydropyrimidin-2-yl)thio)propyl)piperidine;
1-(4-((1,4,5,6-tetrahydropyrimidin-2-yl)thio)butyl)piperidine;
1-(2-((1,4,5,6-tetrahydropyrimidin-2-yl)thio)ethyl)azepane;
1-(3-((1,4,5,6-tetrahydropyrimidin-2-yl)thio)propyl)azepane;
1-(4-((1,4,5,6-tetrahydropyrimidin-2-yl)thio)butyl)azepane;
1-(2-((4,5,6,7-tetrahydro-1H-1,3-diazepin-2-yl)thio)ethyl)pyrrolidine;
1-(3-((4,5,6,7-tetrahydro-1H-1,3-diazepin-2-yl)thio)propyl)pyrrolidine;
1-(4-((4,5,6,7-tetrahydro-1H-1,3-diazepin-2-yl)thio)butyl)pyrrolidine;
1-(2-((4,5,6,7-tetrahydro-1H-1,3-diazepin-2-yl)thio)ethyl)piperidine;
1-(3-((4,5,6,7-tetrahydro-1H-1,3-diazepin-2-yl)thio)propyl)piperidine;
1-(4-((4,5,6,7-tetrahydro-1H-1,3-diazepin-2-yl)thio)butyl)piperidine;
1-(2-((4,5,6,7-tetrahydro-1H-1,3-diazepin-2-yl)thio)ethyl)azepane;
1-(3-((4,5,6,7-tetrahydro-1H-1,3-diazepin-2-yl)thio)propyl)azepane;
1-(4-((4,5,6,7-tetrahydro-1H-1,3-diazepin-2-yl)thio)butyl)azepane;
2-((4,5-dihydro-1H-imidazol-2-yl)thio)-1-(pyridin-4-yl)ethan-1-one;
3-(1-(3-((4,5-dihydro-1H-imidazol-2-yl)thio)propyl)-1H-indol-3-yl)-4-(1-methyl-1H-indol-3-yl)-1H-pyrrole-2,5-dione;
2-((4,5-dihydro-1H-imidazol-2-yl)thio)-1-(pyridin-4-yl)ethanone;
2-(cyclopentylthio)-4,5-dihydro-1H-imidazole;
N-(piperidinomethyl)-2-[(piperidinomethyl)thio]-2-imidazoline;
N-((2-methylpiperidino)methyl)-2-[((2-methylpiperidino)methyl)thio]-2-imidazoline;
N-((3-methylpiperidino)methyl)-2-[((3-methylpiperidino)methyl)thio]-2-imidazoline;
N-((4-methylpiperidino)methyl)-2-[((4-methylpiperidino)methyl)thio]-2-imidazoline; and
N-((2-methyl-5-ethylpiperidino)methyl)-2-[((2-methyl-5-ethylpiperidino)methyl)thio]-2-imidazoline.

11. The compound of claim 1, which is a compound of formula (I) 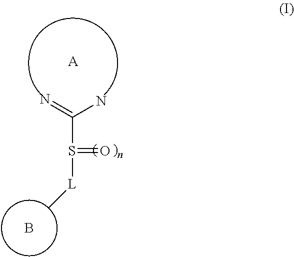
wherein:
ring A is any one of the following groups A1 or A2: 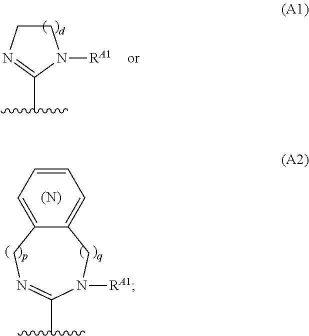
wherein d is 1, 2 or 3;
wherein p is 0, 1, 2 or 3, and q is 0, 1 or 2, with the proviso that p and q are not both 0;
wherein the symbol “(N)” depicted inside a ring indicates that 0, 1, 2 or 3 ring atom(s) of the respective ring is/are nitrogen ring atom(s); and
wherein ring A is optionally substituted with one or more groups R^{A2};
n is 0, 1 or 2;
L is a covalent bond or C₁₋₅ alkylene;
if ring A is a group A1, then ring B is selected from any one of the following groups: 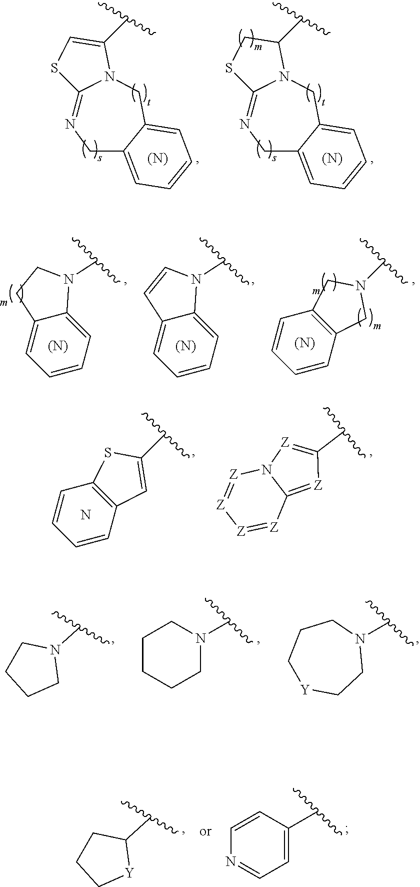
wherein each of the above-depicted groups is optionally substituted with one or more groups R^{B1};
wherein each s is independently 0, 1 or 2;
wherein each t is independently 0, 1, 2 or 3;
wherein each m is independently 1, 2 or 3;
wherein each ring atom Y is independently selected from S, O, SO₂, NH and CH₂;
wherein each ring atom Z is independently C or N;
wherein the symbol “(N)” depicted inside a ring indicates that 0, 1, 2 or 3 ring atom(s) of the respective ring is/are nitrogen ring atom(s); and
wherein the symbol “N” depicted inside a ring indicates that 1, 2 or 3 ring atom(s) of the respective ring is/are nitrogen ring atom(s);
if ring A is a group A2, then ring B is selected from any one of the following groups: 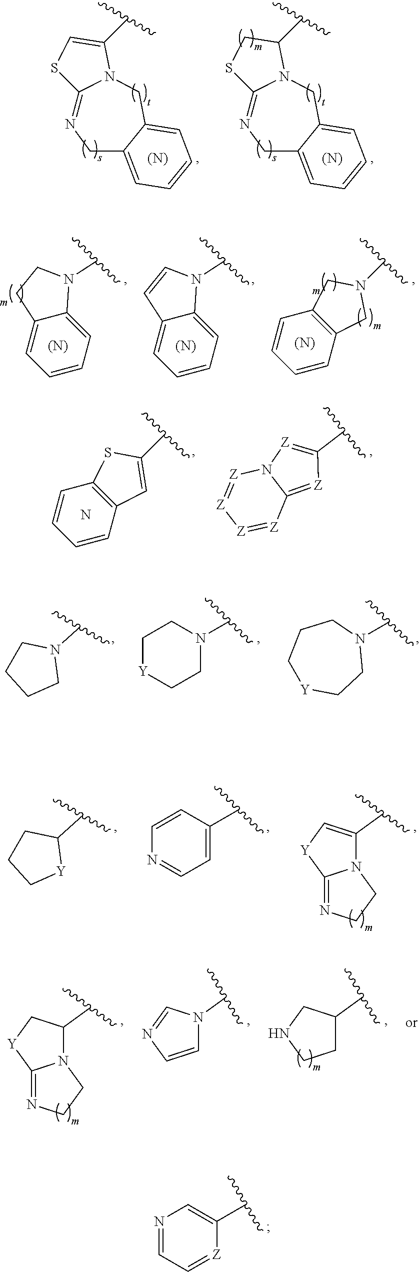
wherein each of the above-depicted groups is optionally substituted with one or more groups R^{B1};
wherein each s is independently 0, 1 or 2;
wherein each t is independently 0, 1, 2 or 3;
wherein each m is independently 1, 2 or 3;
wherein each ring atom Y is independently selected from S, O, SO₂, NH and CH₂;
wherein each ring atom Z is independently C or N;
wherein the symbol “(N)” depicted inside a ring indicates that 0, 1, 2 or 3 ring atom(s) of the respective ring is/are nitrogen ring atom(s); and
wherein the symbol “N” depicted inside a ring indicates that 1, 2 or 3 ring atom(s) of the respective ring is/are nitrogen ring atom(s);
R^{A1} is selected from hydrogen, C₁₋₅ alkyl, C₂₋₅ alkenyl, C₂₋₅ alkynyl, —CO(C₁₋₅ alkyl), —COO(C₁₋₅ alkyl), carbocyclyl, and heterocyclyl, wherein said alkyl, said alkenyl, said alkynyl, the alkyl moiety in said —CO(C₁₋₅ alkyl), and the alkyl moiety in said —COO(C₁₋₅ alkyl) are each optionally substituted with one or more groups R^{Alk}, and further wherein said carbocyclyl and said heterocyclyl are each optionally substituted with one or more groups R^{Cyc};
each R^{A2} is independently selected from C₁₋₅ alkyl, C₂₋₅ alkenyl, C₂₋₅ alkynyl, —(C₀₋₅ alkylene)-R^{A21}, —(C₂₋₅ alkenylene)-R^{A21}, and —(C₂₋₅ alkynylene)-R^{A21}, wherein said alkyl, said alkenyl, said alkynyl, said alkylene, said alkenylene, and said alkynylene are each optionally substituted with one or more groups R^{Alk}, and further wherein one or more —CH₂— units comprised in said alkylene, said alkenylene, or said alkynylene are each optionally replaced by a group independently selected from —O—, —NH—, —N(C₁₋₅ alkyl)-, —CO—, —S—, —SO—, and —SO₂—;
wherein any two groups R^{A2}, which are attached to the same ring atom of ring A, may also be mutually joined to form, together with the ring atom that they are attached to, a cycloalkyl or a heterocycloalkyl, wherein said cycloalkyl or said heterocycloalkyl is optionally substituted with one or more groups R^{Cyc};
wherein any two groups R^{A2}, which are attached to distinct ring atoms of ring A, may also be mutually joined to form a C₁₋₅ alkylene which is optionally substituted with one or more groups R^{Cyc}, and wherein one or more —CH₂— units comprised in said alkylene are each optionally replaced by a group independently selected from —O—, —NH—, —N(C₁₋₅ alkyl)-, —CO—, —S—, —SO—, —SO₂—, and phen-1,2-diyl, wherein said phen-1,2-diyl is optionally substituted with one or more groups R^{Cyc}; and
wherein any one group R^{A2} may also be mutually joined with R^{A1} to form a C₁₋₅ alkylene which is optionally substituted with one or more groups R^{Cyc}, and wherein one or more —CH₂— units comprised in said alkylene are each optionally replaced by a group independently selected from —O—, —NH—, —N(C₁₋₅ alkyl)-, —CO—, —S—, —SO—, —SO₂—, and phen-1,2-diyl, wherein said phen-1,2-diyl is optionally substituted with one or more groups R^{Cyc};
each R^{A21} is independently selected from halogen, C₁₋₅ haloalkyl, —O(C₁₋₅ haloalkyl), —CN, —OR^{A22}, —NR^{A22}R^{A22}, —NR^{A22}OR^{A22}, —COR^{A22}, —COOR^{A22}, —OCOR^{A22}, —CONR^{A22}R^{A22}, —NR^{A22}COR^{A22}, —NR^{A22}COOR^{A22}, —OCONR^{A22}R^{A22}, —SR^{A22}, —SOR^{A22}, —SO₂R^{A22}, —SO₂NR^{A22}R^{A22}, —NR^{A22}SO₂R^{A22}, —SO₃R^{A22}, —NO₂, carbocyclyl, and heterocyclyl, wherein said carbocyclyl and said heterocyclyl are each optionally substituted with one or more groups R^{Cyc};
each R^{A22} is independently selected from hydrogen, C₁₋₅ alkyl, C₂₋₅ alkenyl, C₂₋₅ alkynyl, carbocyclyl, and heterocyclyl, wherein said alkyl, said alkenyl and said alkynyl are each optionally substituted with one or more groups R^{Alk}, and further wherein said carbocyclyl and said heterocyclyl are each optionally substituted with one or more groups R^{Cyc};
each R^{B1} is independently selected from C₁₋₅ alkyl, C₂₋₅ alkenyl, C₂₋₅ alkynyl, —(C₀₋₅ alkylene)-R^{B11}, —(C₂₋₅ alkenylene)-R^{B11}, —(C₂₋₅ alkynylene)-R^{B11}, and ═R^{B13}, wherein said alkyl, said alkenyl, said alkynyl, said alkylene, said alkenylene, and said alkynylene are each optionally substituted with one or more groups R^{Alk}, and further wherein one or more —CH₂— units comprised in said alkylene, said alkenylene, or said alkynylene are each optionally replaced by a group independently selected from —O—, —NH—, —N(C₁₋₅ alkyl)-, —CO—, —S—, —SO—, and —SO₂—;
each R^{B11} is independently selected from halogen, C₁₋₅ haloalkyl, —O(C₁₋₅ haloalkyl), —CN, —OR^{B12}, —NR^{B12}R^{B12}, —N⁺R^{B12}R^{B12}R^{B12}, —NR^{B12}OR^{B12}, —COR^{B12}, —COOR^{B12}, —OCOR^{B12}, —CONR^{B12}R^{B12}, —NR^{B12}COR^{B12}, —NR^{B12}COOR^{B12}, —OCONR^{B12}R^{B12}, —SR^{B12}, —SOR^{B12}, —SO₂R^{B12}, —SO₂NR^{B12}R^{B12}, —NR^{B12}SO₂R^{B12}, —SO₃R^{B12}, —NO₂, carbocyclyl, and heterocyclyl, wherein said carbocyclyl and said heterocyclyl are each optionally substituted with one or more groups R^{Cyc};
each R^{B12} is independently selected from hydrogen, C₁₋₅ alkyl, C₂₋₅ alkenyl, C₂₋₅ alkynyl, carbocyclyl, and heterocyclyl, wherein said alkyl, said alkenyl and said alkynyl are each optionally substituted with one or more groups R^{Alk}, and further wherein said carbocyclyl and said heterocyclyl are each optionally substituted with one or more groups R^{Cyc};
each R^{B13} is independently selected from ═O, ═S, and ═N—R^{B12};
each R^{Alk} is independently selected from —OH, —O(C₁₋₅ alkyl), —O(C₁₋₅ alkylene)-OH, —O(C₁₋₅ alkylene)-O(C₁₋₅ alkyl), —SH, —S(C₁₋₅ alkyl), —S(C₁₋₅ alkylene)-SH, —S(C₁₋₅ alkylene)-S(C₁₋₅ alkyl), —NH₂, —NH(C₁₋₅ alkyl), —N(C₁₋₅ alkyl)(C₁₋₅ alkyl), —NH—OH, —N(C₁₋₅ alkyl)-OH, —NH—O(C₁₋₅ alkyl), —N(C₁₋₅ alkyl)-O(C₁₋₅ alkyl), halogen, C₁₋₅ haloalkyl, —O(C₁₋₅ haloalkyl), —CN, —NO₂, —CHO, —CO(C₁₋₅ alkyl), —COOH, —COO(C₁₋₅ alkyl), —O—CO(C₁₋₅ alkyl), —CO—NH₂, —CO—NH(C₁₋₅ alkyl), —CO—N(C₁₋₅ alkyl)(C₁₋₅ alkyl), —NH—CO(C₁₋₅ alkyl), —N(C₁₋₅ alkyl)-CO(C₁₋₅ alkyl), —NH—COO(C₁₋₅ alkyl), —N(C₁₋₅ alkyl)-COO(C₁₋₅ alkyl), —O—CO—NH(C₁₋₅ alkyl), —O—CO—N(C₁₋₅ alkyl)(C₁₋₅ alkyl), —SO₂—NH₂, —SO₂—NH(C₁₋₅ alkyl), —SO₂—N(C₁₋₅ alkyl)(C₁₋₅ alkyl), —NH—SO₂—(C₁₋₅ alkyl), —N(C₁₋₅ alkyl)-SO₂—(C₁₋₅ alkyl), —SO₂—(C₁₋₅ alkyl), —SO—(C₁₋₅ alkyl), aryl, heteroaryl, cycloalkyl, heterocycloalkyl, and -L^{X}-R^{X}, wherein said aryl, said heteroaryl, said cycloalkyl, and said heterocycloalkyl are each optionally substituted with one or more groups independently selected from C₁₋₅ alkyl, C₂₋₅ alkenyl, C₂₋₅ alkynyl, halogen, C₁₋₅ haloalkyl, —O(C₁₋₅ haloalkyl), —CN, —OH, —O(C₁₋₅ alkyl), —SH, —S(C₁₋₅ alkyl), —NH₂, —NH(C₁₋₅ alkyl), and —N(C₁₋₅ alkyl)(C₁₋₅ alkyl);
each R^{Cyc} is independently selected from C₁₋₅ alkyl, C₂₋₅ alkenyl, C₂₋₅ alkynyl, —OH, —O(C₁₋₅ alkyl), —O(C₁₋₅ alkylene)-OH, —O(C₁₋₅ alkylene)-O(C₁₋₅ alkyl), —SH, —S(C₁₋₅ alkyl), —S(C₁₋₅ alkylene)-SH, —S(C₁₋₅ alkylene)-S(C₁₋₅ alkyl), —NH₂, —NH(C₁₋₅ alkyl), —N(C₁₋₅ alkyl)(C₁₋₅ alkyl), —NH—OH, —N(C₁₋₅ alkyl)-OH, —NH—O(C₁₋₅ alkyl), —N(C₁₋₅ alkyl)-O(C₁₋₅ alkyl), halogen, C₁₋₅ haloalkyl, —O(C₁₋₅ haloalkyl), —CN, —NO₂, —CHO, —CO(C₁₋₅ alkyl), —COOH, —COO(C₁₋₅ alkyl), —O—CO(C₁₋₅ alkyl), —CO—NH₂, —CO—NH(C₁₋₅ alkyl), —CO—N(C₁₋₅ alkyl)(C₁₋₅ alkyl), —NH—CO(C₁₋₅ alkyl), —N(C₁₋₅ alkyl)-CO(C₁₋₅ alkyl), —NH—COO(C₁₋₅ alkyl), —N(C₁₋₅ alkyl)-COO(C₁₋₅ alkyl), —O—CO—NH(C₁₋₅ alkyl), —O—CO—N(C₁₋₅ alkyl)(C₁₋₅ alkyl), —SO₂—NH₂, —SO₂—NH(C₁₋₅ alkyl), —SO₂—N(C₁₋₅ alkyl)(C₁₋₅ alkyl), —NH—SO₂—(C₁₋₅ alkyl), —N(C₁₋₅ alkyl)-SO₂—(C₁₋₅ alkyl), —SO₂—(C₁₋₅ alkyl), —SO—(C₁₋₅ alkyl), aryl, heteroaryl, cycloalkyl, heterocycloalkyl, and -L^{X}-R^{X}, wherein said aryl, said heteroaryl, said cycloalkyl, and said heterocycloalkyl are each optionally substituted with one or more groups independently selected from C₁₋₅ alkyl, C₂₋₅ alkenyl, C₂₋₅ alkynyl, halogen, C₁₋₅ haloalkyl, —O(C₁₋₅ haloalkyl), —CN, —OH, —O(C₁₋₅ alkyl), —SH, —S(C₁₋₅ alkyl), —NH₂, —NH(C₁₋₅ alkyl), and —N(C₁₋₅ alkyl)(C₁₋₅ alkyl);
each L^{X} is independently selected from a bond, C₁₋₅ alkylene, C₂₋₅ alkenylene, and C₂₋₅ alkynylene, wherein said alkylene, said alkenylene and said alkynylene are each optionally substituted with one or more groups independently selected from halogen, C₁₋₅ haloalkyl, —CN, —OH, —O(C₁₋₅ alkyl), —SH, —S(C₁₋₅ alkyl), —NH₂, —NH(C₁₋₅ alkyl), and —N(C₁₋₅ alkyl)(C₁₋₅ alkyl), and further wherein one or more —CH₂— units comprised in said alkylene, said alkenylene or said alkynylene are each optionally replaced by a group independently selected from —O—, —NH—, —N(C₁₋₅ alkyl)-, —CO—, —S—, —SO—, and —SO₂—; and
each R^{X} is independently selected from —OH, —O(C₁₋₅ alkyl), —O(C₁₋₅ alkylene)-OH, —O(C₁₋₅ alkylene)-O(C₁₋₅ alkyl), —SH, —S(C₁₋₅ alkyl), —S(C₁₋₅ alkylene)-SH, —S(C₁₋₅ alkylene)-S(C₁₋₅ alkyl), —NH₂, —NH(C₁₋₅ alkyl), —N(C₁₋₅ alkyl)(C₁₋₅ alkyl), —NH—OH, —N(C₁₋₅ alkyl)-OH, —NH—O(C₁₋₅ alkyl), —N(C₁₋₅ alkyl)-O(C₁₋₅ alkyl), halogen, C₁₋₅ haloalkyl, —O(C₁₋₅ haloalkyl), —CN, —NO₂, —CHO, —CO(C₁₋₅ alkyl), —COOH, —COO(C₁₋₅ alkyl), —O—CO(C₁₋₅ alkyl), —CO—NH₂, —CO—NH(C₁₋₅ alkyl), —CO—N(C₁₋₅ alkyl)(C₁₋₅ alkyl), —NH—CO(C₁₋₅ alkyl), —N(C₁₋₅ alkyl)-CO(C₁₋₅ alkyl), —NH—COO(C₁₋₅ alkyl), —N(C₁₋₅ alkyl)-COO(C₁₋₅ alkyl), —O—CO—NH(C₁₋₅ alkyl), —O—CO—N(C₁₋₅ alkyl)(C₁₋₅ alkyl), —SO₂—NH₂, —SO₂—NH(C₁₋₅ alkyl), —SO₂—N(C₁₋₅ alkyl)(C₁₋₅ alkyl), —NH—SO₂—(C₁₋₅ alkyl), —N(C₁₋₅ alkyl)-SO₂—(C₁₋₅ alkyl), —SO₂—(C₁₋₅ alkyl), —SO—(C₁₋₅ alkyl), aryl, heteroaryl, cycloalkyl, and heterocycloalkyl, wherein said aryl, said heteroaryl, said cycloalkyl, and said heterocycloalkyl are each optionally substituted with one or more groups independently selected from C₁₋₅ alkyl, C₂₋₅ alkenyl, C₂₋₅ alkynyl, halogen, C₁₋₅ haloalkyl, —O(C₁₋₅ haloalkyl), —CN, —OH, —O(C₁₋₅ alkyl), —SH, —S(C₁₋₅ alkyl), —NH₂, —NH(C₁₋₅ alkyl), and —N(C₁₋₅ alkyl)(C₁₋₅ alkyl);
or a pharmaceutically acceptable salt or solvate thereof;
wherein the following compounds are excluded from formula (I):
1-(((5-phenyl-4,5-dihydro-1H-imidazol-2-yl)thio)methyl)pyrrolidine;
1-(2-((5-phenyl-4,5-dihydro-1H-imidazol-2-yl)thio)ethyl)pyrrolidine;
1-(3-((5-phenyl-4,5-dihydro-1H-imidazol-2-yl)thio)propyl)pyrrolidine;
1-(((5-phenyl-4,5-dihydro-1H-imidazol-2-yl)thio)methyl)piperidine;
1-(2-((5-phenyl-4,5-dihydro-1H-imidazol-2-yl)thio)ethyl)piperidine;
1-(3-((5-phenyl-4,5-dihydro-1H-imidazol-2-yl)thio)propyl)piperidine;
1-(2-((4,5-dihydro-1H-imidazol-2-yl)thio)ethyl)pyrrolidine;
1-(3-((4,5-dihydro-1H-imidazol-2-yl)thio)propyl)pyrrolidine;
1-(4-((4,5-dihydro-1H-imidazol-2-yl)thio)butyl)pyrrolidine;
1-(2-((4,5-dihydro-1H-imidazol-2-yl)thio)ethyl)piperidine;
1-(3-((4,5-dihydro-1H-imidazol-2-yl)thio)propyl)piperidine;
1-(4-((4,5-dihydro-1H-imidazol-2-yl)thio)butyl)piperidine;
1-(2-((4,5-dihydro-1H-imidazol-2-yl)thio)ethyl)azepane;
1-(3-((4,5-dihydro-1H-imidazol-2-yl)thio)propyl)azepane;
1-(4-((4,5-dihydro-1H-imidazol-2-yl)thio)butyl)azepane;
1-(2-((1,4,5,6-tetrahydropyrimidin-2-yl)thio)ethyl)pyrrolidine;
1-(3-((1,4,5,6-tetrahydropyrimidin-2-yl)thio)propyl)pyrrolidine;
1-(4-((1,4,5,6-tetrahydropyrimidin-2-yl)thio)butyl)pyrrolidine;
1-(2-((1,4,5,6-tetrahydropyrimidin-2-yl)thio)ethyl)piperidine;
1-(3-((1,4,5,6-tetrahydropyrimidin-2-yl)thio)propyl)piperidine;
1-(4-((1,4,5,6-tetrahydropyrimidin-2-yl)thio)butyl)piperidine;
1-(2-((1,4,5,6-tetrahydropyrimidin-2-yl)thio)ethyl)azepane;
1-(3-((1,4,5,6-tetrahydropyrimidin-2-yl)thio)propyl)azepane;
1-(4-((1,4,5,6-tetrahydropyrimidin-2-yl)thio)butyl)azepane;
1-(2-((4,5,6,7-tetrahydro-1H-1,3-diazepin-2-yl)thio)ethyl)pyrrolidine;
1-(3-((4,5,6,7-tetrahydro-1H-1,3-diazepin-2-yl)thio)propyl)pyrrolidine;
1-(4-((4,5,6,7-tetrahydro-1H-1,3-diazepin-2-yl)thio)butyl)pyrrolidine;
1-(2-((4,5,6,7-tetrahydro-1H-1,3-diazepin-2-yl)thio)ethyl)piperidine;
1-(3-((4,5,6,7-tetrahydro-1H-1,3-diazepin-2-yl)thio)propyl)piperidine;
1-(4-((4,5,6,7-tetrahydro-1H-1,3-diazepin-2-yl)thio)butyl)piperidine;
1-(2-((4,5,6,7-tetrahydro-1H-1,3-diazepin-2-yl)thio)ethyl)azepane;
1-(3-((4,5,6,7-tetrahydro-1H-1,3-diazepin-2-yl)thio)propyl)azepane;
1-(4-((4,5,6,7-tetrahydro-1H-1,3-diazepin-2-yl)thio)butyl)azepane;
3-(1-(3-((4,5-dihydro-1H-imidazol-2-yl)thio)propyl)-1H-indol-3-yl)-4-(1-methyl-1H-indol-3-yl)-1H-pyrrole-2,5-dione;
2-(cyclopentylthio)-4,5-dihydro-1H-imidazole;
N-(piperidinomethyl)-2-[(piperidinomethyl)thio]-2-imidazoline;
N-((2-methylpiperidino)methyl)-2-[((2-methylpiperidino)methyl)thio]-2-imidazoline;
N-((3-methylpiperidino)methyl)-2-[((3-methylpiperidino)methyl)thio]-2-imidazoline;
N-((4-methylpiperidino)methyl)-2-[((4-methylpiperidino)methyl)thio]-2-imidazoline; and
N-((2-methyl-5-ethylpiperidino)methyl)-2-[((2-methyl-5-ethylpiperidino)methyl)thio]-2-imidazoline.

12. The compound of claim 1, wherein said compound is selected from:
3-(((6-chloro-1,4-dihydroquinazolin-2-yl)thio)methyl)-6,6-dimethyl-5,6-dihydroimidazo[2,1-b]thiazole;
7-chloro-3-(((5,5-dimethyl-4,5-dihydro-1H-imidazol-2-yl)thio)methyl)-5H-thiazolo[2,3-b]quinazoline;
3-(((3,4-dihydroquinazolin-2-yl)thio)methyl)-6,6-dimethyl-5,6-dihydroimidazo[2,1-b]thiazole;
7-chloro-3-(((4,5-dihydro-1H-imidazol-2-yl)thio)methyl)-5H-thiazolo[2,3-b]quinazoline;
3-(((5,5-dimethyl-4,5-dihydro-1H-imidazol-2-yl)thio)methyl)-5H-thiazolo[2,3-b]quinazoline;
3-(((7-chloro-1,4-dihydroquinazolin-2-yl)thio)methyl)-6,6-dimethyl-5,6-dihydroimidazo[2,1-b]thiazole;
3-(((2,5-dihydro-1H-benzo[e][1,3]diazepin-3-yl)thio)methyl)-6,6-dimethyl-5,6-dihydroimidazo[2,1-b]thiazole;
3-(((5,5-dimethyl-4,5-dihydro-1H-imidazol-2-yl)thio)methyl)-5,10-dihydrobenzo[e]thiazolo[3,2-a][1,3]diazepine;
8-chloro-3-(((5,5-dimethyl-4,5-dihydro-1H-imidazol-2-yl)thio)methyl)-5H-thiazolo[2,3-b]quinazoline;
3-(((6-chloro-1,4-dihydroquinazolin-2-yl)thio)methyl)-5,6-dihydroimidazo[2,1-b]thiazole;
3-(((3,4-dihydroquinazolin-2-yl)thio)methyl)-5,10-dihydrobenzo[e]thiazolo[3,2-a][1,3]diazepine;
3-(((1,4-dihydroquinazolin-2-yl)thio)methyl)-5H-thiazolo[2,3-b]quinazoline;
trans-3-(((3,4-dihydroquinazolin-2-yl)thio)methyl)-4a,5,6,7,8,8a-hexahydrobenzo[4,5]imidazo[2,1-b]thiazole;
6-(4-chlorophenyl)-3-(((3,4-dihydroquinazolin-2-yl)thio)methyl)-5,6-dihydroimidazo[2,1-b]thiazole;
6-cyclohexyl-3-(((3,4-dihydroquinazolin-2-yl)thio)methyl)-5,6-dihydroimidazo[2,1-b]thiazole;
trans-3-(((3,4-dihydroquinazolin-2-yl)thio)methyl)-5,6-diphenyl-5,6-dihydroimidazo[2,1-b]thiazole;
trans-3-(((3,4-dihydroquinazolin-2-yl)thio)methyl)-5,6-diphenyl-2,3,5,6-tetrahydroimidazo[2,1-b]thiazol-3-ol;
3-(((3,4-dihydroquinazolin-2-yl)thio)methyl)-6-fluoro-5H-thiazolo[2,3-b]quinazoline;
7-chloro-3-(((1,4-dihydroquinazolin-2-yl)thio)methyl)-5H-thiazolo[2,3-b]quinazoline;
3-(((5-benzyl-4,5-dihydro-1H-imidazol-2-yl)thio)methyl)-7-chloro-5H-thiazolo[2,3-b]quinazoline;
3-(((4,4-dimethyl-1,4-dihydroquinazolin-2-yl)thio)methyl)-5,10-dihydrobenzo[e]thiazolo[3,2-a][1,3]diazepine;
3-(((4-(4-chlorophenyl)-4,5-dihydro-1H-imidazol-2-yl)thio)methyl)-5,10-dihydrobenzo[e]thiazolo[3,2-a][1,3]diazepine;
3-(((5-fluoro-1,4-dihydroquinazolin-2-yl)thio)methyl)-5,10-dihydrobenzo[e]thiazolo[3,2-a][1,3]diazepine;
3-((((4S,5S)-4,5-diphenyl-4,5-dihydro-1H-imidazol-2-yl)thio)methyl)-5,10-dihydrobenzo[e]thiazolo[3,2-a][1,3]diazepine;
3-(((4,5-dihydro-1H-benzo[d][1,3]diazepin-2-yl)thio)methyl)-5,10-dihydrobenzo[e]thiazolo[3,2-a][1,3]diazepine;
3-(((4-cyclohexyl-4,5-dihydro-1H-imidazol-2-yl)thio)methyl)-5,10-dihydrobenzo[e]thiazolo[3,2-a][1,3]diazepine;
3-(((4-phenyl-3,4-dihydroquinazolin-2-yl)thio)methyl)-5,10-dihydrobenzo[e]thiazolo[3,2-a][1,3]diazepine;
3-(((3,4-dihydroquinazolin-2-yl)thio)methyl)-5H-pyrido[2,3-d]thiazolo[3,2-a]pyrimidine;
3-(((5-butyl-4,5-dihydro-1H-imidazol-2-yl)thio)methyl)-5H-thiazolo[2,3-b]quinazoline;
3-(((5-methyl-5-phenyl-4,5-dihydro-1H-imidazol-2-yl)thio)methyl)-5,10-dihydrobenzo[e]thiazolo[3,2-a][1,3]diazepine;
3-(((1,4-dihydropyrido[2,3-d]pyrimidin-2-yl)thio)methyl)-5,10-dihydrobenzo[e]thiazolo[3,2-a][1,3]diazepine;
3-((((3aR,7aR)-3a,4,5,6,7,7a-hexahydro-1H-benzo[d]imidazol-2-yl)thio)methyl)-5H-thiazolo[2,3-b]quinazoline;
3-(((5-benzyl-4,5-dihydro-1H-imidazol-2-yl)thio)methyl)-5H-thiazolo[2,3-b]quinazoline;
3-(((5-(4-methoxybenzyl)-5-methyl-4,5-dihydro-1H-imidazol-2-yl)thio)methyl)-5,10-dihydrobenzo[e]thiazolo[3,2-a][1,3]diazepine;
3-(((3,4-dihydroquinazolin-2-yl)thio)methyl)-5,6-dihydrobenzo[d]thiazolo[3,2-a][1,3]diazepine;
3-(((1-methyl-4,5-dihydro-1H-imidazol-2-yl)thio)methyl)-5H-thiazolo[2,3-b]quinazoline;
3-(((1-butyl-4,5-dihydro-1H-imidazol-2-yl)thio)methyl)-5,10-dihydrobenzo[e]thiazolo[3,2-a][1,3]diazepine;
3-(((3,4-dihydroquinazolin-2-yl)thio)methyl)-6-methyl-6-phenyl-5,6-dihydroimidazo[2,1-b]thiazole;
3-(((6-chloro-1,4-dihydroquinazolin-2-yl)thio)methyl)-5H-thiazolo[2,3-b]quinazoline;
7-chloro-3-((((3aR,7aR)-3a,4,5,6,7,7a-hexahydro-1H-benzo[d]imidazol-2-yl)thio)methyl)-5H-thiazolo[2,3-b]quinazoline;
3-(((5-butyl-4,5-dihydro-1H-imidazol-2-yl)thio)methyl)-7-chloro-5H-thiazolo[2,3-b]quinazoline;
8-chloro-3-(((1,4-dihydroquinazolin-2-yl)thio)methyl)-5H-thiazolo[2,3-b]quinazoline;
3-(((3,4-dihydroquinazolin-2-yl)thio)methyl)-5-phenyl-5H-thiazolo[2,3-b]quinazoline;
7-chloro-3-(((6-chloro-1,4-dihydroquinazolin-2-yl)thio)methyl)-5H-thiazolo[2,3-b]quinazoline;
7-chloro-3-(((1-methyl-4,5-dihydro-1H-imidazol-2-yl)thio)methyl)-5H-thiazolo[2,3-b]quinazoline;
3-(((3,4-dihydroquinazolin-2-yl)thio)methyl)-6-(4-methoxybenzyl)-6-methyl-5,6-dihydroimidazo[2,1-b]thiazole;
3-(((1-benzyl-4,5-dihydro-1H-imidazol-2-yl)thio)methyl)-7-chloro-5H-thiazolo[2,3-b]quinazoline;
7-chloro-3-(((1-isopropyl-4,5-dihydro-1H-imidazol-2-yl)thio)methyl)-5H-thiazolo[2,3-b]quinazoline;
7-chloro-3-(((1,5,6,7,8,8a-hexahydroimidazo[1,5-a]pyridin-3-yl)thio)methyl)-5H-thiazolo[2,3-b]quinazoline;
1-(2-((5-benzyl-4,5-dihydro-1H-imidazol-2-yl)thio)ethyl)piperidine;
2-(((5-benzyl-4,5-dihydro-1H-imidazol-2-yl)thio)methyl)imidazo[1,2-a]pyrimidine;
5-benzyl-2-((3-(pyrrolidin-1-yl)propyl)thio)-4,5-dihydro-1H-imidazole;
5-benzyl-2-(((1-methylpyrrolidin-2-yl)methyl)thio)-4,5-dihydro-1H-imidazole;
5-benzyl-2-((2-(pyrrolidin-1-yl)ethyl)thio)-4,5-dihydro-1H-imidazole;
4-(3-((5-benzyl-4,5-dihydro-1H-imidazol-2-yl)thio)propyl)pyridine;
4-(((5-benzyl-4,5-dihydro-1H-imidazol-2-yl)thio)methyl)pyridine;
5-benzyl-2-((2-(1-methylpyrrolidin-2-yl)ethyl)thio)-4,5-dihydro-1H-imidazole;
1-(2-((5-benzyl-4,5-dihydro-1H-imidazol-2-yl)thio)ethyl)azepane;
6-chloro-2-((2-(pyrrolidin-1-yl)ethyl)thio)-1,4-dihydroquinazoline;
6-chloro-2-((4-(pyrrolidin-1-yl)butyl)thio)-1,4-dihydroquinazoline;
2-(((5-benzyl-4,5-dihydro-1H-imidazol-2-yl)thio)methyl)-4-chlorothieno[3,2-c]pyridine;
3-(((3,4-dihydroquinazolin-2-yl)thio)methyl)-6,7-dimethoxy-2,3-dihydrobenzo[4,5]imidazo[2,1-b]thiazol-3-ol;
3-(((3,4-dihydroquinazolin-2-yl)thio)methyl)-6-(thiophen-2-ylmethyl)-5,6-dihydroimidazo[2,1-b]thiazole;
7-chloro-3-(((5-(thiophen-2-ylmethyl)-4,5-dihydro-1H-imidazol-2-yl)thio)methyl)-5H-thiazolo[2,3-b]quinazoline;
6-benzyl-3-(((3,4-dihydroquinazolin-2-yl)thio)methyl)-5,6-dihydroimidazo[2,1-b]thiazole;
3-(((7-bromo-1,4-dihydroquinazolin-2-yl)thio)methyl)-7-chloro-5H-thiazolo[2,3-b]quinazoline;
3-(((6-bromo-1,4-dihydroquinazolin-2-yl)thio)methyl)-7-chloro-5H-thiazolo[2,3-b]quinazoline;
3-(((4,6-diazaspiro[2.4]hept-5-en-5-yl)thio)methyl)-7-chloro-5H-thiazolo[2,3-b]quinazoline;
7-bromo-3-(((1,4-dihydroquinazolin-2-yl)thio)methyl)-5H-thiazolo[2,3-b]quinazoline;
8-bromo-3-(((1,4-dihydroquinazolin-2-yl)thio)methyl)-5H-thiazolo[2,3-b]quinazoline;
2-((2-(isoindolin-2-yl)ethyl)thio)-3,4-dihydroquinazoline;
7-chloro-3-(((5-methyl-5-phenyl-4,5-dihydro-1H-imidazol-2-yl)thio)methyl)-5H-thiazolo[2,3-b]quinazoline;
3-(((4,4-dimethyl-1,4-dihydroquinazolin-2-yl)thio)methyl)-6-fluoro-5H-thiazolo[2,3-b]quinazoline;
2-((2-(5-chloro-1H-indol-1-yl)ethyl)thio)-3,4-dihydroquinazoline;
7-chloro-3-(((4,4-dimethyl-1,4-dihydroquinazolin-2-yl)thio)methyl)-5H-thiazolo[2,3-b]quinazoline;
7-chloro-3-(((4,5-dihydro-1H-benzo[d][1,3]diazepin-2-yl)thio)methyl)-5H-thiazolo[2,3-b]quinazoline;
2-((2-(pyrrolidin-1-yl)ethyl)thio)-1,4-dihydroquinazoline;
4,4-dimethyl-2-((2-(pyrrolidin-1-yl)ethyl)thio)-1,4-dihydroquinazoline;
2-bromo-7-chloro-3-(((1,4-dihydroquinazolin-2-yl)thio)methyl)-5H-thiazolo[2,3-b]quinazoline;
7-chloro-3-(((5-fluoro-1,4-dihydroquinazolin-2-yl)thio)methyl)-5H-thiazolo[2,3-b]quinazoline;
6-chloro-3-(((1,4-dihydroquinazolin-2-yl)thio)methyl)-5H-thiazolo[2,3-b]quinazoline;
3-(((1,4-dihydroquinazolin-2-yl)thio)methyl)-8-fluoro-5H-thiazolo[2,3-b]quinazoline;
7-chloro-3-(((6-fluoro-1,4-dihydroquinazolin-2-yl)thio)methyl)-5H-thiazolo[2,3-b]quinazoline;
3-(((1,4-dihydroquinazolin-2-yl)thio)methyl)-7-fluoro-5H-thiazolo[2,3-b]quinazoline;
9-bromo-3-(((1,4-dihydroquinazolin-2-yl)thio)methyl)-5H-thiazolo[2,3-b]quinazoline;
7-chloro-3-(((1,4-dihydroquinazolin-2-yl)thio)methyl)-9-fluoro-5H-thiazolo[2,3-b]quinazoline;
6-benzyl-3-(((4,4-dimethyl-1,4-dihydroquinazolin-2-yl)thio)methyl)-5,6-dihydroimidazo[2,1-b]thiazole;
6-benzyl-3-(((4,5-dihydro-1H-benzo[d][1,3]diazepin-2-yl)thio)methyl)-5,6-dihydroimidazo[2,1-b]thiazole;
6-benzyl-3-(((7-fluoro-1,4-dihydroquinazolin-2-yl)thio)methyl)-5,6-dihydroimidazo[2,1-b]thiazole;
2-((2-(azepan-1-yl)ethyl)thio)-1,4-dihydroquinazoline;
2-((2-(piperidin-1-yl)ethyl)thio)-1,4-dihydroquinazoline;
3-(((8-bromo-1,4-dihydroquinazolin-2-yl)thio)methyl)-7-chloro-5H-thiazolo[2,3-b]quinazoline;
6-benzyl-3-(((3-butyl-3,4-dihydroquinazolin-2-yl)thio)methyl)-5,6-dihydroimidazo[2,1-b]thiazole;
6-(4-chlorobenzyl)-3-(((1,4-dihydroquinazolin-2-yl)thio)methyl)-5,6-dihydroimidazo[2,1-b]thiazole;
3-(((1,4-dihydroquinazolin-2-yl)thio)methyl)-5,5-dimethyl-5H-thiazolo[2,3-b]quinazoline;
3-(((3,4-dihydroquinazolin-2-yl)thio)methyl)benzo[4,5]imidazo[2,1-b]thiazole;
3-(((3,4-dihydroquinazolin-2-yl)thio)methyl)-6,7-dimethoxybenzo[4,5]imidazo[2,1-b]thiazole;
4,4-dimethyl-2-((1-methylpyrrolidin-3-yl)thio)-1,4-dihydroquinazoline;
6-benzyl-3-(((1-butyl-1,4-dihydroquinazolin-2-yl)thio)methyl)-5,6-dihydroimidazo[2,1-b]thiazole;
2-((1-methylpyrrolidin-3-yl)thio)-1,4-dihydroquinazoline;
2-((1-phenylpyrrolidin-3-yl)thio)-1,4-dihydroquinazoline;
2-((1-(2,2-difluoroethyl)pyrrolidin-3-yl)thio)-1,4-dihydroquinazoline;
6-chloro-2-((1-methylpyrrolidin-3-yl)thio)-1,4-dihydroquinazoline;
2-((1-ethylpyrrolidin-3-yl)thio)-1,4-dihydroquinazoline;
2-((1-methylpyrrolidin-3-yl)thio)-4,5-dihydro-1H-benzo[d][1,3]diazepine;
3-((1-phenylpyrrolidin-3-yl)thio)-2,5-dihydro-1H-benzo[e][1,3]diazepine;
2-((1-phenylpyrrolidin-3-yl)thio)-4,5-dihydro-1H-benzo[d][1,3]diazepine;
2-(((1-methylpyrrolidin-2-yl)methyl)thio)-1,4-dihydroquinazoline;
(S)-6-((1H-indol-3-yl)methyl)-3-(((1,4-dihydroquinazolin-2-yl)thio)methyl)-5,6-dihydroimidazo[2,1-b]thiazole;
6-benzyl-3-(((1,4-dihydroquinazolin-2-yl)thio)methyl)-2-iodo-5,6-dihydroimidazo[2,1-b]thiazole;
(S)-6-(3-chlorobenzyl)-3-(((1,4-dihydroquinazolin-2-yl)thio)methyl)-5,6-dihydroimidazo[2,1-b]thiazole;
3-(((1,4-dihydroquinazolin-2-yl)thio)methyl)-6-(3-methylbenzyl)-5,6-dihydroimidazo[2,1-b]thiazole;
6-benzyl-3-(((4-methyl-1,4-dihydroquinazolin-2-yl)thio)methyl)-5,6-dihydroimidazo[2,1-b]thiazole;
6-benzyl-3-(((6-chloro-1,4-dihydroquinazolin-2-yl)thio)methyl)-5,6-dihydroimidazo[2,1-b]thiazole;
2-((2-(indolin-1-yl)ethyl)thio)-1,4-dihydroquinazoline;
4-chloro-2-(((1,4-dihydroquinazolin-2-yl)thio)methyl)thieno[3,2-c]pyridine;
6-benzyl-3-(((5-fluoro-1,4-dihydroquinazolin-2-yl)thio)methyl)-5,6-dihydroimidazo[2,1-b]thiazole;
6-benzyl-3-(((5-chloro-1,4-dihydroquinazolin-2-yl)thio)methyl)-5,6-dihydroimidazo[2,1-b]thiazole;
6-benzyl-3-(((7-bromo-1,4-dihydroquinazolin-2-yl)thio)methyl)-5,6-dihydroimidazo[2,1-b]thiazole;
3-(((1,4-dihydroquinazolin-2-yl)thio)methyl)-6-phenyl-5,6-dihydroimidazo[2,1-b]thiazole;
3-(((1,4-dihydroquinazolin-2-yl)thio)methyl)-6-(3-fluorobenzyl)-5,6-dihydroimidazo[2,1-b]thiazole;
3-(((1,4-dihydroquinazolin-2-yl)thio)methyl)-6-(4-methylbenzyl)-5,6-dihydroimidazo[2,1-b]thiazole;
6-(2-chlorobenzyl)-3-(((1,4-dihydroquinazolin-2-yl)thio)methyl)-5,6-dihydroimidazo[2,1-b]thiazole;
(R)-3-(((1,4-dihydroquinazolin-2-yl)thio)methyl)-6-(4-methoxybenzyl)-5,6-dihydroimidazo[2,1-b]thiazole;
2-((2-(3,3-difluoropyrrolidin-1-yl)ethyl)thio)-1,4-dihydroquinazoline;
3-(((1,4-dihydroquinazolin-2-yl)thio)methyl)-6-phenethyl-5,6-dihydroimidazo[2,1-b]thiazole;
2-((2-(3-methoxypyrrolidin-1-yl)ethyl)thio)-1,4-dihydroquinazoline;
2-((2-(2-phenylpyrrolidin-1-yl)ethyl)thio)-1,4-dihydroquinazoline;
2-((2-(pyrrolidin-1-yl)propyl)thio)-1,4-dihydroquinazoline;
2-((2-(2-methylpyrrolidin-1-yl)ethyl)thio)-1,4-dihydroquinazoline;
5-methyl-5-phenyl-2-((2-(pyrrolidin-1-yl)ethyl)thio)-4,5-dihydro-1H-imidazole;
2-((2-(1,1-difluoro-5-azaspiro[2.4]heptan-5-yl)ethyl)thio)-3,4-dihydroquinazoline;
2-((2-((1R,5S)-8-azabicyclo[3.2.1]octan-8-yl)ethyl)thio)-3,4-dihydroquinazoline;
6,7,8-triiodo-2-((2-(pyrrolidin-1-yl)ethyl)thio)-1,4-dihydroquinazoline;
1-(2-((1,4-dihydroquinazolin-2-yl)thio)ethyl)pyrrolidin-2-one;
2-((3-(pyrrolidin-1-yl)propyl)thio)-1,4-dihydroquinazoline;
2-((4-(pyrrolidin-1-yl)butyl)thio)-1,4-dihydroquinazoline;
2-((2-(3-methylpyrrolidin-1-yl)ethyl)thio)-1,4-dihydroquinazoline;
(1S,4S)-5-(2-((1,4-dihydroquinazolin-2-yl)thio)ethyl)-2-oxa-5-azabicyclo[2.2.1]heptane;
2-((2-(3-phenylpyrrolidin-1-yl)ethyl)thio)-1,4-dihydroquinazoline;
2-(((2R)-2-(pyrrolidin-1-yl)cyclopentyl)thio)-1,4-dihydroquinazoline;
2-((2-(2-azaspiro[4.4]nonan-2-yl)ethyl)thio)-1,4-dihydroquinazoline;
2-((2-(3-(benzyloxy)pyrrolidin-1-yl)ethyl)thio)-1,4-dihydroquinazoline;
1-(2-((1,4-dihydroquinazolin-2-yl)thio)ethyl)pyrrolidine-3-carboxylic acid;
2-((2-(1-methylpyrrolidin-3-yl)ethyl)thio)-1,4-dihydroquinazoline;
(1R,4R)-5-(2-((1,4-dihydroquinazolin-2-yl)thio)ethyl)-2-oxa-5-azabicyclo[2.2.1]heptane;
4-((1,4-dihydroquinazolin-2-yl)thio)-1-(pyrrolidin-1-yl)butan-1-one;
2-(((2R)-2-(pyrrolidin-1-yl)cyclohexyl)thio)-1,4-dihydroquinazoline;
5-fluoro-2-((4-(pyrrolidin-1-yl)butyl)thio)-1,4-dihydroquinazoline;
7-chloro-2-((4-(pyrrolidin-1-yl)butyl)thio)-1,4-dihydroquinazoline;
7-fluoro-2-((4-(pyrrolidin-1-yl)butyl)thio)-1,4-dihydroquinazoline;
6-fluoro-2-((4-(pyrrolidin-1-yl)butyl)thio)-1,4-dihydroquinazoline;
8-chloro-2-((4-(pyrrolidin-1-yl)butyl)thio)-1,4-dihydroquinazoline;
2-((2-(3-benzylpyrrolidin-1-yl)ethyl)thio)-1,4-dihydroquinazoline;
4-(2-((1,4-dihydroquinazolin-2-yl)thio)ethyl)morpholine;
(S)-2-((2-(3-fluoropyrrolidin-1-yl)ethyl)thio)-1,4-dihydroquinazoline;
(R)-2-((2-(3-fluoropyrrolidin-1-yl)ethyl)thio)-1,4-dihydroquinazoline;
6-chloro-2-((2-(1-methylpyrrolidin-2-yl)ethyl)thio)-1,4-dihydroquinazoline;
2-((4-(pyrrolidin-1-yl)butyl)thio)-4,5-dihydro-1H-benzo[d][1,3]diazepine;
4,4-dimethyl-2-((4-(pyrrolidin-1-yl)butyl)thio)-1,4-dihydroquinazoline;
6-chloro-2-((3-(pyrrolidin-1-yl)propyl)thio)-1,4-dihydroquinazoline;
6-chloro-2-((4-(pyrrolidin-1-yl)pentyl)thio)-1,4-dihydroquinazoline;
6-bromo-2-((4-(pyrrolidin-1-yl)butyl)thio)-1,4-dihydroquinazoline;
6-chloro-2-((4-(piperidin-1-yl)butyl)thio)-1,4-dihydroquinazoline;
2-((4-(pyrrolidin-1-yl)pentyl)thio)-1,4-dihydroquinazoline;
(S)-6-chloro-2-((2-(pyrrolidin-1-yl)propyl)thio)-1,4-dihydroquinazoline;
(R)-6-chloro-2-((2-(pyrrolidin-1-yl)propyl)thio)-1,4-dihydroquinazoline;
(S)-6-chloro-2-((1-(pyrrolidin-1-yl)propan-2-yl)thio)-1,4-dihydroquinazoline;
5-(4-methoxybenzyl)-5-methyl-2-((4-(pyrrolidin-1-yl)butyl)thio)-4,5-dihydro-1H-imidazole;
5-methyl-5-phenyl-2-((4-(pyrrolidin-1-yl)butyl)thio)-4,5-dihydro-1H-imidazole;
3-((4-(pyrrolidin-1-yl)butyl)thio)-2,5-dihydro-1H-benzo[e][1,3]diazepine;
4,4-dimethyl-2-((4-(pyrrolidin-1-yl)butyl)thio)-4,5-dihydro-1H-imidazole;
2-((4-(pyrrolidin-1-yl)butyl)thio)-1,4,5,6-tetrahydropyrimidine;
6-chloro-2-((3-(1-methylpyrrolidin-2-yl)propyl)thio)-1,4-dihydroquinazoline;
2-((4-(pyrrolidin-1-yl)butyl)thio)-4,5-dihydro-1H-imidazole;
2-((4-(1H-imidazol-1-yl)butyl)thio)-6-chloro-1,4-dihydroquinazoline;
6-chloro-2-((2-(1-methylpyrrolidin-3-yl)ethyl)thio)-1,4-dihydroquinazoline;
2-((4-(pyrrolidin-1-yl)butyl)thio)-4,5,6,7-tetrahydro-1H-1,3-diazepine;
5,5-dimethyl-2-((4-(pyrrolidin-1-yl)butyl)thio)-1,4,5,6-tetrahydropyrimidine;
2′-((4-(pyrrolidin-1-yl)butyl)thio)-1′H-spiro[cyclopropane-1,4′-quinazoline];
5-benzyl-2-((4-(pyrrolidin-1-yl)butyl)thio)-4,5-dihydro-1H-imidazole;
2-((2-(pyrrolidin-1-yl)ethyl)thio)-4,5-dihydro-1H-benzo[d][1,3]diazepine;
5-(4-methoxybenzyl)-5-methyl-2-((2-(pyrrolidin-1-yl)ethyl)thio)-4,5-dihydro-1H-imidazole;
2-((2-(pyrrolidin-1-yl)ethyl)thio)-1,4,4a,5,6,7,8,8a-octahydroquinazoline;
5-((4-(pyrrolidin-1-yl)butyl)thio)-4,6-diazaspiro[2.4]hept-5-ene;
3-((2-(pyrrolidin-1-yl)ethyl)thio)-2,5-dihydro-1H-benzo[e][1,3]diazepine;
5-((2-(pyrrolidin-1-yl)ethyl)thio)-4,6-diazaspiro[2.4]hept-5-ene;
2-((pyridin-4-ylmethyl)thio)-4,5-dihydro-1H-benzo[d][1,3]diazepine;
3-((pyridin-4-ylmethyl)thio)-2,5-dihydro-1H-benzo[e][1,3]diazepine;
2-((3-(pyrrolidin-1-yl)propyl)thio)-4,5-dihydro-3H-benzo[d][1,3]diazepine;
2-((2-(3,4-dihydroquinolin-1(2H)-yl)ethyl)thio)-4,5-dihydro-1H-benzo[d][1,3]diazepine;
2-((2-(indolin-1-yl)ethyl)thio)-4,5-dihydro-1H-benzo[d][1,3]diazepine;
3-((pyridin-3-ylmethyl)thio)-2,5-dihydro-1H-benzo[e][1,3]diazepine;
3-((3-(pyrrolidin-1-yl)propyl)thio)-2,5-dihydro-1H-benzo[e][1,3]diazepine;
3-((2-(indolin-1-yl)ethyl)thio)-2,5-dihydro-1H-benzo[e][1,3]diazepine;
3-(((1,4-dihydroquinazolin-2-yl)thio)methyl)-6-(4-fluorobenzyl)-5,6-dihydroimidazo[2,1-b]thiazole;
2-((2-cyclopentylethyl)thio)-1,4-dihydroquinazoline;
tert-butyl (S)-3-((4,5-dihydro-1H-benzo[d][1,3]diazepin-2-yl)thio)pyrrolidine-1-carboxylate;
(S)-2-(pyrrolidin-3-ylthio)-4,5-dihydro-3H-benzo[d][1,3]diazepine;
(S)-2-((1-methylpyrrolidin-3-yl)thio)-4,5-dihydro-3H-benzo[d][1,3]diazepine;
or a pharmaceutically acceptable salt or solvate of any one of these compounds.

13. A pharmaceutical composition comprising the compound of any one of claims 1 to 12 and a pharmaceutically acceptable excipient.

14. The compound of any one of claims 1 to 12 or the pharmaceutical composition of claim 13 for use in the treatment or prevention of an inflammatory disorder, an autoimmune disorder, an autoinflammatory disorder, or an interferonopathy.

15. The compound for use according to claim 14 or the pharmaceutical composition for use according to claim 14, wherein the inflammatory disorder, autoimmune disorder, autoinflammatory disorder or interferonopathy to be treated or prevented is selected from Aicardi-Goutières syndrome, familial chilblain lupus, Singleton-Merten syndrome, proteasome-associated autoinflammatory syndrome, deficiency of adenosine deaminase 2, retinal vasculopathy with cerebral leukodystrophy, STING-associated vasculopathy with onset in infancy, spondyloenchondrodysplasia, ISG15 deficiency, an interferonopathy associated with genetic dysfunction, familial Mediterranean fever, TNF receptor associated periodic fever syndrome, periodic fever, aphthous stomatitis, pharyngitis, cervical adenitis, pyogenic arthritis, pyoderma gangrenosum, acne, Blau syndrome, neonatal onset multisystem inflammatory disease, familial cold autoinflammatory syndrome, hyperimmunoglobulinemia D with periodic fever syndrome, Muckle-Wells syndrome, chronic infantile neurological cutaneous and articular syndrome, deficiency of interleukin-1 receptor antagonist, haploinsufficiency of A20, deficiency of IL-36 receptor antagonist, CARD14-mediated psoriasis, inflammatory bowel disease, PLCG2-associated autoinflammation, antibody deficiency and immune dysregulation, an inflammatory disorder associated with genetic dysfunction, rheumatoid arthritis, spondyloarthritis, osteoarthritis, gout, idiopathic juvenile arthritis, psoriatic arthritis, eczema, psoriasis, scleroderma, systemic lupus erythematosus, Sjögren's syndrome, dermatomyositis, overlapping myositis, mixed connective tissue disease, undifferentiated connective tissue disease, chronic obstructive pulmonary disease, bowel inflammation, Crohn disease, Behçet's disease, ulcerative colitis, sepsis, macrophages activation syndrome, acute respiratory distress syndrome, type II diabetes, asthma, chronic wounds, autism, multiple sclerosis, Alzheimer's disease, Parkinson's disease, chronic inflammatory demyelinating polyneuropathy, juvenile dermatomyositis, and an inflammatory complication associated with a viral infection.

16. The compound of any one of claims 1 to 12 or the pharmaceutical composition of claim 13 for use in the treatment or prevention of rheumatoid arthritis, dermatomyositis or systemic lupus erythematosus.

17. In vitro use of a compound as defined in any one of claims 1 to 12 as a CXCR4 modulator.
